(19) United States
(12) Patent Application Publication
(10) Pub. No.: US 2020/0085869 A1
(43) Pub. Date: Mar. 19, 2020

(54) THERAPEUTIC REGIMENS FOR CHIMERIC ANTIGEN RECEPTOR THERAPIES

(71) Applicants: Novartis AG, Basel (CH); The Trustees of the University of Pennsylvania, Philadelphia, PA (US)

(72) Inventors: Stephen Schuster, Springfield, PA (US); Lamis Eldjerou, Morris Plains, NJ (US); John Peter Plastaras, Philadelphia, PA (US); William Tristram Arscott, Portland, OR (US); Stephan Grupp, Havertown, PA (US)

(21) Appl. No.: 16/413,158

(22) Filed: May 15, 2019

Related U.S. Application Data
(60) Provisional application No. 62/748,019, filed on Oct. 19, 2018.
(60) Provisional application No. 62/672,329, filed on May 16, 2018.

(51) Int. Cl. A61K 35/17; C07K 14/705; A61P 35/04; C07K 14/725; C12N 15/86

(52) U.S. Cl. CPC A61K 35/17; C07K 14/70507; A61P 35/04; A61K 45/06; C07K 14/70521; C07K 14/7051; C12N 15/86; C07K 14/70596

(57) ABSTRACT

The invention provides a method of treating an adult subject having a hematological cancer, comprising administering to the subject selected dosage regimens comprising a plurality of immune effector cells expressing a CAR molecule.

### CROSS-REFERENCE TO RELATED APPLICATIONS

This application claims the benefit of U.S. Provisional Application 62/672,329 filed on May 16, 2018, and U.S. Provisional Application 62/748,019 filed on Oct. 19, 2018, the entire contents of each of which are hereby incorporated by reference.

### FIELD OF THE INVENTION

The present invention relates, at least in part, to dosage regimens for immune cells engineered to express a Chimeric Antigen Receptor (CAR).

### BACKGROUND OF THE INVENTION

Many patients with B cell malignancies are incurable with standard therapy. In addition, traditional treatment options often have serious side effects. Attempts have been made in cancer immunotherapy, however, several obstacles render this a very difficult goal to achieve clinical effectiveness. Although hundreds of so-called tumor antigens have been identified, these are generally derived from self and thus are poorly immunogenic. Furthermore, tumors use several mechanisms to render themselves hostile to the initiation and propagation of immune attack.

Recent developments using chimeric antigen receptor (CAR) modified autologous T cell (CART) therapy, which relies on redirecting T cells to a suitable cell-surface molecule on cancer cells such as B cell malignancies, show promising results in harnessing the power of the immune system to treat B cell malignancies and other cancers (see, e.g., Sadelain et al., Cancer Discovery 3:388-398 (2013)). The clinical results of the murine derived CART19 (i.e., “CTL019”) have shown promise in establishing complete remissions in patients suffering with CLL as well as in childhood ALL (see, e.g., Kalos et al., Sci Transl Med 3:95ra73 (2011), Porter et al., NEJM 365:725-733 (2011), Grupp et al., NEJM 368:1509-1518 (2013)). Besides the ability for the chimeric antigen receptor on the genetically modified T cells to recognize and destroy the targeted cells, a successful therapeutic T cell therapy needs to have the ability to proliferate and persist over time, in order to survey for leukemic relapse. The variable quality of T cells, resulting from anergy, suppression, or exhaustion, will have effects on CAR-transformed T cells' performance, over which skilled practitioners have limited control at this time. To be effective, CAR transformed patient T cells need to persist and maintain the ability to proliferate in response to the cognate antigen. It has been shown that ALL patient T cells perform can do this with CART19 comprising a murine scFv (see, e.g., Grupp et al., NEJM 368:1509-1518 (2013)).

### SUMMARY OF THE INVENTION

The disclosure features, at least in part, CAR dosing regimens comprising combinations of CAR therapy with, e.g., radiotherapy (radiation therapy) or stem cell therapy (SCT). In one embodiment, the invention pertains to a method of treating a subject having a cancer (e.g., a hematological cancer, e.g., B-Acute Lymphocytic Leukemia (B cell ALL) or Non-Hogkin Lymphom (NHL)), comprising administering to the subject a CAR-expressing cell therapy, e.g., a CAR19 expressing cell therapy, in combination with radiotherapy or SCT. Additionally, disclosed herein is a method of treating, e.g., preventing cytokine release syndrome (CRS) in a subject with a CAR-expressing cell therapy by administering to the subject radiotherapy before the administration of the CAR-expressing cell therapy. The methods disclosed herein can result, e.g., in improved efficacy of the CAR-expressing cell therapy and reduced side effects, e.g., CRS, associated with the CAR-expressing cell therapy. Accordingly, methods comprising a plurality of CAR-expressing cells, as well as methods of monitoring, or making, a CAR-expressing therapy are disclosed.

Accordingly, in one aspect, disclosed herein is a method of treating a subject comprising administering to the subject a CAR-expressing cell therapy, e.g., a CAR19 expressing cell therapy, wherein the CAR-expressing cell therapy is administered less than 30 days, e.g., less than 29, 28, 27, 26, 25, 24, 23, 22, 21, 20, 19, 18, 17, 16, 15, 14, 13, 12, 11, 10, 9, 8, 7, 6, 5, 4, 3, 2, or 1 days, after administration of a lymphodepleting therapy comprising radiotherapy.

In another aspect, the disclosure provides a method of treating, e.g., preventing, cytokine release syndrome (CRS) with a CAR-expressing cell therapy, e.g., a CAR19 expressing cell therapy, in a subject in need thereof, comprising administering to the subject a lymphodepleting therapy comprising radiotherapy, thereby preventing CRS in the subject.

In some embodiments, the radiotherapy is administered less than 30 days, e.g., less than 29, 28, 27, 26, 25, 24, 23, 22, 21, 20, 19, 18, 17, 16, 15, 14, 13, 12, 11, 10, 9, 8, 7, 6, 5, 4, 3, 2, or 1 days, prior to the administration of the CAR-expressing cell therapy.

In some embodiments, the subject (i) is at risk of developing, has, or is diagnosed with CRS; (ii) is identified or has previously been identified as being at risk for CRS; and/or (iii) has been, is being, or will be administered a CAR therapy, e.g., a CD19 CAR-expressing cell. In some embodiments, the subject is selected based on
(i) risk of developing CRS, and/or
(ii) whether the subject has been, is being, or will be administered a CAR therapy (e.g., CD19 CAR-expressing cell).

In some embodiments, the subject is selected for administration of radiotherapy if the subject is at risk of developing CRS. In some embodiments, the CRS is a severe CRS, e.g., grade 4 or 5 CRS. In some embodiments, the CRS is a less than severe CRS, e.g., grade 1, 2, or 3 CRS.

In some embodiments, the subject is selected for administration of radiotherapy if the subject will be administered a CAR therapy, e.g., a CD19 CAR-expressing cell.

In some embodiments of any of the methods disclosed herein, the lymphodepleting therapy comprises radiotherapy. In some embodiments, the radiotherapy is administered at a dose of about 1-100 Gy, e.g., about 1-10, 10-20, 20-30, 30-40, 40-50, 50-60, 60-70, 80-90, or 90-100 Gy, or about 1-99, 5-90, 10-85, 15-80, 20-75, 25-70, 30-65, 35-60, 40-55 or 45-50 Gy. In some embodiments, the radiotherapy is administered at a dose of about 50 Gy, e.g, about 49, 48, 47, 46, 45, 44, 43, 42, 41, 40, 35, 30, 25, 24, 23, 22, 21, 20, 15, 10, 9, 8, 7, 6, 5, 4, 3, 2.9, 2.8, 2.7, 2.6, 2.5, 2.4, 2.3, 2.2, 2.1, 2 or 1 Gy. In some embodiments, the radiotherapy is administered at a dose of about 40 Gy, e.g., 40 Gy. In some embodiments, the radiotherapy is administered at a dose of about 22 Gy, e.g., 22 Gy. In some embodiments, the radiotherapy is administered at a dose of about 4 Gy, e.g., 4 Gy. In some embodiments, the radiotherapy is administered at a dose of about 2.2 Gy, e.g., 2.2 Gy.

In some embodiments of any of the methods disclosed herein, the radiotherapy is administered as a single dose, e.g., at a dose described herein.

In some embodiments of any of the methods disclosed herein, the radiotherapy is administered as a fractionated dose, e.g., one or more doses (e.g., two, three or four partial doses). In some embodiments, the radiotherapy consists of a total dose administered as a fractionated dose, e.g., one or more doses (e.g., two, three or four partial doses). In some embodiments, the radiotherapy is administered as two doses, e.g., two fractionated doses, e.g., a first dose and a second dose. In some embodiments, the first dose, e.g., first fractionated dose, is at least about 40 fold, e.g., about 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, or 1.5 fold, higher than the subsequent dose, e.g., second dose, e.g., second fractionated dose. In some embodiments, the first dose, e.g., first fractionated dose, is at least about 20 fold higher than the subsequent dose, e.g., second dose, e.g., second fractionated dose. In some embodiments, the first dose, e.g., first fractionated dose, is at least about 10 fold higher than the subsequent dose, e.g., second dose, e.g., second fractionated dose. In some embodiments, the first dose, e.g., first fractionated dose, is at least about 2 fold higher than the subsequent dose, e.g., second dose, e.g., second fractionated dose. In some embodiments, the first dose, e.g., first fractionated dose is about 40 Gy, and the second dose, e.g., second fractionated dose, is about 2 Gy. In some embodiments, the first dose, e.g., first fractionated dose is about 4 Gy, and the second dose, e.g., second fractionated dose, is about 2 Gy. In some embodiments, the first dose, e.g., first fractionated dose is about 22 Gy, and the second dose, e.g., second fractionated dose, is about 2.2 Gy.

In some embodiments, the one or more fractionated doses of radiotherapy, e.g., first, second, third and/or fourth fractionated doses of radiotherapy, are administered 30 days prior to administration of a CAR-expressing cell therapy. In some embodiments, the one or more fractionated doses of radiotherapy, e.g., first, second, third and/or fourth fractionated doses of radiotherapy, are administered within 30 days prior to administration of a CAR-expressing cell therapy.

In some embodiments, the one or more fractionated doses of radiotherapy, e.g., first and second fractionated doses of radiotherapy, are administered within about 30 days of each other, e.g., the second dose is administered in less than 30 days from the administration of the first dose.

In some embodiments, the one or more fractionated doses of radiotherapy, e.g., first and second fractionated doses of radiotherapy, are administered more than about 30 days apart, e.g., the second dose is administered more than 30 days, e.g., from the administration of the first dose.

In some embodiments of any of the methods disclosed herein, the lymphodepleting therapy consists of, e.g., consists essentially of radiotherapy.

In some embodiments of any of the methods disclosed herein, the lymphodepleting therapy comprises radiotherapy. In some embodiments, the lymphodepleting therapy further comprises a chemotherapeutic agent described herein. In some embodiments, when the lymphodepleting therapy comprises radiotherapy and a chemotherapeutic agent, the chemotherapeutic agent is not cyclophosphamide.

In some embodiments of any of the methods disclosed herein, the lymphodepleting therapy comprises radiotherapy and a chemotherapeutic agent. In some embodiments, the chemotherapeutic agent is cyclophosphamide. In some embodiments, the cyclophosphamide is administered at a dose of more than 750 mg/m², e.g., about 800, 850, 900, 950, 1000, 1100, 1500 or 2000 mg/m². In some embodiments, the cyclophosphamide is administered at a dose of less than 750 mg/m², e.g., about 700, 650, 600, 550, 500, 400, 300, 200 or 100 mg/m². In some embodiments, the chemotherapeutic agent is cyclophosphamide and the cyclophosphamide is not administered at a does of 750 mg/m².

In some embodiments of a lymphodepleting therapy comprising radiotherapy and cyclophosphamide, the radiotherapy is not administered at a dose of 4 Gy or 2.2 Gy.

In an aspect, provided herein is a method of treating a subject comprising administering to the subject a CAR-expressing cell therapy, e.g., a CAR19 expressing cell therapy, wherein the CAR-expressing cell therapy is administered after stem cell therapy (SCT), e.g., autologous SCT or allogeneic SCT, wherein the subject has not responded, e.g., relapsed, to the SCT, thereby treating the subject.

In some embodiments, the CAR-expressing cell therapy is administered after relapse from SCT, e.g., about 1-12 months, e.g., about 1-3, 3-6, 6-9 or 9-12 months, after relapse. In some embodiments, the CAR-expressing cell therapy is administered after relapse from SCT, e.g., about 1-6 months (e.g., about 1.1-1.5, 1.5-2.0, 2.0-2.5, 2.5-3, 3-3.5, 3.5-4, 4-4.5. 4.5-5, 5-5.5, or 5.5-6 months) after relapse.

In some embodiments, the subject has a response, e.g., remission, a complete response, or a partial response, to the CAR-expressing cell therapy. In some embodiments, the subject in remission has a minimal residual disease (MRD) negative remission, e.g., MRD negative bone marrow remission. In some embodiments, remission is assessed, e.g., determined, by evaluating MRD in a sample, e.g., cerebral spinal fluid or bone marrow, from the subject.

In some embodiments, no response to, or relapse from SCT therapy is determined by evaluating the presence, e.g., reappearance, of cancer cells in the subject, e.g., in the blood or bone marrow. In some embodiments, the presence, e.g., reappearance, of cancer cells comprises detection of the cancer cells at or above a threshold, e.g., above 20%, 15%, 10%, 5%, 4%, 3%, 2%, or 1%.

In some embodiments, the stem cell therapy comprises allogeneic SCT (alloSCT). In some embodiments, the subject receiving alloSCT is a pediatric subject e.g., aged about 18 years of age or younger (e.g., 18, 17, 16, 15, 14, 13, 12, 11, 10, 9, 8, 7, 6, 5, 4, 3, 2, 1 or younger (e.g., 12 months, 6 months, 3 months or less)). In some embodiments, the subject has a leukemia, e.g., ALL, e.g., B cell ALL.

In some embodiments, the stem cell therapy comprises allogeneic SCT (alloSCT). In some embodiments, the subject receiving alloSCT is an adolescent, e.g., aged about 10-19 years, e.g., about 10, 11, 12, 13, 14, 15, 16, 17, 18 or 19 years). In some embodiments, the subject has a leukemia, e.g., ALL, e.g., B cell ALL.

In some embodiments, the stem cell therapy comprises allogeneic SCT (alloSCT). In some embodiments, the subject receiving alloSCT is a young adult (e.g., aged about 18-35 years). In some embodiments, the subject has a leukemia, e.g., ALL, e.g., B cell ALL.

In some embodiments, the SCT comprises autologous SCT.

In some embodiments, the SCT is administered as a first-line therapy, second-line therapy, third line therapy or fourth line therapy. In some embodiments, the SCT is administered as a first line therapy. In some embodiments, the SCT is administered as a second line therapy.

In some embodiments, the SCT, e.g., alloSCT, is administered to the subject when the subject is in first complete remission (CR1), e.g., CR1 after alloSCT.

In some embodiments, the SCT, e.g., alloSCT, is administered to the subject when the subject is in relapse, e.g., first relapse, after SCT, e.g., alloSCT.

In some embodiments, the subject is in relapse, e.g., a first relapse, a second relapse, a third relapse, a fourth relapse or a fifth relapse.

In some embodiments, the subject has previously administered a chemotherapy, e.g., as described herein.

In some embodiments, the subject is a pediatric subject, an adolescent or a young adult. In some embodiments the subject is a pediatric subject, e.g., aged about 18 years of age or younger (e.g., 18, 17, 16, 15, 14, 13, 12, 11, 10, 9, 8, 7, 6, 5, 4, 3, 2, 1 or younger (e.g., 12 months, 6 months, 3 months or less)). In some embodiments, the subject is an adolescent, e.g., aged about 10-19 years, e.g., about 10, 11, 12, 13, 14, 15, 16, 17, 18 or 19 years). In some embodiments, the subject is a young adult (e.g., aged about 18-35 years). In some embodiments, the subject has a hematological cancer, e.g., as described herein. In some embodiments, the subject has a leukemia or a lymphoma. In some embodiments, the subject has a relapsed and/or refractory leukemia or lymphoma. In some embodiments, the subject has ALL, e.g., B cell ALL, e.g., relapsed and/or refractory B-cell ALL. In some embodiments, the subject has NHL, e.g., relapsed and/or refractory NHL.

Additional components of CAR-expressing cells, and methods pertaining to the invention are described below.

In one aspect, disclosed herein is a method of treating a subject having a hematological cancer, comprising administering to the subject in need thereof a plurality of cells that express a chimeric antigen receptor (CAR) molecule. In embodiments, the CAR molecule comprises an antigen binding domain that binds to a tumor antigen selected from a group consisting of: TSHR, CD19, CD123, CD22, CD30, CD171, CS-1, CLL-1, CD33, EGFRvIII, GD2, GD3, BCMA, Tn Ag, PSMA, ROR1, FLT3, FAP, TAG72, CD38, CD44v6, CEA, EPCAM, B7H3, KIT, IL-13Ra2, Mesothelin, IL-11Ra, PSCA, PRSS21, VEGFR2, LewisY, CD24, PDGFR-beta, SSEA-4, CD20, Folate receptor alpha, ERBB2 (Her2/neu), MUC1, EGFR, NCAM, Prostase, PAP, ELF2M, Ephrin B2, IGF-I receptor, CAIX, LMP2, gp100, bcr-abl, tyrosinase, EphA2, Fucosyl GM1, sLe, GM3, TGS5, HMWMAA, o-acetyl-GD2, Folate receptor beta, TEM1/CD248, TEM7R, CLDN6, GPRC5D, CXORF61, CD97, CD179a, ALK, Polysialic acid, PLAC1, GloboH, NY-BR-1, UPK2, HAVCR1, ADRB3, PANX3, GPR20, LY6K, OR51E2, TARP, WT1, NY-ESO-1, LAGE-1a, MAGE-A1, legumain, HPV E6,E7, MAGE A1, ETV6-AML, sperm protein 17, XAGE1, Tie 2, MAD-CT-1, MAD-CT-2, Fos-related antigen 1, p53, p53 mutant, prostein, survivin and telomerase, PCTA-1/Galectin 8, MelanA/MART1, Ras mutant, hTERT, sarcoma translocation breakpoints, ML-IAP, ERG (TMPRSS2 ETS fusion gene), NA17, PAX3, Androgen receptor, Cyclin B1, MYCN, RhoC, TRP-2, CYP1B1, BORIS, SART3, PAX5, OY-TES1, LCK, AKAP-4, SSX2, RAGE-1, human telomerase reverse transcriptase, RU1, RU2, intestinal carboxyl esterase, mut hsp70-2, CD79a, CD79b, CD72, LAIR1, FCAR, LILRA2, CD300LF, CLEC12A, BST2, EMR2, LY75, GPC3, FCRL5, and IGLL1. In some embodiments the CAR molecule binds to CD19.

In embodiments, the subject is a mammal, e.g., a human.

In some embodiments, the subject has a hyperproliferative disorder, e.g., a cancer, e.g., a hematological cancer or a solid tumor. In some embodiments, the subject has a hematological cancer, e.g., a leukemia or a lymphoma. In some embodiments, the hematological cancer is chosen from acute leukemia, B-cell acute lymphoid leukemia (B-ALL), T-cell acute lymphoid leukemia (T-ALL), small lymphocytic leukemia (SLL), acute lymphoid leukemia (ALL), chronic leukemia, chronic myelogenous leukemia (CML), chronic lymphocytic leukemia (CLL), B cell promyelocytic leukemia, blastic plasmacytoid dendritic cell neoplasm, Burkitt's lymphoma, diffuse large B cell lymphoma, follicular lymphoma, hairy cell leukemia, small cell- or a large cell-follicular lymphoma, malignant lymphoproliferative conditions, MALT lymphoma, mantle cell lymphoma (MCL), marginal zone lymphoma, multiple myeloma, myelodysplasia and myelodysplastic syndrome, non-Hodgkin lymphoma (NHL) (e.g., relapsed/refractory NHL), Hodgkin's lymphoma (HL), multiple myeloma, plasmablastic lymphoma, plasmacytoid dendritic cell neoplasm, or Waldenstrom macroglobulinemia. In some embodiments, the hematological cancer is acute lymphoid leukemia (ALL), e.g., B-cell ALL, e.g., relapsed or refractory B-cell ALL. In some embodiments, the hematological cancer is NHL, e.g., relapsed or refractory NHL.

In some embodiments, the CAR-expressing cell therapy, e.g., CAR19 expressing cell therapy, comprises a plurality of cells. In some embodiments, the CAR-expressing cell therapy, e.g., CAR19 expressing cell therapy, is administered in a single infusion or a split-dose infusion. In some embodiments, the CAR-expressing cell therapy, e.g., CAR19 expressing cell therapy, is administered in a single infusion. In some embodiments, the CAR19-expressing cell therapy is administered at a dosage of about 1×10⁸, 2×10⁸, 3×10⁸, 4×10⁸, 5×10⁸, 6×10⁸, 7×10⁸, 8×10⁸, 9×10⁸ cells, e.g., about 5×10⁸ cells, e.g., about 5×10⁸ cells in a single infusion. In some embodiments (e.g., when treating DLBCL, e.g., relapsed or refractory DLBCL), the CAR19-expressing cell therapy is administered at a dosage of about 6×10⁸ cells, e.g., about 6×10⁸ cells in a single infusion. In some embodiments (e.g., when treating DLBCL or ALL, e.g., relapsed or refractory DLBCL or ALL), the CAR19-expressing cell therapy is administered at a dosage of about 0.1, 0.2, 0.3, 0.4, 0.5, 0.6, 0.7, 0.8, or 0.9×10⁸ cells in a single infusion.

In some embodiments of any of the methods disclosed herein, the CAR19-expressing cell therapy comprises a cell (e.g., a population of cells) expressing a murine CAR molecule that binds to CD19 comprising:
(i) one or more of (e.g., all three of) heavy chain complementary determining region 1 (HCDR1), HCDR2, and HCDR3 of any CD19 scFv domain amino acid sequence listed in Table 3 and one or more of (e.g., all three of) light chain complementary determining region 1 (LCDR1), LCDR2, and LCDR3 of any CD19 scFv domain amino acid sequence listed in Table 3,
(ii) a heavy chain variable region (VH) of any CD19 scFv domain amino acid sequence listed in Table 3 and a light chain variable region (VL) of any CD19 scFv domain amino acid sequence listed in Table 3,
(iii) a CD19 scFv domain amino acid sequence listed in Table 3 (e.g., SEQ ID NO: 59, 109, 111, or 114), or
(iv) a full-length CD19 CAR amino acid sequence listed in Table 3 (e.g., SEQ ID NO: 110, 112, 113, or 115, or residues 22-486 of SEQ ID NO: 58)

In some embodiments, the CAR19-expressing cell therapy comprises a cell (e.g., a population of cells) expressing a humanized CAR molecule that binds to CD19 comprising:
(i) one or more of (e.g., all three of) heavy chain complementary determining region 1 (HCDR1), HCDR2, and HCDR3 of any CD19 scFv domain amino acid sequence listed in Table 2 and one or more of (e.g., all three of) light chain complementary determining region 1 (LCDR1), LCDR2, and LCDR3 of any CD19 scFv domain amino acid sequence listed in Table 2,
(ii) a heavy chain variable region (VH) of any CD19 scFv domain amino acid sequence listed in Table 2 and a light chain variable region (VL) of any CD19 scFv domain amino acid sequence listed in Table 2,
(iii) a CD19 scFv domain amino acid sequence listed in Table 2 (e.g., any one of SEQ ID NOs: 1-12), or
(iv) a full-length CD19 CAR amino acid sequence listed in Table 2 (e.g., residues 22-486 of any one of SEQ ID NOs: 31-34 or 42, or residues 22-491 of any one of SEQ ID NOs: 35-41)

In some embodiments, the CAR molecule comprises:
(i) a scFv;
(ii) a transmembrane domain that comprises a transmembrane domain of a protein selected from the group consisting of the alpha, beta or zeta chain of the T-cell receptor, CD28, CD3 epsilon, CD45, CD4, CD5, CD8, CD9, CD16, CD22, CD33, CD37, CD64, CD80, CD86, CD134, CD137 and CD154;
(iii) a hinge region comprising SEQ ID NO:14, or a sequence with 95-99% identity thereof;
(iv) a costimulatory domain that is a functional signaling domain obtained from a protein selected from the group consisting of OX40, CD2, CD27, CD28, CD5, ICAM-1, LFA-1 (CD11a/CD18), ICOS (CD278), and 4-1BB (CD137), wherein optionally the costimulatory domain comprises the amino acid sequence of SEQ ID NO:16 or 51;
(v) an intracellular signaling domain comprising a functional signaling domain of 4-1BB and/or a functional signaling domain of CD3 zeta; e.g., an intracellular signaling domain comprising the sequence of SEQ ID NO: 16 and/or the sequence of SEQ ID NO:17 or 43; or
(vi) a leader sequence, optionally wherein the leader sequence comprises the amino acid sequence of SEQ ID NO: 13.

In some embodiments, the cell comprising a CAR comprises a nucleic acid encoding the CAR. In some embodiments, the nucleic acid encoding the CAR is a lentiviral vector. In some embodiments, the nucleic acid encoding the CAR is introduced into the cells by lentiviral transduction. In some embodiments, the nucleic acid encoding the CAR is an RNA, e.g., an in vitro transcribed RNA. In some embodiments, the nucleic acid encoding the CAR is introduced into the cells by electroporation. In some embodiments,

In some embodiments, the cell, e.g., plurality of cells, comprise T cells or NK cells. In some embodiments, the T cell comprises an autologous T cell or allogeneic T cell.

In embodiments, the subject undergoes lymphodepletion, e.g., as described herein (e.g., with fludarabine, cyclophosphamide, or bendamustine or a combination thereof (e.g., fludarabine and cyclophosphamide, e.g., as described herein) before administration of the immune effector cells.

In embodiments, after administration, the subject experiences complete response (CR), e.g., at day 28 after the administration. In embodiments, the subject has <0.01% minimal residual disease (MRD) (e.g., by flow cytometry), e.g., at day 28 after the administration or 3 months after the administration, e.g., without further anticancer therapy. In embodiments, the subject has CR with MRD (e.g., >0.01%), e.g., at day 28 after the administration or 3 months after the administration, e.g., without further anticancer therapy. In embodiments, after the administration, the subject has no CNS involvement. In embodiments, after the administration, the subject experiences a reduction in CNS status, e.g., from CNS3 to CNS2 or CNS1, or from CNS2 to CNS1. In embodiments, a subject having CNS1 has no detectable blast cells in CSF, a subject having CNS2 has <5 WBC/μl CSF with blast cells; and a subject having CNS3 has □5 WBC/μl CSF with blast cells. In embodiments, the subject is in CR at least at 8, 23, or 31 months after the administration, or at least at 2, 4, 6, 8, 12, 18, 24, 30, or 36 months after the administration. In embodiments, the subject experiences CR for a duration of at least 8, 23, or 31 months after the administration, or at least 2, 4, 6, 8, 12, 18, 24, 30, or 36 months after the administration.

In embodiments, the method further comprises testing a subject for CNS involvement, e.g., by lumbar puncture and/or by imaging to detect brain or ocular involvement, before or after the administration. In embodiments, the method further comprises testing a subject for bone marrow disease or MRD, before or after the administration. In embodiments, the testing is performed at one or more of 1, 3, 6, 9, or 12 months after the administration.

In embodiments, after the administration, the subject does not experience one or more of: CRS, severe CRS, encephalopathy (e.g., encephalopathy grade 2-3), seizures (e.g., seizures grade 2-4), vision disturbance, speech disturbance, trigeminal neuralgia, confusion, dizziness, ataxia, or agitation.

In some embodiments, the immune effector cell is an immune effector cell described herein. In some embodiments, the CAR molecule is a CAR molecule described herein. In some embodiments, the CAR molecule comprises the amino acid sequence of residues 22-486 of SEQ ID NO: 58, residues 22-486 of any one of SEQ ID NOs: 31-34 or 42, or residues 22-491 of any one of SEQ ID NOs: 35-41. In some embodiments the CAR molecule comprises an antigen binding domain comprising one or more sequence selected from SEQ ID NOS:1-12. In embodiments, the immune effector cells are administered as a monotherapy.

In some aspects, the present disclosure also provides a method of treating a human subject (e.g., a pediatric or young adult subject) having acute lymphoid leukemia (ALL), comprising: administering to the subject immune effector cells expressing a CAR molecule that binds to CD19, wherein said CAR molecule comprises the amino acid sequence of residues 22-486 of SEQ ID NO: 58, residues 22-486 of any one of SEQ ID NOs: 31-34 or 42, or residues 22-491 of any one of SEQ ID NOs: 35-41, at a dose of 2.0-5.0×10⁶ cells/kg.

In embodiments, the subject experiences remission (e.g., CR or CRi) after the administration of the immune effector cells. In embodiments, the subject is treated with lymphodepleting therapy, e.g., as described herein, before the administration of the immune effector cells.

In embodiments, the dose of immune effector cells is about 2.0-3.0×10⁶, 2.0-4.0×10⁶, 2.0-5.0×10⁶, 3.0-4.0×10⁶, 3.0-5.0×10⁶, or 4.0-5.0×10⁶ cells/kg. In embodiments, the dose of immune effector cells is about 2.0×10⁶, 3.0×10⁶, or 4.0×10⁶ cells/kg. In embodiments, the dose of immune effector cells is about 1.0-1.5×10⁸, 1.0-2.0×10⁸, 1.0-2.5×10⁸, 1.5-2.0×10⁸, 1.5-2.5×10⁸, or 2.0-2.5×10⁸ cells. In embodiments, the dose of immune effector cells is about 1.0×10⁸, 1.5×10⁸, or 2.0-2.5×10⁸ cells. In embodiments, the subject receives a single dose of cells. In embodiments, the subject weighs ≤50 kg. In embodiments, the subject weighs >50 kg.

In embodiments of any of the preceding methods, the hematological cancer is a B cell malignancy, e.g., chosen from multiple myeloma, chronic lymphocytic leukemia, acute lymphoblastic leukemia (ALL), or non-Hodgkins lymphoma. In some embodiments, the hematological cancer is ALL, e.g., B-ALL. In some embodiments, the hematological cancer is non-Hodgkin lymphoma (NHL), e.g., relapsed/refractory NHL.

In some aspects, the present disclosure provides a method of evaluating a subject, e.g., evaluating or monitoring CRS status (e.g., the risk or level of CRS) or the effectiveness of a CAR-expressing cell therapy in a subject, having a cancer.

In embodiments, the CAR-expressing cell therapy is a CAR19-expressing cell therapy, e.g., for B-ALL, or NHL (e.g., relapsed/refractory NHL).

In embodiments, the CAR-expressing cell therapy comprises a plurality of CAR-expressing immune effector cells. In embodiments, the CAR-expressing cell therapy is a CAR19 therapy (e.g., CTL019 therapy).

In embodiments, the subject is evaluated prior to, during, or after receiving the CAR-expressing cell therapy.

In some aspects, the present disclosure provides a method of evaluating a subject, e.g., evaluating or monitoring the effectiveness of a CAR-expressing cell therapy (e.g., CD19 CAR, e.g., CTL019) in a subject, having a cancer, comprising acquiring a value of a CAR-expressing cell therapy pharmacokinetic measure in the subject, wherein the pharmacokinetic measure is selected from:
a) peak expansion of CAR-expressing cells, e.g., wherein a peak expansion of over about 3, 3.5, 4, 4.5, or 5 (and optionally up to 6) log₁₀ CAR copies/μg genomic DNA is indicative of response, e.g., CR, PR_{TD}, or PR;
b) persistence of CAR-expressing cells, e.g., wherein an AUC of over about 300, 350, 400, 450, or 500 (and optionally up to 600 or 700) log₁₀ CAR copies/μg genomic DNA over time (e.g., over 12 months) is indicative of response, e.g., CR, PR_{TD}, or PR; or
c) in vitro proliferation of CAR-expressing cells, e.g., wherein a CAR-expressing cell fold-expansion of over about 25, 30, 35, 40, 45, 50, 60, 70, 80, 90, or 100 (and optionally up to 100 or 150) fold expansion is indicative of CR, PR_{TD};
wherein said value is indicative of the subject's responsiveness or relapsing status to the CAR-expressing cell therapy, thereby evaluating the subject.

In some aspects, the present disclosure provides a method of evaluating a subject, e.g., evaluating or monitoring the effectiveness of a CAR-expressing cell therapy in a subject, having a cancer, comprising acquiring a value of a pro-apoptotic signalling molecule level or activity in the subject, wherein said value is indicative of the subject's responsiveness or relapsing status to the CAR-expressing cell therapy, thereby evaluating the subject.

### CAR Molecules

In certain embodiments, the method of treatment comprises a CAR therapy, e.g., administration of one or more cells that express one or more CAR molecules. A cell expressing one or more CAR molecules can be an immune effector cell, e.g., a T cell or NK cell. In an embodiment, the subject is a human.

In one embodiment, the cell expressing the CAR molecule comprises a vector that includes a nucleic acid sequence encoding the CAR molecule. In one embodiment, the vector is selected from the group consisting of a DNA, an RNA, a plasmid, a lentivirus vector, adenoviral vector, or a retrovirus vector. In one embodiment, the vector is a lentivirus vector. In one embodiment, the vector further comprises a promoter. In one embodiment, the promoter is an EF-1 promoter. In one embodiment, the EF-1 promoter comprises a sequence of SEQ ID NO: 100. In one embodiment, the vector is an in vitro transcribed vector, e.g., a vector that transcribes RNA of a nucleic acid molecule described herein. In one embodiment, the nucleic acid sequence in the in vitro vector further comprises a poly(A) tail, e.g., a poly A tail described herein, e.g., comprising about 150 adenosine bases. In one embodiment, the nucleic acid sequence in the in vitro vector further comprises a 3′UTR, e.g., a 3′ UTR described herein, e.g., comprising at least one repeat of a 3′UTR derived from human beta-globulin. In one embodiment, the nucleic acid sequence in the in vitro vector further comprises promoter. In one embodiment, the nucleic acid sequence comprises a T2A sequence.

In one embodiment, the cell expressing the CAR molecule is a cell described herein, e.g., a human T cell or a human NK cell, e.g., a human T cell described herein or a human NK cell described herein. In one embodiment, the human T cell is a CD8+ T cell. In one embodiment, the human T cell is a CD4+ T cell. In one embodiment, the human T cell is a CD4+/CD8+ T cell. In one embodiment the human T cell is a mixture of CD8+ and CD4+ T cells. In one embodiment, the cell is an autologous T cell. In one embodiment, the cell is an allogeneic T cell. In one embodiment, the cell is a T cell and the T cell is diacylglycerol kinase (DGK) deficient. In one embodiment, the cell is a T cell and the T cell is Ikaros deficient. In one embodiment, the cell is a T cell and the T cell is both DGK and Ikaros deficient.

In another embodiment, the cell expressing the CAR molecule, e.g., as described herein, can further express another agent, e.g., an agent which enhances the activity of a CAR-expressing cell.

In one embodiment, the method includes administering a cell expressing the CAR molecule, as described herein, in combination with an agent which enhances the activity of a CAR-expressing cell, wherein the agent is a cytokine, e.g., IL-7, IL-15, IL-21, or a combination thereof. The cytokine can be delivered in combination with, e.g., simultaneously or shortly after, administration of the CAR-expressing cell. Alternatively, the cytokine can be delivered after a prolonged period of time after administration of the CAR-expressing cell, e.g., after assessment of the subject's response to the CAR-expressing cell.

For example, in one embodiment, the agent that enhances the activity of a CAR-expressing cell can be an agent which inhibits an immune inhibitory molecule. Examples of immune inhibitory molecules include PD1, PD-L1, CTLA4, TIM3, CEACAM (e.g., CEACAM-1, CEACAM-3 and/or CEACAM-5), LAG3, VISTA, BTLA, TIGIT, LAIR1, CD160, 2B4 and TGF beta. In one embodiment, the agent that inhibits an immune inhibitory molecule comprises a first polypeptide, e.g., an inhibitory molecule, associated with a second polypeptide that provides a positive signal to the cell, e.g., an intracellular signaling domain described herein. In one embodiment, the agent comprises a first polypeptide, e.g., of an immune inhibitory molecule such as PD1, PD-L1, CTLA4, TIM3, CEACAM (e.g., CEACAM-1, CEACAM-3 and/or CEACAM-5), LAG3, VISTA, BTLA, TIGIT, LAIR1, CD160, 2B4 or TGF beta, or a fragment of any of these (e.g., at least a portion of the extracellular domain of any of these), and a second polypeptide which is an intracellular signaling domain described herein (e.g., comprising a costimulatory domain (e.g., 41BB, CD27 or CD28, e.g., as described herein) and/or a primary signaling domain (e.g., a CD3 zeta signaling domain described herein). In one embodiment, the agent comprises a first polypeptide of PD1 or a fragment thereof (e.g., at least a portion of the extracellular domain of PD1), and a second polypeptide of an intracellular signaling domain described herein (e.g., a CD28 signaling domain described herein and/or a CD3 zeta signaling domain described herein).

In one embodiment, lymphocyte infusion, for example allogeneic lymphocyte infusion, is used in the treatment of the cancer, wherein the lymphocyte infusion comprises at least one CD19 CAR-expressing cell described herein and optionally at least one cell expressing a CAR directed against a B-cell antigen. In one embodiment, autologous lymphocyte infusion is used in the treatment of the cancer, wherein the autologous lymphocyte infusion comprises at least one CD19-expressing cell, and optionally at least one cell expressing a CAR directed against a B-cell antigen.

In one embodiment, the CAR expressing cell, e.g., T cell, is administered to a subject that has received a previous stem cell transplantation, e.g., autologous stem cell transplantation or allogenenic stem cell transplantation, or a subject that has received a previous dose of melphalan.

In one embodiment, the cell expressing the CAR molecule, e.g., a CAR molecule described herein, is administered in combination with an agent that ameliorates one or more side effect associated with administration of a cell expressing a CAR molecule or with administration of the B-cell inhibitor, e.g., an agent described herein.

In one embodiment, the cell expressing the CAR molecule, e.g., a CD19 CAR, and the B-cell inhibitor are administered in combination with an additional agent that treats the disease associated with CD19, e.g., an additional agent described herein.

In one embodiment, the cells expressing a CAR molecule, e.g., a CAR molecule described herein, are administered at a dose and/or dosing schedule described herein.

In one embodiment, the CAR molecule is introduced into T cells, e.g., using in vitro transcription, and the subject (e.g., human) receives an initial administration of cells comprising a CAR molecule, and one or more subsequent administrations of cells comprising a CAR molecule, wherein the one or more subsequent administrations are administered less than 15 days, e.g., 14, 13, 12, 11, 10, 9, 8, 7, 6, 5, 4, 3, or 2 days after the previous administration. In one embodiment, more than one administration of cells comprising a CAR molecule are administered to the subject (e.g., human) per week, e.g., 2, 3, or 4 administrations of cells comprising a CAR molecule are administered per week. In one embodiment, the subject (e.g., human subject) receives more than one administration of cells comprising a CAR molecule per week (e.g., 2, 3 or 4 administrations per week) (also referred to herein as a cycle), followed by a week of no administration of cells comprising a CAR molecule, and then one or more additional administration of cells comprising a CAR molecule (e.g., more than one administration of the cells comprising a CAR molecule per week) is administered to the subject. In another embodiment, the subject (e.g., human subject) receives more than one cycle of cells comprising a CAR molecule, and the time between each cycle is less than 10, 9, 8, 7, 6, 5, 4, or 3 days. In one embodiment, the cells comprising a CAR molecule are administered every other day for 3 administrations per week. In one embodiment, the cells comprising a CAR molecule are administered for at least two, three, four, five, six, seven, eight or more weeks.

In one embodiment, a population of cells described herein is administered. In some embodiments the population of cells is isolated or purified.

In one embodiment, the 4-1BB costimulatory domain comprises a sequence of SEQ ID NO: 16. In one embodiment, the 4-1BB costimulatory domain comprises an amino acid sequence having at least one, two or three modifications (e.g., substitutions) but not more than 20, 10 or 5 modifications (e.g., substitutions) of an amino acid sequence of SEQ ID NO: 16, or a sequence with at least 95%, e.g., 95-99%, identity to an amino acid sequence of SEQ ID NO:16. In one embodiment, the 4-1BB costimulatory domain is encoded by a nucleic acid sequence of SEQ ID NO:60, or a sequence with at least 95%, e.g., 95-99%, identity thereof.

In one embodiment, the CD27 costimulatory domain comprises a sequence of SEQ ID NO: 16. In one embodiment, the CD27 costimulatory domain comprises an amino acid sequence having at least one, two or three modifications (e.g., substitutions) but not more than 20, 10 or 5 modifications (e.g., substitutions) of an amino acid sequence of SEQ ID NO: 16, or a sequence with 95-99% identity to an amino acid sequence of SEQ ID NO:16. In one embodiment, the CD27 costimulatory domain is encoded by a nucleic acid sequence of SEQ ID NO:17, or a sequence with at least 95%, e.g., 95-99%, identity thereof.

In one embodiment, the CD28 costimulatory domain comprises a sequence of SEQ ID NO: 1317. In one embodiment, the CD28 costimulatory domain comprises an amino acid sequence having at least one, two or three modifications (e.g., substitutions) but not more than 20, 10 or 5 modifications (e.g., substitutions) of an amino acid sequence of SEQ ID NO: 1317, or a sequence with at least 95%, e.g., 95-99%, identity to an amino acid sequence of SEQ ID NO:1317. In one embodiment, the CD28 costimulatory domain is encoded by a nucleic acid sequence of SEQ ID NO:1318, or a sequence with at least 95%, e.g., 95-99%, identity thereof.

In one embodiment, the wild-type ICOS costimulatory domain comprises a sequence of SEQ ID NO: 1319. In one embodiment, the wild-type ICOS costimulatory domain comprises an amino acid sequence having at least one, two or three modifications (e.g., substitutions) but not more than 20, 10 or 5 modifications (e.g., substitutions) of an amino acid sequence of SEQ ID NO: 1319, or a sequence with at least 95%, e.g., 95-99%, identity to an amino acid sequence of SEQ ID NO: 1319. In one embodiment, the wild-type ICOS costimulatory domain is encoded by a nucleic acid sequence of SEQ ID NO: 1320, or a sequence with at least 95%, e.g., 95-99%, identity thereof.

In one embodiment, the Y to F mutant ICOS costimulatory domain comprises a sequence of SEQ ID NO: 1321. In one embodiment, the Y to F mutant ICOS costimulatory domain comprises an amino acid sequence having at least one, two or three modifications (e.g., substitutions) but not more than 20, 10 or 5 modifications (e.g., substitutions) of an amino acid sequence of SEQ ID NO: 1321, or a sequence with at least 95%, e.g., 95-99%, identity to an amino acid sequence of SEQ ID NO: 1321. In one embodiment, the Y to F mutant ICOS costimulatory domain is encoded by a nucleic acid sequence with at least 95%, e.g., 95-99%, identity to a nucleic acid sequence of SEQ ID NO:1320 (wherein SEQ ID NO: 1320 encodes wild-type ICOS).

In embodiments, the primary signaling domain comprises a functional signaling domain of CD3 zeta. In embodiments, the functional signaling domain of CD3 zeta comprises SEQ ID NO: 17 (mutant CD3 zeta) or SEQ ID NO: 43 (wild-type human CD3 zeta).

In one embodiment, the method includes administering a population of cells wherein at least one cell in the population expresses a CAR, e.g., having an anti-CD19 domain described herein, and an agent which enhances the activity of a CAR-expressing cell, e.g., a second cell expressing the agent which enhances the activity of a CAR-expressing cell. For example, in one embodiment, the agent can be an agent which inhibits an immune inhibitory molecule. Examples of immune inhibitory molecules include PD1, PD-L1, CTLA4, TIM3, CEACAM (e.g., CEACAM-1, CEACAM-3 and/or CEACAM-5), LAG3, VISTA, BTLA, TIGIT, LAIR1, CD160, 2B4 and TGF beta. In one embodiment, the agent that inhibits an immune inhibitory molecule comprises a first polypeptide, e.g., an inhibitory molecule, associated with a second polypeptide that provides a positive signal to the cell, e.g., an intracellular signaling domain described herein. In one embodiment, the agent comprises a first polypeptide, e.g., of an inhibitory molecule such as PD1, PD-L1, CTLA4, TIM3, CEACAM (e.g., CEACAM-1, CEACAM-3 and/or CEACAM-5), LAG3, VISTA, BTLA, TIGIT, LAIR1, CD160, 2B4 or TGF beta, or a fragment of any of these (e.g., at least a portion of an extracellular domain of any of these), and a second polypeptide which is an intracellular signaling domain described herein (e.g., comprising a costimulatory domain (e.g., 41BB, CD27 or CD28, e.g., as described herein) and/or a primary signaling domain (e.g., a CD3 zeta signaling domain described herein). In one embodiment, the agent comprises a first polypeptide of PD1 or a fragment thereof (e.g., at least a portion of the extracellular domain of PD1), and a second polypeptide of an intracellular signaling domain described herein (e.g., a CD28 signaling domain described herein and/or a CD3 zeta signaling domain described herein).

In an embodiment, the method further comprises transplanting a cell, e.g., a hematopoietic stem cell, or a bone marrow, into the mammal.

In one embodiment, the method includes administering a population of cells comprising a CAR described herein, e.g., a CAR having an anti-CD19 domain described herein, and an agent which enhances the activity of a CAR-expressing cell, wherein the agent is a cytokine, e.g., IL-7; IL-15 (e.g., an IL-15 polypeptide); an IL-15 receptor alpha (IL-15Ra) polypeptide; a combination of both a IL-15 polypeptide and a IL-15Ra polypeptide (e.g., hetIL-15); or IL-21, or a combination thereof. The cytokine can be delivered in combination with, e.g., simultaneously or shortly after, administration of the CAR-expressing cell(s). Alternatively, the cytokine can be delivered after a prolonged period of time after administration of the CAR-expressing cell(s), e.g., after assessment of the subject's response to the CAR-expressing cell(s). Related compositions for use and methods of making a medicament are also provided.

In an embodiment, the composition is a pharmaceutically acceptable composition.

In some embodiment, the CAR molecules described herein include a binding domain, e.g., a CD19-binding domain as described herein.

In one embodiment, the CAR molecule comprises a transmembrane domain of a protein selected from the group consisting of the alpha, beta or zeta chain of the T-cell receptor, CD28, CD3 epsilon, CD45, CD4, CD5, CD8, CD9, CD16, CD22, CD33, CD37, CD64, CD80, CD86, CD134, CD137 and CD154. In one embodiment, the transmembrane domain comprises a sequence of SEQ ID NO: 15. In one embodiment, the transmembrane domain comprises an amino acid sequence having at least one, two or three modifications (e.g., substitutions) but not more than 20, 10 or 5 modifications (e.g., substitutions) of an amino acid sequence of SEQ ID NO: 15, or a sequence with 95-99% identity to an amino acid sequence of SEQ ID NO: 15.

In one embodiment, the binding domain is connected to the transmembrane domain by a hinge region, e.g., a hinge region described herein. In one embodiment, the encoded hinge region comprises SEQ ID NO:14 or SEQ ID NO:45, or a sequence with 95-99% identity thereof.

In one embodiment, the CAR molecule further comprises a sequence encoding a costimulatory domain, e.g., a costimulatory domain described herein. In one embodiment, the costimulatory domain comprises a functional signaling domain of a protein selected from the group consisting of OX40, CD2, CD27, CD28, CD5, ICAM-1, LFA-1 (CD11a/CD18), ICOS (CD278), and 4-1BB (CD137). In one embodiment, the costimulatory domain comprises a sequence of SEQ ID NO: 16. In one embodiment, the costimulatory domain comprises a sequence of SEQ ID NO:51. In one embodiment, the costimulatory domain comprises an amino acid sequence having at least one, two or three modifications (e.g., substitutions) but not more than 20, 10 or 5 modifications (e.g., substitutions) of an amino acid sequence of SEQ ID NO: 16 or SEQ ID NO:51, or a sequence with at least 95%, e.g., 95-99%, identity to an amino acid sequence of SEQ ID NO: 16 or SEQ ID NO:51. In one embodiment, the costimulatory domain comprises a functional signaling domain of a protein selected from the group consisting of MHC class I molecule, TNF receptor proteins, Immunoglobulin-like proteins, cytokine receptors, integrins, signaling lymphocytic activation molecules (SLAM proteins), activating NK cell receptors, BTLA, a Toll ligand receptor, OX40, CD2, CD7, CD27, CD28, CD30, CD40, CD5, ICAM-1, LFA-1 (CD11a/CD18), 4-1BB (CD137), B7-H3, CDS, ICAM-1, ICOS (CD278), GITR, BAFFR, LIGHT, HVEM (LIGHTR), KIRDS2, SLAMF7, NKp80 (KLRF1), NKp44, NKp30, NKp46, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, NKG2D, NKG2C, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, and a ligand that specifically binds with CD83. In embodiments, the costimulatory domain comprises 4-1BB, CD27, CD28, or ICOS.

In one embodiment, the CAR molecule further comprises a sequence encoding an intracellular signaling domain, e.g., an intracellular signaling domain described herein. In one embodiment, the intracellular signaling domain comprises a functional signaling domain of 4-1BB and/or a functional signaling domain of CD3 zeta. In one embodiment, the intracellular signaling domain comprises the sequence of SEQ ID NO: 16 and/or the sequence of SEQ ID NO:17. In one embodiment, the intracellular signaling domain comprises the sequence of SEQ ID NO:16 and/or the sequence of SEQ ID NO:43. In one embodiment, the intracellular signaling domain comprises a functional signaling domain of CD27 and/or a functional signaling domain of CD3 zeta. In one embodiment, the intracellular signaling domain comprises the sequence of SEQ ID NO: 51 and/or the sequence of SEQ ID NO:17. In one embodiment, the intracellular signaling domain comprises the sequence of SEQ ID NO:51 and/or the sequence of SEQ ID NO:43. In one embodiment, the intracellular signaling domain comprises an amino acid sequence having at least one, two or three modifications (e.g., substitutions) but not more than 20, 10 or 5 modifications (e.g., substitutions) of an amino acid sequence of SEQ ID NO:16 or SEQ ID NO:51 and/or an amino acid sequence of SEQ ID NO:17 or SEQ ID NO:43, or a sequence with at least 95%, e.g., 95-99%, identity to an amino acid sequence of SEQ ID NO:16 or SEQ ID NO:51 and/or an amino acid sequence of SEQ ID NO:17 or SEQ ID NO:43. In one embodiment, the intracellular signaling domain comprises the sequence of SEQ ID NO:16 or SEQ ID NO:51 and the sequence of SEQ ID NO: 17 or SEQ ID NO:43, wherein the sequences comprising the intracellular signaling domain are expressed in the same frame and as a single polypeptide chain.

In one embodiment, the CAR molecule further comprises a leader sequence, e.g., a leader sequence described herein. In one embodiment, the leader sequence comprises an amino acid sequence of SEQ ID NO: 13, or a sequence with 95-99% identity to an amino acid sequence of SEQ ID NO:13.

In one aspect, the CAR (e.g., a CD19 CAR) comprises an optional leader sequence (e.g., an optional leader sequence described herein), an extracellular antigen binding domain, a hinge (e.g., hinge described herein), a transmembrane domain (e.g., transmembrane domain described herein), and an intracellular stimulatory domain (e.g., intracellular stimulatory domain described herein). In one aspect an exemplary CAR construct comprises an optional leader sequence (e.g., a leader sequence described herein), an extracellular antigen binding domain, a hinge, a transmembrane domain, an intracellular costimulatory domain (e.g., an intracellular costimulatory domain described herein) and an intracellular stimulatory domain.

CAR which comprises a transmembrane domain that comprises a transmembrane domain of a protein selected from the group consisting of the alpha, beta or zeta chain of the T-cell receptor, CD28, CD3 epsilon, CD45, CD4, CD5, CD8, CD9, CD16, CD22, CD33, CD37, CD64, CD80, CD86, CD134, CD137 and CD154. In embodiments, the antigen binding domain is connected to the transmembrane domain by a hinge region. In embodiments, the hinge region comprises SEQ ID NO:14, or a sequence with 95-99% identity thereof. In embodiments, the costimulatory domain is a functional signaling domain obtained from a protein selected from the group consisting of OX40, CD2, CD27, CD28, CD5, ICAM-1, LFA-1 (CD11a/CD18), ICOS (CD278), and 4-1BB (CD137). In embodiments, the costimulatory domain is a functional signaling domain obtained from a protein selected from the group consisting of MHC class I molecule, TNF receptor proteins, Immunoglobulin-like proteins, cytokine receptors, integrins, signaling lymphocytic activation molecules (SLAM proteins), activating NK cell receptors, BTLA, a Toll ligand receptor, OX40, CD2, CD7, CD27, CD28, CD30, CD40, CDS, ICAM-1, LFA-1 (CD11a/CD18), 4-1BB (CD137), B7-H3, CDS, ICAM-1, ICOS (CD278), GITR, BAFFR, LIGHT, HVEM (LIGHTR), KIRDS2, SLAMF7, NKp80 (KLRF1), NKp44, NKp30, NKp46, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, NKG2D, NKG2C, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, and a ligand that specifically binds with CD83. In embodiments, the costimulatory domain comprises a sequence of SEQ ID NO:16 or SEQ ID NO:51. In embodiments, the intracellular signaling domain comprises a functional signaling domain of 4-1BB and/or a functional signaling domain of CD3 zeta.

In embodiments, the intracellular signaling domain comprises the sequence of SEQ ID NO: 16 and/or the sequence of SEQ ID NO:17 or SEQ ID NO:43. In embodiments, the CAR further comprises a leader sequence. In embodiments, the leader sequence comprises SEQ ID NO: 13.

In embodiments, the cells that express the CAR molecule comprise T cells or NK cells.

In embodiments, the compositions disclosed herein (e.g., nucleic acids, vectors, or cells) are for use as a medicament.

In embodiments, the compositions disclosed herein are used in the treatment of a hematological cancer.

In embodiments, the compositions disclosed herein are used in the treatment of a disease associated with expression of a B-cell antigen (e.g., CD19), e.g., a B-cell leukemia or lymphoma (e.g., a CD19-associated disease), e.g., B-cell ALL or NHL (e.g., relapsed or refractory NHL).

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein (e.g., sequence database reference numbers) are incorporated by reference in their entirety. For example, all GenBank, Unigene, and Entrez sequences referred to herein, e.g., in any Table herein, are incorporated by reference. Unless otherwise specified, the sequence accession numbers specified herein, including in any Table herein, refer to the database entries current as of Apr. 8, 2015. When one gene or protein references a plurality of sequence accession numbers, all of the sequence variants are encompassed.

In addition, the materials, methods, and examples are illustrative only and not intended to be limiting.

Headings, sub-headings or numbered or lettered elements, e.g., (a), (b), (i) etc, are presented merely for ease of reading. The use of headings or numbered or lettered elements in this document does not require the steps or elements be performed in alphabetical order or that the steps or elements are necessarily discrete from one another.

Other features, objects, and advantages of the invention will be apparent from the description and drawings, and from the claims.

### BRIEF DESCRIPTIONS OF THE DRAWINGS

FIGS. 1A-1B are graphs depicting patient survival following radiation therapy and CAR19 therapy. The patients were either not administered RT, or treated with remote RT, prior RT or bridging/lymphodepleting RT (e.g., tandem/induction RT). FIG. 1A shows a graph depicting progression-free survival. FIG. 1B shows a graph depicting overall survival.

FIG. 2 is a graph depicting absolute lymphocyte counts (ALC) in subjects receiving tandem/induction RT. ALC levels before RT and after RT are plotted on the x-axis.

### DETAILED DESCRIPTION

### Definitions

Unless defined otherwise, all technical and scientific terms used herein have the same meaning as commonly understood by one of ordinary skill in the art to which the invention pertains.

The term “a” and “an” refers to one or to more than one (i.e., to at least one) of the grammatical object of the article. By way of example, “an element” means one element or more than one element.

The term “about” when referring to a measurable value such as an amount, a temporal duration, and the like, is meant to encompass variations of ±20% or in some instances ±10%, or in some instances ±5%, or in some instances ±1%, or in some instances ±0.1% from the specified value, as such variations are appropriate to perform the disclosed methods.

The term “apheresis” as used herein refers to the art-recognized extracorporeal process by which the blood of a donor or patient is removed from the donor or patient and passed through an apparatus that separates out selected particular constituent(s) and returns the remainder to the circulation of the donor or patient, e.g., by retransfusion. Thus, “an apheresis sample” refers to a sample obtained using apheresis.

The term “bioequivalent” refers to an amount of an agent other than the reference compound (e.g., RAD001), required to produce an effect equivalent to the effect produced by the reference dose or reference amount of the reference compound (e.g., RAD001). In an embodiment the effect is the level of mTOR inhibition, e.g., as measured by P70 S6 kinase inhibition, e.g., as evaluated in an in vivo or in vitro assay, e.g., as measured by an assay described herein, e.g., the Boulay assay, or measurement of phosphorylated S6 levels by western blot. In an embodiment, the effect is alteration of the ratio of PD-1 positive/PD-1 negative T cells, as measured by cell sorting. In an embodiment a bioequivalent amount or dose of an mTOR inhibitor is the amount or dose that achieves the same level of P70 S6 kinase inhibition as does the reference dose or reference amount of a reference compound. In an embodiment, a bioequivalent amount or dose of an mTOR inhibitor is the amount or dose that achieves the same level of alteration in the ratio of PD-1 positive/PD-1 negative T cells as does the reference dose or reference amount of a reference compound.

The term “inhibition” or “inhibitor” includes a reduction in a certain parameter, e.g., an activity, of a given molecule, e.g., CD20, CD19, or BCMA. For example, inhibition of an activity, e.g., an activity of CD19, of at least 5%, 10%, 20%, 30%, 40%, or more is included by this term. Thus, inhibition need not be 100%. Activities for the inhibitors can be determined as described herein or by assays known in the art. A “B-cell inhibitor” is a molecule, e.g., a small molecule, antibody, CAR or cell comprising a CAR, which causes the reduction in a certain parameter, e.g., an activity, e.g., growth or proliferation, of a B-cell, or which causes a reduction in a certain parameter, e.g., an activity, of a molecule associated with a B cell. Non-limiting examples of molecules associated with a B cell include proteins expressed on the surface of B cells, e.g., CD19, CD20, CD10, CD22, CD34, CD123, FLT-3, ROR1, CD79b, CD179b, CD79a, or BCMA.

The term “Chimeric Antigen Receptor” or alternatively a “CAR” refers to a set of polypeptides, typically two in the simplest embodiments, which when in an immune effector cell, provides the cell with specificity for a target cell, typically a cancer cell, and with intracellular signal generation. In some embodiments, a CAR comprises at least an extracellular antigen binding domain, a transmembrane domain and a cytoplasmic signaling domain (also referred to herein as “an intracellular signaling domain”) comprising a functional signaling domain derived from a stimulatory molecule and/or costimulatory molecule as defined below. In some embodiments, the set of polypeptides are in the same polypeptide chain, e.g., comprise a chimeric fusion protein. In some embodiments, the set of polypeptides are not contiguous with each other, e.g., are in different polypeptide chains. In some embodiments, the set of polypeptides include a dimerization switch that, upon the presence of a dimerization molecule, can couple the polypeptides to one another, e.g., can couple an antigen binding domain to an intracellular signaling domain. In one aspect, the stimulatory molecule of the CAR is the zeta chain associated with the T cell receptor complex (e.g., CD3 zeta). In one aspect, the cytoplasmic signaling domain comprises a primary signaling domain (e.g., a primary signaling domain of CD3-zeta).

In one aspect, the cytoplasmic signaling domain further comprises one or more functional signaling domains derived from at least one costimulatory molecule as defined below. In one aspect, the costimulatory molecule is chosen from the costimulatory molecules described herein, e.g., 4-1BB (i.e., CD137), CD27, and/or CD28. In one aspect, the CAR comprises a chimeric fusion protein comprising an extracellular antigen binding domain, a transmembrane domain and an intracellular signaling domain comprising a functional signaling domain derived from a stimulatory molecule. In one aspect, the CAR comprises a chimeric fusion protein comprising an extracellular antigen binding domain, a transmembrane domain and an intracellular signaling domain comprising a functional signaling domain derived from a costimulatory molecule and a functional signaling domain derived from a stimulatory molecule. In one aspect, the CAR comprises a chimeric fusion protein comprising an extracellular antigen binding domain, a transmembrane domain and an intracellular signaling domain comprising two functional signaling domains derived from one or more costimulatory molecule(s) and a functional signaling domain derived from a stimulatory molecule. In one aspect, the CAR comprises a chimeric fusion protein comprising an extracellular antigen binding domain, a transmembrane domain and an intracellular signaling domain comprising at least two functional signaling domains derived from one or more costimulatory molecule(s) and a functional signaling domain derived from a stimulatory molecule. In one aspect the CAR comprises an optional leader sequence at the amino-terminus (N-ter) of the CAR fusion protein. In one aspect, the CAR further comprises a leader sequence at the N-terminus of the extracellular antigen binding domain, wherein the leader sequence is optionally cleaved from the antigen binding domain (e.g., a scFv) during cellular processing and localization of the CAR to the cellular membrane.

As used herein, the term “treatment” refers to an approach for obtaining a beneficial or a desired result including, but not limited to: a therapeutic benefit; or prevention of a condition, e.g., a side effect, e.g., an unwanted effect as described herein. The terms “treatment”, “treating”, and “ameliorating” are used interchangeably herein. In some embodiments, a therapeutic benefit is obtained by eradication or amelioration of the underlying disorder being treated. In some embodiments, a therapeutic benefit is obtained by reduction of, eradication, or amelioration of one or more of the symptoms, e.g., physiological symptoms, associated with the underlying disorder such that an improvement, e.g., change, is observed in the patient. In some embodiments, the patient can still be afflicted with the underlying disorder. In some embodiments, treatment comprises prevention of a condition, e.g., a side effect, e.g., an unwanted side effect from a therapy. Treatment or prevention of a condition or a side effect need not be a complete treatment or prevention of the condition or side effect.

As used herein, unless otherwise specified, the terms “prevent,” “preventing” and “prevention” refer to an action that occurs before the subject begins to suffer from the condition, or relapse of the condition. Prevention need not result in a complete prevention of the condition; partial prevention or reduction of the condition or a symptom of the condition, or reduction of the risk of developing the condition, is encompassed by this term.

Administered “in combination”, as used herein, means that two (or more) different treatments are delivered to the subject during the course of the subject's affliction with the disorder, e.g., the two or more treatments are delivered after the subject has been diagnosed with the disorder and before the disorder has been cured or eliminated or treatment has ceased for other reasons. In some embodiments, the delivery of one treatment is still occurring when the delivery of the second begins, so that there is overlap in terms of administration. This is sometimes referred to herein as “simultaneous” or “concurrent delivery”. In other embodiments, the delivery of one treatment ends before the delivery of the other treatment begins. In some embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In some embodiments, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered. In one embodiment, the CAR-expressing cell is administered at a dose and/or dosing schedule described herein, and the B-cell inhibitor, or agent that enhances the activity of the CD19 CAR-expressing cell is administered at a dose and/or dosing schedule described herein.

“Derived from” as that term is used herein, indicates a relationship between a first and a second molecule. It generally refers to structural similarity between the first molecule and a second molecule and does not connote or include a process or source limitation on a first molecule that is derived from a second molecule. For example, in the case of an intracellular signaling domain that is derived from a CD3zeta molecule, the intracellular signaling domain retains sufficient CD3zeta structure such that is has the required function, namely, the ability to generate a signal under the appropriate conditions. It does not connote or include a limitation to a particular process of producing the intracellular signaling domain, e.g., it does not mean that, to provide the intracellular signaling domain, one must start with a CD3zeta sequence and delete unwanted sequence, or impose mutations, to arrive at the intracellular signaling domain.

The term “signaling domain” refers to the functional portion of a protein which acts by transmitting information within the cell to regulate cellular activity via defined signaling pathways by generating second messengers or functioning as effectors by responding to such messengers.

As used herein, the term “CD19” refers to the Cluster of Differentiation 19 protein, which is an antigenic determinant detectable on leukemia precursor cells. The human and murine amino acid and nucleic acid sequences can be found in a public database, such as GenBank, UniProt and Swiss-Prot. For example, the amino acid sequence of human CD19 can be found as UniProt/Swiss-Prot Accession No. P15391 and the nucleotide sequence encoding of the human CD19 can be found at Accession No. NM_001178098. As used herein, “CD19” includes proteins comprising mutations, e.g., point mutations, fragments, insertions, deletions and splice variants of full length wild-type CD19. CD19 is expressed on most B lineage cancers, including, e.g., acute lymphoblastic leukemia, chronic lymphocyte leukemia and non-Hodgkin lymphoma. Other cells with express CD19 are provided below in the definition of “disease associated with expression of CD19.” It is also an early marker of B cell progenitors. See, e.g., Nicholson et al. Mol. Immun. 34 (16-17): 1157-1165 (1997). In one aspect the antigen-binding portion of the CART recognizes and binds an antigen within the extracellular domain of the CD19 protein. In one aspect, the CD19 protein is expressed on a cancer cell.

The term “antibody,” as used herein, refers to a protein, or polypeptide sequence derived from an immunoglobulin molecule which specifically binds with an antigen. Antibodies can be polyclonal or monoclonal, multiple or single chain, or intact immunoglobulins, and may be derived from natural sources or from recombinant sources. Antibodies can be tetramers of immunoglobulin molecules.

The term “antibody fragment” refers to at least one portion of an antibody, that retains the ability to specifically interact with (e.g., by binding, steric hindrance, stabilizing/destabilizing, spatial distribution) an epitope of an antigen. Examples of antibody fragments include, but are not limited to, Fab, Fab′, F(ab′)2, Fv fragments, scFv antibody fragments, disulfide-linked Fvs (sdFv), a Fd fragment consisting of the VH and CH1 domains, linear antibodies, single domain antibodies such as sdAb (either VL or VH), camelid VHH domains, multi-specific antibodies formed from antibody fragments such as a bivalent fragment comprising two Fab fragments linked by a disulfide bridge at the hinge region, and an isolated CDR or other epitope binding fragments of an antibody. An antigen binding fragment can also be incorporated into single domain antibodies, maxibodies, minibodies, nanobodies, intrabodies, diabodies, triabodies, tetrabodies, v-NAR and bis-scFv (see, e.g., Hollinger and Hudson, Nature Biotechnology 23:1126-1136, 2005). Antigen binding fragments can also be grafted into scaffolds based on polypeptides such as a fibronectin type III (Fn3)(see U.S. Pat. No. 6,703,199, which describes fibronectin polypeptide minibodies).

The term “scFv” refers to a fusion protein comprising at least one antibody fragment comprising a variable region of a light chain and at least one antibody fragment comprising a variable region of a heavy chain, wherein the light and heavy chain variable regions are contiguously linked, e.g., via a synthetic linker, e.g., a short flexible polypeptide linker, and capable of being expressed as a single chain polypeptide, and wherein the scFv retains the specificity of the intact antibody from which it is derived. Unless specified, as used herein an scFv may have the VL and VH variable regions in either order, e.g., with respect to the N-terminal and C-terminal ends of the polypeptide, the scFv may comprise VL-linker-VH or may comprise VH-linker-VL.

The term “complementarity determining region” or “CDR,” as used herein, refers to the sequences of amino acids within antibody variable regions which confer antigen specificity and binding affinity. For example, in general, there are three CDRs in each heavy chain variable region (e.g., HCDR1, HCDR2, and HCDR3) and three CDRs in each light chain variable region (LCDR1, LCDR2, and LCDR3). The precise amino acid sequence boundaries of a given CDR can be determined using any of a number of well-known schemes, including those described by Kabat et al. (1991), “Sequences of Proteins of Immunological Interest,” 5th Ed. Public Health Service, National Institutes of Health, Bethesda, Md. (“Kabat” numbering scheme), Al-Lazikani et al., (1997) JMB 273, 927-948 (“Chothia” numbering scheme), or a combination thereof. Under the Kabat numbering scheme, in some embodiments, the CDR amino acid residues in the heavy chain variable domain (VH) are numbered 31-35 (HCDR1), 50-65 (HCDR2), and 95-102 (HCDR3); and the CDR amino acid residues in the light chain variable domain (VL) are numbered 24-34 (LCDR1), 50-56 (LCDR2), and 89-97 (LCDR3). Under the Chothia numbering scheme, in some embodiments, the CDR amino acids in the VH are numbered 26-32 (HCDR1), 52-56 (HCDR2), and 95-102 (HCDR3); and the CDR amino acid residues in the VL are numbered 26-32 (LCDR1), 50-52 (LCDR2), and 91-96 (LCDR3). In a combined Kabat and Chothia numbering scheme, in some embodiments, the CDRs correspond to the amino acid residues that are part of a Kabat CDR, a Chothia CDR, or both. For instance, in some embodiments, the CDRs correspond to amino acid residues 26-35 (HCDR1), 50-65 (HCDR2), and 95-102 (HCDR3) in a VH, e.g., a mammalian VH, e.g., a human VH; and amino acid residues 24-34 (LCDR1), 50-56 (LCDR2), and 89-97 (LCDR3) in a VL, e.g., a mammalian VL, e.g., a human VL.

As used herein, the term “binding domain” or “antibody molecule” refers to a protein, e.g., an immunoglobulin chain or fragment thereof, comprising at least one immunoglobulin variable domain sequence. The term “binding domain” or “antibody molecule” encompasses antibodies and antibody fragments. In an embodiment, an antibody molecule is a multispecific antibody molecule, e.g., it comprises a plurality of immunoglobulin variable domain sequences, wherein a first immunoglobulin variable domain sequence of the plurality has binding specificity for a first epitope and a second immunoglobulin variable domain sequence of the plurality has binding specificity for a second epitope. In an embodiment, a multispecific antibody molecule is a bispecific antibody molecule. A bispecific antibody has specificity for no more than two antigens. A bispecific antibody molecule is characterized by a first immunoglobulin variable domain sequence which has binding specificity for a first epitope and a second immunoglobulin variable domain sequence that has binding specificity for a second epitope.

The portion of the CAR of the invention comprising an antibody or antibody fragment thereof may exist in a variety of forms where the antigen binding domain is expressed as part of a contiguous polypeptide chain including, for example, a single domain antibody fragment (sdAb), a single chain antibody (scFv), a humanized antibody, or bispecific antibody (Harlow et al., 1999, In: Using Antibodies: A Laboratory Manual, Cold Spring Harbor Laboratory Press, NY; Harlow et al., 1989, In: Antibodies: A Laboratory Manual, Cold Spring Harbor, N.Y.; Houston et al., 1988, Proc. Natl. Acad. Sci. USA 85:5879-5883; Bird et al., 1988, Science 242:423-426). In one aspect, the antigen binding domain of a CAR composition of the invention comprises an antibody fragment. In a further aspect, the CAR comprises an antibody fragment that comprises a scFv.

The term “antibody heavy chain,” refers to the larger of the two types of polypeptide chains present in antibody molecules in their naturally occurring conformations, and which normally determines the class to which the antibody belongs.

The term “antibody light chain,” refers to the smaller of the two types of polypeptide chains present in antibody molecules in their naturally occurring conformations. Kappa (□) and lambda (□) light chains refer to the two major antibody light chain isotypes.

The term “recombinant antibody” refers to an antibody which is generated using recombinant DNA technology, such as, for example, an antibody expressed by a bacteriophage or yeast expression system. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using recombinant DNA or amino acid sequence technology which is available and well known in the art.

The term “antigen” or “Ag” refers to a molecule that provokes an immune response. This immune response may involve either antibody production, or the activation of specific immunologically-competent cells, or both. The skilled artisan will understand that any macromolecule, including virtually all proteins or peptides, can serve as an antigen. Furthermore, antigens can be derived from recombinant or genomic DNA. A skilled artisan will understand that any DNA, which comprises a nucleotide sequences or a partial nucleotide sequence encoding a protein that elicits an immune response therefore encodes an “antigen” as that term is used herein. Furthermore, one skilled in the art will understand that an antigen need not be encoded solely by a full length nucleotide sequence of a gene. It is readily apparent that the present invention includes, but is not limited to, the use of partial nucleotide sequences of more than one gene and that these nucleotide sequences are arranged in various combinations to encode polypeptides that elicit the desired immune response. Moreover, a skilled artisan will understand that an antigen need not be encoded by a “gene” at all. It is readily apparent that an antigen can be generated synthesized or can be derived from a biological sample, or might be macromolecule besides a polypeptide. Such a biological sample can include, but is not limited to a tissue sample, a tumor sample, a cell or a fluid with other biological components.

The terms “compete” or “cross-compete” are used interchangeably herein to refer to the ability of an antibody molecule to interfere with binding of an antibody molecule, e.g., an anti-CD19 or BCMA antibody molecule provided herein, to a target, e.g., human CD19 or BCMA. The interference with binding can be direct or indirect (e.g., through an allosteric modulation of the antibody molecule or the target). The extent to which an antibody molecule is able to interfere with the binding of another antibody molecule to the target, and therefore whether it can be said to compete, can be determined using a competition binding assay, e.g., as described herein. In some embodiments, a competition binding assay is a quantitative competition assay. In some embodiments, a first antibody molecule is said to compete for binding to the target with a second antibody molecule when the binding of the first antibody molecule to the target is reduced by 10% or more, e.g., 20% or more, 30% or more, 40% or more, 50% or more, 55% or more, 60% or more, 65% or more, 70% or more, 75% or more, 80% or more, 85% or more, 90% or more, 95% or more, 98% or more, 99% or more in a competition binding assay (e.g., a competition assay described herein).

As used herein, the term “epitope” refers to the moieties of an antigen (e.g., human CD19 or BCMA) that specifically interact with an antibody molecule. Such moieties, referred to herein as epitopic determinants, typically comprise, or are part of, elements such as amino acid side chains or sugar side chains. An epitopic determinate can be defined, e.g., by methods known in the art or disclosed herein, e.g., by crystallography or by hydrogen-deuterium exchange. At least one or some of the moieties on the antibody molecule, that specifically interact with an epitopic determinant, are typically located in a CDR(s). Typically an epitope has a specific three dimensional structural characteristics. Typically an epitope has specific charge characteristics. Some epitopes are linear epitopes while others are conformational epitopes.

The term “anti-cancer effect” refers to a biological effect which can be manifested by various means, including but not limited to, e.g., a decrease in tumor volume, a decrease in the number of cancer cells, a decrease in the number of metastases, an increase in life expectancy, decrease in cancer cell proliferation, decrease in cancer cell survival, or amelioration of various physiological symptoms associated with the cancerous condition. An “anti-cancer effect” can also be manifested by the ability of the peptides, polynucleotides, cells and antibodies described herein in prevention of the occurrence of cancer in the first place. The term “anti-tumor effect” refers to a biological effect which can be manifested by various means, including but not limited to, e.g., a decrease in tumor volume, a decrease in the number of tumor cells, a decrease in tumor cell proliferation, or a decrease in tumor cell survival.

The term “autologous” refers to any material derived from the same individual to whom it is later to be re-introduced into the individual.

The term “allogeneic” refers to any material derived from a different animal of the same species as the individual to whom the material is introduced. Two or more individuals are said to be allogeneic to one another when the genes at one or more loci are not identical. In some aspects, allogeneic material from individuals of the same species may be sufficiently unlike genetically to interact antigenically.

The term “xenogeneic” refers to a graft derived from an animal of a different species.

The term “cancer” refers to a disease characterized by the uncontrolled growth of aberrant cells. Cancer cells can spread locally or through the bloodstream and lymphatic system to other parts of the body. Examples of various cancers are described herein and include but are not limited to, breast cancer, prostate cancer, ovarian cancer, cervical cancer, skin cancer, pancreatic cancer, colorectal cancer, renal cancer, liver cancer, brain cancer, lymphoma, leukemia, lung cancer and the like. The terms “tumor” and “cancer” are used interchangeably herein, e.g., both terms encompass solid and liquid, e.g., diffuse or circulating, tumors. As used herein, the term “cancer” or “tumor” includes premalignant, as well as malignant cancers and tumors.

The terms “cancer associated antigen” or “tumor antigen” or “proliferative disorder antigen” or “antigen associated with a proliferative disorder” interchangeably refers to a molecule (typically protein, carbohydrate or lipid) that is preferentially expressed on the surface of a cancer cell, either entirely or as a fragment (e.g., MHC/peptide), in comparison to a normal cell, and which is useful for the preferential targeting of a pharmacological agent to the cancer cell. In some embodiments, a tumor antigen is a marker expressed by both normal cells and cancer cells, e.g., a lineage marker, e.g., CD19 on B cells. In certain aspects, the tumor antigens of the present invention are derived from, cancers including but not limited to primary or metastatic melanoma, thymoma, lymphoma, sarcoma, lung cancer, liver cancer, non-Hodgkin lymphoma, Hodgkin lymphoma, leukemias, uterine cancer, cervical cancer, bladder cancer, kidney cancer and adenocarcinomas such as breast cancer, prostate cancer, ovarian cancer, pancreatic cancer, and the like. In some embodiments, the tumor antigen is an antigen that is common to a specific proliferative disorder. In some embodiments, a cancer-associated antigen is a cell surface molecule that is overexpressed in a cancer cell in comparison to a normal cell, for instance, 1-fold over expression, 2-fold overexpression, 3-fold overexpression or more in comparison to a normal cell. In some embodiments, a cancer-associated antigen is a cell surface molecule that is inappropriately synthesized in the cancer cell, for instance, a molecule that contains deletions, additions or mutations in comparison to the molecule expressed on a normal cell. In some embodiments, a cancer-associated antigen will be expressed exclusively on the cell surface of a cancer cell, entirely or as a fragment (e.g., MHC/peptide), and not synthesized or expressed on the surface of a normal cell. In some embodiments, the CARs of the present invention includes CARs comprising an antigen binding domain (e.g., antibody or antibody fragment) that binds to a MHC presented peptide. Normally, peptides derived from endogenous proteins fill the pockets of Major histocompatibility complex (MHC) class I molecules, and are recognized by T cell receptors (TCRs) on CD8+T lymphocytes. The MHC class I complexes are constitutively expressed by all nucleated cells. In cancer, virus-specific and/or tumor-specific peptide/MHC complexes represent a unique class of cell surface targets for immunotherapy. TCR-like antibodies targeting peptides derived from viral or tumor antigens in the context of human leukocyte antigen (HLA)-A1 or HLA-A2 have been described (see, e.g., Sastry et al., J Virol. 2011 85(5):1935-1942; Sergeeva et al., Bood, 2011 117(16):4262-4272; Verma et al., J Immunol 2010 184(4):2156-2165; Willemsen et al., Gene Ther 2001 8(21):1601-1608; Dao et al., Sci Transl Med 2013 5(176):176ra33; Tassev et al., Cancer Gene Ther 2012 19(2):84-100). For example, TCR-like antibody can be identified from screening a library, such as a human scFv phage displayed library.

The phrase “disease associated with expression of CD19” includes, but is not limited to, a disease associated with expression of CD19 (e.g., wild-type or mutant CD19) or condition associated with cells which express, or at any time expressed, CD19 (e.g., wild-type or mutant CD19) including, e.g., proliferative diseases such as a cancer or malignancy or a precancerous condition such as a myelodysplasia, a myelodysplastic syndrome or a preleukemia; or a noncancer related indication associated with cells which express CD19. For the avoidance of doubt, a disease associated with expression of CD19 may include a condition associated with cells which do not presently express CD19, e.g., because CD19 expression has been downregulated, e.g., due to treatment with a molecule targeting CD19, e.g., a CD19 CAR, but which at one time expressed CD19. In one aspect, a cancer associated with expression of CD19 is a hematological cancer. In one aspect, the hematological cancer is a leukemia or a lymphoma. In one aspect, a cancer associated with expression of CD19 includes cancers and malignancies including, but not limited to, e.g., one or more acute leukemias including but not limited to, e.g., B-cell acute Lymphoid Leukemia (BALL), T-cell acute Lymphoid Leukemia (TALL), acute lymphoid leukemia (ALL); one or more chronic leukemias including but not limited to, e.g., chronic myelogenous leukemia (CML), Chronic Lymphoid Leukemia (CLL). Additional cancers or hematologic conditions associated with expression of CD19 comprise, but are not limited to, e.g., B cell prolymphocytic leukemia, blastic plasmacytoid dendritic cell neoplasm, Burkitt's lymphoma, diffuse large B cell lymphoma, Follicular lymphoma, Hairy cell leukemia, small cell- or a large cell-follicular lymphoma, malignant lymphoproliferative conditions, MALT lymphoma, mantle cell lymphoma (MCL), Marginal zone lymphoma, multiple myeloma, myelodysplasia and myelodysplastic syndrome, non-Hodgkin lymphoma, Hodgkin lymphoma, plasmablastic lymphoma, plasmacytoid dendritic cell neoplasm, Waldenstrom macroglobulinemia, and “preleukemia” which are a diverse collection of hematological conditions united by ineffective production (or dysplasia) of myeloid blood cells, and the like. Further diseases associated with expression of CD19 expression include, but not limited to, e.g., atypical and/or non-classical cancers, malignancies, precancerous conditions or proliferative diseases associated with expression of CD19. Non-cancer related indications associated with expression of CD19 include, but are not limited to, e.g., autoimmune disease, (e.g., lupus), inflammatory disorders (allergy and asthma) and transplantation. In some embodiments, the CD19-expressing cells express, or at any time expressed, CD19 mRNA. In an embodiment, the CD19-expressing cells produce a CD19 protein (e.g., wild-type or mutant), and the CD19 protein may be present at normal levels or reduced levels. In an embodiment, the CD19-expressing cells produced detectable levels of a CD19 protein at one point, and subsequently produced substantially no detectable CD19 protein.

The term “conservative sequence modifications” refers to amino acid modifications that do not significantly affect or alter the binding characteristics of the antibody or antibody fragment containing the amino acid sequence. Such conservative modifications include amino acid substitutions, additions and deletions. Modifications can be introduced into an antibody or antibody fragment of the invention by standard techniques known in the art, such as site-directed mutagenesis and PCR-mediated mutagenesis. Conservative amino acid substitutions are ones in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, one or more amino acid residues within a CAR of the invention can be replaced with other amino acid residues from the same side chain family and the altered CAR can be tested using the functional assays described herein.

The term “stimulation,” refers to a primary response induced by binding of a stimulatory molecule (e.g., a TCR/CD3 complex or CAR) with its cognate ligand (or tumor antigen in the case of a CAR) thereby mediating a signal transduction event, such as, but not limited to, signal transduction via the TCR/CD3 complex or signal transduction via the appropriate NK receptor or signaling domains of the CAR. Stimulation can mediate altered expression of certain molecules.

The term “stimulatory molecule,” refers to a molecule expressed by an immune cell, e.g., T cell, NK cell, or B cell) that provides the cytoplasmic signaling sequence(s) that regulate activation of the immune cell in a stimulatory way for at least some aspect of the immune cell signaling pathway. In one aspect, the signal is a primary signal that is initiated by, for instance, binding of a TCR/CD3 complex with an MHC molecule loaded with peptide, and which leads to mediation of a T cell response, including, but not limited to, proliferation, activation, differentiation, and the like. A primary cytoplasmic signaling sequence (also referred to as a “primary signaling domain”) that acts in a stimulatory manner may contain a signaling motif which is known as immunoreceptor tyrosine-based activation motif or ITAM. Examples of an ITAM containing cytoplasmic signaling sequence that is of particular use in the invention includes, but is not limited to, those derived from CD3 zeta, common FcR gamma (FCER1G), Fc gamma RIIa, FcR beta (Fc Epsilon Rib), CD3 gamma, CD3 delta, CD3 epsilon, CD79a, CD79b, DAP10, and DAP12. In a specific CAR of the invention, the intracellular signaling domain in any one or more CARS of the invention comprises an intracellular signaling sequence, e.g., a primary signaling sequence of CD3-zeta. In a specific CAR of the invention, the primary signaling sequence of CD3-zeta is the sequence provided as SEQ ID NO:17, or the equivalent residues from a non-human species, e.g., mouse, rodent, monkey, ape and the like. In a specific CAR of the invention, the primary signaling sequence of CD3-zeta is the sequence as provided in SEQ ID NO:43, or the equivalent residues from a non-human species, e.g., mouse, rodent, monkey, ape and the like.

The term “antigen presenting cell” or “APC” refers to an immune system cell such as an accessory cell (e.g., a B-cell, a dendritic cell, and the like) that displays a foreign antigen complexed with major histocompatibility complexes (MHC's) on its surface. T-cells may recognize these complexes using their T-cell receptors (TCRs). APCs process antigens and present them to T-cells.

“Immune effector cell,” as that term is used herein, refers to a cell that is involved in an immune response, e.g., in the promotion of an immune effector response. Examples of immune effector cells include T cells, e.g., alpha/beta T cells and gamma/delta T cells, B cells, natural killer (NK) cells, natural killer T (NK-T) cells, mast cells, and myeloid-derived phagocytes.

“Immune effector function or immune effector response,” as that term is used herein, refers to function or response, e.g., of an immune effector cell, that enhances or promotes an immune attack of a target cell. E.g., an immune effector function or response refers a property of a T or NK cell that promotes killing or the inhibition of growth or proliferation, of a target cell. In the case of a T cell, primary stimulation and co-stimulation are examples of immune effector function or response.

The term “effector function” refers to a specialized function of a cell. Effector function of a T cell, for example, may be cytolytic activity or helper activity including the secretion of cytokines.

An “intracellular signaling domain,” as the term is used herein, refers to an intracellular portion of a molecule. The intracellular signaling domain can generate a signal that promotes an immune effector function of the CAR containing cell, e.g., a CART cell. Examples of immune effector function, e.g., in a CART cell, include cytolytic activity and helper activity, including the secretion of cytokines. In embodiments, the intracellular signal domain is the portion of the protein which transduces the effector function signal and directs the cell to perform a specialized function. While the entire intracellular signaling domain can be employed, in many cases it is not necessary to use the entire chain. To the extent that a truncated portion of the intracellular signaling domain is used, such truncated portion may be used in place of the intact chain as long as it transduces the effector function signal. The term intracellular signaling domain is thus meant to include any truncated portion of the intracellular signaling domain sufficient to transduce the effector function signal.

In an embodiment, the intracellular signaling domain can comprise a primary intracellular signaling domain. Exemplary primary intracellular signaling domains include those derived from the molecules responsible for primary stimulation, or antigen dependent simulation. In an embodiment, the intracellular signaling domain can comprise a costimulatory intracellular domain. Exemplary costimulatory intracellular signaling domains include those derived from molecules responsible for costimulatory signals, or antigen independent stimulation. For example, in the case of a CART, a primary intracellular signaling domain can comprise a cytoplasmic sequence of a T cell receptor, and a costimulatory intracellular signaling domain can comprise cytoplasmic sequence from co-receptor or costimulatory molecule.

A primary intracellular signaling domain can comprise a signaling motif which is known as an immunoreceptor tyrosine-based activation motif or ITAM. Examples of ITAM containing primary cytoplasmic signaling sequences include, but are not limited to, those derived from CD3 zeta, FcR gamma, common FcR gamma (FCER1G), Fc gamma RIIa, FcR beta (Fc Epsilon Rib), CD3 gamma, CD3 delta, CD3 epsilon, CD22, CD79a, CD79b, CD278 (“ICOS”), FcεRI, CD66d, CD32, DAP10 and DAP12.

The term “zeta” or alternatively “zeta chain”, “CD3-zeta” or “TCR-zeta” is defined as the protein provided as GenBank Acc. No. BAG36664.1, or the equivalent residues from a non-human species, e.g., mouse, rodent, monkey, ape and the like, and a “zeta stimulatory domain” or alternatively a “CD3-zeta stimulatory domain” or a “TCR-zeta stimulatory domain” is defined as the amino acid residues from the cytoplasmic domain of the zeta chain, or functional derivatives thereof, that are sufficient to functionally transmit an initial signal necessary for T cell activation. In one aspect the cytoplasmic domain of zeta comprises residues 52 through 164 of GenBank Acc. No. BAG36664.1 or the equivalent residues from a non-human species, e.g., mouse, rodent, monkey, ape and the like, that are functional orthologs thereof. In one aspect, the “zeta stimulatory domain” or a “CD3-zeta stimulatory domain” is the sequence provided as SEQ ID NO:17. In one aspect, the “zeta stimulatory domain” or a “CD3-zeta stimulatory domain” is the sequence provided as SEQ ID NO:43.

The term “costimulatory molecule” refers to the cognate binding partner on a T cell that specifically binds with a costimulatory ligand, thereby mediating a costimulatory response by the T cell, such as, but not limited to, proliferation. Costimulatory molecules are cell surface molecules other than antigen receptors or their ligands that contribute to an efficient immune response. Costimulatory molecules include, but are not limited to an MHC class I molecule, TNF receptor proteins, Immunoglobulin-like proteins, cytokine receptors, integrins, signalling lymphocytic activation molecules (SLAM proteins), activating NK cell receptors, BTLA, a Toll ligand receptor, OX40, CD2, CD7, CD27, CD28, CD30, CD40, CDS, ICAM-1, LFA-1 (CD11a/CD18), 4-1BB (CD137), B7-H3, CDS, ICAM-1, ICOS (CD278), GITR, BAFFR, LIGHT, HVEM (LIGHTR), KIRDS2, SLAMF7, NKp80 (KLRF1), NKp44, NKp30, NKp46, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, NKG2D, NKG2C, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, and a ligand that specifically binds with CD83.

A costimulatory intracellular signaling domain refers to the intracellular portion of a costimulatory molecule. The intracellular signaling domain can comprise the entire intracellular portion, or the entire native intracellular signaling domain, of the molecule from which it is derived, or a functional fragment or derivative thereof.

The term “4-1BB” refers to a member of the TNFR superfamily with an amino acid sequence provided as GenBank Acc. No. AAA62478.2, or the equivalent residues from a non-human species, e.g., mouse, rodent, monkey, ape and the like; and a “4-1BB costimulatory domain” is defined as amino acid residues 214-255 of GenBank Acc. No. AAA62478.2, or the equivalent residues from a non-human species, e.g., mouse, rodent, monkey, ape and the like. In one aspect, the “4-1BB costimulatory domain” is the sequence provided as SEQ ID NO:16 or the equivalent residues from a non-human species, e.g., mouse, rodent, monkey, ape and the like.

The term “encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (e.g., rRNA, tRNA and mRNA) or a defined sequence of amino acids and the biological properties resulting therefrom. Thus, a gene, cDNA, or RNA, encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or a RNA may also include introns to the extent that the nucleotide sequence encoding the protein may in some version contain an intron(s).

The term “effective amount” or “therapeutically effective amount” are used interchangeably herein, and refer to an amount of a compound, formulation, material, or composition, as described herein effective to achieve a particular biological result.

The term “endogenous” refers to any material from or produced inside an organism, cell, tissue or system.

The term “exogenous” refers to any material introduced from or produced outside an organism, cell, tissue or system.

The term “expression” refers to the transcription and/or translation of a particular nucleotide sequence driven by a promoter.

The term “transfer vector” refers to a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “transfer vector” includes an autonomously replicating plasmid or a virus. The term should also be construed to further include non-plasmid and non-viral compounds which facilitate transfer of nucleic acid into cells, such as, for example, a polylysine compound, liposome, and the like. Examples of viral transfer vectors include, but are not limited to, adenoviral vectors, adeno-associated virus vectors, retroviral vectors, lentiviral vectors, and the like.

The term “expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, including cosmids, plasmids (e.g., naked or contained in liposomes) and viruses (e.g., lentiviruses, retroviruses, adenoviruses, and adeno-associated viruses) that incorporate the recombinant polynucleotide.

The term “lentivirus” refers to a genus of the Retroviridae family. Lentiviruses are unique among the retroviruses in being able to infect non-dividing cells; they can deliver a significant amount of genetic information into the DNA of the host cell, so they are one of the most efficient methods of a gene delivery vector. HIV, SIV, and FIV are all examples of lentiviruses.

The term “lentiviral vector” refers to a vector derived from at least a portion of a lentivirus genome, including especially a self-inactivating lentiviral vector as provided in Milone et al., Mol. Ther. 17(8): 1453-1464 (2009). Other examples of lentivirus vectors that may be used in the clinic, include but are not limited to, e.g., the LENTIVECTOR® gene delivery technology from Oxford BioMedica, the LENTIMAX™ vector system from Lentigen and the like. Nonclinical types of lentiviral vectors are also available and would be known to one skilled in the art.

The term “homologous” or “identity” refers to the subunit sequence identity between two polymeric molecules, e.g., between two nucleic acid molecules, such as, two DNA molecules or two RNA molecules, or between two polypeptide molecules. When a subunit position in both of the two molecules is occupied by the same monomeric subunit; e.g., if a position in each of two DNA molecules is occupied by adenine, then they are homologous or identical at that position. The homology between two sequences is a direct function of the number of matching or homologous positions; e.g., if half (e.g., five positions in a polymer ten subunits in length) of the positions in two sequences are homologous, the two sequences are 50% homologous; if 90% of the positions (e.g., 9 of 10), are matched or homologous, the two sequences are 90% homologous.

“Humanized” forms of non-human (e.g., murine) antibodies are chimeric immunoglobulins, immunoglobulin chains or fragments thereof (such as Fv, Fab, Fab′, F(ab′)2 or other antigen-binding subsequences of antibodies) which contain minimal sequence derived from non-human immunoglobulin. For the most part, humanized antibodies and antibody fragments thereof are human immunoglobulins (recipient antibody or antibody fragment) in which residues from a complementary-determining region (CDR) of the recipient are replaced by residues from a CDR of a non-human species (donor antibody) such as mouse, rat or rabbit having the desired specificity, affinity, and capacity. In some instances, Fv framework region (FR) residues of the human immunoglobulin are replaced by corresponding non-human residues. Furthermore, a humanized antibody/antibody fragment can comprise residues which are found neither in the recipient antibody nor in the imported CDR or framework sequences. These modifications can further refine and optimize antibody or antibody fragment performance. In general, the humanized antibody or antibody fragment thereof will comprise substantially all of at least one, and typically two, variable domains, in which all or substantially all of the CDR regions correspond to those of a non-human immunoglobulin and all or a significant portion of the FR regions are those of a human immunoglobulin sequence. The humanized antibody or antibody fragment can also comprise at least a portion of an immunoglobulin constant region (Fc), typically that of a human immunoglobulin. For further details, see Jones et al., Nature, 321: 522-525, 1986; Reichmann et al., Nature, 332: 323-329, 1988; Presta, Curr. Op. Struct. Biol., 2: 593-596, 1992.

“Fully human” refers to an immunoglobulin, such as an antibody or antibody fragment, where the whole molecule is of human origin or consists of an amino acid sequence identical to a human form of the antibody or immunoglobulin.

The term “isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

In the context of the present invention, the following abbreviations for the commonly occurring nucleic acid bases are used. “A” refers to adenosine, “C” refers to cytosine, “G” refers to guanosine, “T” refers to thymidine, and “U” refers to uridine.

The term “operably linked” or “transcriptional control” refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Operably linked DNA sequences can be contiguous with each other and, e.g., where necessary to join two protein coding regions, are in the same reading frame.

The term “parenteral” administration of an immunogenic composition includes, e.g., subcutaneous (s.c.), intravenous (i.v.), intramuscular (i.m.), or intrasternal injection, intratumoral, or infusion techniques.

The term “nucleic acid” or “polynucleotide” refers to deoxyribonucleic acids (DNA) or ribonucleic acids (RNA) and polymers thereof in either single- or double-stranded form. The term “nucleic acid” includes a gene, cDNA, or an mRNA. In one embodiment, the nucleic acid molecule is synthetic (e.g., chemically synthesized) or recombinant. Unless specifically limited, the term encompasses nucleic acids containing analogues or derivatives of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)).

The terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. A polypeptide includes a natural peptide, a recombinant peptide, or a combination thereof.

As used herein, the term “plurality” refers to two or more.

The term “promoter” refers to a DNA sequence recognized by the synthetic machinery of the cell, or introduced synthetic machinery, required to initiate the specific transcription of a polynucleotide sequence.

The term “promoter/regulatory sequence” refers to a nucleic acid sequence which is required for expression of a gene product operably linked to the promoter/regulatory sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

The term “constitutive” promoter refers to a nucleotide sequence which, when operably linked with a polynucleotide which encodes or specifies a gene product, causes the gene product to be produced in a cell under most or all physiological conditions of the cell.

The term “inducible” promoter refers to a nucleotide sequence which, when operably linked with a polynucleotide which encodes or specifies a gene product, causes the gene product to be produced in a cell substantially only when an inducer which corresponds to the promoter is present in the cell.

The term “tissue-specific” promoter refers to a nucleotide sequence which, when operably linked with a polynucleotide encodes or specified by a gene, causes the gene product to be produced in a cell substantially only if the cell is a cell of the tissue type corresponding to the promoter.

The term “flexible polypeptide linker” or “linker” as used in the context of a scFv refers to a peptide linker that consists of amino acids such as glycine and/or serine residues used alone or in combination, to link variable heavy and variable light chain regions together. In one embodiment, the flexible polypeptide linker is a Gly/Ser linker and comprises the amino acid sequence (Gly-Gly-Gly-Ser)n, where n is a positive integer equal to or greater than 1. For example, n=1, n=2, n=3. n=4, n=5, n=6, n=7, n=8, n=9 and n=10 (SEQ ID NO:105). In one embodiment, the flexible polypeptide linkers include, but are not limited to, (Gly4 Ser)4 (SEQ ID NO:106) or (Gly4 Ser)3 (SEQ ID NO:107). In another embodiment, the linkers include multiple repeats of (Gly2Ser), (GlySer) or (Gly3Ser) (SEQ ID NO:108). Also included within the scope of the invention are linkers described in WO2012/138475, incorporated herein by reference.

As used herein, a 5′ cap (also termed an RNA cap, an RNA 7-methylguanosine cap or an RNA m⁷G cap) is a modified guanine nucleotide that has been added to the “front” or 5′ end of a eukaryotic messenger RNA shortly after the start of transcription. The 5′ cap consists of a terminal group which is linked to the first transcribed nucleotide. Its presence is important for recognition by the ribosome and protection from RNases. Cap addition is coupled to transcription, and occurs co-transcriptionally, such that each influences the other. Shortly after the start of transcription, the 5′ end of the mRNA being synthesized is bound by a cap-synthesizing complex associated with RNA polymerase. This enzymatic complex catalyzes the chemical reactions that are required for mRNA capping. Synthesis proceeds as a multi-step biochemical reaction. The capping moiety can be modified to modulate functionality of mRNA such as its stability or efficiency of translation.

As used herein, “in vitro transcribed RNA” refers to RNA, e.g., mRNA, that has been synthesized in vitro. Generally, the in vitro transcribed RNA is generated from an in vitro transcription vector. The in vitro transcription vector comprises a template that is used to generate the in vitro transcribed RNA.

As used herein, a “poly(A)” is a series of adenosines attached by polyadenylation to the mRNA. In some embodiments of a construct for transient expression, the polyA is between 50 and 5000 (SEQ ID NO: 28), e.g., greater than 64, e.g., greater than 100, e.g., than 300 or 400. Poly(A) sequences can be modified chemically or enzymatically to modulate mRNA functionality such as localization, stability or efficiency of translation.

As used herein, “polyadenylation” refers to the covalent linkage of a polyadenylyl moiety, or its modified variant, to a messenger RNA molecule. In eukaryotic organisms, most messenger RNA (mRNA) molecules are polyadenylated at the 3′ end. The 3′ poly(A) tail is a long sequence of adenine nucleotides (often several hundred) added to the pre-mRNA through the action of an enzyme, polyadenylate polymerase. In higher eukaryotes, the poly(A) tail is added onto transcripts that contain a specific sequence, the polyadenylation signal. The poly(A) tail and the protein bound to it aid in protecting mRNA from degradation by exonucleases. Polyadenylation is also important for transcription termination, export of the mRNA from the nucleus, and translation. Polyadenylation occurs in the nucleus immediately after transcription of DNA into RNA, but additionally can also occur later in the cytoplasm. After transcription has been terminated, the mRNA chain is cleaved through the action of an endonuclease complex associated with RNA polymerase. The cleavage site is usually characterized by the presence of the base sequence AAUAAA near the cleavage site. After the mRNA has been cleaved, adenosine residues are added to the free 3′ end at the cleavage site.

As used herein, “transient” refers to expression of a non-integrated transgene for a period of hours, days or weeks, wherein the period of time of expression is less than the period of time for expression of the gene if integrated into the genome or contained within a stable plasmid replicon in the host cell.

The term “signal transduction pathway” refers to the biochemical relationship between a variety of signal transduction molecules that play a role in the transmission of a signal from one portion of a cell to another portion of a cell. The phrase “cell surface receptor” includes molecules and complexes of molecules capable of receiving a signal and transmitting signal across the membrane of a cell.

The term “subject” is intended to include living organisms in which an immune response can be elicited (e.g., mammals, human).

The term, a “substantially purified” cell refers to a cell that is essentially free of other cell types. A substantially purified cell also refers to a cell which has been separated from other cell types with which it is normally associated in its naturally occurring state. In some instances, a population of substantially purified cells refers to a homogenous population of cells. In other instances, this term refers simply to cell that have been separated from the cells with which they are naturally associated in their natural state. In some aspects, the cells are cultured in vitro. In other aspects, the cells are not cultured in vitro.

The term “therapeutic” as used herein means a treatment. A therapeutic effect is obtained by reduction, suppression, remission, or eradication of a disease state.

The term “prophylaxis” as used herein means the prevention of or protective treatment for a disease or disease state.

In the context of the present invention, “tumor antigen” or “hyperproliferative disorder antigen” or “antigen associated with a hyperproliferative disorder” refers to antigens that are common to specific hyperproliferative disorders. In certain aspects, the hyperproliferative disorder antigens of the present invention are derived from, cancers including but not limited to primary or metastatic melanoma, thymoma, lymphoma, sarcoma, lung cancer, liver cancer, non-Hodgkin lymphoma, Hodgkin lymphoma, leukemias, uterine cancer, cervical cancer, bladder cancer, kidney cancer and adenocarcinomas such as breast cancer, prostate cancer, ovarian cancer, pancreatic cancer, and the like.

The term “transfected” or “transformed” or “transduced” refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

A subject “responds” to treatment if a parameter of a cancer (e.g., a hematological cancer, e.g., cancer cell growth, proliferation and/or survival) in the subject is retarded or reduced by a detectable amount, e.g., about 5%, 10%, 20%, 30%, 40%, 50%, 60%, 70%, 80%, 90% or more as determined by any appropriate measure, e.g., by mass, cell count or volume. In one example, a subject responds to treatment if the subject experiences a life expectancy extended by about 5%, 10%, 20%, 30%, 40%, 50% or more beyond the life expectancy predicted if no treatment is administered. In another example, a subject responds to treatment, if the subject has an increased disease-free survival, overall survival or increased time to progression. Several methods can be used to determine if a patient responds to a treatment including, for example, criteria provided by NCCN Clinical Practice Guidelines in Oncology (NCCN Guidelines®). For example, in the context of B-ALL, a complete response or complete responder, may involve one or more of: <5% BM blast, >1000 neutrophil/ANC (/μL). >100,000 platelets (/μL) with no circulating blasts or extramedullary disease (no lymphadenopathy, splenomegaly, skin/gum infiltration/testicular mass/CNS involvement), Trilineage hematopoiesis, and no recurrence for 4 weeks. A partial responder may involve one or more of >50% reduction in BM blast, >1000 neutrophil/ANC (/μL). >100,000 platelets (/μL). A non-responder can show disease progression, e.g., >25% in BM blasts.

“Refractory” as used herein refers to a disease, e.g., cancer, that does not respond to a treatment. In embodiments, a refractory cancer can be resistant to a treatment before or at the beginning of the treatment. In other embodiments, the refractory cancer can become resistant during a treatment. A refractory cancer is also called a resistant cancer.

The term “relapse” as used herein refers to reappearance of a cancer after an initial period of responsiveness (e.g., complete response or partial response). The initial period of responsiveness may involve the level of cancer cells falling below a certain threshold, e.g., below 20%, 1%, 10%, 5%, 4%, 3%, 2%, or 1%. The reappearance may involve the level of cancer cells rising above a certain threshold, e.g., above 20%, 1%, 10%, 5%, 4%, 3%, 2%, or 1%. For example, e.g., in the context of B-ALL, the reappearance may involve, e.g., a reappearance of blasts in the blood, bone marrow (>5%), or any extramedullary site, after a complete response. A complete response, in this context, may involve <5% BM blast. More generally, in an embodiment, a response (e.g., complete response or partial response) can involve the absence of detectable MRD (minimal residual disease). In an embodiment, the initial period of responsiveness lasts at least 1, 2, 3, 4, 5, or 6 days; at least 1, 2, 3, or 4 weeks; at least 1, 2, 3, 4, 6, 8, 10, or 12 months; or at least 1, 2, 3, 4, or 5 years.

In some embodiments, a therapy that includes a CD19 inhibitor, e.g., a CD19 CAR therapy, may relapse or be refractory to treatment. The relapse or resistance can be caused by CD19 loss (e.g., an antigen loss mutation) or other CD19 alteration that reduces the level of CD19 (e.g., caused by clonal selection of CD19-negative clones). A cancer that harbors such CD19 loss or alteration is referred to herein as a “CD19-negative cancer” or a “CD19-negative relapsed cancer”). It shall be understood that a CD19-negative cancer need not have 100% loss of CD19, but a sufficient reduction to reduce the effectiveness of a CD19 therapy such that the cancer relapses or becomes refractory. In some embodiments, a CD19-negative cancer results from a CD19 CAR therapy.

The term “specifically binds,” refers to an antibody, or a ligand, which recognizes and binds with a binding partner (e.g., a stimulatory tumor antigen) protein present in a sample, but which antibody or ligand does not substantially recognize or bind other molecules in the sample.

As used herein, the term “pharmaceutically acceptable salt” refers to those salts which are, within the scope of sound medical judgment, suitable for use in contact with the tissues of subjects without undue toxicity, irritation, allergic response and the like, and are commensurate with a reasonable benefit/risk ratio. Pharmaceutically acceptable salts are well known in the art. For example, Berge et al. describes pharmaceutically acceptable salts in detail in J. Pharmaceutical Sciences (1977) 66:1-19.

“Regulatable chimeric antigen receptor (RCAR),” as that term is used herein, refers to a set of polypeptides, typically two in the simplest embodiments, which when in a RCARX cell, provides the RCARX cell with specificity for a target cell, typically a cancer cell, and with regulatable intracellular signal generation or proliferation, which can optimize an immune effector property of the RCARX cell. An RCARX cell relies at least in part, on an antigen binding domain to provide specificity to a target cell that comprises the antigen bound by the antigen binding domain. In an embodiment, an RCAR includes a dimerization switch that, upon the presence of a dimerization molecule, can couple an intracellular signaling domain to the antigen binding domain.

“Membrane anchor” or “membrane tethering domain”, as that term is used herein, refers to a polypeptide or moiety, e.g., a myristoyl group, sufficient to anchor an extracellular or intracellular domain to the plasma membrane.

“Switch domain,” as that term is used herein, e.g., when referring to an RCAR, refers to an entity, typically a polypeptide-based entity, that, in the presence of a dimerization molecule, associates with another switch domain. The association results in a functional coupling of a first entity linked to, e.g., fused to, a first switch domain, and a second entity linked to, e.g., fused to, a second switch domain. A first and second switch domain are collectively referred to as a dimerization switch. In embodiments, the first and second switch domains are the same as one another, e.g., they are polypeptides having the same primary amino acid sequence, and are referred to collectively as a homodimerization switch. In embodiments, the first and second switch domains are different from one another, e.g., they are polypeptides having different primary amino acid sequences, and are referred to collectively as a heterodimerization switch. In embodiments, the switch is intracellular. In embodiments, the switch is extracellular. In embodiments, the switch domain is a polypeptide-based entity, e.g., FKBP or FRB-based, and the dimerization molecule is small molecule, e.g., a rapalogue. In embodiments, the switch domain is a polypeptide-based entity, e.g., an scFv that binds a myc peptide, and the dimerization molecule is a polypeptide, a fragment thereof, or a multimer of a polypeptide, e.g., a myc ligand or multimers of a myc ligand that bind to one or more myc scFvs. In embodiments, the switch domain is a polypeptide-based entity, e.g., myc receptor, and the dimerization molecule is an antibody or fragments thereof, e.g., myc antibody.

“Dimerization molecule,” as that term is used herein, e.g., when referring to an RCAR, refers to a molecule that promotes the association of a first switch domain with a second switch domain. In embodiments, the dimerization molecule does not naturally occur in the subject, or does not occur in concentrations that would result in significant dimerization. In embodiments, the dimerization molecule is a small molecule, e.g., rapamycin or a rapalogue, e.g., RAD001.

The term “low, immune enhancing, dose” when used in conjunction with an mTOR inhibitor, e.g., an allosteric mTOR inhibitor, e.g., RAD001 or rapamycin, or a catalytic mTOR inhibitor, refers to a dose of mTOR inhibitor that partially, but not fully, inhibits mTOR activity, e.g., as measured by the inhibition of P70 S6 kinase activity. Methods for evaluating mTOR activity, e.g., by inhibition of P70 S6 kinase, are discussed herein. The dose is insufficient to result in complete immune suppression but is sufficient to enhance the immune response. In an embodiment, the low, immune enhancing, dose of mTOR inhibitor results in a decrease in the number of PD-1 positive T cells and/or an increase in the number of PD-1 negative T cells, or an increase in the ratio of PD-1 negative T cells/PD-1 positive T cells. In an embodiment, the low, immune enhancing, dose of mTOR inhibitor results in an increase in the number of naive T cells. In an embodiment, the low, immune enhancing, dose of mTOR inhibitor results in one or more of the following:
an increase in the expression of one or more of the following markers: CD62L^{high}, CD127^{high}, CD27⁺, and BCL2, e.g., on memory T cells, e.g., memory T cell precursors;
a decrease in the expression of KLRG1, e.g., on memory T cells, e.g., memory T cell precursors; and
an increase in the number of memory T cell precursors, e.g., cells with any one or combination of the following characteristics: increased CD62L^{high}, increased CD127^{high}, increased CD27⁺, decreased KLRG1, and increased BCL2;
   wherein any of the changes described above occurs, e.g., at least transiently, e.g., as compared to a non-treated subject.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. As another example, a range such as 95-99% identity, includes something with 95%, 96%, 97%, 98% or 99% identity, and includes subranges such as 96-99%, 96-98%, 96-97%, 97-99%, 97-98% and 98-99% identity. This applies regardless of the breadth of the range.

### DESCRIPTION

Chimeric antigen receptor-engineered T-cell (CAR T) therapy has shown promise in the treatment of certain cancers, e.g., hematological cancers. CAR-T therapy can be optimized using approaches that include alterations to dosing, e.g., therapeutic, regimens, e.g., as described herein. The present disclosure provides, inter alia, dosing regimens that can, e.g., improve the efficacy or CAR-T therapy and/or reduce side effects associated with CAR-T therapy.

Provided herein, inter alia, is a method of treating a subject with a CAR-expressing cell therapy, e.g., a CD19 CAR expressing cell therapy, comprising administering to the subject a CAR-expressing cell therapy, e.g., a CAR19 expressing cell therapy, wherein the CAR-expressing cell therapy is administered less than 30 days, e.g., less than 29, 28, 27, 26, 25, 24, 23, 22, 21, 20, 19, 18, 17, 16, 15, 14, 13, 12, 11, 10, 9, 8, 7, 6, 5, 4, 3, 2, or 1 days, after administration of a lymphodepleting therapy comprising radiotherapy. Also provided herein, is a method of treating, e.g., preventing, cytokine release syndrome (CRS) with a CAR-expressing cell therapy, e.g., a CAR19 expressing cell therapy, in a subject in need thereof, comprising administering to the subject a lymphodepleting therapy comprising radiotherapy, thereby preventing CRS in the subject. Furthermore, the disclosure provides a method of treating a subject comprising administering to the subject a CAR-expressing cell therapy, e.g., a CAR19 expressing cell therapy, wherein the CAR-expressing cell therapy is administered after stem cell therapy (SCT), e.g., autologous SCT or allogeneic SCT, wherein the subject has not responded, e.g., relapsed, to the SCT, thereby treating the subject. In some embodiments, wherein the CAR-expressing cell therapy is administered after relapse from SCT therapy, e.g., about 1-6 months (e.g., about 1.1-1.5, 1.5-2.0, 2.0-2.5, 2.5-3, 3-3.5, 3.5-4, 4-4.5. 4.5-5, 5-5.5, or 5.5-6 months) after relapse. Additional features of the methods of treatment disclosed herein are described in further detail below. Also disclosed herein are CAR-expressing cell therapies, e.g., CAR19 expressing cell therapy, and methods of making and using the same.

### Stem Cell Therapy

Stem cell therapy as used herein is also referred to as stem cell transplantation. In one aspect, the disclosure provides a method of treating a subject with a CAR-expressing cell therapy in combination with stem cell therapy (SCT), e.g., autologous SCT or allogeneic SCT. In some embodiments, the SCT is administered prior to administration of the CAR-expressing cell therapy. In some embodiments, the CAR-expressing cell therapy is administered, after relapse from SCT, e.g., about 1-12 months, e.g., about 1-3, 3-6, 6-9, or 9-12 months, after relapse. In some embodiments, the SCT is administered, after relapse from SCT, e.g., about 1-6 months (e.g., about 1.1-1.5, 1.5-2.0, 2.0-2.5, 2.5-3, 3-3.5, 3.5-4, 4-4.5. 4.5-5, 5-5.5, or 5.5-6 months) after relapse. In some embodiments, the subject is a pediatric subject, e.g., as described herein, and the subject has a hematological cancer, e.g., ALL, e.g., B-ALL, e.g., relapsed and/or refractory B-ALL. In some embodiments, the subject is an adolescent, e.g., as described herein, and the subject has a hematolological cancer, e.g., ALL, e.g., B-ALL, e.g., relapsed and/or refractory B-ALL. In some embodiments, the subject is a young adult, e.g., as described herein and the subject has ALL, e.g., B-ALL. In some embodiments, the subject has NHL, e.g., relapsed and/or refractory NHL.

In some embodiments, SCT comprises administration of cells, e.g., hematopoietic cells, e.g., hematopoietic stem cells. In some embodiments, the cells, e.g., hematopoietic cells, e.g., HSCs, are derived, e.g., obtained, from bone marrow, cord blood, or peripheral blood.

### Radiotherapy

Radiotherapy as used herein is also referred to a radiation therapy (RT). In some embodiments, radiotherapy is used as a lymphodepleting therapy. In some embodiments, the dose of radiotherapy used is a low dose radiotherapy. In some embodiments, the dose of radiotherapy used is a high dose radiotherapy.

In one aspect, the disclosure provides a method of treating a subject with a CAR-expressing cell therapy in combination with radiotherapy. In some embodiments, the CAR-expressing cell therapy is administered after the administration of radiotherapy. In some embodiments, the CAR-expressing cell therapy is administered less than 30 days, e.g., less than 29, 28, 27, 26, 25, 24, 23, 22, 21, 20, 19, 18, 17, 16, 15, 14, 13, 12, 11, 10, 9, 8, 7, 6, 5, 4, 3, 2, or 1 days, after administration of a lymphodepleting therapy comprising radiotherapy.

In another aspect, the disclosure provides a method of treating, e.g., preventing CRS in a subject with a CAR-expressing cell therapy, e.g., a CAR19 expressing cell therapy, comprising administering to the subject a lymphodepleting therapy comprising radiotherapy. In some embodiments, the radiotherapy is administered less than 30 days, e.g., less than 29, 28, 27, 26, 25, 24, 23, 22, 21, 20, 19, 18, 17, 16, 15, 14, 13, 12, 11, 10, 9, 8, 7, 6, 5, 4, 3, 2, or 1 days, prior to the administration of the CAR-expressing cell therapy. In some embodiments, the subject (i) is at risk of developing, has, or is diagnosed with CRS; (ii) is identified or has previously been identified as being at risk for CRS; and/or (iii) has been, is being, or will be administered a CAR therapy, e.g., a CD19 CAR-expressing cell. In some embodiments, the CRS is a severe CRS, e.g., grade 4 or 5 CRS, or less than severe CRS, e.g., grade 1, 2, or 3 CRS.

In some embodiments, the radiotherapy is administered at a dose of about 1-100 Gy, e.g., about 1-10, 10-20, 20-30, 30-40, 40-50, 50-60, 60-70, 80-90, or 90-100 Gy, or about 1-99, 5-90, 10-85, 15-80, 20-75, 25-70, 30-65, 35-60, 40-55 or 45-50 Gy. In some embodiments, the radiotherapy is administered at a dose of about 50 Gy, e.g., about 49, 48, 47, 46, 45, 44, 43, 42, 41, 40, 35, 30, 25, 24, 23, 22, 21, 20, 15, 10, 9, 8, 7, 6, 5, 4, 3, 2.9, 2.8, 2.7, 2.6, 2.5, 2.4, 2.3, 2.2, 2.1, 2 or 1 Gy. In some embodiments, the radiotherapy is administered at a dose of about 40 Gy, e.g., 40 Gy. In some embodiments, the radiotherapy is administered at a dose of about 22 Gy, e.g., 22 Gy. In some embodiments, the radiotherapy is administered at a dose of about 4 Gy, e.g., 4 Gy. In some embodiments, the radiotherapy is administered at a dose of about 2.2 Gy, e.g., 2.2 Gy.

In some embodiments of any of the methods disclosed herein, the radiotherapy is administered as a single dose, e.g., at a dose described herein.

In some embodiments of any of the methods disclosed herein, the radiotherapy is administered as a fractionated dose, e.g., one or more doses (e.g., two, three or four partial doses). In some embodiments, the radiotherapy consists of a total dose administered as a fractionated dose, e.g., one or more doses (e.g., two, three or four partial doses). In some embodiments, the radiotherapy is administered as two doses, e.g., two fractionated doses, e.g., a first dose and a second dose. In some embodiments, the first dose, e.g., first fractionated dose, is at least about 40 fold higher, e.g., about 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, or 1.5 fold higher than the subsequent dose, e.g., second dose, e.g., second fractionated dose. In some embodiments, the first dose, e.g., first fractionated dose, is at least about 20 fold higher than the subsequent dose, e.g., second dose, e.g., second fractionated dose. In some embodiments, the first dose, e.g., first fractionated dose, is at least about 10 fold higher than the subsequent dose, e.g., second dose, e.g., second fractionated dose. In some embodiments, the first dose, e.g., first fractionated dose, is at least about 2 fold higher than the subsequent dose, e.g., second dose, e.g., second fractionated dose.

In some embodiments, the first dose, e.g., first fractionated dose is about 40 Gy, and the second dose, e.g., second fractionated dose, is about 2 Gy.

In some embodiments, the first dose, e.g., first fractionated dose is about 4 Gy, and the second dose, e.g., second fractionated dose, is about 2 Gy.

In some embodiments, the first dose, e.g., first fractionated dose is about 22 Gy, and the second dose, e.g., second fractionated dose, is about 2.2 Gy.

In some embodiments, the one or more fractionated doses of radiotherapy, e.g., first, second, third and/or fourth fractionated doses of radiotherapy, are administered 30 days prior to administration of a CAR-expressing cell therapy, e.g., CAR19-expressing cell therapy. In some embodiments, the one or more fractionated doses of radiotherapy, e.g., first, second, third and/or fourth fractionated doses of radiotherapy, are administered within 30 days prior to administration of a CAR-expressing cell therapy, e.g., CAR19-expressing cell therapy.

In some embodiments, the one or more fractionated doses of radiotherapy, e.g., first and second fractionated doses of radiotherapy, are administered within about 30 days of each other, e.g., the second dose is administered in less than 30 days from the administration of the first dose.

In some embodiments, the one or more fractionated doses of radiotherapy, e.g., first and second fractionated doses of radiotherapy, are administered more than about 30 days apart, e.g., the second dose is administered more than 30 days, e.g., from the administration of the first dose.

In some embodiments of any of the methods disclosed herien, the lymphodepleting therapy consists of, e.g., consists essentially of radiotherapy.

In some embodiments of any of the methods disclosed herein, the lymphodepleting therapy comprises radiotherapy. In some embodiments, the lymphodepleting therapy further comprises a chemotherapeutic agent described herein. In some embodiments, when the lymphodepleting therapy comprises radiotherapy and a chemotherapeutic agent, the chemotherapeutic agent is not cyclophosphamide.

In some embodiments of any of the methods disclosed herein, the lymphodepleting therapy comprises radiotherapy and a chemotherapeutic agent. In some embodiments, the chemotherapeutic agent is cyclophosphamide. In some embodiments, the cyclophosphamide is administered at a dose of more than 750 mg/m², e.g., about 800, 850, 900, 950, 1000, 1100, 1500 or 2000 mg/m². In some embodiments, the cyclophosphamide is administered at a dose of less than 750 mg/m², e.g., about 700, 650, 600, 550, 500, 400, 300, 200 or 100 mg/m². In some embodiments, the chemotherapeutic agent is cyclophosphamide and the cyclophosphamide is not administered at a does of 750 mg/m².

In some embodiments of a lymphodepleting therapy comprising radiotherapy and cyclophosphamide, the radiotherapy is not administered at a dose of 4 Gy or 2.2 Gy.

### CRS Grading

In some embodiments, CRS can be graded in severity from 1-5 as follows. Grades 1-3 are less than severe CRS. Grades 4-5 are severe CRS. For Grade 1 CRS, only symptomatic treatment is needed (e.g., nausea, fever, fatigue, myalgias, malaise, headache) and symptoms are not life threatening. For Grade 2 CRS, the symptoms require moderate intervention and generally respond to moderate intervention. Subjects having Grade 2 CRS develop hypotension that is responsive to either fluids or one low-dose vasopressor; or they develop grade 2 organ toxicity or mild respiratory symptoms that are responsive to low flow oxygen (<40% oxygen). In Grade 3 CRS subjects, hypotension generally cannot be reversed by fluid therapy or one low-dose vasopressor. These subjects generally require more than low flow oxygen and have grade 3 organ toxicity (e.g., renal or cardiac dysfunction or coagulopathy) and/or grade 4 transaminitis. Grade 3 CRS subjects require more aggressive intervention, e.g., oxygen of 40% or higher, high dose vasopressor(s), and/or multiple vasopressors. Grade 4 CRS subjects suffer from immediately life-threatening symptoms, including grade 4 organ toxicity or a need for mechanical ventilation. Grade 4 CRS subjects generally do not have transaminitis. In Grade 5 CRS subjects, the toxicity causes death. Sets of criteria for grading CRS are provided herein as Table 28B, Table 28C, and Table 28D. Unless otherwise specified, CRS as used herein refers to CRS according to the criteria of Table 28B.

In embodiments, CRS is graded according to Table 28B:

TABLE 28B
CRS grading

| | |
|---|---|
| Gr1 | Supportive care only |
| Gr2 | IV therapies +/− hospitalization. |
| Gr3 | Hypotension requiring IV fluids or low-dose vasoactives or |
| | hypoxemia requiring oxygen, CPAP, or BIPAP. |
| Gr4 | Hypotension requiring high-dose vasoactives or hypoxemia |
| | requiring mechanical ventilation. |
| Gr 5 | Death |

TABLE 28C
CTCAE v 4.0 CRS grading scale

| CRS | |
|---|---|
| grade | Characteristics |
| Grade 1 | Mild; No infusion interruption; No intervention |
| Grade 2 | Infusion interruption indicated but responds promptly to |
| | symptomatic treatment (e.g., antihistamines, NSAIDS, narcotics, |
| | IV fluids); prophylactic medications indicated for <=24 hrs |
| Grade 3 | Prolonged (e.g., not rapidly responsive to symptomatic |
| | medications and/or brief interruption of infusion); recurrence of |
| | symptoms following initial improvement; hospitalization |
| | indicated for clinical sequelae (e.g., renal impairment, |
| | pulmonary infiltrates) |
| Grade 4 | Life threatening consequences; pressor or ventilator support |

TABLE 28D
NCI CRS grading scale

| CRS grade | Characteristics |
|---|---|
| Grade 1 | Symptoms are not life threatening and require symptomatic |
| | treatment only; e.g., fever, nausea, fatigue, headache, |
| | myalgias, malaise |
| Grade 2 | Symptoms require and respond to moderate intervention; |
| | Oxygen requirement <40% or hypotension responsive to fluids |
| | or low dose pressors or Grade 2 organ toxicity |
| Grade 3 | Symptoms require and respond to aggressive intervention; |
| | Oxygen requirement >=40% or Hypotension requiring high |
| | dose or multiple pressors or grade 3 organ toxicity or |
| | grade 4 transaminitis |
| Grade 4 | Life threatening symptoms Requirement for ventilator support |
| | or Grade 4; organ toxicity (excluding transaminitis) |

### CRS Therapies

Therapies for CRS include IL-6 inhibitor or IL-6 receptor (IL-6R) inhibitors (e.g., tocilizumab or siltuximab), bazedoxifene, sgp130 blockers, vasoactive medications, corticosteroids, immunosuppressive agents, and mechanical ventilation. Exemplary therapies for CRS are described in International Application WO2014011984, which is hereby incorporated by reference.

Tocilizumab is a humanized, immunoglobulin Glkappa anti-human IL-6R monoclonal antibody. See, e.g., id. Tocilizumab blocks binding of IL-6 to soluble and membrane bound IL-6 receptors (IL-6Rs) and thus inhibits classical and trans-IL-6 signaling. In embodiments, tocilizumab is administered at a dose of about 4-12 mg/kg, e.g., about 4-8 mg/kg for adults and about 8-12 mg/kg for pediatric subjects, e.g., administered over the course of 1 hour.

In some embodiments, the CRS therapeutic is an inhibitor of IL-6 signalling, e.g., an inhibitor of IL-6 or IL-6 receptor. In one embodiment, the inhibitor is an anti-IL-6 antibody, e.g., an anti-IL-6 chimeric monoclonal antibody such as siltuximab. In other embodiments, the inhibitor comprises a soluble gp130 (sgp130) or a fragment thereof that is capable of blocking IL-6 signalling. In some embodiments, the sgp130 or fragment thereof is fused to a heterologous domain, e.g., an Fc domain, e.g., is a gp130-Fc fusion protein such as FE301. In embodiments, the inhibitor of IL-6 signalling comprises an antibody, e.g., an antibody to the IL-6 receptor, such as sarilumab, olokizumab (CDP6038), elsilimomab, sirukumab (CNTO 136), ALD518/BMS-945429, ARGX-109, or FM101. In some embodiments, the inhibitor of IL-6 signalling comprises a small molecule such as CPSI-2364.

In embodiments, the CAR-expressing cell is administered prior to, concurrently with, or subsequent to administration of one or more therapies for CRS described herein, e.g., one or more of IL-6 inhibitor or IL-6 receptor (IL-6R) inhibitors (e.g., tocilizumab), vasoactive medications, corticosteroids, immunosuppressive agents, or mechanical ventilation. In embodiments, the CAR-expressing cell is administered within 2 weeks (e.g., within 2 or 1 week, or within 14 days, e.g., within 14, 13, 12, 11, 10, 9, 8, 7, 6, 5, 4, 3, 2, 1 day or less) of administration of one or more therapies for CRS described herein, e.g., one or more of IL-6 inhibitors or IL-6 receptor (IL-6R) inhibitors (e.g., tocilizumab), vasoactive medications, corticosteroids, immunosuppressive agents, or mechanical ventilation. In embodiments, the CAR-expressing cell is administered at least 1 day (e.g., at least 1, 2, 3, 4, 5, 6, 7, 8, 9, 10, 11, 12, 13, 14, 15, 16, 17, 18, 19, 20, 1, week, 2 weeks, 3 weeks, 4 weeks, 1 month, 2 months, 3 months, 3 months, or more) before or after administration of one or more therapies for CRS described herein, e.g., one or more of IL-6 inhibitors or IL-6 receptor (IL-6R) inhibitors (e.g., tocilizumab), vasoactive medications, corticosteroids, immunosuppressive agents, or mechanical ventilation.

In embodiments, a subject herein is administered a single dose of an IL-6 inhibitor or IL-6 receptor (IL-6R) inhibitor (e.g., tocilizumab). In embodiments, the subject is administered a plurality of doses (e.g., 2, 3, 4, 5, 6, or more doses) of an IL-6 inhibitor or IL-6 receptor (IL-6R) inhibitor (e.g., tocilizumab).

In some embodiments, the subject at risk of developing severe CRS is administered an anti-IFN-gamma or anti-sIL2Ra therapy, e.g., an antibody molecule directed against IFN-gamma or sIL2Ra.

In embodiments, for a subject who has received a therapeutic antibody molecule such as blinatumomab and who has CRS or is at risk of developing CRS, the therapeutic antibody molecule is administered at a lower dose and/or a lower frequency, or administration of the therapeutic antibody molecule is halted.

In embodiments, a subject who has CRS or is at risk of developing CRS is treated with a fever reducing medication such as acetaminophen.

In embodiments, a subject herein is administered or provided one or more therapies for CRS described herein, e.g., one or more of IL-6 inhibitors or IL-6 receptor (IL-6R) inhibitors (e.g., tocilizumab), vasoactive medications, corticosteroids, immunosuppressive agents, or mechanical ventilation, in any combination, e.g., in combination with a CAR-expressing cell described herein.

In embodiments, a subject at risk of developing CRS (e.g., severe CRS) (e.g., identified as having a high risk status for developing severe CRS) is administered one or more therapies for CRS described herein, e.g., one or more of IL-6 inhibitor or IL-6 receptor (IL-6R) inhibitors (e.g., tocilizumab), vasoactive medications, corticosteroids, immunosuppressive agents, or mechanical ventilation, in any combination, e.g., in combination with a CAR-expressing cell described herein.

In embodiments, a subject herein (e.g., a subject at risk of developing severe CRS or a subject identified as at risk of developing severe CRS) is transferred to an intensive care unit. In some embodiments, a subject herein (e.g., a subject at risk of developing severe CRS or a subject identified as at risk of developing severe CRS) is monitored for one ore more symptoms or conditions associated with CRS, such as fever, elevated heart rate, coagulopathy, MODS (multiple organ dysfunction syndrome), cardiovascular dysfunction, distributive shock, cardiomyopathy, hepatic dysfunction, renal dysfunction, encephalopathy, clinical seizures, respiratory failure, or tachycardia. In some embodiments, the methods herein comprise administering a therapy for one of the symptoms or conditions associated with CRS. For instance, in embodiments, e.g., if the subject develops coagulopathy, the method comprises administering cryoprecipitate. In some embodiments, e.g., if the subject develops cardiovascular dysfunction, the method comprises administering vasoactive infusion support. In some embodiments, e.g., if the subject develops distributive shock, the method comprises administering alpha-agonist therapy. In some embodiments, e.g., if the subject develops cardiomyopathy, the method comprises administering milrinone therapy. In some embodiments, e.g., if the subject develops respiratory failure, the method comprises performing mechanical ventilation (e.g., invasive mechanical ventilation or noninvasive mechanical ventilation). In some embodiments, e.g., if the subject develops shock, the method comprises administering crystalloid and/or colloid fluids.

In embodiments, the CAR-expressing cell is administered prior to, concurrently with, or subsequent to administration of one or more therapies for CRS described herein, e.g., one or more of IL-6 inhibitor or IL-6 receptor (IL-6R) inhibitors (e.g., tocilizumab), vasoactive medications, corticosteroids, immunosuppressive agents, or mechanical ventilation. In embodiments, the CAR-expressing cell is administered within 2 weeks (e.g., within 2 or 1 week, or within 14 days, e.g., within 14, 13, 12, 11, 10, 9, 8, 7, 6, 5, 4, 3, 2, 1 day or less) of administration of one or more therapies for CRS described herein, e.g., one or more of IL-6 inhibitors or IL-6 receptor (IL-6R) inhibitors (e.g., tocilizumab), vasoactive medications, corticosteroids, immunosuppressive agents, or mechanical ventilation. In embodiments, the CAR-expressing cell is administered at least 1 day (e.g., at least 1, 2, 3, 4, 5, 6, 7, 8, 9, 10, 11, 12, 13, 14, 15, 16, 17, 18, 19, 20, 1, week, 2 weeks, 3 weeks, 4 weeks, 1 month, 2 months, 3 months, 3 months, or more) before or after administration of one or more therapies for CRS described herein, e.g., one or more of IL-6 inhibitors or IL-6 receptor (IL-6R) inhibitors (e.g., tocilizumab), vasoactive medications, corticosteroids, immunosuppressive agents, or mechanical ventilation.

In embodiments, a subject herein (e.g., a subject at risk of developing severe CRS or a subject identified as at risk of developing severe CRS) is administered a single dose of an IL-6 inhibitor or IL-6 receptor (IL-6R) inhibitor (e.g., tocilizumab). In embodiments, the subject is administered a plurality of doses (e.g., 2, 3, 4, 5, 6, or more doses) of an IL-6 inhibitor or IL-6 receptor (IL-6R) inhibitor (e.g., tocilizumab).

In embodiments, a subject at low or no risk of developing CRS (e.g., severe CRS) (e.g., identified as having a low risk status for developing severe CRS) is not administered a therapy for CRS described herein, e.g., one or more of IL-6 inhibitor or IL-6 receptor (IL-6R) inhibitors (e.g., tocilizumab), vasoactive medications, corticosteroids, immunosuppressive agents, or mechanical ventilation.

In embodiments, a subject is determined to be at high risk of developing severe CRS by using an evaluation or prediction method described herein. In embodiments, a subject is determined to be at low risk of developing severe CRS by using an evaluation or prediction method described herein.

### CD19 Binding Domains and CARs

Provided herein are compositions of matter and methods of use for the treatment of a disease such as cancer using CD19 chimeric antigen receptors (CAR). The methods also include, e.g., administering a CD19 CAR described herein to treat a lymphoma, e.g., Hodgkin lymphoma or NHL (e.g., relapsed/refractory NHL), or a leukemia, e.g., ALL, e.g., B-ALL.

In one aspect, the invention provides a number of chimeric antigen receptors (CAR) comprising an antibody or antibody fragment engineered for specific binding to a CD19 protein. In one aspect, the invention provides a cell (e.g., T cell) engineered to express a CAR, wherein the CAR T cell (“CART”) exhibits an anticancer property. In one aspect a cell is transformed with the CAR and the CAR is expressed on the cell surface. In some embodiments, the cell (e.g., T cell) is transduced with a viral vector encoding a CAR. In some embodiments, the viral vector is a retroviral vector. In some embodiments, the viral vector is a lentiviral vector. In some such embodiments, the cell may stably express the CAR. In another embodiment, the cell (e.g., T cell) is transfected with a nucleic acid, e.g., mRNA, cDNA, DNA, encoding a CAR. In some such embodiments, the cell may transiently express the CAR.

In one aspect, the anti-CD19 protein binding portion of the CAR is a scFv antibody fragment. In one aspect such antibody fragments are functional in that they retain the equivalent binding affinity, e.g., they bind the same antigen with comparable affinity, as the IgG antibody from which it is derived. In one aspect such antibody fragments are functional in that they provide a biological response that can include, but is not limited to, activation of an immune response, inhibition of signal-transduction origination from its target antigen, inhibition of kinase activity, and the like, as will be understood by a skilled artisan. In one aspect, the anti-CD19 antigen binding domain of the CAR is a scFv antibody fragment that is humanized compared to the murine sequence of the scFv from which it is derived. In one aspect, the parental murine scFv sequence is the CAR19 construct provided in PCT publication WO2012/079000 and provided herein as SEQ ID NO:59. In one embodiment, the anti-CD19 binding domain is a scFv described in WO2012/079000 and provided in SEQ ID NO:59, or a sequence at least 95%, e.g., 95-99%, identical thereto. In an embodiment, the anti-CD19 binding domain is part of a CAR construct provided in PCT publication WO2012/079000 and provided herein as SEQ ID NO:58, or a sequence at least 95%, e.g., 95%-99%, identical thereto. In an embodiment, the anti-CD19 binding domain comprises at least one (e.g., 2, 3, 4, 5, or 6) CDRs selected from Table 4 and/or Table 5.

In some aspects, the antibodies of the invention are incorporated into a chimeric antigen receptor (CAR). In one aspect, the CAR comprises the polypeptide sequence provided as SEQ ID NO: 12 in PCT publication WO2012/079000, and provided herein as SEQ ID NO: 58, wherein the scFv domain is substituted by one or more sequences selected from SEQ ID NOS: 1-12. In one aspect, the scFv domains of SEQ ID NOS:1-12 are humanized variants of the scFv domain of SEQ ID NO:59, which is an scFv fragment of murine origin that specifically binds to human CD19. Humanization of this mouse scFv may be desired for the clinical setting, where the mouse-specific residues may induce a human-anti-mouse antigen (HAMA) response in patients who receive CART19 treatment, e.g., treatment with T cells transduced with the CAR19 construct.

In one embodiment, the CD19 CAR comprises an amino acid sequence provided as SEQ ID NO: 12 in PCT publication WO2012/079000. In embodiment, the amino acid sequence is:

MALPVTALLLPLALLLHAARPdiqmtqttsslsaslgdrvtiscrasqdiskylnwyqqkpdgtvklliyhts rlhsgvpsrfsgsgsgtdysltisnleqediatyfcqqgntlpytfgggtkleitggggsggggsggggsevklqesgpglvapsqslsvt ctvsgvslpdygvswirqpprkglewlgviwgsettyynsalksrltiikdnsksqvflkmnslqtddtaiyycakhyyyggsyamd ywgqgtsvtvsstttpaprpptpaptiasqplslrpeacrpaaggavhtrgldfacdiyiwaplagtcgvlllslvitlyckrgrkkllyifk qpfmrpvqttqeedgcscrfpeeeeggcelrvkfsrsadapaykqgqnqlynelnlgrreeydvldkrrgrdpemggkprrknpqeg lynelqkdkmaeayseigmkgerrrgkghdglyqglstatkdtydalhmqalppr (SEQ ID NO: 58), or a sequence substantially homologous thereto.

In embodiment, the amino acid sequence is diqmtqttsslsaslgdrvtiscrasqdiskylnwyqqkpdgtvklliyhtsrlhsgvpsrfsgsgsgtdysltisnleqediaty fcqqgntlpytfgggtkleitggggsggggsggggsevklqesgpglvapsqslsvtctvsgvslpdygvswirqpprkglewlgviw gsettyynsalksrltiikdnsksqvflkmnslqtddtaiyycakhyyyggsyamdywgqgtsvtvsstttpaprpptpaptiasqplsl rpeacrpaaggavhtrgldfacdiyiwaplagtcgvlllslvitlyckrgrkkllyifkqpfmrpvqttqeedgcscrfpeeeeggcelrvk fsrsadapaykqgqnqlynelnlgrreeydvldkrrgrdpemggkprrknpqeglynelqkdkmaeayseigmkgerrrgkghdg lyqglstatkdtydalhmqalppr (SEQ ID NO: 1633), or a sequence substantially homologous thereto.

In one embodiment, the CD19 CAR has the USAN designation TISAGENLECLEUCEL-T. In embodiments, CTL019 is made by a gene modification of T cells is mediated by stable insertion via transduction with a self-inactivating, replication deficient Lentiviral (LV) vector containing the CTL019 transgene under the control of the EF-1 alpha promoter. CTL019 can be a mixture of transgene positive and negative T cells that are delivered to the subject on the basis of percent transgene positive T cells.

In one aspect, the humanized CAR19 comprises the scFv portion provided in SEQ ID NO:1. In one aspect, the humanized CAR19 comprises the scFv portion provided in SEQ ID NO:2. In one aspect, the humanized CAR19 comprises the scFv portion provided in SEQ ID NO:3. In one aspect, the humanized CAR19 comprises the scFv portion provided in SEQ ID NO:4. In one aspect, the humanized CAR19 comprises the scFv portion provided in SEQ ID NO:5. In one aspect, the humanized CAR19 comprises the scFv portion provided in SEQ ID NO:6. In one aspect, the humanized CAR19 comprises the scFv portion provided in SEQ ID NO:7. In one aspect, the humanized CAR19 comprises the scFv portion provided in SEQ ID NO:8. In one aspect, the humanized CAR19 comprises the scFv portion provided in SEQ ID NO:9. In one aspect, the humanized CAR19 comprises the scFv portion provided in SEQ ID NO:10. In one aspect, the humanized CAR19 comprises the scFv portion provided in SEQ ID NO:11. In one aspect, the humanized CAR19 comprises the scFv portion provided in SEQ ID NO:12.

In one aspect, the CARs of the invention combine an antigen binding domain of a specific antibody with an intracellular signaling molecule. For example, in some aspects, the intracellular signaling molecule includes, but is not limited to, CD3-zeta chain, 4-1BB and CD28 signaling modules and combinations thereof. In one aspect, the CD19 CAR comprises a CAR selected from the sequence provided in one or more of SEQ ID NOS: 31-42. In one aspect, the CD19 CAR comprises the sequence provided in SEQ ID NO:31. In one aspect, the CD19 CAR comprises the sequence provided in SEQ ID NO:32. In one aspect, the CD19 CAR comprises the sequence provided in SEQ ID NO:33. In one aspect, the CD19 CAR comprises the sequence provided in SEQ ID NO:34. In one aspect, the CD19 CAR comprises the sequence provided in SEQ ID NO:35. In one aspect, the CD19 CAR comprises the sequence provided in SEQ ID NO:36. In one aspect, the CD19 CAR comprises the sequence provided in SEQ ID NO:37. In one aspect, the CD19 CAR comprises the sequence provided in SEQ ID NO:38. In one aspect, the CD19 CAR comprises the sequence provided in SEQ ID NO:39. In one aspect, the CD19 CAR comprises the sequence provided in SEQ ID NO:40. In one aspect, the CD19 CAR comprises the sequence provided in SEQ ID NO:41. In one aspect, the CD19 CAR comprises the sequence provided in SEQ ID NO:42.

In embodiments, the CAR molecule is a CD19 CAR molecule described herein, e.g., a humanized CAR molecule described herein, e.g., a humanized CD19 CAR molecule of Table 2 or having CDRs as set out in Tables 4 and 5.

In embodiments, the CAR molecule is a CD19 CAR molecule described herein, e.g., a murine CAR molecule described herein, e.g., a murine CD19 CAR molecule of Table 3 or having CDRs as set out in Tables 4 and 5.

In some embodiments, the CAR molecule comprises one, two, and/or three CDRs from the heavy chain variable region and/or one, two, and/or three CDRs from the light chain variable region of the murine or humanized CD19 CAR of Table 4 and 5.

In one embodiment, the antigen binding domain comprises one, two three (e.g., all three) heavy chain CDRs, HC CDR1, HC CDR2 and HC CDR3, from an antibody listed above, and/or one, two, three (e.g., all three) light chain CDRs, LC CDR1, LC CDR2 and LC CDR3, from an antibody listed above. In one embodiment, the antigen binding domain comprises a heavy chain variable region and/or a variable light chain region of an antibody listed or described above.

In an embodiment, the antigen binding domain comprises a humanized antibody or an antibody fragment. In one embodiment, the humanized anti-CD19 binding domain comprises one or more (e.g., all three) light chain complementary determining region 1 (LC CDR1), light chain complementary determining region 2 (LC CDR2), and light chain complementary determining region 3 (LC CDR3) of a murine or humanized anti-CD19 binding domain described herein, and/or one or more (e.g., all three) heavy chain complementary determining region 1 (HC CDR1), heavy chain complementary determining region 2 (HC CDR2), and heavy chain complementary determining region 3 (HC CDR3) of a murine or humanized anti-CD19 binding domain described herein, e.g., a humanized anti-CD19 binding domain comprising one or more, e.g., all three, LC CDRs and one or more, e.g., all three, HC CDRs.

In one embodiment, an antigen binding domain comprises one, two three (e.g., all three) heavy chain CDRs, HC CDR1, HC CDR2 and HC CDR3, from an antibody listed herein, e.g., in Table 2, 4, or 5 and/or one, two, three (e.g., all three) light chain CDRs, LC CDR1, LC CDR2 and LC CDR3, from an antibody listed herein, e.g., in Table 2, 4, or 5. In one embodiment, the antigen binding domain comprises a heavy chain variable region and/or a variable light chain region of an antibody listed or described above.

In an embodiment, the CD19 binding domain (e.g., an scFv) comprises: a light chain variable region comprising an amino acid sequence having at least one, two or three modifications (e.g., substitutions) but not more than 30, 20 or 10 modifications (e.g., substitutions) of an amino acid sequence of a light chain variable region provided in Table 2, or a sequence with 95-99% identity with an amino acid sequence of Table 2; and/or a heavy chain variable region comprising an amino acid sequence having at least one, two or three modifications (e.g., substitutions) but not more than 30, 20 or 10 modifications (e.g., substitutions) of an amino acid sequence of a heavy chain variable region provided in Table 2, or a sequence with 95-99% identity to an amino acid sequence of Table 2. In embodiments, the CD19 binding domain comprises one or more CDRs (e.g., one each of a HC CDR1, HC CDR2, HC CDR3, LC CDR1, LC CDR2, and LC CDR3) of Table 4 or Table 5, or CDRs having one, two, three, four, five, or six modifications (e.g., substitutions) of one or more of the CDRs.

Exemplary anti-CD19 antibody molecules (including antibodies or fragments or conjugates thereof) can include a scFv, CDRs, or VH and VL chains described in Tables 2, 4, or 5. In an embodiment, the CD19-binding antibody molecule comprises: a light chain variable region comprising an amino acid sequence having at least one, two or three modifications (e.g., substitutions) but not more than 30, 20 or 10 modifications (e.g., substitutions) of an amino acid sequence of a light chain variable region provided in Table 2, or a sequence with 95-99% identity with an amino acid sequence of Table 2; and/or a heavy chain variable region comprising an amino acid sequence having at least one, two or three modifications (e.g., substitutions) but not more than 30, 20 or 10 modifications (e.g., substitutions) of an amino acid sequence of a heavy chain variable region provided in Table 2, or a sequence with 95-99% identity to an amino acid sequence of Table 2. In embodiments, the CD19-binding antibody molecule comprises one or more CDRs (e.g., one each of a HC CDR1, HC CDR2, HC CDR3, LC CDR1, LC CDR2, and LC CDR3) of Table 4 or Table 5, or CDRs having one, two, three, four, five, or six modifications (e.g., substitutions) of one or more of the CDRs. The antibody molecule may be, e.g., an isolated antibody molecule.

In some embodiments, the humanized anti-CD19 binding domain comprises a HC CDR1, a HC CDR2, and a HC CDR3 of any heavy chain binding domain amino acid sequences listed in Table 2. In embodiments, the antigen binding domain further comprises a LC CDR1, a LC CDR2, and a LC CDR3. In embodiments, the antigen binding domain comprises a LC CDR1, a LC CDR2, and a LC CDR3 of any light chain binding domain amino acid sequences listed in Table 2.

In some embodiments, the antigen binding domain comprises one, two or all of LC CDR1, LC CDR2, and LC CDR3 of any light chain binding domain amino acid sequences listed in Table 2, and one, two or all of HC CDR1, HC CDR2, and HC CDR3 of any heavy chain binding domain amino acid sequences listed in Table 2.

In some embodiments, the CDRs are defined according to the Kabat numbering scheme, the Chothia numbering scheme, or a combination thereof.

The sequences of humanized CDR sequences of the scFv domains are shown in Table 4 for the heavy chain variable domains and in Table 5 for the light chain variable domains. “ID” stands for the respective SEQ ID NO for each CDR.

In some embodiments, the CD19 binding domain comprises a Kabat HCDR1 having a sequence of DYGVS (SEQ ID NO: 1634), an HCDR2 of Table 4, an HCDR3 of Table 4, an LCDR1 of Table 5, an LCDR2 of Table 5, and an LCDR3 of Table 5.

In one embodiment, the humanized anti-CD19 binding domain comprises a sequence selected from a group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, and SEQ ID NO:12, or a sequence with 95-99% identity thereof. In one embodiment, the nucleic acid sequence encoding the humanized anti-CD19 binding domain comprises a sequence selected from a group consisting of SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:70, SEQ ID NO:71 and SEQ ID NO:72, or a sequence with 95-99% identity thereof.

In one embodiment, the humanized anti-CD19 binding domain is a scFv, and a light chain variable region comprising an amino acid sequence described herein, e.g., in Table 2, is attached to a heavy chain variable region comprising an amino acid sequence described herein, e.g., in Table 2, via a linker, e.g., a linker described herein. In one embodiment, the humanized anti-CD19 binding domain includes a (Gly4-Ser)n linker, wherein n is 1, 2, 3, 4, 5, or 6, e.g., 3 or 4 (SEQ ID NO:53). The light chain variable region and heavy chain variable region of a scFv can be, e.g., in any of the following orientations: light chain variable region-linker-heavy chain variable region or heavy chain variable region-linker-light chain variable region.

In one aspect, the antigen binding domain portion comprises one or more sequence selected from SEQ ID NOS:1-12. In one aspect the humanized CAR is selected from one or more sequence selected from SEQ ID NOS: 31-42. In some aspects, a non-human antibody is humanized, where specific sequences or regions of the antibody are modified to increase similarity to an antibody naturally produced in a human or fragment thereof.

In one embodiment, the anti-CD19 binding domain comprises a murine light chain variable region described herein (e.g., in Table 3) and/or a murine heavy chain variable region described herein (e.g., in Table 3). In one embodiment, the anti-CD19 binding domain is a scFv comprising a murine light chain and a murine heavy chain of an amino acid sequence of Table 3. In an embodiment, the anti-CD19 binding domain (e.g., an scFv) comprises: a light chain variable region comprising an amino acid sequence having at least one, two or three modifications (e.g., substitutions) but not more than 30, 20 or 10 modifications (e.g., substitutions) of an amino acid sequence of a light chain variable region provided in Table 3, or a sequence with 95-99% identity with an amino acid sequence of Table 3; and/or a heavy chain variable region comprising an amino acid sequence having at least one, two or three modifications (e.g., substitutions) but not more than 30, 20 or 10 modifications (e.g., substitutions) of an amino acid sequence of a heavy chain variable region provided in Table 3, or a sequence with 95-99% identity to an amino acid sequence of Table 3. In one embodiment, the anti-CD19 binding domain comprises a sequence of SEQ ID NO:59, or a sequence with 95-99% identity thereof. In one embodiment, the anti-CD19 binding domain is a scFv, and a light chain variable region comprising an amino acid sequence described herein, e.g., in Table 3, is attached to a heavy chain variable region comprising an amino acid sequence described herein, e.g., in Table 3, via a linker, e.g., a linker described herein. In one embodiment, the antigen binding domain includes a (Gly4-Ser)n linker, wherein n is 1, 2, 3, 4, 5, or 6, e.g., 3 or 4 (SEQ ID NO: 53). The light chain variable region and heavy chain variable region of a scFv can be, e.g., in any of the following orientations: light chain variable region-linker-heavy chain variable region or heavy chain variable region-linker-light chain variable region.

In embodiments, the CAR molecule comprises a CD19 inhibitor comprising an antibody or antibody fragment which includes a CD19 binding domain, a transmembrane domain, and an intracellular signaling domain comprising a stimulatory domain, and wherein said CD19 binding domain comprises one or more of (e.g., all three of) light chain complementary determining region 1 (LC CDR1), light chain complementary determining region 2 (LC CDR2), and light chain complementary determining region 3 (LC CDR3) of any CD19 light chain binding domain amino acid sequence listed in Tables 2 or 3, and one or more of (e.g., all three of) heavy chain complementary determining region 1 (HC CDR1), heavy chain complementary determining region 2 (HC CDR2), and heavy chain complementary determining region 3 (HC CDR3) of any CD19 heavy chain binding domain amino acid sequence listed in Tables 2 or 3.

In embodiments, a CD19 CAR comprises light chain variable region listed in Tables 2 or 3 and any heavy chain variable region listed Tables 2 or 3.

In embodiments, the CD19 inhibitor comprises a CD19 binding domain which comprises a sequence selected from a group consisting of SEQ ID NO:1, SEQ ID NO:2, SEQ ID NO:3, SEQ ID NO: 4, SEQ ID NO:5, SEQ ID NO:6, SEQ ID NO:7, SEQ ID NO:8, SEQ ID NO:9, SEQ ID NO:10, SEQ ID NO:11 and SEQ ID NO:12, or a sequence with 95-99% identity thereof. In embodiments, the CD19 CAR comprises a polypeptide of SEQ ID NO:58.

In one embodiment, the CAR molecule comprises an anti-CD19 binding domain comprising one or more (e.g., all three) light chain complementary determining region 1 (LC CDR1), light chain complementary determining region 2 (LC CDR2), and light chain complementary determining region 3 (LC CDR3) of an anti-CD19 binding domain described herein, and one or more (e.g., all three) heavy chain complementary determining region 1 (HC CDR1), heavy chain complementary determining region 2 (HC CDR2), and heavy chain complementary determining region 3 (HC CDR3) of an anti-CD19 binding domain described herein, e.g., an anti-CD19 binding domain comprising one or more, e.g., all three, LC CDRs and one or more, e.g., all three, HC CDRs. In one embodiment, the anti-CD19 binding domain comprises one or more (e.g., all three) heavy chain complementary determining region 1 (HC CDR1), heavy chain complementary determining region 2 (HC CDR2), and heavy chain complementary determining region 3 (HC CDR3) of an anti-CD19 binding domain described herein, e.g., the anti-CD19 binding domain has two variable heavy chain regions, each comprising a HC CDR1, a HC CDR2 and a HC CDR3 described herein.

In one aspect, the anti-CD19 binding domain is characterized by particular functional features or properties of an antibody or antibody fragment. For example, in one aspect, the portion of a CAR composition of the invention that comprises an antigen binding domain specifically binds human CD19. In one aspect, the invention relates to an antigen binding domain comprising an antibody or antibody fragment, wherein the antibody binding domain specifically binds to a CD19 protein or fragment thereof, wherein the antibody or antibody fragment comprises a variable light chain and/or a variable heavy chain that includes an amino acid sequence of SEQ ID NO: 1-12 or SEQ ID NO:59. In one aspect, the antigen binding domain comprises an amino acid sequence of an scFv selected from SEQ ID NOs: 1-12 or SEQ ID NO:59. In certain aspects, the scFv is contiguous with and in the same reading frame as a leader sequence. In one aspect the leader sequence is the polypeptide sequence provided as SEQ ID NO:13.

In one aspect, the portion of the CAR comprising the antigen binding domain comprises an antigen binding domain that targets CD19. In one aspect, the antigen binding domain targets human CD19. In one aspect, the antigen binding domain of the CAR has the same or a similar binding specificity as, or includes, the FMC63 scFv fragment described in Nicholson et al. Mol. Immun. 34 (16-17): 1157-1165 (1997). In one aspect, the portion of the CAR comprising the antigen binding domain comprises an antigen binding domain that targets a B-cell antigen, e.g., a human B-cell antigen. A CD19 antibody molecule can be, e.g., an antibody molecule (e.g., a humanized anti-CD19 antibody molecule) described in WO2014/153270, which is incorporated herein by reference in its entirety. WO2014/153270 also describes methods of assaying the binding and efficacy of various CART constructs.

In some embodiments, the CD19 CAR comprises an antigen binding domain derived from (e.g., comprises an amino acid sequence of) an anti-CD19 antibody (e.g., an anti-CD19 mono- or bispecific antibody) or a fragment or conjugate thereof. In one embodiment, the anti-CD19 antibody is a humanized antigen binding domain as described in WO2014/153270 (e.g., Table 3 of WO2014/153270) incorporated herein by reference, or a conjugate thereof. Other exemplary anti-CD19 antibodies or fragments or conjugates thereof, include but are not limited to, a bispecific T cell engager that targets CD19 (e.g., blinatumomab), SAR3419 (Sanofi), MEDI-551 (Medlmmune LLC), Combotox, DT2219ARL (Masonic Cancer Center), MOR-208 (also called XmAb-5574; MorphoSys), XmAb-5871 (Xencor), MDX-1342 (Bristol-Myers Squibb), SGN-CD19A (Seattle Genetics), and AFM11 (Affimed Therapeutics). See, e.g., Hammer. MAbs. 4.5(2012): 571-77. Blinatomomab is a bispecific antibody comprised of two scFvs—one that binds to CD19 and one that binds to CD3. Blinatomomab directs T cells to attack cancer cells. See, e.g., Hammer et al.; Clinical Trial Identifier No. NCT00274742 and NCT01209286. MEDI-551 is a humanized anti-CD19 antibody with a Fc engineered to have enhanced antibody-dependent cell-mediated cytotoxicity (ADCC). See, e.g., Hammer et al.; and Clinical Trial Identifier No. NCT01957579. Combotox is a mixture of immunotoxins that bind to CD19 and CD22. The immunotoxins are made up of scFv antibody fragments fused to a deglycosylated ricin A chain. See, e.g., Hammer et al.; and Herrera et al. J. Pediatr. Hematol. Oncol. 31.12(2009):936-41; Schindler et al. Br. J. Haematol. 154.4(2011):471-6. DT2219ARL is a bispecific immunotoxin targeting CD19 and CD22, comprising two scFvs and a truncated diphtheria toxin. See, e.g., Hammer et al.; and Clinical Trial Identifier No. NCT00889408. SGN-CD19A is an antibody-drug conjugate (ADC) comprised of an anti-CD19 humanized monoclonal antibody linked to a synthetic cytotoxic cell-killing agent, monomethyl auristatin F (MMAF). See, e.g., Hammer et al.; and Clinical Trial Identifier Nos. NCT01786096 and NCT01786135. SAR3419 is an anti-CD19 antibody-drug conjugate (ADC) comprising an anti-CD19 humanized monoclonal antibody conjugated to a maytansine derivative via a cleavable linker. See. e.g., Younes et al. J. Clin. Oncol. 30.2(2012): 2776-82; Hammer et al.; Clinical Trial Identifier No. NCT00549185; and Blanc et al. Clin Cancer Res. 2011; 17:6448-58. XmAb-5871 is an Fc-engineered, humanized anti-CD19 antibody. See, e.g., Hammer et al. MDX-1342 is a human Fc-engineered anti-CD19 antibody with enhanced ADCC. See, e.g., Hammer et al. In embodiments, the antibody molecule is a bispecific anti-CD19 and anti-CD3 molecule. For instance, AFM11 is a bispecific antibody that targets CD19 and CD3. See, e.g., Hammer et al.; and Clinical Trial Identifier No. NCT02106091. In some embodiments, an anti-CD19 antibody described herein is conjugated or otherwise bound to a therapeutic agent, e.g., a chemotherapeutic agent, peptide vaccine (such as that described in Izumoto et al. 2008 J Neurosurg 108:963-971), immunosuppressive agent, or immunoablative agent, e.g., cyclosporin, azathioprine, methotrexate, mycophenolate, FK506, CAMPATH, anti-CD3 antibody, cytoxin, fludarabine, rapamycin, mycophenolic acid, steroid, FR901228, or cytokine.

In one embodiment, an antigen binding domain against CD19 is an antigen binding portion, e.g., CDRs, of an antigen binding domain described in a Table herein. In one embodiment, a CD19 antigen binding domain can be from any CD19 CAR, e.g., LG-740; U.S. Pat. Nos. 8,399,645; 7,446,190; Xu et al., Leuk Lymphoma. 2013 54(2):255-260(2012); Cruz et al., Blood 122(17):2965-2973 (2013); Brentjens et al., Blood, 118(18):4817-4828 (2011); Kochenderfer et al., Blood 116(20):4099-102 (2010); Kochenderfer et al., Blood 122 (25):4129-39(2013); and 16th Annu Meet Am Soc Gen Cell Ther (ASGCT) (May 15-18, Salt Lake City) 2013, Abst 10, each of which is herein incorporated by reference in its entirety.

In embodiments, the CAR molecule comprises a CD19 CAR molecule described herein, e.g., a CD19 CAR molecule described in US-2015-0283178-A1, e.g., CTL019. In embodiments, the CD19 CAR comprises an amino acid, or has a nucleotide sequence shown in US-2015-0283178-A1, incorporated herein by reference.

In one aspect, the invention provides a cell (e.g., T cell) engineered to express a chimeric antigen receptor (CAR), wherein the CAR-expressing cell, e.g., CAR T cell (“CART”) exhibits an anticancer property. A suitable antigen is CD19. In one aspect, the antigen binding domain of the CAR comprises a partially humanized anti-CD19 antibody fragment. In one aspect, the antigen binding domain of the CAR comprises a partially humanized anti-CD19 antibody fragment comprising an scFv. Accordingly, the invention provides (among other things) a CD19-CAR that comprises a humanized anti-CD19 binding domain and is engineered into an immune effector cell, e.g., a T cell or an NK cell, and methods of their use for adoptive therapy.

In one aspect, the CAR, e.g., CD19-CAR comprises at least one intracellular domain selected from the group of a CD137 (4-1BB) signaling domain, a CD28 signaling domain, a CD3zeta signal domain, and any combination thereof. In one aspect, the CAR, e.g., CD19-CAR comprises at least one intracellular signaling domain is from one or more co-stimulatory molecule(s) other than a CD137 (4-1BB) or CD28.

### Exemplary CD19 CAR Constructs

Of the CD19 CAR constructs described in International Application WO2014/153270, certain sequences are reproduced herein. It is understood that the sequences in this section can also be used in the context of other CARs, e.g., as described herein, e.g., BCMA CARs.

The sequences of the murine scFv fragments (SEQ ID NOS: 98, 109, 111 and 114) are provided below in Table 3. Full CAR constructs were generated using SEQ ID NOs: 98, 109, 111 and 114 with additional sequences, SEQ ID NOs: 13-17, shown below, to generate full CAR constructs with SEQ ID NOs: 58, 110, 112, 113 and 115.

The sequences of the humanized scFv fragments (SEQ ID NOS: 1-12) are provided below in Table 2. Full CAR constructs were generated using SEQ ID NOs: 1-12 with additional sequences, SEQ ID NOs: 13-17, shown below, to generate full CAR constructs with SEQ ID NOs: 31-42.

leader (amino acid sequence)
(SEQ ID NO: 13)
MALPVTALLLPLALLLHAARP
leader (nucleic acid sequence)
(SEQ ID NO: 54)
ATGGCCCTGCCTGTGACAGCCCTGCTGCTGCCTCTGGCTCTGCTGCTGCATGCCGCT
AGACCC
CD8 hinge (amino acid sequence)
(SEQ ID NO: 14)
TTTPAPRPPTPAPTIASQPLSLRPEACRPAAGGAVHTRGLDFACD
CD8 hinge (nucleic acid sequence)
(SEQ ID NO: 55)
ACCACGACGCCAGCGCCGCGACCACCAACACCGGCGCCCACCATCGCGTCGCAGCC
CCTGTCCCTGCGCCCAGAGGCGTGCCGGCCAGCGGCGGGGGGCGCAGTGCACACGA
GGGGGCTGGACTTCGCCTGTGAT
CD8 transmembrane (amino acid sequence)
(SEQ ID NO: 15)
IYIWAPLAGTCGVLLLSLVITLYC
transmembrane (nucleic acid sequence)
(SEQ ID NO: 56)
ATCTACATCTGGGCGCCCTTGGCCGGGACTTGTGGGGTCCTTCTCCTGTCACTGGTT
ATCACCCTTTACTGC
4-1BB Intracellular domain (amino acid sequence)
(SEQ ID NO: 16)
KRGRKKLLYIFKQPFMRPVQTTQEEDGCSCRFPEEEEGGCEL
4-1BB Intracellular domain (nucleic acid sequence)
(SEQ ID NO: 60)
AAACGGGGCAGAAAGAAACTCCTGTATATATTCAAACAACCATTTATGAGACCAGT
ACAAACTACTCAAGAGGAAGATGGCTGTAGCTGCCGATTTCCAGAAGAAGAAGAA
GGAGGATGTGAACTG
CD3 zeta domain (amino acid sequence)
(SEQ ID NO: 17)
RVKFSRSADAPAYKQGQNQLYNELNLGRREEYDVLDKRRGRDPEMGGKPRRKNPQEG
LYNELQKDKMAEAYSEIGMKGERRRGKGHDGLYQGLSTATKDTYDALHMQALPPR
CD3 zeta (nucleic acid sequence)
(SEQ ID NO: 101)
AGAGTGAAGTTCAGCAGGAGCGCAGACGCCCCCGCGTACAAGCAGGGCCAGAACC
AGCTCTATAACGAGCTCAATCTAGGACGAAGAGAGGAGTACGATGTTTTGGACAAG
AGACGTGGCCGGGACCCTGAGATGGGGGGAAAGCCGAGAAGGAAGAACCCTCAGG
AAGGCCTGTACAATGAACTGCAGAAAGATAAGATGGCGGAGGCCTACAGTGAGAT
TGGGATGAAAGGCGAGCGCCGGAGGGGCAAGGGGCACGATGGCCTTTACCAGGGT
CTCAGTACAGCCACCAAGGACACCTACGACGCCCTTCACATGCAGGCCCTGCCCCC
TCGC
CD3 zeta domain (amino acid sequence;
NCBI Reference Sequence NM_000734.3)
(SEQ ID NO: 43)
RVKFSRSADAPAYQQGQNQLYNELNLGRREEYDVLDKRRGRDPEMGGKPRRKNPQEG
LYNELQKDKMAEAYSEIGMKGERRRGKGHDGLYQGLSTATKDTYDALHMQALPPR
CD3 zeta (nucleic acid sequence;
NCBI Reference Sequence NM_000734.3);
(SEQ ID NO: 44)
AGAGTGAAGTTCAGCAGGAGCGCAGACGCCCCCGCGTACCAGCAGGGCCAG
AACCAGCTCTATAACGAGCTCAATCTAGGACGAAGAGAGGAGTACGATGTTT
TGGACAAGAGACGTGGCCGGGACCCTGAGATGGGGGGAAAGCCGAGAAGGA
AGAACCCTCAGGAAGGCCTGTACAATGAACTGCAGAAAGATAAGATGGCGG
AGGCCTACAGTGAGATTGGGATGAAAGGCGAGCGCCGGAGGGGCAAGGGGC
ACGATGGCCTTTACCAGGGTCTCAGTACAGCCACCAAGGACACCTACGACGC
CCTTCACATGCAGGCCCTGCCCCCTCGC
CD28 domain (amino acid sequence, SEQ ID NO: 1317)
RSKRSRLLHSDYMNMTPRRPGPTRKHYQPYAPPRDFAAYRS
CD28 domain (nucleotide sequence, SEQ ID NO: 1318)
AGGAGTAAGAGGAGCAGGCTCCTGCACAGTGACTACATGAACATGACTCCCCGCCG
CCCCGGGCCCACCCGCAAGCATTACCAGCCCTATGCCCCACCACGCGACTTCGCAG
CCTATCGCTCC
Wild-type ICOS domain (amino acid sequence,
SEQ ID NO: 1319)
TKKKYSSSVHDPNGEYMFMRAVNTAKKSRLTDVTL
Wild-type ICOS domain (nucleotide sequence,
SEQ ID NO: 1320)
ACAAAAAAGAAGTATTCATCCAGTGTGCACGACCCTAACGGTGAATACATGTTCAT
GAGAGCAGTGAACACAGCCAAAAAATCCAGACTCACAGATGTGACCCTA
Y to F mutant ICOS domain (amino acid sequence,
SEQ ID NO: 1321)
TKKKYSSSVHDPNGEFMFMRAVNTAKKSRLTDVTL
IgG4 Hinge (amino acid sequence)
(SEQ ID NO: 102)
ESKYGPPCPPCPAPEFLGGPSVFLFPPKPKDTLMISRTPEVTCVVVDVSQEDPEVQFNWY
VDGVEVHNAKTKPREEQFNSTYRVVSVLTVLHQDWLNGKEYKCKVSNKGLPSSIEKTI
SKAKGQPREPQVYTLPPSQEEMTKNQVSLTCLVKGFYPSDIAVEWESNGQPENNYKTTP
PVLDSDGSFFLYSRLTVDKSRWQEGNVFSCSVMHEALHNHYTQKSLSLSLGKM
IgG4 Hinge (nucleotide sequence)
(SEQ ID NO: 103)
GAGAGCAAGTACGGCCCTCCCTGCCCCCCTTGCCCTGCCCCCGAGTTCCTGGGCGGA
CCCAGCGTGTTCCTGTTCCCCCCCAAGCCCAAGGACACCCTGATGATCAGCCGGACC
CCCGAGGTGACCTGTGTGGTGGTGGACGTGTCCCAGGAGGACCCCGAGGTCCAGTT
CAACTGGTACGTGGACGGCGTGGAGGTGCACAACGCCAAGACCAAGCCCCGGGAG
GAGCAGTTCAATAGCACCTACCGGGTGGTGTCCGTGCTGACCGTGCTGCACCAGGA
CTGGCTGAACGGCAAGGAATACAAGTGTAAGGTGTCCAACAAGGGCCTGCCCAGCA
GCATCGAGAAAACCATCAGCAAGGCCAAGGGCCAGCCTCGGGAGCCCCAGGTGTA
CACCCTGCCCCCTAGCCAAGAGGAGATGACCAAGAACCAGGTGTCCCTGACCTGCC
TGGTGAAGGGCTTCTACCCCAGCGACATCGCCGTGGAGTGGGAGAGCAACGGCCAG
CCCGAGAACAACTACAAGACCACCCCCCCTGTGCTGGACAGCGACGGCAGCTTCTT
CCTGTACAGCCGGCTGACCGTGGACAAGAGCCGGTGGCAGGAGGGCAACGTCTTTA
GCTGCTCCGTGATGCACGAGGCCCTGCACAACCACTACACCCAGAAGAGCCTGAGC
CTGTCCCTGGGCAAGATG

The CAR scFv fragments were then cloned into lentiviral vectors to create a full length CAR construct in a single coding frame, and using the EF1 alpha promoter for expression (SEQ ID NO: 100).

EF-1 alpha promoter

(SEQ ID NO: 100)

CGTGAGGCTCCGGTGCCCGTCAGTGGGCAGAGCGCACATCGCCCACAGTC
CCCGAGAAGTTGGGGGGAGGGGTCGGCAATTGAACCGGTGCCTAGAGAAG
GTGGCGCGGGGTAAACTGGGAAAGTGATGTCGTGTACTGGCTCCGCCTTT
TTCCCGAGGGTGGGGGAGAACCGTATATAAGTGCAGTAGTCGCCGTGAAC
GTTCTTTTTCGCAACGGGTTTGCCGCCAGAACACAGGTAAGTGCCGTGTG
TGGTTCCCGCGGGCCTGGCCTCTTTACGGGTTATGGCCCTTGCGTGCCTT
GAATTACTTCCACCTGGCTGCAGTACGTGATTCTTGATCCCGAGCTTCGG
GTTGGAAGTGGGTGGGAGAGTTCGAGGCCTTGCGCTTAAGGAGCCCCTTC
GCCTCGTGCTTGAGTTGAGGCCTGGCCTGGGCGCTGGGGCCGCCGCGTGC
GAATCTGGTGGCACCTTCGCGCCTGTCTCGCTGCTTTCGATAAGTCTCTA
GCCATTTAAAATTTTTGATGACCTGCTGCGACGCTTTTTTTCTGGCAAGA
TAGTCTTGTAAATGCGGGCCAAGATCTGCACACTGGTATTTCGGTTTTTG
GGGCCGCGGGCGGCGACGGGGCCCGTGCGTCCCAGCGCACATGTTCGGCG
AGGCGGGGCCTGCGAGCGCGGCCACCGAGAATCGGACGGGGGTAGTCTCA
AGCTGGCCGGCCTGCTCTGGTGCCTGGCCTCGCGCCGCCGTGTATCGCCC
CGCCCTGGGCGGCAAGGCTGGCCCGGTCGGCACCAGTTGCGTGAGCGGAA
AGATGGCCGCTTCCCGGCCCTGCTGCAGGGAGCTCAAAATGGAGGACGCG
GCGCTCGGGAGAGCGGGCGGGTGAGTCACCCACACAAAGGAAAAGGGCCT
TTCCGTCCTCAGCCGTCGCTTCATGTGACTCCACGGAGTACCGGGCGCCG
TCCAGGCACCTCGATTAGTTCTCGAGCTTTTGGAGTACGTCGTCTTTAGG
TTGGGGGGAGGGGTTTTATGCGATGGAGTTTCCCCACACTGAGTGGGTGG
AGACTGAAGTTAGGCCAGCTTGGCACTTGATGTAATTCTCCTTGGAATTT
GCCCTTTTTGAGTTTGGATCTTGGTTCATTCTCAAGCCTCAGACAGTGGT
TCAAAGTTTTTTTCTTCCATTTCAGGTGTCGTGA.

In embodiments, these clones contain a Q/K residue change in the signal domain of the co-stimulatory domain derived from 4-1BB.

In one aspect, the anti-CD19 binding domain, e.g., humanized scFv, portion of a CAR of the invention is encoded by a transgene whose sequence has been codon optimized for expression in a mammalian cell. In one aspect, entire CAR construct of the invention is encoded by a transgene whose entire sequence has been codon optimized for expression in a mammalian cell. Codon optimization refers to the discovery that the frequency of occurrence of synonymous codons (i.e., codons that code for the same amino acid) in coding DNA is biased in different species. Such codon degeneracy allows an identical polypeptide to be encoded by a variety of nucleotide sequences. A variety of codon optimization methods is known in the art, and include, e.g., methods disclosed in at least U.S. Pat. Nos. 5,786,464 and 6,114,148.

The present disclosure encompasses, but is not limited to, a recombinant DNA construct comprising sequences encoding a CAR, wherein the CAR comprises an antibody or antibody fragment that binds specifically to CD19, wherein the sequence of the antibody fragment is contiguous with and in the same reading frame as a nucleic acid sequence encoding an intracellular signaling domain. The intracellular signaling domain can comprise a costimulatory signaling domain and/or a primary signaling domain, e.g., a zeta chain. The costimulatory signaling domain refers to a portion of the CAR comprising at least a portion of the intracellular domain of a costimulatory molecule. In one embodiment, the antigen binding domain is a murine antibody or antibody fragment described herein. In one embodiment, the antigen binding domain is a humanized antibody or antibody fragment.

In specific aspects, a CAR construct of the invention comprises a scFv domain selected from the group consisting of SEQ ID NOS:1-12 or an scFV domain of SEQ ID NO:59, wherein the scFv may be preceded by an optional leader sequence such as provided in SEQ ID NO: 13, and followed by an optional hinge sequence such as provided in SEQ ID NO:14 or SEQ ID NO:45 or SEQ ID NO:47 or SEQ ID NO:49, a transmembrane region such as provided in SEQ ID NO:15, an intracellular signalling domain that includes SEQ ID NO:16 or SEQ ID NO:51 and a CD3 zeta sequence that includes SEQ ID NO:17 or SEQ ID NO:43, wherein the domains are contiguous with and in the same reading frame to form a single fusion protein.

Also included in the invention (among other things) is a nucleotide sequence that encodes the polypeptide of each of the scFv fragments selected from the group consisting of SEQ IS NO:1, SEQ ID NO:2, SEQ ID NO:3, SEQ ID NO:4, SEQ ID NO:5, SEQ IS NO:6, SEQ ID NO:7, SEQ ID NO:8, SEQ ID NO:9, SEQ ID NO:10, SEQ ID NO:11, SEQ ID NO:12 and SEQ ID NO:59. Also included in the invention (among other things) is a nucleotide sequence that encodes the polypeptide of each of the scFv fragments selected from the group consisting of SEQ IS NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12 and SEQ ID NO:59, and each of the domains of SEQ ID NOS: 13-17, plus an encoded CD19 CAR fusion protein of the invention. In one aspect an exemplary CD19 CAR constructs comprise an optional leader sequence, an extracellular antigen binding domain, a hinge, a transmembrane domain, and an intracellular stimulatory domain. In one aspect an exemplary CD19 CAR construct comprises an optional leader sequence, an extracellular antigen binding domain, a hinge, a transmembrane domain, an intracellular costimulatory domain and an intracellular stimulatory domain. In some embodiments, specific CD19 CAR constructs containing humanized scFv domains of the invention are provided as SEQ ID NOS: 31-42, or a murine scFv domain as provided as SEQ ID NO:59.

In one aspect the nucleic acid sequence of a CAR construct of the invention is selected from one or more of SEQ ID NOS:85-96. In one aspect the nucleic acid sequence of a CAR construct is SEQ ID NO:85. In one aspect the nucleic acid sequence of a CAR construct is SEQ ID NO:86. In one aspect the nucleic acid sequence of a CAR construct is SEQ ID NO:87. In one aspect the nucleic acid sequence of a CAR construct is SEQ ID NO:88. In one aspect the nucleic acid sequence of a CAR construct is SEQ ID NO:89. In one aspect the nucleic acid sequence of a CAR construct is SEQ ID NO:90. In one aspect the nucleic acid sequence of a CAR construct is SEQ ID NO:91. In one aspect the nucleic acid sequence of a CAR construct is SEQ ID NO:92. In one aspect the nucleic acid sequence of a CAR construct is SEQ ID NO:93. In one aspect the nucleic acid sequence of a CAR construct is SEQ ID NO:94. In one aspect the nucleic acid sequence of a CAR construct is SEQ ID NO:95. In one aspect the nucleic acid sequence of a CAR construct is SEQ ID NO:96. In one aspect the nucleic acid sequence of a CAR construct is SEQ ID NO:97. In one aspect the nucleic acid sequence of a CAR construct is SEQ ID NO:98. In one aspect the nucleic acid sequence of a CAR construct is SEQ ID NO:99.

Full-length CAR sequences are also provided herein as SEQ ID NOS: 31-42 and 58, as shown in Table 2 (e.g., CTL119) and Table 3 (e.g., CTL019).

An exemplary leader sequence is provided as SEQ ID NO: 13. An exemplary hinge/spacer sequence is provided as SEQ ID NO: 14 or SEQ ID NO:45 or SEQ ID NO:47 or SEQ ID NO:49. An exemplary transmembrane domain sequence is provided as SEQ ID NO:15. An exemplary sequence of the intracellular signaling domain of the 4-1BB protein is provided as SEQ ID NO: 16. An exemplary sequence of the intracellular signaling domain of CD27 is provided as SEQ ID NO:51. An exemplary CD3zeta domain sequence is provided as SEQ ID NO: 17 or SEQ ID NO:43. These sequences may be used, e.g., in combination with an scFv that recognizes one or more of CD19, CD10, CD20, CD22, CD34, CD123, FLT-3, or ROR1.

Exemplary sequences of various scFv fragments and other CAR components are provided herein. It is noted that these CAR components (e.g., of SEQ ID NO: 121, or a sequence of Table 2, 3, 6, 11A, 11B, 16, or 25) without a leader sequence (e.g., without the amino acid sequence of SEQ ID NO: 13 or a nucleotide sequence of SEQ ID NO: 54), are also provided herein.

In embodiments, the CAR sequences described herein contain a Q/K residue change in the signal domain of the co-stimulatory domain derived from CD3zeta chain.

In one aspect, the present invention encompasses a recombinant nucleic acid construct comprising a nucleic acid molecule encoding a CAR, wherein the nucleic acid molecule comprises the nucleic acid sequence encoding an anti-CD19 binding domain, e.g., described herein, that is contiguous with and in the same reading frame as a nucleic acid sequence encoding an intracellular signaling domain. In one aspect, the anti-CD19 binding domain is selected from one or more of SEQ ID NOS:1-12 and 58. In one aspect, the anti-CD19 binding domain is encoded by a nucleotide residues 64 to 813 of the sequence provided in one or more of SEQ ID NOS:61-72 and 97. In one aspect, the anti-CD19 binding domain is encoded by a nucleotide residues 64 to 813 of SEQ ID NO:61. In one aspect, the anti-CD19 binding domain is encoded by a nucleotide residues 64 to 813 of SEQ ID NO:62. In one aspect, the anti-CD19 binding domain is encoded by a nucleotide residues 64 to 813 of SEQ ID NO:63. In one aspect, the anti-CD19 binding domain is encoded by a nucleotide residues 64 to 813 of SEQ ID NO:64. In one aspect, the anti-CD19 binding domain is encoded by a nucleotide residues 64 to 813 of SEQ ID NO:65. In one aspect, the anti-CD19 binding domain is encoded by a nucleotide residues 64 to 813 of SEQ ID NO:66. In one aspect, the anti-CD19 binding domain is encoded by a nucleotide residues 64 to 813 of SEQ ID NO:67. In one aspect, the anti-CD19 binding domain is encoded by a nucleotide residues 64 to 813 of SEQ ID NO:68. In one aspect, the anti-CD19 binding domain is encoded by a nucleotide residues 64 to 813 of SEQ ID NO:69. In one aspect, the anti-CD19 binding domain is encoded by a nucleotide residues 64 to 813 of SEQ ID NO:70. In one aspect, the anti-CD19 binding domain is encoded by a nucleotide residues 64 to 813 of SEQ ID NO:71. In one aspect, the anti-CD19 binding domain is encoded by a nucleotide residues 64 to 813 of SEQ ID NO:72.

TABLE 2
Humanized CD19 CAR Constructs

| | | |
|---|---|---|
| | SEQ | |
| Name | ID | Sequence |

CAR 1

| | | |
|---|---|---|
| CAR1 | 1 | EIVMTQSPATLSLSPGERATLSCRASQDISKYLNWYQQKPGQA |
| scFv | | PRLLIYHTSRLHSGIPARFSGSGSGTDYTLTISSLQPEDFAVYFC |
| domain | | QQGNTLPYTFGQGTKLEIKGGGGSGGGGSGGGGSQVQLQESG |
| | | PGLVKPSETLSLTCTVSGVSLPDYGVSWIRQPPGKGLEWIGVI |
| | | WGSETTYYSSSLKSRVTISKDNSKNQVSLKLSSVTAADTAVYY |
| | | CAKHYYYGGSYAMDYWGQGTLVTVSS |
| 103101 | 61 | atggccctccctgtcaccgccctgctgcttccgctggctcttctgctccacgccgctcggcccgaaa |
| CAR1 | | ttgtgatgacccagtcacccgccactcttagcctttcacccggtgagcgcgcaaccctgtcttgcag |
| Soluble | | agcctcccaagacatctcaaaataccttaattggtatcaacagaagcccggacaggctcctcgcctt |
| scFv-nt | | ctgatctaccacaccagccggctccattctggaatccctgccaggttcagcggtagcggatctggg |
| | | accgactacaccctcactatcagctcactgcagccagaggacttcgctgtctatttctgtcagcaagg |
| | | gaacaccctgccctacacctttggacagggcaccaagctcgagattaaaggtggaggtggcagcg |
| | | gaggaggtgggtccggcggtggaggaagccaggtccaactccaagaaagcggaccgggtcttg |
| | | tgaagccatcagaaactctttcactgacttgtactgtgagcggagtgtctctccccgattacggggtgt |
| | | cttggatcagacagccaccggggaagggtctggaatggattggagtgatttggggctctgagacta |
| | | cttactactcttcatccctcaagtcacgcgtcaccatctcaaaggacaactctaagaatcaggtgtcac |
| | | tgaaactgtcatctgtgaccgcagccgacaccgccgtgtactattgcgctaagcattactattatggc |
| | | gggagctacgcaatggattactggggacagggtactctggtcaccgtgtccagccaccaccatcat |
| | | caccatcaccat |
| 103101 | 73 | MALPVTALLLPLALLLHAARPeivmtqspatlslspgeratlscrasqdiskyl |
| CAR1 | | nwyqqkpgqaprlliyhtsrlhsgiparfsgsgsgtdytltisslqpedfavyfcqqgntlpytfgq |
| Soluble | | gtkleikggggsggggsggggsqvqlqesgpglvkpsetlsltctvsgvslpdygvswirqppg |
| scFv-aa | | kglewigviwgsettyyssslksrvtiskdnsknqvslklssvtaadtavyycakhyyyggsya |
| | | mdywgqgtlvtvsshhhhhhhh |
| 104875 | 85 | atggccctccctgtcaccgccctgctgcttccgctggctcttctgctccacgccgctcggcccgaaa |
| CAR 1- | | ttgtgatgacccagtcacccgccactcttagcctttcacccggtgagcgcgcaaccctgtcttgcag |
| Full-nt | | agcctcccaagacatctcaaaataccttaattggtatcaacagaagcccggacaggctcctcgcctt |
| | | ctgatctaccacaccagccggctccattctggaatccctgccaggttcagcggtagcggatctggg |
| | | accgactacaccctcactatcagctcactgcagccagaggacttcgctgtctatttctgtcagcaagg |
| | | gaacaccctgccctacacctttggacagggcaccaagctcgagattaaaggtggaggtggcagcg |
| | | gaggaggtgggtccggcggtggaggaagccaggtccaactccaagaaagcggaccgggtcttg |
| | | tgaagccatcagaaactctttcactgacttgtactgtgagcggagtgtctctccccgattacggggtgt |
| | | cttggatcagacagccaccggggaagggtctggaatggattggagtgatttggggctctgagacta |
| | | cttactactcttcatccctcaagtcacgcgtcaccatctcaaaggacaactctaagaatcaggtgtcac |
| | | tgaaactgtcatctgtgaccgcagccgacaccgccgtgtactattgcgctaagcattactattatggc |
| | | gggagctacgcaatggattactggggacagggtactctggtcaccgtgtccagcaccactacccc |
| | | agcaccgaggccacccaccccggctcctaccatcgcctcccagcctctgtccctgcgtccggagg |
| | | catgtagacccgcagctggtggggccgtgcatacccggggtcttgacttcgcctgcgatatctacat |
| | | ttgggcccctctggctggtacttgcggggtcctgctgctttcactcgtgatcactctttactgtaagcgc |
| | | ggtcggaagaagctgctgtacatctttaagcaacccttcatgaggcctgtgcagactactcaagagg |
| | | aggacggctgttcatgccggttcccagaggaggaggaaggcggctgcgaactgcgcgtgaaatt |
| | | cagccgcagcgcagatgctccagcctacaagcaggggcagaaccagctctacaacgaactcaat |
| | | cttggtcggagagaggagtacgacgtgctggacaagcggagaggacgggacccagaaatgggc |
| | | gggaagccgcgcagaaagaatccccaagagggcctgtacaacgagctccaaaaggataagatg |
| | | gcagaagcctatagcgagattggtatgaaaggggaacgcagaagaggcaaaggccacgacgga |
| | | ctgtaccagggactcagcaccgccaccaaggacacctatgacgctcttcacatgcaggccctgcc |
| | | gcctcgg |
| 104875 | 31 | MALPVTALLLPLALLLHAARPeivmtqspatlslspgeratlscrasgdiskyln |
| CAR 1- | | wyqqkpgqaprlliyhtsrlhsgiparfsgsgsgtdytltisslqpedfavyfcqqgntlpytfgq |
| Full-aa | | gtkleikggggsggggsggggsqvqlqesgpglvkpsetlsltctvsgvslpdygvswirqppg |
| | | kglewigviwgsettyyssslksrvtiskdnsknqvslklssvtaadtavyycakhyyyggsva |
| | | mdywgqgtlvtvsstttpaprpptpaptiasqplslrpeacrpaaggavhtrgldfacdiyiwapl |
| | | agtcgvlllslvitlyckrgrkkllyifkqpfmrpvqttqeedgcscrfpeeeeggcelrvkfsrsad |
| | | apaykqgqnqlynelnlgrreeydvldkrrgrdpemggkprrknpqeglynelqkdkmaea |
| | | yseigmkgerrrgkghdglyqglstatkdtydalhmqalppr |

CAR 2

| | | |
|---|---|---|
| CAR2 | 2 | eivmtqspatlslspgeratlscrasqdiskylnwyqqkpgqaprlliyhtsrlhsgiparfsgsgs |
| scFv | | gtdytltisslqpedfavyfcqqgntlpytfgqgtkleikggggsggggsggggsqvqlqesgpg |
| domain | | lvkpsetlsltctvsgvslpdygvswirqppgkglewigviwgsettyyqsslksrvtiskdnskn |
| | | qvslklssvtaadtavyycakhyyyggsyamdywgqgtlvtvss |
| 103102 | 62 | atggccctccctgtcaccgccctgctgcttccgctggctcttctgctccacgccgctcggcccgaaa |
| CAR2- | | ttgtgatgacccagtcacccgccactcttagcctttcacccggtgagcgcgcaaccctgtcttgcag |
| Soluble | | agcctcccaagacatctcaaaataccttaattggtatcaacagaagcccggacaggctcctcgcctt |
| scFv-nt | | ctgatctaccacaccagccggctccattctggaatccctgccaggttcagcggtagcggatctggg |
| | | accgactacaccctcactatcagctcactgcagccagaggacttcgctgtctatttctgtcagcaagg |
| | | gaacaccctgccctacacctttggacagggcaccaagctcgagattaaaggtggaggtggcagcg |
| | | gaggaggtgggtccggcggtggaggaagccaggtccaactccaagaaagcggaccgggtcttg |
| | | tgaagccatcagaaactctttcactgacttgtactgtgagcggagtgtctctccccgattacggggtgt |
| | | cttggatcagacagccaccggggaagggtctggaatggattggagtgatttggggctctgagacta |
| | | cttactaccaatcatccctcaagtcacgcgtcaccatctcaaaggacaactctaagaatcaggtgtca |
| | | ctgaaactgtcatctgtgaccgcagccgacaccgccgtgtactattgcgctaagcattactattatgg |
| | | cgggagctacgcaatggattactggggacagggtactctggtcaccgtgtccagccaccaccatc |
| | | atcaccatcaccat |
| 103102 | 74 | MALPVTALLLPLALLLHAARPeivmtqspatlslspgeratlscrasqdiskyl |
| CAR2- | | nwyqqkpgqaprlliyhtsrlhsgiparfsgsgsgtdytltisslqpedfavyfcqqgntlpytfgq |
| Soluble | | gtkleikggggsggggsggggsqvqlqesgpglvkpsetlsltctvsgvslpdygvswirqppg |
| scFv-aa | | kglewigviwgsettyyqsslksrvtiskdnsknqvslklssvtaadtavyycakhyyyggsya |
| | | mdywgqgtlvtvsshhhhhhhh |
| 104876 | 86 | atggccctccctgtcaccgccctgctgcttccgctggctcttctgctccacgccgctcggcccgaaa |
| CAR 2- | | ttgtgatgacccagtcacccgccactcttagcctttcacccggtgagcgcgcaaccctgtcttgcag |
| Full-nt | | agcctcccaagacatctcaaaataccttaattggtatcaacagaagcccggacaggctcctcgcctt |
| (also | | ctgatctaccacaccagccggctccattctggaatccctgccaggttcagcggtagcggatctggg |
| referred to | | accgactacaccctcactatcagctcactgcagccagaggacttcgctgtctatttctgtcagcaagg |
| herein as | | gaacaccctgccctacacctttggacagggcaccaagctcgagattaaaggtggaggtggcagcg |
| CTL119 | | gaggaggtgggtccggcggtggaggaagccaggtccaactccaagaaagcggaccgggtcttg |
| nucleotide | | tgaagccatcagaaactctttcactgacttgtactgtgagcggagtgtctctccccgattacggggtgt |
| sequence) | | cttggatcagacagccaccggggaagggtctggaatggattggagtgatttggggctctgagacta |
| | | cttactaccaatcatccctcaagtcacgcgtcaccatctcaaaggacaactctaagaatcaggtgtca |
| | | ctgaaactgtcatctgtgaccgcagccgacaccgccgtgtactattgcgctaagcattactattatgg |
| | | cgggagctacgcaatggattactggggacagggtactctggtcaccgtgtccagcaccactaccc |
| | | cagcaccgaggccacccaccccggctcctaccatcgcctcccagcctctgtccctgcgtccggag |
| | | gcatgtagacccgcagctggtggggccgtgcatacccggggtcttgacttcgcctgcgatatctac |
| | | atttgggcccctctggctggtacttgcggggtcctgctgctttcactcgtgatcactctttactgtaagc |
| | | gcggtcggaagaagctgctgtacatctttaagcaacccttcatgaggcctgtgcagactactcaaga |
| | | ggaggacggctgttcatgccggttcccagaggaggaggaaggcggctgcgaactgcgcgtgaa |
| | | attcagccgcagcgcagatgctccagcctacaagcaggggcagaaccagctctacaacgaactc |
| | | aatcttggtcggagagaggagtacgacgtgctggacaagcggagaggacgggacccagaaatg |
| | | ggcgggaagccgcgcagaaagaatccccaagagggcctgtacaacgagctccaaaaggataag |
| | | atggcagaagcctatagcgagattggtatgaaaggggaacgcagaagaggcaaaggccacgac |
| | | ggactgtaccagggactcagcaccgccaccaaggacacctatgacgctcttcacatgcaggccct |
| | | gccgcctcgg |
| 104876 | 32 | MALPVTALLLPLALLLHAARPeivmtqspatlslspgeratlscrasadiskyln |
| CAR 2- | | wyqqkpgqaprlliyhtsrlhsgiparfsgsgsgtdytltisslqpedfavyfcqqgntlpytfgq |
| Full-aa | | gtkleikggggsggggsggggsqvqlqesgpglvkpsetlsltctvsgvslpdygvswirqppg |
| (also | | kglewigviwgsettyyqsslksrvtiskdnsknqvslklssvtaadtavyycakhyyyggsy |
| referred to | | amdywgqgtlvtvsstttpaprpptpaptiasqplslrpeacrpaaggavhtrgldfacdiyiwa |
| herein as | | plagtcgvlllslvitlyckrgrkkllyifkqpfmrpvqttqeedgcscrfpeeeeggcelrvkfsrs |
| CTL119 | | adapaykqgqnqlynelnlgrreeydvldkrrgrdpemggkprrknpqeglynelqkdkma |
| amino acid | | eayseigmkgerrrgkghdglyqglstatkdtydalhmqalppr |
| sequence) | | |

CAR 3

| | | |
|---|---|---|
| CAR3 | 3 | qvqlqesgpglvkpsetlsltctvsgvslpdygvswirqppgkglewigviwgsettyyssslks |
| scFv | | rvtiskdnsknqvslklssvtaadtavyycakhyyyggsyamdywgqgtlvtvssggggsgg |
| domain | | ggsggggseivmtqspatlslspgeratlscrasqdiskylnwyqqkpgqaprlliyhtsrlhsgi |
| | | parfsgsgsgtdytltisslqpedfavyfcqqgntlpytfgqgtkleik |
| 103104 | 63 | atggctctgcccgtgaccgcactcctcctgccactggctctgctgcttcacgccgctcgcccacaag |
| CAR 3- | | tccagcttcaagaatcagggcctggtctggtgaagccatctgagactctgtccctcacttgcaccgtg |
| Soluble | | agcggagtgtccctcccagactacggagtgagctggattagacagcctcccggaaagggactgg |
| scFv-nt | | agtggatcggagtgatttggggtagcgaaaccacttactattcatcttccctgaagtcacgggtcacc |
| | | atttcaaaggataactcaaagaatcaagtgagcctcaagctctcatcagtcaccgccgctgacaccg |
| | | ccgtgtattactgtgccaagcattactactatggagggtcctacgccatggactactggggccaggg |
| | | aactctggtcactgtgtcatctggtggaggaggtagcggaggaggcgggagcggtggaggtggc |
| | | tccgaaatcgtgatgacccagagccctgcaaccctgtccctttctcccggggaacgggctacccttt |
| | | cttgtcgggcatcacaagatatctcaaaatacctcaattggtatcaacagaagccgggacaggccc |
| | | ctaggcttcttatctaccacacctctcgcctgcatagcgggattcccgcacgctttagcgggtctgga |
| | | agcgggaccgactacactctgaccatctcatctctccagcccgaggacttcgccgtctacttctgcc |
| | | agcagggtaacaccctgccgtacaccttcggccagggcaccaagcttgagatcaaacatcaccac |
| | | catcatcaccatcac |
| 103104 | 75 | MALPVTALLLPLALLLHAARPqvqlqesgpglvkpsetlsltctvsgvslpdy |
| CAR 3- | | gvswirqppgkglewigviwgsettyyssslksrvtiskdnsknqvslklssvtaadtavyycak |
| Soluble | | hyyyggsyamdywgqgtlvtvssggggsggggsggggseivmtqspatlslspgeratlscra |
| scFv-aa | | sqdiskylnwyqqkpgqaprlliyhtsrlhsgiparfsgsgsgtdytltisslqpedfavyfcqqgn |
| | | tlpytfgqgtkleikhhhhhhhh |
| 104877 | 87 | atggctctgcccgtgaccgcactcctcctgccactggctctgctgcttcacgccgctcgcccacaag |
| CAR 3- | | tccagcttcaagaatcagggcctggtctggtgaagccatctgagactctgtccctcacttgcaccgtg |
| Full-nt | | agcggagtgtccctcccagactacggagtgagctggattagacagcctcccggaaagggactgg |
| | | agtggatcggagtgatttggggtagcgaaaccacttactattcatcttccctgaagtcacgggtcacc |
| | | atttcaaaggataactcaaagaatcaagtgagcctcaagctctcatcagtcaccgccgctgacaccg |
| | | ccgtgtattactgtgccaagcattactactatggagggtcctacgccatggactactggggccaggg |
| | | aactctggtcactgtgtcatctggtggaggaggtagcggaggaggcgggagcggtggaggtggc |
| | | tccgaaatcgtgatgacccagagccctgcaaccctgtccctttctcccggggaacgggctacccttt |
| | | cttgtcgggcatcacaagatatctcaaaatacctcaattggtatcaacagaagccgggacaggccc |
| | | ctaggcttcttatctaccacacctctcgcctgcatagcgggattcccgcacgctttagcgggtctgga |
| | | agcgggaccgactacactctgaccatctcatctctccagcccgaggacttcgccgtctacttctgcc |
| | | agcagggtaacaccctgccgtacaccttcggccagggcaccaagcttgagatcaaaaccactact |
| | | cccgctccaaggccacccacccctgccccgaccatcgcctctcagccgctttccctgcgtccgga |
| | | ggcatgtagacccgcagctggtggggccgtgcatacccggggtcttgacttcgcctgcgatatcta |
| | | catttgggcccctctggctggtacttgcggggtcctgctgctttcactcgtgatcactctttactgtaag |
| | | cgcggtcggaagaagctgctgtacatctttaagcaacccttcatgaggcctgtgcagactactcaag |
| | | aggaggacggctgttcatgccggttcccagaggaggaggaaggcggctgcgaactgcgcgtga |
| | | aattcagccgcagcgcagatgctccagcctacaagcaggggcagaaccagctctacaacgaact |
| | | caatcttggtcggagagaggagtacgacgtgctggacaagcggagaggacgggacccagaaat |
| | | gggcgggaagccgcgcagaaagaatccccaagagggcctgtacaacgagctccaaaaggataa |
| | | gatggcagaagcctatagcgagattggtatgaaaggggaacgcagaagaggcaaaggccacga |
| | | cggactgtaccagggactcagcaccgccaccaaggacacctatgacgctcttcacatgcaggccc |
| | | tgccgcctcgg |
| 104877 | 33 | MALPVTALLLPLALLLHAARPqvqlqesgpglvkpsetlsltctvsgvslpdygv |
| CAR 3- | | swirqppgkglewigviwgsettyyssslksrvtiskdnsknqvslklssvtaadtavyycakh |
| Full-aa | | yyyggsyamdywgqgtlvtvssggggsggggsggggseivmtqspatlslspgeratlscra |
| | | sqdiskylnwyqqkpgqaprlliyhtsrlhsgiparfsgsgsgtdytltisslqpedfavyfcqq |
| | | gntlpytfgqgtkleiktttpaprpptpaptiasqplslrpeacrpaaggavhtrgldfacdiyiwa |
| | | plagtcgvlllslvitlyckrgrkkllyifkqpfmrpvqttqeedgcscrfpeeeeggcelrvkfsrs |
| | | adapaykqgqnqlynelnlgrreeydvldkrrgrdpemggkprrknpqeglynelqkdkma |
| | | eayseigmkgerrrgkghdglyqglstatkdtydalhmqalppr |

CAR 4

| | | |
|---|---|---|
| CAR4 | 4 | qvqlqesgpglvkpsetlsltctvsgvslpdygvswirqppgkglewigviwgsettyyqsslks |
| scFv | | rvtiskdnsknqvslklssvtaadtavyycakhyyyggsyamdywgqgtlvtvssggggsgg |
| domain | | ggsggggseivmtqspatlslspgeratlscrasqdiskylnwyqqkpgqaprlliyhtsrlhsgi |
| | | parfsgsgsgtdytltisslqpedfavyfcqqgntlpytfgqgtkleik |
| 103106 | 64 | atggctctgcccgtgaccgcactcctcctgccactggctctgctgcttcacgccgctcgcccacaag |
| CAR4- | | tccagcttcaagaatcagggcctggtctggtgaagccatctgagactctgtccctcacttgcaccgtg |
| Soluble | | agcggagtgtccctcccagactacggagtgagctggattagacagcctcccggaaagggactgg |
| scFv-nt | | agtggatcggagtgatttggggtagcgaaaccacttactatcaatcttccctgaagtcacgggtcac |
| | | catttcaaaggataactcaaagaatcaagtgagcctcaagctctcatcagtcaccgccgctgacacc |
| | | gccgtgtattactgtgccaagcattactactatggagggtcctacgccatggactactggggccagg |
| | | gaactctggtcactgtgtcatctggtggaggaggtagcggaggaggcgggagcggtggaggtgg |
| | | ctccgaaatcgtgatgacccagagccctgcaaccctgtccctttctcccggggaacgggctaccctt |
| | | tcttgtcgggcatcacaagatatctcaaaatacctcaattggtatcaacagaagccgggacaggccc |
| | | ctaggcttcttatctaccacacctctcgcctgcatagcgggattcccgcacgctttagcgggtctgga |
| | | agcgggaccgactacactctgaccatctcatctctccagcccgaggacttcgccgtctacttctgcc |
| | | agcagggtaacaccctgccgtacaccttcggccagggcaccaagcttgagatcaaacatcaccac |
| | | catcatcaccatcac |
| 103106 | 76 | MALPVTALLLPLALLLHAARPqvqlqesgpglvkpsetlsltctvsgvslpdy |
| CAR4- | | gvswirqppgkglewigviwgsettyyqsslksrvtiskdnsknqvslklssvtaadtavyyca |
| Soluble | | khyyyggsyamdywgqgtlvtvssggggsggggsggggseivmtqspatlslspgeratlscr |
| scFv-aa | | asqdiskylnwyqqkpgqaprlliyhtsrlhsgiparfsgsgsgtdytltisslqpedfavyfcqqg |
| | | ntlpytfgqgtkleikhhhhhhhh |
| 104878 | 88 | atggctctgcccgtgaccgcactcctcctgccactggctctgctgcttcacgccgctcgcccacaag |
| CAR 4- | | tccagcttcaagaatcagggcctggtctggtgaagccatctgagactctgtccctcacttgcaccgtg |
| Full-nt | | agcggagtgtccctcccagactacggagtgagctggattagacagcctcccggaaagggactgg |
| | | agtggatcggagtgatttggggtagcgaaaccacttactatcaatcttccctgaagtcacgggtcac |
| | | catttcaaaggataactcaaagaatcaagtgagcctcaagctctcatcagtcaccgccgctgacacc |
| | | gccgtgtattactgtgccaagcattactactatggagggtcctacgccatggactactggggccagg |
| | | gaactctggtcactgtgtcatctggtggaggaggtagcggaggaggcgggagcggtggaggtgg |
| | | ctccgaaatcgtgatgacccagagccctgcaaccctgtccctttctcccggggaacgggctaccctt |
| | | tcttgtcgggcatcacaagatatctcaaaatacctcaattggtatcaacagaagccgggacaggccc |
| | | ctaggcttcttatctaccacacctctcgcctgcatagcgggattcccgcacgctttagcgggtctgga |
| | | agcgggaccgactacactctgaccatctcatctctccagcccgaggacttcgccgtctacttctgcc |
| | | agcagggtaacaccctgccgtacaccttcggccagggcaccaagcttgagatcaaaaccactact |
| | | cccgctccaaggccacccacccctgccccgaccatcgcctctcagccgctttccctgcgtccgga |
| | | ggcatgtagacccgcagctggtggggccgtgcatacccggggtcttgacttcgcctgcgatatcta |
| | | catttgggcccctctggctggtacttgcggggtcctgctgctttcactcgtgatcactctttactgtaag |
| | | cgcggtcggaagaagctgctgtacatctttaagcaacccttcatgaggcctgtgcagactactcaag |
| | | aggaggacggctgttcatgccggttcccagaggaggaggaaggcggctgcgaactgcgcgtga |
| | | aattcagccgcagcgcagatgctccagcctacaagcaggggcagaaccagctctacaacgaact |
| | | caatcttggtcggagagaggagtacgacgtgctggacaagcggagaggacgggacccagaaat |
| | | gggcgggaagccgcgcagaaagaatccccaagagggcctgtacaacgagctccaaaaggataa |
| | | gatggcagaagcctatagcgagattggtatgaaaggggaacgcagaagaggcaaaggccacga |
| | | cggactgtaccagggactcagcaccgccaccaaggacacctatgacgctcttcacatgcaggccc |
| | | tgccgcctcgg |
| 104878 | 34 | MALPVTALLLPLALLLHAARPqvqlqesgpglvkpsetlsltctvsgvslpdygv |
| CAR 4- | | swirqppgkglewigviwgsettyyqsslksrvtiskdnsknqvslklssvtaadtavyycakh |
| Full-aa | | yyyggsvamdvwgqgtlvtvssggggsggggsggggseivmtqspatlslspgeratlscra |
| | | sqdiskylnwyqqkpgqaprlliyhtsrlhsgiparfsgsgsgtdvtltisslqpedfavvfcqq |
| | | gntlpytfgqgtkleiktttpaprpptpaptiasqplslrpeacrpaaggavhtrgldfacdiyiwa |
| | | plagtcgvlllslvitlyckrgrkkllyifkqpfmrpvqttqeedgcscrfpeeeeggcelrvkfsrs |
| | | adapaykqgqnqlynelnlgrreeydvldkrrgrdpemggkprrknpqeglynelqkdkma |
| | | eayseigmkgerrrgkghdglyqglstatkdtydalhmqalppr |

CAR 5

| | | |
|---|---|---|
| CAR5 | 5 | eivmtqspatlslspgeratlscrasqdiskylnwyqqkpgqaprlliyhtsrlhsgiparfsgsgs |
| scFv | | gtdytltisslqpedfavyfcqqgntlpytfgqgtkleikggggsggggsggggsggggsqvqlq |
| domain | | esgpglvkpsetlsltctvsgvslpdygvswirqppgkglewigviwgsettyyssslksrvtisk |
| | | dnsknqvslklssvtaadtavyycakhyyyggsyamdywgqgtlvtvss |
| 99789 | 65 | atggccctcccagtgaccgctctgctgctgcctctcgcacttcttctccatgccgctcggcctgagat |
| CAR5- | | cgtcatgacccaaagccccgctaccctgtccctgtcacccggcgagagggcaaccctttcatgcag |
| Soluble | | ggccagccaggacatttctaagtacctcaactggtatcagcagaagccagggcaggctcctcgcct |
| scFv-nt | | gctgatctaccacaccagccgcctccacagcggtatccccgccagattttccgggagcgggtctgg |
| | | aaccgactacaccctcaccatctcttctctgcagcccgaggatttcgccgtctatttctgccagcagg |
| | | ggaatactctgccgtacaccttcggtcaaggtaccaagctggaaatcaagggaggcggaggatca |
| | | ggcggtggcggaagcggaggaggtggctccggaggaggaggttcccaagtgcagcttcaagaa |
| | | tcaggacccggacttgtgaagccatcagaaaccctctccctgacttgtaccgtgtccggtgtgagcc |
| | | tccccgactacggagtctcttggattcgccagcctccggggaagggtcttgaatggattggggtgat |
| | | ttggggatcagagactacttactactcttcatcacttaagtcacgggtcaccatcagcaaagataata |
| | | gcaagaaccaagtgtcacttaagctgtcatctgtgaccgccgctgacaccgccgtgtactattgtgc |
| | | caaacattactattacggagggtcttatgctatggactactggggacaggggaccctggtgactgtct |
| | | ctagccatcaccatcaccaccatcatcac |
| 99789 | 77 | MALPVTALLLPLALLLHAARPeivmtqspatlslspgeratlscrasqdiskyl |
| CAR5- | | nwyqqkpgqaprlliyhtsrlhsgiparfsgsgsgtdytltisslqpedfavyfcqqgntlpytfgq |
| Soluble | | gtkleikggggsggggsggggsggggsqvqlqesgpglvkpsetlsltctvsgvslpdygvswi |
| scFv-aa | | rqppgkglewigviwgsettyyssslksrvtiskdnsknqvslklssvtaadtavyycakhyyy |
| | | ggsyamdywgqgtlvtvsshhhhhhhh |
| 104879 | 89 | atggccctccctgtcaccgccctgctgcttccgctggctcttctgctccacgccgctcggcccgaaa |
| CAR 5- | | ttgtgatgacccagtcacccgccactcttagcctttcacccggtgagcgcgcaaccctgtcttgcag |
| Full-nt | | agcctcccaagacatctcaaaataccttaattggtatcaacagaagcccggacaggctcctcgcctt |
| | | ctgatctaccacaccagccggctccattctggaatccctgccaggttcagcggtagcggatctggg |
| | | accgactacaccctcactatcagctcactgcagccagaggacttcgctgtctatttctgtcagcaagg |
| | | gaacaccctgccctacacctttggacagggcaccaagctcgagattaaaggtggaggtggcagcg |
| | | gaggaggtgggtccggcggtggaggaagcggcggaggcgggagccaggtccaactccaaga |
| | | aagcggaccgggtcttgtgaagccatcagaaactctttcactgacttgtactgtgagcggagtgtctc |
| | | tccccgattacggggtgtcttggatcagacagccaccggggaagggtctggaatggattggagtg |
| | | atttggggctctgagactacttactactcttcatccctcaagtcacgcgtcaccatctcaaaggacaac |
| | | tctaagaatcaggtgtcactgaaactgtcatctgtgaccgcagccgacaccgccgtgtactattgcg |
| | | ctaagcattactattatggcgggagctacgcaatggattactggggacagggtactctggtcaccgt |
| | | gtccagcaccactaccccagcaccgaggccacccaccccggctcctaccatcgcctcccagcctc |
| | | tgtccctgcgtccggaggcatgtagacccgcagctggtggggccgtgcatacccggggtcttgact |
| | | tcgcctgcgatatctacatttgggcccctctggctggtacttgcggggtcctgctgctttcactcgtgat |
| | | cactctttactgtaagcgcggtcggaagaagctgctgtacatctttaagcaacccttcatgaggcctg |
| | | tgcagactactcaagaggaggacggctgttcatgccggttcccagaggaggaggaaggcggctg |
| | | cgaactgcgcgtgaaattcagccgcagcgcagatgctccagcctacaagcaggggcagaaccag |
| | | ctctacaacgaactcaatcttggtcggagagaggagtacgacgtgctggacaagcggagaggac |
| | | gggacccagaaatgggcgggaagccgcgcagaaagaatccccaagagggcctgtacaacgag |
| | | ctccaaaaggataagatggcagaagcctatagcgagattggtatgaaaggggaacgcagaagag |
| | | gcaaaggccacgacggactgtaccagggactcagcaccgccaccaaggacacctatgacgctct |
| | | tcacatgcaggccctgccgcctcgg |
| 104879 | 35 | MALPVTALLLPLALLLHAARPeivmtqspatlslspgeratlscrasqdiskyln |
| CAR 5- | | wyqqkpgqaprlliyhtsrlhsgiparfsgsgsgtdytltisslqpedfavyfcqqgntlpytfgq |
| Full-aa | | gtkleikggggsggggsggggsggggsqvqlqesgpglvkpsetlsltctvsgvslpdygvswi |
| | | rqppgkglewigviwgsettyyssslksrvtiskdnsknqvslklssvtaadtavyycakhyyy |
| | | ggsyamdywgqgtlvtvsstttpaprpptpaptiasqplslrpeacrpaaggavhtrgldfacdi |
| | | yiwaplagtcgvlllslvitlyckrgrkkllyifkqpfmrpvqttqeedgcscrfpeeeeggcelrv |
| | | kfsrsadapaykqgqnqlynelnlgrreeydvldkrrgrdpemggkprrknpqeglynelqkd |
| | | kmaeayseigmkgerrrgkghdglyqglstatkdtydalhmqalppr |

CAR 6

| | | |
|---|---|---|
| CAR6 | 6 | eivmtqspatlslspgeratlscrasqdiskylnwyqqkpgqaprlliyhtsrlhsgiparfsgsgs |
| scFv | | gtdytltisslqpedfavyfcqqgntlpytfgqgtkleikggggsggggsggggsggggsqvqlq |
| domain | | esgpglvkpsetlsltctvsgvslpdygvswirqppgkglewigviwgsettyyqsslksrvtisk |
| | | dnsknqvslklssvtaadtavyycakhyyyggsyamdywgqgtlvtvss |
| 99790 | 66 | atggccctcccagtgaccgctctgctgctgcctctcgcacttcttctccatgccgctcggcctgagat |
| CAR6- | | cgtcatgacccaaagccccgctaccctgtccctgtcacccggcgagagggcaaccctttcatgcag |
| Soluble | | ggccagccaggacatttctaagtacctcaactggtatcagcagaagccagggcaggctcctcgcct |
| scFv-nt | | gctgatctaccacaccagccgcctccacagcggtatccccgccagattttccgggagcgggtctgg |
| | | aaccgactacaccctcaccatctcttctctgcagcccgaggatttcgccgtctatttctgccagcagg |
| | | ggaatactctgccgtacaccttcggtcaaggtaccaagctggaaatcaagggaggcggaggatca |
| | | ggcggtggcggaagcggaggaggtggctccggaggaggaggttcccaagtgcagcttcaagaa |
| | | tcaggacccggacttgtgaagccatcagaaaccctctccctgacttgtaccgtgtccggtgtgagcc |
| | | tccccgactacggagtctcttggattcgccagcctccggggaagggtcttgaatggattggggtgat |
| | | ttggggatcagagactacttactaccagtcatcacttaagtcacgggtcaccatcagcaaagataata |
| | | gcaagaaccaagtgtcacttaagctgtcatctgtgaccgccgctgacaccgccgtgtactattgtgc |
| | | caaacattactattacggagggtcttatgctatggactactggggacaggggaccctggtgactgtct |
| | | ctagccatcaccatcaccaccatcatcac |
| 99790 | 78 | MALPVTALLLPLALLLHAARPeivmtqspatlslspgeratlscrasqdiskyl |
| CAR6- | | nwyqqkpgqaprlliyhtsrlhsgiparfsgsgsgtdytltisslqpedfavyfcqqgntlpytfgq |
| Soluble | | gtkleikggggsggggsggggsggggsqvqlqesgpglvkpsetlsltctvsgvslpdygvswi |
| scFv-aa | | rqppgkglewigviwgsettyyqsslksrvtiskdnsknqvslklssvtaadtavyycakhyyy |
| | | ggsyamdywgqgtlvtvsshhhhhhhh |
| 104880 | 90 | atggccctccctgtcaccgccctgctgcttccgctggctcttctgctccacgccgctcggcccgaaa |
| CAR6- | | ttgtgatgacccagtcacccgccactcttagcctttcacccggtgagcgcgcaaccctgtcttgcag |
| Full-nt | | agcctcccaagacatctcaaaataccttaattggtatcaacagaagcccggacaggctcctcgcctt |
| | | ctgatctaccacaccagccggctccattctggaatccctgccaggttcagcggtagcggatctggg |
| | | accgactacaccctcactatcagctcactgcagccagaggacttcgctgtctatttctgtcagcaagg |
| | | gaacaccctgccctacacctttggacagggcaccaagctcgagattaaaggtggaggtggcagcg |
| | | gaggaggtgggtccggcggtggaggaagcggaggcggagggagccaggtccaactccaaga |
| | | aagcggaccgggtcttgtgaagccatcagaaactctttcactgacttgtactgtgagcggagtgtctc |
| | | tccccgattacggggtgtcttggatcagacagccaccggggaagggtctggaatggattggagtg |
| | | atttggggctctgagactacttactaccaatcatccctcaagtcacgcgtcaccatctcaaaggacaa |
| | | ctctaagaatcaggtgtcactgaaactgtcatctgtgaccgcagccgacaccgccgtgtactattgc |
| | | gctaagcattactattatggcgggagctacgcaatggattactggggacagggtactctggtcaccg |
| | | tgtccagcaccactaccccagcaccgaggccacccaccccggctcctaccatcgcctcccagcct |
| | | ctgtccctgcgtccggaggcatgtagacccgcagctggtggggccgtgcatacccggggtcttga |
| | | cttcgcctgcgatatctacatttgggcccctctggctggtacttgcggggtcctgctgctttcactcgtg |
| | | atcactctttactgtaagcgcggtcggaagaagctgctgtacatctttaagcaacccttcatgaggcct |
| | | gtgcagactactcaagaggaggacggctgttcatgccggttcccagaggaggaggaaggcggct |
| | | gcgaactgcgcgtgaaattcagccgcagcgcagatgctccagcctacaagcaggggcagaacca |
| | | gctctacaacgaactcaatcttggtcggagagaggagtacgacgtgctggacaagcggagagga |
| | | cgggacccagaaatgggcgggaagccgcgcagaaagaatccccaagagggcctgtacaacga |
| | | gctccaaaaggataagatggcagaagcctatagcgagattggtatgaaaggggaacgcagaaga |
| | | ggcaaaggccacgacggactgtaccagggactcagcaccgccaccaaggacacctatgacgct |
| | | cttcacatgcaggccctgccgcctcgg |
| 104880 | 36 | MALPVTALLLPLALLLHAARPeivmtqspatlslspgeratlscrasqdiskyln |
| CAR6- | | wyqqkpgqaprlliyhtsrlhsgiparfsgsgsgtdytltisslqpedfavyfcqqgntlpytfgq |
| Full-aa | | gtkleikggggsggggsggggsggggsqvqlqesgpglvkpsetlsltctvsgvslpdygvswi |
| | | rqppgkglewigviwgsettyygsslksrvtiskdnsknqvslklssvtaadtavyycakhyyy |
| | | ggsyamdywgqgtlvtvsstttpaprpptpaptiasqplslrpeacrpaaggavhtrgldfacdi |
| | | yiwaplagtcgvlllslvitlyckrgrkkllyifkqpfmrpvqttqeedgcscrfpeeeeggcelrv |
| | | kfsrsadapaykqgqnqlynelnlgrreeydvldkrrgrdpemggkprrknpqeglynelqkd |
| | | kmaeayseigmkgerrrgkghdglyqglstatkdtydalhmqalppr |

CAR 7

| | | |
|---|---|---|
| CAR7 | 7 | qvqlqesgpglvkpsetlsltctvsgvslpdygvswirqppgkglewigviwgsettyyssslks |
| scFv | | rvtiskdnsknqvslklssvtaadtavyycakhyyyggsyamdywgqgtlvtvssggggsgg |
| domain | | ggsggggsggggseivmtqspatlslspgeratlscrasqdiskylnwyqqkpgqaprlliyhts |
| | | rlhsgiparfsgsgsgtdytltisslqpedfavyfcqqgntlpytfgqgtkleik |
| 100796 | 67 | atggcactgcctgtcactgccctcctgctgcctctggccctccttctgcatgccgccaggccccaag |
| CAR7- | | tccagctgcaagagtcaggacccggactggtgaagccgtctgagactctctcactgacttgtaccgt |
| Soluble | | cagcggcgtgtccctccccgactacggagtgtcatggatccgccaacctcccgggaaagggcttg |
| scFv-nt | | aatggattggtgtcatctggggttctgaaaccacctactactcatcttccctgaagtccagggtgacc |
| | | atcagcaaggataattccaagaaccaggtcagccttaagctgtcatctgtgaccgctgctgacaccg |
| | | ccgtgtattactgcgccaagcactactattacggaggaagctacgctatggactattggggacagg |
| | | gcactctcgtgactgtgagcagcggcggtggagggtctggaggtggaggatccggtggtggtgg |
| | | gtcaggcggaggagggagcgagattgtgatgactcagtcaccagccaccctttctctttcacccgg |
| | | cgagagagcaaccctgagctgtagagccagccaggacatttctaagtacctcaactggtatcagca |
| | | aaaaccggggcaggcccctcgcctcctgatctaccatacctcacgccttcactctggtatccccgct |
| | | cggtttagcggatcaggatctggtaccgactacactctgaccatttccagcctgcagccagaagattt |
| | | cgcagtgtatttctgccagcagggcaatacccttccttacaccttcggtcagggaaccaagctcgaa |
| | | atcaagcaccatcaccatcatcaccaccat |
| 100796 | 79 | MALPVTALLLPLALLLHAARPqvqlqesgpglvkpsetlsltctvsgvslpdy |
| CAR7- | | gvswirqppgkglewigviwgsettyyssslksrvtiskdnsknqvslklssvtaadtavyycak |
| Soluble | | hyyyggsyamdywgqgtlvtvssggggsggggsggggsggggseivmtqspatlslspger |
| scFv-aa | | atlscrasqdiskylnwyqqkpgqaprlliyhtsrlhsgiparfsgsgsgtdytltisslqpedfavy |
| | | fcqqgntlpytfgqgtkleikhhhhhhhh |
| 104881 | 91 | atggctctgcccgtgaccgcactcctcctgccactggctctgctgcttcacgccgctcgcccacaag |
| CAR 7 | | tccagcttcaagaatcagggcctggtctggtgaagccatctgagactctgtccctcacttgcaccgtg |
| Full-nt | | agcggagtgtccctcccagactacggagtgagctggattagacagcctcccggaaagggactgg |
| | | agtggatcggagtgatttggggtagcgaaaccacttactattcatcttccctgaagtcacgggtcacc |
| | | atttcaaaggataactcaaagaatcaagtgagcctcaagctctcatcagtcaccgccgctgacaccg |
| | | ccgtgtattactgtgccaagcattactactatggagggtcctacgccatggactactggggccaggg |
| | | aactctggtcactgtgtcatctggtggaggaggtagcggaggaggcgggagcggtggaggtggc |
| | | tccggaggtggcggaagcgaaatcgtgatgacccagagccctgcaaccctgtccctttctcccgg |
| | | ggaacgggctaccctttcttgtcgggcatcacaagatatctcaaaatacctcaattggtatcaacaga |
| | | agccgggacaggcccctaggcttcttatctaccacacctctcgcctgcatagcgggattcccgcac |
| | | gctttagcgggtctggaagcgggaccgactacactctgaccatctcatctctccagcccgaggactt |
| | | cgccgtctacttctgccagcagggtaacaccctgccgtacaccttcggccagggcaccaagcttga |
| | | gatcaaaaccactactcccgctccaaggccacccacccctgccccgaccatcgcctctcagccgct |
| | | ttccctgcgtccggaggcatgtagacccgcagctggtggggccgtgcatacccggggtcttgactt |
| | | cgcctgcgatatctacatttgggcccctctggctggtacttgcggggtcctgctgctttcactcgtgat |
| | | cactctttactgtaagcgcggtcggaagaagctgctgtacatctttaagcaacccttcatgaggcctg |
| | | tgcagactactcaagaggaggacggctgttcatgccggttcccagaggaggaggaaggcggctg |
| | | cgaactgcgcgtgaaattcagccgcagcgcagatgctccagcctacaagcaggggcagaaccag |
| | | ctctacaacgaactcaatcttggtcggagagaggagtacgacgtgctggacaagcggagaggac |
| | | gggacccagaaatgggcgggaagccgcgcagaaagaatccccaagagggcctgtacaacgag |
| | | ctccaaaaggataagatggcagaagcctatagcgagattggtatgaaaggggaacgcagaagag |
| | | gcaaaggccacgacggactgtaccagggactcagcaccgccaccaaggacacctatgacgctct |
| | | tcacatgcaggccctgccgcctcgg |
| 104881 | 37 | MALPVTALLLPLALLLHAARPqvqlqesgpglvkpsetlsltctvsgvslpdygv |
| CAR 7 | | swirqppgkglewigviwgsettyyssslksrvtiskdnsknqvslklssvtaadtavyycakh |
| Full-aa | | yyyggsyamdywgqgtlvtvssggggsggggsggggsggggseivmtqspatlslspgera |
| | | tlscrasqdiskylnwyqqkpgqaprlliyhtsrlhsgiparfsgsgsgtdytltisslqpedfav |
| | | yfcqqgntlpytfgqgtkleiktttpaprpptpaptiasqplslrpeacrpaaggavhtrgldfacd |
| | | iyiwaplagtcgvlllslvitlyckrgrkkllyifkqpfmrpvqttqeedgcscrfpeeeeggcelr |
| | | vkfsrsadapaykqgqnqlynelnlgrreeydvldkrrgrdpemggkprrknpqeglynelqk |
| | | dkmaeayseigmkgerrrgkghdglyqglstatkdtydalhmqalppr |

CAR 8

| | | |
|---|---|---|
| CAR8 | 8 | qvqlqesgpglvkpsetlsltctvsgvslpdygvswirqppgkglewigviwgsettyyqsslks |
| scFv | | rvtiskdnsknqvslklssvtaadtavyycakhyyyggsyamdywgqgtlvtvssggggsgg |
| domain | | ggsggggsggggseivmtqspatlslspgeratlscrasqdiskylnwyqqkpgqaprlliyhts |
| | | rlhsgiparfsgsgsgtdytltisslqpedfavyfcqqgntlpytfgqgtkleik |
| 100798 | 68 | atggcactgcctgtcactgccctcctgctgcctctggccctccttctgcatgccgccaggccccaag |
| CAR8- | | tccagctgcaagagtcaggacccggactggtgaagccgtctgagactctctcactgacttgtaccgt |
| Soluble | | cagcggcgtgtccctccccgactacggagtgtcatggatccgccaacctcccgggaaagggcttg |
| scFv-nt | | aatggattggtgtcatctggggttctgaaaccacctactaccagtcttccctgaagtccagggtgacc |
| | | atcagcaaggataattccaagaaccaggtcagccttaagctgtcatctgtgaccgctgctgacaccg |
| | | ccgtgtattactgcgccaagcactactattacggaggaagctacgctatggactattggggacagg |
| | | gcactctcgtgactgtgagcagcggcggtggagggtctggaggtggaggatccggtggtggtgg |
| | | gtcaggcggaggagggagcgagattgtgatgactcagtcaccagccaccctttctctttcacccgg |
| | | cgagagagcaaccctgagctgtagagccagccaggacatttctaagtacctcaactggtatcagca |
| | | aaaaccggggcaggcccctcgcctcctgatctaccatacctcacgccttcactctggtatccccgct |
| | | cggtttagcggatcaggatctggtaccgactacactctgaccatttccagcctgcagccagaagattt |
| | | cgcagtgtatttctgccagcagggcaatacccttccttacaccttcggtcagggaaccaagctcgaa |
| | | atcaagcaccatcaccatcatcatcaccac |
| 100798 | 80 | MALPVTALLLPLALLLHAARPqvqlqesgpglvkpsetlsltctvsgvslpdy |
| CAR8- | | gvswirqppgkglewigviwgsettyyqsslksrvtiskdnsknqvslklssvtaadtavyyca |
| Soluble | | khyyyggsyamdywgqgtlvtvssggggsggggsggggsggggseivmtqspatlslspge |
| scFv-aa | | ratlscrasqdiskylnwyqqkpgqaprlliyhtsrlhsgiparfsgsgsgtdytltisslqpedfav |
| | | yfcqqgntlpytfgqgtkleikhhhhhhhh |
| 104882 | 92 | atggctctgcccgtgaccgcactcctcctgccactggctctgctgcttcacgccgctcgcccacaag |
| CAR 8- | | tccagcttcaagaatcagggcctggtctggtgaagccatctgagactctgtccctcacttgcaccgtg |
| Full-nt | | agcggagtgtccctcccagactacggagtgagctggattagacagcctcccggaaagggactgg |
| | | agtggatcggagtgatttggggtagcgaaaccacttactatcaatcttccctgaagtcacgggtcac |
| | | catttcaaaggataactcaaagaatcaagtgagcctcaagctctcatcagtcaccgccgctgacacc |
| | | gccgtgtattactgtgccaagcattactactatggagggtcctacgccatggactactggggccagg |
| | | gaactctggtcactgtgtcatctggtggaggaggtagcggaggaggcgggagcggtggaggtgg |
| | | ctccggaggcggtgggtcagaaatcgtgatgacccagagccctgcaaccctgtccctttctcccgg |
| | | ggaacgggctaccctttcttgtcgggcatcacaagatatctcaaaatacctcaattggtatcaacaga |
| | | agccgggacaggcccctaggcttcttatctaccacacctctcgcctgcatagcgggattcccgcac |
| | | gctttagcgggtctggaagcgggaccgactacactctgaccatctcatctctccagcccgaggactt |
| | | cgccgtctacttctgccagcagggtaacaccctgccgtacaccttcggccagggcaccaagcttga |
| | | gatcaaaaccactactcccgctccaaggccacccacccctgccccgaccatcgcctctcagccgct |
| | | ttccctgcgtccggaggcatgtagacccgcagctggtggggccgtgcatacccggggtcttgactt |
| | | cgcctgcgatatctacatttgggcccctctggctggtacttgcggggtcctgctgctttcactcgtgat |
| | | cactctttactgtaagcgcggtcggaagaagctgctgtacatctttaagcaacccttcatgaggcctg |
| | | tgcagactactcaagaggaggacggctgttcatgccggttcccagaggaggaggaaggcggctg |
| | | cgaactgcgcgtgaaattcagccgcagcgcagatgctccagcctacaagcaggggcagaaccag |
| | | ctctacaacgaactcaatcttggtcggagagaggagtacgacgtgctggacaagcggagaggac |
| | | gggacccagaaatgggcgggaagccgcgcagaaagaatccccaagagggcctgtacaacgag |
| | | ctccaaaaggataagatggcagaagcctatagcgagattggtatgaaaggggaacgcagaagag |
| | | gcaaaggccacgacggactgtaccagggactcagcaccgccaccaaggacacctatgacgctct |
| | | tcacatgcaggccctgccgcctcgg |
| 104882 | 38 | MALPVTALLLPLALLLHAARPqvqlqesgpglvkpsetlsltctvsgvslpdygv |
| CAR 8- | | swirqppgkglewigviwgsettyyqsslksrvtiskdnsknqvslklssvtaadtavyycakh |
| Full-aa | | yyyggsyamdywgqgtlvtvssggggsggggsggggsggggseivmtqspatlslspgera |
| | | tlscrasqdiskylnwyqqkpgqaprlliyhtsrlhsgiparfsgsgsgtdytltisslqpedfav |
| | | yfcqqgntlpytfgqgtkleiktttpaprpptpaptiasqplslrpeacrpaaggavhtrgldfacd |
| | | iyiwaplagtcgvlllslvitlyckrgrkkllyifkqpfmrpvqttqeedgcscrfpeeeeggcelr |
| | | vkfsrsadapaykqgqnqlynelnlgrreeydvldkrrgrdpemggkprrknpqeglynelqk |
| | | dkmaeayseigmkgerrrgkghdglyqglstatkdtydalhmqalppr |

CAR 9

| | | |
|---|---|---|
| CAR9 | 9 | eivmtqspatlslspgeratlscrasqdiskylnwyqqkpgqaprlliyhtsrlhsgiparfsgsgs |
| scFv | | gtdytltisslqpedfavyfcqqgntlpytfgqgtkleikggggsggggsggggsggggsqvqlq |
| domain | | esgpglvkpsetlsltctvsgvslpdygvswirqppgkglewigviwgsettyynsslksrvtisk |
| | | dnsknqvslklssvtaadtavyycakhyyyggsyamdywgqgtlvtvss |
| 99789 | 69 | atggccctcccagtgaccgctctgctgctgcctctcgcacttcttctccatgccgctcggcctgagat |
| CAR9- | | cgtcatgacccaaagccccgctaccctgtccctgtcacccggcgagagggcaaccctttcatgcag |
| Soluble | | ggccagccaggacatttctaagtacctcaactggtatcagcagaagccagggcaggctcctcgcct |
| scFv-nt | | gctgatctaccacaccagccgcctccacagcggtatccccgccagattttccgggagcgggtctgg |
| | | aaccgactacaccctcaccatctcttctctgcagcccgaggatttcgccgtctatttctgccagcagg |
| | | ggaatactctgccgtacaccttcggtcaaggtaccaagctggaaatcaagggaggcggaggatca |
| | | ggcggtggcggaagcggaggaggtggctccggaggaggaggttcccaagtgcagcttcaagaa |
| | | tcaggacccggacttgtgaagccatcagaaaccctctccctgacttgtaccgtgtccggtgtgagcc |
| | | tccccgactacggagtctcttggattcgccagcctccggggaagggtcttgaatggattggggtgat |
| | | ttggggatcagagactacttactacaattcatcacttaagtcacgggtcaccatcagcaaagataata |
| | | gcaagaaccaagtgtcacttaagctgtcatctgtgaccgccgctgacaccgccgtgtactattgtgc |
| | | caaacattactattacggagggtcttatgctatggactactggggacaggggaccctggtgactgtct |
| | | ctagccatcaccatcaccaccatcatcac |
| 99789 | 81 | MALPVTALLLPLALLLHAARPeivmtqspatlslspgeratlscrasqdiskyl |
| CAR9- | | nwyqqkpgqaprlliyhtsrlhsgiparfsgsgsgtdytltisslqpedfavyfcqqgntlpytfgq |
| Soluble | | gtkleikggggsggggsggggsggggsqvqlqesgpglvkpsetlsltctvsgvslpdygvswi |
| scFv-aa | | rqppgkglewigviwgsettyynsslksrvtiskdnsknqvslklssvtaadtavyycakhyyy |
| | | ggsyamdywgqgtivtvsshhhhhhhh |
| 105974 | 93 | atggccctccctgtcaccgccctgctgcttccgctggctcttctgctccacgccgctcggcccgaaa |
| CAR 9- | | ttgtgatgacccagtcacccgccactcttagcctttcacccggtgagcgcgcaaccctgtcttgcag |
| Full-nt | | agcctcccaagacatctcaaaataccttaattggtatcaacagaagcccggacaggctcctcgcctt |
| | | ctgatctaccacaccagccggctccattctggaatccctgccaggttcagcggtagcggatctggg |
| | | accgactacaccctcactatcagctcactgcagccagaggacttcgctgtctatttctgtcagcaagg |
| | | gaacaccctgccctacacctttggacagggcaccaagctcgagattaaaggtggaggtggcagcg |
| | | gaggaggtgggtccggcggtggaggaagcggaggcggtgggagccaggtccaactccaagaa |
| | | agcggaccgggtcttgtgaagccatcagaaactctttcactgacttgtactgtgagcggagtgtctct |
| | | ccccgattacggggtgtcttggatcagacagccaccggggaagggtctggaatggattggagtga |
| | | tttggggctctgagactacttactacaactcatccctcaagtcacgcgtcaccatctcaaaggacaac |
| | | tctaagaatcaggtgtcactgaaactgtcatctgtgaccgcagccgacaccgccgtgtactattgcg |
| | | ctaagcattactattatggcgggagctacgcaatggattactggggacagggtactctggtcaccgt |
| | | gtccagcaccactaccccagcaccgaggccacccaccccggctcctaccatcgcctcccagcctc |
| | | tgtccctgcgtccggaggcatgtagacccgcagctggtggggccgtgcatacccggggtcttgact |
| | | tcgcctgcgatatctacatttgggcccctctggctggtacttgcggggtcctgctgctttcactcgtgat |
| | | cactctttactgtaagcgcggtcggaagaagctgctgtacatctttaagcaacccttcatgaggcctg |
| | | tgcagactactcaagaggaggacggctgttcatgccggttcccagaggaggaggaaggcggctg |
| | | cgaactgcgcgtgaaattcagccgcagcgcagatgctccagcctacaagcaggggcagaaccag |
| | | ctctacaacgaactcaatcttggtcggagagaggagtacgacgtgctggacaagcggagaggac |
| | | gggacccagaaatgggcgggaagccgcgcagaaagaatccccaagagggcctgtacaacgag |
| | | ctccaaaaggataagatggcagaagcctatagcgagattggtatgaaaggggaacgcagaagag |
| | | gcaaaggccacgacggactgtaccagggactcagcaccgccaccaaggacacctatgacgctct |
| | | tcacatgcaggccctgccgcctcgg |
| 105974 | 39 | MALPVTALLLPLALLLHAARPeivmtqspatlslspgeratlscrasadiskyln |
| CAR 9- | | wyqqkpgqaprlliyhtsrlhsgiparfsgsgsgtdvtltisslqpedfavyfcqqgntlpytfgq |
| Full-aa | | gtkleikggggsggggsggggsggggsqvqlqesgpglvkpsetlsltctvsgvslpdygvswi |
| | | rqppgkglewigviwgsettyynsslksrvtiskdnsknqvslklssvtaadtavyycakhyyy |
| | | ggsyamdywgqgtlvtvsstttpaprpptpaptiasqplslrpeacrpaaggavhtrgldfacdi |
| | | yiwaplagtcgvlllslvitlyckrgrkkllyifkqpfmrpvqttqeedgcscrfpeeeeggcelrv |
| | | kfsrsadapaykqgqnqlynelnlgrreeydvldkrrgrdpemggkprrknpqeglynelqkd |
| | | kmaeayseigmkgerrrgkghdglyqglstatkdtydalhmqalppr |

CAR10

| | | |
|---|---|---|
| CAR10 | 10 | qvqlqesgpglvkpsetlsltctvsgvslpdygvswirqppgkglewigviwgsettyynsslks |
| scFv | | rvtiskdnsknqvslklssvtaadtavyycakhyyyggsyamdywgqgtlvtvssggggsgg |
| domain | | ggsggggsggggseivmtqspatlslspgeratlscrasqdiskylnwyqqkpgqaprlliyhts |
| | | rlhsgiparfsgsgsgtdytltisslqpedfavyfcqqgntlpytfgqgtkleik |
| 100796 | 70 | atggcactgcctgtcactgccctcctgctgcctctggccctccttctgcatgccgccaggccccaag |
| CAR10- | | tccagctgcaagagtcaggacccggactggtgaagccgtctgagactctctcactgacttgtaccgt |
| Soluble | | cagcggcgtgtccctccccgactacggagtgtcatggatccgccaacctcccgggaaagggcttg |
| scFv-nt | | aatggattggtgtcatctggggttctgaaaccacctactacaactcttccctgaagtccagggtgacc |
| | | atcagcaaggataattccaagaaccaggtcagccttaagctgtcatctgtgaccgctgctgacaccg |
| | | ccgtgtattactgcgccaagcactactattacggaggaagctacgctatggactattggggacagg |
| | | gcactctcgtgactgtgagcagcggcggtggagggtctggaggtggaggatccggtggtggtgg |
| | | gtcaggcggaggagggagcgagattgtgatgactcagtcaccagccaccctttctctttcacccgg |
| | | cgagagagcaaccctgagctgtagagccagccaggacatttctaagtacctcaactggtatcagca |
| | | aaaaccggggcaggcccctcgcctcctgatctaccatacctcacgccttcactctggtatccccgct |
| | | cggtttagcggatcaggatctggtaccgactacactctgaccatttccagcctgcagccagaagattt |
| | | cgcagtgtatttctgccagcagggcaatacccttccttacaccttcggtcagggaaccaagctcgaa |
| | | atcaagcaccatcaccatcatcaccaccat |
| 100796 | 82 | MALPVTALLLPLALLLHAARPqvqlqesgpglvkpsetlsltctvsgvslpdy |
| CAR10- | | gvswirqppgkglewigviwgsettyynsslksrvtiskdnsknqvslklssvtaadtavyyca |
| Soluble | | khyyyggsyamdywgqgtlvtvssggggsggggsggggsggggseivmtqspatlslspge |
| scFv-aa | | ratlscrasqdiskylnwyqqkpgqaprlliyhtsrlhsgiparfsgsgsgtdytltisslqpedfav |
| | | yfcqqgntlpytfgqgtkleikhhhhhhhh |
| 105975 | 94 | atggccctccctgtcaccgccctgctgcttccgctggctcttctgctccacgccgctcggcccgaaa |
| CAR 10- | | ttgtgatgacccagtcacccgccactcttagcctttcacccggtgagcgcgcaaccctgtcttgcag |
| Full-nt | | agcctcccaagacatctcaaaataccttaattggtatcaacagaagcccggacaggctcctcgcctt |
| | | ctgatctaccacaccagccggctccattctggaatccctgccaggttcagcggtagcggatctggg |
| | | accgactacaccctcactatcagctcactgcagccagaggacttcgctgtctatttctgtcagcaagg |
| | | gaacaccctgccctacacctttggacagggcaccaagctcgagattaaaggtggaggtggcagcg |
| | | gaggaggtgggtccggcggtggaggaagcggaggcggtgggagccaggtccaactccaagaa |
| | | agcggaccgggtcttgtgaagccatcagaaactctttcactgacttgtactgtgagcggagtgtctct |
| | | ccccgattacggggtgtcttggatcagacagccaccggggaagggtctggaatggattggagtga |
| | | tttggggctctgagactacttactacaactcatccctcaagtcacgcgtcaccatctcaaaggacaac |
| | | tctaagaatcaggtgtcactgaaactgtcatctgtgaccgcagccgacaccgccgtgtactattgcg |
| | | ctaagcattactattatggcgggagctacgcaatggattactggggacagggtactctggtcaccgt |
| | | gtccagcaccactaccccagcaccgaggccacccaccccggctcctaccatcgcctcccagcctc |
| | | tgtccctgcgtccggaggcatgtagacccgcagctggtggggccgtgcatacccggggtcttgact |
| | | tcgcctgcgatatctacatttgggcccctctggctggtacttgcggggtcctgctgctttcactcgtgat |
| | | cactctttactgtaagcgcggtcggaagaagctgctgtacatctttaagcaacccttcatgaggcctg |
| | | tgcagactactcaagaggaggacggctgttcatgccggttcccagaggaggaggaaggcggctg |
| | | cgaactgcgcgtgaaattcagccgcagcgcagatgctccagcctacaagcaggggcagaaccag |
| | | ctctacaacgaactcaatcttggtcggagagaggagtacgacgtgctggacaagcggagaggac |
| | | gggacccagaaatgggcgggaagccgcgcagaaagaatccccaagagggcctgtacaacgag |
| | | ctccaaaaggataagatggcagaagcctatagcgagattggtatgaaaggggaacgcagaagag |
| | | gcaaaggccacgacggactgtaccagggactcagcaccgccaccaaggacacctatgacgctct |
| | | tcacatgcaggccctgccgcctcgg |
| 105975 | 40 | MALPVTALLLPLALLLHAARPEIVMTQSPATLSLSPGERATLSC |
| CAR 10 | | RASQDISKYLNWYQQKPGQAPRLLIYHTSRLHSGIPARFSGSG |
| Full-aa | | SGTDYTLTISSLQPEDFAVYFCQQGNTLPYTFGQGTKLEIKGG |
| | | GGSGGGGSGGGGSGGGGSQVQLQESGPGLVKPSETLSLTCTVS |
| | | GVSLPDYGVSWIRQPPGKGLEWIGVIWGSETTYYNSSLKSRV |
| | | TISKDNSKNQVSLKLSSVTAADTAVYYCAKHYYYGGSYAMD |
| | | YWGQGTLVTVSSTTTPAPRPPTPAPTIASQPLSLRPEACRPAAG |
| | | GAVHTRGLDFACDIYIWAPLAGTCGVLLLSLVITLYCKRGRKK |
| | | LLYIFKQPFMRPVQTTQEEDGCSCRFPEEEEGGCELRVKFSRSA |
| | | DAPAYKQGQNQLYNELNLGRREEYDVLDKRRGRDPEMGGKP |
| | | RRKNPQEGLYNELQKDKMAEAYSEIGMKGERRRGKGHDGLY |
| | | QGLSTATKDTYDALHMQALPPR |

CAR11

| | | |
|---|---|---|
| CAR11 | 11 | eivmtqspatlslspgeratlscrasqdiskylnwyqqkpgqaprlliyhtsrlhsgiparfsgsgs |
| scFv | | gtdytltisslqpedfavyfcqqgntlpytfgqgtkleikggggsggggsggggsqvqlqesgpg |
| domain | | lvkpsetlsltctvsgvslpdygvswirqppgkglewigviwgsettyynsslksrvtiskdnskn |
| | | qvslklssvtaadtavyycakhyyyggsyamdywgqgtlvtvss |
| 103101 | 71 | Atggccctccctgtcaccgccctgctgcttccgctggctcttctgctccacgccgctcggcccgaa |
| CAR11- | | attgtgatgacccagtcacccgccactcttagcctttcacccggtgagcgcgcaaccctgtcttgca |
| Soluble | | gagcctcccaagacatctcaaaataccttaattggtatcaacagaagcccggacaggctcctcgcct |
| scFv-nt | | tctgatctaccacaccagccggctccattctggaatccctgccaggttcagcggtagcggatctggg |
| | | accgactacaccctcactatcagctcactgcagccagaggacttcgctgtctatttctgtcagcaagg |
| | | gaacaccctgccctacacctttggacagggcaccaagctcgagattaaaggtggaggtggcagcg |
| | | gaggaggtgggtccggcggtggaggaagccaggtccaactccaagaaagcggaccgggtcttg |
| | | tgaagccatcagaaactctttcactgacttgtactgtgagcggagtgtctctccccgattacggggtgt |
| | | cttggatcagacagccaccggggaagggtctggaatggattggagtgatttggggctctgagacta |
| | | cttactacaattcatccctcaagtcacgcgtcaccatctcaaaggacaactctaagaatcaggtgtca |
| | | ctgaaactgtcatctgtgaccgcagccgacaccgccgtgtactattgcgctaagcattactattatgg |
| | | cgggagctacgcaatggattactggggacagggtactctggtcaccgtgtccagccaccaccatc |
| | | atcaccatcaccat |
| 103101 | 83 | MALPVTALLLPLALLLHAARPeivmtqspatlslspgeratlscrasqdiskyl |
| CAR11- | | nwyqqkpgqaprlliyhtsrlhsgiparfsgsgsgtdytltisslqpedfavyfcqqgntlpytfgq |
| Soluble | | gtkleikggggsggggsggggsqvqlqesgpglvkpsetlsltctvsgvslpdygvswirqppg |
| scFv-aa | | kglewigviwgsettyynsslksrvtiskdnsknqvslklssvtaadtavyycakhyyyggsya |
| | | mdywgqgtlvtvsshhhhhhhh |
| 105976 | 95 | atggctctgcccgtgaccgcactcctcctgccactggctctgctgcttcacgccgctcgcccacaag |
| CAR 11 | | tccagcttcaagaatcagggcctggtctggtgaagccatctgagactctgtccctcacttgcaccgtg |
| Full-nt | | agcggagtgtccctcccagactacggagtgagctggattagacagcctcccggaaagggactgg |
| | | agtggatcggagtgatttggggtagcgaaaccacttactataactcttccctgaagtcacgggtcac |
| | | catttcaaaggataactcaaagaatcaagtgagcctcaagctctcatcagtcaccgccgctgacacc |
| | | gccgtgtattactgtgccaagcattactactatggagggtcctacgccatggactactggggccagg |
| | | gaactctggtcactgtgtcatctggtggaggaggtagcggaggaggcgggagcggtggaggtgg |
| | | ctccggaggtggcggaagcgaaatcgtgatgacccagagccctgcaaccctgtccctttctcccg |
| | | gggaacgggctaccctttcttgtcgggcatcacaagatatctcaaaatacctcaattggtatcaacag |
| | | aagccgggacaggcccctaggcttcttatctaccacacctctcgcctgcatagcgggattcccgca |
| | | cgctttagcgggtctggaagcgggaccgactacactctgaccatctcatctctccagcccgaggac |
| | | ttcgccgtctacttctgccagcagggtaacaccctgccgtacaccttcggccagggcaccaagcttg |
| | | agatcaaaaccactactcccgctccaaggccacccacccctgccccgaccatcgcctctcagccg |
| | | ctttccctgcgtccggaggcatgtagacccgcagctggtggggccgtgcatacccggggtcttgac |
| | | ttcgcctgcgatatctacatttgggcccctctggctggtacttgcggggtcctgctgctttcactcgtga |
| | | tcactctttactgtaagcgcggtcggaagaagctgctgtacatctttaagcaacccttcatgaggcct |
| | | gtgcagactactcaagaggaggacggctgttcatgccggttcccagaggaggaggaaggcggct |
| | | gcgaactgcgcgtgaaattcagccgcagcgcagatgctccagcctacaagcaggggcagaacca |
| | | gctctacaacgaactcaatcttggtcggagagaggagtacgacgtgctggacaagcggagagga |
| | | cgggacccagaaatgggcgggaagccgcgcagaaagaatccccaagagggcctgtacaacga |
| | | gctccaaaaggataagatggcagaagcctatagcgagattggtatgaaaggggaacgcagaaga |
| | | ggcaaaggccacgacggactgtaccagggactcagcaccgccaccaaggacacctatgacgct |
| | | cttcacatgcaggccctgccgcctcgg |
| 105976 | 41 | MALPVTALLLPLALLLHAARPQVQLQESGPGLVKPSETLSLTC |
| CAR 11 | | TVSGVSLPDYGVSWIRQPPGKGLEWIGVIWGSETTYYNSSLK |
| Full-aa | | SRVTISKDNSKNQVSLKLSSVTAADTAVYYCAKHYYYGGSYA |
| | | MDYWGQGTLVTVSSGGGGSGGGGSGGGGSGGGGSEIVMTQS |
| | | PATLSLSPGERATLSCRASQDISKYLNWYQQKPGQAPRLLIYH |
| | | TSRLHSGIPARFSGSGSGTDYTLTISSLQPEDFAVYFCQQGNTL |
| | | PYTFGQGTKLEIKTTTPAPRPPTPAPTIASQPLSLRPEACRPAAG |
| | | GAVHTRGLDFACDIYIWAPLAGTCGVLLLSLVITLYCKRGRKK |
| | | LLYIFKQPFMRPVQTTQEEDGCSCRFPEEEEGGCELRVKFSRSA |
| | | DAPAYKQGQNQLYNELNLGRREEYDVLDKRRGRDPEMGGKP |
| | | RRKNPQEGLYNELQKDKMAEAYSEIGMKGERRRGKGHDGLY |
| | | QGLSTATKDTYDALHMQALPPR |

CAR12

| | | |
|---|---|---|
| CAR12 | 12 | qvqlqesgpglvkpsetlsltctvsgvslpdygvswirqppgkglewigviwgsettyynsslks |
| scFv | | rvtiskdnsknqvslklssvtaadtavyycakhyyyggsyamdywgqgtlvtvssggggsgg |
| domain | | ggsggggseivmtqspatlslspgeratlscrasqdiskylnwyqqkpgqaprlliyhtsrlhsgi |
| | | parfsgsgsgtdytltisslqpedfavyfcqqgntlpytfgqgtkleik |
| 103104 | 72 | atggctctgcccgtgaccgcactcctcctgccactggctctgctgcttcacgccgctcgcccacaag |
| CAR12- | | tccagcttcaagaatcagggcctggtctggtgaagccatctgagactctgtccctcacttgcaccgtg |
| Soluble | | agcggagtgtccctcccagactacggagtgagctggattagacagcctcccggaaagggactgg |
| scFv-nt | | agtggatcggagtgatttggggtagcgaaaccacttactataactcttccctgaagtcacgggtcac |
| | | catttcaaaggataactcaaagaatcaagtgagcctcaagctctcatcagtcaccgccgctgacacc |
| | | gccgtgtattactgtgccaagcattactactatggagggtcctacgccatggactactggggccagg |
| | | gaactctggtcactgtgtcatctggtggaggaggtagcggaggaggcgggagcggtggaggtgg |
| | | ctccgaaatcgtgatgacccagagccctgcaaccctgtccctttctcccggggaacgggctaccctt |
| | | tcttgtcgggcatcacaagatatctcaaaatacctcaattggtatcaacagaagccgggacaggccc |
| | | ctaggcttcttatctaccacacctctcgcctgcatagcgggattcccgcacgctttagcgggtctgga |
| | | agcgggaccgactacactctgaccatctcatctctccagcccgaggacttcgccgtctacttctgcc |
| | | agcagggtaacaccctgccgtacaccttcggccagggcaccaagcttgagatcaaacatcaccac |
| | | catcatcaccatcac |
| 103104 | 84 | MALPVTALLLPLALLLHAARPqvqlqesgpglvkpsetlsltctvsgvslpdy |
| CAR12- | | gvswirqppgkglewigviwgsettyynsslksrvtiskdnsknqvslklssvtaadtavyyca |
| Soluble | | khyyyggsyamdywgqgtlvtvssggggsggggsggggseivmtqspatlslspgeratlscr |
| scFv-aa | | asqdiskylnwyqqkpgqaprlliyhtsrlhsgiparfsgsgsgtdytltisslqpedfavyfcqqg |
| | | ntlpytfgqgtkleikhhhhhhhh |
| 105977 | 96 | atggccctccctgtcaccgccctgctgcttccgctggctcttctgctccacgccgctcggcccgaaa |
| CAR 12- | | ttgtgatgacccagtcacccgccactcttagcctttcacccggtgagcgcgcaaccctgtcttgcag |
| Full-nt | | agcctcccaagacatctcaaaataccttaattggtatcaacagaagcccggacaggctcctcgcctt |
| | | ctgatctaccacaccagccggctccattctggaatccctgccaggttcagcggtagcggatctggg |
| | | accgactacaccctcactatcagctcactgcagccagaggacttcgctgtctatttctgtcagcaagg |
| | | gaacaccctgccctacacctttggacagggcaccaagctcgagattaaaggtggaggtggcagcg |
| | | gaggaggtgggtccggcggtggaggaagccaggtccaactccaagaaagcggaccgggtcttg |
| | | tgaagccatcagaaactctttcactgacttgtactgtgagcggagtgtctctccccgattacggggtgt |
| | | cttggatcagacagccaccggggaagggtctggaatggattggagtgatttggggctctgagacta |
| | | cttactacaactcatccctcaagtcacgcgtcaccatctcaaaggacaactctaagaatcaggtgtca |
| | | ctgaaactgtcatctgtgaccgcagccgacaccgccgtgtactattgcgctaagcattactattatgg |
| | | cgggagctacgcaatggattactggggacagggtactctggtcaccgtgtccagcaccactaccc |
| | | cagcaccgaggccacccaccccggctcctaccatcgcctcccagcctctgtccctgcgtccggag |
| | | gcatgtagacccgcagctggtggggccgtgcatacccggggtcttgacttcgcctgcgatatctac |
| | | atttgggcccctctggctggtacttgcggggtcctgctgctttcactcgtgatcactctttactgtaagc |
| | | gcggtcggaagaagctgctgtacatctttaagcaacccttcatgaggcctgtgcagactactcaaga |
| | | ggaggacggctgttcatgccggttcccagaggaggaggaaggcggctgcgaactgcgcgtgaa |
| | | attcagccgcagcgcagatgctccagcctacaagcaggggcagaaccagctctacaacgaactc |
| | | aatcttggtcggagagaggagtacgacgtgctggacaagcggagaggacgggacccagaaatg |
| | | ggcgggaagccgcgcagaaagaatccccaagagggcctgtacaacgagctccaaaaggataag |
| | | atggcagaagcctatagcgagattggtatgaaaggggaacgcagaagaggcaaaggccacgac |
| | | ggactgtaccagggactcagcaccgccaccaaggacacctatgacgctcttcacatgcaggccct |
| | | gccgcctcgg |
| 105977 | 42 | MALPVTALLLPLALLLHAARPEIVMTQSPATLSLSPGERATLSC |
| CAR 12- | | RASQDISKYLNWYQQKPGQAPRLLIYHTSRLHSGIPARFSGSG |
| Full-aa | | SGTDYTLTISSLQPEDFAVYFCQQGNTLPYTFGQGTKLEIKGG |
| | | GGSGGGGSGGGGSQVQLQESGPGLVKPSETLSLTCTVSGVSLP |
| | | DYGVSWIRQPPGKGLEWIGVIWGSETTYYNSSLKSRVTISKD |
| | | NSKNQVSLKLSSVTAADTAVYYCAKHYYYGGSYAMDYWGQ |
| | | GTLVTVSSTTTPAPRPPTPAPTIASQPLSLRPEACRPAAGGAVH |
| | | TRGLDFACDIYIWAPLAGTCGVLLLSLVITLYCKRGRKKLLYIF |
| | | KQPFMRPVQTTQEEDGCSCRFPEEEEGGCELRVKFSRSADAPA |
| | | YKQGQNQLYNELNLGRREEYDVLDKRRGRDPEMGGKPRRKN |
| | | PQEGLYNELQKDKMAEAYSEIGMKGERRRGKGHDGLYQGLS |
| | | TATKDTYDALHMQALPPR |

TABLE 3
Murine CD19 CAR Constructs
CTL019

| | | |
|---|---|---|
| CTL019- | 97 | Atggccctgcccgtcaccgctctgctgctgccccttgctctgcttcttcatgcagcaaggccggaca |
| Soluble | | tccagatgacccaaaccacctcatccctctctgcctctcttggagacagggtgaccatttcttgtcgc |
| scFv- | | gccagccaggacatcagcaagtatctgaactggtatcagcagaagccggacggaaccgtgaagc |
| Histag-nt | | tcctgatctaccatacctctcgcctgcatagcggcgtgccctcacgcttctctggaagcggatcagg |
| | | aaccgattattctctcactatttcaaatcttgagcaggaagatattgccacctatttctgccagcagggt |
| | | aataccctgccctacaccttcggaggagggaccaagctcgaaatcaccggtggaggaggcagcg |
| | | gcggtggagggtctggtggaggtggttctgaggtgaagctgcaagaatcaggccctggacttgtg |
| | | gccccttcacagtccctgagcgtgacttgcaccgtgtccggagtctccctgcccgactacggagtgt |
| | | catggatcagacaacctccacggaaaggactggaatggctcggtgtcatctggggtagcgaaact |
| | | acttactacaattcagccctcaaaagcaggctgactattatcaaggacaacagcaagtcccaagtctt |
| | | tcttaagatgaactcactccagactgacgacaccgcaatctactattgtgctaagcactactactacg |
| | | gaggatcctacgctatggattactggggacaaggtacttccgtcactgtctcttcacaccatcatcac |
| | | catcaccatcac |
| CTL019- | 98 | MALPVTALLLPLALLLHAARPdiqmtqttsslsaslgdrvtiscrasqdiskyl |
| Soluble | | nwyqqkpdgtvklliyhtsrlhsgvpsrfsgsgsgtdysltisnleqediatyfcqqgntlpytfgg |
| scFv- | | gtkleitggggsggggsggggsevklqesgpglvapsqslsvtctvsgvslpdygvswirqppr |
| Histag-aa | | kglewlgviwgsettyynsalksrltiikdnsksqvflkmnslqtddtaiyycakhyyyggsya |
| | | mdywgqgtsvtvsshhhhhhhh |
| CTL019 | 99 | atggccttaccagtgaccgccttgctcctgccgctggccttgctgctccacgccgccaggccggac |
| Full-nt | | atccagatgacacagactacatcctccctgtctgcctctctgggagacagagtcaccatcagttgca |
| | | gggcaagtcaggacattagtaaatatttaaattggtatcagcagaaaccagatggaactgttaaactc |
| | | ctgatctaccatacatcaagattacactcaggagtcccatcaaggttcagtggcagtgggtctggaa |
| | | cagattattctctcaccattagcaacctggagcaagaagatattgccacttacttttgccaacagggta |
| | | atacgcttccgtacacgttcggaggggggaccaagctggagatcacaggtggcggtggctcggg |
| | | cggtggtgggtcgggtggcggcggatctgaggtgaaactgcaggagtcaggacctggcctggtg |
| | | gcgccctcacagagcctgtccgtcacatgcactgtctcaggggtctcattacccgactatggtgtaa |
| | | gctggattcgccagcctccacgaaagggtctggagtggctgggagtaatatggggtagtgaaacc |
| | | acatactataattcagctctcaaatccagactgaccatcatcaaggacaactccaagagccaagtttt |
| | | cttaaaaatgaacagtctgcaaactgatgacacagccatttactactgtgccaaacattattactacgg |
| | | tggtagctatgctatggactactggggccaaggaacctcagtcaccgtctcctcaaccacgacgcc |
| | | agcgccgcgaccaccaacaccggcgcccaccatcgcgtcgcagcccctgtccctgcgcccaga |
| | | ggcgtgccggccagcggcggggggcgcagtgcacacgagggggctggacttcgcctgtgatat |
| | | ctacatctgggcgcccttggccgggacttgtggggtccttctcctgtcactggttatcaccctttactg |
| | | caaacggggcagaaagaaactcctgtatatattcaaacaaccatttatgagaccagtacaaactact |
| | | caagaggaagatggctgtagctgccgatttccagaagaagaagaaggaggatgtgaactgagagt |
| | | gaagttcagcaggagcgcagacgcccccgcgtacaagcagggccagaaccagctctataacga |
| | | gctcaatctaggacgaagagaggagtacgatgttttggacaagagacgtggccgggaccctgaga |
| | | tggggggaaagccgagaaggaagaaccctcaggaaggcctgtacaatgaactgcagaaagata |
| | | agatggcggaggcctacagtgagattgggatgaaaggcgagcgccggaggggcaaggggcac |
| | | gatggcctttaccagggtctcagtacagccaccaaggacacctacgacgcccttcacatgcaggcc |
| | | ctgccccctcgc |
| CTL019 | 58 | MALPVTALLLPLALLLHAARPdiqmtqttsslsaslgdrvtiscrasqdiskylnw |
| Full-aa | | yqqkpdgtvklliyhtsrlhsgvpsrfsgsgsgtdysltisnleqediatyfcqqgntlpytfgggtk |
| | | leitggggsggggsggggsevklqesgpglvapsqslsvtctvsgvslpdygvswirqpprkgl |
| | | ewlgviwgsettyynsalksrltiikdnsksqvflkmnslqtddtaiyycakhyyyggsyamd |
| | | ywgqgtsvtvsstttpaprpptpaptiasqplslrpeacrpaaggavhtrgldfacdiyiwaplagt |
| | | cgvlllslvitlyckrgrkkllyifkqpfmrpvqttqeedgcscrfpeeeeggcelrvkfsrsadap |
| | | aykqgqnqlynelnlgrreeydvldkrrgrdpemggkprrknpqeglynelqkdkmaeayse |
| | | igmkgerrrgkghdglyqglstatkdtydalhmqalppr |
| CTL019 | 59 | Diqmtqttsslsaslgdrvtiscrasqdiskylnwyqqkpdgtvklliyhtsrlhsgvpsrfsgsgs |
| scFv | | gtdysltisnleqediatyfcqqgntlpytfgggtkleitggggsggggsggggsevklqesgpgl |
| domain | | vapsqslsvtctvsgvslpdygvswirqpprkglewlgviwgsettyynsalksrltiikdnsksq |
| | | vflkmnslqtddtaiyycakhyyyggsyamdywgqgtsvtvss |
| mCAR1 | 109 | QVQLLESGAELVRPGSSVKISCKASGYAFSSYWMNWVKQRPG |
| scFv | | QGLEWIGQIYPGDGDTNYNGKFKGQATLTADKSSSTAYMQLS |
| | | GLTSEDSAVYSCARKTISSVVDFYFDYWGQGTTVTGGGSGGG |
| | | SGGGSGGGSELVLTQSPKFMSTSVGDRVSVTCKASQNVGTNV |
| | | AWYQQKPGQSPKPLIYSATYRNSGVPDRFTGSGSGTDFTLTIT |
| | | NVQSKDLADYFCQYNRYPYTSFFFTKLEIKRRS |
| mCAR1 | 110 | QVQLLESGAELVRPGSSVKISCKASGYAFSSYWMNWVKQRPG |
| Full-aa | | QGLEWIGQIYPGDGDTNYNGKFKGQATLTADKSSSTAYMQLS |
| | | GLTSEDSAVYSCARKTISSVVDFYFDYWGQGTTVTGGGSGGG |
| | | SGGGSGGGSELVLTQSPKFMSTSVGDRVSVTCKASQNVGTNV |
| | | AWYQQKPGQSPKPLIYSATYRNSGVPDRFTGSGSGTDFTLTIT |
| | | NVQSKDLADYFCQYNRYPYTSFFFTKLEIKRRSKIEVMYPPPYL |
| | | DNEKSNGTIIHVKGKHLCPSPLFPGPSKPFWVLVVVGGVLACY |
| | | SLLVTVAFIIFWVRSKRSRLLHSDYMNMTPRRPGPTRKHYQPY |
| | | APPRDFAAYRSRVKFSRSADAPAYQQGQNQLYNELNLGRREE |
| | | YDVLDKRRGRDPEMGGKPRRKNPQEGLYNELQKDKMAEAYS |
| | | EIGMKGERRRGKGHDGLYQGLSTATKDTYDALHMQALPPR |
| mCAR2 | 111 | DIQMTQTTSSLSASLGDRVTISCRASQDISKYLNWYQQKPDGT |
| scFv | | VKLLIYHTSRLHSGVPSRFSGSGSGTDYSLTISNLEQEDIATYFC |
| | | QQGNTLPYTFGGGTKLEITGSTSGSGKPGSGEGSTKGEVKLQE |
| | | SGPGLVAPSQSLSVTCTVSGVSLPDYGVSWIRQPPRKGLEWLG |
| | | VIWGSETTYYNSALKSRLTIIKDNSKSQVFLKMNSLQTDDTAIY |
| | | YCAKHYYYGGSYAMDYWGQGTSVTVSSE |
| mCAR2 | 112 | DIQMTQTTSSLSASLGDRVTISCRASQDISKYLNWYQQKPDGT |
| CAR-aa | | VKLLIYHTSRLHSGVPSRFSGSGSGTDYSLTISNLEQEDIATYFC |
| | | QQGNTLPYTFGGGTKLEITGSTSGSGKPGSGEGSTKGEVKLQE |
| | | SGPGLVAPSQSLSVTCTVSGVSLPDYGVSWIRQPPRKGLEWLG |
| | | VIWGSETTYYNSALKSRLTIIKDNSKSQVFLKMNSLQTDDTAIY |
| | | YCAKHYYYGGSYAMDYWGQGTSVTVSSESKYGPPCPPCPMF |
| | | WVLVVVGGVLACYSLLVTVAFIIFWVKRGRKKLLYIFKQPFM |
| | | RPVQTTQEEDGCSCRFEEEEGGCELRVKFSRSADAPAYQQGQ |
| | | NQLYNELNLGRREEYDVLDKRRGRDPEMGGKPRRKNPQEGL |
| | | YNELQKDKMAEAYSEIGMKGERRRGKGHDGLYQGLSTATKD |
| | | TYDALHMQALPPRL |
| mCAR2 | 113 | DIQMTQTT SSLSASLGDR VTISCRASQD ISKYLNWYQQ |
| Full-aa | | KPDGTVKLLI YHTSRLHSGV PSRFSGSGSG TDYSLTISNL |
| | | EQEDIATYFC QQGNTLPYTF GGGTKLEITG STSGSGKPGS |
| | | GEGSTKGEVK LQESGPGLVA PSQSLSVTCT VSGVSLPDYG |
| | | VSWIRQPPRK GLEWLGVIWG SETTYYNSAL KSRLTIIKDN |
| | | SKSQVFLKMN SLQTDDTAIY YCAKHYYYGG |
| | | SYAMDYWGQG TSVTVSSESK YGPPCPPCPM FWVLVVVGGV |
| | | LACYSLLVTV |
| | | AFIIFWVKRG RKKLLYIFKQ PFMRPVQTTQ EEDGCSCRFE |
| | | EEEGGCELRV KFSRSADAPA YQQGQNQLYN ELNLGRREEY |
| | | DVLDKRRGRD PEMGGKPRRK NPQEGLYNEL QKDKMAEAYS |
| | | EIGMKGERRR GKGHDGLYQG LSTATKDTYD ALHMQALPPR |
| | | LEGGGEGRGS LLTCGDVEEN PGPRMLLLVT SLLLCELPHP |
| | | AFLLIPRKVC NGIGIGEFKD SLSINATNIK HFKNCTSISG |
| | | DLHILPVAFR GDSFTHTPPL DPQELDILKT VKEITGFLLI |
| | | QAWPENRTDL HAFENLEIIR |
| | | GRTKQHGQFS LAVVSLNITS LGLRSLKEIS DGDVIISGNK |
| | | NLCYANTINW KKLFGTSGQK TKIISNRGEN SCKATGQVCH |
| | | ALCSPEGCWG PEPRDCVSCR NVSRGRECVD KCNLLEGEPR |
| | | EFVENSECIQ CHPECLPQAM NITCTGRGPD NCIQCAHYID |
| | | GPHCVKTCPA GVMGENNTLV WKYADAGHVC |
| | | HLCHPNCTYG CTGPGLEGCP TNGPKIPSIA TGMVGALLLL |
| | | LVVALGIGLF M |
| mCAR3 | 114 | DIQMTQTTSSLSASLGDRVTISCRASQDISKYLNWYQQKPDGT |
| scFv | | VKLLIYHTSRLHSGVPSRFSGSGSGTDYSLTISNLEQEDIATYFC |
| | | QQGNTLPYTFGGGTKLEITGSTSGSGKPGSGEGSTKGEVKLQE |
| | | SGPGLVAPSQSLSVTCTVSGVSLPDYGVSWIRQPPRKGLEWLG |
| | | VIWGSETTYYNSALKSRLTIIKDNSKSQVFLKMNSLQTDDTAIY |
| | | YCAKHYYYGGSYAMDYWGQGTSVTVSS |
| mCAR3 | 115 | DIQMTQTTSSLSASLGDRVTISCRASQDISKYLNWYQQKPDGT |
| Full-aa | | VKLLIYHTSRLHSGVPSRFSGSGSGTDYSLTISNLEQEDIATYFC |
| | | QQGNTLPYTFGGGTKLEITGSTSGSGKPGSGEGSTKGEVKLQE |
| | | SGPGLVAPSQSLSVTCTVSGVSLPDYGVSWIRQPPRKGLEWLG |
| | | VIWGSETTYYNSALKSRLTIIKDNSKSQVFLKMNSLQTDDTAIY |
| | | YCAKHYYYGGSYAMDYWGQGTSVTVSSAAAIEVMYPPPYLD |
| | | NEKSNGTIIHVKGKHLCPSPLFPGPSKPFWVLVVVGGVLACYS |
| | | LLVTVAFIIFWVRSKRSRLLHSDYMNMTPRRPGPTRKHYQPYA |
| | | PPRDFAAYRSRVKFSRSADAPAYQQGQNQLYNELNLGRREEY |
| | | DVLDKRRGRDPEMGGKPRRKNPQEGLYNELQKDKMAEAYSE |
| | | IGMKGERRRGKGHDGLYQGLSTATKDTYDALHMQALPPR |
| SSJ25-C1 | | QVQLLESGAELVRPGSSVKISCKASGYAFSSYWMNWVKQRPG |
| VH | | QGLEWIGQIYPGDGDTNYNGKFKGQATLTADKSSSTAYMQLS |
| sequence | | GLTSEDSAVYSCARKTISSVVDFYFDYWGQGTTVT |
| SSJ25-C1 | | ELVLTQSPKFMSTSVGDRVSVTCKASQNVGTNVAWYQQKPG |
| VL | | QSPKPLIYSATYRNSGVPDRFTGSGSGTDFTLTITNVQSKDLAD |
| sequence | | YFYFCQYNRYPYTSGGGTKLEIKRRS |

TABLE 4
Heavy Chain Variable Domain CDRs

| Candidate | FW | HCDR1 | ID | HCDR2 | ID | HCDR3 | ID |
|---|---|---|---|---|---|---|---|
| murine_CART19 | | GVSLPDYGVS | 19 | VIWGSETTYYNSALKS | 20 | HYYYGGSYAMDY | 24 |
| humanized_CART19 a | VH4 | GVSLPDYGVS | 19 | VIWGSETTYYSSSLKS | 21 | HYYYGGSYAMDY | 24 |
| humanized_CART19 b | VH4 | GVSLPDYGVS | 19 | VIWGSETTYYQSSLKS | 22 | HYYYGGSYAMDY | 24 |
| humanized_CART19 c | VH4 | GVSLPDYGVS | 19 | VIWGSETTYYNSSLKS | 23 | HYYYGGSYAMDY | 24 |

TABLE 5
Light Chain Variable Domain CDRs

| Candidate | FW | LCDR1 | ID | LCDR2 | ID | LCDR3 | ID |
|---|---|---|---|---|---|---|---|
| murine_CART19 | | RASQDISKYLN | 25 | HTSRLHS | 26 | QQGNTLPYT | 27 |
| humanized_CART19 a | VK3 | RASQDISKYLN | 25 | HTSRLHS | 26 | QQGNTLPYT | 27 |
| humanized_CART19 b | VK3 | RASQDISKYLN | 25 | HTSRLHS | 26 | QQGNTLPYT | 27 |
| humanized_CART19 c | VK3 | RASQDISKYLN | 25 | HTSRLHS | 26 | QQGNTLPYT | 27 |

Provided herein are CD19 inhibitors and combination therapies. In some embodiments, the CD19 inhibitor (e.g., a cell therapy, e.g., a CD19-expressing CAR, or an antibody) is administered in combination with a B cell inhibitor, e.g., one or more inhibitors of CD10, CD19, CD20, CD22, CD34, CD123, FLT-3, or ROR1. A CD19 inhibitor includes but is not limited to a CD19 CAR-expressing cell, e.g., a CD19 CART cell, or an anti-CD19 antibody (e.g., an anti-CD19 mono- or bispecific antibody) or a fragment or conjugate thereof. In an embodiment, the CD19 inhibitor is administered in combination with a B-cell inhibitor, e.g., a CAR-expressing cell described herein.

In some other embodiments, the CD19 inhibitor is administered in combination with a B-cell inhibitor, and their use in medicaments or methods for treating, among other diseases, cancer or any malignancy or autoimmune diseases involving cells or tissues which express CD19.

Numerous CD19 CAR-expressing cells are described in this disclosure. For instance, in some embodiments, a CD19 inhibitor includes an anti-CD19 CAR-expressing cell, e.g., CART, e.g., a cell expressing an anti-CD19 CAR construct described in Table 2, e.g., CTL119, or encoded by a CD19 binding CAR comprising a scFv, CDRs, or VH and VL chains described in Tables 2, 4, or 5. For example, an anti-CD19 CAR-expressing cell, e.g., CART, is a generated by engineering a CD19-CAR (that comprises a CD19 binding domain) into a cell (e.g., a T cell or NK cell), e.g., for administration in combination with a CAR-expressing cell described herein. Also provided herein are methods of use of the CAR-expressing cells described herein for adoptive therapy.

### BCMA Binding Domains and CARs

In embodiments the BCMA CAR comprises an anti-BCMA binding domain (e.g., human or humanized anti-BCMA binding domain), a transmembrane domain, and an intracellular signaling domain, and wherein said anti-BCMA binding domain comprises a heavy chain complementary determining region 1 (HC CDR1), a heavy chain complementary determining region 2 (HC CDR2), and a heavy chain complementary determining region 3 (HC CDR3) of any anti-BMCA heavy chain binding domain amino acid sequences listed in Table 4D or 4E.

In one embodiment, the anti-BCMA binding domain comprises a light chain variable region described herein (e.g., in Table 4D or 4E) and/or a heavy chain variable region described herein (e.g., in Table 4D or 4E).

In one embodiment, the encoded anti-BCMA binding domain is a scFv comprising a light chain and a heavy chain of an amino acid sequence of Table 4D or 4E.

In an embodiment, the human or humanized anti-BCMA binding domain (e.g., an scFv) comprises: a light chain variable region comprising an amino acid sequence having at least one, two or three modifications (e.g., substitutions, e.g., conservative substitutions) but not more than 30, 20 or 10 modifications (e.g., substitutions, e.g., conservative substitutions) of an amino acid sequence of a light chain variable region provided in Table 4D or 4E, or a sequence with at least 95% (e.g., 95-99%) identity thereof; and/or a heavy chain variable region comprising an amino acid sequence having at least one, two or three modifications (e.g., substitutions, e.g., conservative substitutions) but not more than 30, 20 or 10 modifications (e.g., substitutions, e.g., conservative substitutions) of an amino acid sequence of a heavy chain variable region provided in Table 4D or 4E, or a sequence with at least 95% (e.g., 95-99%) identity thereof.

TABLE 4D
Amino Acid and Nucleic Acid Sequences of exemplary
anti-BCMA scFv domains and BCMA CAR molecules

| | | |
|---|---|---|
| | SEQ | |
| Name/ | ID | |
| Description | NO: | Sequence |

139109

| | | |
|---|---|---|
| 139109-aa | 1400 | EVQLVESGGGLVQPGGSLRLSCAVSGFALSNHGMSWVRRAPGK |
| ScFv | | GLEWVSGIVYSGSTYYAASVKGRFTISRDNSRNTLYLQMNSLRP |
| domain | | EDTAIYYCSAHGGESDVWGQGTTVTVSSASGGGGSGGRASGGG |
| | | GSDIQLTQSPSSLSASVGDRVTITCRASQSISSYLNWYQQKPGKA |
| | | PKLLIYAASSLQSGVPSRFSGSGSGTDFTLTISSLQPEDFATYYCQ |
| | | QSYSTPYTFGQGTKVEIK |
| 139109-nt | 1401 | GAAGTGCAATTGGTGGAATCAGGGGGAGGACTTGTGCAGCCT |
| ScFv | | GGAGGATCGCTGAGACTGTCATGTGCCGTGTCCGGCTTTGCCC |
| domain | | TGTCCAACCACGGGATGTCCTGGGTCCGCCGCGCGCCTGGAA |
| | | AGGGCCTCGAATGGGTGTCGGGTATTGTGTACAGCGGTAGCA |
| | | CCTACTATGCCGCATCCGTGAAGGGGAGATTCACCATCAGCC |
| | | GGGACAACTCCAGGAACACTCTGTACCTCCAAATGAATTCGC |
| | | TGAGGCCAGAGGACACTGCCATCTACTACTGCTCCGCGCATG |
| | | GCGGAGAGTCCGACGTCTGGGGACAGGGGACCACCGTGACC |
| | | GTGTCTAGCGCGTCCGGCGGAGGCGGCAGCGGGGGTCGGGCA |
| | | TCAGGGGGCGGCGGATCGGACATCCAGCTCACCCAGTCCCCG |
| | | AGCTCGCTGTCCGCCTCCGTGGGAGATCGGGTCACCATCACG |
| | | TGCCGCGCCAGCCAGTCGATTTCCTCCTACCTGAACTGGTACC |
| | | AACAGAAGCCCGGAAAAGCCCCGAAGCTTCTCATCTACGCCG |
| | | CCTCGAGCCTGCAGTCAGGAGTGCCCTCACGGTTCTCCGGCTC |
| | | CGGTTCCGGTACTGATTTCACCCTGACCATTTCCTCCCTGCAA |
| | | CCGGAGGACTTCGCTACTTACTACTGCCAGCAGTCGTACTCCA |
| | | CCCCCTACACTTTCGGACAAGGCACCAAGGTCGAAATCAAG |
| 139109-aa | 1402 | EVQLVESGGGLVQPGGSLRLSCAVSGFALSNHGMSWVRRAPGK |
| VH | | GLEWVSGIVYSGSTYYAASVKGRFTISRDNSRNTLYLQMNSLRP |
| | | EDTAIYYCSAHGGESDVWGQGTTVTVSS |
| 139109-aa | 1403 | DIQLTQSPSSLSASVGDRVTITCRASQSISSYLNWYQQKPGKAPK |
| VL | | LLIYAASSLQSGVPSRFSGSGSGTDFTLTISSLQPEDFATYYCQQS |
| | | YSTPYTFGQGTKVEIK |
| 139109-aa | 1404 | MALPVTALLLPLALLLHAARPEVQLVESGGGLVQPGGSLRLSCA |
| Full CAR | | VSGFALSNHGMSWVRRAPGKGLEWVSGIVYSGSTYYAASVKGR |
| | | FTISRDNSRNTLYLQMNSLRPEDTAIYYCSAHGGESDVWGQGTT |
| | | VTVSSASGGGGSGGRASGGGGSDIQLTQSPSSLSASVGDRVTITC |
| | | RASQSISSYLNWYQQKPGKAPKLLIYAASSLQSGVPSRFSGSGSG |
| | | TDFTLTISSLQPEDFATYYCQQSYSTPYTFGQGTKVEIKTTTPAPR |
| | | PPTPAPTIASQPLSLRPEACRPAAGGAVHTRGLDFACDIYIWAPL |
| | | AGTCGVLLLSLVITLYCKRGRKKLLYIFKQPFMRPVQTTQEEDG |
| | | CSCRFPEEEEGGCELRVKFSRSADAPAYKQGQNQLYNELNLGRR |
| | | EEYDVLDKRRGRDPEMGGKPRRKNPQEGLYNELQKDKMAEAY |
| | | SEIGMKGERRRGKGHDGLYQGLSTATKDTYDALHMQALPPR |
| 139109-nt | 1405 | ATGGCCCTCCCTGTCACCGCCCTGCTGCTTCCGCTGGCTCTTC |
| Full CAR | | TGCTCCACGCCGCTCGGCCCGAAGTGCAATTGGTGGAATCAG |
| | | GGGGAGGACTTGTGCAGCCTGGAGGATCGCTGAGACTGTCAT |
| | | GTGCCGTGTCCGGCTTTGCCCTGTCCAACCACGGGATGTCCTG |
| | | GGTCCGCCGCGCGCCTGGAAAGGGCCTCGAATGGGTGTCGGG |
| | | TATTGTGTACAGCGGTAGCACCTACTATGCCGCATCCGTGAA |
| | | GGGGAGATTCACCATCAGCCGGGACAACTCCAGGAACACTCT |
| | | GTACCTCCAAATGAATTCGCTGAGGCCAGAGGACACTGCCAT |
| | | CTACTACTGCTCCGCGCATGGCGGAGAGTCCGACGTCTGGGG |
| | | ACAGGGGACCACCGTGACCGTGTCTAGCGCGTCCGGCGGAGG |
| | | CGGCAGCGGGGGTCGGGCATCAGGGGGCGGCGGATCGGACA |
| | | TCCAGCTCACCCAGTCCCCGAGCTCGCTGTCCGCCTCCGTGGG |
| | | AGATCGGGTCACCATCACGTGCCGCGCCAGCCAGTCGATTTC |
| | | CTCCTACCTGAACTGGTACCAACAGAAGCCCGGAAAAGCCCC |
| | | GAAGCTTCTCATCTACGCCGCCTCGAGCCTGCAGTCAGGAGT |
| | | GCCCTCACGGTTCTCCGGCTCCGGTTCCGGTACTGATTTCACC |
| | | CTGACCATTTCCTCCCTGCAACCGGAGGACTTCGCTACTTACT |
| | | ACTGCCAGCAGTCGTACTCCACCCCCTACACTTTCGGACAAG |
| | | GCACCAAGGTCGAAATCAAGACCACTACCCCAGCACCGAGGC |
| | | CACCCACCCCGGCTCCTACCATCGCCTCCCAGCCTCTGTCCCT |
| | | GCGTCCGGAGGCATGTAGACCCGCAGCTGGTGGGGCCGTGCA |
| | | TACCCGGGGTCTTGACTTCGCCTGCGATATCTACATTTGGGCC |
| | | CCTCTGGCTGGTACTTGCGGGGTCCTGCTGCTTTCACTCGTGA |
| | | TCACTCTTTACTGTAAGCGCGGTCGGAAGAAGCTGCTGTACAT |
| | | CTTTAAGCAACCCTTCATGAGGCCTGTGCAGACTACTCAAGA |
| | | GGAGGACGGCTGTTCATGCCGGTTCCCAGAGGAGGAGGAAG |
| | | GCGGCTGCGAACTGCGCGTGAAATTCAGCCGCAGCGCAGATG |
| | | CTCCAGCCTACAAGCAGGGGCAGAACCAGCTCTACAACGAAC |
| | | TCAATCTTGGTCGGAGAGAGGAGTACGACGTGCTGGACAAGC |
| | | GGAGAGGACGGGACCCAGAAATGGGCGGGAAGCCGCGCAGA |
| | | AAGAATCCCCAAGAGGGCCTGTACAACGAGCTCCAAAAGGAT |
| | | AAGATGGCAGAAGCCTATAGCGAGATTGGTATGAAAGGGGA |
| | | ACGCAGAAGAGGCAAAGGCCACGACGGACTGTACCAGGGAC |
| | | TCAGCACCGCCACCAAGGACACCTATGACGCTCTTCACATGC |
| | | AGGCCCTGCCGCCTCGG |

139103

| | | |
|---|---|---|
| 139103-aa | 1406 | QVQLVESGGGLVQPGRSLRLSCAASGFTFSNYAMSWVRQAPGK |
| ScFv | | GLGWVSGISRSGENTYYADSVKGRFTISRDNSKNTLYLQMNSLR |
| domain | | DEDTAVYYCARSPAHYYGGMDVWGQGTTVTVSSASGGGGSGG |
| | | RASGGGGSDIVLTQSPGTLSLSPGERATLSCRASQSISSSFLAWYQ |
| | | QKPGQAPRLLIYGASRRATGIPDRFSGSGSGTDFTLTISRLEPEDS |
| | | AVYYCQQYHSSPSWTFGQGTKLEIK |
| 139103-nt | 1407 | CAAGTGCAACTCGTGGAATCTGGTGGAGGACTCGTGCAACCC |
| ScFv | | GGAAGATCGCTTAGACTGTCGTGTGCCGCCAGCGGGTTCACT |
| domain | | TTCTCGAACTACGCGATGTCCTGGGTCCGCCAGGCACCCGGA |
| | | AAGGGACTCGGTTGGGTGTCCGGCATTTCCCGGTCCGGCGAA |
| | | AATACCTACTACGCCGACTCCGTGAAGGGCCGCTTCACCATCT |
| | | CAAGGGACAACAGCAAAAACACCCTGTACTTGCAAATGAACT |
| | | CCCTGCGGGATGAAGATACAGCCGTGTACTATTGCGCCCGGT |
| | | CGCCTGCCCATTACTACGGCGGAATGGACGTCTGGGGACAGG |
| | | GAACCACTGTGACTGTCAGCAGCGCGTCGGGTGGCGGCGGCT |
| | | CAGGGGGTCGGGCCTCCGGGGGGGGAGGGTCCGACATCGTGC |
| | | TGACCCAGTCCCCGGGAACCCTGAGCCTGAGCCCGGGAGAGC |
| | | GCGCGACCCTGTCATGCCGGGCATCCCAGAGCATTAGCTCCT |
| | | CCTTTCTCGCCTGGTATCAGCAGAAGCCCGGACAGGCCCCGA |
| | | GGCTGCTGATCTACGGCGCTAGCAGAAGGGCTACCGGAATCC |
| | | CAGACCGGTTCTCCGGCTCCGGTTCCGGGACCGATTTCACCCT |
| | | TACTATCTCGCGCCTGGAACCTGAGGACTCCGCCGTCTACTAC |
| | | TGCCAGCAGTACCACTCATCCCCGTCGTGGACGTTCGGACAG |
| | | GGCACCAAGCTGGAGATTAAG |
| 139103-aa | 1408 | QVQLVESGGGLVQPGRSLRLSCAASGFTFSNYAMSWVRQAPGK |
| VH | | GLGWVSGISRSGENTYYADSVKGRFTISRDNSKNTLYLQMNSLR |
| | | DEDTAVYYCARSPAHYYGGMDVWGQGTTVTVSS |
| 139103-aa | 1409 | DIVLTQSPGTLSLSPGERATLSCRASQSISSSFLAWYQQKPGQAPR |
| VL | | LLIYGASRRATGIPDRFSGSGSGTDFTLTISRLEPEDSAVYYCQQY |
| | | HSSPSWTFGQGTKLEIK |
| 139103-aa | 1410 | MALPVTALLLPLALLLHAARPQVQLVESGGGLVQPGRSLRLSCA |
| Full CAR | | ASGFTFSNYAMSWVRQAPGKGLGWVSGISRSGENTYYADSVKG |
| | | RFTISRDNSKNTLYLQMNSLRDEDTAVYYCARSPAHYYGGMDV |
| | | WGQGTTVTVSSASGGGGSGGRASGGGGSDIVLTQSPGTLSLSPG |
| | | ERATLSCRASQSISSSFLAWYQQKPGQAPRLLIYGASRRATGIPD |
| | | RFSGSGSGTDFTLTISRLEPEDSAVYYCQQYHSSPSWTFGQGTKL |
| | | EIKTTTPAPRPPTPAPTIASQPLSLRPEACRPAAGGAVHTRGLDFA |
| | | CDIYIWAPLAGTCGVLLLSLVITLYCKRGRKKLLYIFKQPFMRPV |
| | | QTTQEEDGCSCRFPEEEEGGCELRVKFSRSADAPAYKQGQNQLY |
| | | NELNLGRREEYDVLDKRRGRDPEMGGKPRRKNPQEGLYNELQK |
| | | DKMAEAYSEIGMKGERRRGKGHDGLYQGLSTATKDTYDALHM |
| | | QALPPR |
| 139103-nt | 1411 | ATGGCCCTCCCTGTCACCGCCCTGCTGCTTCCGCTGGCTCTTC |
| Full CAR | | TGCTCCACGCCGCTCGGCCCCAAGTGCAACTCGTGGAATCTG |
| | | GTGGAGGACTCGTGCAACCCGGAAGATCGCTTAGACTGTCGT |
| | | GTGCCGCCAGCGGGTTCACTTTCTCGAACTACGCGATGTCCTG |
| | | GGTCCGCCAGGCACCCGGAAAGGGACTCGGTTGGGTGTCCGG |
| | | CATTTCCCGGTCCGGCGAAAATACCTACTACGCCGACTCCGTG |
| | | AAGGGCCGCTTCACCATCTCAAGGGACAACAGCAAAAACACC |
| | | CTGTACTTGCAAATGAACTCCCTGCGGGATGAAGATACAGCC |
| | | GTGTACTATTGCGCCCGGTCGCCTGCCCATTACTACGGCGGAA |
| | | TGGACGTCTGGGGACAGGGAACCACTGTGACTGTCAGCAGCG |
| | | CGTCGGGTGGCGGCGGCTCAGGGGGTCGGGCCTCCGGGGGGG |
| | | GAGGGTCCGACATCGTGCTGACCCAGTCCCCGGGAACCCTGA |
| | | GCCTGAGCCCGGGAGAGCGCGCGACCCTGTCATGCCGGGCAT |
| | | CCCAGAGCATTAGCTCCTCCTTTCTCGCCTGGTATCAGCAGAA |
| | | GCCCGGACAGGCCCCGAGGCTGCTGATCTACGGCGCTAGCAG |
| | | AAGGGCTACCGGAATCCCAGACCGGTTCTCCGGCTCCGGTTC |
| | | CGGGACCGATTTCACCCTTACTATCTCGCGCCTGGAACCTGAG |
| | | GACTCCGCCGTCTACTACTGCCAGCAGTACCACTCATCCCCGT |
| | | CGTGGACGTTCGGACAGGGCACCAAGCTGGAGATTAAGACCA |
| | | CTACCCCAGCACCGAGGCCACCCACCCCGGCTCCTACCATCG |
| | | CCTCCCAGCCTCTGTCCCTGCGTCCGGAGGCATGTAGACCCGC |
| | | AGCTGGTGGGGCCGTGCATACCCGGGGTCTTGACTTCGCCTG |
| | | CGATATCTACATTTGGGCCCCTCTGGCTGGTACTTGCGGGGTC |
| | | CTGCTGCTTTCACTCGTGATCACTCTTTACTGTAAGCGCGGTC |
| | | GGAAGAAGCTGCTGTACATCTTTAAGCAACCCTTCATGAGGC |
| | | CTGTGCAGACTACTCAAGAGGAGGACGGCTGTTCATGCCGGT |
| | | TCCCAGAGGAGGAGGAAGGCGGCTGCGAACTGCGCGTGAAA |
| | | TTCAGCCGCAGCGCAGATGCTCCAGCCTACAAGCAGGGGCAG |
| | | AACCAGCTCTACAACGAACTCAATCTTGGTCGGAGAGAGGAG |
| | | TACGACGTGCTGGACAAGCGGAGAGGACGGGACCCAGAAAT |
| | | GGGCGGGAAGCCGCGCAGAAAGAATCCCCAAGAGGGCCTGT |
| | | ACAACGAGCTCCAAAAGGATAAGATGGCAGAAGCCTATAGC |
| | | GAGATTGGTATGAAAGGGGAACGCAGAAGAGGCAAAGGCCA |
| | | CGACGGACTGTACCAGGGACTCAGCACCGCCACCAAGGACAC |
| | | CTATGACGCTCTTCACATGCAGGCCCTGCCGCCTCGG |

139105

| | | |
|---|---|---|
| 139105-aa | 1412 | QVQLVESGGGLVQPGRSLRLSCAASGFTFDDYAMHWVRQAPG |
| ScFv | | KGLEWVSGISWNSGSIGYADSVKGRFTISRDNAKNSLYLQMNSL |
| domain | | RAEDTALYYCSVHSFLAYWGQGTLVTVSSASGGGGSGGRASGG |
| | | GGSDIVMTQTPLSLPVTPGEPASISCRSSQSLLHSNGYNYLDWYL |
| | | QKPGQSPQLLIYLGSNRASGVPDRFSGSGSGTDFTLKISRVEAED |
| | | VGVYYCMQALQTPYTFGQGTKVEIK |
| 139105-nt | 1413 | CAAGTGCAACTCGTCGAATCCGGTGGAGGTCTGGTCCAACCT |
| ScFv | | GGTAGAAGCCTGAGACTGTCGTGTGCGGCCAGCGGATTCACC |
| domain | | TTTGATGACTATGCTATGCACTGGGTGCGGCAGGCCCCAGGA |
| | | AAGGGCCTGGAATGGGTGTCGGGAATTAGCTGGAACTCCGGG |
| | | TCCATTGGCTACGCCGACTCCGTGAAGGGCCGCTTCACCATCT |
| | | CCCGCGACAACGCAAAGAACTCCCTGTACTTGCAAATGAACT |
| | | CGCTCAGGGCTGAGGATACCGCGCTGTACTACTGCTCCGTGC |
| | | ATTCCTTCCTGGCCTACTGGGGACAGGGAACTCTGGTCACCGT |
| | | GTCGAGCGCCTCCGGCGGCGGGGGCTCGGGTGGACGGGCCTC |
| | | GGGCGGAGGGGGGTCCGACATCGTGATGACCCAGACCCCGCT |
| | | GAGCTTGCCCGTGACTCCCGGAGAGCCTGCATCCATCTCCTGC |
| | | CGGTCATCCCAGTCCCTTCTCCACTCCAACGGATACAACTACC |
| | | TCGACTGGTACCTCCAGAAGCCGGGACAGAGCCCTCAGCTTC |
| | | TGATCTACCTGGGGTCAAATAGAGCCTCAGGAGTGCCGGATC |
| | | GGTTCAGCGGATCTGGTTCGGGAACTGATTTCACTCTGAAGAT |
| | | TTCCCGCGTGGAAGCCGAGGACGTGGGCGTCTACTACTGTAT |
| | | GCAGGCGCTGCAGACCCCCTATACCTTCGGCCAAGGGACGAA |
| | | AGTGGAGATCAAG |
| 139105-aa | 1414 | QVQLVESGGGLVQPGRSLRLSCAASGFTFDDYAMHWVRQAPG |
| VH | | KGLEWVSGISWNSGSIGYADSVKGRFTISRDNAKNSLYLQMNSL |
| | | RAEDTALYYCSVHSFLAYWGQGTLVTVSS |
| 139105-aa | 1415 | DIVMTQTPLSLPVTPGEPASISCRSSQSLLHSNGYNYLDWYLQKP |
| VL | | GQSPQLLIYLGSNRASGVPDRFSGSGSGTDFTLKISRVEAEDVGV |
| | | YYCMQALQTPYTFGQGTKVEIK |
| 139105-aa | 1416 | MALPVTALLLPLALLLHAARPQVQLVESGGGLVQPGRSLRLSCA |
| Full CAR | | ASGFTFDDYAMHWVRQAPGKGLEWVSGISWNSGSIGYADSVK |
| | | GRFTISRDNAKNSLYLQMNSLRAEDTALYYCSVHSFLAYWGQG |
| | | TLVTVSSASGGGGSGGRASGGGGSDIVMTQTPLSLPVTPGEPASI |
| | | SCRSSQSLLHSNGYNYLDWYLQKPGQSPQLLIYLGSNRASGVPD |
| | | RFSGSGSGTDFTLKISRVEAEDVGVYYCMQALQTPYTFGQGTKV |
| | | EIKTTTPAPRPPTPAPTIASQPLSLRPEACRPAAGGAVHTRGLDFA |
| | | CDIYIWAPLAGTCGVLLLSLVITLYCKRGRKKLLYIFKQPFMRPV |
| | | QTTQEEDGCSCRFPEEEEGGCELRVKFSRSADAPAYKQGQNQLY |
| | | NELNLGRREEYDVLDKRRGRDPEMGGKPRRKNPQEGLYNELQK |
| | | DKMAEAYSEIGMKGERRRGKGHDGLYQGLSTATKDTYDALHM |
| | | QALPPR |
| 139105-nt | 1417 | ATGGCCCTCCCTGTCACCGCCCTGCTGCTTCCGCTGGCTCTTC |
| Full CAR | | TGCTCCACGCCGCTCGGCCCCAAGTGCAACTCGTCGAATCCG |
| | | GTGGAGGTCTGGTCCAACCTGGTAGAAGCCTGAGACTGTCGT |
| | | GTGCGGCCAGCGGATTCACCTTTGATGACTATGCTATGCACTG |
| | | GGTGCGGCAGGCCCCAGGAAAGGGCCTGGAATGGGTGTCGG |
| | | GAATTAGCTGGAACTCCGGGTCCATTGGCTACGCCGACTCCG |
| | | TGAAGGGCCGCTTCACCATCTCCCGCGACAACGCAAAGAACT |
| | | CCCTGTACTTGCAAATGAACTCGCTCAGGGCTGAGGATACCG |
| | | CGCTGTACTACTGCTCCGTGCATTCCTTCCTGGCCTACTGGGG |
| | | ACAGGGAACTCTGGTCACCGTGTCGAGCGCCTCCGGCGGCGG |
| | | GGGCTCGGGTGGACGGGCCTCGGGCGGAGGGGGGTCCGACA |
| | | TCGTGATGACCCAGACCCCGCTGAGCTTGCCCGTGACTCCCG |
| | | GAGAGCCTGCATCCATCTCCTGCCGGTCATCCCAGTCCCTTCT |
| | | CCACTCCAACGGATACAACTACCTCGACTGGTACCTCCAGAA |
| | | GCCGGGACAGAGCCCTCAGCTTCTGATCTACCTGGGGTCAAA |
| | | TAGAGCCTCAGGAGTGCCGGATCGGTTCAGCGGATCTGGTTC |
| | | GGGAACTGATTTCACTCTGAAGATTTCCCGCGTGGAAGCCGA |
| | | GGACGTGGGCGTCTACTACTGTATGCAGGCGCTGCAGACCCC |
| | | CTATACCTTCGGCCAAGGGACGAAAGTGGAGATCAAGACCAC |
| | | TACCCCAGCACCGAGGCCACCCACCCCGGCTCCTACCATCGC |
| | | CTCCCAGCCTCTGTCCCTGCGTCCGGAGGCATGTAGACCCGCA |
| | | GCTGGTGGGGCCGTGCATACCCGGGGTCTTGACTTCGCCTGC |
| | | GATATCTACATTTGGGCCCCTCTGGCTGGTACTTGCGGGGTCC |
| | | TGCTGCTTTCACTCGTGATCACTCTTTACTGTAAGCGCGGTCG |
| | | GAAGAAGCTGCTGTACATCTTTAAGCAACCCTTCATGAGGCC |
| | | TGTGCAGACTACTCAAGAGGAGGACGGCTGTTCATGCCGGTT |
| | | CCCAGAGGAGGAGGAAGGCGGCTGCGAACTGCGCGTGAAAT |
| | | TCAGCCGCAGCGCAGATGCTCCAGCCTACAAGCAGGGGCAGA |
| | | ACCAGCTCTACAACGAACTCAATCTTGGTCGGAGAGAGGAGT |
| | | ACGACGTGCTGGACAAGCGGAGAGGACGGGACCCAGAAATG |
| | | GGCGGGAAGCCGCGCAGAAAGAATCCCCAAGAGGGCCTGTA |
| | | CAACGAGCTCCAAAAGGATAAGATGGCAGAAGCCTATAGCG |
| | | AGATTGGTATGAAAGGGGAACGCAGAAGAGGCAAAGGCCAC |
| | | GACGGACTGTACCAGGGACTCAGCACCGCCACCAAGGACACC |
| | | TATGACGCTCTTCACATGCAGGCCCTGCCGCCTCGG |

139111

| | | |
|---|---|---|
| 139111-aa | 1418 | EVQLLESGGGLVQPGGSLRLSCAVSGFALSNHGMSWVRRAPGK |
| ScFv | | GLEWVSGIVYSGSTYYAASVKGRFTISRDNSRNTLYLQMNSLRP |
| domain | | EDTAIYYCSAHGGESDVWGQGTTVTVSSASGGGGSGGRASGGG |
| | | GSDIVMTQTPLSLSVTPGQPASISCKSSQSLLRNDGKTPLYWYLQ |
| | | KAGQPPQLLIYEVSNRFSGVPDRFSGSGSGTDFTLKISRVEAEDV |
| | | GAYYCMQNIQFPSFGGGTKLEIK |
| 139111-nt | 1419 | GAAGTGCAATTGTTGGAATCTGGAGGAGGACTTGTGCAGCCT |
| ScFv | | GGAGGATCACTGAGACTTTCGTGTGCGGTGTCAGGCTTCGCC |
| domain | | CTGAGCAACCACGGCATGAGCTGGGTGCGGAGAGCCCCGGG |
| | | GAAGGGTCTGGAATGGGTGTCCGGGATCGTCTACTCCGGTTC |
| | | AACTTACTACGCCGCAAGCGTGAAGGGTCGCTTCACCATTTCC |
| | | CGCGATAACTCCCGGAACACCCTGTACCTCCAAATGAACTCC |
| | | CTGCGGCCCGAGGACACCGCCATCTACTACTGTTCCGCGCAT |
| | | GGAGGAGAGTCCGATGTCTGGGGACAGGGCACTACCGTGACC |
| | | GTGTCGAGCGCCTCGGGGGGAGGAGGCTCCGGCGGTCGCGCC |
| | | TCCGGGGGGGGTGGCAGCGACATTGTGATGACGCAGACTCCA |
| | | CTCTCGCTGTCCGTGACCCCGGGACAGCCCGCGTCCATCTCGT |
| | | GCAAGAGCTCCCAGAGCCTGCTGAGGAACGACGGAAAGACT |
| | | CCTCTGTATTGGTACCTCCAGAAGGCTGGACAGCCCCCGCAA |
| | | CTGCTCATCTACGAAGTGTCAAATCGCTTCTCCGGGGTGCCGG |
| | | ATCGGTTTTCCGGCTCGGGATCGGGCACCGACTTCACCCTGAA |
| | | AATCTCCAGGGTCGAGGCCGAGGACGTGGGAGCCTACTACTG |
| | | CATGCAAAACATCCAGTTCCCTTCCTTCGGCGGCGGCACAAA |
| | | GCTGGAGATTAAG |
| 139111-aa | 1420 | EVQLLESGGGLVQPGGSLRLSCAVSGFALSNHGMSWVRRAPGK |
| VH | | GLEWVSGIVYSGSTYYAASVKGRFTISRDNSRNTLYLQMNSLRP |
| | | EDTAIYYCSAHGGESDVWGQGTTVTVSS |
| 139111-aa | 1421 | DIVMTQTPLSLSVTPGQPASISCKSSQSLLRNDGKTPLYWYLQKA |
| VL | | GQPPQLLIYEVSNRFSGVPDRFSGSGSGTDFTLKISRVEAEDVGA |
| | | YYCMQNIQFPSFGGGTKLEIK |
| 139111-aa | 1422 | MALPVTALLLPLALLLHAARPEVQLLESGGGLVQPGGSLRLSCA |
| Full CAR | | VSGFALSNHGMSWVRRAPGKGLEWVSGIVYSGSTYYAASVKGR |
| | | FTISRDNSRNTLYLQMNSLRPEDTAIYYCSAHGGESDVWGQGTT |
| | | VTVSSASGGGGSGGRASGGGGSDIVMTQTPLSLSVTPGQPASISC |
| | | KSSQSLLRNDGKTPLYWYLQKAGQPPQLLIYEVSNRFSGVPDRF |
| | | SGSGSGTDFTLKISRVEAEDVGAYYCMQNIQFPSFGGGTKLEIKT |
| | | TTPAPRPPTPAPTIASQPLSLRPEACRPAAGGAVHTRGLDFACDIY |
| | | IWAPLAGTCGVLLLSLVITLYCKRGRKKLLYIFKQPFMRPVQTTQ |
| | | EEDGCSCRFPEEEEGGCELRVKFSRSADAPAYKQGQNQLYNELN |
| | | LGRREEYDVLDKRRGRDPEMGGKPRRKNPQEGLYNELQKDKM |
| | | AEAYSEIGMKGERRRGKGHDGLYQGLSTATKDTYDALHMQAL |
| | | PPR |
| 139111-nt | 1423 | ATGGCCCTCCCTGTCACCGCCCTGCTGCTTCCGCTGGCTCTTC |
| Full CAR | | TGCTCCACGCCGCTCGGCCCGAAGTGCAATTGTTGGAATCTG |
| | | GAGGAGGACTTGTGCAGCCTGGAGGATCACTGAGACTTTCGT |
| | | GTGCGGTGTCAGGCTTCGCCCTGAGCAACCACGGCATGAGCT |
| | | GGGTGCGGAGAGCCCCGGGGAAGGGTCTGGAATGGGTGTCC |
| | | GGGATCGTCTACTCCGGTTCAACTTACTACGCCGCAAGCGTG |
| | | AAGGGTCGCTTCACCATTTCCCGCGATAACTCCCGGAACACC |
| | | CTGTACCTCCAAATGAACTCCCTGCGGCCCGAGGACACCGCC |
| | | ATCTACTACTGTTCCGCGCATGGAGGAGAGTCCGATGTCTGG |
| | | GGACAGGGCACTACCGTGACCGTGTCGAGCGCCTCGGGGGGA |
| | | GGAGGCTCCGGCGGTCGCGCCTCCGGGGGGGGTGGCAGCGAC |
| | | ATTGTGATGACGCAGACTCCACTCTCGCTGTCCGTGACCCCGG |
| | | GACAGCCCGCGTCCATCTCGTGCAAGAGCTCCCAGAGCCTGC |
| | | TGAGGAACGACGGAAAGACTCCTCTGTATTGGTACCTCCAGA |
| | | AGGCTGGACAGCCCCCGCAACTGCTCATCTACGAAGTGTCAA |
| | | ATCGCTTCTCCGGGGTGCCGGATCGGTTTTCCGGCTCGGGATC |
| | | GGGCACCGACTTCACCCTGAAAATCTCCAGGGTCGAGGCCGA |
| | | GGACGTGGGAGCCTACTACTGCATGCAAAACATCCAGTTCCC |
| | | TTCCTTCGGCGGCGGCACAAAGCTGGAGATTAAGACCACTAC |
| | | CCCAGCACCGAGGCCACCCACCCCGGCTCCTACCATCGCCTC |
| | | CCAGCCTCTGTCCCTGCGTCCGGAGGCATGTAGACCCGCAGC |
| | | TGGTGGGGCCGTGCATACCCGGGGTCTTGACTTCGCCTGCGAT |
| | | ATCTACATTTGGGCCCCTCTGGCTGGTACTTGCGGGGTCCTGC |
| | | TGCTTTCACTCGTGATCACTCTTTACTGTAAGCGCGGTCGGAA |
| | | GAAGCTGCTGTACATCTTTAAGCAACCCTTCATGAGGCCTGTG |
| | | CAGACTACTCAAGAGGAGGACGGCTGTTCATGCCGGTTCCCA |
| | | GAGGAGGAGGAAGGCGGCTGCGAACTGCGCGTGAAATTCAG |
| | | CCGCAGCGCAGATGCTCCAGCCTACAAGCAGGGGCAGAACCA |
| | | GCTCTACAACGAACTCAATCTTGGTCGGAGAGAGGAGTACGA |
| | | CGTGCTGGACAAGCGGAGAGGACGGGACCCAGAAATGGGCG |
| | | GGAAGCCGCGCAGAAAGAATCCCCAAGAGGGCCTGTACAAC |
| | | GAGCTCCAAAAGGATAAGATGGCAGAAGCCTATAGCGAGATT |
| | | GGTATGAAAGGGGAACGCAGAAGAGGCAAAGGCCACGACGG |
| | | ACTGTACCAGGGACTCAGCACCGCCACCAAGGACACCTATGA |
| | | CGCTCTTCACATGCAGGCCCTGCCGCCTCGG |

139100

| | | |
|---|---|---|
| 139100-aa | 1424 | QVQLVQSGAEVRKTGASVKVSCKASGYIFDNFGINWVRQAPGQ |
| ScFv | | GLEWMGWINPKNNNTNYAQKFQGRVTITADESTNTAYMEVSSL |
| domain | | RSEDTAVYYCARGPYYYQSYMDVWGQGTMVTVSSASGGGGSG |
| | | GRASGGGGSDIVMTQTPLSLPVTPGEPASISCRSSQSLLHSNGYN |
| | | YLNWYLQKPGQSPQLLIYLGSKRASGVPDRFSGSGSGTDFTLHIT |
| | | RVGAEDVGVYYCMQALQTPYTFGQGTKLEIK |
| 139100-nt | 1425 | CAAGTCCAACTCGTCCAGTCCGGCGCAGAAGTCAGAAAAACC |
| ScFv | | GGTGCTAGCGTGAAAGTGTCCTGCAAGGCCTCCGGCTACATT |
| domain | | TTCGATAACTTCGGAATCAACTGGGTCAGACAGGCCCCGGGC |
| | | CAGGGGCTGGAATGGATGGGATGGATCAACCCCAAGAACAA |
| | | CAACACCAACTACGCACAGAAGTTCCAGGGCCGCGTGACTAT |
| | | CACCGCCGATGAATCGACCAATACCGCCTACATGGAGGTGTC |
| | | CTCCCTGCGGTCGGAGGACACTGCCGTGTATTACTGCGCGAG |
| | | GGGCCCATACTACTACCAAAGCTACATGGACGTCTGGGGACA |
| | | GGGAACCATGGTGACCGTGTCATCCGCCTCCGGTGGTGGAGG |
| | | CTCCGGGGGGCGGGCTTCAGGAGGCGGAGGAAGCGATATTGT |
| | | GATGACCCAGACTCCGCTTAGCCTGCCCGTGACTCCTGGAGA |
| | | ACCGGCCTCCATTTCCTGCCGGTCCTCGCAATCACTCCTGCAT |
| | | TCCAACGGTTACAACTACCTGAATTGGTACCTCCAGAAGCCT |
| | | GGCCAGTCGCCCCAGTTGCTGATCTATCTGGGCTCGAAGCGC |
| | | GCCTCCGGGGTGCCTGACCGGTTTAGCGGATCTGGGAGCGGC |
| | | ACGGACTTCACTCTCCACATCACCCGCGTGGGAGCGGAGGAC |
| | | GTGGGAGTGTACTACTGTATGCAGGCGCTGCAGACTCCGTAC |
| | | ACATTCGGACAGGGCACCAAGCTGGAGATCAAG |
| 139100-aa | 1426 | QVQLVQSGAEVRKTGASVKVSCKASGYIFDNFGINWVRQAPGQ |
| VH | | GLEWMGWINPKNNNTNYAQKFQGRVTITADESTNTAYMEVSSL |
| | | RSEDTAVYYCARGPYYYQSYMDVWGQGTMVTVSS |
| 139100-aa | 1427 | DIVMTQTPLSLPVTPGEPASISCRSSQSLLHSNGYNYLNWYLQKP |
| VL | | GQSPQLLIYLGSKRASGVPDRFSGSGSGTDFTLHITRVGAEDVGV |
| | | YYCMQALQTPYTFGQGTKLEIK |
| 139100-aa | 1428 | MALPVTALLLPLALLLHAARPQVQLVQSGAEVRKTGASVKVSC |
| Full CAR | | KASGYIFDNFGINWVRQAPGQGLEWMGWINPKNNNTNYAQKF |
| | | QGRVTITADESTNTAYMEVSSLRSEDTAVYYCARGPYYYQSYM |
| | | DVWGQGTMVTVSSASGGGGSGGRASGGGGSDIVMTQTPLSLPV |
| | | TPGEPASISCRSSQSLLHSNGYNYLNWYLQKPGQSPQLLIYLGSK |
| | | RASGVPDRFSGSGSGTDFTLHITRVGAEDVGVYYCMQALQTPYT |
| | | FGQGTKLEIKTTTPAPRPPTPAPTIASQPLSLRPEACRPAAGGAVH |
| | | TRGLDFACDIYIWAPLAGTCGVLLLSLVITLYCKRGRKKLLYIFK |
| | | QPFMRPVQTTQEEDGCSCRFPEEEEGGCELRVKFSRSADAPAYK |
| | | QGQNQLYNELNLGRREEYDVLDKRRGRDPEMGGKPRRKNPQE |
| | | GLYNELQKDKMAEAYSEIGMKGERRRGKGHDGLYQGLSTATK |
| | | DTYDALHMQALPPR |
| 139100-nt | 1429 | ATGGCCCTCCCTGTCACCGCCCTGCTGCTTCCGCTGGCTCTTC |
| Full CAR | | TGCTCCACGCCGCTCGGCCCCAAGTCCAACTCGTCCAGTCCGG |
| | | CGCAGAAGTCAGAAAAACCGGTGCTAGCGTGAAAGTGTCCTG |
| | | CAAGGCCTCCGGCTACATTTTCGATAACTTCGGAATCAACTGG |
| | | GTCAGACAGGCCCCGGGCCAGGGGCTGGAATGGATGGGATG |
| | | GATCAACCCCAAGAACAACAACACCAACTACGCACAGAAGTT |
| | | CCAGGGCCGCGTGACTATCACCGCCGATGAATCGACCAATAC |
| | | CGCCTACATGGAGGTGTCCTCCCTGCGGTCGGAGGACACTGC |
| | | CGTGTATTACTGCGCGAGGGGCCCATACTACTACCAAAGCTA |
| | | CATGGACGTCTGGGGACAGGGAACCATGGTGACCGTGTCATC |
| | | CGCCTCCGGTGGTGGAGGCTCCGGGGGGCGGGCTTCAGGAGG |
| | | CGGAGGAAGCGATATTGTGATGACCCAGACTCCGCTTAGCCT |
| | | GCCCGTGACTCCTGGAGAACCGGCCTCCATTTCCTGCCGGTCC |
| | | TCGCAATCACTCCTGCATTCCAACGGTTACAACTACCTGAATT |
| | | GGTACCTCCAGAAGCCTGGCCAGTCGCCCCAGTTGCTGATCT |
| | | ATCTGGGCTCGAAGCGCGCCTCCGGGGTGCCTGACCGGTTTA |
| | | GCGGATCTGGGAGCGGCACGGACTTCACTCTCCACATCACCC |
| | | GCGTGGGAGCGGAGGACGTGGGAGTGTACTACTGTATGCAGG |
| | | CGCTGCAGACTCCGTACACATTCGGACAGGGCACCAAGCTGG |
| | | AGATCAAGACCACTACCCCAGCACCGAGGCCACCCACCCCGG |
| | | CTCCTACCATCGCCTCCCAGCCTCTGTCCCTGCGTCCGGAGGC |
| | | ATGTAGACCCGCAGCTGGTGGGGCCGTGCATACCCGGGGTCT |
| | | TGACTTCGCCTGCGATATCTACATTTGGGCCCCTCTGGCTGGT |
| | | ACTTGCGGGGTCCTGCTGCTTTCACTCGTGATCACTCTTTACT |
| | | GTAAGCGCGGTCGGAAGAAGCTGCTGTACATCTTTAAGCAAC |
| | | CCTTCATGAGGCCTGTGCAGACTACTCAAGAGGAGGACGGCT |
| | | GTTCATGCCGGTTCCCAGAGGAGGAGGAAGGCGGCTGCGAAC |
| | | TGCGCGTGAAATTCAGCCGCAGCGCAGATGCTCCAGCCTACA |
| | | AGCAGGGGCAGAACCAGCTCTACAACGAACTCAATCTTGGTC |
| | | GGAGAGAGGAGTACGACGTGCTGGACAAGCGGAGAGGACGG |
| | | GACCCAGAAATGGGCGGGAAGCCGCGCAGAAAGAATCCCCA |
| | | AGAGGGCCTGTACAACGAGCTCCAAAAGGATAAGATGGCAG |
| | | AAGCCTATAGCGAGATTGGTATGAAAGGGGAACGCAGAAGA |
| | | GGCAAAGGCCACGACGGACTGTACCAGGGACTCAGCACCGCC |
| | | ACCAAGGACACCTATGACGCTCTTCACATGCAGGCCCTGCCG |
| | | CCTCGG |

139101

| | | |
|---|---|---|
| 139101-aa | 1430 | QVQLQESGGGLVQPGGSLRLSCAASGFTFSSDAMTWVRQAPGK |
| ScFv | | GLEWVSVISGSGGTTYYADSVKGRFTISRDNSKNTLYLQMNSLR |
| domain | | AEDTAVYYCAKLDSSGYYYARGPRYWGQGTLVTVSSASGGGG |
| | | SGGRASGGGGSDIQLTQSPSSLSASVGDRVTITCRASQSISSYLN |
| | | WYQQKPGKAPKLLIYGASTLASGVPARFSGSGSGTHFTLTINSLQ |
| | | SEDSATYYCQQSYKRASFGQGTKVEIK |
| 139101-nt | 1431 | CAAGTGCAACTTCAAGAATCAGGCGGAGGACTCGTGCAGCCC |
| ScFv | | GGAGGATCATTGCGGCTCTCGTGCGCCGCCTCGGGCTTCACCT |
| domain | | TCTCGAGCGACGCCATGACCTGGGTCCGCCAGGCCCCGGGGA |
| | | AGGGGCTGGAATGGGTGTCTGTGATTTCCGGCTCCGGGGGAA |
| | | CTACGTACTACGCCGATTCCGTGAAAGGTCGCTTCACTATCTC |
| | | CCGGGACAACAGCAAGAACACCCTTTATCTGCAAATGAATTC |
| | | CCTCCGCGCCGAGGACACCGCCGTGTACTACTGCGCCAAGCT |
| | | GGACTCCTCGGGCTACTACTATGCCCGGGGTCCGAGATACTG |
| | | GGGACAGGGAACCCTCGTGACCGTGTCCTCCGCGTCCGGCGG |
| | | AGGAGGGTCGGGAGGGCGGGCCTCCGGCGGCGGCGGTTCGG |
| | | ACATCCAGCTGACCCAGTCCCCATCCTCACTGAGCGCAAGCG |
| | | TGGGCGACAGAGTCACCATTACATGCAGGGCGTCCCAGAGCA |
| | | TCAGCTCCTACCTGAACTGGTACCAACAGAAGCCTGGAAAGG |
| | | CTCCTAAGCTGTTGATCTACGGGGCTTCGACCCTGGCATCCGG |
| | | GGTGCCCGCGAGGTTTAGCGGAAGCGGTAGCGGCACTCACTT |
| | | CACTCTGACCATTAACAGCCTCCAGTCCGAGGATTCAGCCACT |
| | | TACTACTGTCAGCAGTCCTACAAGCGGGCCAGCTTCGGACAG |
| | | GGCACTAAGGTCGAGATCAAG |
| 139101-aa | 1432 | QVQLQESGGGLVQPGGSLRLSCAASGFTFSSDAMTWVRQAPGK |
| VH | | GLEWVSVISGSGGTTYYADSVKGRFTISRDNSKNTLYLQMNSLR |
| | | AEDTAVYYCAKLDSSGYYYARGPRYWGQGTLVTVSS |
| 139101-aa | 1433 | DIQLTQSPSSLSASVGDRVTITCRASQSISSYLNWYQQKPGKAPK |
| VL | | LLIYGASTLASGVPARFSGSGSGTHFTLTINSLQSEDSATYYCQQS |
| | | YKRASFGQGTKVEIK |
| 139101-aa | 1434 | MALPVTALLLPLALLLHAARPQVQLQESGGGLVQPGGSLRLSCA |
| Full CAR | | ASGFTFSSDAMTWVRQAPGKGLEWVSVISGSGGTTYYADSVKG |
| | | RFTISRDNSKNTLYLQMNSLRAEDTAVYYCAKLDSSGYYYARG |
| | | PRYWGQGTLVTVSSASGGGGSGGRASGGGGSDIQLTQSPSSLSA |
| | | SVGDRVTITCRASQSISSYLNWYQQKPGKAPKLLIYGASTLASGV |
| | | PARFSGSGSGTHFTLTINSLQSEDSATYYCQQSYKRASFGQGTKV |
| | | EIKTTTPAPRPPTPAPTIASQPLSLRPEACRPAAGGAVHTRGLDFA |
| | | CDIYIWAPLAGTCGVLLLSLVITLYCKRGRKKLLYIFKQPFMRPV |
| | | QTTQEEDGCSCRFPEEEEGGCELRVKFSRSADAPAYKQGQNQLY |
| | | NELNLGRREEYDVLDKRRGRDPEMGGKPRRKNPQEGLYNELQK |
| | | DKMAEAYSEIGMKGERRRGKGHDGLYQGLSTATKDTYDALHM |
| | | QALPPR |
| 139101-nt | 1435 | ATGGCCCTCCCTGTCACCGCCCTGCTGCTTCCGCTGGCTCTTC |
| Full CAR | | TGCTCCACGCCGCTCGGCCCCAAGTGCAACTTCAAGAATCAG |
| | | GCGGAGGACTCGTGCAGCCCGGAGGATCATTGCGGCTCTCGT |
| | | GCGCCGCCTCGGGCTTCACCTTCTCGAGCGACGCCATGACCTG |
| | | GGTCCGCCAGGCCCCGGGGAAGGGGCTGGAATGGGTGTCTGT |
| | | GATTTCCGGCTCCGGGGGAACTACGTACTACGCCGATTCCGT |
| | | GAAAGGTCGCTTCACTATCTCCCGGGACAACAGCAAGAACAC |
| | | CCTTTATCTGCAAATGAATTCCCTCCGCGCCGAGGACACCGCC |
| | | GTGTACTACTGCGCCAAGCTGGACTCCTCGGGCTACTACTATG |
| | | CCCGGGGTCCGAGATACTGGGGACAGGGAACCCTCGTGACCG |
| | | TGTCCTCCGCGTCCGGCGGAGGAGGGTCGGGAGGGCGGGCCT |
| | | CCGGCGGCGGCGGTTCGGACATCCAGCTGACCCAGTCCCCAT |
| | | CCTCACTGAGCGCAAGCGTGGGCGACAGAGTCACCATTACAT |
| | | GCAGGGCGTCCCAGAGCATCAGCTCCTACCTGAACTGGTACC |
| | | AACAGAAGCCTGGAAAGGCTCCTAAGCTGTTGATCTACGGGG |
| | | CTTCGACCCTGGCATCCGGGGTGCCCGCGAGGTTTAGCGGAA |
| | | GCGGTAGCGGCACTCACTTCACTCTGACCATTAACAGCCTCCA |
| | | GTCCGAGGATTCAGCCACTTACTACTGTCAGCAGTCCTACAA |
| | | GCGGGCCAGCTTCGGACAGGGCACTAAGGTCGAGATCAAGAC |
| | | CACTACCCCAGCACCGAGGCCACCCACCCCGGCTCCTACCAT |
| | | CGCCTCCCAGCCTCTGTCCCTGCGTCCGGAGGCATGTAGACCC |
| | | GCAGCTGGTGGGGCCGTGCATACCCGGGGTCTTGACTTCGCC |
| | | TGCGATATCTACATTTGGGCCCCTCTGGCTGGTACTTGCGGGG |
| | | TCCTGCTGCTTTCACTCGTGATCACTCTTTACTGTAAGCGCGG |
| | | TCGGAAGAAGCTGCTGTACATCTTTAAGCAACCCTTCATGAG |
| | | GCCTGTGCAGACTACTCAAGAGGAGGACGGCTGTTCATGCCG |
| | | GTTCCCAGAGGAGGAGGAAGGCGGCTGCGAACTGCGCGTGA |
| | | AATTCAGCCGCAGCGCAGATGCTCCAGCCTACAAGCAGGGGC |
| | | AGAACCAGCTCTACAACGAACTCAATCTTGGTCGGAGAGAGG |
| | | AGTACGACGTGCTGGACAAGCGGAGAGGACGGGACCCAGAA |
| | | ATGGGCGGGAAGCCGCGCAGAAAGAATCCCCAAGAGGGCCT |
| | | GTACAACGAGCTCCAAAAGGATAAGATGGCAGAAGCCTATA |
| | | GCGAGATTGGTATGAAAGGGGAACGCAGAAGAGGCAAAGGC |
| | | CACGACGGACTGTACCAGGGACTCAGCACCGCCACCAAGGAC |
| | | ACCTATGACGCTCTTCACATGCAGGCCCTGCCGCCTCGG |

139102

| | | |
|---|---|---|
| 139102-aa | 1436 | QVQLVQSGAEVKKPGASVKVSCKASGYTFSNYGITWVRQAPGQ |
| ScFv | | GLEWMGWISAYNGNTNYAQKFQGRVTMTRNTSISTAYMELSSL |
| domain | | RSEDTAVYYCARGPYYYYMDVWGKGTMVTVSSASGGGGSGG |
| | | RASGGGGSEIVMTQSPLSLPVTPGEPASISCRSSQSLLYSNGYNY |
| | | VDWYLQKPGQSPQLLIYLGSNRASGVPDRFSGSGSGTDFKLQISR |
| | | VEAEDVGIYYCMQGRQFPYSFGQGTKVEIK |
| 139102-nt | 1437 | CAAGTCCAACTGGTCCAGAGCGGTGCAGAAGTGAAGAAGCCC |
| ScFv | | GGAGCGAGCGTGAAAGTGTCCTGCAAGGCTTCCGGGTACACC |
| domain | | TTCTCCAACTACGGCATCACTTGGGTGCGCCAGGCCCCGGGA |
| | | CAGGGCCTGGAATGGATGGGGTGGATTTCCGCGTACAACGGC |
| | | AATACGAACTACGCTCAGAAGTTCCAGGGTAGAGTGACCATG |
| | | ACTAGGAACACCTCCATTTCCACCGCCTACATGGAACTGTCCT |
| | | CCCTGCGGAGCGAGGACACCGCCGTGTACTATTGCGCCCGGG |
| | | GACCATACTACTACTACATGGATGTCTGGGGGAAGGGGACTA |
| | | TGGTCACCGTGTCATCCGCCTCGGGAGGCGGCGGATCAGGAG |
| | | GACGCGCCTCTGGTGGTGGAGGATCGGAGATCGTGATGACCC |
| | | AGAGCCCTCTCTCCTTGCCCGTGACTCCTGGGGAGCCCGCATC |
| | | CATTTCATGCCGGAGCTCCCAGTCACTTCTCTACTCCAACGGC |
| | | TATAACTACGTGGATTGGTACCTCCAAAAGCCGGGCCAGAGC |
| | | CCGCAGCTGCTGATCTACCTGGGCTCGAACAGGGCCAGCGGA |
| | | GTGCCTGACCGGTTCTCCGGGTCGGGAAGCGGGACCGACTTC |
| | | AAGCTGCAAATCTCGAGAGTGGAGGCCGAGGACGTGGGAAT |
| | | CTACTACTGTATGCAGGGCCGCCAGTTTCCGTACTCGTTCGGA |
| | | CAGGGCACCAAAGTGGAAATCAAG |
| 139102-aa | 1438 | QVQLVQSGAEVKKPGASVKVSCKASGYTFSNYGITWVRQAPGQ |
| VH | | GLEWMGWISAYNGNTNYAQKFQGRVTMTRNTSISTAYMELSSL |
| | | RSEDTAVYYCARGPYYYYMDVWGKGTMVTVSS |
| 139102-aa | 1439 | EIVMTQSPLSLPVTPGEPASISCRSSQSLLYSNGYNYVDWYLQKP |
| VL | | GQSPQLLIYLGSNRASGVPDRFSGSGSGTDFKLQISRVEAEDVGI |
| | | YYCMQGRQFPYSFGQGTKVEIK |
| 139102-aa | 1440 | MALPVTALLLPLALLLHAARPQVQLVQSGAEVKKPGASVKVSC |
| Full CAR | | KASGYTFSNYGITWVRQAPGQGLEWMGWISAYNGNTNYAQKF |
| | | QGRVTMTRNTSISTAYMELSSLRSEDTAVYYCARGPYYYYMDV |
| | | WGKGTMVTVSSASGGGGSGGRASGGGGSEIVMTQSPLSLPVTP |
| | | GEPASISCRSSQSLLYSNGYNYVDWYLQKPGQSPQLLIYLGSNRA |
| | | SGVPDRFSGSGSGTDFKLQISRVEAEDVGIYYCMQGRQFPYSFG |
| | | QGTKVEIKTTTPAPRPPTPAPTIASQPLSLRPEACRPAAGGAVHTR |
| | | GLDFACDIYIWAPLAGTCGVLLLSLVITLYCKRGRKKLLYIFKQP |
| | | FMRPVQTTQEEDGCSCRFPEEEEGGCELRVKFSRSADAPAYKQG |
| | | QNQLYNELNLGRREEYDVLDKRRGRDPEMGGKPRRKNPQEGL |
| | | YNELQKDKMAEAYSEIGMKGERRRGKGHDGLYQGLSTATKDT |
| | | YDALHMQALPPR |
| 139102-nt | 1441 | ATGGCCCTCCCTGTCACCGCCCTGCTGCTTCCGCTGGCTCTTC |
| Full CAR | | TGCTCCACGCCGCTCGGCCCCAAGTCCAACTGGTCCAGAGCG |
| | | GTGCAGAAGTGAAGAAGCCCGGAGCGAGCGTGAAAGTGTCC |
| | | TGCAAGGCTTCCGGGTACACCTTCTCCAACTACGGCATCACTT |
| | | GGGTGCGCCAGGCCCCGGGACAGGGCCTGGAATGGATGGGG |
| | | TGGATTTCCGCGTACAACGGCAATACGAACTACGCTCAGAAG |
| | | TTCCAGGGTAGAGTGACCATGACTAGGAACACCTCCATTTCC |
| | | ACCGCCTACATGGAACTGTCCTCCCTGCGGAGCGAGGACACC |
| | | GCCGTGTACTATTGCGCCCGGGGACCATACTACTACTACATG |
| | | GATGTCTGGGGGAAGGGGACTATGGTCACCGTGTCATCCGCC |
| | | TCGGGAGGCGGCGGATCAGGAGGACGCGCCTCTGGTGGTGGA |
| | | GGATCGGAGATCGTGATGACCCAGAGCCCTCTCTCCTTGCCC |
| | | GTGACTCCTGGGGAGCCCGCATCCATTTCATGCCGGAGCTCCC |
| | | AGTCACTTCTCTACTCCAACGGCTATAACTACGTGGATTGGTA |
| | | CCTCCAAAAGCCGGGCCAGAGCCCGCAGCTGCTGATCTACCT |
| | | GGGCTCGAACAGGGCCAGCGGAGTGCCTGACCGGTTCTCCGG |
| | | GTCGGGAAGCGGGACCGACTTCAAGCTGCAAATCTCGAGAGT |
| | | GGAGGCCGAGGACGTGGGAATCTACTACTGTATGCAGGGCCG |
| | | CCAGTTTCCGTACTCGTTCGGACAGGGCACCAAAGTGGAAAT |
| | | CAAGACCACTACCCCAGCACCGAGGCCACCCACCCCGGCTCC |
| | | TACCATCGCCTCCCAGCCTCTGTCCCTGCGTCCGGAGGCATGT |
| | | AGACCCGCAGCTGGTGGGGCCGTGCATACCCGGGGTCTTGAC |
| | | TTCGCCTGCGATATCTACATTTGGGCCCCTCTGGCTGGTACTT |
| | | GCGGGGTCCTGCTGCTTTCACTCGTGATCACTCTTTACTGTAA |
| | | GCGCGGTCGGAAGAAGCTGCTGTACATCTTTAAGCAACCCTT |
| | | CATGAGGCCTGTGCAGACTACTCAAGAGGAGGACGGCTGTTC |
| | | ATGCCGGTTCCCAGAGGAGGAGGAAGGCGGCTGCGAACTGC |
| | | GCGTGAAATTCAGCCGCAGCGCAGATGCTCCAGCCTACAAGC |
| | | AGGGGCAGAACCAGCTCTACAACGAACTCAATCTTGGTCGGA |
| | | GAGAGGAGTACGACGTGCTGGACAAGCGGAGAGGACGGGAC |
| | | CCAGAAATGGGCGGGAAGCCGCGCAGAAAGAATCCCCAAGA |
| | | GGGCCTGTACAACGAGCTCCAAAAGGATAAGATGGCAGAAG |
| | | CCTATAGCGAGATTGGTATGAAAGGGGAACGCAGAAGAGGC |
| | | AAAGGCCACGACGGACTGTACCAGGGACTCAGCACCGCCACC |
| | | AAGGACACCTATGACGCTCTTCACATGCAGGCCCTGCCGCCT |
| | | CGG |

139104

| | | |
|---|---|---|
| 139104-aa | 1442 | EVQLLETGGGLVQPGGSLRLSCAVSGFALSNHGMSWVRRAPGK |
| ScFv | | GLEWVSGIVYSGSTYYAASVKGRFTISRDNSRNTLYLQMNSLRP |
| domain | | EDTAIYYCSAHGGESDVWGQGTTVTVSSASGGGGSGGRASGGG |
| | | GSEIVLTQSPATLSVSPGESATLSCRASQSVSSNLAWYQQKPGQA |
| | | PRLLIYGASTRASGIPDRFSGSGSGTDFTLTISSLQAEDVAVYYCQ |
| | | QYGSSLTFGGGTKVEIK |
| 139104-nt | 1443 | GAAGTGCAATTGCTCGAAACTGGAGGAGGTCTGGTGCAACCT |
| ScFv | | GGAGGATCACTTCGCCTGTCCTGCGCCGTGTCGGGCTTTGCCC |
| domain | | TGTCCAACCATGGAATGAGCTGGGTCCGCCGCGCGCCGGGGA |
| | | AGGGCCTCGAATGGGTGTCCGGCATCGTCTACTCCGGCTCCA |
| | | CCTACTACGCCGCGTCCGTGAAGGGCCGGTTCACGATTTCAC |
| | | GGGACAACTCGCGGAACACCCTGTACCTCCAAATGAATTCCC |
| | | TTCGGCCGGAGGATACTGCCATCTACTACTGCTCCGCCCACGG |
| | | TGGCGAATCCGACGTCTGGGGCCAGGGAACCACCGTGACCGT |
| | | GTCCAGCGCGTCCGGGGGAGGAGGAAGCGGGGGTAGAGCAT |
| | | CGGGTGGAGGCGGATCAGAGATCGTGCTGACCCAGTCCCCCG |
| | | CCACCTTGAGCGTGTCACCAGGAGAGTCCGCCACCCTGTCAT |
| | | GCCGCGCCAGCCAGTCCGTGTCCTCCAACCTGGCTTGGTACCA |
| | | GCAGAAGCCGGGGCAGGCCCCTAGACTCCTGATCTATGGGGC |
| | | GTCGACCCGGGCATCTGGAATTCCCGATAGGTTCAGCGGATC |
| | | GGGCTCGGGCACTGACTTCACTCTGACCATCTCCTCGCTGCAA |
| | | GCCGAGGACGTGGCTGTGTACTACTGTCAGCAGTACGGAAGC |
| | | TCCCTGACTTTCGGTGGCGGGACCAAAGTCGAGATTAAG |
| 139104-aa | 1444 | EVQLLETGGGLVQPGGSLRLSCAVSGFALSNHGMSWVRRAPGK |
| VH | | GLEWVSGIVYSGSTYYAASVKGRFTISRDNSRNTLYLQMNSLRP |
| | | EDTAIYYCSAHGGESDVWGQGTTVTVSS |
| 139104-aa | 1445 | EIVLTQSPATLSVSPGESATLSCRASQSVSSNLAWYQQKPGQAPR |
| VL | | LLIYGASTRASGIPDRFSGSGSGTDFTLTISSLQAEDVAVYYCQQ |
| | | YGSSLTFGGGTKVEIK |
| 139104-aa | 1446 | MALPVTALLLPLALLLHAARPEVQLLETGGGLVQPGGSLRLSCA |
| Full CAR | | VSGFALSNHGMSWVRRAPGKGLEWVSGIVYSGSTYYAASVKGR |
| | | FTISRDNSRNTLYLQMNSLRPEDTAIYYCSAHGGESDVWGQGTT |
| | | VTVSSASGGGGSGGRASGGGGSEIVLTQSPATLSVSPGESATLSC |
| | | RASQSVSSNLAWYQQKPGQAPRLLIYGASTRASGIPDRFSGSGSG |
| | | TDFTLTISSLQAEDVAVYYCQQYGSSLTFGGGTKVEIKTTTPAPR |
| | | PPTPAPTIASQPLSLRPEACRPAAGGAVHTRGLDFACDIYIWAPL |
| | | AGTCGVLLLSLVITLYCKRGRKKLLYIFKQPFMRPVQTTQEEDG |
| | | CSCRFPEEEEGGCELRVKFSRSADAPAYKQGQNQLYNELNLGRR |
| | | EEYDVLDKRRGRDPEMGGKPRRKNPQEGLYNELQKDKMAEAY |
| | | SEIGMKGERRRGKGHDGLYQGLSTATKDTYDALHMQALPPR |
| 139104-nt | 1447 | ATGGCCCTCCCTGTCACCGCCCTGCTGCTTCCGCTGGCTCTTC |
| Full CAR | | TGCTCCACGCCGCTCGGCCCGAAGTGCAATTGCTCGAAACTG |
| | | GAGGAGGTCTGGTGCAACCTGGAGGATCACTTCGCCTGTCCT |
| | | GCGCCGTGTCGGGCTTTGCCCTGTCCAACCATGGAATGAGCT |
| | | GGGTCCGCCGCGCGCCGGGGAAGGGCCTCGAATGGGTGTCCG |
| | | GCATCGTCTACTCCGGCTCCACCTACTACGCCGCGTCCGTGAA |
| | | GGGCCGGTTCACGATTTCACGGGACAACTCGCGGAACACCCT |
| | | GTACCTCCAAATGAATTCCCTTCGGCCGGAGGATACTGCCATC |
| | | TACTACTGCTCCGCCCACGGTGGCGAATCCGACGTCTGGGGC |
| | | CAGGGAACCACCGTGACCGTGTCCAGCGCGTCCGGGGGAGGA |
| | | GGAAGCGGGGGTAGAGCATCGGGTGGAGGCGGATCAGAGAT |
| | | CGTGCTGACCCAGTCCCCCGCCACCTTGAGCGTGTCACCAGG |
| | | AGAGTCCGCCACCCTGTCATGCCGCGCCAGCCAGTCCGTGTC |
| | | CTCCAACCTGGCTTGGTACCAGCAGAAGCCGGGGCAGGCCCC |
| | | TAGACTCCTGATCTATGGGGCGTCGACCCGGGCATCTGGAAT |
| | | TCCCGATAGGTTCAGCGGATCGGGCTCGGGCACTGACTTCAC |
| | | TCTGACCATCTCCTCGCTGCAAGCCGAGGACGTGGCTGTGTAC |
| | | TACTGTCAGCAGTACGGAAGCTCCCTGACTTTCGGTGGCGGG |
| | | ACCAAAGTCGAGATTAAGACCACTACCCCAGCACCGAGGCCA |
| | | CCCACCCCGGCTCCTACCATCGCCTCCCAGCCTCTGTCCCTGC |
| | | GTCCGGAGGCATGTAGACCCGCAGCTGGTGGGGCCGTGCATA |
| | | CCCGGGGTCTTGACTTCGCCTGCGATATCTACATTTGGGCCCC |
| | | TCTGGCTGGTACTTGCGGGGTCCTGCTGCTTTCACTCGTGATC |
| | | ACTCTTTACTGTAAGCGCGGTCGGAAGAAGCTGCTGTACATCT |
| | | TTAAGCAACCCTTCATGAGGCCTGTGCAGACTACTCAAGAGG |
| | | AGGACGGCTGTTCATGCCGGTTCCCAGAGGAGGAGGAAGGCG |
| | | GCTGCGAACTGCGCGTGAAATTCAGCCGCAGCGCAGATGCTC |
| | | CAGCCTACAAGCAGGGGCAGAACCAGCTCTACAACGAACTCA |
| | | ATCTTGGTCGGAGAGAGGAGTACGACGTGCTGGACAAGCGGA |
| | | GAGGACGGGACCCAGAAATGGGCGGGAAGCCGCGCAGAAAG |
| | | AATCCCCAAGAGGGCCTGTACAACGAGCTCCAAAAGGATAAG |
| | | ATGGCAGAAGCCTATAGCGAGATTGGTATGAAAGGGGAACG |
| | | CAGAAGAGGCAAAGGCCACGACGGACTGTACCAGGGACTCA |
| | | GCACCGCCACCAAGGACACCTATGACGCTCTTCACATGCAGG |
| | | CCCTGCCGCCTCGG |

139106

| | | |
|---|---|---|
| 139106-aa | 1448 | EVQLVETGGGLVQPGGSLRLSCAVSGFALSNHGMSWVRRAPGK |
| ScFv | | GLEWVSGIVYSGSTYYAASVKGRFTISRDNSRNTLYLQMNSLRP |
| domain | | EDTAIYYCSAHGGESDVWGQGTTVTVSSASGGGGSGGRASGGG |
| | | GSEIVMTQSPATLSVSPGERATLSCRASQSVSSKLAWYQQKPGQ |
| | | APRLLMYGASIRATGIPDRFSGSGSGTEFTLTISSLEPEDFAVYYC |
| | | QQYGSSSWTFGQGTKVEIK |
| 139106-nt | 1449 | GAAGTGCAATTGGTGGAAACTGGAGGAGGACTTGTGCAACCT |
| ScFv | | GGAGGATCATTGAGACTGAGCTGCGCAGTGTCGGGATTCGCC |
| domain | | CTGAGCAACCATGGAATGTCCTGGGTCAGAAGGGCCCCTGGA |
| | | AAAGGCCTCGAATGGGTGTCAGGGATCGTGTACTCCGGTTCC |
| | | ACTTACTACGCCGCCTCCGTGAAGGGGCGCTTCACTATCTCAC |
| | | GGGATAACTCCCGCAATACCCTGTACCTCCAAATGAACAGCC |
| | | TGCGGCCGGAGGATACCGCCATCTACTACTGTTCCGCCCACG |
| | | GTGGAGAGTCTGACGTCTGGGGCCAGGGAACTACCGTGACCG |
| | | TGTCCTCCGCGTCCGGCGGTGGAGGGAGCGGCGGCCGCGCCA |
| | | GCGGCGGCGGAGGCTCCGAGATCGTGATGACCCAGAGCCCCG |
| | | CTACTCTGTCGGTGTCGCCCGGAGAAAGGGCGACCCTGTCCT |
| | | GCCGGGCGTCGCAGTCCGTGAGCAGCAAGCTGGCTTGGTACC |
| | | AGCAGAAGCCGGGCCAGGCACCACGCCTGCTTATGTACGGTG |
| | | CCTCCATTCGGGCCACCGGAATCCCGGACCGGTTCTCGGGGT |
| | | CGGGGTCCGGTACCGAGTTCACACTGACCATTTCCTCGCTCGA |
| | | GCCCGAGGACTTTGCCGTCTATTACTGCCAGCAGTACGGCTCC |
| | | TCCTCATGGACGTTCGGCCAGGGGACCAAGGTCGAAATCAAG |
| 139106-aa | 1450 | EVQLVETGGGLVQPGGSLRLSCAVSGFALSNHGMSWVRRAPGK |
| VH | | GLEWVSGIVYSGSTYYAASVKGRFTISRDNSRNTLYLQMNSLRP |
| | | EDTAIYYCSAHGGESDVWGQGTTVTVSS |
| 139106-aa | 1451 | EIVMTQSPATLSVSPGERATLSCRASQSVSSKLAWYQQKPGQAP |
| VL | | RLLMYGASIRATGIPDRFSGSGSGTEFTLTISSLEPEDFAVYYCQQ |
| | | YGSSSWTFGQGTKVEIK |
| 139106-aa | 1452 | MALPVTALLLPLALLLHAARPEVQLVETGGGLVQPGGSLRLSCA |
| Full CAR | | VSGFALSNHGMSWVRRAPGKGLEWVSGIVYSGSTYYAASVKGR |
| | | FTISRDNSRNTLYLQMNSLRPEDTAIYYCSAHGGESDVWGQGTT |
| | | VTVSSASGGGGSGGRASGGGGSEIVMTQSPATLSVSPGERATLS |
| | | CRASQSVSSKLAWYQQKPGQAPRLLMYGASIRATGIPDRFSGSG |
| | | SGTEFTLTISSLEPEDFAVYYCQQYGSSSWTFGQGTKVEIKTTTP |
| | | APRPPTPAPTIASQPLSLRPEACRPAAGGAVHTRGLDFACDIYIW |
| | | APLAGTCGVLLLSLVITLYCKRGRKKLLYIFKQPFMRPVQTTQEE |
| | | DGCSCRFPEEEEGGCELRVKFSRSADAPAYKQGQNQLYNELNLG |
| | | RREEYDVLDKRRGRDPEMGGKPRRKNPQEGLYNELQKDKMAE |
| | | AYSEIGMKGERRRGKGHDGLYQGLSTATKDTYDALHMQALPPR |
| 139106-nt | 1453 | ATGGCCCTCCCTGTCACCGCCCTGCTGCTTCCGCTGGCTCTTC |
| Full CAR | | TGCTCCACGCCGCTCGGCCCGAAGTGCAATTGGTGGAAACTG |
| | | GAGGAGGACTTGTGCAACCTGGAGGATCATTGAGACTGAGCT |
| | | GCGCAGTGTCGGGATTCGCCCTGAGCAACCATGGAATGTCCT |
| | | GGGTCAGAAGGGCCCCTGGAAAAGGCCTCGAATGGGTGTCAG |
| | | GGATCGTGTACTCCGGTTCCACTTACTACGCCGCCTCCGTGAA |
| | | GGGGCGCTTCACTATCTCACGGGATAACTCCCGCAATACCCT |
| | | GTACCTCCAAATGAACAGCCTGCGGCCGGAGGATACCGCCAT |
| | | CTACTACTGTTCCGCCCACGGTGGAGAGTCTGACGTCTGGGG |
| | | CCAGGGAACTACCGTGACCGTGTCCTCCGCGTCCGGCGGTGG |
| | | AGGGAGCGGCGGCCGCGCCAGCGGCGGCGGAGGCTCCGAGA |
| | | TCGTGATGACCCAGAGCCCCGCTACTCTGTCGGTGTCGCCCGG |
| | | AGAAAGGGCGACCCTGTCCTGCCGGGCGTCGCAGTCCGTGAG |
| | | CAGCAAGCTGGCTTGGTACCAGCAGAAGCCGGGCCAGGCACC |
| | | ACGCCTGCTTATGTACGGTGCCTCCATTCGGGCCACCGGAATC |
| | | CCGGACCGGTTCTCGGGGTCGGGGTCCGGTACCGAGTTCACA |
| | | CTGACCATTTCCTCGCTCGAGCCCGAGGACTTTGCCGTCTATT |
| | | ACTGCCAGCAGTACGGCTCCTCCTCATGGACGTTCGGCCAGG |
| | | GGACCAAGGTCGAAATCAAGACCACTACCCCAGCACCGAGGC |
| | | CACCCACCCCGGCTCCTACCATCGCCTCCCAGCCTCTGTCCCT |
| | | GCGTCCGGAGGCATGTAGACCCGCAGCTGGTGGGGCCGTGCA |
| | | TACCCGGGGTCTTGACTTCGCCTGCGATATCTACATTTGGGCC |
| | | CCTCTGGCTGGTACTTGCGGGGTCCTGCTGCTTTCACTCGTGA |
| | | TCACTCTTTACTGTAAGCGCGGTCGGAAGAAGCTGCTGTACAT |
| | | CTTTAAGCAACCCTTCATGAGGCCTGTGCAGACTACTCAAGA |
| | | GGAGGACGGCTGTTCATGCCGGTTCCCAGAGGAGGAGGAAG |
| | | GCGGCTGCGAACTGCGCGTGAAATTCAGCCGCAGCGCAGATG |
| | | CTCCAGCCTACAAGCAGGGGCAGAACCAGCTCTACAACGAAC |
| | | TCAATCTTGGTCGGAGAGAGGAGTACGACGTGCTGGACAAGC |
| | | GGAGAGGACGGGACCCAGAAATGGGCGGGAAGCCGCGCAGA |
| | | AAGAATCCCCAAGAGGGCCTGTACAACGAGCTCCAAAAGGAT |
| | | AAGATGGCAGAAGCCTATAGCGAGATTGGTATGAAAGGGGA |
| | | ACGCAGAAGAGGCAAAGGCCACGACGGACTGTACCAGGGAC |
| | | TCAGCACCGCCACCAAGGACACCTATGACGCTCTTCACATGC |
| | | AGGCCCTGCCGCCTCGG |

139107

| | | |
|---|---|---|
| 139107-aa | 1454 | EVQLVETGGGVVQPGGSLRLSCAVSGFALSNHGMSWVRRAPGK |
| ScFv | | GLEWVSGIVYSGSTYYAASVKGRFTISRDNSRNTLYLQMNSLRP |
| domain | | EDTAIYYCSAHGGESDVWGQGTTVTVSSASGGGGSGGRASGGG |
| | | GSEIVLTQSPGTLSLSPGERATLSCRASQSVGSTNLAWYQQKPGQ |
| | | APRLLIYDASNRATGIPDRFSGGGSGTDFTLTISRLEPEDFAVYYC |
| | | QQYGSSPPWTFGQGTKVEIK |
| 139107-nt | 1455 | GAAGTGCAATTGGTGGAGACTGGAGGAGGAGTGGTGCAACCT |
| ScFv | | GGAGGAAGCCTGAGACTGTCATGCGCGGTGTCGGGCTTCGCC |
| domain | | CTCTCCAACCACGGAATGTCCTGGGTCCGCCGGGCCCCTGGG |
| | | AAAGGACTTGAATGGGTGTCCGGCATCGTGTACTCGGGTTCC |
| | | ACCTACTACGCGGCCTCAGTGAAGGGCCGGTTTACTATTAGC |
| | | CGCGACAACTCCAGAAACACACTGTACCTCCAAATGAACTCG |
| | | CTGCGGCCGGAAGATACCGCTATCTACTACTGCTCCGCCCATG |
| | | GGGGAGAGTCGGACGTCTGGGGACAGGGCACCACTGTCACTG |
| | | TGTCCAGCGCTTCCGGCGGTGGTGGAAGCGGGGGACGGGCCT |
| | | CAGGAGGCGGTGGCAGCGAGATTGTGCTGACCCAGTCCCCCG |
| | | GGACCCTGAGCCTGTCCCCGGGAGAAAGGGCCACCCTCTCCT |
| | | GTCGGGCATCCCAGTCCGTGGGGTCTACTAACCTTGCATGGTA |
| | | CCAGCAGAAGCCCGGCCAGGCCCCTCGCCTGCTGATCTACGA |
| | | CGCGTCCAATAGAGCCACCGGCATCCCGGATCGCTTCAGCGG |
| | | AGGCGGATCGGGCACCGACTTCACCCTCACCATTTCAAGGCT |
| | | GGAACCGGAGGACTTCGCCGTGTACTACTGCCAGCAGTATGG |
| | | TTCGTCCCCACCCTGGACGTTCGGCCAGGGGACTAAGGTCGA |
| | | GATCAAG |
| 139107-aa | 1456 | EVQLVETGGGVVQPGGSLRLSCAVSGFALSNHGMSWVRRAPGK |
| VH | | GLEWVSGIVYSGSTYYAASVKGRFTISRDNSRNTLYLQMNSLRP |
| | | EDTAIYYCSAHGGESDVWGQGTTVTVSS |
| 139107-aa | 1457 | EIVLTQSPGTLSLSPGERATLSCRASQSVGSTNLAWYQQKPGQAP |
| VL | | RLLIYDASNRATGIPDRFSGGGSGTDFTLTISRLEPEDFAVYYCQQ |
| | | YGSSPPWTFGQGTKVEIK |
| 139107-aa | 1458 | MALPVTALLLPLALLLHAARPEVQLVETGGGVVQPGGSLRLSCA |
| Full CAR | | VSGFALSNHGMSWVRRAPGKGLEWVSGIVYSGSTYYAASVKGR |
| | | FTISRDNSRNTLYLQMNSLRPEDTAIYYCSAHGGESDVWGQGTT |
| | | VTVSSASGGGGSGGRASGGGGSEIVLTQSPGTLSLSPGERATLSC |
| | | RASQSVGSTNLAWYQQKPGQAPRLLIYDASNRATGIPDRFSGGG |
| | | SGTDFTLTISRLEPEDFAVYYCQQYGSSPPWTFGQGTKVEIKTTT |
| | | PAPRPPTPAPTIASQPLSLRPEACRPAAGGAVHTRGLDFACDIYIW |
| | | APLAGTCGVLLLSLVITLYCKRGRKKLLYIFKQPFMRPVQTTQEE |
| | | DGCSCRFPEEEEGGCELRVKFSRSADAPAYKQGQNQLYNELNLG |
| | | RREEYDVLDKRRGRDPEMGGKPRRKNPQEGLYNELQKDKMAE |
| | | AYSEIGMKGERRRGKGHDGLYQGLSTATKDTYDALHMQALPPR |
| 139107-nt | 1459 | ATGGCCCTCCCTGTCACCGCCCTGCTGCTTCCGCTGGCTCTTC |
| Full CAR | | TGCTCCACGCCGCTCGGCCCGAAGTGCAATTGGTGGAGACTG |
| | | GAGGAGGAGTGGTGCAACCTGGAGGAAGCCTGAGACTGTCAT |
| | | GCGCGGTGTCGGGCTTCGCCCTCTCCAACCACGGAATGTCCTG |
| | | GGTCCGCCGGGCCCCTGGGAAAGGACTTGAATGGGTGTCCGG |
| | | CATCGTGTACTCGGGTTCCACCTACTACGCGGCCTCAGTGAAG |
| | | GGCCGGTTTACTATTAGCCGCGACAACTCCAGAAACACACTG |
| | | TACCTCCAAATGAACTCGCTGCGGCCGGAAGATACCGCTATC |
| | | TACTACTGCTCCGCCCATGGGGGAGAGTCGGACGTCTGGGGA |
| | | CAGGGCACCACTGTCACTGTGTCCAGCGCTTCCGGCGGTGGT |
| | | GGAAGCGGGGGACGGGCCTCAGGAGGCGGTGGCAGCGAGAT |
| | | TGTGCTGACCCAGTCCCCCGGGACCCTGAGCCTGTCCCCGGG |
| | | AGAAAGGGCCACCCTCTCCTGTCGGGCATCCCAGTCCGTGGG |
| | | GTCTACTAACCTTGCATGGTACCAGCAGAAGCCCGGCCAGGC |
| | | CCCTCGCCTGCTGATCTACGACGCGTCCAATAGAGCCACCGG |
| | | CATCCCGGATCGCTTCAGCGGAGGCGGATCGGGCACCGACTT |
| | | CACCCTCACCATTTCAAGGCTGGAACCGGAGGACTTCGCCGT |
| | | GTACTACTGCCAGCAGTATGGTTCGTCCCCACCCTGGACGTTC |
| | | GGCCAGGGGACTAAGGTCGAGATCAAGACCACTACCCCAGCA |
| | | CCGAGGCCACCCACCCCGGCTCCTACCATCGCCTCCCAGCCTC |
| | | TGTCCCTGCGTCCGGAGGCATGTAGACCCGCAGCTGGTGGGG |
| | | CCGTGCATACCCGGGGTCTTGACTTCGCCTGCGATATCTACAT |
| | | TTGGGCCCCTCTGGCTGGTACTTGCGGGGTCCTGCTGCTTTCA |
| | | CTCGTGATCACTCTTTACTGTAAGCGCGGTCGGAAGAAGCTG |
| | | CTGTACATCTTTAAGCAACCCTTCATGAGGCCTGTGCAGACTA |
| | | CTCAAGAGGAGGACGGCTGTTCATGCCGGTTCCCAGAGGAGG |
| | | AGGAAGGCGGCTGCGAACTGCGCGTGAAATTCAGCCGCAGCG |
| | | CAGATGCTCCAGCCTACAAGCAGGGGCAGAACCAGCTCTACA |
| | | ACGAACTCAATCTTGGTCGGAGAGAGGAGTACGACGTGCTGG |
| | | ACAAGCGGAGAGGACGGGACCCAGAAATGGGCGGGAAGCCG |
| | | CGCAGAAAGAATCCCCAAGAGGGCCTGTACAACGAGCTCCAA |
| | | AAGGATAAGATGGCAGAAGCCTATAGCGAGATTGGTATGAA |
| | | AGGGGAACGCAGAAGAGGCAAAGGCCACGACGGACTGTACC |
| | | AGGGACTCAGCACCGCCACCAAGGACACCTATGACGCTCTTC |
| | | ACATGCAGGCCCTGCCGCCTCGG |

139108

| | | |
|---|---|---|
| 139108-aa | 1460 | QVQLVESGGGLVKPGGSLRLSCAASGFTFSDYYMSWIRQAPGK |
| ScFv | | GLEWVSYISSSGSTIYYADSVKGRFTISRDNAKNSLYLQMNSLRA |
| domain | | EDTAVYYCARESGDGMDVWGQGTTVTVSSASGGGGSGGRASG |
| | | GGGSDIQMTQSPSSLSASVGDRVTITCRASQSISSYLNWYQQKPG |
| | | KAPKLLIYAASSLQSGVPSRFSGSGSGTDFTLTISSLQPEDFATYY |
| | | CQQSYTLAFGQGTKVDIK |
| 139108-nt | 1461 | CAAGTGCAACTCGTGGAATCTGGTGGAGGACTCGTGAAACCT |
| ScFv | | GGAGGATCATTGAGACTGTCATGCGCGGCCTCGGGATTCACG |
| domain | | TTCTCCGATTACTACATGAGCTGGATTCGCCAGGCTCCGGGGA |
| | | AGGGACTGGAATGGGTGTCCTACATTTCCTCATCCGGCTCCAC |
| | | CATCTACTACGCGGACTCCGTGAAGGGGAGATTCACCATTAG |
| | | CCGCGATAACGCCAAGAACAGCCTGTACCTTCAGATGAACTC |
| | | CCTGCGGGCTGAAGATACTGCCGTCTACTACTGCGCAAGGGA |
| | | GAGCGGAGATGGGATGGACGTCTGGGGACAGGGTACCACTGT |
| | | GACCGTGTCGTCGGCCTCCGGCGGAGGGGGTTCGGGTGGAAG |
| | | GGCCAGCGGCGGCGGAGGCAGCGACATCCAGATGACCCAGT |
| | | CCCCCTCATCGCTGTCCGCCTCCGTGGGCGACCGCGTCACCAT |
| | | CACATGCCGGGCCTCACAGTCGATCTCCTCCTACCTCAATTGG |
| | | TATCAGCAGAAGCCCGGAAAGGCCCCTAAGCTTCTGATCTAC |
| | | GCAGCGTCCTCCCTGCAATCCGGGGTCCCATCTCGGTTCTCCG |
| | | GCTCGGGCAGCGGTACCGACTTCACTCTGACCATCTCGAGCCT |
| | | GCAGCCGGAGGACTTCGCCACTTACTACTGTCAGCAAAGCTA |
| | | CACCCTCGCGTTTGGCCAGGGCACCAAAGTGGACATCAAG |
| 139108-aa | 1462 | QVQLVESGGGLVKPGGSLRLSCAASGFTFSDYYMSWIRQAPGK |
| VH | | GLEWVSYISSSGSTIYYADSVKGRFTISRDNAKNSLYLQMNSLRA |
| | | EDTAVYYCARESGDGMDVWGQGTTVTVSS |
| 139108-aa | 1463 | DIQMTQSPSSLSASVGDRVTITCRASQSISSYLNWYQQKPGKAPK |
| VL | | LLIYAASSLQSGVPSRFSGSGSGTDFTLTISSLQPEDFATYYCQQS |
| | | YTLAFGQGTKVDIK |
| 139108-aa | 1464 | MALPVTALLLPLALLLHAARPQVQLVESGGGLVKPGGSLRLSCA |
| Full CAR | | ASGFTFSDYYMSWIRQAPGKGLEWVSYISSSGSTIYYADSVKGR |
| | | FTISRDNAKNSLYLQMNSLRAEDTAVYYCARESGDGMDVWGQ |
| | | GTTVTVSSASGGGGSGGRASGGGGSDIQMTQSPSSLSASVGDRV |
| | | TITCRASQSISSYLNWYQQKPGKAPKLLIYAASSLQSGVPSRFSGS |
| | | GSGTDFTLTISSLQPEDFATYYCQQSYTLAFGQGTKVDIKTTTPA |
| | | PRPPTPAPTIASQPLSLRPEACRPAAGGAVHTRGLDFACDIYIWAP |
| | | LAGTCGVLLLSLVITLYCKRGRKKLLYIFKQPFMRPVQTTQEED |
| | | GCSCRFPEEEEGGCELRVKFSRSADAPAYKQGQNQLYNELNLGR |
| | | REEYDVLDKRRGRDPEMGGKPRRKNPQEGLYNELQKDKMAEA |
| | | YSEIGMKGERRRGKGHDGLYQGLSTATKDTYDALHMQALPPR |
| 139108-nt | 1465 | ATGGCCCTCCCTGTCACCGCCCTGCTGCTTCCGCTGGCTCTTC |
| Full CAR | | TGCTCCACGCCGCTCGGCCCCAAGTGCAACTCGTGGAATCTG |
| | | GTGGAGGACTCGTGAAACCTGGAGGATCATTGAGACTGTCAT |
| | | GCGCGGCCTCGGGATTCACGTTCTCCGATTACTACATGAGCTG |
| | | GATTCGCCAGGCTCCGGGGAAGGGACTGGAATGGGTGTCCTA |
| | | CATTTCCTCATCCGGCTCCACCATCTACTACGCGGACTCCGTG |
| | | AAGGGGAGATTCACCATTAGCCGCGATAACGCCAAGAACAGC |
| | | CTGTACCTTCAGATGAACTCCCTGCGGGCTGAAGATACTGCC |
| | | GTCTACTACTGCGCAAGGGAGAGCGGAGATGGGATGGACGTC |
| | | TGGGGACAGGGTACCACTGTGACCGTGTCGTCGGCCTCCGGC |
| | | GGAGGGGGTTCGGGTGGAAGGGCCAGCGGCGGCGGAGGCAG |
| | | CGACATCCAGATGACCCAGTCCCCCTCATCGCTGTCCGCCTCC |
| | | GTGGGCGACCGCGTCACCATCACATGCCGGGCCTCACAGTCG |
| | | ATCTCCTCCTACCTCAATTGGTATCAGCAGAAGCCCGGAAAG |
| | | GCCCCTAAGCTTCTGATCTACGCAGCGTCCTCCCTGCAATCCG |
| | | GGGTCCCATCTCGGTTCTCCGGCTCGGGCAGCGGTACCGACTT |
| | | CACTCTGACCATCTCGAGCCTGCAGCCGGAGGACTTCGCCAC |
| | | TTACTACTGTCAGCAAAGCTACACCCTCGCGTTTGGCCAGGGC |
| | | ACCAAAGTGGACATCAAGACCACTACCCCAGCACCGAGGCCA |
| | | CCCACCCCGGCTCCTACCATCGCCTCCCAGCCTCTGTCCCTGC |
| | | GTCCGGAGGCATGTAGACCCGCAGCTGGTGGGGCCGTGCATA |
| | | CCCGGGGTCTTGACTTCGCCTGCGATATCTACATTTGGGCCCC |
| | | TCTGGCTGGTACTTGCGGGGTCCTGCTGCTTTCACTCGTGATC |
| | | ACTCTTTACTGTAAGCGCGGTCGGAAGAAGCTGCTGTACATCT |
| | | TTAAGCAACCCTTCATGAGGCCTGTGCAGACTACTCAAGAGG |
| | | AGGACGGCTGTTCATGCCGGTTCCCAGAGGAGGAGGAAGGCG |
| | | GCTGCGAACTGCGCGTGAAATTCAGCCGCAGCGCAGATGCTC |
| | | CAGCCTACAAGCAGGGGCAGAACCAGCTCTACAACGAACTCA |
| | | ATCTTGGTCGGAGAGAGGAGTACGACGTGCTGGACAAGCGGA |
| | | GAGGACGGGACCCAGAAATGGGCGGGAAGCCGCGCAGAAAG |
| | | AATCCCCAAGAGGGCCTGTACAACGAGCTCCAAAAGGATAAG |
| | | ATGGCAGAAGCCTATAGCGAGATTGGTATGAAAGGGGAACG |
| | | CAGAAGAGGCAAAGGCCACGACGGACTGTACCAGGGACTCA |
| | | GCACCGCCACCAAGGACACCTATGACGCTCTTCACATGCAGG |
| | | CCCTGCCGCCTCGG |

139110

| | | |
|---|---|---|
| 139110-aa | 1466 | QVQLVQSGGGLVKPGGSLRLSCAASGFTFSDYYMSWIRQAPGK |
| ScFv | | GLEWVSYISSSGNTIYYADSVKGRFTISRDNAKNSLYLQMNSLR |
| domain | | AEDTAVYYCARSTMVREDYWGQGTLVTVSSASGGGGSGGRAS |
| | | GGGGSDIVLTQSPLSLPVTLGQPASISCKSSESLVHNSGKTYLNW |
| | | FHQRPGQSPRRLIYEVSNRDSGVPDRFTGSGSGTDFTLKISRVEA |
| | | EDVGVYYCMQGTHWPGTFGQGTKLEIK |
| 139110-nt | 1467 | CAAGTGCAACTGGTGCAAAGCGGAGGAGGATTGGTCAAACCC |
| ScFv | | GGAGGAAGCCTGAGACTGTCATGCGCGGCCTCTGGATTCACC |
| domain | | TTCTCCGATTACTACATGTCATGGATCAGACAGGCCCCGGGG |
| | | AAGGGCCTCGAATGGGTGTCCTACATCTCGTCCTCCGGGAAC |
| | | ACCATCTACTACGCCGACAGCGTGAAGGGCCGCTTTACCATTT |
| | | CCCGCGACAACGCAAAGAACTCGCTGTACCTTCAGATGAATT |
| | | CCCTGCGGGCTGAAGATACCGCGGTGTACTATTGCGCCCGGT |
| | | CCACTATGGTCCGGGAGGACTACTGGGGACAGGGCACACTCG |
| | | TGACCGTGTCCAGCGCGAGCGGGGGTGGAGGCAGCGGTGGA |
| | | CGCGCCTCCGGCGGCGGCGGTTCAGACATCGTGCTGACTCAG |
| | | TCGCCCCTGTCGCTGCCGGTCACCCTGGGCCAACCGGCCTCAA |
| | | TTAGCTGCAAGTCCTCGGAGAGCCTGGTGCACAACTCAGGAA |
| | | AGACTTACCTGAACTGGTTCCATCAGCGGCCTGGACAGTCCC |
| | | CACGGAGGCTCATCTATGAAGTGTCCAACAGGGATTCGGGGG |
| | | TGCCCGACCGCTTCACTGGCTCCGGGTCCGGCACCGACTTCAC |
| | | CTTGAAAATCTCCAGAGTGGAAGCCGAGGACGTGGGCGTGTA |
| | | CTACTGTATGCAGGGTACCCACTGGCCTGGAACCTTTGGACA |
| | | AGGAACTAAGCTCGAGATTAAG |
| 139110-aa | 1468 | QVQLVQSGGGLVKPGGSLRLSCAASGFTFSDYYMSWIRQAPGK |
| VH | | GLEWVSYISSSGNTIYYADSVKGRFTISRDNAKNSLYLQMNSLR |
| | | AEDTAVYYCARSTMVREDYWGQGTLVTVSS |
| 139110-aa | 1469 | DIVLTQSPLSLPVTLGQPASISCKSSESLVHNSGKTYLNWFHQRP |
| VL | | GQSPRRLIYEVSNRDSGVPDRFTGSGSGTDFTLKISRVEAEDVGV |
| | | YYCMQGTHWPGTFGQGTKLEIK |
| 139110-aa | 1470 | MALPVTALLLPLALLLHAARPQVQLVQSGGGLVKPGGSLRLSCA |
| Full CAR | | ASGFTFSDYYMSWIRQAPGKGLEWVSYISSSGNTIYYADSVKGR |
| | | FTISRDNAKNSLYLQMNSLRAEDTAVYYCARSTMVREDYWGQ |
| | | GTLVTVSSASGGGGSGGRASGGGGSDIVLTQSPLSLPVTLGQPAS |
| | | ISCKSSESLVHNSGKTYLNWFHQRPGQSPRRLIYEVSNRDSGVPD |
| | | RFTGSGSGTDFTLKISRVEAEDVGVYYCMQGTHWPGTFGQGTK |
| | | LEIKTTTPAPRPPTPAPTIASQPLSLRPEACRPAAGGAVHTRGLDF |
| | | ACDIYIWAPLAGTCGVLLLSLVITLYCKRGRKKLLYIFKQPFMRP |
| | | VQTTQEEDGCSCRFPEEEEGGCELRVKFSRSADAPAYKQGQNQL |
| | | YNELNLGRREEYDVLDKRRGRDPEMGGKPRRKNPQEGLYNELQ |
| | | KDKMAEAYSEIGMKGERRRGKGHDGLYQGLSTATKDTYDALH |
| | | MQALPPR |
| 139110-nt | 1471 | ATGGCCCTCCCTGTCACCGCCCTGCTGCTTCCGCTGGCTCTTC |
| Full CAR | | TGCTCCACGCCGCTCGGCCCCAAGTGCAACTGGTGCAAAGCG |
| | | GAGGAGGATTGGTCAAACCCGGAGGAAGCCTGAGACTGTCAT |
| | | GCGCGGCCTCTGGATTCACCTTCTCCGATTACTACATGTCATG |
| | | GATCAGACAGGCCCCGGGGAAGGGCCTCGAATGGGTGTCCTA |
| | | CATCTCGTCCTCCGGGAACACCATCTACTACGCCGACAGCGT |
| | | GAAGGGCCGCTTTACCATTTCCCGCGACAACGCAAAGAACTC |
| | | GCTGTACCTTCAGATGAATTCCCTGCGGGCTGAAGATACCGC |
| | | GGTGTACTATTGCGCCCGGTCCACTATGGTCCGGGAGGACTA |
| | | CTGGGGACAGGGCACACTCGTGACCGTGTCCAGCGCGAGCGG |
| | | GGGTGGAGGCAGCGGTGGACGCGCCTCCGGCGGCGGCGGTTC |
| | | AGACATCGTGCTGACTCAGTCGCCCCTGTCGCTGCCGGTCACC |
| | | CTGGGCCAACCGGCCTCAATTAGCTGCAAGTCCTCGGAGAGC |
| | | CTGGTGCACAACTCAGGAAAGACTTACCTGAACTGGTTCCAT |
| | | CAGCGGCCTGGACAGTCCCCACGGAGGCTCATCTATGAAGTG |
| | | TCCAACAGGGATTCGGGGGTGCCCGACCGCTTCACTGGCTCC |
| | | GGGTCCGGCACCGACTTCACCTTGAAAATCTCCAGAGTGGAA |
| | | GCCGAGGACGTGGGCGTGTACTACTGTATGCAGGGTACCCAC |
| | | TGGCCTGGAACCTTTGGACAAGGAACTAAGCTCGAGATTAAG |
| | | ACCACTACCCCAGCACCGAGGCCACCCACCCCGGCTCCTACC |
| | | ATCGCCTCCCAGCCTCTGTCCCTGCGTCCGGAGGCATGTAGAC |
| | | CCGCAGCTGGTGGGGCCGTGCATACCCGGGGTCTTGACTTCG |
| | | CCTGCGATATCTACATTTGGGCCCCTCTGGCTGGTACTTGCGG |
| | | GGTCCTGCTGCTTTCACTCGTGATCACTCTTTACTGTAAGCGC |
| | | GGTCGGAAGAAGCTGCTGTACATCTTTAAGCAACCCTTCATG |
| | | AGGCCTGTGCAGACTACTCAAGAGGAGGACGGCTGTTCATGC |
| | | CGGTTCCCAGAGGAGGAGGAAGGCGGCTGCGAACTGCGCGT |
| | | GAAATTCAGCCGCAGCGCAGATGCTCCAGCCTACAAGCAGGG |
| | | GCAGAACCAGCTCTACAACGAACTCAATCTTGGTCGGAGAGA |
| | | GGAGTACGACGTGCTGGACAAGCGGAGAGGACGGGACCCAG |
| | | AAATGGGCGGGAAGCCGCGCAGAAAGAATCCCCAAGAGGGC |
| | | CTGTACAACGAGCTCCAAAAGGATAAGATGGCAGAAGCCTAT |
| | | AGCGAGATTGGTATGAAAGGGGAACGCAGAAGAGGCAAAGG |
| | | CCACGACGGACTGTACCAGGGACTCAGCACCGCCACCAAGGA |
| | | CACCTATGACGCTCTTCACATGCAGGCCCTGCCGCCTCGG |

139112

| | | |
|---|---|---|
| 139112-aa | 1472 | QVQLVESGGGLVQPGGSLRLSCAVSGFALSNHGMSWVRRAPGK |
| ScFv | | GLEWVSGIVYSGSTYYAASVKGRFTISRDNSRNTLYLQMNSLRP |
| domain | | EDTAIYYCSAHGGESDVWGQGTTVTVSSASGGGGSGGRASGGG |
| | | GSDIRLTQSPSPLSASVGDRVTITCQASEDINKFLNWYHQTPGKA |
| | | PKLLIYDASTLQTGVPSRFSGSGSGTDFTLTINSLQPEDIGTYYCQ |
| | | QYESLPLTFGGGTKVEIK |
| 139112-nt | 1473 | CAAGTGCAACTCGTGGAATCTGGTGGAGGACTCGTGCAACCC |
| ScFv | | GGTGGAAGCCTTAGGCTGTCGTGCGCCGTCAGCGGGTTTGCT |
| domain | | CTGAGCAACCATGGAATGTCCTGGGTCCGCCGGGCACCGGGA |
| | | AAAGGGCTGGAATGGGTGTCCGGCATCGTGTACAGCGGGTCA |
| | | ACCTATTACGCCGCGTCCGTGAAGGGCAGATTCACTATCTCA |
| | | AGAGACAACAGCCGGAACACCCTGTACTTGCAAATGAATTCC |
| | | CTGCGCCCCGAGGACACCGCCATCTACTACTGCTCCGCCCAC |
| | | GGAGGAGAGTCGGACGTGTGGGGCCAGGGAACGACTGTGAC |
| | | TGTGTCCAGCGCATCAGGAGGGGGTGGTTCGGGCGGCCGGGC |
| | | CTCGGGGGGAGGAGGTTCCGACATTCGGCTGACCCAGTCCCC |
| | | GTCCCCACTGTCGGCCTCCGTCGGCGACCGCGTGACCATCACT |
| | | TGTCAGGCGTCCGAGGACATTAACAAGTTCCTGAACTGGTAC |
| | | CACCAGACCCCTGGAAAGGCCCCCAAGCTGCTGATCTACGAT |
| | | GCCTCGACCCTTCAAACTGGAGTGCCTAGCCGGTTCTCCGGGT |
| | | CCGGCTCCGGCACTGATTTCACTCTGACCATCAACTCATTGCA |
| | | GCCGGAAGATATCGGGACCTACTATTGCCAGCAGTACGAATC |
| | | CCTCCCGCTCACATTCGGCGGGGGAACCAAGGTCGAGATTAAG |
| 139112-aa | 1474 | QVQLVESGGGLVQPGGSLRLSCAVSGFALSNHGMSWVRRAPGK |
| VH | | GLEWVSGIVYSGSTYYAASVKGRFTISRDNSRNTLYLQMNSLRP |
| | | EDTAIYYCSAHGGESDVWGQGTTVTVSS |
| 139112-aa | 1475 | DIRLTQSPSPLSASVGDRVTITCQASEDINKFLNWYHQTPGKAPK |
| VL | | LLIYDASTLQTGVPSRFSGSGSGTDFTLTINSLQPEDIGTYYCQQY |
| | | ESLPLTFGGGTKVEIK |
| 139112-aa | 1476 | MALPVTALLLPLALLLHAARPQVQLVESGGGLVQPGGSLRLSCA |
| Full CAR | | VSGFALSNHGMSWVRRAPGKGLEWVSGIVYSGSTYYAASVKGR |
| | | FTISRDNSRNTLYLQMNSLRPEDTAIYYCSAHGGESDVWGQGTT |
| | | VTVSSASGGGGSGGRASGGGGSDIRLTQSPSPLSASVGDRVTITC |
| | | QASEDINKFLNWYHQTPGKAPKLLIYDASTLQTGVPSRFSGSGSG |
| | | TDFTLTINSLQPEDIGTYYCQQYESLPLTFGGGTKVEIKTTTPAPR |
| | | PPTPAPTIASQPLSLRPEACRPAAGGAVHTRGLDFACDIYIWAPL |
| | | AGTCGVLLLSLVITLYCKRGRKKLLYIFKQPFMRPVQTTQEEDG |
| | | CSCRFPEEEEGGCELRVKFSRSADAPAYKQGQNQLYNELNLGRR |
| | | EEYDVLDKRRGRDPEMGGKPRRKNPQEGLYNELQKDKMAEAY |
| | | SEIGMKGERRRGKGHDGLYQGLSTATKDTYDALHMQALPPR |
| 139112-nt | 1477 | ATGGCCCTCCCTGTCACCGCCCTGCTGCTTCCGCTGGCTCTTC |
| Full CAR | | TGCTCCACGCCGCTCGGCCCCAAGTGCAACTCGTGGAATCTG |
| | | GTGGAGGACTCGTGCAACCCGGTGGAAGCCTTAGGCTGTCGT |
| | | GCGCCGTCAGCGGGTTTGCTCTGAGCAACCATGGAATGTCCT |
| | | GGGTCCGCCGGGCACCGGGAAAAGGGCTGGAATGGGTGTCC |
| | | GGCATCGTGTACAGCGGGTCAACCTATTACGCCGCGTCCGTG |
| | | AAGGGCAGATTCACTATCTCAAGAGACAACAGCCGGAACACC |
| | | CTGTACTTGCAAATGAATTCCCTGCGCCCCGAGGACACCGCC |
| | | ATCTACTACTGCTCCGCCCACGGAGGAGAGTCGGACGTGTGG |
| | | GGCCAGGGAACGACTGTGACTGTGTCCAGCGCATCAGGAGGG |
| | | GGTGGTTCGGGCGGCCGGGCCTCGGGGGGAGGAGGTTCCGAC |
| | | ATTCGGCTGACCCAGTCCCCGTCCCCACTGTCGGCCTCCGTCG |
| | | GCGACCGCGTGACCATCACTTGTCAGGCGTCCGAGGACATTA |
| | | ACAAGTTCCTGAACTGGTACCACCAGACCCCTGGAAAGGCCC |
| | | CCAAGCTGCTGATCTACGATGCCTCGACCCTTCAAACTGGAGT |
| | | GCCTAGCCGGTTCTCCGGGTCCGGCTCCGGCACTGATTTCACT |
| | | CTGACCATCAACTCATTGCAGCCGGAAGATATCGGGACCTAC |
| | | TATTGCCAGCAGTACGAATCCCTCCCGCTCACATTCGGCGGG |
| | | GGAACCAAGGTCGAGATTAAGACCACTACCCCAGCACCGAGG |
| | | CCACCCACCCCGGCTCCTACCATCGCCTCCCAGCCTCTGTCCC |
| | | TGCGTCCGGAGGCATGTAGACCCGCAGCTGGTGGGGCCGTGC |
| | | ATACCCGGGGTCTTGACTTCGCCTGCGATATCTACATTTGGGC |
| | | CCCTCTGGCTGGTACTTGCGGGGTCCTGCTGCTTTCACTCGTG |
| | | ATCACTCTTTACTGTAAGCGCGGTCGGAAGAAGCTGCTGTAC |
| | | ATCTTTAAGCAACCCTTCATGAGGCCTGTGCAGACTACTCAAG |
| | | AGGAGGACGGCTGTTCATGCCGGTTCCCAGAGGAGGAGGAA |
| | | GGCGGCTGCGAACTGCGCGTGAAATTCAGCCGCAGCGCAGAT |
| | | GCTCCAGCCTACAAGCAGGGGCAGAACCAGCTCTACAACGAA |
| | | CTCAATCTTGGTCGGAGAGAGGAGTACGACGTGCTGGACAAG |
| | | CGGAGAGGACGGGACCCAGAAATGGGCGGGAAGCCGCGCAG |
| | | AAAGAATCCCCAAGAGGGCCTGTACAACGAGCTCCAAAAGG |
| | | ATAAGATGGCAGAAGCCTATAGCGAGATTGGTATGAAAGGG |
| | | GAACGCAGAAGAGGCAAAGGCCACGACGGACTGTACCAGGG |
| | | ACTCAGCACCGCCACCAAGGACACCTATGACGCTCTTCACAT |
| | | GCAGGCCCTGCCGCCTCGG |

139113

| | | |
|---|---|---|
| 139113-aa | 1478 | EVQLVETGGGLVQPGGSLRLSCAVSGFALSNHGMSWVRRAPGK |
| ScFv | | GLEWVSGIVYSGSTYYAASVKGRFTISRDNSRNTLYLQMNSLRP |
| domain | | EDTAIYYCSAHGGESDVWGQGTTVTVSSASGGGGSGGRASGGG |
| | | GSETTLTQSPATLSVSPGERATLSCRASQSVGSNLAWYQQKPGQ |
| | | GPRLLIYGASTRATGIPARFSGSGSGTEFTLTISSLQPEDFAVYYC |
| | | QQYNDWLPVTFGQGTKVEIK |
| 139113-nt | 1480 | GAAGTGCAATTGGTGGAAACTGGAGGAGGACTTGTGCAACCT |
| ScFv | | GGAGGATCATTGCGGCTCTCATGCGCTGTCTCCGGCTTCGCCC |
| domain | | TGTCAAATCACGGGATGTCGTGGGTCAGACGGGCCCCGGGAA |
| | | AGGGTCTGGAATGGGTGTCGGGGATTGTGTACAGCGGCTCCA |
| | | CCTACTACGCCGCTTCGGTCAAGGGCCGCTTCACTATTTCACG |
| | | GGACAACAGCCGCAACACCCTCTATCTGCAAATGAACTCTCT |
| | | CCGCCCGGAGGATACCGCCATCTACTACTGCTCCGCACACGG |
| | | CGGCGAATCCGACGTGTGGGGACAGGGAACCACTGTCACCGT |
| | | GTCGTCCGCATCCGGTGGCGGAGGATCGGGTGGCCGGGCCTC |
| | | CGGGGGCGGCGGCAGCGAGACTACCCTGACCCAGTCCCCTGC |
| | | CACTCTGTCCGTGAGCCCGGGAGAGAGAGCCACCCTTAGCTG |
| | | CCGGGCCAGCCAGAGCGTGGGCTCCAACCTGGCCTGGTACCA |
| | | GCAGAAGCCAGGACAGGGTCCCAGGCTGCTGATCTACGGAGC |
| | | CTCCACTCGCGCGACCGGCATCCCCGCGAGGTTCTCCGGGTC |
| | | GGGTTCCGGGACCGAGTTCACCCTGACCATCTCCTCCCTCCAA |
| | | CCGGAGGACTTCGCGGTGTACTACTGTCAGCAGTACAACGAT |
| | | TGGCTGCCCGTGACATTTGGACAGGGGACGAAGGTGGAAATC |
| | | AAA |
| 139113-aa | 1481 | EVQLVETGGGLVQPGGSLRLSCAVSGFALSNHGMSWVRRAPGK |
| VH | | GLEWVSGIVYSGSTYYAASVKGRFTISRDNSRNTLYLQMNSLRP |
| | | EDTAIYYCSAHGGESDVWGQGTTVTVSS |
| 139113-aa | 1482 | ETTLTQSPATLSVSPGERATLSCRASQSVGSNLAWYQQKPGQGP |
| VL | | RLLIYGASTRATGIPARFSGSGSGTEFTLTISSLQPEDFAVYYCQQ |
| | | YNDWLPVTFGQGTKVEIK |
| 139113-aa | 1483 | MALPVTALLLPLALLLHAARPEVQLVETGGGLVQPGGSLRLSCA |
| Full CAR | | VSGFALSNHGMSWVRRAPGKGLEWVSGIVYSGSTYYAASVKGR |
| | | FTISRDNSRNTLYLQMNSLRPEDTAIYYCSAHGGESDVWGQGTT |
| | | VTVSSASGGGGSGGRASGGGGSETTLTQSPATLSVSPGERATLSC |
| | | RASQSVGSNLAWYQQKPGQGPRLLIYGASTRATGIPARFSGSGS |
| | | GTEFTLTISSLQPEDFAVYYCQQYNDWLPVTFGQGTKVEIKTTTP |
| | | APRPPTPAPTIASQPLSLRPEACRPAAGGAVHTRGLDFACDIYIW |
| | | APLAGTCGVLLLSLVITLYCKRGRKKLLYIFKQPFMRPVQTTQEE |
| | | DGCSCRFPEEEEGGCELRVKFSRSADAPAYKQGQNQLYNELNLG |
| | | RREEYDVLDKRRGRDPEMGGKPRRKNPQEGLYNELQKDKMAE |
| | | AYSEIGMKGERRRGKGHDGLYQGLSTATKDTYDALHMQALPPR |
| 139113-nt | 1484 | ATGGCCCTCCCTGTCACCGCCCTGCTGCTTCCGCTGGCTCTTC |
| Full CAR | | TGCTCCACGCCGCTCGGCCCGAAGTGCAATTGGTGGAAACTG |
| | | GAGGAGGACTTGTGCAACCTGGAGGATCATTGCGGCTCTCAT |
| | | GCGCTGTCTCCGGCTTCGCCCTGTCAAATCACGGGATGTCGTG |
| | | GGTCAGACGGGCCCCGGGAAAGGGTCTGGAATGGGTGTCGG |
| | | GGATTGTGTACAGCGGCTCCACCTACTACGCCGCTTCGGTCAA |
| | | GGGCCGCTTCACTATTTCACGGGACAACAGCCGCAACACCCT |
| | | CTATCTGCAAATGAACTCTCTCCGCCCGGAGGATACCGCCATC |
| | | TACTACTGCTCCGCACACGGCGGCGAATCCGACGTGTGGGGA |
| | | CAGGGAACCACTGTCACCGTGTCGTCCGCATCCGGTGGCGGA |
| | | GGATCGGGTGGCCGGGCCTCCGGGGGCGGCGGCAGCGAGAC |
| | | TACCCTGACCCAGTCCCCTGCCACTCTGTCCGTGAGCCCGGGA |
| | | GAGAGAGCCACCCTTAGCTGCCGGGCCAGCCAGAGCGTGGGC |
| | | TCCAACCTGGCCTGGTACCAGCAGAAGCCAGGACAGGGTCCC |
| | | AGGCTGCTGATCTACGGAGCCTCCACTCGCGCGACCGGCATC |
| | | CCCGCGAGGTTCTCCGGGTCGGGTTCCGGGACCGAGTTCACC |
| | | CTGACCATCTCCTCCCTCCAACCGGAGGACTTCGCGGTGTACT |
| | | ACTGTCAGCAGTACAACGATTGGCTGCCCGTGACATTTGGAC |
| | | AGGGGACGAAGGTGGAAATCAAAACCACTACCCCAGCACCG |
| | | AGGCCACCCACCCCGGCTCCTACCATCGCCTCCCAGCCTCTGT |
| | | CCCTGCGTCCGGAGGCATGTAGACCCGCAGCTGGTGGGGCCG |
| | | TGCATACCCGGGGTCTTGACTTCGCCTGCGATATCTACATTTG |
| | | GGCCCCTCTGGCTGGTACTTGCGGGGTCCTGCTGCTTTCACTC |
| | | GTGATCACTCTTTACTGTAAGCGCGGTCGGAAGAAGCTGCTG |
| | | TACATCTTTAAGCAACCCTTCATGAGGCCTGTGCAGACTACTC |
| | | AAGAGGAGGACGGCTGTTCATGCCGGTTCCCAGAGGAGGAG |
| | | GAAGGCGGCTGCGAACTGCGCGTGAAATTCAGCCGCAGCGCA |
| | | GATGCTCCAGCCTACAAGCAGGGGCAGAACCAGCTCTACAAC |
| | | GAACTCAATCTTGGTCGGAGAGAGGAGTACGACGTGCTGGAC |
| | | AAGCGGAGAGGACGGGACCCAGAAATGGGCGGGAAGCCGCG |
| | | CAGAAAGAATCCCCAAGAGGGCCTGTACAACGAGCTCCAAA |
| | | AGGATAAGATGGCAGAAGCCTATAGCGAGATTGGTATGAAA |
| | | GGGGAACGCAGAAGAGGCAAAGGCCACGACGGACTGTACCA |
| | | GGGACTCAGCACCGCCACCAAGGACACCTATGACGCTCTTCA |
| | | CATGCAGGCCCTGCCGCCTCGG |

139114

| | | |
|---|---|---|
| 139114-aa | 1485 | EVQLVESGGGLVQPGGSLRLSCAVSGFALSNHGMSWVRRAPGK |
| ScFv | | GLEWVSGIVYSGSTYYAASVKGRFTISRDNSRNTLYLQMNSLRP |
| domain | | EDTAIYYCSAHGGESDVWGQGTTVTVSSASGGGGSGGRASGGG |
| | | GSEIVLTQSPGTLSLSPGERATLSCRASQSIGSSSLAWYQQKPGQ |
| | | APRLLMYGASSRASGIPDRFSGSGSGTDFTLTISRLEPEDFAVYYC |
| | | QQYAGSPPFTFGQGTKVEIK |
| 139114-nt | 1486 | GAAGTGCAATTGGTGGAATCTGGTGGAGGACTTGTGCAACCT |
| ScFv | | GGAGGATCACTGAGACTGTCATGCGCGGTGTCCGGTTTTGCC |
| domain | | CTGAGCAATCATGGGATGTCGTGGGTCCGGCGCGCCCCCGGA |
| | | AAGGGTCTGGAATGGGTGTCGGGTATCGTCTACTCCGGGAGC |
| | | ACTTACTACGCCGCGAGCGTGAAGGGCCGCTTCACCATTTCCC |
| | | GCGATAACTCCCGCAACACCCTGTACTTGCAAATGAACTCGC |
| | | TCCGGCCTGAGGACACTGCCATCTACTACTGCTCCGCACACG |
| | | GAGGAGAATCCGACGTGTGGGGCCAGGGAACTACCGTGACC |
| | | GTCAGCAGCGCCTCCGGCGGCGGGGGCTCAGGCGGACGGGCT |
| | | AGCGGCGGCGGTGGCTCCGAGATCGTGCTGACCCAGTCGCCT |
| | | GGCACTCTCTCGCTGAGCCCCGGGGAAAGGGCAACCCTGTCC |
| | | TGTCGGGCCAGCCAGTCCATTGGATCATCCTCCCTCGCCTGGT |
| | | ATCAGCAGAAACCGGGACAGGCTCCGCGGCTGCTTATGTATG |
| | | GGGCCAGCTCAAGAGCCTCCGGCATTCCCGACCGGTTCTCCG |
| | | GGTCCGGTTCCGGCACCGATTTCACCCTGACTATCTCGAGGCT |
| | | GGAGCCAGAGGACTTCGCCGTGTACTACTGCCAGCAGTACGC |
| | | GGGGTCCCCGCCGTTCACGTTCGGACAGGGAACCAAGGTCGA |
| | | GATCAAG |
| 139114-aa | 1487 | EVQLVESGGGLVQPGGSLRLSCAVSGFALSNHGMSWVRRAPGK |
| VH | | GLEWVSGIVYSGSTYYAASVKGRFTISRDNSRNTLYLQMNSLRP |
| | | EDTAIYYCSAHGGESDVWGQGTTVTVSS |
| 139114-aa | 1488 | EIVLTQSPGTLSLSPGERATLSCRASQSIGSSSLAWYQQKPGQAPR |
| VL | | LLMYGASSRASGIPDRFSGSGSGTDFTLTISRLEPEDFAVYYCQQ |
| | | YAGSPPFTFGQGTKVEIK |
| 139114-aa | 1489 | MALPVTALLLPLALLLHAARPEVQLVESGGGLVQPGGSLRLSCA |
| Full CAR | | VSGFALSNHGMSWVRRAPGKGLEWVSGIVYSGSTYYAASVKGR |
| | | FTISRDNSRNTLYLQMNSLRPEDTAIYYCSAHGGESDVWGQGTT |
| | | VTVSSASGGGGSGGRASGGGGSEIVLTQSPGTLSLSPGERATLSC |
| | | RASQSIGSSSLAWYQQKPGQAPRLLMYGASSRASGIPDRFSGSGS |
| | | GTDFTLTISRLEPEDFAVYYCQQYAGSPPFTFGQGTKVEIKTTTP |
| | | APRPPTPAPTIASQPLSLRPEACRPAAGGAVHTRGLDFACDIYIW |
| | | APLAGTCGVLLLSLVITLYCKRGRKKLLYIFKQPFMRPVQTTQEE |
| | | DGCSCRFPEEEEGGCELRVKFSRSADAPAYKQGQNQLYNELNLG |
| | | RREEYDVLDKRRGRDPEMGGKPRRKNPQEGLYNELQKDKMAE |
| | | AYSEIGMKGERRRGKGHDGLYQGLSTATKDTYDALHMQALPPR |
| 139114-nt | 1490 | ATGGCCCTCCCTGTCACCGCCCTGCTGCTTCCGCTGGCTCTTC |
| Full CAR | | TGCTCCACGCCGCTCGGCCCGAAGTGCAATTGGTGGAATCTG |
| | | GTGGAGGACTTGTGCAACCTGGAGGATCACTGAGACTGTCAT |
| | | GCGCGGTGTCCGGTTTTGCCCTGAGCAATCATGGGATGTCGTG |
| | | GGTCCGGCGCGCCCCCGGAAAGGGTCTGGAATGGGTGTCGGG |
| | | TATCGTCTACTCCGGGAGCACTTACTACGCCGCGAGCGTGAA |
| | | GGGCCGCTTCACCATTTCCCGCGATAACTCCCGCAACACCCTG |
| | | TACTTGCAAATGAACTCGCTCCGGCCTGAGGACACTGCCATCT |
| | | ACTACTGCTCCGCACACGGAGGAGAATCCGACGTGTGGGGCC |
| | | AGGGAACTACCGTGACCGTCAGCAGCGCCTCCGGCGGCGGGG |
| | | GCTCAGGCGGACGGGCTAGCGGCGGCGGTGGCTCCGAGATCG |
| | | TGCTGACCCAGTCGCCTGGCACTCTCTCGCTGAGCCCCGGGG |
| | | AAAGGGCAACCCTGTCCTGTCGGGCCAGCCAGTCCATTGGAT |
| | | CATCCTCCCTCGCCTGGTATCAGCAGAAACCGGGACAGGCTC |
| | | CGCGGCTGCTTATGTATGGGGCCAGCTCAAGAGCCTCCGGCA |
| | | TTCCCGACCGGTTCTCCGGGTCCGGTTCCGGCACCGATTTCAC |
| | | CCTGACTATCTCGAGGCTGGAGCCAGAGGACTTCGCCGTGTA |
| | | CTACTGCCAGCAGTACGCGGGGTCCCCGCCGTTCACGTTCGG |
| | | ACAGGGAACCAAGGTCGAGATCAAGACCACTACCCCAGCACC |
| | | GAGGCCACCCACCCCGGCTCCTACCATCGCCTCCCAGCCTCTG |
| | | TCCCTGCGTCCGGAGGCATGTAGACCCGCAGCTGGTGGGGCC |
| | | GTGCATACCCGGGGTCTTGACTTCGCCTGCGATATCTACATTT |
| | | GGGCCCCTCTGGCTGGTACTTGCGGGGTCCTGCTGCTTTCACT |
| | | CGTGATCACTCTTTACTGTAAGCGCGGTCGGAAGAAGCTGCT |
| | | GTACATCTTTAAGCAACCCTTCATGAGGCCTGTGCAGACTACT |
| | | CAAGAGGAGGACGGCTGTTCATGCCGGTTCCCAGAGGAGGAG |
| | | GAAGGCGGCTGCGAACTGCGCGTGAAATTCAGCCGCAGCGCA |
| | | GATGCTCCAGCCTACAAGCAGGGGCAGAACCAGCTCTACAAC |
| | | GAACTCAATCTTGGTCGGAGAGAGGAGTACGACGTGCTGGAC |
| | | AAGCGGAGAGGACGGGACCCAGAAATGGGCGGGAAGCCGCG |
| | | CAGAAAGAATCCCCAAGAGGGCCTGTACAACGAGCTCCAAA |
| | | AGGATAAGATGGCAGAAGCCTATAGCGAGATTGGTATGAAA |
| | | GGGGAACGCAGAAGAGGCAAAGGCCACGACGGACTGTACCA |
| | | GGGACTCAGCACCGCCACCAAGGACACCTATGACGCTCTTCA |
| | | CATGCAGGCCCTGCCGCCTCGG |

149362

| | | |
|---|---|---|
| 149362-aa | 1491 | QVQLQESGPGLVKPSETLSLTCTVSGGSISSSYYYWGWIRQPPGK |
| ScFv | | GLEWIGSIYYSGSAYYNPSLKSRVTISVDTSKNQFSLRLSSVTAA |
| domain | | DTAVYYCARHWQEWPDAFDIWGQGTMVTVSSGGGGSGGGGS |
| | | GGGGSETTLTQSPAFMSATPGDKVIISCKASQDIDDAMNWYQQK |
| | | PGEAPLFIIQSATSPVPGIPPRFSGSGFGTDFSLTINNIESEDAAYYF |
| | | CLQHDNFPLTFGQGTKLEIK |
| 149362-nt | 1492 | CAAGTGCAGCTTCAGGAAAGCGGACCGGGCCTGGTCAAGCCA |
| ScFv | | TCCGAAACTCTCTCCCTGACTTGCACTGTGTCTGGCGGTTCCA |
| domain | | TCTCATCGTCGTACTACTACTGGGGCTGGATTAGGCAGCCGCC |
| | | CGGAAAGGGACTGGAGTGGATCGGAAGCATCTACTATTCCGG |
| | | CTCGGCGTACTACAACCCTAGCCTCAAGTCGAGAGTGACCAT |
| | | CTCCGTGGATACCTCCAAGAACCAGTTTTCCCTGCGCCTGAGC |
| | | TCCGTGACCGCCGCTGACACCGCCGTGTACTACTGTGCTCGGC |
| | | ATTGGCAGGAATGGCCCGATGCCTTCGACATTTGGGGCCAGG |
| | | GCACTATGGTCACTGTGTCATCCGGGGGTGGAGGCAGCGGGG |
| | | GAGGAGGGTCCGGGGGGGGAGGTTCAGAGACAACCTTGACC |
| | | CAGTCACCCGCATTCATGTCCGCCACTCCGGGAGACAAGGTC |
| | | ATCATCTCGTGCAAAGCGTCCCAGGATATCGACGATGCCATG |
| | | AATTGGTACCAGCAGAAGCCTGGCGAAGCGCCGCTGTTCATT |
| | | ATCCAATCCGCAACCTCGCCCGTGCCTGGAATCCCACCGCGG |
| | | TTCAGCGGCAGCGGTTTCGGAACCGACTTTTCCCTGACCATTA |
| | | ACAACATTGAGTCCGAGGACGCCGCCTACTACTTCTGCCTGC |
| | | AACACGACAACTTCCCTCTCACGTTCGGCCAGGGAACCAAGC |
| | | TGGAAATCAAG |
| 149362-aa | 1493 | QVQLQESGPGLVKPSETLSLTCTVSGGSISSSYYYWGWIRQPPGK |
| VH | | GLEWIGSIYYSGSAYYNPSLKSRVTISVDTSKNQFSLRLSSVTAA |
| | | DTAVYYCARHWQEWPDAFDIWGQGTMVTVSS |
| 149362-aa | 1494 | ETTLTQSPAFMSATPGDKVIISCKASQDIDDAMNWYQQKPGEAP |
| VL | | LFIIQSATSPVPGIPPRFSGSGFGTDFSLTINNIESEDAAYYFCLQH |
| | | DNFPLTFGQGTKLEIK |
| 149362-aa | 1495 | MALPVTALLLPLALLLHAARPQVQLQESGPGLVKPSETLSLTCT |
| Full CAR | | VSGGSISSSYYYWGWIRQPPGKGLEWIGSIYYSGSAYYNPSLKSR |
| | | VTISVDTSKNQFSLRLSSVTAADTAVYYCARHWQEWPDAFDIW |
| | | GQGTMVTVSSGGGGSGGGGSGGGGSETTLTQSPAFMSATPGDK |
| | | VIISCKASQDIDDAMNWYQQKPGEAPLFIIQSATSPVPGIPPRFSG |
| | | SGFGTDFSLTINNIESEDAAYYFCLQHDNFPLTFGQGTKLEIKTTT |
| | | PAPRPPTPAPTIASQPLSLRPEACRPAAGGAVHTRGLDFACDIYIW |
| | | APLAGTCGVLLLSLVITLYCKRGRKKLLYIFKQPFMRPVQTTQEE |
| | | DGCSCRFPEEEEGGCELRVKFSRSADAPAYKQGQNQLYNELNLG |
| | | RREEYDVLDKRRGRDPEMGGKPRRKNPQEGLYNELQKDKMAE |
| | | AYSEIGMKGERRRGKGHDGLYQGLSTATKDTYDALHMQALPPR |
| 149362-nt | 1496 | ATGGCCCTCCCTGTCACCGCCCTGCTGCTTCCGCTGGCTCTTC |
| Full CAR | | TGCTCCACGCCGCTCGGCCCCAAGTGCAGCTTCAGGAAAGCG |
| | | GACCGGGCCTGGTCAAGCCATCCGAAACTCTCTCCCTGACTTG |
| | | CACTGTGTCTGGCGGTTCCATCTCATCGTCGTACTACTACTGG |
| | | GGCTGGATTAGGCAGCCGCCCGGAAAGGGACTGGAGTGGATC |
| | | GGAAGCATCTACTATTCCGGCTCGGCGTACTACAACCCTAGC |
| | | CTCAAGTCGAGAGTGACCATCTCCGTGGATACCTCCAAGAAC |
| | | CAGTTTTCCCTGCGCCTGAGCTCCGTGACCGCCGCTGACACCG |
| | | CCGTGTACTACTGTGCTCGGCATTGGCAGGAATGGCCCGATG |
| | | CCTTCGACATTTGGGGCCAGGGCACTATGGTCACTGTGTCATC |
| | | CGGGGGTGGAGGCAGCGGGGGAGGAGGGTCCGGGGGGGGAG |
| | | GTTCAGAGACAACCTTGACCCAGTCACCCGCATTCATGTCCGC |
| | | CACTCCGGGAGACAAGGTCATCATCTCGTGCAAAGCGTCCCA |
| | | GGATATCGACGATGCCATGAATTGGTACCAGCAGAAGCCTGG |
| | | CGAAGCGCCGCTGTTCATTATCCAATCCGCAACCTCGCCCGTG |
| | | CCTGGAATCCCACCGCGGTTCAGCGGCAGCGGTTTCGGAACC |
| | | GACTTTTCCCTGACCATTAACAACATTGAGTCCGAGGACGCC |
| | | GCCTACTACTTCTGCCTGCAACACGACAACTTCCCTCTCACGT |
| | | TCGGCCAGGGAACCAAGCTGGAAATCAAGACCACTACCCCAG |
| | | CACCGAGGCCACCCACCCCGGCTCCTACCATCGCCTCCCAGC |
| | | CTCTGTCCCTGCGTCCGGAGGCATGTAGACCCGCAGCTGGTG |
| | | GGGCCGTGCATACCCGGGGTCTTGACTTCGCCTGCGATATCTA |
| | | CATTTGGGCCCCTCTGGCTGGTACTTGCGGGGTCCTGCTGCTT |
| | | TCACTCGTGATCACTCTTTACTGTAAGCGCGGTCGGAAGAAG |
| | | CTGCTGTACATCTTTAAGCAACCCTTCATGAGGCCTGTGCAGA |
| | | CTACTCAAGAGGAGGACGGCTGTTCATGCCGGTTCCCAGAGG |
| | | AGGAGGAAGGCGGCTGCGAACTGCGCGTGAAATTCAGCCGC |
| | | AGCGCAGATGCTCCAGCCTACAAGCAGGGGCAGAACCAGCTC |
| | | TACAACGAACTCAATCTTGGTCGGAGAGAGGAGTACGACGTG |
| | | CTGGACAAGCGGAGAGGACGGGACCCAGAAATGGGCGGGAA |
| | | GCCGCGCAGAAAGAATCCCCAAGAGGGCCTGTACAACGAGCT |
| | | CCAAAAGGATAAGATGGCAGAAGCCTATAGCGAGATTGGTAT |
| | | GAAAGGGGAACGCAGAAGAGGCAAAGGCCACGACGGACTGT |
| | | ACCAGGGACTCAGCACCGCCACCAAGGACACCTATGACGCTC |
| | | TTCACATGCAGGCCCTGCCGCCTCGG |

149363

| | | |
|---|---|---|
| 149363-aa | 1497 | VNLRESGPALVKPTQTLTLTCTFSGFSLRTSGMCVSWIRQPPGKA |
| ScFv | | LEWLARIDWDEDKFYSTSLKTRLTISKDTSDNQVVLRMTNMDP |
| domain | | ADTATYYCARSGAGGTSATAFDIWGPGTMVTVSSGGGGSGGGG |
| | | SGGGGSDIQMTQSPSSLSASVGDRVTITCRASQDIYNNLAWFQL |
| | | KPGSAPRSLMYAANKSQSGVPSRFSGSASGTDFTLTISSLQPEDF |
| | | ATYYCQHYYRFPYSFGQGTKLEIK |
| 149363-nt | 1498 | CAAGTCAATCTGCGCGAATCCGGCCCCGCCTTGGTCAAGCCT |
| ScFv | | ACCCAGACCCTCACTCTGACCTGTACTTTCTCCGGCTTCTCCC |
| domain | | TGCGGACTTCCGGGATGTGCGTGTCCTGGATCAGACAGCCTC |
| | | CGGGAAAGGCCCTGGAGTGGCTCGCTCGCATTGACTGGGATG |
| | | AGGACAAGTTCTACTCCACCTCACTCAAGACCAGGCTGACCA |
| | | TCAGCAAAGATACCTCTGACAACCAAGTGGTGCTCCGCATGA |
| | | CCAACATGGACCCAGCCGACACTGCCACTTACTACTGCGCGA |
| | | GGAGCGGAGCGGGCGGAACCTCCGCCACCGCCTTCGATATTT |
| | | GGGGCCCGGGTACCATGGTCACCGTGTCAAGCGGAGGAGGG |
| | | GGGTCCGGGGGCGGCGGTTCCGGGGGAGGCGGATCGGACATT |
| | | CAGATGACTCAGTCACCATCGTCCCTGAGCGCTAGCGTGGGC |
| | | GACAGAGTGACAATCACTTGCCGGGCATCCCAGGACATCTAT |
| | | AACAACCTTGCGTGGTTCCAGCTGAAGCCTGGTTCCGCACCG |
| | | CGGTCACTTATGTACGCCGCCAACAAGAGCCAGTCGGGAGTG |
| | | CCGTCCCGGTTTTCCGGTTCGGCCTCGGGAACTGACTTCACCC |
| | | TGACGATCTCCAGCCTGCAACCCGAGGATTTCGCCACCTACTA |
| | | CTGCCAGCACTACTACCGCTTTCCCTACTCGTTCGGACAGGGA |
| | | ACCAAGCTGGAAATCAAG |
| 149363-aa | 1499 | QVNLRESGPALVKPTQTLTLTCTFSGFSLRTSGMCVSWIRQPPGK |
| VH | | ALEWLARIDWDEDKFYSTSLKTRLTISKDTSDNQVVLRMTNMD |
| | | PADTATYYCARSGAGGTSATAFDIWGPGTMVTVSS |
| 149363-aa | 1500 | DIQMTQSPSSLSASVGDRVTITCRASQDIYNNLAWFQLKPGSAPR |
| VL | | SLMYAANKSQSGVPSRFSGSASGTDFTLTISSLQPEDFATYYCQH |
| | | YYRFPYSFGQGTKLEIK |
| 149363-aa | 1501 | MALPVTALLLPLALLLHAARPQVNLRESGPALVKPTQTLTLTCT |
| Full CAR | | FSGFSLRTSGMCVSWIRQPPGKALEWLARIDWDEDKFYSTSLKT |
| | | RLTISKDTSDNQVVLRMTNMDPADTATYYCARSGAGGTSATAF |
| | | DIWGPGTMVTVSSGGGGSGGGGSGGGGSDIQMTQSPSSLSASVG |
| | | DRVTITCRASQDIYNNLAWFQLKPGSAPRSLMYAANKSQSGVPS |
| | | RFSGSASGTDFTLTISSLQPEDFATYYCQHYYRFPYSFGQGTKLEI |
| | | KTTTPAPRPPTPAPTIASQPLSLRPEACRPAAGGAVHTRGLDFAC |
| | | DIYIWAPLAGTCGVLLLSLVITLYCKRGRKKLLYIFKQPFMRPVQ |
| | | TTQEEDGCSCRFPEEEEGGCELRVKFSRSADAPAYKQGQNQLYN |
| | | ELNLGRREEYDVLDKRRGRDPEMGGKPRRKNPQEGLYNELQKD |
| | | KMAEAYSEIGMKGERRRGKGHDGLYQGLSTATKDTYDALHMQ |
| | | ALPPR |
| 149363-nt | 1502 | ATGGCCCTCCCTGTCACCGCCCTGCTGCTTCCGCTGGCTCTTC |
| Full CAR | | TGCTCCACGCCGCTCGGCCCCAAGTCAATCTGCGCGAATCCG |
| | | GCCCCGCCTTGGTCAAGCCTACCCAGACCCTCACTCTGACCTG |
| | | TACTTTCTCCGGCTTCTCCCTGCGGACTTCCGGGATGTGCGTG |
| | | TCCTGGATCAGACAGCCTCCGGGAAAGGCCCTGGAGTGGCTC |
| | | GCTCGCATTGACTGGGATGAGGACAAGTTCTACTCCACCTCA |
| | | CTCAAGACCAGGCTGACCATCAGCAAAGATACCTCTGACAAC |
| | | CAAGTGGTGCTCCGCATGACCAACATGGACCCAGCCGACACT |
| | | GCCACTTACTACTGCGCGAGGAGCGGAGCGGGCGGAACCTCC |
| | | GCCACCGCCTTCGATATTTGGGGCCCGGGTACCATGGTCACC |
| | | GTGTCAAGCGGAGGAGGGGGGTCCGGGGGCGGCGGTTCCGG |
| | | GGGAGGCGGATCGGACATTCAGATGACTCAGTCACCATCGTC |
| | | CCTGAGCGCTAGCGTGGGCGACAGAGTGACAATCACTTGCCG |
| | | GGCATCCCAGGACATCTATAACAACCTTGCGTGGTTCCAGCT |
| | | GAAGCCTGGTTCCGCACCGCGGTCACTTATGTACGCCGCCAA |
| | | CAAGAGCCAGTCGGGAGTGCCGTCCCGGTTTTCCGGTTCGGC |
| | | CTCGGGAACTGACTTCACCCTGACGATCTCCAGCCTGCAACCC |
| | | GAGGATTTCGCCACCTACTACTGCCAGCACTACTACCGCTTTC |
| | | CCTACTCGTTCGGACAGGGAACCAAGCTGGAAATCAAGACCA |
| | | CTACCCCAGCACCGAGGCCACCCACCCCGGCTCCTACCATCG |
| | | CCTCCCAGCCTCTGTCCCTGCGTCCGGAGGCATGTAGACCCGC |
| | | AGCTGGTGGGGCCGTGCATACCCGGGGTCTTGACTTCGCCTG |
| | | CGATATCTACATTTGGGCCCCTCTGGCTGGTACTTGCGGGGTC |
| | | CTGCTGCTTTCACTCGTGATCACTCTTTACTGTAAGCGCGGTC |
| | | GGAAGAAGCTGCTGTACATCTTTAAGCAACCCTTCATGAGGC |
| | | CTGTGCAGACTACTCAAGAGGAGGACGGCTGTTCATGCCGGT |
| | | TCCCAGAGGAGGAGGAAGGCGGCTGCGAACTGCGCGTGAAA |
| | | TTCAGCCGCAGCGCAGATGCTCCAGCCTACAAGCAGGGGCAG |
| | | AACCAGCTCTACAACGAACTCAATCTTGGTCGGAGAGAGGAG |
| | | TACGACGTGCTGGACAAGCGGAGAGGACGGGACCCAGAAAT |
| | | GGGCGGGAAGCCGCGCAGAAAGAATCCCCAAGAGGGCCTGT |
| | | ACAACGAGCTCCAAAAGGATAAGATGGCAGAAGCCTATAGC |
| | | GAGATTGGTATGAAAGGGGAACGCAGAAGAGGCAAAGGCCA |
| | | CGACGGACTGTACCAGGGACTCAGCACCGCCACCAAGGACAC |
| | | CTATGACGCTCTTCACATGCAGGCCCTGCCGCCTCGG |

149364

| | | |
|---|---|---|
| 149364-aa | 1503 | EVQLVESGGGLVKPGGSLRLSCAASGFTFSSYSMNWVRQAPGK |
| ScFv | | GLEWVSSISSSSSYIYYADSVKGRFTISRDNAKNSLYLQMNSLRA |
| domain | | EDTAVYYCAKTIAAVYAFDIWGQGTTVTVSSGGGGSGGGGSGG |
| | | GGSEIVLTQSPLSLPVTPEEPASISCRSSQSLLHSNGYNYLDWYLQ |
| | | KPGQSPQLLIYLGSNRASGVPDRFSGSGSGTDFTLKISRVEAEDV |
| | | GVYYCMQALQTPYTFGQGTKLEIK |
| 149364-nt | 1504 | GAAGTGCAGCTTGTCGAATCCGGGGGGGGACTGGTCAAGCCG |
| ScFv | | GGCGGATCACTGAGACTGTCCTGCGCCGCGAGCGGCTTCACG |
| domain | | TTCTCCTCCTACTCCATGAACTGGGTCCGCCAAGCCCCCGGGA |
| | | AGGGACTGGAATGGGTGTCCTCTATCTCCTCGTCGTCGTCCTA |
| | | CATCTACTACGCCGACTCCGTGAAGGGAAGATTCACCATTTCC |
| | | CGCGACAACGCAAAGAACTCACTGTACTTGCAAATGAACTCA |
| | | CTCCGGGCCGAAGATACTGCTGTGTACTATTGCGCCAAGACT |
| | | ATTGCCGCCGTCTACGCTTTCGACATCTGGGGCCAGGGAACC |
| | | ACCGTGACTGTGTCGTCCGGTGGTGGTGGCTCGGGCGGAGGA |
| | | GGAAGCGGCGGCGGGGGGTCCGAGATTGTGCTGACCCAGTCG |
| | | CCACTGAGCCTCCCTGTGACCCCCGAGGAACCCGCCAGCATC |
| | | AGCTGCCGGTCCAGCCAGTCCCTGCTCCACTCCAACGGATAC |
| | | AATTACCTCGATTGGTACCTTCAGAAGCCTGGACAAAGCCCG |
| | | CAGCTGCTCATCTACTTGGGATCAAACCGCGCGTCAGGAGTG |
| | | CCTGACCGGTTCTCCGGCTCGGGCAGCGGTACCGATTTCACCC |
| | | TGAAAATCTCCAGGGTGGAGGCAGAGGACGTGGGAGTGTATT |
| | | ACTGTATGCAGGCGCTGCAGACTCCGTACACATTTGGGCAGG |
| | | GCACCAAGCTGGAGATCAAG |
| 149364-aa | 1505 | EVQLVESGGGLVKPGGSLRLSCAASGFTFSSYSMNWVRQAPGK |
| VH | | GLEWVSSISSSSSYIYYADSVKGRFTISRDNAKNSLYLQMNSLRA |
| | | EDTAVYYCAKTIAAVYAFDIWGQGTTVTVSS |
| 149364-aa | 1506 | EIVLTQSPLSLPVTPEEPASISCRSSQSLLHSNGYNYLDWYLQKPG |
| VL | | QSPQLLIYLGSNRASGVPDRFSGSGSGTDFTLKISRVEAEDVGVY |
| | | YCMQALQTPYTFGQGTKLEIK |
| 149364-aa | 1507 | MALPVTALLLPLALLLHAARPEVQLVESGGGLVKPGGSLRLSCA |
| Full CAR | | ASGFTFSSYSMNWVRQAPGKGLEWVSSISSSSSYIYYADSVKGR |
| | | FTISRDNAKNSLYLQMNSLRAEDTAVYYCAKTIAAVYAFDIWG |
| | | QGTTVTVSSGGGGSGGGGSGGGGSEIVLTQSPLSLPVTPEEPASIS |
| | | CRSSQSLLHSNGYNYLDWYLQKPGQSPQLLIYLGSNRASGVPDR |
| | | FSGSGSGTDFTLKISRVEAEDVGVYYCMQALQTPYTFGQGTKLE |
| | | IKTTTPAPRPPTPAPTIASQPLSLRPEACRPAAGGAVHTRGLDFAC |
| | | DIYIWAPLAGTCGVLLLSLVITLYCKRGRKKLLYIFKQPFMRPVQ |
| | | TTQEEDGCSCRFPEEEEGGCELRVKFSRSADAPAYKQGQNQLYN |
| | | ELNLGRREEYDVLDKRRGRDPEMGGKPRRKNPQEGLYNELQKD |
| | | KMAEAYSEIGMKGERRRGKGHDGLYQGLSTATKDTYDALHMQ |
| | | ALPPR |
| 149364-nt | 1508 | ATGGCCCTCCCTGTCACCGCCCTGCTGCTTCCGCTGGCTCTTC |
| Full CAR | | TGCTCCACGCCGCTCGGCCCGAAGTGCAGCTTGTCGAATCCG |
| | | GGGGGGGACTGGTCAAGCCGGGCGGATCACTGAGACTGTCCT |
| | | GCGCCGCGAGCGGCTTCACGTTCTCCTCCTACTCCATGAACTG |
| | | GGTCCGCCAAGCCCCCGGGAAGGGACTGGAATGGGTGTCCTC |
| | | TATCTCCTCGTCGTCGTCCTACATCTACTACGCCGACTCCGTG |
| | | AAGGGAAGATTCACCATTTCCCGCGACAACGCAAAGAACTCA |
| | | CTGTACTTGCAAATGAACTCACTCCGGGCCGAAGATACTGCT |
| | | GTGTACTATTGCGCCAAGACTATTGCCGCCGTCTACGCTTTCG |
| | | ACATCTGGGGCCAGGGAACCACCGTGACTGTGTCGTCCGGTG |
| | | GTGGTGGCTCGGGCGGAGGAGGAAGCGGCGGCGGGGGGTCC |
| | | GAGATTGTGCTGACCCAGTCGCCACTGAGCCTCCCTGTGACCC |
| | | CCGAGGAACCCGCCAGCATCAGCTGCCGGTCCAGCCAGTCCC |
| | | TGCTCCACTCCAACGGATACAATTACCTCGATTGGTACCTTCA |
| | | GAAGCCTGGACAAAGCCCGCAGCTGCTCATCTACTTGGGATC |
| | | AAACCGCGCGTCAGGAGTGCCTGACCGGTTCTCCGGCTCGGG |
| | | CAGCGGTACCGATTTCACCCTGAAAATCTCCAGGGTGGAGGC |
| | | AGAGGACGTGGGAGTGTATTACTGTATGCAGGCGCTGCAGAC |
| | | TCCGTACACATTTGGGCAGGGCACCAAGCTGGAGATCAAGAC |
| | | CACTACCCCAGCACCGAGGCCACCCACCCCGGCTCCTACCAT |
| | | CGCCTCCCAGCCTCTGTCCCTGCGTCCGGAGGCATGTAGACCC |
| | | GCAGCTGGTGGGGCCGTGCATACCCGGGGTCTTGACTTCGCC |
| | | TGCGATATCTACATTTGGGCCCCTCTGGCTGGTACTTGCGGGG |
| | | TCCTGCTGCTTTCACTCGTGATCACTCTTTACTGTAAGCGCGG |
| | | TCGGAAGAAGCTGCTGTACATCTTTAAGCAACCCTTCATGAG |
| | | GCCTGTGCAGACTACTCAAGAGGAGGACGGCTGTTCATGCCG |
| | | GTTCCCAGAGGAGGAGGAAGGCGGCTGCGAACTGCGCGTGA |
| | | AATTCAGCCGCAGCGCAGATGCTCCAGCCTACAAGCAGGGGC |
| | | AGAACCAGCTCTACAACGAACTCAATCTTGGTCGGAGAGAGG |
| | | AGTACGACGTGCTGGACAAGCGGAGAGGACGGGACCCAGAA |
| | | ATGGGCGGGAAGCCGCGCAGAAAGAATCCCCAAGAGGGCCT |
| | | GTACAACGAGCTCCAAAAGGATAAGATGGCAGAAGCCTATA |
| | | GCGAGATTGGTATGAAAGGGGAACGCAGAAGAGGCAAAGGC |
| | | CACGACGGACTGTACCAGGGACTCAGCACCGCCACCAAGGAC |
| | | ACCTATGACGCTCTTCACATGCAGGCCCTGCCGCCTCGG |

149365

| | | |
|---|---|---|
| 149365-aa | 1509 | EVQLVESGGGLVKPGGSLRLSCAASGFTFSDYYMSWIRQAPGKG |
| ScFv | | LEWVSYISSSGSTIYYADSVKGRFTISRDNAKNSLYLQMNSLRAE |
| domain | | DTAVYYCARDLRGAFDIWGQGTMVTVSSGGGGSGGGGSGGGG |
| | | SSYVLTQSPSVSAAPGYTATISCGGNNIGTKSVHWYQQKPGQAP |
| | | LLVIRDDSVRPSKIPGRFSGSNSGNMATLTISGVQAGDEADFYCQ |
| | | VWDSDSEHVVFGGGTKLTVL |
| 149365-nt | 1510 | GAAGTCCAGCTCGTGGAGTCCGGCGGAGGCCTTGTGAAGCCT |
| ScFv | | GGAGGTTCGCTGAGACTGTCCTGCGCCGCCTCCGGCTTCACCT |
| domain | | TCTCCGACTACTACATGTCCTGGATCAGACAGGCCCCGGGAA |
| | | AGGGCCTGGAATGGGTGTCCTACATCTCGTCATCGGGCAGCA |
| | | CTATCTACTACGCGGACTCAGTGAAGGGGCGGTTCACCATTTC |
| | | CCGGGATAACGCGAAGAACTCGCTGTATCTGCAAATGAACTC |
| | | ACTGAGGGCCGAGGACACCGCCGTGTACTACTGCGCCCGCGA |
| | | TCTCCGCGGGGCATTTGACATCTGGGGACAGGGAACCATGGT |
| | | CACAGTGTCCAGCGGAGGGGGAGGATCGGGTGGCGGAGGTT |
| | | CCGGGGGTGGAGGCTCCTCCTACGTGCTGACTCAGAGCCCAA |
| | | GCGTCAGCGCTGCGCCCGGTTACACGGCAACCATCTCCTGTG |
| | | GCGGAAACAACATTGGGACCAAGTCTGTGCACTGGTATCAGC |
| | | AGAAGCCGGGCCAAGCTCCCCTGTTGGTGATCCGCGATGACT |
| | | CCGTGCGGCCTAGCAAAATTCCGGGACGGTTCTCCGGCTCCA |
| | | ACAGCGGCAATATGGCCACTCTCACCATCTCGGGAGTGCAGG |
| | | CCGGAGATGAAGCCGACTTCTACTGCCAAGTCTGGGACTCAG |
| | | ACTCCGAGCATGTGGTGTTCGGGGGCGGAACCAAGCTGACTG |
| | | TGCTC |
| 149365-aa | 1511 | EVQLVESGGGLVKPGGSLRLSCAASGFTFSDYYMSWIRQAPGKG |
| VH | | LEWVSYISSSGSTIYYADSVKGRFTISRDNAKNSLYLQMNSLRAE |
| | | DTAVYYCARDLRGAFDIWGQGTMVTVSS |
| 149365-aa | 1512 | SYVLTQSPSVSAAPGYTATISCGGNNIGTKSVHWYQQKPGQAPL |
| VL | | LVIRDDSVRPSKIPGRFSGSNSGNMATLTISGVQAGDEADFYCQV |
| | | WDSDSEHVVFGGGTKLTVL |
| 149365-aa | 1513 | MALPVTALLLPLALLLHAARPEVQLVESGGGLVKPGGSLRLSCA |
| Full CAR | | ASGFTFSDYYMSWIRQAPGKGLEWVSYISSSGSTIYYADSVKGR |
| | | FTISRDNAKNSLYLQMNSLRAEDTAVYYCARDLRGAFDIWGQG |
| | | TMVTVSSGGGGSGGGGSGGGGSSYVLTQSPSVSAAPGYTATISC |
| | | GGNNIGTKSVHWYQQKPGQAPLLVIRDDSVRPSKIPGRFSGSNS |
| | | GNMATLTISGVQAGDEADFYCQVWDSDSEHVVFGGGTKLTVLT |
| | | TTPAPRPPTPAPTIASQPLSLRPEACRPAAGGAVHTRGLDFACDIY |
| | | IWAPLAGTCGVLLLSLVITLYCKRGRKKLLYIFKQPFMRPVQTTQ |
| | | EEDGCSCRFPEEEEGGCELRVKFSRSADAPAYKQGQNQLYNELN |
| | | LGRREEYDVLDKRRGRDPEMGGKPRRKNPQEGLYNELQKDKM |
| | | AEAYSEIGMKGERRRGKGHDGLYQGLSTATKDTYDALHMQAL |
| | | PPR |
| 149365-nt | 1514 | ATGGCCCTCCCTGTCACCGCCCTGCTGCTTCCGCTGGCTCTTC |
| Full CAR | | TGCTCCACGCCGCTCGGCCCGAAGTCCAGCTCGTGGAGTCCG |
| | | GCGGAGGCCTTGTGAAGCCTGGAGGTTCGCTGAGACTGTCCT |
| | | GCGCCGCCTCCGGCTTCACCTTCTCCGACTACTACATGTCCTG |
| | | GATCAGACAGGCCCCGGGAAAGGGCCTGGAATGGGTGTCCTA |
| | | CATCTCGTCATCGGGCAGCACTATCTACTACGCGGACTCAGTG |
| | | AAGGGGCGGTTCACCATTTCCCGGGATAACGCGAAGAACTCG |
| | | CTGTATCTGCAAATGAACTCACTGAGGGCCGAGGACACCGCC |
| | | GTGTACTACTGCGCCCGCGATCTCCGCGGGGCATTTGACATCT |
| | | GGGGACAGGGAACCATGGTCACAGTGTCCAGCGGAGGGGGA |
| | | GGATCGGGTGGCGGAGGTTCCGGGGGTGGAGGCTCCTCCTAC |
| | | GTGCTGACTCAGAGCCCAAGCGTCAGCGCTGCGCCCGGTTAC |
| | | ACGGCAACCATCTCCTGTGGCGGAAACAACATTGGGACCAAG |
| | | TCTGTGCACTGGTATCAGCAGAAGCCGGGCCAAGCTCCCCTG |
| | | TTGGTGATCCGCGATGACTCCGTGCGGCCTAGCAAAATTCCG |
| | | GGACGGTTCTCCGGCTCCAACAGCGGCAATATGGCCACTCTC |
| | | ACCATCTCGGGAGTGCAGGCCGGAGATGAAGCCGACTTCTAC |
| | | TGCCAAGTCTGGGACTCAGACTCCGAGCATGTGGTGTTCGGG |
| | | GGCGGAACCAAGCTGACTGTGCTCACCACTACCCCAGCACCG |
| | | AGGCCACCCACCCCGGCTCCTACCATCGCCTCCCAGCCTCTGT |
| | | CCCTGCGTCCGGAGGCATGTAGACCCGCAGCTGGTGGGGCCG |
| | | TGCATACCCGGGGTCTTGACTTCGCCTGCGATATCTACATTTG |
| | | GGCCCCTCTGGCTGGTACTTGCGGGGTCCTGCTGCTTTCACTC |
| | | GTGATCACTCTTTACTGTAAGCGCGGTCGGAAGAAGCTGCTG |
| | | TACATCTTTAAGCAACCCTTCATGAGGCCTGTGCAGACTACTC |
| | | AAGAGGAGGACGGCTGTTCATGCCGGTTCCCAGAGGAGGAG |
| | | GAAGGCGGCTGCGAACTGCGCGTGAAATTCAGCCGCAGCGCA |
| | | GATGCTCCAGCCTACAAGCAGGGGCAGAACCAGCTCTACAAC |
| | | GAACTCAATCTTGGTCGGAGAGAGGAGTACGACGTGCTGGAC |
| | | AAGCGGAGAGGACGGGACCCAGAAATGGGCGGGAAGCCGCG |
| | | CAGAAAGAATCCCCAAGAGGGCCTGTACAACGAGCTCCAAA |
| | | AGGATAAGATGGCAGAAGCCTATAGCGAGATTGGTATGAAA |
| | | GGGGAACGCAGAAGAGGCAAAGGCCACGACGGACTGTACCA |
| | | GGGACTCAGCACCGCCACCAAGGACACCTATGACGCTCTTCA |
| | | CATGCAGGCCCTGCCGCCTCGG |

149366

| | | |
|---|---|---|
| 149366-aa | 1515 | QVQLVQSGAEVKKPGASVKVSCKPSGYTVTSHYIHWVRRAPGQ |
| ScFv | | GLEWMGMINPSGGVTAYSQTLQGRVTMTSDTSSSTVYMELSSL |
| domain | | RSEDTAMYYCAREGSGSGWYFDFWGRGTLVTVSSGGGGSGGG |
| | | GSGGGGSSYVLTQPPSVSVSPGQTASITCSGDGLSKKYVSWYQQ |
| | | KAGQSPVVLISRDKERPSGIPDRFSGSNSADTATLTISGTQAMDE |
| | | ADYYCQAWDDTTVVFGGGTKLTVL |
| 149366-nt | 1516 | CAAGTGCAGCTGGTGCAGAGCGGGGCCGAAGTCAAGAAGCC |
| ScFv | | GGGAGCCTCCGTGAAAGTGTCCTGCAAGCCTTCGGGATACAC |
| domain | | CGTGACCTCCCACTACATTCATTGGGTCCGCCGCGCCCCCGGC |
| | | CAAGGACTCGAGTGGATGGGCATGATCAACCCTAGCGGCGGA |
| | | GTGACCGCGTACAGCCAGACGCTGCAGGGACGCGTGACTATG |
| | | ACCTCGGATACCTCCTCCTCCACCGTCTATATGGAACTGTCCA |
| | | GCCTGCGGTCCGAGGATACCGCCATGTACTACTGCGCCCGGG |
| | | AAGGATCAGGCTCCGGGTGGTATTTCGACTTCTGGGGAAGAG |
| | | GCACCCTCGTGACTGTGTCATCTGGGGGAGGGGGTTCCGGTG |
| | | GTGGCGGATCGGGAGGAGGCGGTTCATCCTACGTGCTGACCC |
| | | AGCCACCCTCCGTGTCCGTGAGCCCCGGCCAGACTGCATCGA |
| | | TTACATGTAGCGGCGACGGCCTCTCCAAGAAATACGTGTCGT |
| | | GGTACCAGCAGAAGGCCGGACAGAGCCCGGTGGTGCTGATCT |
| | | CAAGAGATAAGGAGCGGCCTAGCGGAATCCCGGACAGGTTCT |
| | | CGGGTTCCAACTCCGCGGACACTGCTACTCTGACCATCTCGGG |
| | | GACCCAGGCTATGGACGAAGCCGATTACTACTGCCAAGCCTG |
| | | GGACGACACTACTGTCGTGTTTGGAGGGGGCACCAAGTTGAC |
| | | CGTCCTT |
| 149366-aa | 1517 | QVQLVQSGAEVKKPGASVKVSCKPSGYTVTSHYIHWVRRAPGQ |
| VH | | GLEWMGMINPSGGVTAYSQTLQGRVTMTSDTSSSTVYMELSSL |
| | | RSEDTAMYYCAREGSGSGWYFDFWGRGTLVTVSS |
| 149366-aa | 1518 | SYVLTQPPSVSVSPGQTASITCSGDGLSKKYVSWYQQKAGQSPV |
| VL | | VLISRDKERPSGIPDRFSGSNSADTATLTISGTQAMDEADYYCQA |
| | | WDDTTVVFGGGTKLTVL |
| 149366-aa | 1519 | MALPVTALLLPLALLLHAARPQVQLVQSGAEVKKPGASVKVSC |
| Full CAR | | KPSGYTVTSHYIHWVRRAPGQGLEWMGMINPSGGVTAYSQTLQ |
| | | GRVTMTSDTSSSTVYMELSSLRSEDTAMYYCAREGSGSGWYFD |
| | | FWGRGTLVTVSSGGGGSGGGGSGGGGSSYVLTQPPSVSVSPGQT |
| | | ASITCSGDGLSKKYVSWYQQKAGQSPVVLISRDKERPSGIPDRFS |
| | | GSNSADTATLTISGTQAMDEADYYCQAWDDTTVVFGGGTKLTV |
| | | LTTTPAPRPPTPAPTIASQPLSLRPEACRPAAGGAVHTRGLDFAC |
| | | DIYIWAPLAGTCGVLLLSLVITLYCKRGRKKLLYIFKQPFMRPVQ |
| | | TTQEEDGCSCRFPEEEEGGCELRVKFSRSADAPAYKQGQNQLYN |
| | | ELNLGRREEYDVLDKRRGRDPEMGGKPRRKNPQEGLYNELQKD |
| | | KMAEAYSEIGMKGERRRGKGHDGLYQGLSTATKDTYDALHMQ |
| | | ALPPR |
| 149366-nt | 1520 | ATGGCCCTCCCTGTCACCGCCCTGCTGCTTCCGCTGGCTCTTC |
| Full CAR | | TGCTCCACGCCGCTCGGCCCCAAGTGCAGCTGGTGCAGAGCG |
| | | GGGCCGAAGTCAAGAAGCCGGGAGCCTCCGTGAAAGTGTCCT |
| | | GCAAGCCTTCGGGATACACCGTGACCTCCCACTACATTCATTG |
| | | GGTCCGCCGCGCCCCCGGCCAAGGACTCGAGTGGATGGGCAT |
| | | GATCAACCCTAGCGGCGGAGTGACCGCGTACAGCCAGACGCT |
| | | GCAGGGACGCGTGACTATGACCTCGGATACCTCCTCCTCCAC |
| | | CGTCTATATGGAACTGTCCAGCCTGCGGTCCGAGGATACCGC |
| | | CATGTACTACTGCGCCCGGGAAGGATCAGGCTCCGGGTGGTA |
| | | TTTCGACTTCTGGGGAAGAGGCACCCTCGTGACTGTGTCATCT |
| | | GGGGGAGGGGGTTCCGGTGGTGGCGGATCGGGAGGAGGCGG |
| | | TTCATCCTACGTGCTGACCCAGCCACCCTCCGTGTCCGTGAGC |
| | | CCCGGCCAGACTGCATCGATTACATGTAGCGGCGACGGCCTC |
| | | TCCAAGAAATACGTGTCGTGGTACCAGCAGAAGGCCGGACAG |
| | | AGCCCGGTGGTGCTGATCTCAAGAGATAAGGAGCGGCCTAGC |
| | | GGAATCCCGGACAGGTTCTCGGGTTCCAACTCCGCGGACACT |
| | | GCTACTCTGACCATCTCGGGGACCCAGGCTATGGACGAAGCC |
| | | GATTACTACTGCCAAGCCTGGGACGACACTACTGTCGTGTTTG |
| | | GAGGGGGCACCAAGTTGACCGTCCTTACCACTACCCCAGCAC |
| | | CGAGGCCACCCACCCCGGCTCCTACCATCGCCTCCCAGCCTCT |
| | | GTCCCTGCGTCCGGAGGCATGTAGACCCGCAGCTGGTGGGGC |
| | | CGTGCATACCCGGGGTCTTGACTTCGCCTGCGATATCTACATT |
| | | TGGGCCCCTCTGGCTGGTACTTGCGGGGTCCTGCTGCTTTCAC |
| | | TCGTGATCACTCTTTACTGTAAGCGCGGTCGGAAGAAGCTGCT |
| | | GTACATCTTTAAGCAACCCTTCATGAGGCCTGTGCAGACTACT |
| | | CAAGAGGAGGACGGCTGTTCATGCCGGTTCCCAGAGGAGGAG |
| | | GAAGGCGGCTGCGAACTGCGCGTGAAATTCAGCCGCAGCGCA |
| | | GATGCTCCAGCCTACAAGCAGGGGCAGAACCAGCTCTACAAC |
| | | GAACTCAATCTTGGTCGGAGAGAGGAGTACGACGTGCTGGAC |
| | | AAGCGGAGAGGACGGGACCCAGAAATGGGCGGGAAGCCGCG |
| | | CAGAAAGAATCCCCAAGAGGGCCTGTACAACGAGCTCCAAA |
| | | AGGATAAGATGGCAGAAGCCTATAGCGAGATTGGTATGAAA |
| | | GGGGAACGCAGAAGAGGCAAAGGCCACGACGGACTGTACCA |
| | | GGGACTCAGCACCGCCACCAAGGACACCTATGACGCTCTTCA |
| | | CATGCAGGCCCTGCCGCCTCGG |

149367

| | | |
|---|---|---|
| 149367-aa | 1521 | QVQLQESGPGLVKPSQTLSLTCTVSGGSISSGGYYWSWIRQHPG |
| ScFv | | KGLEWIGYIYYSGSTYYNPSLKSRVTISVDTSKNQFSLKLSSVTA |
| domain | | ADTAVYYCARAGIAARLRGAFDIWGQGTMVTVSSGGGGSGGG |
| | | GSGGGGSDIVMTQSPSSVSASVGDRVIITCRASQGIRNWLAWYQ |
| | | QKPGKAPNLLIYAASNLQSGVPSRFSGSGSGADFTLTISSLQPEDV |
| | | ATYYCQKYNSAPFTFGPGTKVDIK |
| 149367-nt | 1522 | CAAGTGCAGCTTCAGGAGAGCGGCCCGGGACTCGTGAAGCCG |
| ScFv | | TCCCAGACCCTGTCCCTGACTTGCACCGTGTCGGGAGGAAGC |
| domain | | ATCTCGAGCGGAGGCTACTATTGGTCGTGGATTCGGCAGCAC |
| | | CCTGGAAAGGGCCTGGAATGGATCGGCTACATCTACTACTCC |
| | | GGCTCGACCTACTACAACCCATCGCTGAAGTCCAGAGTGACA |
| | | ATCTCAGTGGACACGTCCAAGAATCAGTTCAGCCTGAAGCTC |
| | | TCTTCCGTGACTGCGGCCGACACCGCCGTGTACTACTGCGCAC |
| | | GCGCTGGAATTGCCGCCCGGCTGAGGGGTGCCTTCGACATTT |
| | | GGGGACAGGGCACCATGGTCACCGTGTCCTCCGGCGGCGGAG |
| | | GTTCCGGGGGTGGAGGCTCAGGAGGAGGGGGGTCCGACATC |
| | | GTCATGACTCAGTCGCCCTCAAGCGTCAGCGCGTCCGTCGGG |
| | | GACAGAGTGATCATCACCTGTCGGGCGTCCCAGGGAATTCGC |
| | | AACTGGCTGGCCTGGTATCAGCAGAAGCCCGGAAAGGCCCCC |
| | | AACCTGTTGATCTACGCCGCCTCAAACCTCCAATCCGGGGTGC |
| | | CGAGCCGCTTCAGCGGCTCCGGTTCGGGTGCCGATTTCACTCT |
| | | GACCATCTCCTCCCTGCAACCTGAAGATGTGGCTACCTACTAC |
| | | TGCCAAAAGTACAACTCCGCACCTTTTACTTTCGGACCGGGG |
| | | ACCAAAGTGGACATTAAG |
| 149367-aa | 1523 | QVQLQESGPGLVKPSQTLSLTCTVSGGSISSGGYYWSWIRQHPG |
| VH | | KGLEWIGYIYYSGSTYYNPSLKSRVTISVDTSKNQFSLKLSSVTA |
| | | ADTAVYYCARAGIAARLRGAFDIWGQGTMVTVSS |
| 149367-aa | 1524 | DIVMTQSPSSVSASVGDRVIITCRASQGIRNWLAWYQQKPGKAP |
| VL | | NLLIYAASNLQSGVPSRFSGSGSGADFTLTISSLQPEDVATYYCQ |
| | | KYNSAPFTFGPGTKVDIK |
| 149367-aa | 1525 | MALPVTALLLPLALLLHAARPQVQLQESGPGLVKPSQTLSLTCT |
| Full CAR | | VSGGSISSGGYYWSWIRQHPGKGLEWIGYIYYSGSTYYNPSLKS |
| | | RVTISVDTSKNQFSLKLSSVTAADTAVYYCARAGIAARLRGAFDI |
| | | WGQGTMVTVSSGGGGSGGGGSGGGGSDIVMTQSPSSVSASVGD |
| | | RVIITCRASQGIRNWLAWYQQKPGKAPNLLIYAASNLQSGVPSR |
| | | FSGSGSGADFTLTISSLQPEDVATYYCQKYNSAPFTFGPGTKVDI |
| | | KTTTPAPRPPTPAPTIASQPLSLRPEACRPAAGGAVHTRGLDFAC |
| | | DIYIWAPLAGTCGVLLLSLVITLYCKRGRKKLLYIFKQPFMRPVQ |
| | | TTQEEDGCSCRFPEEEEGGCELRVKFSRSADAPAYKQGQNQLYN |
| | | ELNLGRREEYDVLDKRRGRDPEMGGKPRRKNPQEGLYNELQKD |
| | | KMAEAYSEIGMKGERRRGKGHDGLYQGLSTATKDTYDALHMQ |
| | | ALPPR |
| 149367-nt | 1526 | ATGGCCCTCCCTGTCACCGCCCTGCTGCTTCCGCTGGCTCTTC |
| Full CAR | | TGCTCCACGCCGCTCGGCCCCAAGTGCAGCTTCAGGAGAGCG |
| | | GCCCGGGACTCGTGAAGCCGTCCCAGACCCTGTCCCTGACTT |
| | | GCACCGTGTCGGGAGGAAGCATCTCGAGCGGAGGCTACTATT |
| | | GGTCGTGGATTCGGCAGCACCCTGGAAAGGGCCTGGAATGGA |
| | | TCGGCTACATCTACTACTCCGGCTCGACCTACTACAACCCATC |
| | | GCTGAAGTCCAGAGTGACAATCTCAGTGGACACGTCCAAGAA |
| | | TCAGTTCAGCCTGAAGCTCTCTTCCGTGACTGCGGCCGACACC |
| | | GCCGTGTACTACTGCGCACGCGCTGGAATTGCCGCCCGGCTG |
| | | AGGGGTGCCTTCGACATTTGGGGACAGGGCACCATGGTCACC |
| | | GTGTCCTCCGGCGGCGGAGGTTCCGGGGGTGGAGGCTCAGGA |
| | | GGAGGGGGGTCCGACATCGTCATGACTCAGTCGCCCTCAAGC |
| | | GTCAGCGCGTCCGTCGGGGACAGAGTGATCATCACCTGTCGG |
| | | GCGTCCCAGGGAATTCGCAACTGGCTGGCCTGGTATCAGCAG |
| | | AAGCCCGGAAAGGCCCCCAACCTGTTGATCTACGCCGCCTCA |
| | | AACCTCCAATCCGGGGTGCCGAGCCGCTTCAGCGGCTCCGGT |
| | | TCGGGTGCCGATTTCACTCTGACCATCTCCTCCCTGCAACCTG |
| | | AAGATGTGGCTACCTACTACTGCCAAAAGTACAACTCCGCAC |
| | | CTTTTACTTTCGGACCGGGGACCAAAGTGGACATTAAGACCA |
| | | CTACCCCAGCACCGAGGCCACCCACCCCGGCTCCTACCATCG |
| | | CCTCCCAGCCTCTGTCCCTGCGTCCGGAGGCATGTAGACCCGC |
| | | AGCTGGTGGGGCCGTGCATACCCGGGGTCTTGACTTCGCCTG |
| | | CGATATCTACATTTGGGCCCCTCTGGCTGGTACTTGCGGGGTC |
| | | CTGCTGCTTTCACTCGTGATCACTCTTTACTGTAAGCGCGGTC |
| | | GGAAGAAGCTGCTGTACATCTTTAAGCAACCCTTCATGAGGC |
| | | CTGTGCAGACTACTCAAGAGGAGGACGGCTGTTCATGCCGGT |
| | | TCCCAGAGGAGGAGGAAGGCGGCTGCGAACTGCGCGTGAAA |
| | | TTCAGCCGCAGCGCAGATGCTCCAGCCTACAAGCAGGGGCAG |
| | | AACCAGCTCTACAACGAACTCAATCTTGGTCGGAGAGAGGAG |
| | | TACGACGTGCTGGACAAGCGGAGAGGACGGGACCCAGAAAT |
| | | GGGCGGGAAGCCGCGCAGAAAGAATCCCCAAGAGGGCCTGT |
| | | ACAACGAGCTCCAAAAGGATAAGATGGCAGAAGCCTATAGC |
| | | GAGATTGGTATGAAAGGGGAACGCAGAAGAGGCAAAGGCCA |
| | | CGACGGACTGTACCAGGGACTCAGCACCGCCACCAAGGACAC |
| | | CTATGACGCTCTTCACATGCAGGCCCTGCCGCCTCGG |

149368

| | | |
|---|---|---|
| 149368-aa | 1527 | QVQLVQSGAEVKKPGSSVKVSCKASGGTFSSYAISWVRQAPGQ |
| ScFv | | GLEWMGGIIPIFGTANYAQKFQGRVTITADESTSTAYMELSSLRS |
| domain | | EDTAVYYCARRGGYQLLRWDVGLLRSAFDIWGQGTMVTVSSG |
| | | GGGSGGGGSGGGGSSYVLTQPPSVSVAPGQTARITCGGNNIGSK |
| | | SVHWYQQKPGQAPVLVLYGKNNRPSGVPDRFSGSRSGTTASLTI |
| | | TGAQAEDEADYYCSSRDSSGDHLRVFGTGTKVTVL |
| 149368-nt | 1528 | CAAGTGCAGCTGGTCCAGTCGGGCGCCGAGGTCAAGAAGCCC |
| ScFv | | GGGAGCTCTGTGAAAGTGTCCTGCAAGGCCTCCGGGGGCACC |
| domain | | TTTAGCTCCTACGCCATCTCCTGGGTCCGCCAAGCACCGGGTC |
| | | AAGGCCTGGAGTGGATGGGGGGAATTATCCCTATCTTCGGCA |
| | | CTGCCAACTACGCCCAGAAGTTCCAGGGACGCGTGACCATTA |
| | | CCGCGGACGAATCCACCTCCACCGCTTATATGGAGCTGTCCA |
| | | GCTTGCGCTCGGAAGATACCGCCGTGTACTACTGCGCCCGGA |
| | | GGGGTGGATACCAGCTGCTGAGATGGGACGTGGGCCTCCTGC |
| | | GGTCGGCGTTCGACATCTGGGGCCAGGGCACTATGGTCACTG |
| | | TGTCCAGCGGAGGAGGCGGATCGGGAGGCGGCGGATCAGGG |
| | | GGAGGCGGTTCCAGCTACGTGCTTACTCAACCCCCTTCGGTGT |
| | | CCGTGGCCCCGGGACAGACCGCCAGAATCACTTGCGGAGGAA |
| | | ACAACATTGGGTCCAAGAGCGTGCATTGGTACCAGCAGAAGC |
| | | CAGGACAGGCCCCTGTGCTGGTGCTCTACGGGAAGAACAATC |
| | | GGCCCAGCGGAGTGCCGGACAGGTTCTCGGGTTCACGCTCCG |
| | | GTACAACCGCTTCACTGACTATCACCGGGGCCCAGGCAGAGG |
| | | ATGAAGCGGACTACTACTGTTCCTCCCGGGATTCATCCGGCG |
| | | ACCACCTCCGGGTGTTCGGAACCGGAACGAAGGTCACCGTGC |
| | | TG |
| 149368-aa | 1529 | QVQLVQSGAEVKKPGSSVKVSCKASGGTFSSYAISWVRQAPGQ |
| VH | | GLEWMGGIIPIFGTANYAQKFQGRVTITADESTSTAYMELSSLRS |
| | | EDTAVYYCARRGGYQLLRWDVGLLRSAFDIWGQGTMVTVSS |
| 149368-aa | 1530 | SYVLTQPPSVSVAPGQTARITCGGNNIGSKSVHWYQQKPGQAPV |
| VL | | LVLYGKNNRPSGVPDRFSGSRSGTTASLTITGAQAEDEADYYCS |
| | | SRDSSGDHLRVFGTGTKVTVL |
| 149368-aa | 1531 | MALPVTALLLPLALLLHAARPQVQLVQSGAEVKKPGSSVKVSC |
| Full CAR | | KASGGTFSSYAISWVRQAPGQGLEWMGGIIPIFGTANYAQKFQG |
| | | RVTITADESTSTAYMELSSLRSEDTAVYYCARRGGYQLLRWDV |
| | | GLLRSAFDIWGQGTMVTVSSGGGGSGGGGSGGGGSSYVLTQPP |
| | | SVSVAPGQTARITCGGNNIGSKSVHWYQQKPGQAPVLVLYGKN |
| | | NRPSGVPDRFSGSRSGTTASLTITGAQAEDEADYYCSSRDSSGDH |
| | | LRVFGTGTKVTVLTTTPAPRPPTPAPTIASQPLSLRPEACRPAAGG |
| | | AVHTRGLDFACDIYIWAPLAGTCGVLLLSLVITLYCKRGRKKLL |
| | | YIFKQPFMRPVQTTQEEDGCSCRFPEEEEGGCELRVKFSRSADAP |
| | | AYKQGQNQLYNELNLGRREEYDVLDKRRGRDPEMGGKPRRKN |
| | | PQEGLYNELQKDKMAEAYSEIGMKGERRRGKGHDGLYQGLST |
| | | ATKDTYDALHMQALPPR |
| 149368-nt | 1532 | ATGGCCCTCCCTGTCACCGCCCTGCTGCTTCCGCTGGCTCTTC |
| Full CAR | | TGCTCCACGCCGCTCGGCCCCAAGTGCAGCTGGTCCAGTCGG |
| | | GCGCCGAGGTCAAGAAGCCCGGGAGCTCTGTGAAAGTGTCCT |
| | | GCAAGGCCTCCGGGGGCACCTTTAGCTCCTACGCCATCTCCTG |
| | | GGTCCGCCAAGCACCGGGTCAAGGCCTGGAGTGGATGGGGG |
| | | GAATTATCCCTATCTTCGGCACTGCCAACTACGCCCAGAAGTT |
| | | CCAGGGACGCGTGACCATTACCGCGGACGAATCCACCTCCAC |
| | | CGCTTATATGGAGCTGTCCAGCTTGCGCTCGGAAGATACCGC |
| | | CGTGTACTACTGCGCCCGGAGGGGTGGATACCAGCTGCTGAG |
| | | ATGGGACGTGGGCCTCCTGCGGTCGGCGTTCGACATCTGGGG |
| | | CCAGGGCACTATGGTCACTGTGTCCAGCGGAGGAGGCGGATC |
| | | GGGAGGCGGCGGATCAGGGGGAGGCGGTTCCAGCTACGTGCT |
| | | TACTCAACCCCCTTCGGTGTCCGTGGCCCCGGGACAGACCGC |
| | | CAGAATCACTTGCGGAGGAAACAACATTGGGTCCAAGAGCGT |
| | | GCATTGGTACCAGCAGAAGCCAGGACAGGCCCCTGTGCTGGT |
| | | GCTCTACGGGAAGAACAATCGGCCCAGCGGAGTGCCGGACA |
| | | GGTTCTCGGGTTCACGCTCCGGTACAACCGCTTCACTGACTAT |
| | | CACCGGGGCCCAGGCAGAGGATGAAGCGGACTACTACTGTTC |
| | | CTCCCGGGATTCATCCGGCGACCACCTCCGGGTGTTCGGAAC |
| | | CGGAACGAAGGTCACCGTGCTGACCACTACCCCAGCACCGAG |
| | | GCCACCCACCCCGGCTCCTACCATCGCCTCCCAGCCTCTGTCC |
| | | CTGCGTCCGGAGGCATGTAGACCCGCAGCTGGTGGGGCCGTG |
| | | CATACCCGGGGTCTTGACTTCGCCTGCGATATCTACATTTGGG |
| | | CCCCTCTGGCTGGTACTTGCGGGGTCCTGCTGCTTTCACTCGT |
| | | GATCACTCTTTACTGTAAGCGCGGTCGGAAGAAGCTGCTGTA |
| | | CATCTTTAAGCAACCCTTCATGAGGCCTGTGCAGACTACTCAA |
| | | GAGGAGGACGGCTGTTCATGCCGGTTCCCAGAGGAGGAGGA |
| | | AGGCGGCTGCGAACTGCGCGTGAAATTCAGCCGCAGCGCAGA |
| | | TGCTCCAGCCTACAAGCAGGGGCAGAACCAGCTCTACAACGA |
| | | ACTCAATCTTGGTCGGAGAGAGGAGTACGACGTGCTGGACAA |
| | | GCGGAGAGGACGGGACCCAGAAATGGGCGGGAAGCCGCGCA |
| | | GAAAGAATCCCCAAGAGGGCCTGTACAACGAGCTCCAAAAG |
| | | GATAAGATGGCAGAAGCCTATAGCGAGATTGGTATGAAAGG |
| | | GGAACGCAGAAGAGGCAAAGGCCACGACGGACTGTACCAGG |
| | | GACTCAGCACCGCCACCAAGGACACCTATGACGCTCTTCACA |
| | | TGCAGGCCCTGCCGCCTCGG |

149369

| | | |
|---|---|---|
| 149369-aa | 1533 | EVQLQQSGPGLVKPSQTLSLTCAISGDSVSSNSAAWNWIRQSPSR |
| ScFv | | GLEWLGRTYYRSKWYSFYAISLKSRIIINPDTSKNQFSLQLKSVTP |
| domain | | EDTAVYYCARSSPEGLFLYWFDPWGQGTLVTVSSGGDGSGGGG |
| | | SGGGGSSSELTQDPAVSVALGQTIRITCQGDSLGNYYATWYQQK |
| | | PGQAPVLVIYGTNNRPSGIPDRFSASSSGNTASLTITGAQAEDEA |
| | | DYYCNSRDSSGHHLLFGTGTKVTVL |
| 149369-nt | 1534 | GAAGTGCAGCTCCAACAGTCAGGACCGGGGCTCGTGAAGCCA |
| ScFv | | TCCCAGACCCTGTCCCTGACTTGTGCCATCTCGGGAGATAGCG |
| domain | | TGTCATCGAACTCCGCCGCCTGGAACTGGATTCGGCAGAGCC |
| | | CGTCCCGCGGACTGGAGTGGCTTGGAAGGACCTACTACCGGT |
| | | CCAAGTGGTACTCTTTCTACGCGATCTCGCTGAAGTCCCGCAT |
| | | TATCATTAACCCTGATACCTCCAAGAATCAGTTCTCCCTCCAA |
| | | CTGAAATCCGTCACCCCCGAGGACACAGCAGTGTATTACTGC |
| | | GCACGGAGCAGCCCCGAAGGACTGTTCCTGTATTGGTTTGAC |
| | | CCCTGGGGCCAGGGGACTCTTGTGACCGTGTCGAGCGGCGGA |
| | | GATGGGTCCGGTGGCGGTGGTTCGGGGGGCGGCGGATCATCA |
| | | TCCGAACTGACCCAGGACCCGGCTGTGTCCGTGGCGCTGGGA |
| | | CAAACCATCCGCATTACGTGCCAGGGAGACTCCCTGGGCAAC |
| | | TACTACGCCACTTGGTACCAGCAGAAGCCGGGCCAAGCCCCT |
| | | GTGTTGGTCATCTACGGGACCAACAACAGACCTTCCGGCATC |
| | | CCCGACCGGTTCAGCGCTTCGTCCTCCGGCAACACTGCCAGCC |
| | | TGACCATCACTGGAGCGCAGGCCGAAGATGAGGCCGACTACT |
| | | ACTGCAACAGCAGAGACTCCTCGGGTCATCACCTCTTGTTCGG |
| | | AACTGGAACCAAGGTCACCGTGCTG |
| 149369-aa | 1535 | EVQLQQSGPGLVKPSQTLSLTCAISGDSVSSNSAAWNWIRQSPSR |
| VH | | GLEWLGRTYYRSKWYSFYAISLKSRIIINPDTSKNQFSLQLKSVTP |
| | | EDTAVYYCARSSPEGLFLYWFDPWGQGTLVTVSS |
| 149369-aa | 1536 | SSELTQDPAVSVALGQTRITCQGDSLGNYYATWYQQKPGQAPV |
| VL | | LVIYGTNNRPSGIPDRFSASSSGNTASLTITGAQAEDEADYYCNS |
| | | RDSSGHHLLFGTGTKVTVL |
| 149369-aa | 1537 | MALPVTALLLPLALLLHAARPEVQLQQSGPGLVKPSQTLSLTCAI |
| Full CAR | | SGDSVSSNSAAWNWIRQSPSRGLEWLGRTYYRSKWYSFYAISLK |
| | | SRIIINPDTSKNQFSLQLKSVTPEDTAVYYCARSSPEGLFLYWFDP |
| | | WGQGTLVTVSSGGDGSGGGGSGGGGSSSELTQDPAVSVALGQT |
| | | IRITCQGDSLGNYYATWYQQKPGQAPVLVIYGTNNRPSGIPDRFS |
| | | ASSSGNTASLTITGAQAEDEADYYCNSRDSSGHHLLFGTGTKVT |
| | | VLTTTPAPRPPTPAPTIASQPLSLRPEACRPAAGGAVHTRGLDFA |
| | | CDIYIWAPLAGTCGVLLLSLVITLYCKRGRKKLLYIFKQPFMRPV |
| | | QTTQEEDGCSCRFPEEEEGGCELRVKFSRSADAPAYKQGQNQLY |
| | | NELNLGRREEYDVLDKRRGRDPEMGGKPRRKNPQEGLYNELQK |
| | | DKMAEAYSEIGMKGERRRGKGHDGLYQGLSTATKDTYDALHM |
| | | QALPPR |
| 149369-nt | 1538 | ATGGCCCTCCCTGTCACCGCCCTGCTGCTTCCGCTGGCTCTTC |
| Full CAR | | TGCTCCACGCCGCTCGGCCCGAAGTGCAGCTCCAACAGTCAG |
| | | GACCGGGGCTCGTGAAGCCATCCCAGACCCTGTCCCTGACTT |
| | | GTGCCATCTCGGGAGATAGCGTGTCATCGAACTCCGCCGCCT |
| | | GGAACTGGATTCGGCAGAGCCCGTCCCGCGGACTGGAGTGGC |
| | | TTGGAAGGACCTACTACCGGTCCAAGTGGTACTCTTTCTACGC |
| | | GATCTCGCTGAAGTCCCGCATTATCATTAACCCTGATACCTCC |
| | | AAGAATCAGTTCTCCCTCCAACTGAAATCCGTCACCCCCGAG |
| | | GACACAGCAGTGTATTACTGCGCACGGAGCAGCCCCGAAGGA |
| | | CTGTTCCTGTATTGGTTTGACCCCTGGGGCCAGGGGACTCTTG |
| | | TGACCGTGTCGAGCGGCGGAGATGGGTCCGGTGGCGGTGGTT |
| | | CGGGGGGCGGCGGATCATCATCCGAACTGACCCAGGACCCGG |
| | | CTGTGTCCGTGGCGCTGGGACAAACCATCCGCATTACGTGCC |
| | | AGGGAGACTCCCTGGGCAACTACTACGCCACTTGGTACCAGC |
| | | AGAAGCCGGGCCAAGCCCCTGTGTTGGTCATCTACGGGACCA |
| | | ACAACAGACCTTCCGGCATCCCCGACCGGTTCAGCGCTTCGTC |
| | | CTCCGGCAACACTGCCAGCCTGACCATCACTGGAGCGCAGGC |
| | | CGAAGATGAGGCCGACTACTACTGCAACAGCAGAGACTCCTC |
| | | GGGTCATCACCTCTTGTTCGGAACTGGAACCAAGGTCACCGT |
| | | GCTGACCACTACCCCAGCACCGAGGCCACCCACCCCGGCTCC |
| | | TACCATCGCCTCCCAGCCTCTGTCCCTGCGTCCGGAGGCATGT |
| | | AGACCCGCAGCTGGTGGGGCCGTGCATACCCGGGGTCTTGAC |
| | | TTCGCCTGCGATATCTACATTTGGGCCCCTCTGGCTGGTACTT |
| | | GCGGGGTCCTGCTGCTTTCACTCGTGATCACTCTTTACTGTAA |
| | | GCGCGGTCGGAAGAAGCTGCTGTACATCTTTAAGCAACCCTT |
| | | CATGAGGCCTGTGCAGACTACTCAAGAGGAGGACGGCTGTTC |
| | | ATGCCGGTTCCCAGAGGAGGAGGAAGGCGGCTGCGAACTGC |
| | | GCGTGAAATTCAGCCGCAGCGCAGATGCTCCAGCCTACAAGC |
| | | AGGGGCAGAACCAGCTCTACAACGAACTCAATCTTGGTCGGA |
| | | GAGAGGAGTACGACGTGCTGGACAAGCGGAGAGGACGGGAC |
| | | CCAGAAATGGGCGGGAAGCCGCGCAGAAAGAATCCCCAAGA |
| | | GGGCCTGTACAACGAGCTCCAAAAGGATAAGATGGCAGAAG |
| | | CCTATAGCGAGATTGGTATGAAAGGGGAACGCAGAAGAGGC |
| | | AAAGGCCACGACGGACTGTACCAGGGACTCAGCACCGCCACC |
| | | AAGGACACCTATGACGCTCTTCACATGCAGGCCCTGCCGCCT |
| | | CGG |

BCMA_EBB-C1978-A4

| | | |
|---|---|---|
| BCMA_EBB- | 1539 | EVQLVESGGGLVQPGGSLRLSCAASGFTFSSYAMSWVRQAPGK |
| C1978-A4- | | GLEWVSAISGSGGSTYYADSVKGRFTISRDNSKNTLYLQMNSLR |
| aa | | AEDTAVYYCAKVEGSGSLDYWGQGTLVTVSSGGGGSGGGGSG |
| ScFv | | GGGSEIVMTQSPGTLSLSPGERATLSCRASQSVSSAYLAWYQQK |
| domain | | PGQPPRLLISGASTRATGIPDRFGGSGSGTDFTLTISRLEPEDFAVY |
| | | YCQHYGSSFNGSSLFTFGQGTRLEIK |
| BCMA_EBB- | 1540 | GAAGTGCAGCTCGTGGAGTCAGGAGGCGGCCTGGTCCAGCCG |
| C1978-A4- | | GGAGGGTCCCTTAGACTGTCATGCGCCGCAAGCGGATTCACT |
| nt | | TTCTCCTCCTATGCCATGAGCTGGGTCCGCCAAGCCCCCGGAA |
| ScFv | | AGGGACTGGAATGGGTGTCCGCCATCTCGGGGTCTGGAGGCT |
| domain | | CAACTTACTACGCTGACTCCGTGAAGGGACGGTTCACCATTA |
| | | GCCGCGACAACTCCAAGAACACCCTCTACCTCCAAATGAACT |
| | | CCCTGCGGGCCGAGGATACCGCCGTCTACTACTGCGCCAAAG |
| | | TGGAAGGTTCAGGATCGCTGGACTACTGGGGACAGGGTACTC |
| | | TCGTGACCGTGTCATCGGGCGGAGGAGGTTCCGGCGGTGGCG |
| | | GCTCCGGCGGCGGAGGGTCGGAGATCGTGATGACCCAGAGCC |
| | | CTGGTACTCTGAGCCTTTCGCCGGGAGAAAGGGCCACCCTGT |
| | | CCTGCCGCGCTTCCCAATCCGTGTCCTCCGCGTACTTGGCGTG |
| | | GTACCAGCAGAAGCCGGGACAGCCCCCTCGGCTGCTGATCAG |
| | | CGGGGCCAGCACCCGGGCAACCGGAATCCCAGACAGATTCGG |
| | | GGGTTCCGGCAGCGGCACAGATTTCACCCTGACTATTTCGAG |
| | | GTTGGAGCCCGAGGACTTTGCGGTGTATTACTGTCAGCACTAC |
| | | GGGTCGTCCTTTAATGGCTCCAGCCTGTTCACGTTCGGACAGG |
| | | GGACCCGCCTGGAAATCAAG |
| BCMA_EBB- | 1541 | EVQLVESGGGLVQPGGSLRLSCAASGFTFSSYAMSWVRQAPGK |
| C1978-A4- | | GLEWVSAISGSGGSTYYADSVKGRFTISRDNSKNTLYLQMNSLR |
| aa | | AEDTAVYYCAKVEGSGSLDYWGQGTLVTVSS |
| VH | | |
| BCMA_EBB- | 1542 | EIVMTQSPGTLSLSPGERATLSCRASQSVSSAYLAWYQQKPGQPP |
| C1978-A4- | | RLLISGASTRATGIPDRFGGSGSGTDFTLTISRLEPEDFAVYYCQH |
| aa | | YGSSFNGSSLFTFGQGTRLEIK |
| VL | | |
| BCMA_EBB- | 1543 | MALPVTALLLPLALLLHAARPEVQLVESGGGLVQPGGSLRLSCA |
| C1978-A4- | | ASGFTFSSYAMSWVRQAPGKGLEWVSAISGSGGSTYYADSVKG |
| aa | | RFTISRDNSKNTLYLQMNSLRAEDTAVYYCAKVEGSGSLDYWG |
| Full CART | | QGTLVTVSSGGGGSGGGGSGGGGSEIVMTQSPGTLSLSPGERAT |
| | | LSCRASQSVSSAYLAWYQQKPGQPPRLLISGASTRATGIPDRFGG |
| | | SGSGTDFTLTISRLEPEDFAVYYCQHYGSSFNGSSLFTFGQGTRLE |
| | | IKTTTPAPRPPTPAPTIASQPLSLRPEACRPAAGGAVHTRGLDFAC |
| | | DIYIWAPLAGTCGVLLLSLVITLYCKRGRKKLLYIFKQPFMRPVQ |
| | | TTQEEDGCSCRFPEEEEGGCELRVKFSRSADAPAYKQGQNQLYN |
| | | ELNLGRREEYDVLDKRRGRDPEMGGKPRRKNPQEGLYNELQKD |
| | | KMAEAYSEIGMKGERRRGKGHDGLYQGLSTATKDTYDALHMQ |
| | | ALPPR |
| BCMA_EBB- | 1544 | ATGGCCCTCCCTGTCACCGCCCTGCTGCTTCCGCTGGCTCTTC |
| C1978-A4- | | TGCTCCACGCCGCTCGGCCCGAAGTGCAGCTCGTGGAGTCAG |
| nt | | GAGGCGGCCTGGTCCAGCCGGGAGGGTCCCTTAGACTGTCAT |
| Full CART | | GCGCCGCAAGCGGATTCACTTTCTCCTCCTATGCCATGAGCTG |
| | | GGTCCGCCAAGCCCCCGGAAAGGGACTGGAATGGGTGTCCGC |
| | | CATCTCGGGGTCTGGAGGCTCAACTTACTACGCTGACTCCGTG |
| | | AAGGGACGGTTCACCATTAGCCGCGACAACTCCAAGAACACC |
| | | CTCTACCTCCAAATGAACTCCCTGCGGGCCGAGGATACCGCC |
| | | GTCTACTACTGCGCCAAAGTGGAAGGTTCAGGATCGCTGGAC |
| | | TACTGGGGACAGGGTACTCTCGTGACCGTGTCATCGGGCGGA |
| | | GGAGGTTCCGGCGGTGGCGGCTCCGGCGGCGGAGGGTCGGA |
| | | GATCGTGATGACCCAGAGCCCTGGTACTCTGAGCCTTTCGCCG |
| | | GGAGAAAGGGCCACCCTGTCCTGCCGCGCTTCCCAATCCGTG |
| | | TCCTCCGCGTACTTGGCGTGGTACCAGCAGAAGCCGGGACAG |
| | | CCCCCTCGGCTGCTGATCAGCGGGGCCAGCACCCGGGCAACC |
| | | GGAATCCCAGACAGATTCGGGGGTTCCGGCAGCGGCACAGAT |
| | | TTCACCCTGACTATTTCGAGGTTGGAGCCCGAGGACTTTGCGG |
| | | TGTATTACTGTCAGCACTACGGGTCGTCCTTTAATGGCTCCAG |
| | | CCTGTTCACGTTCGGACAGGGGACCCGCCTGGAAATCAAGAC |
| | | CACTACCCCAGCACCGAGGCCACCCACCCCGGCTCCTACCAT |
| | | CGCCTCCCAGCCTCTGTCCCTGCGTCCGGAGGCATGTAGACCC |
| | | GCAGCTGGTGGGGCCGTGCATACCCGGGGTCTTGACTTCGCC |
| | | TGCGATATCTACATTTGGGCCCCTCTGGCTGGTACTTGCGGGG |
| | | TCCTGCTGCTTTCACTCGTGATCACTCTTTACTGTAAGCGCGG |
| | | TCGGAAGAAGCTGCTGTACATCTTTAAGCAACCCTTCATGAG |
| | | GCCTGTGCAGACTACTCAAGAGGAGGACGGCTGTTCATGCCG |
| | | GTTCCCAGAGGAGGAGGAAGGCGGCTGCGAACTGCGCGTGA |
| | | AATTCAGCCGCAGCGCAGATGCTCCAGCCTACAAGCAGGGGC |
| | | AGAACCAGCTCTACAACGAACTCAATCTTGGTCGGAGAGAGG |
| | | AGTACGACGTGCTGGACAAGCGGAGAGGACGGGACCCAGAA |
| | | ATGGGCGGGAAGCCGCGCAGAAAGAATCCCCAAGAGGGCCT |
| | | GTACAACGAGCTCCAAAAGGATAAGATGGCAGAAGCCTATA |
| | | GCGAGATTGGTATGAAAGGGGAACGCAGAAGAGGCAAAGGC |
| | | CACGACGGACTGTACCAGGGACTCAGCACCGCCACCAAGGAC |
| | | ACCTATGACGCTCTTCACATGCAGGCCCTGCCGCCTCGG |

BCMA_EBB-C1978-G1

| | | |
|---|---|---|
| BCMA_EBB- | 1545 | EVQLVETGGGLVQPGGSLRLSCAASGITFSRYPMSWVRQAPGKG |
| C1978-G1- | | LEWVSGISDSGVSTYYADSAKGRFTISRDNSKNTLFLQMSSLRDE |
| aa | | DTAVYYCVTRAGSEASDIWGQGTMVTVSSGGGGSGGGGSGGG |
| ScFv | | GSEIVLTQSPATLSLSPGERATLSCRASQSVSNSLAWYQQKPGQA |
| domain | | PRLLIYDASSRATGIPDRFSGSGSGTDFTLTISRLEPEDFAIYYCQQ |
| | | FGTSSGLTFGGGTKLEIK |
| BCMA_EBB- | 1546 | GAAGTGCAACTGGTGGAAACCGGTGGCGGCCTGGTGCAGCCT |
| C1978-G1- | | GGAGGATCATTGAGGCTGTCATGCGCGGCCAGCGGTATTACC |
| nt | | TTCTCCCGGTACCCCATGTCCTGGGTCAGACAGGCCCCGGGG |
| ScFv | | AAAGGGCTTGAATGGGTGTCCGGGATCTCGGACTCCGGTGTC |
| domain | | AGCACTTACTACGCCGACTCCGCCAAGGGACGCTTCACCATTT |
| | | CCCGGGACAACTCGAAGAACACCCTGTTCCTCCAAATGAGCT |
| | | CCCTCCGGGACGAGGATACTGCAGTGTACTACTGCGTGACCC |
| | | GCGCCGGGTCCGAGGCGTCTGACATTTGGGGACAGGGCACTA |
| | | TGGTCACCGTGTCGTCCGGCGGAGGGGGCTCGGGAGGCGGTG |
| | | GCAGCGGAGGAGGAGGGTCCGAGATCGTGCTGACCCAATCCC |
| | | CGGCCACCCTCTCGCTGAGCCCTGGAGAAAGGGCAACCTTGT |
| | | CCTGTCGCGCGAGCCAGTCCGTGAGCAACTCCCTGGCCTGGT |
| | | ACCAGCAGAAGCCCGGACAGGCTCCGAGACTTCTGATCTACG |
| | | ACGCTTCGAGCCGGGCCACTGGAATCCCCGACCGCTTTTCGG |
| | | GGTCCGGCTCAGGAACCGATTTCACCCTGACAATCTCACGGC |
| | | TGGAGCCAGAGGATTTCGCCATCTATTACTGCCAGCAGTTCG |
| | | GTACTTCCTCCGGCCTGACTTTCGGAGGCGGCACGAAGCTCG |
| | | AAATCAAG |
| BCMA_EBB- | 1547 | EVQLVETGGGLVQPGGSLRLSCAASGITFSRYPMSWVRQAPGKG |
| C1978-G1- | | LEWVSGISDSGVSTYYADSAKGRFTISRDNSKNTLFLQMSSLRDE |
| aa | | DTAVYYCVTRAGSEASDIWGQGTMVTVSS |
| VH | | |
| BCMA_EBB- | 1548 | EIVLTQSPATLSLSPGERATLSCRASQSVSNSLAWYQQKPGQAPR |
| C1978-G1- | | LLIYDASSRATGIPDRFSGSGSGTDFTLTISRLEPEDFAIYYCQQFG |
| aa | | TSSGLTFGGGTKLEIK |
| VL | | |
| BCMA_EBB- | 1549 | MALPVTALLLPLALLLHAARPEVQLVETGGGLVQPGGSLRLSCA |
| C1978-G1- | | ASGITFSRYPMSWVRQAPGKGLEWVSGISDSGVSTYYADSAKGR |
| aa | | FTISRDNSKNTLFLQMSSLRDEDTAVYYCVTRAGSEASDIWGQG |
| Full CART | | TMVTVSSGGGGSGGGGSGGGGSEIVLTQSPATLSLSPGERATLSC |
| | | RASQSVSNSLAWYQQKPGQAPRLLIYDASSRATGIPDRFSGSGSG |
| | | TDFTLTISRLEPEDFAIYYCQQFGTSSGLTFGGGTKLEIKTTTPAPR |
| | | PPTPAPTIASQPLSLRPEACRPAAGGAVHTRGLDFACDIYIWAPL |
| | | AGTCGVLLLSLVITLYCKRGRKKLLYIFKQPFMRPVQTTQEEDG |
| | | CSCRFPEEEEGGCELRVKFSRSADAPAYKQGQNQLYNELNLGRR |
| | | EEYDVLDKRRGRDPEMGGKPRRKNPQEGLYNELQKDKMAEAY |
| | | SEIGMKGERRRGKGHDGLYQGLSTATKDTYDALHMQALPPR |
| BCMA_EBB- | 1550 | ATGGCCCTCCCTGTCACCGCCCTGCTGCTTCCGCTGGCTCTTC |
| C1978-G1- | | TGCTCCACGCCGCTCGGCCCGAAGTGCAACTGGTGGAAACCG |
| nt | | GTGGCGGCCTGGTGCAGCCTGGAGGATCATTGAGGCTGTCAT |
| Full CART | | GCGCGGCCAGCGGTATTACCTTCTCCCGGTACCCCATGTCCTG |
| | | GGTCAGACAGGCCCCGGGGAAAGGGCTTGAATGGGTGTCCGG |
| | | GATCTCGGACTCCGGTGTCAGCACTTACTACGCCGACTCCGCC |
| | | AAGGGACGCTTCACCATTTCCCGGGACAACTCGAAGAACACC |
| | | CTGTTCCTCCAAATGAGCTCCCTCCGGGACGAGGATACTGCA |
| | | GTGTACTACTGCGTGACCCGCGCCGGGTCCGAGGCGTCTGAC |
| | | ATTTGGGGACAGGGCACTATGGTCACCGTGTCGTCCGGCGGA |
| | | GGGGGCTCGGGAGGCGGTGGCAGCGGAGGAGGAGGGTCCGA |
| | | GATCGTGCTGACCCAATCCCCGGCCACCCTCTCGCTGAGCCCT |
| | | GGAGAAAGGGCAACCTTGTCCTGTCGCGCGAGCCAGTCCGTG |
| | | AGCAACTCCCTGGCCTGGTACCAGCAGAAGCCCGGACAGGCT |
| | | CCGAGACTTCTGATCTACGACGCTTCGAGCCGGGCCACTGGA |
| | | ATCCCCGACCGCTTTTCGGGGTCCGGCTCAGGAACCGATTTCA |
| | | CCCTGACAATCTCACGGCTGGAGCCAGAGGATTTCGCCATCT |
| | | ATTACTGCCAGCAGTTCGGTACTTCCTCCGGCCTGACTTTCGG |
| | | AGGCGGCACGAAGCTCGAAATCAAGACCACTACCCCAGCACC |
| | | GAGGCCACCCACCCCGGCTCCTACCATCGCCTCCCAGCCTCTG |
| | | TCCCTGCGTCCGGAGGCATGTAGACCCGCAGCTGGTGGGGCC |
| | | GTGCATACCCGGGGTCTTGACTTCGCCTGCGATATCTACATTT |
| | | GGGCCCCTCTGGCTGGTACTTGCGGGGTCCTGCTGCTTTCACT |
| | | CGTGATCACTCTTTACTGTAAGCGCGGTCGGAAGAAGCTGCT |
| | | GTACATCTTTAAGCAACCCTTCATGAGGCCTGTGCAGACTACT |
| | | CAAGAGGAGGACGGCTGTTCATGCCGGTTCCCAGAGGAGGAG |
| | | GAAGGCGGCTGCGAACTGCGCGTGAAATTCAGCCGCAGCGCA |
| | | GATGCTCCAGCCTACAAGCAGGGGCAGAACCAGCTCTACAAC |
| | | GAACTCAATCTTGGTCGGAGAGAGGAGTACGACGTGCTGGAC |
| | | AAGCGGAGAGGACGGGACCCAGAAATGGGCGGGAAGCCGCG |
| | | CAGAAAGAATCCCCAAGAGGGCCTGTACAACGAGCTCCAAA |
| | | AGGATAAGATGGCAGAAGCCTATAGCGAGATTGGTATGAAA |
| | | GGGGAACGCAGAAGAGGCAAAGGCCACGACGGACTGTACCA |
| | | GGGACTCAGCACCGCCACCAAGGACACCTATGACGCTCTTCA |
| | | CATGCAGGCCCTGCCGCCTCGG |

BCMA_EBB-C1979-C1

| | | |
|---|---|---|
| BCMA_EBB- | 1551 | QVQLVESGGGLVQPGGSLRLSCAASGFTFSSYAMSWVRQAPGK |
| C1979-C1- | | GLEWVSAISGSGGSTYYADSVKGRFTISRDNAKNSLYLQMNSLR |
| aa | | AEDTAIYYCARATYKRELRYYYGMDVWGQGTMVTVSSGGGGS |
| ScFv | | GGGGSGGGGSEIVMTQSPGTVSLSPGERATLSCRASQSVSSSFLA |
| domain | | WYQQKPGQAPRLLIYGASSRATGIPDRFSGSGSGTDFTLTISRLEP |
| | | EDSAVYYCQQYHSSPSWTFGQGTRLEIK |
| BCMA_EBB- | 1552 | CAAGTGCAGCTCGTGGAATCGGGTGGCGGACTGGTGCAGCCG |
| C1979-C1- | | GGGGGCTCACTTAGACTGTCCTGCGCGGCCAGCGGATTCACT |
| nt | | TTCTCCTCCTACGCCATGTCCTGGGTCAGACAGGCCCCTGGAA |
| ScFv | | AGGGCCTGGAATGGGTGTCCGCAATCAGCGGCAGCGGCGGCT |
| domain | | CGACCTATTACGCGGATTCAGTGAAGGGCAGATTCACCATTT |
| | | CCCGGGACAACGCCAAGAACTCCTTGTACCTTCAAATGAACT |
| | | CCCTCCGCGCGGAAGATACCGCAATCTACTACTGCGCTCGGG |
| | | CCACTTACAAGAGGGAACTGCGCTACTACTACGGGATGGACG |
| | | TCTGGGGCCAGGGAACCATGGTCACCGTGTCCAGCGGAGGAG |
| | | GAGGATCGGGAGGAGGCGGTAGCGGGGGTGGAGGGTCGGAG |
| | | ATCGTGATGACCCAGTCCCCCGGCACTGTGTCGCTGTCCCCCG |
| | | GCGAACGGGCCACCCTGTCATGTCGGGCCAGCCAGTCAGTGT |
| | | CGTCAAGCTTCCTCGCCTGGTACCAGCAGAAACCGGGACAAG |
| | | CTCCCCGCCTGCTGATCTACGGAGCCAGCAGCCGGGCCACCG |
| | | GTATTCCTGACCGGTTCTCCGGTTCGGGGTCCGGGACCGACTT |
| | | TACTCTGACTATCTCTCGCCTCGAGCCAGAGGACTCCGCCGTG |
| | | TATTACTGCCAGCAGTACCACTCCTCCCCGTCCTGGACGTTCG |
| | | GACAGGGCACAAGGCTGGAGATTAAG |
| BCMA_EBB- | 1553 | QVQLVESGGGLVQPGGSLRLSCAASGFTFSSYAMSWVRQAPGK |
| C1979-C1- | | GLEWVSAISGSGGSTYYADSVKGRFTISRDNAKNSLYLQMNSLR |
| aa | | AEDTAIYYCARATYKRELRYYYGMDVWGQGTMVTVSS |
| VH | | |
| BCMA_EBB- | 1554 | EIVMTQSPGTVSLSPGERATLSCRASQSVSSSFLAWYQQKPGQAP |
| C1979-C1- | | RLLIYGASSRATGIPDRFSGSGSGTDFTLTISRLEPEDSAVYYCQQ |
| aa | | YHSSPSWTFGQGTRLEIK |
| VL | | |
| BCMA_EBB- | 1555 | MALPVTALLLPLALLLHAARPQVQLVESGGGLVQPGGSLRLSCA |
| C1979-C1- | | ASGFTFSSYAMSWVRQAPGKGLEWVSAISGSGGSTYYADSVKG |
| aa | | RFTISRDNAKNSLYLQMNSLRAEDTAIYYCARATYKRELRYYYG |
| Full CART | | MDVWGQGTMVTVSSGGGGSGGGGSGGGGSEIVMTQSPGTVSL |
| | | SPGERATLSCRASQSVSSSFLAWYQQKPGQAPRLLIYGASSRATG |
| | | IPDRFSGSGSGTDFTLTISRLEPEDSAVYYCQQYHSSPSWTFGQGT |
| | | RLEIKTTTPAPRPPTPAPTIASQPLSLRPEACRPAAGGAVHTRGLD |
| | | FACDIYIWAPLAGTCGVLLLSLVITLYCKRGRKKLLYIFKQPFMR |
| | | PVQTTQEEDGCSCRFPEEEEGGCELRVKFSRSADAPAYKQGQNQ |
| | | LYNELNLGRREEYDVLDKRRGRDPEMGGKPRRKNPQEGLYNEL |
| | | QKDKMAEAYSEIGMKGERRRGKGHDGLYQGLSTATKDTYDAL |
| | | HMQALPPR |
| BCMA_EBB- | 1556 | ATGGCCCTCCCTGTCACCGCCCTGCTGCTTCCGCTGGCTCTTC |
| C1979-C1- | | TGCTCCACGCCGCTCGGCCCCAAGTGCAGCTCGTGGAATCGG |
| nt | | GTGGCGGACTGGTGCAGCCGGGGGGCTCACTTAGACTGTCCT |
| Full CART | | GCGCGGCCAGCGGATTCACTTTCTCCTCCTACGCCATGTCCTG |
| | | GGTCAGACAGGCCCCTGGAAAGGGCCTGGAATGGGTGTCCGC |
| | | AATCAGCGGCAGCGGCGGCTCGACCTATTACGCGGATTCAGT |
| | | GAAGGGCAGATTCACCATTTCCCGGGACAACGCCAAGAACTC |
| | | CTTGTACCTTCAAATGAACTCCCTCCGCGCGGAAGATACCGC |
| | | AATCTACTACTGCGCTCGGGCCACTTACAAGAGGGAACTGCG |
| | | CTACTACTACGGGATGGACGTCTGGGGCCAGGGAACCATGGT |
| | | CACCGTGTCCAGCGGAGGAGGAGGATCGGGAGGAGGCGGTA |
| | | GCGGGGGTGGAGGGTCGGAGATCGTGATGACCCAGTCCCCCG |
| | | GCACTGTGTCGCTGTCCCCCGGCGAACGGGCCACCCTGTCAT |
| | | GTCGGGCCAGCCAGTCAGTGTCGTCAAGCTTCCTCGCCTGGTA |
| | | CCAGCAGAAACCGGGACAAGCTCCCCGCCTGCTGATCTACGG |
| | | AGCCAGCAGCCGGGCCACCGGTATTCCTGACCGGTTCTCCGG |
| | | TTCGGGGTCCGGGACCGACTTTACTCTGACTATCTCTCGCCTC |
| | | GAGCCAGAGGACTCCGCCGTGTATTACTGCCAGCAGTACCAC |
| | | TCCTCCCCGTCCTGGACGTTCGGACAGGGCACAAGGCTGGAG |
| | | ATTAAGACCACTACCCCAGCACCGAGGCCACCCACCCCGGCT |
| | | CCTACCATCGCCTCCCAGCCTCTGTCCCTGCGTCCGGAGGCAT |
| | | GTAGACCCGCAGCTGGTGGGGCCGTGCATACCCGGGGTCTTG |
| | | ACTTCGCCTGCGATATCTACATTTGGGCCCCTCTGGCTGGTAC |
| | | TTGCGGGGTCCTGCTGCTTTCACTCGTGATCACTCTTTACTGT |
| | | AAGCGCGGTCGGAAGAAGCTGCTGTACATCTTTAAGCAACCC |
| | | TTCATGAGGCCTGTGCAGACTACTCAAGAGGAGGACGGCTGT |
| | | TCATGCCGGTTCCCAGAGGAGGAGGAAGGCGGCTGCGAACTG |
| | | CGCGTGAAATTCAGCCGCAGCGCAGATGCTCCAGCCTACAAG |
| | | CAGGGGCAGAACCAGCTCTACAACGAACTCAATCTTGGTCGG |
| | | AGAGAGGAGTACGACGTGCTGGACAAGCGGAGAGGACGGGA |
| | | CCCAGAAATGGGCGGGAAGCCGCGCAGAAAGAATCCCCAAG |
| | | AGGGCCTGTACAACGAGCTCCAAAAGGATAAGATGGCAGAA |
| | | GCCTATAGCGAGATTGGTATGAAAGGGGAACGCAGAAGAGG |
| | | CAAAGGCCACGACGGACTGTACCAGGGACTCAGCACCGCCAC |
| | | CAAGGACACCTATGACGCTCTTCACATGCAGGCCCTGCCGCC |
| | | TCGG |

BCMA_EBB-C1978-C7

| | | |
|---|---|---|
| BCMA_EBB- | 1557 | EVQLVETGGGLVQPGGSLRLSCAASGFTFSSYAMSWVRQAPGK |
| C1978-C7- | | GLEWVSAISGSGGSTYYADSVKGRFTISRDNSKNTLYLQMNTLK |
| aa | | AEDTAVYYCARATYKRELRYYYGMDVWGQGTTVTVSSGGGGS |
| ScFv | | GGGGSGGGGSEIVLTQSPSTLSLSPGESATLSCRASQSVSTTFLA |
| domain | | WYQQKPGQAPRLLIYGSSNRATGIPDRFSGSGSGTDFTLTIRRLEP |
| | | EDFAVYYCQQYHSSPSWTFGQGTKVEIK |
| BCMA_EBB- | 1558 | GAGGTGCAGCTTGTGGAAACCGGTGGCGGACTGGTGCAGCCC |
| C1978-C7- | | GGAGGAAGCCTCAGGCTGTCCTGCGCCGCGTCCGGCTTCACC |
| nt | | TTCTCCTCGTACGCCATGTCCTGGGTCCGCCAGGCCCCCGGAA |
| ScFv | | AGGGCCTGGAATGGGTGTCCGCCATCTCTGGAAGCGGAGGTT |
| domain | | CCACGTACTACGCGGACAGCGTCAAGGGAAGGTTCACAATCT |
| | | CCCGCGATAATTCGAAGAACACTCTGTACCTTCAAATGAACA |
| | | CCCTGAAGGCCGAGGACACTGCTGTGTACTACTGCGCACGGG |
| | | CCACCTACAAGAGAGAGCTCCGGTACTACTACGGAATGGACG |
| | | TCTGGGGCCAGGGAACTACTGTGACCGTGTCCTCGGGAGGGG |
| | | GTGGCTCCGGGGGGGGCGGCTCCGGCGGAGGCGGTTCCGAGA |
| | | TTGTGCTGACCCAGTCACCTTCAACTCTGTCGCTGTCCCCGGG |
| | | AGAGAGCGCTACTCTGAGCTGCCGGGCCAGCCAGTCCGTGTC |
| | | CACCACCTTCCTCGCCTGGTATCAGCAGAAGCCGGGGCAGGC |
| | | ACCACGGCTCTTGATCTACGGGTCAAGCAACAGAGCGACCGG |
| | | AATTCCTGACCGCTTCTCGGGGAGCGGTTCAGGCACCGACTTC |
| | | ACCCTGACTATCCGGCGCCTGGAACCCGAAGATTTCGCCGTG |
| | | TATTACTGTCAACAGTACCACTCCTCGCCGTCCTGGACCTTTG |
| | | GCCAAGGAACCAAAGTGGAAATCAAG |
| BCMA_EBB- | 1559 | EVQLVETGGGLVQPGGSLRLSCAASGFTFSSYAMSWVRQAPGK |
| C1978-C7- | | GLEWVSAISGSGGSTYYADSVKGRFTISRDNSKNTLYLQMNTLK |
| aa | | AEDTAVYYCARATYKRELRYYYGMDVWGQGTTVTVSS |
| VH | | |
| BCMA_EBB- | 1560 | EIVLTQSPSTLSLSPGESATLSCRASQSVSTTFLAWYQQKPGQAP |
| C1978-C7- | | RLLIYGSSNRATGIPDRFSGSGSGTDFTLTIRRLEPEDFAVYYCQQ |
| aa | | YHSSPSWTFGQGTKVEIK |
| VL | | |
| BCMA_EBB- | 1561 | MALPVTALLLPLALLLHAARPEVQLVETGGGLVQPGGSLRLSCA |
| C1978-C7- | | ASGFTFSSYAMSWVRQAPGKGLEWVSAISGSGGSTYYADSVKG |
| aa | | RFTISRDNSKNTLYLQMNTLKAEDTAVYYCARATYKRELRYYY |
| Full CART | | GMDVWGQGTTVTVSSGGGGSGGGGSGGGGSEIVLTQSPSTLSLS |
| | | PGESATLSCRASQSVSTTFLAWYQQKPGQAPRLLIYGSSNRATGI |
| | | PDRFSGSGSGTDFTLTIRRLEPEDFAVYYCQQYHSSPSWTFGQGT |
| | | KVEIKTTTPAPRPPTPAPTIASQPLSLRPEACRPAAGGAVHTRGLD |
| | | FACDIYIWAPLAGTCGVLLLSLVITLYCKRGRKKLLYIFKQPFMR |
| | | PVQTTQEEDGCSCRFPEEEEGGCELRVKFSRSADAPAYKQGQNQ |
| | | LYNELNLGRREEYDVLDKRRGRDPEMGGKPRRKNPQEGLYNEL |
| | | QKDKMAEAYSEIGMKGERRRGKGHDGLYQGLSTATKDTYDAL |
| | | HMQALPPR |
| BCMA_EBB- | 1562 | ATGGCCCTCCCTGTCACCGCCCTGCTGCTTCCGCTGGCTCTTC |
| C1978-C7- | | TGCTCCACGCCGCTCGGCCCGAGGTGCAGCTTGTGGAAACCG |
| nt | | GTGGCGGACTGGTGCAGCCCGGAGGAAGCCTCAGGCTGTCCT |
| Full CART | | GCGCCGCGTCCGGCTTCACCTTCTCCTCGTACGCCATGTCCTG |
| | | GGTCCGCCAGGCCCCCGGAAAGGGCCTGGAATGGGTGTCCGC |
| | | CATCTCTGGAAGCGGAGGTTCCACGTACTACGCGGACAGCGT |
| | | CAAGGGAAGGTTCACAATCTCCCGCGATAATTCGAAGAACAC |
| | | TCTGTACCTTCAAATGAACACCCTGAAGGCCGAGGACACTGC |
| | | TGTGTACTACTGCGCACGGGCCACCTACAAGAGAGAGCTCCG |
| | | GTACTACTACGGAATGGACGTCTGGGGCCAGGGAACTACTGT |
| | | GACCGTGTCCTCGGGAGGGGGTGGCTCCGGGGGGGGCGGCTC |
| | | CGGCGGAGGCGGTTCCGAGATTGTGCTGACCCAGTCACCTTC |
| | | AACTCTGTCGCTGTCCCCGGGAGAGAGCGCTACTCTGAGCTG |
| | | CCGGGCCAGCCAGTCCGTGTCCACCACCTTCCTCGCCTGGTAT |
| | | CAGCAGAAGCCGGGGCAGGCACCACGGCTCTTGATCTACGGG |
| | | TCAAGCAACAGAGCGACCGGAATTCCTGACCGCTTCTCGGGG |
| | | AGCGGTTCAGGCACCGACTTCACCCTGACTATCCGGCGCCTG |
| | | GAACCCGAAGATTTCGCCGTGTATTACTGTCAACAGTACCACT |
| | | CCTCGCCGTCCTGGACCTTTGGCCAAGGAACCAAAGTGGAAA |
| | | TCAAGACCACTACCCCAGCACCGAGGCCACCCACCCCGGCTC |
| | | CTACCATCGCCTCCCAGCCTCTGTCCCTGCGTCCGGAGGCATG |
| | | TAGACCCGCAGCTGGTGGGGCCGTGCATACCCGGGGTCTTGA |
| | | CTTCGCCTGCGATATCTACATTTGGGCCCCTCTGGCTGGTACT |
| | | TGCGGGGTCCTGCTGCTTTCACTCGTGATCACTCTTTACTGTA |
| | | AGCGCGGTCGGAAGAAGCTGCTGTACATCTTTAAGCAACCCT |
| | | TCATGAGGCCTGTGCAGACTACTCAAGAGGAGGACGGCTGTT |
| | | CATGCCGGTTCCCAGAGGAGGAGGAAGGCGGCTGCGAACTGC |
| | | GCGTGAAATTCAGCCGCAGCGCAGATGCTCCAGCCTACAAGC |
| | | AGGGGCAGAACCAGCTCTACAACGAACTCAATCTTGGTCGGA |
| | | GAGAGGAGTACGACGTGCTGGACAAGCGGAGAGGACGGGAC |
| | | CCAGAAATGGGCGGGAAGCCGCGCAGAAAGAATCCCCAAGA |
| | | GGGCCTGTACAACGAGCTCCAAAAGGATAAGATGGCAGAAG |
| | | CCTATAGCGAGATTGGTATGAAAGGGGAACGCAGAAGAGGC |
| | | AAAGGCCACGACGGACTGTACCAGGGACTCAGCACCGCCACC |
| | | AAGGACACCTATGACGCTCTTCACATGCAGGCCCTGCCGCCT |
| | | CGG |

BCMA_EBB-C1978-D10

| | | |
|---|---|---|
| BCMA_EBB- | 1563 | EVQLVETGGGLVQPGRSLRLSCAASGFTFDDYAMHWVRQAPGK |
| C1978- | | GLEWVSGISWNSGSIGYADSVKGRFTISRDNAKNSLYLQMNSLR |
| D10-aa | | DEDTAVYYCARVGKAVPDVWGQGTTVTVSSGGGGSGGGGSGG |
| ScFv | | GGSDIVMTQTPSSLSASVGDRVTITCRASQSISSYLNWYQQKPGK |
| domain | | APKLLIYAASSLQSGVPSRFSGSGSGTDFTLTISSLQPEDFATYYC |
| | | QQSYSTPYSFGQGTRLEIK |
| BCMA_EBB- | 1564 | GAAGTGCAGCTCGTGGAAACTGGAGGTGGACTCGTGCAGCCT |
| C1978- | | GGACGGTCGCTGCGGCTGAGCTGCGCTGCATCCGGCTTCACC |
| D10-nt | | TTCGACGATTATGCCATGCACTGGGTCAGACAGGCGCCAGGG |
| ScFv | | AAGGGACTTGAGTGGGTGTCCGGTATCAGCTGGAATAGCGGC |
| domain | | TCAATCGGATACGCGGACTCCGTGAAGGGAAGGTTCACCATT |
| | | TCCCGCGACAACGCCAAGAACTCCCTGTACTTGCAAATGAAC |
| | | AGCCTCCGGGATGAGGACACTGCCGTGTACTACTGCGCCCGC |
| | | GTCGGAAAAGCTGTGCCCGACGTCTGGGGCCAGGGAACCACT |
| | | GTGACCGTGTCCAGCGGCGGGGGTGGATCGGGCGGTGGAGG |
| | | GTCCGGTGGAGGGGGCTCAGATATTGTGATGACCCAGACCCC |
| | | CTCGTCCCTGTCCGCCTCGGTCGGCGACCGCGTGACTATCACA |
| | | TGTAGAGCCTCGCAGAGCATCTCCAGCTACCTGAACTGGTAT |
| | | CAGCAGAAGCCGGGGAAGGCCCCGAAGCTCCTGATCTACGCG |
| | | GCATCATCACTGCAATCGGGAGTGCCGAGCCGGTTTTCCGGG |
| | | TCCGGCTCCGGCACCGACTTCACGCTGACCATTTCTTCCCTGC |
| | | AACCCGAGGACTTCGCCACTTACTACTGCCAGCAGTCCTACTC |
| | | CACCCCTTACTCCTTCGGCCAAGGAACCAGGCTGGAAATCAAG |
| BCMA_EBB- | 1565 | EVQLVETGGGLVQPGRSLRLSCAASGFTFDDYAMHWVRQAPGK |
| C1978- | | GLEWVSGISWNSGSIGYADSVKGRFTISRDNAKNSLYLQMNSLR |
| D10-aa | | DEDTAVYYCARVGKAVPDVWGQGTTVTVSS |
| VH | | |
| BCMA_EBB- | 1566 | DIVMTQTPSSLSASVGDRVTITCRASQSISSYLNWYQQKPGKAPK |
| C1978- | | LLIYAASSLQSGVPSRFSGSGSGTDFTLTISSLQPEDFATYYCQQS |
| D10-aa | | YSTPYSFGQGTRLEIK |
| VL | | |
| BCMA_EBB- | 1567 | MALPVTALLLPLALLLHAARPEVQLVETGGGLVQPGRSLRLSCA |
| C1978- | | ASGFTFDDYAMHWVRQAPGKGLEWVSGISWNSGSIGYADSVK |
| D10-aa | | GRFTISRDNAKNSLYLQMNSLRDEDTAVYYCARVGKAVPDVW |
| Full CART | | GQGTTVTVSSGGGGSGGGGSGGGGSDIVMTQTPSSLSASVGDRV |
| | | TITCRASQSISSYLNWYQQKPGKAPKLLIYAASSLQSGVPSRFSGS |
| | | GSGTDFTLTISSLQPEDFATYYCQQSYSTPYSFGQGTRLEIKTTTP |
| | | APRPPTPAPTIASQPLSLRPEACRPAAGGAVHTRGLDFACDIYIW |
| | | APLAGTCGVLLLSLVITLYCKRGRKKLLYIFKQPFMRPVQTTQEE |
| | | DGCSCRFPEEEEGGCELRVKFSRSADAPAYKQGQNQLYNELNLG |
| | | RREEYDVLDKRRGRDPEMGGKPRRKNPQEGLYNELQKDKMAE |
| | | AYSEIGMKGERRRGKGHDGLYQGLSTATKDTYDALHMQALPPR |
| BCMA_EBB- | 1568 | ATGGCCCTCCCTGTCACCGCCCTGCTGCTTCCGCTGGCTCTTC |
| C1978- | | TGCTCCACGCCGCTCGGCCCGAAGTGCAGCTCGTGGAAACTG |
| D10-nt | | GAGGTGGACTCGTGCAGCCTGGACGGTCGCTGCGGCTGAGCT |
| Full CART | | GCGCTGCATCCGGCTTCACCTTCGACGATTATGCCATGCACTG |
| | | GGTCAGACAGGCGCCAGGGAAGGGACTTGAGTGGGTGTCCG |
| | | GTATCAGCTGGAATAGCGGCTCAATCGGATACGCGGACTCCG |
| | | TGAAGGGAAGGTTCACCATTTCCCGCGACAACGCCAAGAACT |
| | | CCCTGTACTTGCAAATGAACAGCCTCCGGGATGAGGACACTG |
| | | CCGTGTACTACTGCGCCCGCGTCGGAAAAGCTGTGCCCGACG |
| | | TCTGGGGCCAGGGAACCACTGTGACCGTGTCCAGCGGCGGGG |
| | | GTGGATCGGGCGGTGGAGGGTCCGGTGGAGGGGGCTCAGAT |
| | | ATTGTGATGACCCAGACCCCCTCGTCCCTGTCCGCCTCGGTCG |
| | | GCGACCGCGTGACTATCACATGTAGAGCCTCGCAGAGCATCT |
| | | CCAGCTACCTGAACTGGTATCAGCAGAAGCCGGGGAAGGCCC |
| | | CGAAGCTCCTGATCTACGCGGCATCATCACTGCAATCGGGAG |
| | | TGCCGAGCCGGTTTTCCGGGTCCGGCTCCGGCACCGACTTCAC |
| | | GCTGACCATTTCTTCCCTGCAACCCGAGGACTTCGCCACTTAC |
| | | TACTGCCAGCAGTCCTACTCCACCCCTTACTCCTTCGGCCAAG |
| | | GAACCAGGCTGGAAATCAAGACCACTACCCCAGCACCGAGGC |
| | | CACCCACCCCGGCTCCTACCATCGCCTCCCAGCCTCTGTCCCT |
| | | GCGTCCGGAGGCATGTAGACCCGCAGCTGGTGGGGCCGTGCA |
| | | TACCCGGGGTCTTGACTTCGCCTGCGATATCTACATTTGGGCC |
| | | CCTCTGGCTGGTACTTGCGGGGTCCTGCTGCTTTCACTCGTGA |
| | | TCACTCTTTACTGTAAGCGCGGTCGGAAGAAGCTGCTGTACAT |
| | | CTTTAAGCAACCCTTCATGAGGCCTGTGCAGACTACTCAAGA |
| | | GGAGGACGGCTGTTCATGCCGGTTCCCAGAGGAGGAGGAAG |
| | | GCGGCTGCGAACTGCGCGTGAAATTCAGCCGCAGCGCAGATG |
| | | CTCCAGCCTACAAGCAGGGGCAGAACCAGCTCTACAACGAAC |
| | | TCAATCTTGGTCGGAGAGAGGAGTACGACGTGCTGGACAAGC |
| | | GGAGAGGACGGGACCCAGAAATGGGCGGGAAGCCGCGCAGA |
| | | AAGAATCCCCAAGAGGGCCTGTACAACGAGCTCCAAAAGGAT |
| | | AAGATGGCAGAAGCCTATAGCGAGATTGGTATGAAAGGGGA |
| | | ACGCAGAAGAGGCAAAGGCCACGACGGACTGTACCAGGGAC |
| | | TCAGCACCGCCACCAAGGACACCTATGACGCTCTTCACATGC |
| | | AGGCCCTGCCGCCTCGG |

BCMA_EBB-C1979-C12

| | | |
|---|---|---|
| BCMA_EBB- | 1569 | EVQLVESGGGLVQPGRSLRLSCTASGFTFDDYAMHWVRQRPGK |
| C1979- | | GLEWVASINWKGNSLAYGDSVKGRFAISRDNAKNTVFLQMNSL |
| C12-aa | | RTEDTAVYYCASHQGVAYYNYAMDVWGRGTLVTVSSGGGGS |
| ScFv | | GGGGSGGGGSEIVLTQSPGTLSLSPGERATLSCRATQSIGSSFLA |
| domain | | WYQQRPGQAPRLLIYGASQRATGIPDRFSGRGSGTDFTLTISRVE |
| | | PEDSAVYYCQHYESSPSWTFGQGTKVEIK |
| BCMA_EBB- | 1570 | GAAGTGCAGCTCGTGGAGAGCGGGGGAGGATTGGTGCAGCC |
| C1979- | | CGGAAGGTCCCTGCGGCTCTCCTGCACTGCGTCTGGCTTCACC |
| C12-nt | | TTCGACGACTACGCGATGCACTGGGTCAGACAGCGCCCGGGA |
| ScFv | | AAGGGCCTGGAATGGGTCGCCTCAATCAACTGGAAGGGAAAC |
| domain | | TCCCTGGCCTATGGCGACAGCGTGAAGGGCCGCTTCGCCATTT |
| | | CGCGCGACAACGCCAAGAACACCGTGTTTCTGCAAATGAATT |
| | | CCCTGCGGACCGAGGATACCGCTGTGTACTACTGCGCCAGCC |
| | | ACCAGGGCGTGGCATACTATAACTACGCCATGGACGTGTGGG |
| | | GAAGAGGGACGCTCGTCACCGTGTCCTCCGGGGGCGGTGGAT |
| | | CGGGTGGAGGAGGAAGCGGTGGCGGGGGCAGCGAAATCGTG |
| | | CTGACTCAGAGCCCGGGAACTCTTTCACTGTCCCCGGGAGAA |
| | | CGGGCCACTCTCTCGTGCCGGGCCACCCAGTCCATCGGCTCCT |
| | | CCTTCCTTGCCTGGTACCAGCAGAGGCCAGGACAGGCGCCCC |
| | | GCCTGCTGATCTACGGTGCTTCCCAACGCGCCACTGGCATTCC |
| | | TGACCGGTTCAGCGGCAGAGGGTCGGGAACCGATTTCACACT |
| | | GACCATTTCCCGGGTGGAGCCCGAAGATTCGGCAGTCTACTA |
| | | CTGTCAGCATTACGAGTCCTCCCCTTCATGGACCTTCGGTCAA |
| | | GGGACCAAAGTGGAGATCAAG |
| BCMA_EBB- | 1571 | EVQLVESGGGLVQPGRSLRLSCTASGFTFDDYAMHWVRQRPGK |
| C1979- | | GLEWVASINWKGNSLAYGDSVKGRFAISRDNAKNTVFLQMNSL |
| C12-aa | | RTEDTAVYYCASHQGVAYYNYAMDVWGRGTLVTVSS |
| VH | | |
| BCMA_EBB- | 1572 | EIVLTQSPGTLSLSPGERATLSCRATQSIGSSFLAWYQQRPGQAPR |
| C1979- | | LLIYGASQRATGIPDRFSGRGSGTDFTLTISRVEPEDSAVYYCQH |
| C12-aa | | YESSPSWTFGQGTKVEIK |
| VL | | |
| BCMA_EBB- | 1573 | MALPVTALLLPLALLLHAARPEVQLVESGGGLVQPGRSLRLSCT |
| C1979- | | ASGFTFDDYAMHWVRQRPGKGLEWVASINWKGNSLAYGDSVK |
| C12-aa | | GRFAISRDNAKNTVFLQMNSLRTEDTAVYYCASHQGVAYYNYA |
| Full CART | | MDVWGRGTLVTVSSGGGGSGGGGSGGGGSEIVLTQSPGTLSLSP |
| | | GERATLSCRATQSIGSSFLAWYQQRPGQAPRLLIYGASQRATGIP |
| | | DRFSGRGSGTDFTLTISRVEPEDSAVYYCQHYESSPSWTFGQGTK |
| | | VEIKTTTPAPRPPTPAPTIASQPLSLRPEACRPAAGGAVHTRGLDF |
| | | ACDIYIWAPLAGTCGVLLLSLVITLYCKRGRKKLLYIFKQPFMRP |
| | | VQTTQEEDGCSCRFPEEEEGGCELRVKFSRSADAPAYKQGQNQL |
| | | YNELNLGRREEYDVLDKRRGRDPEMGGKPRRKNPQEGLYNELQ |
| | | KDKMAEAYSEIGMKGERRRGKGHDGLYQGLSTATKDTYDALH |
| | | MQALPPR |
| BCMA_EBB- | 1574 | ATGGCCCTCCCTGTCACCGCCCTGCTGCTTCCGCTGGCTCTTC |
| C1979- | | TGCTCCACGCCGCTCGGCCCGAAGTGCAGCTCGTGGAGAGCG |
| C12-nt | | GGGGAGGATTGGTGCAGCCCGGAAGGTCCCTGCGGCTCTCCT |
| Full CART | | GCACTGCGTCTGGCTTCACCTTCGACGACTACGCGATGCACTG |
| | | GGTCAGACAGCGCCCGGGAAAGGGCCTGGAATGGGTCGCCTC |
| | | AATCAACTGGAAGGGAAACTCCCTGGCCTATGGCGACAGCGT |
| | | GAAGGGCCGCTTCGCCATTTCGCGCGACAACGCCAAGAACAC |
| | | CGTGTTTCTGCAAATGAATTCCCTGCGGACCGAGGATACCGCT |
| | | GTGTACTACTGCGCCAGCCACCAGGGCGTGGCATACTATAAC |
| | | TACGCCATGGACGTGTGGGGAAGAGGGACGCTCGTCACCGTG |
| | | TCCTCCGGGGGCGGTGGATCGGGTGGAGGAGGAAGCGGTGG |
| | | CGGGGGCAGCGAAATCGTGCTGACTCAGAGCCCGGGAACTCT |
| | | TTCACTGTCCCCGGGAGAACGGGCCACTCTCTCGTGCCGGGC |
| | | CACCCAGTCCATCGGCTCCTCCTTCCTTGCCTGGTACCAGCAG |
| | | AGGCCAGGACAGGCGCCCCGCCTGCTGATCTACGGTGCTTCC |
| | | CAACGCGCCACTGGCATTCCTGACCGGTTCAGCGGCAGAGGG |
| | | TCGGGAACCGATTTCACACTGACCATTTCCCGGGTGGAGCCC |
| | | GAAGATTCGGCAGTCTACTACTGTCAGCATTACGAGTCCTCCC |
| | | CTTCATGGACCTTCGGTCAAGGGACCAAAGTGGAGATCAAGA |
| | | CCACTACCCCAGCACCGAGGCCACCCACCCCGGCTCCTACCA |
| | | TCGCCTCCCAGCCTCTGTCCCTGCGTCCGGAGGCATGTAGACC |
| | | CGCAGCTGGTGGGGCCGTGCATACCCGGGGTCTTGACTTCGC |
| | | CTGCGATATCTACATTTGGGCCCCTCTGGCTGGTACTTGCGGG |
| | | GTCCTGCTGCTTTCACTCGTGATCACTCTTTACTGTAAGCGCG |
| | | GTCGGAAGAAGCTGCTGTACATCTTTAAGCAACCCTTCATGA |
| | | GGCCTGTGCAGACTACTCAAGAGGAGGACGGCTGTTCATGCC |
| | | GGTTCCCAGAGGAGGAGGAAGGCGGCTGCGAACTGCGCGTG |
| | | AAATTCAGCCGCAGCGCAGATGCTCCAGCCTACAAGCAGGGG |
| | | CAGAACCAGCTCTACAACGAACTCAATCTTGGTCGGAGAGAG |
| | | GAGTACGACGTGCTGGACAAGCGGAGAGGACGGGACCCAGA |
| | | AATGGGCGGGAAGCCGCGCAGAAAGAATCCCCAAGAGGGCC |
| | | TGTACAACGAGCTCCAAAAGGATAAGATGGCAGAAGCCTATA |
| | | GCGAGATTGGTATGAAAGGGGAACGCAGAAGAGGCAAAGGC |
| | | CACGACGGACTGTACCAGGGACTCAGCACCGCCACCAAGGAC |
| | | ACCTATGACGCTCTTCACATGCAGGCCCTGCCGCCTCGG |

BCMA_EBB-C1980-G4

| | | |
|---|---|---|
| BCMA_EBB- | 1575 | EVQLVESGGGLVQPGGSLRLSCAASGFTFSSYAMSWVRQAPGK |
| C1980- | | GLEWVSAISGSGGSTYYADSVKGRFTISRDNSKNTLYLQMNSLR |
| G4-aa | | AEDTAVYYCAKVVRDGMDVWGQGTTVTVSSGGGGSGGGGSG |
| ScFv | | GGGSEIVLTQSPATLSLSPGERATLSCRASQSVSSSYLAWYQQKP |
| domain | | GQAPRLLIYGASSRATGIPDRFSGNGSGTDFTLTISRLEPEDFAVY |
| | | YCQQYGSPPRFTFGPGTKVDIK |
| BCMA_EBB- | 1576 | GAGGTGCAGTTGGTCGAAAGCGGGGGCGGGCTTGTGCAGCCT |
| C1980- | | GGCGGATCACTGCGGCTGTCCTGCGCGGCATCAGGCTTCACG |
| G4-nt | | TTTTCTTCCTACGCCATGTCCTGGGTGCGCCAGGCCCCTGGAA |
| ScFv | | AGGGACTGGAATGGGTGTCCGCGATTTCGGGGTCCGGCGGGA |
| domain | | GCACCTACTACGCCGATTCCGTGAAGGGCCGCTTCACTATCTC |
| | | GCGGGACAACTCCAAGAACACCCTCTACCTCCAAATGAATAG |
| | | CCTGCGGGCCGAGGATACCGCCGTCTACTATTGCGCTAAGGT |
| | | CGTGCGCGACGGAATGGACGTGTGGGGACAGGGTACCACCGT |
| | | GACAGTGTCCTCGGGGGGAGGCGGTAGCGGCGGAGGAGGAA |
| | | GCGGTGGTGGAGGTTCCGAGATTGTGCTGACTCAATCACCCG |
| | | CGACCCTGAGCCTGTCCCCCGGCGAAAGGGCCACTCTGTCCT |
| | | GTCGGGCCAGCCAATCAGTCTCCTCCTCGTACCTGGCCTGGTA |
| | | CCAGCAGAAGCCAGGACAGGCTCCGAGACTCCTTATCTATGG |
| | | CGCATCCTCCCGCGCCACCGGAATCCCGGATAGGTTCTCGGG |
| | | AAACGGATCGGGGACCGACTTCACTCTCACCATCTCCCGGCT |
| | | GGAACCGGAGGACTTCGCCGTGTACTACTGCCAGCAGTACGG |
| | | CAGCCCGCCTAGATTCACTTTCGGCCCCGGCACCAAAGTGGA |
| | | CATCAAG |
| BCMA_EBB- | 1577 | EVQLVESGGGLVQPGGSLRLSCAASGFTFSSYAMSWVRQAPGK |
| C1980- | | GLEWVSAISGSGGSTYYADSVKGRFTISRDNSKNTLYLQMNSLR |
| G4-aa | | AEDTAVYYCAKVVRDGMDVWGQGTTVTVSS |
| VH | | |
| BCMA_EBB- | 1578 | EIVLTQSPATLSLSPGERATLSCRASQSVSSSYLAWYQQKPGQAP |
| C1980- | | RLLIYGASSRATGIPDRFSGNGSGTDFTLTISRLEPEDFAVYYCQQ |
| G4-aa | | YGSPPRFTFGPGTKVDIK |
| VL | | |
| BCMA_EBB- | 1579 | MALPVTALLLPLALLLHAARPEVQLVESGGGLVQPGGSLRLSCA |
| C1980- | | ASGFTFSSYAMSWVRQAPGKGLEWVSAISGSGGSTYYADSVKG |
| G4-aa | | RFTISRDNSKNTLYLQMNSLRAEDTAVYYCAKVVRDGMDVWG |
| Full CART | | QGTTVTVSSGGGGSGGGGSGGGGSEIVLTQSPATLSLSPGERATL |
| | | SCRASQSVSSSYLAWYQQKPGQAPRLLIYGASSRATGIPDRFSGN |
| | | GSGTDFTLTISRLEPEDFAVYYCQQYGSPPRFTFGPGTKVDIKTTT |
| | | PAPRPPTPAPTIASQPLSLRPEACRPAAGGAVHTRGLDFACDIYIW |
| | | APLAGTCGVLLLSLVITLYCKRGRKKLLYIFKQPFMRPVQTTQEE |
| | | DGCSCRFPEEEEGGCELRVKFSRSADAPAYKQGQNQLYNELNLG |
| | | RREEYDVLDKRRGRDPEMGGKPRRKNPQEGLYNELQKDKMAE |
| | | AYSEIGMKGERRRGKGHDGLYQGLSTATKDTYDALHMQALPPR |
| BCMA_EBB- | 1580 | ATGGCCCTCCCTGTCACCGCCCTGCTGCTTCCGCTGGCTCTTC |
| C1980- | | TGCTCCACGCCGCTCGGCCCGAGGTGCAGTTGGTCGAAAGCG |
| G4-nt | | GGGGCGGGCTTGTGCAGCCTGGCGGATCACTGCGGCTGTCCT |
| Full CART | | GCGCGGCATCAGGCTTCACGTTTTCTTCCTACGCCATGTCCTG |
| | | GGTGCGCCAGGCCCCTGGAAAGGGACTGGAATGGGTGTCCGC |
| | | GATTTCGGGGTCCGGCGGGAGCACCTACTACGCCGATTCCGT |
| | | GAAGGGCCGCTTCACTATCTCGCGGGACAACTCCAAGAACAC |
| | | CCTCTACCTCCAAATGAATAGCCTGCGGGCCGAGGATACCGC |
| | | CGTCTACTATTGCGCTAAGGTCGTGCGCGACGGAATGGACGT |
| | | GTGGGGACAGGGTACCACCGTGACAGTGTCCTCGGGGGGAGG |
| | | CGGTAGCGGCGGAGGAGGAAGCGGTGGTGGAGGTTCCGAGA |
| | | TTGTGCTGACTCAATCACCCGCGACCCTGAGCCTGTCCCCCGG |
| | | CGAAAGGGCCACTCTGTCCTGTCGGGCCAGCCAATCAGTCTC |
| | | CTCCTCGTACCTGGCCTGGTACCAGCAGAAGCCAGGACAGGC |
| | | TCCGAGACTCCTTATCTATGGCGCATCCTCCCGCGCCACCGGA |
| | | ATCCCGGATAGGTTCTCGGGAAACGGATCGGGGACCGACTTC |
| | | ACTCTCACCATCTCCCGGCTGGAACCGGAGGACTTCGCCGTGT |
| | | ACTACTGCCAGCAGTACGGCAGCCCGCCTAGATTCACTTTCG |
| | | GCCCCGGCACCAAAGTGGACATCAAGACCACTACCCCAGCAC |
| | | CGAGGCCACCCACCCCGGCTCCTACCATCGCCTCCCAGCCTCT |
| | | GTCCCTGCGTCCGGAGGCATGTAGACCCGCAGCTGGTGGGGC |
| | | CGTGCATACCCGGGGTCTTGACTTCGCCTGCGATATCTACATT |
| | | TGGGCCCCTCTGGCTGGTACTTGCGGGGTCCTGCTGCTTTCAC |
| | | TCGTGATCACTCTTTACTGTAAGCGCGGTCGGAAGAAGCTGCT |
| | | GTACATCTTTAAGCAACCCTTCATGAGGCCTGTGCAGACTACT |
| | | CAAGAGGAGGACGGCTGTTCATGCCGGTTCCCAGAGGAGGAG |
| | | GAAGGCGGCTGCGAACTGCGCGTGAAATTCAGCCGCAGCGCA |
| | | GATGCTCCAGCCTACAAGCAGGGGCAGAACCAGCTCTACAAC |
| | | GAACTCAATCTTGGTCGGAGAGAGGAGTACGACGTGCTGGAC |
| | | AAGCGGAGAGGACGGGACCCAGAAATGGGCGGGAAGCCGCG |
| | | CAGAAAGAATCCCCAAGAGGGCCTGTACAACGAGCTCCAAA |
| | | AGGATAAGATGGCAGAAGCCTATAGCGAGATTGGTATGAAA |
| | | GGGGAACGCAGAAGAGGCAAAGGCCACGACGGACTGTACCA |
| | | GGGACTCAGCACCGCCACCAAGGACACCTATGACGCTCTTCA |
| | | CATGCAGGCCCTGCCGCCTCGG |

BCMA_EBB-C1980-D2

| | | |
|---|---|---|
| BCMA_EBB- | 1581 | EVQLLESGGGLVQPGGSLRLSCAASGFTFSSYAMSWVRQAPGK |
| C1980- | | GLEWVSAISGSGGSTYYADSVKGRFTISRDNSKNTLYLQMNSLR |
| D2-aa | | AEDTAVYYCAKIPQTGTFDYWGQGTLVTVSSGGGGSGGGGSGG |
| ScFv | | GGSEIVLTQSPGTLSLSPGERATLSCRASQSVSSSYLAWYQQRPG |
| domain | | QAPRLLIYGASSRATGIPDRFSGSGSGTDFTLTISRLEPEDFAVYY |
| | | CQHYGSSPSWTFGQGTRLEIK |
| BCMA_EBB- | 1582 | GAAGTGCAGCTGCTGGAGTCCGGCGGTGGATTGGTGCAACCG |
| C1980- | | GGGGGATCGCTCAGACTGTCCTGTGCGGCGTCAGGCTTCACC |
| D2-nt | | TTCTCGAGCTACGCCATGTCATGGGTCAGACAGGCCCCTGGA |
| ScFv | | AAGGGTCTGGAATGGGTGTCCGCCATTTCCGGGAGCGGGGGA |
| domain | | TCTACATACTACGCCGATAGCGTGAAGGGCCGCTTCACCATTT |
| | | CCCGGGACAACTCCAAGAACACTCTCTATCTGCAAATGAACT |
| | | CCCTCCGCGCTGAGGACACTGCCGTGTACTACTGCGCCAAAA |
| | | TCCCTCAGACCGGCACCTTCGACTACTGGGGACAGGGGACTC |
| | | TGGTCACCGTCAGCAGCGGTGGCGGAGGTTCGGGGGGAGGA |
| | | GGAAGCGGCGGCGGAGGGTCCGAGATTGTGCTGACCCAGTCA |
| | | CCCGGCACTTTGTCCCTGTCGCCTGGAGAAAGGGCCACCCTTT |
| | | CCTGCCGGGCATCCCAATCCGTGTCCTCCTCGTACCTGGCCTG |
| | | GTACCAGCAGAGGCCCGGACAGGCCCCACGGCTTCTGATCTA |
| | | CGGAGCAAGCAGCCGCGCGACCGGTATCCCGGACCGGTTTTC |
| | | GGGCTCGGGCTCAGGAACTGACTTCACCCTCACCATCTCCCGC |
| | | CTGGAACCCGAAGATTTCGCTGTGTATTACTGCCAGCACTACG |
| | | GCAGCTCCCCGTCCTGGACGTTCGGCCAGGGAACTCGGCTGG |
| | | AGATCAAG |
| BCMA_EBB- | 1583 | EVQLLESGGGLVQPGGSLRLSCAASGFTFSSYAMSWVRQAPGK |
| C1980- | | GLEWVSAISGSGGSTYYADSVKGRFTISRDNSKNTLYLQMNSLR |
| D2-aa | | AEDTAVYYCAKIPQTGTFDYWGQGTLVTVSS |
| VH | | |
| BCMA_EBB- | 1584 | EIVLTQSPGTLSLSPGERATLSCRASQSVSSSYLAWYQQRPGQAP |
| C1980- | | RLLIYGASSRATGIPDRFSGSGSGTDFTLTISRLEPEDFAVYYCQH |
| D2-aa | | YGSSPSWTFGQGTRLEIK |
| VL | | |
| BCMA_EBB- | 1585 | MALPVTALLLPLALLLHAARPEVQLLESGGGLVQPGGSLRLSCA |
| C1980- | | ASGFTFSSYAMSWVRQAPGKGLEWVSAISGSGGSTYYADSVKG |
| D2-aa | | RFTISRDNSKNTLYLQMNSLRAEDTAVYYCAKIPQTGTFDYWGQ |
| Full CART | | GTLVTVSSGGGGSGGGGSGGGGSEIVLTQSPGTLSLSPGERATLS |
| | | CRASQSVSSSYLAWYQQRPGQAPRLLIYGASSRATGIPDRFSGSG |
| | | SGTDFTLTISRLEPEDFAVYYCQHYGSSPSWTFGQGTRLEIKTTTP |
| | | APRPPTPAPTIASQPLSLRPEACRPAAGGAVHTRGLDFACDIYIW |
| | | APLAGTCGVLLLSLVITLYCKRGRKKLLYIFKQPFMRPVQTTQEE |
| | | DGCSCRFPEEEEGGCELRVKFSRSADAPAYKQGQNQLYNELNLG |
| | | RREEYDVLDKRRGRDPEMGGKPRRKNPQEGLYNELQKDKMAE |
| | | AYSEIGMKGERRRGKGHDGLYQGLSTATKDTYDALHMQALPPR |
| BCMA_EBB- | 1586 | ATGGCCCTCCCTGTCACCGCCCTGCTGCTTCCGCTGGCTCTTC |
| C1980- | | TGCTCCACGCCGCTCGGCCCGAAGTGCAGCTGCTGGAGTCCG |
| D2-nt | | GCGGTGGATTGGTGCAACCGGGGGGATCGCTCAGACTGTCCT |
| Full CART | | GTGCGGCGTCAGGCTTCACCTTCTCGAGCTACGCCATGTCATG |
| | | GGTCAGACAGGCCCCTGGAAAGGGTCTGGAATGGGTGTCCGC |
| | | CATTTCCGGGAGCGGGGGATCTACATACTACGCCGATAGCGT |
| | | GAAGGGCCGCTTCACCATTTCCCGGGACAACTCCAAGAACAC |
| | | TCTCTATCTGCAAATGAACTCCCTCCGCGCTGAGGACACTGCC |
| | | GTGTACTACTGCGCCAAAATCCCTCAGACCGGCACCTTCGACT |
| | | ACTGGGGACAGGGGACTCTGGTCACCGTCAGCAGCGGTGGCG |
| | | GAGGTTCGGGGGGAGGAGGAAGCGGCGGCGGAGGGTCCGAG |
| | | ATTGTGCTGACCCAGTCACCCGGCACTTTGTCCCTGTCGCCTG |
| | | GAGAAAGGGCCACCCTTTCCTGCCGGGCATCCCAATCCGTGT |
| | | CCTCCTCGTACCTGGCCTGGTACCAGCAGAGGCCCGGACAGG |
| | | CCCCACGGCTTCTGATCTACGGAGCAAGCAGCCGCGCGACCG |
| | | GTATCCCGGACCGGTTTTCGGGCTCGGGCTCAGGAACTGACTT |
| | | CACCCTCACCATCTCCCGCCTGGAACCCGAAGATTTCGCTGTG |
| | | TATTACTGCCAGCACTACGGCAGCTCCCCGTCCTGGACGTTCG |
| | | GCCAGGGAACTCGGCTGGAGATCAAGACCACTACCCCAGCAC |
| | | CGAGGCCACCCACCCCGGCTCCTACCATCGCCTCCCAGCCTCT |
| | | GTCCCTGCGTCCGGAGGCATGTAGACCCGCAGCTGGTGGGGC |
| | | CGTGCATACCCGGGGTCTTGACTTCGCCTGCGATATCTACATT |
| | | TGGGCCCCTCTGGCTGGTACTTGCGGGGTCCTGCTGCTTTCAC |
| | | TCGTGATCACTCTTTACTGTAAGCGCGGTCGGAAGAAGCTGCT |
| | | GTACATCTTTAAGCAACCCTTCATGAGGCCTGTGCAGACTACT |
| | | CAAGAGGAGGACGGCTGTTCATGCCGGTTCCCAGAGGAGGAG |
| | | GAAGGCGGCTGCGAACTGCGCGTGAAATTCAGCCGCAGCGCA |
| | | GATGCTCCAGCCTACAAGCAGGGGCAGAACCAGCTCTACAAC |
| | | GAACTCAATCTTGGTCGGAGAGAGGAGTACGACGTGCTGGAC |
| | | AAGCGGAGAGGACGGGACCCAGAAATGGGCGGGAAGCCGCG |
| | | CAGAAAGAATCCCCAAGAGGGCCTGTACAACGAGCTCCAAA |
| | | AGGATAAGATGGCAGAAGCCTATAGCGAGATTGGTATGAAA |
| | | GGGGAACGCAGAAGAGGCAAAGGCCACGACGGACTGTACCA |
| | | GGGACTCAGCACCGCCACCAAGGACACCTATGACGCTCTTCA |
| | | CATGCAGGCCCTGCCGCCTCGG |

BCMA_EBB-C1978-A10

| | | |
|---|---|---|
| BCMA_EBB- | 1587 | EVQLVETGGGLVQPGGSLRLSCAASGFTFSSYAMSWVRQAPGK |
| C1978- | | GLEWVSAISGSGGSTYYADSVKGRFTMSRENDKNSVFLQMNSL |
| A10-aa | | RVEDTGVYYCARANYKRELRYYYGMDVWGQGTMVTVSSGGG |
| ScFv | | GSGGGGSGGGGSEIVMTQSPGTLSLSPGESATLSCRASQRVASN |
| domain | | YLAWYQHKPGQAPSLLISGASSRATGVPDRFSGSGSGTDFTLAIS |
| | | RLEPEDSAVYYCQHYDSSPSWTFGQGTKVEIK |
| BCMA_EBB- | 1588 | GAAGTGCAACTGGTGGAAACCGGTGGAGGACTCGTGCAGCCT |
| C1978- | | GGCGGCAGCCTCCGGCTGAGCTGCGCCGCTTCGGGATTCACC |
| A10-nt | | TTTTCCTCCTACGCGATGTCTTGGGTCAGACAGGCCCCCGGAA |
| ScFv | | AGGGGCTGGAATGGGTGTCAGCCATCTCCGGCTCCGGCGGAT |
| domain | | CAACGTACTACGCCGACTCCGTGAAAGGCCGGTTCACCATGT |
| | | CGCGCGAGAATGACAAGAACTCCGTGTTCCTGCAAATGAACT |
| | | CCCTGAGGGTGGAGGACACCGGAGTGTACTATTGTGCGCGCG |
| | | CCAACTACAAGAGAGAGCTGCGGTACTACTACGGAATGGACG |
| | | TCTGGGGACAGGGAACTATGGTGACCGTGTCATCCGGTGGAG |
| | | GGGGAAGCGGCGGTGGAGGCAGCGGGGGCGGGGGTTCAGAA |
| | | ATTGTCATGACCCAGTCCCCGGGAACTCTTTCCCTCTCCCCCG |
| | | GGGAATCCGCGACTTTGTCCTGCCGGGCCAGCCAGCGCGTGG |
| | | CCTCGAACTACCTCGCATGGTACCAGCATAAGCCAGGCCAAG |
| | | CCCCTTCCCTGCTGATTTCCGGGGCTAGCAGCCGCGCCACTGG |
| | | CGTGCCGGATAGGTTCTCGGGAAGCGGCTCGGGTACCGATTT |
| | | CACCCTGGCAATCTCGCGGCTGGAACCGGAGGATTCGGCCGT |
| | | GTACTACTGCCAGCACTATGACTCATCCCCCTCCTGGACATTC |
| | | GGACAGGGCACCAAGGTCGAGATCAAG |
| BCMA_EBB- | 1589 | EVQLVETGGGLVQPGGSLRLSCAASGFTFSSYAMSWVRQAPGK |
| C1978- | | GLEWVSAISGSGGSTYYADSVKGRFTMSRENDKNSVFLQMNSL |
| A10-aa | | RVEDTGVYYCARANYKRELRYYYGMDVWGQGTMVTVSS |
| VH | | |
| BCMA_EBB- | 1590 | EIVMTQSPGTLSLSPGESATLSCRASQRVASNYLAWYQHKPGQA |
| C1978- | | PSLLISGASSRATGVPDRFSGSGSGTDFTLAISRLEPEDSAVYYCQ |
| A10-aa | | HYDSSPSWTFGQGTKVEIK |
| VL | | |
| BCMA_EBB- | 1591 | MALPVTALLLPLALLLHAARPEVQLVETGGGLVQPGGSLRLSCA |
| C1978- | | ASGFTFSSYAMSWVRQAPGKGLEWVSAISGSGGSTYYADSVKG |
| A10-aa | | RFTMSRENDKNSVFLQMNSLRVEDTGVYYCARANYKRELRYY |
| Full CART | | YGMDVWGQGTMVTVSSGGGGSGGGGSGGGGSEIVMTQSPGTL |
| | | SLSPGESATLSCRASQRVASNYLAWYQHKPGQAPSLLISGASSRA |
| | | TGVPDRFSGSGSGTDFTLAISRLEPEDSAVYYCQHYDSSPSWTFG |
| | | QGTKVEIKTTTPAPRPPTPAPTIASQPLSLRPEACRPAAGGAVHTR |
| | | GLDFACDIYIWAPLAGTCGVLLLSLVITLYCKRGRKKLLYIFKQP |
| | | FMRPVQTTQEEDGCSCRFPEEEEGGCELRVKFSRSADAPAYKQG |
| | | QNQLYNELNLGRREEYDVLDKRRGRDPEMGGKPRRKNPQEGL |
| | | YNELQKDKMAEAYSEIGMKGERRRGKGHDGLYQGLSTATKDT |
| | | YDALHMQALPPR |
| BCMA_EBB- | 1592 | ATGGCCCTCCCTGTCACCGCCCTGCTGCTTCCGCTGGCTCTTC |
| C1978- | | TGCTCCACGCCGCTCGGCCCGAAGTGCAACTGGTGGAAACCG |
| A10-nt | | GTGGAGGACTCGTGCAGCCTGGCGGCAGCCTCCGGCTGAGCT |
| Full CART | | GCGCCGCTTCGGGATTCACCTTTTCCTCCTACGCGATGTCTTG |
| | | GGTCAGACAGGCCCCCGGAAAGGGGCTGGAATGGGTGTCAG |
| | | CCATCTCCGGCTCCGGCGGATCAACGTACTACGCCGACTCCGT |
| | | GAAAGGCCGGTTCACCATGTCGCGCGAGAATGACAAGAACTC |
| | | CGTGTTCCTGCAAATGAACTCCCTGAGGGTGGAGGACACCGG |
| | | AGTGTACTATTGTGCGCGCGCCAACTACAAGAGAGAGCTGCG |
| | | GTACTACTACGGAATGGACGTCTGGGGACAGGGAACTATGGT |
| | | GACCGTGTCATCCGGTGGAGGGGGAAGCGGCGGTGGAGGCA |
| | | GCGGGGGCGGGGGTTCAGAAATTGTCATGACCCAGTCCCCGG |
| | | GAACTCTTTCCCTCTCCCCCGGGGAATCCGCGACTTTGTCCTG |
| | | CCGGGCCAGCCAGCGCGTGGCCTCGAACTACCTCGCATGGTA |
| | | CCAGCATAAGCCAGGCCAAGCCCCTTCCCTGCTGATTTCCGG |
| | | GGCTAGCAGCCGCGCCACTGGCGTGCCGGATAGGTTCTCGGG |
| | | AAGCGGCTCGGGTACCGATTTCACCCTGGCAATCTCGCGGCT |
| | | GGAACCGGAGGATTCGGCCGTGTACTACTGCCAGCACTATGA |
| | | CTCATCCCCCTCCTGGACATTCGGACAGGGCACCAAGGTCGA |
| | | GATCAAGACCACTACCCCAGCACCGAGGCCACCCACCCCGGC |
| | | TCCTACCATCGCCTCCCAGCCTCTGTCCCTGCGTCCGGAGGCA |
| | | TGTAGACCCGCAGCTGGTGGGGCCGTGCATACCCGGGGTCTT |
| | | GACTTCGCCTGCGATATCTACATTTGGGCCCCTCTGGCTGGTA |
| | | CTTGCGGGGTCCTGCTGCTTTCACTCGTGATCACTCTTTACTGT |
| | | AAGCGCGGTCGGAAGAAGCTGCTGTACATCTTTAAGCAACCC |
| | | TTCATGAGGCCTGTGCAGACTACTCAAGAGGAGGACGGCTGT |
| | | TCATGCCGGTTCCCAGAGGAGGAGGAAGGCGGCTGCGAACTG |
| | | CGCGTGAAATTCAGCCGCAGCGCAGATGCTCCAGCCTACAAG |
| | | CAGGGGCAGAACCAGCTCTACAACGAACTCAATCTTGGTCGG |
| | | AGAGAGGAGTACGACGTGCTGGACAAGCGGAGAGGACGGGA |
| | | CCCAGAAATGGGCGGGAAGCCGCGCAGAAAGAATCCCCAAG |
| | | AGGGCCTGTACAACGAGCTCCAAAAGGATAAGATGGCAGAA |
| | | GCCTATAGCGAGATTGGTATGAAAGGGGAACGCAGAAGAGG |
| | | CAAAGGCCACGACGGACTGTACCAGGGACTCAGCACCGCCAC |
| | | CAAGGACACCTATGACGCTCTTCACATGCAGGCCCTGCCGCC |
| | | TCGG |

BCMA_EBB-C1978-D4

| | | |
|---|---|---|
| BCMA_EBB- | 1593 | EVQLLETGGGLVQPGGSLRLSCAASGFSFSSYAMSWVRQAPGK |
| C1978- | | GLEWVSAISGSGGSTYYADSVKGRFTISRDNSKNTLYLQMNSLR |
| D4-aa | | AEDTAVYYCAKALVGATGAFDIWGQGTLVTVSSGGGGSGGGG |
| ScFv | | SGGGGSEIVLTQSPGTLSLSPGERATLSCRASQSLSSNFLAWYQQ |
| domain | | KPGQAPGLLIYGASNWATGTPDRFSGSGSGTDFTLTITRLEPEDF |
| | | AVYYCQYYGTSPMYTFGQGTKVEIK |
| BCMA_EBB- | 1594 | GAAGTGCAGCTGCTCGAAACCGGTGGAGGGCTGGTGCAGCCA |
| C1978- | | GGGGGCTCCCTGAGGCTTTCATGCGCCGCTAGCGGATTCTCCT |
| D4-nt | | TCTCCTCTTACGCCATGTCGTGGGTCCGCCAAGCCCCTGGAAA |
| ScFv | | AGGCCTGGAATGGGTGTCCGCGATTTCCGGGAGCGGAGGTTC |
| domain | | GACCTATTACGCCGACTCCGTGAAGGGCCGCTTTACCATCTCC |
| | | CGGGATAACTCCAAGAACACTCTGTACCTCCAAATGAACTCG |
| | | CTGAGAGCCGAGGACACCGCCGTGTATTACTGCGCGAAGGCG |
| | | CTGGTCGGCGCGACTGGGGCATTCGACATCTGGGGACAGGGA |
| | | ACTCTTGTGACCGTGTCGAGCGGAGGCGGCGGCTCCGGCGGA |
| | | GGAGGGAGCGGGGGCGGTGGTTCCGAAATCGTGTTGACTCAG |
| | | TCCCCGGGAACCCTGAGCTTGTCACCCGGGGAGCGGGCCACT |
| | | CTCTCCTGTCGCGCCTCCCAATCGCTCTCATCCAATTTCCTGG |
| | | CCTGGTACCAGCAGAAGCCCGGACAGGCCCCGGGCCTGCTCA |
| | | TCTACGGCGCTTCAAACTGGGCAACGGGAACCCCTGATCGGT |
| | | TCAGCGGAAGCGGATCGGGTACTGACTTTACCCTGACCATCA |
| | | CCAGACTGGAACCGGAGGACTTCGCCGTGTACTACTGCCAGT |
| | | ACTACGGCACCTCCCCCATGTACACATTCGGACAGGGTACCA |
| | | AGGTCGAGATTAAG |
| BCMA_EBB- | 1595 | EVQLLETGGGLVQPGGSLRLSCAASGFSFSSYAMSWVRQAPGK |
| C1978- | | GLEWVSAISGSGGSTYYADSVKGRFTISRDNSKNTLYLQMNSLR |
| D4-aa | | AEDTAVYYCAKALVGATGAFDIWGQGTLVTVSS |
| VH | | |
| BCMA_EBB- | 1596 | EIVLTQSPGTLSLSPGERATLSCRASQSLSSNFLAWYQQKPGQAP |
| C1978- | | GLLIYGASNWATGTPDRFSGSGSGTDFTLTITRLEPEDFAVYYCQ |
| D4-aa | | YYGTSPMYTFGQGTKVEIK |
| VL | | |
| BCMA_EBB- | 1597 | MALPVTALLLPLALLLHAARPEVQLLETGGGLVQPGGSLRLSCA |
| C1978- | | ASGFSFSSYAMSWVRQAPGKGLEWVSAISGSGGSTYYADSVKG |
| D4-aa | | RFTISRDNSKNTLYLQMNSLRAEDTAVYYCAKALVGATGAFDI |
| Full CART | | WGQGTLVTVSSGGGGSGGGGSGGGGSEIVLTQSPGTLSLSPGER |
| | | ATLSCRASQSLSSNFLAWYQQKPGQAPGLLIYGASNWATGTPDR |
| | | FSGSGSGTDFTLTITRLEPEDFAVYYCQYYGTSPMYTFGQGTKVE |
| | | IKTTTPAPRPPTPAPTIASQPLSLRPEACRPAAGGAVHTRGLDFAC |
| | | DIYIWAPLAGTCGVLLLSLVITLYCKRGRKKLLYIFKQPFMRPVQ |
| | | TTQEEDGCSCRFPEEEEGGCELRVKFSRSADAPAYKQGQNQLYN |
| | | ELNLGRREEYDVLDKRRGRDPEMGGKPRRKNPQEGLYNELQKD |
| | | KMAEAYSEIGMKGERRRGKGHDGLYQGLSTATKDTYDALHMQ |
| | | ALPPR |
| BCMA_EBB- | 1598 | ATGGCCCTCCCTGTCACCGCCCTGCTGCTTCCGCTGGCTCTTC |
| C1978- | | TGCTCCACGCCGCTCGGCCCGAAGTGCAGCTGCTCGAAACCG |
| D4-nt | | GTGGAGGGCTGGTGCAGCCAGGGGGCTCCCTGAGGCTTTCAT |
| Full CART | | GCGCCGCTAGCGGATTCTCCTTCTCCTCTTACGCCATGTCGTG |
| | | GGTCCGCCAAGCCCCTGGAAAAGGCCTGGAATGGGTGTCCGC |
| | | GATTTCCGGGAGCGGAGGTTCGACCTATTACGCCGACTCCGT |
| | | GAAGGGCCGCTTTACCATCTCCCGGGATAACTCCAAGAACAC |
| | | TCTGTACCTCCAAATGAACTCGCTGAGAGCCGAGGACACCGC |
| | | CGTGTATTACTGCGCGAAGGCGCTGGTCGGCGCGACTGGGGC |
| | | ATTCGACATCTGGGGACAGGGAACTCTTGTGACCGTGTCGAG |
| | | CGGAGGCGGCGGCTCCGGCGGAGGAGGGAGCGGGGGCGGTG |
| | | GTTCCGAAATCGTGTTGACTCAGTCCCCGGGAACCCTGAGCTT |
| | | GTCACCCGGGGAGCGGGCCACTCTCTCCTGTCGCGCCTCCCA |
| | | ATCGCTCTCATCCAATTTCCTGGCCTGGTACCAGCAGAAGCCC |
| | | GGACAGGCCCCGGGCCTGCTCATCTACGGCGCTTCAAACTGG |
| | | GCAACGGGAACCCCTGATCGGTTCAGCGGAAGCGGATCGGGT |
| | | ACTGACTTTACCCTGACCATCACCAGACTGGAACCGGAGGAC |
| | | TTCGCCGTGTACTACTGCCAGTACTACGGCACCTCCCCCATGT |
| | | ACACATTCGGACAGGGTACCAAGGTCGAGATTAAGACCACTA |
| | | CCCCAGCACCGAGGCCACCCACCCCGGCTCCTACCATCGCCT |
| | | CCCAGCCTCTGTCCCTGCGTCCGGAGGCATGTAGACCCGCAG |
| | | CTGGTGGGGCCGTGCATACCCGGGGTCTTGACTTCGCCTGCG |
| | | ATATCTACATTTGGGCCCCTCTGGCTGGTACTTGCGGGGTCCT |
| | | GCTGCTTTCACTCGTGATCACTCTTTACTGTAAGCGCGGTCGG |
| | | AAGAAGCTGCTGTACATCTTTAAGCAACCCTTCATGAGGCCT |
| | | GTGCAGACTACTCAAGAGGAGGACGGCTGTTCATGCCGGTTC |
| | | CCAGAGGAGGAGGAAGGCGGCTGCGAACTGCGCGTGAAATT |
| | | CAGCCGCAGCGCAGATGCTCCAGCCTACAAGCAGGGGCAGA |
| | | ACCAGCTCTACAACGAACTCAATCTTGGTCGGAGAGAGGAGT |
| | | ACGACGTGCTGGACAAGCGGAGAGGACGGGACCCAGAAATG |
| | | GGCGGGAAGCCGCGCAGAAAGAATCCCCAAGAGGGCCTGTA |
| | | CAACGAGCTCCAAAAGGATAAGATGGCAGAAGCCTATAGCG |
| | | AGATTGGTATGAAAGGGGAACGCAGAAGAGGCAAAGGCCAC |
| | | GACGGACTGTACCAGGGACTCAGCACCGCCACCAAGGACACC |
| | | TATGACGCTCTTCACATGCAGGCCCTGCCGCCTCGG |

BCMA_EBB-C1980-A2

| | | |
|---|---|---|
| BCMA_EBB- | 1599 | EVQLLESGGGLVQPGGSLRLSCAASGFTFSSYAMSWVRQAPGK |
| C1980- | | GLEWVSAISGSGGSTYYADSVKGRFTISRDNSKNTLYLQMNSLR |
| A2-aa | | AEDTAVYYCVLWFGEGFDPWGQGTLVTVSSGGGGSGGGGSGG |
| ScFv | | GGSDIVLTQSPLSLPVTPGEPASISCRSSQSLLHSNGYNYLDWYL |
| domain | | QKPGQSPQLLIYLGSNRASGVPDRFSGSGSGTDFTLKISRVEAED |
| | | VGVYYCMQALQTPLTFGGGTKVDIK |
| BCMA_EBB- | 1600 | GAAGTGCAGCTGCTTGAGAGCGGTGGAGGTCTGGTGCAGCCC |
| C1980- | | GGGGGATCACTGCGCCTGTCCTGTGCCGCGTCCGGTTTCACTT |
| A2-nt | | TCTCCTCGTACGCCATGTCGTGGGTCAGACAGGCACCGGGAA |
| ScFv | | AGGGACTGGAATGGGTGTCAGCCATTTCGGGTTCGGGGGGCA |
| domain | | GCACCTACTACGCTGACTCCGTGAAGGGCCGGTTCACCATTTC |
| | | CCGCGACAACTCCAAGAACACCTTGTACCTCCAAATGAACTC |
| | | CCTGCGGGCCGAAGATACCGCCGTGTATTACTGCGTGCTGTG |
| | | GTTCGGAGAGGGATTCGACCCGTGGGGACAAGGAACACTCGT |
| | | GACTGTGTCATCCGGCGGAGGCGGCAGCGGTGGCGGCGGTTC |
| | | CGGCGGCGGCGGATCTGACATCGTGTTGACCCAGTCCCCTCT |
| | | GAGCCTGCCGGTCACTCCTGGCGAACCAGCCAGCATCTCCTG |
| | | CCGGTCGAGCCAGTCCCTCCTGCACTCCAATGGGTACAACTA |
| | | CCTCGATTGGTATCTGCAAAAGCCGGGCCAGAGCCCCCAGCT |
| | | GCTGATCTACCTTGGGTCAAACCGCGCTTCCGGGGTGCCTGAT |
| | | AGATTCTCCGGGTCCGGGAGCGGAACCGACTTTACCCTGAAA |
| | | ATCTCGAGGGTGGAGGCCGAGGACGTCGGAGTGTACTACTGC |
| | | ATGCAGGCGCTCCAGACTCCCCTGACCTTCGGAGGAGGAACG |
| | | AAGGTCGACATCAAGA |
| BCMA_EBB- | 1601 | EVQLLESGGGLVQPGGSLRLSCAASGFTFSSYAMSWVRQAPGK |
| C1980- | | GLEWVSAISGSGGSTYYADSVKGRFTISRDNSKNTLYLQMNSLR |
| A2-aa | | AEDTAVYYCVLWFGEGFDPWGQGTLVTVSS |
| VH | | |
| BCMA_EBB- | 1602 | DIVLTQSPLSLPVTPGEPASISCRSSQSLLHSNGYNYLDWYLQKP |
| C1980- | | GQSPQLLIYLGSNRASGVPDRFSGSGSGTDFTLKISRVEAEDVGV |
| A2-aa | | YYCMQALQTPLTFGGGTKVDIK |
| VL | | |
| BCMA_EBB- | 1603 | MALPVTALLLPLALLLHAARPEVQLLESGGGLVQPGGSLRLSCA |
| C1980- | | ASGFTFSSYAMSWVRQAPGKGLEWVSAISGSGGSTYYADSVKG |
| A2-aa | | RFTISRDNSKNTLYLQMNSLRAEDTAVYYCVLWFGEGFDPWGQ |
| Full CART | | GTLVTVSSGGGGSGGGGSGGGGSDIVLTQSPLSLPVTPGEPASIS |
| | | CRSSQSLLHSNGYNYLDWYLQKPGQSPQLLIYLGSNRASGVPDR |
| | | FSGSGSGTDFTLKISRVEAEDVGVYYCMQALQTPLTFGGGTKVD |
| | | IKTTTPAPRPPTPAPTIASQPLSLRPEACRPAAGGAVHTRGLDFAC |
| | | DIYIWAPLAGTCGVLLLSLVITLYCKRGRKKLLYIFKQPFMRPVQ |
| | | TTQEEDGCSCRFPEEEEGGCELRVKFSRSADAPAYKQGQNQLYN |
| | | ELNLGRREEYDVLDKRRGRDPEMGGKPRRKNPQEGLYNELQKD |
| | | KMAEAYSEIGMKGERRRGKGHDGLYQGLSTATKDTYDALHMQ |
| | | ALPPR |
| BCMA_EBB- | 1604 | ATGGCCCTCCCTGTCACCGCCCTGCTGCTTCCGCTGGCTCTTC |
| C1980- | | TGCTCCACGCCGCTCGGCCCGAAGTGCAGCTGCTTGAGAGCG |
| A2-nt | | GTGGAGGTCTGGTGCAGCCCGGGGGATCACTGCGCCTGTCCT |
| Full CART | | GTGCCGCGTCCGGTTTCACTTTCTCCTCGTACGCCATGTCGTG |
| | | GGTCAGACAGGCACCGGGAAAGGGACTGGAATGGGTGTCAG |
| | | CCATTTCGGGTTCGGGGGGCAGCACCTACTACGCTGACTCCGT |
| | | GAAGGGCCGGTTCACCATTTCCCGCGACAACTCCAAGAACAC |
| | | CTTGTACCTCCAAATGAACTCCCTGCGGGCCGAAGATACCGC |
| | | CGTGTATTACTGCGTGCTGTGGTTCGGAGAGGGATTCGACCC |
| | | GTGGGGACAAGGAACACTCGTGACTGTGTCATCCGGCGGAGG |
| | | CGGCAGCGGTGGCGGCGGTTCCGGCGGCGGCGGATCTGACAT |
| | | CGTGTTGACCCAGTCCCCTCTGAGCCTGCCGGTCACTCCTGGC |
| | | GAACCAGCCAGCATCTCCTGCCGGTCGAGCCAGTCCCTCCTG |
| | | CACTCCAATGGGTACAACTACCTCGATTGGTATCTGCAAAAG |
| | | CCGGGCCAGAGCCCCCAGCTGCTGATCTACCTTGGGTCAAAC |
| | | CGCGCTTCCGGGGTGCCTGATAGATTCTCCGGGTCCGGGAGC |
| | | GGAACCGACTTTACCCTGAAAATCTCGAGGGTGGAGGCCGAG |
| | | GACGTCGGAGTGTACTACTGCATGCAGGCGCTCCAGACTCCC |
| | | CTGACCTTCGGAGGAGGAACGAAGGTCGACATCAAGACCACT |
| | | ACCCCAGCACCGAGGCCACCCACCCCGGCTCCTACCATCGCC |
| | | TCCCAGCCTCTGTCCCTGCGTCCGGAGGCATGTAGACCCGCA |
| | | GCTGGTGGGGCCGTGCATACCCGGGGTCTTGACTTCGCCTGC |
| | | GATATCTACATTTGGGCCCCTCTGGCTGGTACTTGCGGGGTCC |
| | | TGCTGCTTTCACTCGTGATCACTCTTTACTGTAAGCGCGGTCG |
| | | GAAGAAGCTGCTGTACATCTTTAAGCAACCCTTCATGAGGCC |
| | | TGTGCAGACTACTCAAGAGGAGGACGGCTGTTCATGCCGGTT |
| | | CCCAGAGGAGGAGGAAGGCGGCTGCGAACTGCGCGTGAAAT |
| | | TCAGCCGCAGCGCAGATGCTCCAGCCTACAAGCAGGGGCAGA |
| | | ACCAGCTCTACAACGAACTCAATCTTGGTCGGAGAGAGGAGT |
| | | ACGACGTGCTGGACAAGCGGAGAGGACGGGACCCAGAAATG |
| | | GGCGGGAAGCCGCGCAGAAAGAATCCCCAAGAGGGCCTGTA |
| | | CAACGAGCTCCAAAAGGATAAGATGGCAGAAGCCTATAGCG |
| | | AGATTGGTATGAAAGGGGAACGCAGAAGAGGCAAAGGCCAC |
| | | GACGGACTGTACCAGGGACTCAGCACCGCCACCAAGGACACC |
| | | TATGACGCTCTTCACATGCAGGCCCTGCCGCCTCGG |

BCMA_EBB-C1981-C3

| | | |
|---|---|---|
| BCMA_EBB- | 1605 | QVQLVESGGGLVQPGGSLRLSCAASGFTFSSYAMSWVRQAPGK |
| C1981- | | GLEWVSAISGSGGSTYYADSVKGRFTISRDNSKNTLYLQMNSLR |
| C3-aa | | AEDTAVYYCAKVGYDSSGYYRDYYGMDVWGQGTTVTVSSGG |
| ScFv | | GGSGGGGSGGGGSEIVLTQSPGTLSLSPGERATLSCRASQSVSSS |
| domain | | YLAWYQQKPGQAPRLLIYGTSSRATGISDRFSGSGSGTDFTLTIS |
| | | RLEPEDFAVYYCQHYGNSPPKFTFGPGTKLEIK |
| BCMA_EBB- | 1606 | CAAGTGCAGCTCGTGGAGTCAGGCGGAGGACTGGTGCAGCCC |
| C1981- | | GGGGGCTCCCTGAGACTTTCCTGCGCGGCATCGGGTTTTACCT |
| C3-nt | | TCTCCTCCTATGCTATGTCCTGGGTGCGCCAGGCCCCGGGAAA |
| ScFv | | GGGACTGGAATGGGTGTCCGCAATCAGCGGTAGCGGGGGCTC |
| domain | | AACATACTACGCCGACTCCGTCAAGGGTCGCTTCACTATTTCC |
| | | CGGGACAACTCCAAGAATACCCTGTACCTCCAAATGAACAGC |
| | | CTCAGGGCCGAGGATACTGCCGTGTACTACTGCGCCAAAGTC |
| | | GGATACGATAGCTCCGGTTACTACCGGGACTACTACGGAATG |
| | | GACGTGTGGGGACAGGGCACCACCGTGACCGTGTCAAGCGGC |
| | | GGAGGCGGTTCAGGAGGGGGAGGCTCCGGCGGTGGAGGGTC |
| | | CGAAATCGTCCTGACTCAGTCGCCTGGCACTCTGTCGTTGTCC |
| | | CCGGGGGAGCGCGCTACCCTGTCGTGTCGGGCGTCGCAGTCC |
| | | GTGTCGAGCTCCTACCTCGCGTGGTACCAGCAGAAGCCCGGA |
| | | CAGGCCCCTAGACTTCTGATCTACGGCACTTCTTCACGCGCCA |
| | | CCGGGATCAGCGACAGGTTCAGCGGCTCCGGCTCCGGGACCG |
| | | ACTTCACCCTGACCATTAGCCGGCTGGAGCCTGAAGATTTCGC |
| | | CGTGTATTACTGCCAACACTACGGAAACTCGCCGCCAAAGTT |
| | | CACGTTCGGACCCGGAACCAAGCTGGAAATCAAG |
| BCMA_EBB- | 1607 | QVQLVESGGGLVQPGGSLRLSCAASGFTFSSYAMSWVRQAPGK |
| C1981- | | GLEWVSAISGSGGSTYYADSVKGRFTISRDNSKNTLYLQMNSLR |
| C3-aa | | AEDTAVYYCAKVGYDSSGYYRDYYGMDVWGQGTTVTVSS |
| VH | | |
| BCMA_EBB- | 1608 | EIVLTQSPGTLSLSPGERATLSCRASQSVSSSYLAWYQQKPGQAP |
| C1981- | | RLLIYGTSSRATGISDRFSGSGSGTDFTLTISRLEPEDFAVYYCQH |
| C3-aa | | YGNSPPKFTFGPGTKLEIK |
| VL | | |
| BCMA_EBB- | 1609 | MALPVTALLLPLALLLHAARPQVQLVESGGGLVQPGGSLRLSCA |
| C1981- | | ASGFTFSSYAMSWVRQAPGKGLEWVSAISGSGGSTYYADSVKG |
| C3-aa | | RFTISRDNSKNTLYLQMNSLRAEDTAVYYCAKVGYDSSGYYRD |
| Full CART | | YYGMDVWGQGTTVTVSSGGGGSGGGGSGGGGSEIVLTQSPGTL |
| | | SLSPGERATLSCRASQSVSSSYLAWYQQKPGQAPRLLIYGTSSRA |
| | | TGISDRFSGSGSGTDFTLTISRLEPEDFAVYYCQHYGNSPPKFTFG |
| | | PGTKLEIKTTTPAPRPPTPAPTIASQPLSLRPEACRPAAGGAVHTR |
| | | GLDFACDIYIWAPLAGTCGVLLLSLVITLYCKRGRKKLLYIFKQP |
| | | FMRPVQTTQEEDGCSCRFPEEEEGGCELRVKFSRSADAPAYKQG |
| | | QNQLYNELNLGRREEYDVLDKRRGRDPEMGGKPRRKNPQEGL |
| | | YNELQKDKMAEAYSEIGMKGERRRGKGHDGLYQGLSTATKDT |
| | | YDALHMQALPPR |
| BCMA_EBB- | 1610 | ATGGCCCTCCCTGTCACCGCCCTGCTGCTTCCGCTGGCTCTTC |
| C1981- | | TGCTCCACGCCGCTCGGCCCCAAGTGCAGCTCGTGGAGTCAG |
| C3-nt | | GCGGAGGACTGGTGCAGCCCGGGGGCTCCCTGAGACTTTCCT |
| Full CART | | GCGCGGCATCGGGTTTTACCTTCTCCTCCTATGCTATGTCCTG |
| | | GGTGCGCCAGGCCCCGGGAAAGGGACTGGAATGGGTGTCCGC |
| | | AATCAGCGGTAGCGGGGGCTCAACATACTACGCCGACTCCGT |
| | | CAAGGGTCGCTTCACTATTTCCCGGGACAACTCCAAGAATAC |
| | | CCTGTACCTCCAAATGAACAGCCTCAGGGCCGAGGATACTGC |
| | | CGTGTACTACTGCGCCAAAGTCGGATACGATAGCTCCGGTTA |
| | | CTACCGGGACTACTACGGAATGGACGTGTGGGGACAGGGCAC |
| | | CACCGTGACCGTGTCAAGCGGCGGAGGCGGTTCAGGAGGGG |
| | | GAGGCTCCGGCGGTGGAGGGTCCGAAATCGTCCTGACTCAGT |
| | | CGCCTGGCACTCTGTCGTTGTCCCCGGGGGAGCGCGCTACCCT |
| | | GTCGTGTCGGGCGTCGCAGTCCGTGTCGAGCTCCTACCTCGCG |
| | | TGGTACCAGCAGAAGCCCGGACAGGCCCCTAGACTTCTGATC |
| | | TACGGCACTTCTTCACGCGCCACCGGGATCAGCGACAGGTTC |
| | | AGCGGCTCCGGCTCCGGGACCGACTTCACCCTGACCATTAGC |
| | | CGGCTGGAGCCTGAAGATTTCGCCGTGTATTACTGCCAACACT |
| | | ACGGAAACTCGCCGCCAAAGTTCACGTTCGGACCCGGAACCA |
| | | AGCTGGAAATCAAGACCACTACCCCAGCACCGAGGCCACCCA |
| | | CCCCGGCTCCTACCATCGCCTCCCAGCCTCTGTCCCTGCGTCC |
| | | GGAGGCATGTAGACCCGCAGCTGGTGGGGCCGTGCATACCCG |
| | | GGGTCTTGACTTCGCCTGCGATATCTACATTTGGGCCCCTCTG |
| | | GCTGGTACTTGCGGGGTCCTGCTGCTTTCACTCGTGATCACTC |
| | | TTTACTGTAAGCGCGGTCGGAAGAAGCTGCTGTACATCTTTAA |
| | | GCAACCCTTCATGAGGCCTGTGCAGACTACTCAAGAGGAGGA |
| | | CGGCTGTTCATGCCGGTTCCCAGAGGAGGAGGAAGGCGGCTG |
| | | CGAACTGCGCGTGAAATTCAGCCGCAGCGCAGATGCTCCAGC |
| | | CTACAAGCAGGGGCAGAACCAGCTCTACAACGAACTCAATCT |
| | | TGGTCGGAGAGAGGAGTACGACGTGCTGGACAAGCGGAGAG |
| | | GACGGGACCCAGAAATGGGCGGGAAGCCGCGCAGAAAGAAT |
| | | CCCCAAGAGGGCCTGTACAACGAGCTCCAAAAGGATAAGATG |
| | | GCAGAAGCCTATAGCGAGATTGGTATGAAAGGGGAACGCAG |
| | | AAGAGGCAAAGGCCACGACGGACTGTACCAGGGACTCAGCA |
| | | CCGCCACCAAGGACACCTATGACGCTCTTCACATGCAGGCCC |
| | | TGCCGCCTCGG |

BCMA_EBB-C1978-G4

| | | |
|---|---|---|
| BCMA_EBB- | 1611 | EVQLVESGGGLVQPGGSLRLSCAASGFTFSSYAMSWVRQAPGK |
| C1978- | | GLEWVSAISGSGGSTYYADSVKGRFTISRDNSKNTLYLQMNSLR |
| G4-aa | | AEDTAVYYCAKMGWSSGYLGAFDIWGQGTTVTVSSGGGGSGG |
| ScFv | | GGSGGGGSEIVLTQSPGTLSLSPGERATLSCRASQSVASSFLAWY |
| domain | | QQKPGQAPRLLIYGASGRATGIPDRFSGSGSGTDFTLTISRLEPED |
| | | FAVYYCQHYGGSPRLTFGGGTKVDIK |
| BCMA_EBB- | 1612 | GAAGTCCAACTGGTGGAGTCCGGGGGAGGGCTCGTGCAGCCC |
| C1978- | | GGAGGCAGCCTTCGGCTGTCGTGCGCCGCCTCCGGGTTCACG |
| G4-nt | | TTCTCATCCTACGCGATGTCGTGGGTCAGACAGGCACCAGGA |
| ScFv | | AAGGGACTGGAATGGGTGTCCGCCATTAGCGGCTCCGGCGGT |
| domain | | AGCACCTACTATGCCGACTCAGTGAAGGGAAGGTTCACTATC |
| | | TCCCGCGACAACAGCAAGAACACCCTGTACCTCCAAATGAAC |
| | | TCTCTGCGGGCCGAGGATACCGCGGTGTACTATTGCGCCAAG |
| | | ATGGGTTGGTCCAGCGGATACTTGGGAGCCTTCGACATTTGG |
| | | GGACAGGGCACTACTGTGACCGTGTCCTCCGGGGGTGGCGGA |
| | | TCGGGAGGCGGCGGCTCGGGTGGAGGGGGTTCCGAAATCGTG |
| | | TTGACCCAGTCACCGGGAACCCTCTCGCTGTCCCCGGGAGAA |
| | | CGGGCTACACTGTCATGTAGAGCGTCCCAGTCCGTGGCTTCCT |
| | | CGTTCCTGGCCTGGTACCAGCAGAAGCCGGGACAGGCACCCC |
| | | GCCTGCTCATCTACGGAGCCAGCGGCCGGGCGACCGGCATCC |
| | | CTGACCGCTTCTCCGGTTCCGGCTCGGGCACCGACTTTACTCT |
| | | GACCATTAGCAGGCTTGAGCCCGAGGATTTTGCCGTGTACTA |
| | | CTGCCAACACTACGGGGGGAGCCCTCGCCTGACCTTCGGAGG |
| | | CGGAACTAAGGTCGATATCAAAA |
| BCMA_EBB- | 1613 | EVQLVESGGGLVQPGGSLRLSCAASGFTFSSYAMSWVRQAPGK |
| C1978- | | GLEWVSAISGSGGSTYYADSVKGRFTISRDNSKNTLYLQMNSLR |
| G4-aa | | AEDTAVYYCAKMGWSSGYLGAFDIWGQGTTVTVSS |
| VH | | |
| BCMA_EBB- | 1614 | EIVLTQSPGTLSLSPGERATLSCRASQSVASSFLAWYQQKPGQAP |
| C1978- | | RLLIYGASGRATGIPDRFSGSGSGTDFTLTISRLEPEDFAVYYCQH |
| G4-aa | | YGGSPRLTFGGGTKVDIK |
| VL | | |
| BCMA_EBB- | 1615 | MALPVTALLLPLALLLHAARPEVQLVESGGGLVQPGGSLRLSCA |
| C1978- | | ASGFTFSSYAMSWVRQAPGKGLEWVSAISGSGGSTYYADSVKG |
| G4-aa | | RFTISRDNSKNTLYLQMNSLRAEDTAVYYCAKMGWSSGYLGAF |
| Full CART | | DIWGQGTTVTVSSGGGGSGGGGSGGGGSEIVLTQSPGTLSLSPGE |
| | | RATLSCRASQSVASSFLAWYQQKPGQAPRLLIYGASGRATGIPD |
| | | RFSGSGSGTDFTLTISRLEPEDFAVYYCQHYGGSPRLTFGGGTKV |
| | | DIKTTTPAPRPPTPAPTIASQPLSLRPEACRPAAGGAVHTRGLDFA |
| | | CDIYIWAPLAGTCGVLLLSLVITLYCKRGRKKLLYIFKQPFMRPV |
| | | QTTQEEDGCSCRFPEEEEGGCELRVKFSRSADAPAYKQGQNQLY |
| | | NELNLGRREEYDVLDKRRGRDPEMGGKPRRKNPQEGLYNELQK |
| | | DKMAEAYSEIGMKGERRRGKGHDGLYQGLSTATKDTYDALHM |
| | | QALPPR |
| BCMA_EBB- | 1616 | ATGGCCCTCCCTGTCACCGCCCTGCTGCTTCCGCTGGCTCTTC |
| C1978- | | TGCTCCACGCCGCTCGGCCCGAAGTCCAACTGGTGGAGTCCG |
| G4-nt | | GGGGAGGGCTCGTGCAGCCCGGAGGCAGCCTTCGGCTGTCGT |
| Full CART | | GCGCCGCCTCCGGGTTCACGTTCTCATCCTACGCGATGTCGTG |
| | | GGTCAGACAGGCACCAGGAAAGGGACTGGAATGGGTGTCCG |
| | | CCATTAGCGGCTCCGGCGGTAGCACCTACTATGCCGACTCAG |
| | | TGAAGGGAAGGTTCACTATCTCCCGCGACAACAGCAAGAACA |
| | | CCCTGTACCTCCAAATGAACTCTCTGCGGGCCGAGGATACCG |
| | | CGGTGTACTATTGCGCCAAGATGGGTTGGTCCAGCGGATACT |
| | | TGGGAGCCTTCGACATTTGGGGACAGGGCACTACTGTGACCG |
| | | TGTCCTCCGGGGGTGGCGGATCGGGAGGCGGCGGCTCGGGTG |
| | | GAGGGGGTTCCGAAATCGTGTTGACCCAGTCACCGGGAACCC |
| | | TCTCGCTGTCCCCGGGAGAACGGGCTACACTGTCATGTAGAG |
| | | CGTCCCAGTCCGTGGCTTCCTCGTTCCTGGCCTGGTACCAGCA |
| | | GAAGCCGGGACAGGCACCCCGCCTGCTCATCTACGGAGCCAG |
| | | CGGCCGGGCGACCGGCATCCCTGACCGCTTCTCCGGTTCCGG |
| | | CTCGGGCACCGACTTTACTCTGACCATTAGCAGGCTTGAGCCC |
| | | GAGGATTTTGCCGTGTACTACTGCCAACACTACGGGGGGAGC |
| | | CCTCGCCTGACCTTCGGAGGCGGAACTAAGGTCGATATCAAA |
| | | ACCACTACCCCAGCACCGAGGCCACCCACCCCGGCTCCTACC |
| | | ATCGCCTCCCAGCCTCTGTCCCTGCGTCCGGAGGCATGTAGAC |
| | | CCGCAGCTGGTGGGGCCGTGCATACCCGGGGTCTTGACTTCG |
| | | CCTGCGATATCTACATTTGGGCCCCTCTGGCTGGTACTTGCGG |
| | | GGTCCTGCTGCTTTCACTCGTGATCACTCTTTACTGTAAGCGC |
| | | GGTCGGAAGAAGCTGCTGTACATCTTTAAGCAACCCTTCATG |
| | | AGGCCTGTGCAGACTACTCAAGAGGAGGACGGCTGTTCATGC |
| | | CGGTTCCCAGAGGAGGAGGAAGGCGGCTGCGAACTGCGCGT |
| | | GAAATTCAGCCGCAGCGCAGATGCTCCAGCCTACAAGCAGGG |
| | | GCAGAACCAGCTCTACAACGAACTCAATCTTGGTCGGAGAGA |
| | | GGAGTACGACGTGCTGGACAAGCGGAGAGGACGGGACCCAG |
| | | AAATGGGCGGGAAGCCGCGCAGAAAGAATCCCCAAGAGGGC |
| | | CTGTACAACGAGCTCCAAAAGGATAAGATGGCAGAAGCCTAT |
| | | AGCGAGATTGGTATGAAAGGGGAACGCAGAAGAGGCAAAGG |
| | | CCACGACGGACTGTACCAGGGACTCAGCACCGCCACCAAGGA |
| | | CACCTATGACGCTCTTCACATGCAGGCCCTGCCGCCTCGG |

TABLE 4E
Additional exemplary BCMA CAR sequences

| | | SEQ |
|---|---|---|
| Name | Sequence | ID NO: |
| A7D12.2 | QIQLVQSGPDLKKPGETVKLSCKASGYTFTNFGMNWVKQAPGK | 1617 |
| VH | GFKWMAWINTYTGESYFADDFKGRFAFSVETSATTAYLQINNLK | |
| | TEDTATYFCARGEIYYGYDGGFAYWGQGTLVTVSA | |
| A7D12.2 | DVVMTQSHRFMSTSVGDRVSITCRASQDVNTAVSWYQQKPGQS | 1618 |
| VL | PKLLIFSASYRYTGVPDRFTGSGSGADFTLTISSVQAEDLAVYYCQ | |
| | QHYSTPWTFGGGTKLDIK | |
| A7D12.2 | QIQLVQSGPDLKKPGETVKLSCKASGYTFTNFGMNWVKQAPGK | 1619 |
| scFv | GFKWMAWINTYTGESYFADDFKGRFAFSVETSATTAYLQINNLK | |
| domain | TEDTATYFCARGEIYYGYDGGFAYWGQGTLVTVSAGGGGSGGG | |
| | GSGGGGSDVVMTQSHRFMSTSVGDRVSITCRASQDVNTAVSWY | |
| | QQKPGQSPKLLIFSASYRYTGVPDRFTGSGSGADFTLTISSVQAED | |
| | LAVYYCQQHYSTPWTFGGGTKLDIK | |
| A7D12.2 | QIQLVQSGPDLKKPGETVKLSCKASGYTFTNFGMNWVKQAPGK | 1620 |
| Full | GFKWMAWINTYTGESYFADDFKGRFAFSVETSATTAYLQINNLK | |
| CART | TEDTATYFCARGEIYYGYDGGFAYWGQGTLVTVSAGGGGSGGG | |
| | GSGGGGSDVVMTQSHRFMSTSVGDRVSITCRASQDVNTAVSWY | |
| | QQKPGQSPKLLIFSASYRYTGVPDRFTGSGSGADFTLTISSVQAED | |
| | LAVYYCQQHYSTPWTFGGGTKLDIKTTTPAPRPPTPAPTIASQPLS | |
| | LRPEACRPAAGGAVHTRGLDFACDIYIWAPLAGTCGVLLLSLVIT | |
| | LYCKRGRKKLLYIFKQPFMRPVQTTQEEDGCSCRFPEEEEGGCEL | |
| | RVKFSRSADAPAYKQGQNQLYNELNLGRREEYDVLDKRRGRDP | |
| | EMGGKPRRKNPQEGLYNELQKDKMAEAYSEIGMKGERRRGKG | |
| | HDGLYQGLSTATKDTYDALHMQALPPR | |
| C11D5.3 | QIQLVQSGPELKKPGETVKISCKASGYTFTDYSINWVKRAPGKGL | 1621 |
| VH | KWMGWINTETREPAYAYDFRGRFAFSLETSASTAYLQINNLKYE | |
| | DTATYFCALDYSYAMDYWGQGTSVTVSS | |
| C11D5.3 | DIVLTQSPASLAMSLGKRATISCRASESVSVIGAHLIHWYQQKPG | 1622 |
| VL | QPPKLLIYLASNLETGVPARFSGSGSGTDFTLTIDPVEEDDVAIYS | |
| | CLQSRIFPRTFGGGTKLEIK | |
| C11D5.3 | QIQLVQSGPELKKPGETVKISCKASGYTFTDYSINWVKRAPGKGL | 1623 |
| scFv | KWMGWINTETREPAYAYDFRGRFAFSLETSASTAYLQINNLKYE | |
| domain | DTATYFCALDYSYAMDYWGQGTSVTVSSGGGGSGGGGSGGGG | |
| | SQIQLVQSGPELKKPGETVKISCKASGYTFTDYSINWVKRAPGKG | |
| | LKWMGWINTETREPAYAYDFRGRFAFSLETSASTAYLQINNLKY | |
| | EDTATYFCALDYSYAMDYWGQGTSVTVSS | |
| C11D5.3 | QIQLVQSGPELKKPGETVKISCKASGYTFTDYSINWVKRAPGKGL | 1624 |
| Full | KWMGWINTETREPAYAYDFRGRFAFSLETSASTAYLQINNLKYE | |
| CART | DTATYFCALDYSYAMDYWGQGTSVTVSSGGGGSGGGGSGGGG | |
| | SQIQLVQSGPELKKPGETVKISCKASGYTFTDYSINWVKRAPGKG | |
| | LKWMGWINTETREPAYAYDFRGRFAFSLETSASTAYLQINNLKY | |
| | EDTATYFCALDYSYAMDYWGQGTSVTVSSTTTPAPRPPTPAPTIA | |
| | SQPLSLRPEACRPAAGGAVHTRGLDFACDIYIWAPLAGTCGVLLL | |
| | SLVITLYCKRGRKKLLYIFKQPFMRPVQTTQEEDGCSCRFPEEEEG | |
| | GCELRVKFSRSADAPAYKQGQNQLYNELNLGRREEYDVLDKRR | |
| | GRDPEMGGKPRRKNPQEGLYNELQKDKMAEAYSEIGMKGERRR | |
| | GKGHDGLYQGLSTATKDTYDALHMQALPPR | |
| C12A3.2 | QIQLVQSGPELKKPGETVKISCKASGYTFRHYSMNWVKQAPGKG | 1625 |
| VH | LKWMGRINTESGVPIYADDFKGRFAFSVETSASTAYLVINNLKDE | |
| | DTASYFCSNDYLYSLDFWGQGTALTVSS | |
| C12A3.2 | DIVLTQSPPSLAMSLGKRATISCRASESVTILGSHLIYWYQQKPGQ | 1626 |
| VL | PPTLLIQLASNVQTGVPARFSGSGSRTDFTLTIDPVEEDDVAVYYC | |
| | LQSRTIPRTFGGGTKLEIK | |
| C12A3.2 | QIQLVQSGPELKKPGETVKISCKASGYTFRHYSMNWVKQAPGKG | 1627 |
| scFv | LKWMGRINTESGVPIYADDFKGRFAFSVETSASTAYLVINNLKDE | |
| domain | DTASYFCSNDYLYSLDFWGQGTALTVSSGGGGSGGGGSGGGGS | |
| | DIVLTQSPPSLAMSLGKRATISCRASESVTILGSHLIYWYQQKPGQ | |
| | PPTLLIQLASNVQTGVPARFSGSGSRTDFTLTIDPVEEDDVAVYYC | |
| | LQSRTIPRTFGGGTKLEIK | |
| C12A3.2 | QIQLVQSGPELKKPGETVKISCKASGYTFRHYSMNWVKQAPGKG | 1628 |
| Full | LKWMGRINTESGVPIYADDFKGRFAFSVETSASTAYLVINNLKDE | |
| CART | DTASYFCSNDYLYSLDFWGQGTALTVSSGGGGSGGGGSGGGGS | |
| | DIVLTQSPPSLAMSLGKRATISCRASESVTILGSHLIYWYQQKPGQ | |
| | PPTLLIQLASNVQTGVPARFSGSGSRTDFTLTIDPVEEDDVAVYYC | |
| | LQSRTIPRTFGGGTKLEIKTTTPAPRPPTPAPTIASQPLSLRPEACRP | |
| | AAGGAVHTRGLDFACDIYIWAPLAGTCGVLLLSLVITLYCKRGR | |
| | KKLLYIFKQPFMRPVQTTQEEDGCSCRFPEEEEGGCELRVKFSRS | |
| | ADAPAYKQGQNQLYNELNLGRREEYDVLDKRRGRDPEMGGKP | |
| | RRKNPQEGLYNELQKDKMAEAYSEIGMKGERRRGKGHDGLYQ | |
| | GLSTATKDTYDALHMQALPPR | |
| C13F12.1 | QIQLVQSGPELKKPGETVKISCKASGYTFTHYSMNWVKQAPGKG | 1629 |
| VH | LKWMGRINTETGEPLYADDFKGRFAFSLETSASTAYLVINNLKNE | |
| | DTATFFCSNDYLYSCDYWGQGTTLTVSS | |
| C13F12.1 | DIVLTQSPPSLAMSLGKRATISCRASESVTILGSHLIYWYQQKPGQ | 1630 |
| VL | PPTLLIQLASNVQTGVPARFSGSGSRTDFTLTIDPVEEDDVAVYYC | |
| | LQSRTIPRTFGGGTKLEIK | |
| C13F12.1 | QIQLVQSGPELKKPGETVKISCKASGYTFTHYSMNWVKQAPGKG | 1631 |
| scFv | LKWMGRINTETGEPLYADDFKGRFAFSLETSASTAYLVINNLKNE | |
| domain | DTATFFCSNDYLYSCDYWGQGTTLTVSSGGGGSGGGGSGGGGS | |
| | DIVLTQSPPSLAMSLGKRATISCRASESVTILGSHLIYWYQQKPGQ | |
| | PPTLLIQLASNVQTGVPARFSGSGSRTDFTLTIDPVEEDDVAVYYC | |
| | LQSRTIPRTFGGGTKLEIK | |
| C13F12.1 | QIQLVQSGPELKKPGETVKISCKASGYTFTHYSMNWVKQAPGKG | 1632 |
| Full | LKWMGRINTETGEPLYADDFKGRFAFSLETSASTAYLVINNLKNE | |
| CART | DTATFFCSNDYLYSCDYWGQGTTLTVSSGGGGSGGGGSGGGGS | |
| | DIVLTQSPPSLAMSLGKRATISCRASESVTILGSHLIYWYQQKPGQ | |
| | PPTLLIQLASNVQTGVPARFSGSGSRTDFTLTIDPVEEDDVAVYYC | |
| | LQSRTIPRTFGGGTKLEIKTTTPAPRPPTPAPTIASQPLSLRPEACRP | |
| | AAGGAVHTRGLDFACDIYIWAPLAGTCGVLLLSLVITLYCKRGR | |
| | KKLLYIFKQPFMRPVQTTQEEDGCSCRFPEEEEGGCELRVKFSRS | |
| | ADAPAYKQGQNQLYNELNLGRREEYDVLDKRRGRDPEMGGKP | |
| | RRKNPQEGLYNELQKDKMAEAYSEIGMKGERRRGKGHDGLYQ | |
| | GLSTATKDTYDALHMQALPPR | |

TABLE 4F
Amino acid sequences of exemplary BCMA binding domains

| | | |
|---|---|---|
| ER26 | | |
| SEQ ID NO: | J6M0 VH | QVQLVQSGAEVKKPGSSVKVSCKASGGTFSNYWMHW |
| 1635 | | VRQAPGQGLEWMGATYRGHSDTYYNQKFKGRVTITA |
| | | DKSTSTAYMELSSLRSEDTAVYYCARGAIYDGYDVLD |
| | | NWGQGTLVTVSS |
| SEQ ID NO: | J6M0 VL | DIQMTQSPSSLSASVGDRVTITCSASQDISNYLNWYQQ |
| 1636 | | KPGKAPKLLIYYTSNLHSGVPSRFSGSGSGTDFTLTISSL |
| | | QPEDFATYYCQQYRKLPWTFGQGTKLEIKR |
| SEQ ID NO: | Anti-BCMA | QVQLVQSGAEVKKPGSSVKVSCKASGGTFSNYWMHW |
| 1637 | heavy chain | VRQAPGQGLEWMGATYRGHSDTYYNQKFKGRVTITA |
| | of ER26 | DKSTSTAYMELSSLRSEDTAVYYCARGAIYNGYDVLD |
| | | NWGQGTLVTVSSASTKGPSVFPLAPSSKSTSGGTAALG |
| | | CLVKDYFPEPVTVSWNSGALTSGVHTFPAVLQSSGLYS |
| | | LSSVVTVPSSSLGTQTYICNVNHKPSNTKVDKRVEPKSC |
| | | DKTHTCPPCPAPELLGGPSVFLFPPKPKDTLMISRTPEVT |
| | | CVVVAVSHEDPEVKFNWYVDGVEVHNAKTKPREEQY |
| | | NSTYRVVSVLTVLHQDWLNGKEYKCKVSNKALAAPIE |
| | | KTISKAKGQPREPQVCTLPPSREEMTKNQVSLSCAVKG |
| | | FYPSDIAVEWESNGQPENNYKTTPPVLDSDGSFFLVSKL |
| | | TVDKSRWQQGNVFSCSVMHEALHNHYTQKSLSLSPGX, |
| | | wherein X is K or absent |
| SEQ ID NO: | Anti-BCMA | DIQMTQSPSSLSASVGDRVTITCSASQDISNYLNWYQQ |
| 1638 | light chain of | KPGKAPKLLIYYTSNLHSGVPSRFSGSGSGTDFTLTISSL |
| | ER26 | QPEDFATYYCQQYRKLPWTFGQGTKLEIKRTVAAPSVF |
| | | IFPPSDEQLKSGTASVVCLLNNFYPREAKVQWKVDNAL |
| | | QSGNSQESVTEQDSKDSTYSLSSTLTLSKADYEKHKVY |
| | | ACEVTHQGLSSPVTKSFNRGEC |
| BQ76 | | |
| SEQ ID NO: | 17A5 VH | EVQLLESGGGLVQPGGSLRLSCAASGFTFSSYAMSWVR |
| 1639 | | QAPGKGLEWVSAISGSGGSTYYADSVKGRFTISRDNSK |
| | | NTLYLQMNSLRAEDTAVYYCAKVAPYFAPFDYWGQG |
| | | TLVTVSS |
| SEQ ID NO: | 17A5 VL | EIVLTQSPGTLSLSPGERATLSCRASQSVSSSYLAWYQQ |
| 1640 | | KPGQAPRLLIYGASSRATGIPDRFSGSGSGTDFTLTISRL |
| | | EPEDFAVYYCQQYGNPPLYTFGQGTKVEIK |
| SEQ ID NO: | Anti-BCMA | EVQLLESGGGLVQPGGSLRLSCAASGFTFSSYAMSWVR |
| 1641 | heavy chain | QAPGKGLEWVSAISGSGGSTYYADSVKGRFTISRDNSK |
| | of BQ76 | NTLYLQMNSLRAEDTAVYYCAKVAPYFAPFDYWGQG |
| | | TLVTVSSASTKGPSVFPLAPSSKSTSGGTAALGCLVKDY |
| | | FPEPVTVSWNSGALTSGVHTFPAVLQSSGLYSLSSVVT |
| | | VPSSSLGTQTYICNVNHKPSNTKVDKRVEPKSCDKTHT |
| | | CPPCPAPELLGGPSVFLFPPKPKDTLMISRTPEVTCVVV |
| | | DVSHEDPEVKFNWYVDGVEVHNAKTKPREEQYASTY |
| | | RVVSVLTVLHQDWLNGKEYKCKVSNKALPAPIEKTISK |
| | | AKGQPREPQVCTLPPSREEMTKNQVSLSCAVKGFYPSD |
| | | IAVEWESNGQPENNYKTTPPVLDSDGSFFLVSKLTVDK |
| | | SRWQQGNVFSCSVMHEALHNHYTQKSLSLSPGX, |
| | | wherein X is K or absent |
| SEQ ID NO: | Anti-BCMA | EIVLTQSPGTLSLSPGERATLSCRASQSVSSSYLAWYQQ |
| 1642 | light chain of | KPGQAPRLLIYGASSRATGIPDRFSGSGSGTDFTLTISRL |
| | BQ76 | EPEDFAVYYCQQYGNPPLYTFGQGTKVEIKRTVAAPSV |
| | | FIFPPSDEQLKSGTASVVCLLNNFYPREAKVQWKVDNA |
| | | LQSGNSQESVTEQDSKDSTYSLSSTLTLSKADYEKHKV |
| | | YACEVTHQGLSSPVTKSFNRGEC |
| BU76 | | |
| SEQ ID NO: | C11D5 VH | QIQLVQSGPELKKPGETVKISCKASGYTFTDYSINWVKR |
| 1643 | | APGKGLKWMGWINTETREPAYAYDFRGRFAFSLETSA |
| | | STAYLQINNLKYEDTATYFCALDYSYAMDYWGQGTSV |
| | | TVSS |
| SEQ ID NO: | C11D5 VL | DIVLTQSPPSLAMSLGKRATISCRASESVTILGSHLIHWY |
| 1644 | | QQKPGQPPTLLIQLASNVQTGVPARFSGSGSRTDFTLTI |
| | | DPVEEDDVAVYYCLQSRTIPRTFGGGTKLEIK |
| SEQ ID NO: | Anti-BCMA | QIQLVQSGPELKKPGETVKISCKASGYTFTDYSINWVKR |
| 1645 | heavy chain | APGKGLKWMGWINTETREPAYAYDFRGRFAFSLETSA |
| | of BU76 | STAYLQINNLKYEDTATYFCALDYSYAMDYWGQGTSV |
| | | TVSSASTKGPSVFPLAPSSKSTSGGTAALGCLVKDYFPE |
| | | PVTVSWNSGALTSGVHTFPAVLQSSGLYSLSSVVTVPSS |
| | | SLGTQTYICNVNHKPSNTKVDKRVEPKSCDKTHTCPPC |
| | | PAPELLGGPSVFLFPPKPKDTLMISRTPEVTCVVVDVSH |
| | | EDPEVKFNWYVDGVEVHNAKTKPREEQYASTYRVVS |
| | | VLTVLHQDWLNGKEYKCKVSNKALPAPIEKTISKAKG |
| | | QPREPQVCTLPPSREEMTKNQVSLSCAVKGFYPSDIAVE |
| | | WESNGQPENNYKTTPPVLDSDGSFFLVSKLTVDKSRW |
| | | QQGNVFSCSVMHEALHNHYTQKSLSLSPGX, wherein X |
| | | is K or absent |
| SEQ ID NO: | Anti-BCMA | DIVLTQSPASLAMSLGKRATISCRASESVSVIGAHLIHW |
| 1646 | light chain of | YQQKPGQPPKLLIYLASNLETGVPARFSGSGSGTDFTLT |
| | BU76 | IDPVEEDDVAIYSCLQSRlFPRTFGGGTKLEIKRTVAAPS |
| | | VFIFPPSDEQLKSGTASVVCLLNNFYPREAKVQWKVDN |
| | | ALQSGNSQESVTEQDSKDSTYSLSSTLTLSKADYEKHK |
| | | VYACEVTHQGLSSPVTKSFNRGEC |
| EE11 | | |
| SEQ ID NO: | 83A10 VH | EVQLLESGGGLVQPGGSLRLSCAASGFTFSSYAMSWVR |
| 1647 | | QAPGKGLEWVSAISGSGGSTYYADSVKGRFTISRDNSK |
| | | NTLYLQMNSLRAEDTAVYYCAKVLGWFDYWGQGTLV |
| | | TVSS |
| SEQ ID NO: | 83A10 VL | EIVLTQSPGTLSLSPGERATLSCRASQSVSSSYLAWYQQ |
| 1648 | | KPGQAPRLLIYGASSRATGIPDRFSGSGSGTDFTLTISRL |
| | | EPEDFAVYYCQQYGYPPDFTFGQGTKVEIK |
| SEQ ID NO: | Anti-BCMA | EVQLLESGGGLVQPGGSLRLSCAASGFTFSSYAMSWVR |
| 1649 | scFv-Fc of | QAPGKGLEWVSAISGSGGSTYYADSVKGRFTISRDNSK |
| | EE11 | NTLYLQMNSLRAEDTAVYYCAKVLGWFDYWGQGTLV |
| | | TVSSGGGGSGGGGSGGGGSGGGGSEIVLTQSPGTLSLSP |
| | | GERATLSCRASQSVSSSYLAWYQQKPGQAPRLLIYGAS |
| | | SRATGIPDRFSGSGSGTDFTLTISRLEPEDFAVYYCQQY |
| | | GYPPDFTFGQGTKVEIKGGGGSDKTHTCPPCPAPELLG |
| | | GPSVFLFPPKPKDTLMISRTPEVTCVVVAVSHEDPEVKF |
| | | NWYVDGVEVHNAKTKPREEQYNSTYRVVSVLTVLHQ |
| | | DWLNGKEYKCKVSNKALAAPIEKTISKAKGQPREPQV |
| | | YTLPPCRDELTKNQVSLWCLVKGFYPSDIAVEWESNGQ |
| | | PENNYKTTPPVLDSDGSFFLYSKLTVDKSRWQQGNVFS |
| | | CSVMHEALHNHYTQKSLSLSPGX, wherein X is K or |
| | | absent |
| EM90 | | |
| SEQ ID NO: | Comment | QAVVTQEPSLTVSPGGTVTLTCGSSTGAVTTSNYANW |
| 1650 | light chain of | VQEKPGQAFRGLIGGTNKRAPGTPARFSGSLLGGKAAL |
| | EM90 | TLSGAQPEDEAEYYCALWYSNLWVFGGGTKLTVLGQP |
| | | KAAPSVTLFPPSSEELQANKATLVCLISDFYPGAVTVA |
| | | WKADSSPVKAGVETTTPSKQSNNKYAASSYLSLTPEQ |
| | | WKSHRSYSCQVTHEGSTVEKTVAPTECS |
| SEQ ID NO: | Anti-BCMA | EVQLVESGGGLVKPGGSLRLSCAASGFTFSNSGMIWVR |
| 1651 | heavy chain | QAPGKGLEWVGHIRSKTDGGTTDYAAPVKGRFTISRD |
| | of EM90 | DSKNTLYLQMNSLKTEDTAVYYCTTGGSGSFDYWGQ |
| | | GTLVTVSSASTKGPSVFPLAPSSKSTSGGTAALGCLVKD |
| | | YFPEPVTVSWNSGALTSGVHTFPAVLQSSGLYSLSSVV |
| | | TVPSSSLGTQTYICNVNHKPSNTKVDKRVEPKSCDKTH |
| | | TCPPCPAPEAAGGPSVFLFPPKPKDTLMISRTPEVTCVV |
| | | VDVSHEDPEVKFNWYVDGVEVHNAKTKPREEQYNST |
| | | YRVVSVLTVLHQDWLNGKEYKCKVSNKALPAPIEKTIS |
| | | KAKGQPREPQVCTLPPSREEMTKNQVSLSCAVKGFYPS |
| | | DIAVEWESNGQPENNYKTTPPVLDSDGSFFLVSKLTVD |
| | | KSRWQQGNVFSCSVMHEALHNHYTQKSLSLSPGX, |
| | | wherein X is K or absent |

TABLE 4G
Heavy Chain Variable Domain CDRs according to the
Kabat numbering scheme (Kabat et al. (1991), “Sequences
of Proteins of Immunological Interest,” 5th Ed.
Public Health Service, National Institutes of Health,
Bethesda, MD)

| Candidate | HCDR1 | ID | HCDR2 | ID | HCDR3 | ID |
|---|---|---|---|---|---|---|
| 139109 | NHGMS | 1652 | GIVYSGSTYYAA | 1701 | HGGESDV | 1741 |
| | | | SVKG | | | |
| 139103 | NYAMS | 1653 | GISRSGENTYYA | 1702 | SPAHYYGGMDV | 1742 |
| | | | DSVKG | | | |
| 139105 | DYAM | 1654 | GISWNSGSIGYA | 1703 | HSFLAY | 1743 |
| | | | DSVKG | | | |
| 139111 | NHGMS | 1655 | GIVYSGSTYYAA | 1704 | HGGESDV | 1744 |
| | | | SVKG | | | |
| 139100 | NFGIN | 1656 | WINPKNNNTNY | 1705 | GPYYYQSYMDV | 1745 |
| | | | AQKFQG | | | |
| 139101 | SDAMT | 1657 | VISGSGGTTYYA | 1706 | LDSSGYYYARGP | 1746 |
| | | | DSVKG | | RY | |
| 139102 | NYGIT | 1658 | WISAYNGNTNY | 1707 | GPYYYYMDV | 1747 |
| | | | AQKFQG | | | |
| 139104 | NHGMS | 1659 | GIVYSGSTYYAA | 1708 | HGGESDV | 1748 |
| | | | SVKG | | | |
| 139106 | NHGMS | 1670 | GIVYSGSTYYAA | 1709 | HGGESDV | 1749 |
| | | | SVKG | | | |
| 139107 | NHGMS | 1671 | GIVYSGSTYYAA | 1710 | HGGESDV | 1750 |
| | | | SVKG | | | |
| 139108 | DYYMS | 1672 | YISSSGSTIYYAD | 1711 | ESGDGMDV | 1751 |
| | | | SVKG | | | |
| 139110 | DYYMS | 1672 | YISSSGNTIYYAD | 1712 | STMVREDY | 1752 |
| | | | SVKG | | | |
| 139112 | NHGMS | 1673 | GIVYSGSTYYAA | 1713 | HGGESDV | 1753 |
| | | | SVKG | | | |
| 139113 | NHGMS | 1674 | GIVYSGSTYYAA | 1714 | HGGESDV | 1754 |
| | | | SVKG | | | |
| 139114 | NHGMS | 1675 | GIVYSGSTYYAA | 1715 | HGGESDV | 1755 |
| | | | SVKG | | | |
| 149362 | SSYYY | 1676 | SIYYSGSAYYNP | 1716 | HWQEWPDAFDI | 1756 |
| | WG | | SLKS | | | |
| 149363 | TSGMC | 1677 | RIDWDEDKFYST | 1717 | SGAGGTSATAFDI | 1757 |
| | VS | | SLKT | | | |
| 149364 | SYSMN | 1678 | SISSSSSYIYYAD | 1718 | TIAAVYAFDI | 1758 |
| | | | SVKG | | | |
| 149365 | DYYMS | 1679 | YISSSGSTIYYAD | 1719 | DLRGAFDI | 1759 |
| | | | SVKG | | | |
| 149366 | SHYIH | 1680 | MINPSGGVTAYS | 1720 | EGSGSGWYFDF | 1760 |
| | | | QTLQG | | | |
| 149367 | SGGYY | 1681 | YIYYSGSTYYNP | 1721 | AGIAARLRGAFDI | 1761 |
| | WS | | SLKS | | | |
| 149368 | SYAIS | 1682 | GIIPIFGTANYAQ | 1722 | RGGYQLLRWDV | 1762 |
| | | | KFQG | | GLLRSAFDI | |
| 149369 | SNSAA | 1683 | RTYYRSKWYSF | 1723 | SSPEGLFLYWFDP | 1763 |
| | WN | | YAISLKS | | | |
| BCMA_EBB- | SYAMS | 1684 | AISGSGGSTYYA | 1724 | VEGSGSLDY | 1764 |
| C1978- | | | DSVKG | | | |
| A4 | | | | | | |
| BCMA_EBB- | RYPMS | 1685 | GISDSGVSTYYA | 1725 | RAGSEASDI | 1765 |
| C1978- | | | DSAKG | | | |
| G1 | | | | | | |
| BCMA_EBB- | SYAMS | 1686 | AISGSGGSTYYA | 1726 | ATYKRELRYYY | 1766 |
| C1979- | | | DSVKG | | GMDV | |
| C1 | | | | | | |
| BCMA_EBB- | SYAMS | 1687 | AISGSGGSTYYA | 1727 | ATYKRELRYYY | 1767 |
| C1978- | | | DSVKG | | GMDV | |
| C7 | | | | | | |
| BCMA_EBB- | DYAMH | 1688 | GISWNSGSIGYA | 1728 | VGKAVPDV | 1768 |
| C1978- | | | DSVKG | | | |
| D10 | | | | | | |
| BCMA_EBB- | DYAMH | 1689 | SINWKGNSLAY | 1729 | HQGVAYYNYAM | 1769 |
| C1979- | | | GDSVKG | | DV | |
| C12 | | | | | | |
| BCMA_EBB- | SYAMS | 1690 | AISGSGGSTYYA | 1730 | VVRDGMDV | 1770 |
| C1980- | | | DSVKG | | | |
| G4 | | | | | | |
| BCMA_EBB- | SYAMS | 1691 | AISGSGGSTYYA | 1731 | IPQTGTFDY | 1771 |
| C1980- | | | DSVKG | | | |
| D2 | | | | | | |
| BCMA_EBB- | SYAMS | 1692 | AISGSGGSTYYA | 1732 | ANYKRELRYYY | 1772 |
| C1978- | | | DSVKG | | GMDV | |
| A10 | | | | | | |
| BCMA_EBB- | SYAMS | 1693 | AISGSGGSTYYA | 1733 | ALVGATGAFDI | 1773 |
| C1978- | | | DSVKG | | | |
| D4 | | | | | | |
| BCMA_EBB- | SYAMS | 1694 | AISGSGGSTYYA | 1734 | WFGEGFDP | 1774 |
| C1980- | | | DSVKG | | | |
| A2 | | | | | | |
| BCMA_EBB- | SYAMS | 1695 | AISGSGGSTYYA | 1735 | VGYDSSGYYRD | 1775 |
| C1981- | | | DSVKG | | YYGMDV | |
| C3 | | | | | | |
| BCMA_EBB- | SYAMS | 1696 | AISGSGGSTYYA | 1736 | MGWSSGYLGAF | 1776 |
| C1978- | | | DSVKG | | DI | |
| G4 | | | | | | |
| A7D12.2 | NFGMN | 1697 | WINTYTGESYFA | 1737 | GEIYYGYDGGFAY | 1777 |
| | | | DDFKG | | | |
| C11D5.3 | DYSIN | 1698 | WINTETREPAYA | 1738 | DYSYAMDY | 1778 |
| | | | YDFRG | | | |
| C12A3.2 | HYSMN | 1699 | RINTESGVPIYAD | 1739 | DYLYSLDF | 1779 |
| | | | DFKG | | | |
| C13F12.1 | HYSMN | 1700 | RINTETGEPLYA | 1740 | DYLYSCDY | 1800 |
| | | | DDFKG | | | |

TABLE 4H
Light Chain Variable Domain CDRs according to the
Kabat numbering scheme (Kabat et al. (1991), “Sequences
of Proteins of Immunological Interest,” 5th Ed.
Public Health Service, National Institutes of Health,
Bethesda, MD).

| Candidate | LCDR1 | ID | LCDR2 | ID | LCDR3 | ID |
|---|---|---|---|---|---|---|
| 139109 | RASQSISSYLN | 2514 | AASSLQS | 2554 | QQSYSTPYT | 2594 |
| 139103 | RASQSISSSFLA | 2504 | GASRRAT | 2544 | QQYHSSPS | 2584 |
| | | | | | WT | |
| 139105 | RSSQSLLHSNG | 2505 | LGSNRAS | 2545 | MQALQTPYT | 2585 |
| | YNYLD | | | | | |
| 139111 | KSSQSLLRNDG | 2506 | EVSNRFS | 2546 | MQNIQFPS | 2586 |
| | KTPLY | | | | | |
| 139100 | RSSQSLLHSNG | 2507 | LGSKRAS | 2547 | MQALQTPYT | 2587 |
| | YNYLN | | | | | |
| 139101 | RASQSISSYLN | 2508 | GASTLAS | 2548 | QQSYKRAS | 2588 |
| 139102 | RSSQSLLYSNG | 2509 | LGSNRAS | 2549 | MQGRQFPYS | 2589 |
| | YNYVD | | | | | |
| 139104 | RASQSVSSNLA | 2510 | GASTRAS | 2550 | QQYGSSLT | 2590 |
| 139106 | RASQSVSSKLA | 2511 | GASIRAT | 2551 | QQYGSSSWT | 2591 |
| 139107 | RASQSVGSTNLA | 2512 | DASNRAT | 2552 | QQYGSSPP | 2592 |
| | | | | | WT | |
| 139108 | RASQSISSYLN | 2513 | AASSLQS | 2553 | QQSYTLA | 2593 |
| 139110 | KSSESLVHNSG | 2515 | EVSNRDS | 2555 | MQGTHWP | 2595 |
| | KTYLN | | | | GT | |
| 139112 | QASEDINKFLN | 2516 | DASTLQT | 2556 | QQYESLPLT | 2596 |
| 139113 | RASQSVGSNLA | 2517 | GASTRAT | 2557 | QQYNDWL | 2597 |
| | | | | | PVT | |
| 139114 | RASQSIGSSSLA | 2518 | GASSRAS | 2558 | QQYAGSPP | 2598 |
| | | | | | FT | |
| 149362 | KASQDIDDAMN | 2519 | SATSPVP | 2559 | LQHDNFPLT | 2599 |
| 149363 | RASQDIYNNLA | 2520 | AANKSQS | 2560 | QHYYRFPYS | 2600 |
| 149364 | RSSQSLLHSNG | 2521 | LGSNRAS | 2561 | MQALQTPYT | 2601 |
| | YNYLD | | | | | |
| 149365 | GGNNIGTKSVH | 2522 | DDSVRPS | 2562 | QVWDSDSE | 2602 |
| | | | | | HVV | |
| 149366 | SGDGLSKKYVS | 2523 | RDKERPS | 2563 | QAWDDTT | 2603 |
| | | | | | VV | |
| 149367 | RASQGIRNWLA | 2524 | AASNLQS | 2564 | QKYNSAPFT | 2604 |
| 149368 | GGNNIGSKSVH | 2525 | GKNNRPS | 2565 | SSRDSSGD | 2605 |
| | | | | | HLRV | |
| 149369 | QGDSLGNYYAT | 2526 | GTNNRPS | 2566 | NSRDSSGH | 2606 |
| | | | | | HLL | |
| BCMA_EBB- | RASQSVSSAYLA | 2527 | GASTRAT | 2567 | QHYGSSFN | 2607 |
| C1978-A4 | | | | | GSSLFT | |
| BCMA_EBB- | RASQSVSNSLA | 2528 | DASSRAT | 2568 | QQFGTSSG | 2608 |
| C1978-G1 | | | | | LT | |
| BCMA_EBB- | RASQSVSSSFLA | 2529 | GASSRAT | 2569 | QQYHSSPS | 2609 |
| C1979-C1 | | | | | WT | |
| BCMA_EBB- | RASQSVSTTFLA | 2530 | GSSNRAT | 2570 | QQYHSSPS | 2610 |
| C1978-C7 | | | | | WT | |
| BCMA_EBB- | RASQSISSYLN | 2531 | AASSLQS | 2571 | QQSYSTPYS | 2611 |
| C1978- | | | | | | |
| D10 | | | | | | |
| BCMA_EBB- | RATQSIGSSFLA | 2532 | GASQRAT | 2572 | QHYESSPS | 2612 |
| C1979- | | | | | WT | |
| C12 | | | | | | |
| BCMA_EBB- | RASQSVSSSYLA | 2533 | GASSRAT | 2573 | QQYGSPPR | 2613 |
| C1980-G4 | | | | | FT | |
| BCMA_EBB- | RASQSVSSSYLA | 2534 | GASSRAT | 2574 | QHYGSSPS | 2614 |
| C1980-D2 | | | | | WT | |
| BCMA_EBB- | RASQRVASNYLA | 2535 | GASSRAT | 2575 | QHYDSSPS | 2615 |
| C1978- | | | | | WT | |
| A10 | | | | | | |
| BCMA_EBB- | RASQSLSSNFLA | 2536 | GASNW | 2576 | QYYGTSPM | 2616 |
| C1978-D4 | | | AT | | YT | |
| BCMA_EBB- | RSSQSLLHSNG | 2537 | LGSNRAS | 2577 | MQALQTPLT | 2617 |
| C1980-A2 | YNYLD | | | | | |
| BCMA_EBB- | RASQSVSSSYLA | 2538 | GTSSRAT | 2578 | QHYGNSPP | 2618 |
| C1981-C3 | | | | | KFT | |
| BCMA_EBB- | RASQSVASSFLA | 2539 | GASGRAT | 2579 | QHYGGSPR | 2619 |
| C1978-G4 | | | | | LT | |
| A7D12.2 | RASQDVNTAVS | 2540 | SASYRYT | 2580 | QQHYSTPWT | 2620 |
| C11D5.3 | RASESVSVIGAH | 2541 | LASNLET | 2581 | LQSRIFPRT | 2621 |
| | LIH | | | | | |
| C12A3.2 | RASESVTILGSH | 2542 | LASNVQT | 2582 | LQSRTIPRT | 2622 |
| | LIY | | | | | |
| C13F12.1 | RASESVTILGSH | 2543 | LASNVQT | 2583 | LQSRTIPRT | 2623 |
| | LIY | | | | | |

TABLE 4I
Heavy Chain Variable Domain CDRs according to a
combination of the Kabat numbering scheme (Kabat et
al. (1991), “Sequences of Proteins of Immunological
Interest,” 5th Ed. Public Health Service, National
Institutes of Health, Bethesda, MD) and the Chothia
numbering scheme (Al-Lazikani et al., (1997)
JMB 273, 927-948).

| Candidate | HCDR1 | ID | HCDR2 | ID | HCDR3 | ID |
|---|---|---|---|---|---|---|
| 139109 | GFALSNHG | 2874 | GIVYSGSTYY | 2914 | HGGESDV | 2954 |
| | MS | | AASVKG | | | |
| 139103 | GFTFSNYA | 2864 | GISRSGENTYY | 2904 | SPAHYYGGMDV | 2944 |
| | MS | | ADSVKG | | | |
| 139105 | GFTFDDYA | 2865 | GISWNSGSIGY | 2905 | HSFLAY | 2945 |
| | MH | | ADSVKG | | | |
| 139111 | GFALSNHG | 2866 | GIVYSGSTYY | 2906 | HGGESDV | 2946 |
| | MS | | AASVKG | | | |
| 139100 | GYIFDNFGIN | 2867 | WINPKNNNTN | 2907 | GPYYYQSYMDV | 2947 |
| | | | YAQKFQG | | | |
| 139101 | GFTFSSDAMT | 2868 | VISGSGGTTYY | 2908 | LDSSGYYYAR | 2948 |
| | | | ADSVKG | | GPRY | |
| 139102 | GYTFSNYGIT | 2869 | WISAYNGNTN | 2909 | GPYYYYMDV | 2949 |
| | | | YAQKFQG | | | |
| 139104 | GFALSNHG | 2870 | GIVYSGSTYY | 2910 | HGGESDV | 2950 |
| | MS | | AASVKG | | | |
| 139106 | GFALSNHG | 2871 | GIVYSGSTYY | 2911 | HGGESDV | 2951 |
| | MS | | AASVKG | | | |
| 139107 | GFALSNHG | 2872 | GIVYSGSTYY | 2912 | HGGESDV | 2952 |
| | MS | | AASVKG | | | |
| 139108 | GFTFSDYY | 2873 | YISSSGSTIYY | 2913 | ESGDGMDV | 2953 |
| | MS | | ADSVKG | | | |
| 139110 | GFTFSDYY | 2875 | YISSSGNTIYY | 2915 | STMVREDY | 2955 |
| | MS | | ADSVKG | | | |
| 139112 | GFALSNHG | 2876 | GIVYSGSTYY | 2916 | HGGESDV | 2956 |
| | MS | | AASVKG | | | |
| 139113 | GFALSNHG | 2877 | GIVYSGSTYY | 2917 | HGGESDV | 2957 |
| | MS | | AASVKG | | | |
| 139114 | GFALSNHG | 2878 | GIVYSGSTYY | 2918 | HGGESDV | 2958 |
| | MS | | AASVKG | | | |
| 149362 | GGSISSSYY | 2879 | SIYYSGSAYY | 2919 | HWQEWPDAFDI | 2959 |
| | YWG | | NPSLKS | | | |
| 149363 | GFSLRTSGM | 2880 | RIDWDEDKFY | 2920 | SGAGGTSATAF | 2960 |
| | CVS | | STSLKT | | DI | |
| 149364 | GFTFSSYSMN | 2881 | SISSSSSYIYYA | 2921 | TIAAVYAFDI | 2961 |
| | | | DSVKG | | | |
| 149365 | GFTFSDYY | 2882 | YISSSGSTIYY | 2922 | DLRGAFDI | 2962 |
| | MS | | ADSVKG | | | |
| 149366 | GYTVTSHYIH | 2883 | MINPSGGVTA | 2923 | EGSGSGWYFDF | 2963 |
| | | | YSQTLQG | | | |
| 149367 | GGSISSGGY | 2884 | YIYYSGSTYY | 2924 | AGIAARLRGAF | 2964 |
| | YWS | | NPSLKS | | DI | |
| 149368 | GGTFSSYAIS | 2885 | GIIPIFGTANY | 2925 | RGGYQLLRWD | 2965 |
| | | | AQKFQG | | VGLLRSAFDI | |
| 149369 | GDSVSSNSA | 2886 | RTYYRSKWYS | 2926 | SSPEGLFLYWF | 2966 |
| | AWN | | FYAISLKS | | DP | |
| BCMA_EBB- | GFTFSSYAMS | 2887 | AISGSGGSTYY | 2927 | VEGSGSLDY | 2967 |
| C1978-A4 | | | ADSVKG | | | |
| BCMA_EBB- | GITFSRYPMS | 2888 | GISDSGVSTYY | 2928 | RAGSEASDI | 2968 |
| C1978-G1 | | | ADSAKG | | | |
| BCMA_EBB- | GFTFSSYAMS | 2889 | AISGSGGSTYY | 2929 | ATYKRELRYY | 2969 |
| C1979-C1 | | | ADSVKG | | YGMDV | |
| BCMA_EBB- | GFTFSSYAMS | 2890 | AISGSGGSTYY | 2930 | ATYKRELRYY | 2970 |
| C1978-C7 | | | ADSVKG | | YGMDV | |
| BCMA_EBB- | GFTFDDYA | 2891 | GISWNSGSIGY | 2931 | VGKAVPDV | 2971 |
| C1978- | MH | | ADSVKG | | | |
| D10 | | | | | | |
| BCMA_EBB- | GFTFDDYA | 2892 | SINWKGNSLA | 2932 | HQGVAYYNYA | 2972 |
| C1979- | MH | | YGDSVKG | | MDV | |
| C12 | | | | | | |
| BCMA_EBB- | GFTFSSYAMS | 2893 | AISGSGGSTYY | 2933 | VVRDGMDV | 2973 |
| C1980-G4 | | | ADSVKG | | | |
| CMA_EBB- | GFTFSSYAMS | 2894 | AISGSGGSTYY | 2934 | IPQTGTFDY | 2974 |
| C1980-D2 | | | ADSVKG | | | |
| BCMA_EBB- | GFTFSSYAMS | 2895 | AISGSGGSTYY | 2935 | ANYKRELRYY | 2975 |
| C1978- | | | ADSVKG | | YGMDV | |
| A10 | | | | | | |
| BCMA_EBB- | GFSFSSYAMS | 2896 | AISGSGGSTYY | 2936 | ALVGATGAFDI | 2976 |
| C1978-D4 | | | ADSVKG | | | |
| BCMA_EBB- | GFTFSSYAMS | 2897 | AISGSGGSTYY | 2937 | WFGEGFDP | 2977 |
| C1980-A2 | | | ADSVKG | | | |
| BCMA_EBB- | GFTFSSYAMS | 2898 | AISGSGGSTYY | 2938 | VGYDSSGYYR | 2978 |
| C1981-C3 | | | ADSVKG | | DYYGMDV | |
| BCMA_EBB- | GFTFSSYAMS | 2899 | AISGSGGSTYY | 2939 | MGWSSGYLGA | 2979 |
| C1978-G4 | | | ADSVKG | | FDI | |
| A7D12.2 | GYTFTNFG | 2900 | WINTYTGESY | 2940 | GEIYYGYDGGF | 2980 |
| | MN | | FADDFKG | | AY | |
| C11D5.3 | GYTFTDYSIN | 2901 | WINTETREPA | 2941 | DYSYAMDY | 2981 |
| | | | YAYDFRG | | | |
| C12A3.2 | GYTFRHYS | 2902 | RINTESGVPIY | 2942 | DYLYSLDF | 2982 |
| | MN | | ADDFKG | | | |
| C13F12.1 | GYTFTHYS | 2903 | RINTETGEPLY | 2943 | DYLYSCDY | 2983 |
| | MN | | ADDFKG | | | |

TABLE 4J
Light Chain Variable Domain CDRs according to a
combination of the Kabat numbering scheme (Kabat
et al. (1991), “Sequences of Proteins of Immunological
Interest,” 5th Ed. Public Health Service,
National Institutes of Health, Bethesda, MD)
and the Chothia numbering scheme (Al-Lazikani
et al., (1997) JMB 273, 927-948).

| Candidate | LCDR1 | ID | LCDR2 | ID | LCDR3 | ID |
|---|---|---|---|---|---|---|
| 139109 | RASQSISSYLN | 3994 | AASSLQS | 1034 | QQSYSTPYT | 3074 |
| 139103 | RASQSISSSFLA | 3984 | GASRRAT | 3024 | QQYHSSPS | 3064 |
| | | | | | WT | |
| 139105 | RSSQSLLHSNGYNY | 3985 | LGSNRAS | 3025 | MQALQTPYT | 3065 |
| | LD | | | | | |
| 139111 | KSSQSLLRNDGKTPLY | 3986 | EVSNRFS | 3026 | MQNIQFPS | 3066 |
| 139100 | RSSQSLLHSNGYNY | 3987 | LGSKRAS | 3027 | MQALQTPYT | 3067 |
| | LN | | | | | |
| 139101 | RASQSISSYLN | 3988 | GASTLAS | 3028 | QQSYKRAS | 3068 |
| 139102 | RSSQSLLYSNGYNY | 3989 | LGSNRAS | 3029 | MQGRQFPYS | 3069 |
| | VD | | | | | |
| 139104 | RASQSVSSNLA | 3990 | GASTRAS | 3030 | QQYGSSLT | 3070 |
| 139106 | RASQSVSSKLA | 3991 | GASIRAT | 3031 | QQYGSSSWT | 3071 |
| 139107 | RASQSVGSTNLA | 3992 | DASNRAT | 3032 | QQYGSSPP | 3072 |
| | | | | | WT | |
| 139108 | RASQSISSYLN | 3993 | AASSLQS | 3033 | QQSYTLA | 3073 |
| 139110 | KSSESLVHNSGKTY | 3995 | EVSNRDS | 3035 | MQGTHWP | 3075 |
| | LN | | | | GT | |
| 139112 | QASEDINKFLN | 3996 | DASTLQT | 3036 | QQYESLPLT | 3076 |
| 139113 | RASQSVGSNLA | 3997 | GASTRAT | 3037 | QQYNDWLP | 3077 |
| | | | | | VT | |
| 139114 | RASQSIGSSSLA | 3998 | GASSRAS | 3038 | QQYAGSPPFT | 3078 |
| 149362 | KASQDIDDAMN | 3999 | SATSPVP | 3039 | LQHDNFPLT | 3079 |
| 149363 | RASQDIYNNLA | 3000 | AANKSQS | 3040 | QHYYRFPYS | 3080 |
| 149364 | RSSQSLLHSNGYNY | 3001 | LGSNRAS | 3041 | MQALQTPYT | 3081 |
| | LD | | | | | |
| 149365 | GGNNIGTKSVH | 3002 | DDSVRPS | 3042 | QVWDSDSE | 3082 |
| | | | | | HVV | |
| 149366 | SGDGLSKKYVS | 3003 | RDKERPS | 3043 | QAWDDTTVV | 3083 |
| 149367 | RASQGIRNWLA | 3004 | AASNLQS | 3044 | QKYNSAPFT | 3084 |
| 149368 | GGNNIGSKSVH | 3005 | GKNNRPS | 3045 | SSRDSSGDH | 3085 |
| | | | | | LRV | |
| 149369 | QGDSLGNYYAT | 3006 | GTNNRPS | 3046 | NSRDSSGH | 3086 |
| | | | | | HLL | |
| BCMA_EBB- | RASQSVSSAYLA | 3007 | GASTRAT | 3047 | QHYGSSFN | 3087 |
| C1978- | | | | | GSSLFT | |
| A4 | | | | | | |
| BCMA_EBB- | RASQSVSNSLA | 3008 | DASSRAT | 3048 | QQFGTSSGLT | 3088 |
| C1978- | | | | | | |
| G1 | | | | | | |
| BCMA_EBB- | RASQSVSSSFLA | 3009 | GASSRAT | 3049 | QQYHSSPS | 3089 |
| C1979- | | | | | WT | |
| C1 | | | | | | |
| BCMA_EBB- | RASQSVSTTFLA | 3010 | GSSNRAT | 3050 | QQYHSSPS | 3090 |
| C1978- | | | | | WT | |
| C7 | | | | | | |
| BCMA_EBB- | RASQSISSYLN | 3011 | AASSLQS | 3051 | QQSYSTPYS | 3091 |
| C1978- | | | | | | |
| D10 | | | | | | |
| BCMA_EBB- | RATQSIGSSFLA | 3012 | GASQRAT | 3052 | QHYESSPS | 3092 |
| C1979- | | | | | WT | |
| C12 | | | | | | |
| BCMA_EBB- | RASQSVSSSYLA | 3013 | GASSRAT | 3053 | QQYGSPPRFT | 3093 |
| C1980- | | | | | | |
| G4 | | | | | | |
| BCMA_EBB- | RASQSVSSSYLA | 3014 | GASSRAT | 3054 | QHYGSSPS | 3094 |
| C1980- | | | | | WT | |
| D2 | | | | | | |
| BCMA_EBB- | RASQRVASNYLA | 3015 | GASSRAT | 3055 | QHYDSSPS | 3095 |
| C1978- | | | | | WT | |
| A10 | | | | | | |
| BCMA_EBB- | RASQSLSSNFLA | 3016 | GASNWAT | 3056 | QYYGTSPM | 3096 |
| C1978- | | | | | YT | |
| D4 | | | | | | |
| BCMA_EBB- | RSSQSLLHSNGYNY | 3017 | LGSNRAS | 3057 | MQALQTPLT | 3097 |
| C1980- | LD | | | | | |
| A2 | | | | | | |
| BCMA_EBB- | RASQSVSSSYLA | 3018 | GTSSRAT | 3058 | QHYGNSPP | 3098 |
| C1981- | | | | | KFT | |
| C3 | | | | | | |
| BCMA_EBB- | RASQSVASSFLA | 3019 | GASGRAT | 3059 | QHYGGSPR | 3099 |
| C1978- | | | | | LT | |
| G4 | | | | | | |
| A7D12.2 | RASQDVNTAVS | 3020 | SASYRYT | 3060 | QQHYSTPWT | 3100 |
| C11D5.3 | RASESVSVIGAHLIH | 3021 | LASNLET | 3061 | LQSRIFPRT | 3101 |
| C12A3.2 | RASESVTILGSHLIY | 3022 | LASNVQT | 3062 | LQSRTIPRT | 3102 |
| C13F12.1 | RASESVTILGSHLIY | 3023 | LASNVQT | 3063 | LQSRTIPRT | 3103 |

### Chimeric Antigen Receptor (CAR)

The present invention encompasses a recombinant DNA construct comprising sequences encoding a CAR, wherein the CAR comprises an antigen binding domain (e.g., antibody or antibody fragment, TCR or TCR fragment) that binds specifically to a cancer associated antigen described herein, wherein the sequence of the antigen binding domain is contiguous with and in the same reading frame as a nucleic acid sequence encoding an intracellular signaling domain. The intracellular signaling domain can comprise a costimulatory signaling domain and/or a primary signaling domain, e.g., a zeta chain. The costimulatory signaling domain refers to a portion of the CAR comprising at least a portion of the intracellular domain of a costimulatory molecule.

In specific aspects, a CAR construct of the invention comprises a scFv domain, wherein the scFv may be preceded by an optional leader sequence such as provided in SEQ ID NO: 2, and followed by an optional hinge sequence such as provided in SEQ ID NO:4 or SEQ ID NO:6 or SEQ ID NO:8 or SEQ ID NO:10, a transmembrane region such as provided in SEQ ID NO:12, an intracellular signalling domain that includes SEQ ID NO:14 or SEQ ID NO:16 and a CD3 zeta sequence that includes SEQ ID NO:18 or SEQ ID NO:20, e.g., wherein the domains are contiguous with and in the same reading frame to form a single fusion protein.

In one aspect, an exemplary CAR constructs comprise an optional leader sequence (e.g., a leader sequence described herein), an extracellular antigen binding domain (e.g., an antigen binding domain described herein), a hinge (e.g., a hinge region described herein), a transmembrane domain (e.g., a transmembrane domain described herein), and an intracellular stimulatory domain (e.g., an intracellular stimulatory domain described herein). In one aspect, an exemplary CAR construct comprises an optional leader sequence (e.g., a leader sequence described herein), an extracellular antigen binding domain (e.g., an antigen binding domain described herein), a hinge (e.g., a hinge region described herein), a transmembrane domain (e.g., a transmembrane domain described herein), an intracellular costimulatory signaling domain (e.g., a costimulatory signaling domain described herein) and/or an intracellular primary signaling domain (e.g., a primary signaling domain described herein).

An exemplary leader sequence is provided as SEQ ID NO: 2. An exemplary hinge/spacer sequence is provided as SEQ ID NO: 4 or SEQ ID NO:6 or SEQ ID NO:8 or SEQ ID NO:10. An exemplary transmembrane domain sequence is provided as SEQ ID NO:12. An exemplary sequence of the intracellular signaling domain of the 4-1BB protein is provided as SEQ ID NO: 14. An exemplary sequence of the intracellular signaling domain of CD27 is provided as SEQ ID NO:16. An exemplary CD3zeta domain sequence is provided as SEQ ID NO: 18 or SEQ ID NO:20.

In one aspect, the present invention encompasses a recombinant nucleic acid construct comprising a nucleic acid molecule encoding a CAR, wherein the nucleic acid molecule comprises the nucleic acid sequence encoding an antigen binding domain, e.g., described herein, that is contiguous with and in the same reading frame as a nucleic acid sequence encoding an intracellular signaling domain.

In one aspect, the present invention encompasses a recombinant nucleic acid construct comprising a nucleic acid molecule encoding a CAR, wherein the nucleic acid molecule comprises a nucleic acid sequence encoding an antigen binding domain, wherein the sequence is contiguous with and in the same reading frame as the nucleic acid sequence encoding an intracellular signaling domain. An exemplary intracellular signaling domain that can be used in the CAR includes, but is not limited to, one or more intracellular signaling domains of, e.g., CD3-zeta, CD28, CD27, 4-1BB, and the like. In some instances, the CAR can comprise any combination of CD3-zeta, CD28, 4-1BB, and the like.

The nucleic acid sequences coding for the desired molecules can be obtained using recombinant methods known in the art, such as, for example by screening libraries from cells expressing the nucleic acid molecule, by deriving the nucleic acid molecule from a vector known to include the same, or by isolating directly from cells and tissues containing the same, using standard techniques. Alternatively, the nucleic acid of interest can be produced synthetically, rather than cloned.

The present invention includes retroviral and lentiviral vector constructs expressing a CAR that can be directly transduced into a cell.

The present invention also includes an RNA construct that can be directly transfected into a cell. A method for generating mRNA for use in transfection involves in vitro transcription (IVT) of a template with specially designed primers, followed by polyA addition, to produce a construct containing 3′ and 5′ untranslated sequence (“UTR”) (e.g., a 3′ and/or 5′ UTR described herein), a 5′ cap (e.g., a 5′ cap described herein) and/or Internal Ribosome Entry Site (IRES) (e.g., an IRES described herein), the nucleic acid to be expressed, and a polyA tail, typically 50-2000 bases in length (SEQ ID NO:32). RNA so produced can efficiently transfect different kinds of cells. In one embodiment, the template includes sequences for the CAR. In an embodiment, an RNA CAR vector is transduced into a cell, e.g., a T cell or a NK cell, by electroporation.

Antigen Binding Domain

In one aspect, the CAR of the invention comprises a target-specific binding element otherwise referred to as an antigen binding domain. The choice of moiety depends upon the type and number of ligands that define the surface of a target cell. For example, the antigen binding domain may be chosen to recognize a ligand that acts as a cell surface marker on target cells associated with a particular disease state. Thus, examples of cell surface markers that may act as ligands for the antigen binding domain in a CAR of the invention include those associated with viral, bacterial and parasitic infections, autoimmune disease and cancer cells.

In one aspect, the CAR-mediated T-cell response can be directed to an antigen of interest by way of engineering an antigen binding domain that specifically binds a desired antigen into the CAR.

In one aspect, the portion of the CAR comprising the antigen binding domain comprises an antigen binding domain that targets a tumor antigen, e.g., a tumor antigen described herein.

The antigen binding domain can be any domain that binds to the antigen including but not limited to a monoclonal antibody, a polyclonal antibody, a recombinant antibody, a human antibody, a humanized antibody, and a functional fragment thereof, including but not limited to a single-domain antibody such as a heavy chain variable domain (VH), a light chain variable domain (VL) and a variable domain (VHH) of camelid derived nanobody, and to an alternative scaffold known in the art to function as antigen binding domain, such as a recombinant fibronectin domain, a T cell receptor (TCR), or a fragment there of, e.g., single chain TCR, and the like. In some instances, it is beneficial for the antigen binding domain to be derived from the same species in which the CAR will ultimately be used in. For example, for use in humans, it may be beneficial for the antigen binding domain of the CAR to comprise human or humanized residues for the antigen binding domain of an antibody or antibody fragment.

In one embodiment, the CD19 CAR is a CD19 CAR described in U.S. Pat. Nos. 8,399,645; 7,446,190; Xu et al., Leuk Lymphoma. 2013 54(2):255-260(2012); Cruz et al., Blood 122(17):2965-2973 (2013); Brentjens et al., Blood, 118(18):4817-4828 (2011); Kochenderfer et al., Blood 116(20):4099-102 (2010); Kochenderfer et al., Blood 122 (25):4129-39(2013); or 16th Annu Meet Am Soc Gen Cell Ther (ASGCT) (May 15-18, Salt Lake City) 2013, Abst 10 (each of which is herein incorporated by reference in their entirety). In one embodiment, an antigen binding domain against CD19 is an antigen binding portion, e.g., CDRs, of a CAR, antibody or antigen-binding fragment thereof described in, e.g., PCT publication WO2012/079000 (incorporated herein by reference in its entirety). In one embodiment, an antigen binding domain against CD19 is an antigen binding portion, e.g., CDRs, of a CAR, antibody or antigen-binding fragment thereof described in, e.g., PCT publication WO2014/153270; Kochenderfer, J. N. et al., J. Immunother. 32 (7), 689-702 (2009); Kochenderfer, J. N., et al., Blood, 116 (20), 4099-4102 (2010); PCT publication WO2014/031687; Bejcek, Cancer Research, 55, 2346-2351, 1995; or U.S. Pat. No. 7,446,190 (each of which is herein incorporated by reference in their entirety).

In one embodiment, the antigen binding domain against mesothelin is or may be derived from an antigen binding domain, e.g., CDRs, scFv, or VH and VL, of an antibody, antigen-binding fragment or CAR described in, e.g., PCT publication WO2015/090230 (In one embodiment the CAR is a CAR described in WO2015/090230, the contents of which are incorporated herein in their entirety). In some embodiments, the antigen binding domain against mesothelin is or is derived from an antigen binding portion, e.g., CDRs, scFv, or VH and VL, of an antibody, antigen-binding fragment, or CAR described in, e.g., PCT publication WO1997/025068, WO1999/028471, WO2005/014652, WO2006/099141, WO2009/045957, WO2009/068204, WO2013/142034, WO2013/040557, or WO2013/063419 (each of which is herein incorporated by reference in their entirety).

In one embodiment, an antigen binding domain against CD123 is or is derived from an antigen binding portion, e.g., CDRs, scFv or VH and VL, of an antibody, antigen-binding fragment or CAR described in, e.g., PCT publication WO2014/130635 (incorporated herein by referenc in its entirety). In one embodiment, an antigen binding domain against CD123 is or is derived from an antigen binding portion, e.g., CDRs, scFv or VH and VL, of an antibody, antigen-binding fragment or CAR described in, e.g., PCT publication WO2016/028896 (incorporated herein by referenc in its entirety); in some embodiments, the CAR is a CAR described in WO2016/028896. In one embodiment, an antigen binding domain against CD123 is or is derived from an antigen binding portion, e.g., CDRs, scFv, or VL and VH, of an antibody, antigen-binding fragment, or CAR described in, e.g., PCT publication WO1997/024373, WO2008/127735 (e.g., a CD123 binding domain of 26292, 32701, 37716 or 32703), WO2014/138805 (e.g., a CD123 binding domain of CSL362), WO2014/138819, WO2013/173820, WO2014/144622, WO2001/66139, WO2010/126066 (e.g., the CD123 binding domain of any of Old4, Old5, Old17, Old19, New102, or Old6), WO2014/144622, or US2009/0252742 (each of which is incorporated herein by reference in its entirety).

In one embodiment, an antigen binding domain against CD22 is an antigen binding portion, e.g., CDRs, of an antibody described in, e.g., Haso et al., Blood, 121(7): 1165-1174 (2013); Wayne et al., Clin Cancer Res 16(6): 1894-1903 (2010); Kato et al., Leuk Res 37(1):83-88 (2013); Creative BioMart (creativebiomart.net): MOM-18047-S(P).

In one embodiment, an antigen binding domain against CS-1 is an antigen binding portion, e.g., CDRs, of Elotuzumab (BMS), see e.g., Tai et al., 2008, Blood 112(4):1329-37; Tai et al., 2007, Blood. 110(5):1656-63.

In one embodiment, an antigen binding domain against CLL-1 is an antigen binding portion, e.g., CDRs or VH and VL, of an antibody, antigen-binding fragment or CAR described in, e.g., PCT publication WO2016/014535, the contents of which are incorporated herein in their entirety. In one embodiment, an antigen binding domain against CLL-1 is an antigen binding portion, e.g., CDRs, of an antibody available from R&D, ebiosciences, Abcam, for example, PE-CLL1-hu Cat #353604 (BioLegend); and PE-CLL1 (CLEC12A) Cat #562566 (BD).

In one embodiment, an antigen binding domain against CD33 is an antigen binding portion, e.g., CDRs, of an antibody described in, e.g., Bross et al., Clin Cancer Res 7(6):1490-1496 (2001) (Gemtuzumab Ozogamicin, hP67.6), Caron et al., Cancer Res 52(24):6761-6767 (1992) (Lintuzumab, HuM195), Lapusan et al., Invest New Drugs 30(3):1121-1131 (2012) (AVE9633), Aigner et al., Leukemia 27(5): 1107-1115 (2013) (AMG330, CD33 BiTE), Dutour et al., Adv hematol 2012:683065 (2012), and Pizzitola et al., Leukemia doi:10.1038/Lue.2014.62 (2014). Exemplary CAR molecules that target CD33 are described herein, and are provided in WO2016/014576, e.g., in Table 2 of WO2016/014576 (incorporated by reference in its entirety).

In one embodiment, an antigen binding domain against GD2 is an antigen binding portion, e.g., CDRs, of an antibody described in, e.g., Mujoo et al., Cancer Res. 47(4):1098-1104 (1987); Cheung et al., Cancer Res 45(6):2642-2649 (1985), Cheung et al., J Clin Oncol 5(9):1430-1440 (1987), Cheung et al., J Clin Oncol 16(9):3053-3060 (1998), Handgretinger et al., Cancer Immunol Immunother 35(3):199-204 (1992). In some embodiments, an antigen binding domain against GD2 is an antigen binding portion of an antibody selected from mAb 14.18, 14G2a, ch14.18, hu14.18, 3F8, hu3F8, 3G6, 8B6, 60C3, 10B8, ME36.1, and 8H9, see e.g., WO2012033885, WO2013040371, WO2013192294, WO2013061273, WO2013123061, WO2013074916, and WO201385552. In some embodiments, an antigen binding domain against GD2 is an antigen binding portion of an antibody described in US Publication No.: 20100150910 or PCT Publication No.: WO 2011160119.

In one embodiment, an antigen binding domain against BCMA is an antigen binding portion, e.g., CDRs, of an antibody described in, e.g., WO2012163805, WO200112812, and WO2003062401. In some embodiments, additional exemplary BCMA CAR constructs are generated using an antigen binding domain, e.g., CDRs, scFv, or VH and VL sequences from PCT Publication WO2012/0163805 (the contents of which are hereby incorporated by reference in its entirety). In some embodiments, additional exemplary BCMA CAR constructs are generated using an antigen binding domain, e.g., CDRs, scFv, or VH and VL sequences from PCT Publication WO2016/014565 (the contents of which are hereby incorporated by reference in its entirety). In some embodiments, additional exemplary BCMA CAR constructs are generated using an antigen binding domain, e.g., CDRs, scFv, or VH and VL sequences from PCT Publication WO2014/122144 (the contents of which are hereby incorporated by reference in its entirety). In some embodiments, additional exemplary BCMA CAR constructs are generated using the CAR molecules, and/or the BCMA binding domains (e.g., CDRs, scFv, or VH and VL sequences) from PCT Publication WO2016/014789 (the contents of which are hereby incorporated by reference in its entirety). In some embodiments, additional exemplary BCMA CAR constructs are generated using the CAR molecules, and/or the BCMA binding domains (e.g., CDRs, scFv, or VH and VL sequences) from PCT Publication WO2014/089335 (the contents of which are hereby incorporated by reference in its entirety). In some embodiments, additional exemplary BCMA CAR constructs are generated using the CAR molecules, and/or the BCMA binding domains (e.g., CDRs, scFv, or VH and VL sequences) from PCT Publication WO2014/140248 (the contents of which are hereby incorporated by reference in its entirety).

In one embodiment, an antigen binding domain against Tn antigen is an antigen binding portion, e.g., CDRs, of an antibody described in, e.g., US 2014/0178365, U.S. Pat. No. 8,440,798, Brooks et al., PNAS 107(22):10056-10061 (2010), and Stone et al., Oncolmmunology 1(6):863-873(2012).

In one embodiment, an antigen binding domain against PSMA is an antigen binding portion, e.g., CDRs, of an antibody described in, e.g., Parker et al., Protein Expr Purif 89(2):136-145 (2013), US 20110268656 (J591 ScFv); Frigerio et al, European J Cancer 49(9):2223-2232 (2013) (scFvD2B); WO 2006125481 (mAbs 3/A12, 3/E7 and 3/F11) and single chain antibody fragments (scFv A5 and D7).

In one embodiment, an antigen binding domain against ROR1 is an antigen binding portion, e.g., CDRs, of an antibody described in, e.g., Hudecek et al., Clin Cancer Res 19(12):3153-3164 (2013); WO 2011159847; and US20130101607.

In one embodiment, an antigen binding domain against FLT3 is an antigen binding portion, e.g., CDRs, of an antibody described in, e.g., WO2011076922, U.S. Pat. No. 5,777,084, EP0754230, US20090297529, and several commercial catalog antibodies (R&D, ebiosciences, Abcam).

In one embodiment, an antigen binding domain against TAG72 is an antigen binding portion, e.g., CDRs, of an antibody described in, e.g., Hombach et al., Gastroenterology 113(4):1163-1170 (1997); and Abcam ab691.

In one embodiment, an antigen binding domain against FAP is an antigen binding portion, e.g., CDRs, of an antibody described in, e.g., Ostermann et al., Clinical Cancer Research 14:4584-4592 (2008) (FAPS), US Pat. Publication No. 2009/0304718; sibrotuzumab (see e.g., Hofheinz et al., Oncology Research and Treatment 26(1), 2003); and Tran et al., J Exp Med 210(6):1125-1135 (2013).

In one embodiment, an antigen binding domain against CD38 is an antigen binding portion, e.g., CDRs, of daratumumab (see, e.g., Groen et al., Blood 116(21):1261-1262 (2010); MOR202 (see, e.g., U.S. Pat. No. 8,263,746); or antibodies described in U.S. Pat. No. 8,362,211.

In one embodiment, an antigen binding domain against CD44v6 is an antigen binding portion, e.g., CDRs, of an antibody described in, e.g., Casucci et al., Blood 122(20):3461-3472 (2013).

In one embodiment, an antigen binding domain against CEA is an antigen binding portion, e.g., CDRs, of an antibody described in, e.g., Chmielewski et al., Gastoenterology 143(4):1095-1107 (2012).

In one embodiment, an antigen binding domain against EPCAM is an antigen binding portion, e.g., CDRS, of an antibody selected from MT110, EpCAM-CD3 bispecific Ab (see, e.g., clinicaltrials.gov/ct2/show/NCT00635596); Edrecolomab; 3622W94; ING-1; and adecatumumab (MT201).

In one embodiment, an antigen binding domain against PRSS21 is an antigen binding portion, e.g., CDRs, of an antibody described in U.S. Pat. No. 8,080,650.

In one embodiment, an antigen binding domain against B7H3 is an antigen binding portion, e.g., CDRs, of an antibody MGA271 (Macrogenics).

In one embodiment, an antigen binding domain against KIT is an antigen binding portion, e.g., CDRs, of an antibody described in, e.g., U.S. Pat. No. 7,915,391, US20120288506, and several commercial catalog antibodies.

In one embodiment, an antigen binding domain against IL-13Ra2 is an antigen binding portion, e.g., CDRs, of an antibody described in, e.g., WO2008/146911, WO2004087758, several commercial catalog antibodies, and WO2004087758.

In one embodiment, an antigen binding domain against CD30 is an antigen binding portion, e.g., CDRs, of an antibody described in, e.g., U.S. Pat. No. 7,090,843 B1, and EP0805871.

In one embodiment, an antigen binding domain against GD3 is an antigen binding portion, e.g., CDRs, of an antibody described in, e.g., U.S. Pat. Nos. 7,253,263; 8,207,308; US 20120276046; EP1013761; WO2005035577; and U.S. Pat. No. 6,437,098.

In one embodiment, an antigen binding domain against CD171 is an antigen binding portion, e.g., CDRs, of an antibody described in, e.g., Hong et al., J Immunother 37(2):93-104 (2014).

In one embodiment, an antigen binding domain against IL-11Ra is an antigen binding portion, e.g., CDRs, of an antibody available from Abcam (cat # ab55262) or Novus Biologicals (cat # EPR5446). In another embodiment, an antigen binding domain again IL-11Ra is a peptide, see, e.g., Huang et al., Cancer Res 72(1):271-281 (2012).

In one embodiment, an antigen binding domain against PSCA is an antigen binding portion, e.g., CDRs, of an antibody described in, e.g., Morgenroth et al., Prostate 67(10):1121-1131 (2007) (scFv 7F5); Nejatollahi et al., J of Oncology 2013(2013), article ID 839831 (scFv C5-II); and US Pat Publication No. 20090311181.

In one embodiment, an antigen binding domain against VEGFR2 is an antigen binding portion, e.g., CDRs, of an antibody described in, e.g., Chinnasamy et al., J Clin Invest 120(11):3953-3968 (2010).

In one embodiment, an antigen binding domain against LewisY is an antigen binding portion, e.g., CDRs, of an antibody described in, e.g., Kelly et al., Cancer Biother Radiopharm 23(4):411-423 (2008) (hu3S193 Ab (scFvs)); Dolezal et al., Protein Engineering 16(1):47-56 (2003) (NC10 scFv).

In one embodiment, an antigen binding domain against CD24 is an antigen binding portion, e.g., CDRs, of an antibody described in, e.g., Maliar et al., Gastroenterology 143(5):1375-1384 (2012).

In one embodiment, an antigen binding domain against PDGFR-beta is an antigen binding portion, e.g., CDRs, of an antibody Abcam ab32570.

In one embodiment, an antigen binding domain against SSEA-4 is an antigen binding portion, e.g., CDRs, of antibody MC813 (Cell Signaling), or other commercially available antibodies.

In one embodiment, an antigen binding domain against CD20 is an antigen binding portion, e.g., CDRs, of the antibody Rituximab, Ofatumumab, Ocrelizumab, Veltuzumab, or GA101.

In one embodiment, an antigen binding domain against Folate receptor alpha is an antigen binding portion, e.g., CDRs, of the antibody IMGN853, or an antibody described in US20120009181; U.S. Pat. No. 4,851,332, LK26: U.S. Pat. No. 5,952,484.

In one embodiment, an antigen binding domain against ERBB2 (Her2/neu) is an antigen binding portion, e.g., CDRs, of the antibody trastuzumab, or pertuzumab.

In one embodiment, an antigen binding domain against MUC1 is an antigen binding portion, e.g., CDRs, of the antibody SAR566658.

In one embodiment, the antigen binding domain against EGFR is antigen binding portion, e.g., CDRs, of the antibody cetuximab, panitumumab, zalutumumab, nimotuzumab, or matuzumab. In one embodiment, the antigen binding domain against EGFRvIII is or may be derived from an antigen binding domain, e.g., CDRs, scFv, or VH and VL, of an antibody, antigen-binding fragment or CAR described in, e.g., PCT publication WO2014/130657 (In one embodiment the CAR is a CAR described in WO2014/130657, the contents of which are incorporated herein in their entirety).

In one embodiment, an antigen binding domain against NCAM is an antigen binding portion, e.g., CDRs, of the antibody clone 2-2B: MAB5324 (EMD Millipore)

In one embodiment, an antigen binding domain against Ephrin B2 is an antigen binding portion, e.g., CDRs, of an antibody described in, e.g., Abengozar et al., Blood 119(19):4565-4576 (2012).

In one embodiment, an antigen binding domain against IGF-I receptor is an antigen binding portion, e.g., CDRs, of an antibody described in, e.g., U.S. Pat. No. 8,344,112 B2; EP2322550 A1; WO 2006/138315, or PCT/US2006/022995.

In one embodiment, an antigen binding domain against CAIX is an antigen binding portion, e.g., CDRs, of the antibody clone 303123 (R&D Systems).

In one embodiment, an antigen binding domain against LMP2 is an antigen binding portion, e.g., CDRs, of an antibody described in, e.g., U.S. Pat. No. 7,410,640, or US20050129701.

In one embodiment, an antigen binding domain against gp100 is an antigen binding portion, e.g., CDRs, of the antibody HMB45, NKIbetaB, or an antibody described in WO2013165940, or US20130295007.

In one embodiment, an antigen binding domain against tyrosinase is an antigen binding portion, e.g., CDRs, of an antibody described in, e.g., U.S. Pat. No. 5,843,674; or US19950504048.

In one embodiment, an antigen binding domain against EphA2 is an antigen binding portion, e.g., CDRs, of an antibody described in, e.g., Yu et al., Mol Ther 22(1):102-111 (2014).

In one embodiment, an antigen binding domain against GD3 is an antigen binding portion, e.g., CDRs, of an antibody described in, e.g., U.S. Pat. Nos. 7,253,263; 8,207,308; US 20120276046; EP1013761 A3; 20120276046; WO2005035577; or U.S. Pat. No. 6,437,098.

In one embodiment, an antigen binding domain against fucosyl GM1 is an antigen binding portion, e.g., CDRs, of an antibody described in, e.g., US20100297138; or WO2007/067992.

In one embodiment, an antigen binding domain against sLe is an antigen binding portion, e.g., CDRs, of the antibody G193 (for lewis Y), see Scott A M et al, Cancer Res 60: 3254-61 (2000), also as described in Neeson et al, J Immunol May 2013 190 (Meeting Abstract Supplement) 177.10.

In one embodiment, an antigen binding domain against GM3 is an antigen binding portion, e.g., CDRs, of the antibody CA 2523449 (mAb 14F7).

In one embodiment, an antigen binding domain against HMWMAA is an antigen binding portion, e.g., CDRs, of an antibody described in, e.g., Kmiecik et al., Oncoimmunology 3(1):e27185 (2014) (PMID: 24575382) (mAb9.2.27); U.S. Pat. No. 6,528,481; WO2010033866; or US 20140004124.

In one embodiment, an antigen binding domain against o-acetyl-GD2 is an antigen binding portion, e.g., CDRs, of the antibody 8B6.

In one embodiment, an antigen binding domain against TEM1/CD248 is an antigen binding portion, e.g., CDRs, of an antibody described in, e.g., Marty et al., Cancer Lett 235(2):298-308 (2006); Zhao et al., J Immunol Methods 363(2):221-232 (2011).

In one embodiment, an antigen binding domain against CLDN6 is an antigen binding portion, e.g., CDRs, of the antibody IMAB027 (Ganymed Pharmaceuticals), see e.g., clinicaltrial.gov/show/NCT02054351.

In one embodiment, an antigen binding domain against TSHR is an antigen binding portion, e.g., CDRs, of an antibody described in, e.g., U.S. Pat. Nos. 8,603,466; 8,501,415; or U.S. Pat. No. 8,309,693.

In one embodiment, an antigen binding domain against GPRC5D is an antigen binding portion, e.g., CDRs, of the antibody FAB6300A (R&D Systems); or LS-A4180 (Lifespan Biosciences).

In one embodiment, an antigen binding domain against CD97 is an antigen binding portion, e.g., CDRs, of an antibody described in, e.g., U.S. Pat. No. 6,846,911; de Groot et al., J Immunol 183(6):4127-4134 (2009); or an antibody from R&D:MAB3734.

In one embodiment, an antigen binding domain against ALK is an antigen binding portion, e.g., CDRs, of an antibody described in, e.g., Mino-Kenudson et al., Clin Cancer Res 16(5):1561-1571 (2010).

In one embodiment, an antigen binding domain against polysialic acid is an antigen binding portion, e.g., CDRs, of an antibody described in, e.g., Nagae et al., J Biol Chem 288(47):33784-33796 (2013).

In one embodiment, an antigen binding domain against PLAC1 is an antigen binding portion, e.g., CDRs, of an antibody described in, e.g., Ghods et al., Biotechnol Appl Biochem 2013 doi:10.1002/bab.1177.

In one embodiment, an antigen binding domain against GloboH is an antigen binding portion of the antibody VK9; or an antibody described in, e.g., Kudryashov V et al, Glycoconj J.15(3):243-9 (1998), Lou et al., Proc Natl Acad Sci USA 111(7):2482-2487 (2014); MBr1: Bremer E-G et al. J Biol Chem 259:14773-14777 (1984).

In one embodiment, an antigen binding domain against NY-BR-1 is an antigen binding portion, e.g., CDRs of an antibody described in, e.g., Jager et al., Appl Immunohistochem Mol Morphol 15(1):77-83 (2007).

In one embodiment, an antigen binding domain against WT-1 is an antigen binding portion, e.g., CDRs, of an antibody described in, e.g., Dao et al., Sci Transl Med 5(176):176ra33 (2013); or WO2012/135854.

In one embodiment, an antigen binding domain against MAGE-A1 is an antigen binding portion, e.g., CDRs, of an antibody described in, e.g., Willemsen et al., J Immunol 174(12):7853-7858 (2005) (TCR-like scFv).

In one embodiment, an antigen binding domain against sperm protein 17 is an antigen binding portion, e.g., CDRs, of an antibody described in, e.g., Song et al., Target Oncol 2013 Aug. 14 (PMID: 23943313); Song et al., Med Oncol 29(4):2923-2931 (2012).

In one embodiment, an antigen binding domain against Tie 2 is an antigen binding portion, e.g., CDRs, of the antibody AB33 (Cell Signaling Technology).

In one embodiment, an antigen binding domain against MAD-CT-2 is an antigen binding portion, e.g., CDRs, of an antibody described in, e.g., PMID: 2450952; U.S. Pat. No. 7,635,753.

In one embodiment, an antigen binding domain against Fos-related antigen 1 is an antigen binding portion, e.g., CDRs, of the antibody 12F9 (Novus Biologicals).

In one embodiment, an antigen binding domain against MelanA/MART1 is an antigen binding portion, e.g., CDRs, of an antibody described in, EP2514766 A2; or U.S. Pat. No. 7,749,719.

In one embodiment, an antigen binding domain against sarcoma translocation breakpoints is an antigen binding portion, e.g., CDRs, of an antibody described in, e.g., Luo et al, EMBO Mol. Med. 4(6):453-461 (2012).

In one embodiment, an antigen binding domain against TRP-2 is an antigen binding portion, e.g., CDRs, of an antibody described in, e.g., Wang et al, J Exp Med. 184(6):2207-16 (1996).

In one embodiment, an antigen binding domain against CYP1B1 is an antigen binding portion, e.g., CDRs, of an antibody described in, e.g., Maecker et al, Blood 102 (9): 3287-3294 (2003).

In one embodiment, an antigen binding domain against RAGE-1 is an antigen binding portion, e.g., CDRs, of the antibody MAB5328 (EMD Millipore).

In one embodiment, an antigen binding domain against human telomerase reverse transcriptase is an antigen binding portion, e.g., CDRs, of the antibody cat no: LS-B95-100 (Lifespan Biosciences)

In one embodiment, an antigen binding domain against intestinal carboxyl esterase is an antigen binding portion, e.g., CDRs, of the antibody 4F12: cat no: LS-B6190-50 (Lifespan Biosciences).

In one embodiment, an antigen binding domain against mut hsp70-2 is an antigen binding portion, e.g., CDRs, of the antibody Lifespan Biosciences: monoclonal: cat no: LS-C133261-100 (Lifespan Biosciences).

In one embodiment, an antigen binding domain against CD79a is an antigen binding portion, e.g., CDRs, of the antibody Anti-CD79a antibody [HM47/A9] (ab3121), available from Abcam; antibody CD79A Antibody #3351 available from Cell Signalling Technology; or antibody HPA017748-Anti-CD79A antibody produced in rabbit, available from Sigma Aldrich.

In one embodiment, an antigen binding domain against CD79b is an antigen binding portion, e.g., CDRs, of the antibody polatuzumab vedotin, anti-CD79b described in Dornan et al., “Therapeutic potential of an anti-CD79b antibody-drug conjugate, anti-CD79b-vc-MMAE, for the treatment of non-Hodgkin lymphoma” Blood. 2009 Sep. 24; 114(13):2721-9. doi: 10.1182/blood-2009-02-205500. Epub 2009 Jul. 24, or the bispecific antibody Anti-CD79b/CD3 described in “4507 Pre-Clinical Characterization of T Cell-Dependent Bispecific Antibody Anti-CD79b/CD3 As a Potential Therapy for B Cell Malignancies” Abstracts of 56^{th} ASH Annual Meeting and Exposition, San Francisco, Calif. Dec. 6-9 2014.

In one embodiment, an antigen binding domain against CD72 is an antigen binding portion, e.g., CDRs, of the antibody J3-109 described in Myers, and Uckun, “An anti-CD72 immunotoxin against therapy-refractory B-lineage acute lymphoblastic leukemia.” Leuk Lymphoma. 1995 June; 18(1-2):119-22, or anti-CD72 (10D6.8.1, mIgG1) described in Polson et al., “Antibody-Drug Conjugates for the Treatment of Non-Hodgkin's Lymphoma: Target and Linker-Drug Selection” Cancer Res Mar. 15, 2009 69; 2358.

In one embodiment, an antigen binding domain against LAIR1 is an antigen binding portion, e.g., CDRs, of the antibody ANT-301 LAIR1 antibody, available from ProSpec; or anti-human CD305 (LAIR1) Antibody, available from BioLegend.

In one embodiment, an antigen binding domain against FCAR is an antigen binding portion, e.g., CDRs, of the antibody CD89/FCARAntibody (Catalog #10414-H08H), available from Sino Biological Inc.

In one embodiment, an antigen binding domain against LILRA2 is an antigen binding portion, e.g., CDRs, of the antibody LILRA2 monoclonal antibody (M17), clone 3C7, available from Abnova, or Mouse Anti-LILRA2 antibody, Monoclonal (2D7), available from Lifespan Biosciences.

In one embodiment, an antigen binding domain against CD300LF is an antigen binding portion, e.g., CDRs, of the antibody Mouse Anti-CMRF35-like molecule 1 antibody, Monoclonal[UP-D2], available from BioLegend, or Rat Anti-CMRF35-like molecule 1 antibody, Monoclonal[234903], available from R&D Systems.

In one embodiment, an antigen binding domain against CLEC12A is an antigen binding portion, e.g., CDRs, of the antibody Bispecific T cell Engager (BiTE) scFv-antibody and ADC described in Noordhuis et al., “Targeting of CLEC12A In Acute Myeloid Leukemia by Antibody-Drug-Conjugates and Bispecific CLL-1×CD3 BiTE Antibody” 53^{rd} ASH Annual Meeting and Exposition, Dec. 10-13, 2011, and MCLA-117 (Merus).

In one embodiment, an antigen binding domain against BST2 (also called CD317) is an antigen binding portion, e.g., CDRs, of the antibody Mouse Anti-CD317 antibody, Monoclonal[3H4], available from Antibodies-Online or Mouse Anti-CD317 antibody, Monoclonal[696739], available from R&D Systems.

In one embodiment, an antigen binding domain against EMR2 (also called CD312) is an antigen binding portion, e.g., CDRs, of the antibody Mouse Anti-CD312 antibody, Monoclonal[LS-B8033] available from Lifespan Biosciences, or Mouse Anti-CD312 antibody, Monoclonal[494025] available from R&D Systems.

In one embodiment, an antigen binding domain against LY75 is an antigen binding portion, e.g., CDRs, of the antibody Mouse Anti-Lymphocyte antigen 75 antibody, Monoclonal[HD30] available from EMD Millipore or Mouse Anti-Lymphocyte antigen 75 antibody, Monoclonal[A15797] available from Life Technologies.

In one embodiment, an antigen binding domain against GPC3 is an antigen binding portion, e.g., CDRs, of the antibody hGC33 described in Nakano K, Ishiguro T, Konishi H, et al. Generation of a humanized anti-glypican 3 antibody by CDR grafting and stability optimization. Anticancer Drugs. 2010 November; 21(10):907-916, or MDX-1414, HN3, or YP7, all three of which are described in Feng et al., “Glypican-3 antibodies: a new therapeutic target for liver cancer.” FEBS Lett. 2014 Jan. 21; 588(2):377-82.

In one embodiment, an antigen binding domain against FCRL5 is an antigen binding portion, e.g., CDRs, of the anti-FcRL5 antibody described in Elkins et al., “FcRL5 as a target of antibody-drug conjugates for the treatment of multiple myeloma” Mol Cancer Ther. 2012 October; 11(10):2222-32.

In one embodiment, an antigen binding domain against IGLL1 is an antigen binding portion, e.g., CDRs, of the antibody Mouse Anti-Immunoglobulin lambda-like polypeptide 1 antibody, Monoclonal[AT1G4] available from Lifespan Biosciences, Mouse Anti-Immunoglobulin lambda-like polypeptide 1 antibody, Monoclonal[HSL11] available from BioLegend.

In one embodiment, the antigen binding domain comprises one, two three (e.g., all three) heavy chain CDRs, HC CDR1, HC CDR2 and HC CDR3, from an antibody listed above, and/or one, two, three (e.g., all three) light chain CDRs, LC CDR1, LC CDR2 and LC CDR3, from an antibody listed above. In one embodiment, the antigen binding domain comprises a heavy chain variable region and/or a variable light chain region of an antibody listed above.

In another aspect, the antigen binding domain comprises a humanized antibody or an antibody fragment. In some aspects, a non-human antibody is humanized, where specific sequences or regions of the antibody are modified to increase similarity to an antibody naturally produced in a human or fragment thereof. In one aspect, the antigen binding domain is humanized.

A humanized antibody can be produced using a variety of techniques known in the art, including but not limited to, CDR-grafting (see, e.g., European Patent No. EP 239,400; International Publication No. WO 91/09967; and U.S. Pat. Nos. 5,225,539, 5,530,101, and 5,585,089, each of which is incorporated herein in its entirety by reference), veneering or resurfacing (see, e.g., European Patent Nos. EP 592,106 and EP 519,596; Padlan, 1991, Molecular Immunology, 28(4/5):489-498; Studnicka et al., 1994, Protein Engineering, 7(6):805-814; and Roguska et al., 1994, PNAS, 91:969-973, each of which is incorporated herein by its entirety by reference), chain shuffling (see, e.g., U.S. Pat. No. 5,565,332, which is incorporated herein in its entirety by reference), and techniques disclosed in, e.g., U.S. Patent Application Publication No. US2005/0042664, U.S. Patent Application Publication No. US2005/0048617, U.S. Pat. Nos. 6,407,213, 5,766,886, International Publication No. WO 9317105, Tan et al., J. Immunol., 169:1119-25 (2002), Caldas et al., Protein Eng., 13(5):353-60 (2000), Morea et al., Methods, 20(3):267-79 (2000), Baca et al., J. Biol. Chem., 272(16):10678-84 (1997), Roguska et al., Protein Eng., 9(10):895-904 (1996), Couto et al., Cancer Res., 55 (23 Supp):5973s-5977s (1995), Couto et al., Cancer Res., 55(8):1717-22 (1995), Sandhu J S, Gene, 150(2):409-10 (1994), and Pedersen et al., J. Mol. Biol., 235(3):959-73 (1994), each of which is incorporated herein in its entirety by reference. Often, framework residues in the framework regions will be substituted with the corresponding residue from the CDR donor antibody to alter, for example improve, antigen binding. These framework substitutions are identified by methods well-known in the art, e.g., by modeling of the interactions of the CDR and framework residues to identify framework residues important for antigen binding and sequence comparison to identify unusual framework residues at particular positions. (See, e.g., Queen et al., U.S. Pat. No. 5,585,089; and Riechmann et al., 1988, Nature, 332:323, which are incorporated herein by reference in their entireties.)

A humanized antibody or antibody fragment has one or more amino acid residues remaining in it from a source which is nonhuman. These nonhuman amino acid residues are often referred to as “import” residues, which are typically taken from an “import” variable domain. As provided herein, humanized antibodies or antibody fragments comprise one or more CDRs from nonhuman immunoglobulin molecules and framework regions wherein the amino acid residues comprising the framework are derived completely or mostly from human germline. Multiple techniques for humanization of antibodies or antibody fragments are well-known in the art and can essentially be performed following the method of Winter and co-workers (Jones et al., Nature, 321:522-525 (1986); Riechmann et al., Nature, 332:323-327 (1988); Verhoeyen et al., Science, 239:1534-1536 (1988)), by substituting rodent CDRs or CDR sequences for the corresponding sequences of a human antibody, i.e., CDR-grafting (EP 239,400; PCT Publication No. WO 91/09967; and U.S. Pat. Nos. 4,816,567; 6,331,415; 5,225,539; 5,530,101; 5,585,089; 6,548,640, the contents of which are incorporated herein by reference herein in their entirety). In such humanized antibodies and antibody fragments, substantially less than an intact human variable domain has been substituted by the corresponding sequence from a nonhuman species. Humanized antibodies are often human antibodies in which some CDR residues and possibly some framework (FR) residues are substituted by residues from analogous sites in rodent antibodies. Humanization of antibodies and antibody fragments can also be achieved by veneering or resurfacing (EP 592,106; EP 519,596; Padlan, 1991, Molecular Immunology, 28(4/5):489-498; Studnicka et al., Protein Engineering, 7(6):805-814 (1994); and Roguska et al., PNAS, 91:969-973 (1994)) or chain shuffling (U.S. Pat. No. 5,565,332), the contents of which are incorporated herein by reference herein in their entirety.

The choice of human variable domains, both light and heavy, to be used in making the humanized antibodies is to reduce antigenicity. According to the so-called “best-fit” method, the sequence of the variable domain of a rodent antibody is screened against the entire library of known human variable-domain sequences. The human sequence which is closest to that of the rodent is then accepted as the human framework (FR) for the humanized antibody (Sims et al., J. Immunol., 151:2296 (1993); Chothia et al., J. Mol. Biol., 196:901 (1987), the contents of which are incorporated herein by reference herein in their entirety). Another method uses a particular framework derived from the consensus sequence of all human antibodies of a particular subgroup of light or heavy chains. The same framework may be used for several different humanized antibodies (see, e.g., Nicholson et al. Mol. Immun. 34 (16-17): 1157-1165 (1997); Carter et al., Proc. Natl. Acad. Sci. USA, 89:4285 (1992); Presta et al., J. Immunol., 151:2623 (1993), the contents of which are incorporated herein by reference herein in their entirety). In some embodiments, the framework region, e.g., all four framework regions, of the heavy chain variable region are derived from a VH4_4-59 germline sequence. In one embodiment, the framework region can comprise, one, two, three, four or five modifications, e.g., substitutions, e.g., from the amino acid at the corresponding murine sequence. In one embodiment, the framework region, e.g., all four framework regions of the light chain variable region are derived from a VK3_1.25 germline sequence. In one embodiment, the framework region can comprise, one, two, three, four or five modifications, e.g., substitutions, e.g., from the amino acid at the corresponding murine sequence.

In some aspects, the portion of a CAR composition of the invention that comprises an antibody fragment is humanized with retention of high affinity for the target antigen and other favorable biological properties. According to one aspect of the invention, humanized antibodies and antibody fragments are prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, e.g., the analysis of residues that influence the ability of the candidate immunoglobulin to bind the target antigen. In this way, FR residues can be selected and combined from the recipient and import sequences so that the desired antibody or antibody fragment characteristic, such as increased affinity for the target antigen, is achieved. In general, the CDR residues are directly and most substantially involved in influencing antigen binding.

A humanized antibody or antibody fragment may retain a similar antigenic specificity as the original antibody, e.g., in the present invention, the ability to bind human a cancer associated antigen as described herein. In some embodiments, a humanized antibody or antibody fragment may have improved affinity and/or specificity of binding to human a cancer associated antigen as described herein.

In one aspect, the antigen binding domain of the invention is characterized by particular functional features or properties of an antibody or antibody fragment. For example, in one aspect, the portion of a CAR composition of the invention that comprises an antigen binding domain specifically binds a tumor antigen as described herein.

In one aspect, the anti-cancer associated antigen as described herein binding domain is a fragment, e.g., a single chain variable fragment (scFv). In one aspect, the anti-cancer associated antigen as described herein binding domain is a Fv, a Fab, a (Fab′)2, or a bi-functional (e.g. bi-specific) hybrid antibody (e.g., Lanzavecchia et al., Eur. J. Immunol. 17, 105 (1987)). In one aspect, the antibodies and fragments thereof of the invention binds a cancer associated antigen as described herein protein with wild-type or enhanced affinity.

In some instances, scFvs can be prepared according to method known in the art (see, for example, Bird et al., (1988) Science 242:423-426 and Huston et al., (1988) Proc. Natl. Acad. Sci. USA 85:5879-5883). ScFv molecules can be produced by linking VH and VL regions together using flexible polypeptide linkers. The scFv molecules comprise a linker (e.g., a Ser-Gly linker) with an optimized length and/or amino acid composition. The linker length can greatly affect how the variable regions of a scFv fold and interact. In fact, if a short polypeptide linker is employed (e.g., between 5-10 amino acids) intrachain folding is prevented. Interchain folding is also required to bring the two variable regions together to form a functional epitope binding site. For examples of linker orientation and size see, e.g., Hollinger et al. 1993 Proc Natl Acad. Sci. U.S.A. 90:6444-6448, U.S. Patent Application Publication Nos. 2005/0100543, 2005/0175606, 2007/0014794, and PCT publication Nos. WO2006/020258 and WO2007/024715, is incorporated herein by reference.

An scFv can comprise a linker of at least 1, 2, 3, 4, 5, 6, 7, 8, 9, 10, 11, 12, 13, 14, 15, 16, 17, 18, 19, 20, 25, 30, 35, 40, 45, 50, or more amino acid residues between its VL and VH regions. The linker sequence may comprise any naturally occurring amino acid. In some embodiments, the linker sequence comprises amino acids glycine and serine. In another embodiment, the linker sequence comprises sets of glycine and serine repeats such as (Gly4Ser)n, where n is a positive integer equal to or greater than 1 (SEQ ID NO:22). In one embodiment, the linker can be (Gly4Ser)₄ (SEQ ID NO:29) or (Gly4Ser)₃(SEQ ID NO:30). Variation in the linker length may retain or enhance activity, giving rise to superior efficacy in activity studies.

In another aspect, the antigen binding domain is a T cell receptor (“TCR”), or a fragment thereof, for example, a single chain TCR (scTCR). Methods to make such TCRs are known in the art. See, e.g., Willemsen R A et al, Gene Therapy 7: 1369-1377 (2000); Zhang T et al, Cancer Gene Ther 11: 487-496 (2004); Aggen et al, Gene Ther. 19(4):365-74 (2012) (references are incorporated herein by its entirety). For example, scTCR can be engineered that contains the Vα and Vβ genes from a T cell clone linked by a linker (e.g., a flexible peptide). This approach is very useful to cancer associated target that itself is intracellar, however, a fragment of such antigen (peptide) is presented on the surface of the cancer cells by MHC.

In one embodiment, an antigen binding domain against EGFRvIII is an antigen binding portion, e.g., CDRs, of a CAR, antibody or antigen-binding fragment thereof described in, e.g., PCT publication WO2014/130657 or US2014/0322275A1. In one embodiment, the CAR molecule comprises an EGFRvIII CAR, or an antigen binding domain according to Table 2 or SEQ ID NO:11 of WO 2014/130657, incorporated herein by reference, or a sequence substantially identical thereto (e.g., at least 85%, 90%, 95% or more identical thereto). The amino acid and nucleotide sequences encoding the EGFRvIII CAR molecules and antigen binding domains (e.g., including one, two, three VH CDRs; and one, two, three VL CDRs according to Kabat or Chothia), are specified in WO 2014/130657.

In one embodiment, an antigen binding domain against mesothelin is an antigen binding portion, e.g., CDRs, of an antibody, antigen-binding fragment or CAR described in, e.g., PCT publication WO2015/090230. In one embodiment, an antigen binding domain against mesothelin is an antigen binding portion, e.g., CDRs, of an antibody, antigen-binding fragment, or CAR described in, e.g., PCT publication WO1997/025068, WO1999/028471, WO2005/014652, WO2006/099141, WO2009/045957, WO2009/068204, WO2013/142034, WO2013/040557, or WO2013/063419.

In an embodiment, the CAR molecule comprises a mesothelin CAR described herein, e.g., a mesothelin CAR described in WO 2015/090230, incorporated herein by reference. In some embodiments, the mesothelin CAR comprises an amino acid, or has a nucleotide sequence shown in WO 2015/090230 incorporated herein by reference, or a sequence substantially identical to any of the aforesaid sequences (e.g., at least 85%, 90%, 95% or more identical to any of the aforesaid mesothelin CAR sequences). In one embodiment, the CAR molecule comprises a mesothelin CAR, or an antigen binding domain according to Tables 2-3 of WO 2015/090230, incorporated herein by reference, or a sequence substantially identical thereto (e.g., at least 85%, 90%, 95% or more identical thereto). The amino acid and nucleotide sequences encoding the mesothelin CAR molecules and antigen binding domains (e.g., including one, two, three VH CDRs; and one, two, three VL CDRs according to Kabat or Chothia), are specified in WO 2015/090230.

In one embodiment, an antigen binding domain against CD123 is an antigen binding portion, e.g., CDRs, of an antibody, antigen-binding fragment or CAR described in, e.g., PCT publication WO2016/028896. In one embodiment, an antigen binding domain against CD123 is an antigen binding portion, e.g., CDRs, of an antibody, antigen-binding fragment or CAR described in, e.g., PCT publication WO2014/130635. In one embodiment, an antigen binding domain against CD123 is an antigen binding portion, e.g., CDRs, of an antibody, antigen-binding fragment, or CAR described in, e.g., PCT publication WO2014/138805, WO2014/138819, WO2013/173820, WO2014/144622, WO2001/66139, WO2010/126066, WO2014/144622, or US2009/0252742.

In one embodiment, an antigen binding domain against CD123 is an antigen binding portion, e.g., CDRs, of an antibody, antigen-binding fragment or CAR described in, e.g., US2014/0322212A1 or US2016/0068601A1, both incorporated herein by reference. In some embodiments, the CD123 CAR comprises an amino acid, or has a nucleotide sequence shown in US2014/0322212A1 or US2016/0068601A1, both incorporated herein by reference, or a sequence substantially identical to any of the aforesaid sequences (e.g., at least 85%, 90%, 95% or more identical to any of the aforesaid CD123 CAR sequences). In one embodiment, the CAR molecule comprises a CD123 CAR (e.g., any of the CAR1-CARE), or an antigen binding domain according to Tables 1-2 of WO 2014/130635, incorporated herein by reference, or a sequence substantially identical thereto (e.g., at least 85%, 90%, 95% or more identical to any of the aforesaid CD123 CAR sequences). The amino acid and nucleotide sequences encoding the CD123 CAR molecules and antigen binding domains (e.g., including one, two, three VH CDRs; and one, two, three VL CDRs according to Kabat or Chothia), are specified in WO 2014/130635.

In other embodiments, the CAR molecule comprises a CD123 CAR comprises a CAR molecule (e.g., any of the CAR123-1 to CAR123-4 and hzCAR123-1 to hzCAR123-32), or an antigen binding domain according to Tables 2, 6, and 9 of WO2016/028896, incorporated herein by reference, or a sequence substantially identical thereto (e.g., at least 85%, 90%, 95% or more identical to any of the aforesaid CD123 CAR sequences). The amino acid and nucleotide sequences encoding the CD123 CAR molecules and antigen binding domains (e.g., including one, two, three VH CDRs; and one, two, three VL CDRs according to Kabat or Chothia), are specified in WO2016/028896.

In one embodiment, an antigen binding domain against CD22 is an antigen binding portion, e.g., CDRs, of an antibody described in, e.g., Haso et al., Blood, 121(7): 1165-1174 (2013); Wayne et al., Clin Cancer Res 16(6): 1894-1903 (2010); Kato et al., Leuk Res 37(1):83-88 (2013); Creative BioMart (creativebiomart.net): MOM-18047-S(P).

In one embodiment, an antigen binding domain against CS-1 is an antigen binding portion, e.g., CDRs, of Elotuzumab (BMS), see e.g., Tai et al., 2008, Blood 112(4):1329-37; Tai et al., 2007, Blood. 110(5):1656-63.

In one embodiment, an antigen binding domain against CLL-1 is an antigen binding portion, e.g., CDRs, of an antibody available from R&D, ebiosciences, Abcam, for example, PE-CLL1-hu Cat #353604 (BioLegend); and PE-CLL1 (CLEC12A) Cat #562566 (BD).

In other embodiments, the CLL1 CAR includes a CAR molecule, or an antigen binding domain according to Table 2 of WO2016/014535, incorporated herein by reference. The amino acid and nucleotide sequences encoding the CLL-1 CAR molecules and antigen binding domains (e.g., including one, two, three VH CDRs; and one, two, three VL CDRs according to Kabat or Chothia), are specified in WO2016/014535.

In one embodiment, an antigen binding domain against CD33 is an antigen binding portion, e.g., CDRs, of an antibody described in, e.g., Bross et al., Clin Cancer Res 7(6):1490-1496 (2001) (Gemtuzumab Ozogamicin, hP67.6), Caron et al., Cancer Res 52(24):6761-6767 (1992) (Lintuzumab, HuM195), Lapusan et al., Invest New Drugs 30(3):1121-1131 (2012) (AVE9633), Aigner et al., Leukemia 27(5): 1107-1115 (2013) (AMG330, CD33 BiTE), Dutour et al., Adv hematol 2012:683065 (2012), and Pizzitola et al., Leukemia doi:10.1038/Lue.2014.62 (2014).

In one embodiment, an antigen binding domain against CD33 is an antigen binding portion, e.g., CDRs, of an antibody described in, US2016/0096892A1, incorporated herein by reference. In some embodiments, the CD33 CAR comprises an amino acid, or has a nucleotide sequence shown in US2016/0096892A1, incorporated herein by reference, or a sequence substantially identical to any of the aforesaid sequences (e.g., at least 85%, 90%, 95% or more identical to any of the aforesaid CD33 CAR sequences). In other embodiments, the CD33 CAR CAR or antigen binding domain thereof can include a CAR molecule (e.g., any of CAR33-1 to CAR-33-9), or an antigen binding domain according to Table 2 or 9 of WO2016/014576, incorporated herein by reference, or a sequence substantially identical to any of the aforesaid sequences (e.g., at least 85%, 90%, 95% or more identical to any of the aforesaid CD33 CAR sequences). The amino acid and nucleotide sequences encoding the CD33 CAR molecules and antigen binding domains (e.g., including one, two, three VH CDRs; and one, two, three VL CDRs according to Kabat or Chothia), are specified in WO2016/014576.

In one embodiment, an antigen binding domain against GD2 is an antigen binding portion, e.g., CDRs, of an antibody described in, e.g., Mujoo et al., Cancer Res. 47(4):1098-1104 (1987); Cheung et al., Cancer Res 45(6):2642-2649 (1985), Cheung et al., J Clin Oncol 5(9):1430-1440 (1987), Cheung et al., J Clin Oncol 16(9):3053-3060 (1998), Handgretinger et al., Cancer Immunol Immunother 35(3):199-204 (1992). In some embodiments, an antigen binding domain against GD2 is an antigen binding portion of an antibody selected from mAb 14.18, 14G2a, ch14.18, hu14.18, 3F8, hu3F8, 3G6, 8B6, 60C3, 10B8, ME36.1, and 8H9, see e.g., WO2012033885, WO2013040371, WO2013192294, WO2013061273, WO2013123061, WO2013074916, and WO201385552. In some embodiments, an antigen binding domain against GD2 is an antigen binding portion of an antibody described in US Publication No.: 20100150910 or PCT Publication No.: WO 2011160119.

In one embodiment, an antigen binding domain against BCMA is an antigen binding portion, e.g., CDRs, of an antibody, antigen-binding fragment or CAR described in, e.g., PCT publication WO2016/014565, e.g., the antigen binding portion of CAR BCMA-10 as described in WO2016/014565. In one embodiment, an antigen binding domain against BCMA is an antigen binding portion, e.g., CDRs, of an antibody, antigen-binding fragment or CAR described in, e.g., PCT publication WO2016/014789. In one embodiment, an antigen binding domain against BCMA is an antigen binding portion, e.g., CDRs, of an antibody described in, e.g., WO2012/163805, WO2001/12812, and WO2003/062401.

In other embodiment, the CAR molecule comprises a BCMA CAR molecule, or an antigen binding domain against BCMA described herein, e.g., a BCMA CAR described in US-2016-0046724-A1 or WO2016/014565. In some embodiments, the BCMA CAR comprises an amino acid, or has a nucleotide sequence of a CAR molecule, or an antigen binding domain according to US-2016-0046724-A1, or Table 1 or 16, SEQ ID NO: 271 or SEQ ID NO: 273 of WO2016/014565, incorporated herein by reference, or a sequence substantially identical to any of the aforesaid sequences (e.g., at least 85%, 90%, 95% or more identical to any of the aforesaid BCMA CAR sequences). The amino acid and nucleotide sequences encoding the BCMA CAR molecules and antigen binding domains (e.g., including one, two, three VH CDRs; and one, two, three VL CDRs according to Kabat or Chothia), are specified in WO2016/014565.

In one embodiment, an antigen binding domain against GFR ALPHA-4 CAR antigen is an antigen binding portion, e.g., CDRs, of an antibody described in, e.g., WO2016/025880, incorporated herein by reference. In one embodiment, the CAR molecule comprises an a GFR ALPHA-4 CAR, e.g., a CAR molecule, or an antigen binding domain according to Table 2 of WO2016/025880, incorporated herein by reference, or a sequence substantially identical to any of the aforesaid sequences (e.g., at least 85%, 90%, 95% or more identical to any of the aforesaid GFR ALPHA-4 sequences). The amino acid and nucleotide sequences encoding the GFR ALPHA-4 CAR molecules and antigen binding domains (e.g., including one, two, three VH CDRs; and one, two, three VL CDRs according to Kabat or Chothia), are specified in WO2016/025880.

In one embodiment, an antigen binding domain against Tn antigen is an antigen binding portion, e.g., CDRs, of an antibody described in, e.g., U.S. Pat. No. 8,440,798; Brooks et al., PNAS 107(22):10056-10061 (2010), and Stone et al., Oncolmmunology 1(6):863-873(2012).

In one embodiment, an antigen binding domain against PSMA is an antigen binding portion, e.g., CDRs, of an antibody described in, e.g., Parker et al., Protein Expr Purif 89(2):136-145 (2013), US 20110268656 (J591 ScFv); Frigerio et al, European J Cancer 49(9):2223-2232 (2013) (scFvD2B); WO 2006125481 (mAbs 3/A12, 3/E7 and 3/F11) and single chain antibody fragments (scFv A5 and D7).

In one embodiment, an antigen binding domain against ROR1 is an antigen binding portion, e.g., CDRs, of an antibody described in, e.g., Hudecek et al., Clin Cancer Res 19(12):3153-3164 (2013); WO 2011159847; and US20130101607.

In one embodiment, an antigen binding domain against FLT3 is an antigen binding portion, e.g., CDRs, of an antibody described in, e.g., WO2011076922, U.S. Pat. No. 5,777,084, EP0754230, US20090297529, and several commercial catalog antibodies (R&D, ebiosciences, Abcam).

In one embodiment, an antigen binding domain against TAG72 is an antigen binding portion, e.g., CDRs, of an antibody described in, e.g., Hombach et al., Gastroenterology 113(4):1163-1170 (1997); and Abcam ab691.

In one embodiment, an antigen binding domain against FAP is an antigen binding portion, e.g., CDRs, of an antibody described in, e.g., Ostermann et al., Clinical Cancer Research 14:4584-4592 (2008) (FAPS), US Pat. Publication No. 2009/0304718; sibrotuzumab (see e.g., Hofheinz et al., Oncology Research and Treatment 26(1), 2003); and Tran et al., J Exp Med 210(6):1125-1135 (2013).

In one embodiment, an antigen binding domain against CD38 is an antigen binding portion, e.g., CDRs, of daratumumab (see, e.g., Groen et al., Blood 116(21):1261-1262 (2010); MOR202 (see, e.g., U.S. Pat. No. 8,263,746); or antibodies described in U.S. Pat. No. 8,362,211.

In one embodiment, an antigen binding domain against CD44v6 is an antigen binding portion, e.g., CDRs, of an antibody described in, e.g., Casucci et al., Blood 122(20):3461-3472 (2013).

In one embodiment, an antigen binding domain against CEA is an antigen binding portion, e.g., CDRs, of an antibody described in, e.g., Chmielewski et al., Gastoenterology 143(4):1095-1107 (2012).

In one embodiment, an antigen binding domain against EPCAM is an antigen binding portion, e.g., CDRS, of an antibody selected from MT110, EpCAM-CD3 bispecific Ab (see, e.g., clinicaltrials.gov/ct2/show/NCT00635596); Edrecolomab; 3622W94; ING-1; and adecatumumab (MT201).

In one embodiment, an antigen binding domain against PRSS21 is an antigen binding portion, e.g., CDRs, of an antibody described in U.S. Pat. No. 8,080,650.

In one embodiment, an antigen binding domain against B7H3 is an antigen binding portion, e.g., CDRs, of an antibody MGA271 (Macrogenics).

In one embodiment, an antigen binding domain against KIT is an antigen binding portion, e.g., CDRs, of an antibody described in, e.g., U.S. Pat. No. 7,915,391, US20120288506, and several commercial catalog antibodies.

In one embodiment, an antigen binding domain against IL-13Ra2 is an antigen binding portion, e.g., CDRs, of an antibody described in, e.g., WO2008/146911, WO2004087758, several commercial catalog antibodies, and WO2004087758.

In one embodiment, an antigen binding domain against CD30 is an antigen binding portion, e.g., CDRs, of an antibody described in, e.g., U.S. Pat. No. 7,090,843 B1, and EP0805871.

In one embodiment, an antigen binding domain against GD3 is an antigen binding portion, e.g., CDRs, of an antibody described in, e.g., U.S. Pat. Nos. 7,253,263; 8,207,308; US 20120276046; EP1013761; WO2005035577; and U.S. Pat. No. 6,437,098.

In one embodiment, an antigen binding domain against CD171 is an antigen binding portion, e.g., CDRs, of an antibody described in, e.g., Hong et al., J Immunother 37(2):93-104 (2014).

In one embodiment, an antigen binding domain against IL-11Ra is an antigen binding portion, e.g., CDRs, of an antibody available from Abcam (cat # ab55262) or Novus Biologicals (cat # EPR5446). In another embodiment, an antigen binding domain again IL-11Ra is a peptide, see, e.g., Huang et al., Cancer Res 72(1):271-281 (2012).

In one embodiment, an antigen binding domain against PSCA is an antigen binding portion, e.g., CDRs, of an antibody described in, e.g., Morgenroth et al., Prostate 67(10):1121-1131 (2007) (scFv 7F5); Nejatollahi et al., J of Oncology 2013(2013), article ID 839831 (scFv C5-II); and US Pat Publication No. 20090311181.

In one embodiment, an antigen binding domain against VEGFR2 is an antigen binding portion, e.g., CDRs, of an antibody described in, e.g., Chinnasamy et al., J Clin Invest 120(11):3953-3968 (2010).

In one embodiment, an antigen binding domain against LewisY is an antigen binding portion, e.g., CDRs, of an antibody described in, e.g., Kelly et al., Cancer Biother Radiopharm 23(4):411-423 (2008) (hu3S193 Ab (scFvs)); Dolezal et al., Protein Engineering 16(1):47-56 (2003) (NC10 scFv).

In one embodiment, an antigen binding domain against CD24 is an antigen binding portion, e.g., CDRs, of an antibody described in, e.g., Maliar et al., Gastroenterology 143(5):1375-1384 (2012).

In one embodiment, an antigen binding domain against PDGFR-beta is an antigen binding portion, e.g., CDRs, of an antibody Abcam ab32570.

In one embodiment, an antigen binding domain against SSEA-4 is an antigen binding portion, e.g., CDRs, of antibody MC813 (Cell Signaling), or other commercially available antibodies.

In one embodiment, an antigen binding domain against CD20 is an antigen binding portion, e.g., CDRs, of the antibody Rituximab, Ofatumumab, Ocrelizumab, Veltuzumab, or GA101.

In one embodiment, an antigen binding domain against Folate receptor alpha is an antigen binding portion, e.g., CDRs, of the antibody IMGN853, or an antibody described in US20120009181; U.S. Pat. No. 4,851,332, LK26: U.S. Pat. No. 5,952,484.

In one embodiment, an antigen binding domain against ERBB2 (Her2/neu) is an antigen binding portion, e.g., CDRs, of the antibody trastuzumab, or pertuzumab.

In one embodiment, an antigen binding domain against MUC1 is an antigen binding portion, e.g., CDRs, of the antibody SAR566658.

In one embodiment, the antigen binding domain against EGFR is antigen binding portion, e.g., CDRs, of the antibody cetuximab, panitumumab, zalutumumab, nimotuzumab, or matuzumab.

In one embodiment, an antigen binding domain against NCAM is an antigen binding portion, e.g., CDRs, of the antibody clone 2-2B: MAB5324 (EMD Millipore).

In one embodiment, an antigen binding domain against Ephrin B2 is an antigen binding portion, e.g., CDRs, of an antibody described in, e.g., Abengozar et al., Blood 119(19):4565-4576 (2012).

In one embodiment, an antigen binding domain against IGF-I receptor is an antigen binding portion, e.g., CDRs, of an antibody described in, e.g., U.S. Pat. No. 8,344,112 B2; EP2322550 A1; WO 2006/138315, or PCT/US2006/022995.

In one embodiment, an antigen binding domain against CAIX is an antigen binding portion, e.g., CDRs, of the antibody clone 303123 (R&D Systems).

In one embodiment, an antigen binding domain against LMP2 is an antigen binding portion, e.g., CDRs, of an antibody described in, e.g., U.S. Pat. No. 7,410,640, or US20050129701.

In one embodiment, an antigen binding domain against gp100 is an antigen binding portion, e.g., CDRs, of the antibody HMB45, NKIbetaB, or an antibody described in WO2013165940, or US20130295007

In one embodiment, an antigen binding domain against tyrosinase is an antigen binding portion, e.g., CDRs, of an antibody described in, e.g., U.S. Pat. No. 5,843,674; or US19950504048.

In one embodiment, an antigen binding domain against EphA2 is an antigen binding portion, e.g., CDRs, of an antibody described in, e.g., Yu et al., Mol Ther 22(1):102-111 (2014).

In one embodiment, an antigen binding domain against GD3 is an antigen binding portion, e.g., CDRs, of an antibody described in, e.g., U.S. Pat. Nos. 7,253,263; 8,207,308; US 20120276046; EP1013761 A3; 20120276046; WO2005035577; or U.S. Pat. No. 6,437,098.

In one embodiment, an antigen binding domain against fucosyl GM1 is an antigen binding portion, e.g., CDRs, of an antibody described in, e.g., US20100297138; or WO2007/067992.

In one embodiment, an antigen binding domain against sLe is an antigen binding portion, e.g., CDRs, of the antibody G193 (for lewis Y), see Scott A M et al, Cancer Res 60: 3254-61 (2000), also as described in Neeson et al, J Immunol May 2013 190 (Meeting Abstract Supplement) 177.10.

In one embodiment, an antigen binding domain against GM3 is an antigen binding portion, e.g., CDRs, of the antibody CA 2523449 (mAb 14F7).

In one embodiment, an antigen binding domain against HMWMAA is an antigen binding portion, e.g., CDRs, of an antibody described in, e.g., Kmiecik et al., Oncoimmunology 3(1):e27185 (2014) (PMID: 24575382) (mAb9.2.27); U.S. Pat. No. 6,528,481; WO2010033866; or US 20140004124.

In one embodiment, an antigen binding domain against o-acetyl-GD2 is an antigen binding portion, e.g., CDRs, of the antibody 8B6.

In one embodiment, an antigen binding domain against TEM1/CD248 is an antigen binding portion, e.g., CDRs, of an antibody described in, e.g., Marty et al., Cancer Lett 235(2):298-308 (2006); Zhao et al., J Immunol Methods 363(2):221-232 (2011).

In one embodiment, an antigen binding domain against CLDN6 is an antigen binding portion, e.g., CDRs, of the antibody IMAB027 (Ganymed Pharmaceuticals), see e.g., clinicaltrial.gov/show/NCT02054351.

In one embodiment, an antigen binding domain against TSHR is an antigen binding portion, e.g., CDRs, of an antibody described in, e.g., U.S. Pat. Nos. 8,603,466; 8,501,415; or U.S. Pat. No. 8,309,693.

In one embodiment, an antigen binding domain against GPRC5D is an antigen binding portion, e.g., CDRs, of the antibody FAB6300A (R&D Systems); or LS-A4180 (Lifespan Biosciences).

In one embodiment, an antigen binding domain against CD97 is an antigen binding portion, e.g., CDRs, of an antibody described in, e.g., U.S. Pat. No. 6,846,911; de Groot et al., J Immunol 183(6):4127-4134 (2009); or an antibody from R&D:MAB3734.

In one embodiment, an antigen binding domain against ALK is an antigen binding portion, e.g., CDRs, of an antibody described in, e.g., Mino-Kenudson et al., Clin Cancer Res 16(5):1561-1571 (2010).

In one embodiment, an antigen binding domain against polysialic acid is an antigen binding portion, e.g., CDRs, of an antibody described in, e.g., Nagae et al., J Biol Chem 288(47):33784-33796 (2013).

In one embodiment, an antigen binding domain against PLAC1 is an antigen binding portion, e.g., CDRs, of an antibody described in, e.g., Ghods et al., Biotechnol Appl Biochem 2013 doi:10.1002/bab.1177.

In one embodiment, an antigen binding domain against GloboH is an antigen binding portion of the antibody VK9; or an antibody described in, e.g., Kudryashov V et al, Glycoconj J.15(3):243-9 (1998), Lou et al., Proc Natl Acad Sci USA 111(7):2482-2487 (2014); MBr1: Bremer E-G et al. J Biol Chem 259:14773-14777 (1984).

In one embodiment, an antigen binding domain against NY-BR-1 is an antigen binding portion, e.g., CDRs of an antibody described in, e.g., Jager et al., Appl Immunohistochem Mol Morphol 15(1):77-83 (2007).

In one embodiment, an antigen binding domain against WT-1 is an antigen binding portion, e.g., CDRs, of an antibody described in, e.g., Dao et al., Sci Transl Med 5(176):176ra33 (2013); or WO2012/135854.

In one embodiment, an antigen binding domain against MAGE-A1 is an antigen binding portion, e.g., CDRs, of an antibody described in, e.g., Willemsen et al., J Immunol 174(12):7853-7858 (2005) (TCR-like scFv).

In one embodiment, an antigen binding domain against sperm protein 17 is an antigen binding portion, e.g., CDRs, of an antibody described in, e.g., Song et al., Target Oncol 2013 Aug. 14 (PMID: 23943313); Song et al., Med Oncol 29(4):2923-2931 (2012).

In one embodiment, an antigen binding domain against Tie 2 is an antigen binding portion, e.g., CDRs, of the antibody AB33 (Cell Signaling Technology).

In one embodiment, an antigen binding domain against MAD-CT-2 is an antigen binding portion, e.g., CDRs, of an antibody described in, e.g., PMID: 2450952; U.S. Pat. No. 7,635,753.

In one embodiment, an antigen binding domain against Fos-related antigen 1 is an antigen binding portion, e.g., CDRs, of the antibody 12F9 (Novus Biologicals).

In one embodiment, an antigen binding domain against MelanA/MART1 is an antigen binding portion, e.g., CDRs, of an antibody described in, EP2514766 A2; or U.S. Pat. No. 7,749,719.

In one embodiment, an antigen binding domain against sarcoma translocation breakpoints is an antigen binding portion, e.g., CDRs, of an antibody described in, e.g., Luo et al, EMBO Mol. Med. 4(6):453-461 (2012).

In one embodiment, an antigen binding domain against TRP-2 is an antigen binding portion, e.g., CDRs, of an antibody described in, e.g., Wang et al, J Exp Med. 184(6):2207-16 (1996).

In one embodiment, an antigen binding domain against CYP1B1 is an antigen binding portion, e.g., CDRs, of an antibody described in, e.g., Maecker et al, Blood 102 (9): 3287-3294 (2003).

In one embodiment, an antigen binding domain against RAGE-1 is an antigen binding portion, e.g., CDRs, of the antibody MAB5328 (EMD Millipore).

In one embodiment, an antigen binding domain against human telomerase reverse transcriptase is an antigen binding portion, e.g., CDRs, of the antibody cat no: LS-B95-100 (Lifespan Biosciences)

In one embodiment, an antigen binding domain against intestinal carboxyl esterase is an antigen binding portion, e.g., CDRs, of the antibody 4F12: cat no: LS-B6190-50 (Lifespan Biosciences).

In one embodiment, an antigen binding domain against mut hsp70-2 is an antigen binding portion, e.g., CDRs, of the antibody Lifespan Biosciences: monoclonal: cat no: LS-C133261-100 (Lifespan Biosciences).

In one embodiment, an antigen binding domain against CD79a is an antigen binding portion, e.g., CDRs, of the antibody Anti-CD79a antibody [HM47/A9] (ab3121), available from Abcam; antibody CD79A Antibody #3351 available from Cell Signalling Technology; or antibody HPA017748-Anti-CD79A antibody produced in rabbit, available from Sigma Aldrich.

In one embodiment, an antigen binding domain against CD79b is an antigen binding portion, e.g., CDRs, of the antibody polatuzumab vedotin, anti-CD79b described in Dornan et al., “Therapeutic potential of an anti-CD79b antibody-drug conjugate, anti-CD79b-vc-MMAE, for the treatment of non-Hodgkin lymphoma” Blood. 2009 Sep. 24; 114(13):2721-9. doi:

10.1182/blood-2009-02-205500. Epub 2009 Jul. 24, or the bispecific antibody Anti-CD79b/CD3 described in “4507 Pre-Clinical Characterization of T Cell-Dependent Bispecific Antibody Anti-CD79b/CD3 As a Potential Therapy for B Cell Malignancies” Abstracts of 56^{th} ASH Annual Meeting and Exposition, San Francisco, Calif. Dec. 6-9 2014.

In one embodiment, an antigen binding domain against CD72 is an antigen binding portion, e.g., CDRs, of the antibody J3-109 described in Myers, and Uckun, “An anti-CD72 immunotoxin against therapy-refractory B-lineage acute lymphoblastic leukemia.” Leuk Lymphoma. 1995 June; 18(1-2):119-22, or anti-CD72 (10D6.8.1, mIgG1) described in Polson et al., “Antibody-Drug Conjugates for the Treatment of Non-Hodgkin's Lymphoma: Target and Linker-Drug Selection” Cancer Res Mar. 15, 2009 69; 2358.

In one embodiment, an antigen binding domain against LAIR1 is an antigen binding portion, e.g., CDRs, of the antibody ANT-301 LAIR1 antibody, available from ProSpec; or anti-human CD305 (LAIR1) Antibody, available from BioLegend.

In one embodiment, an antigen binding domain against FCAR is an antigen binding portion, e.g., CDRs, of the antibody CD89/FCARAntibody (Catalog #10414-H08H), available from Sino Biological Inc.

In one embodiment, an antigen binding domain against LILRA2 is an antigen binding portion, e.g., CDRs, of the antibody LILRA2 monoclonal antibody (M17), clone 3C7, available from Abnova, or Mouse Anti-LILRA2 antibody, Monoclonal (2D7), available from Lifespan Biosciences.

In one embodiment, an antigen binding domain against CD300LF is an antigen binding portion, e.g., CDRs, of the antibody Mouse Anti-CMRF35-like molecule 1 antibody, Monoclonal[UP-D2], available from BioLegend, or Rat Anti-CMRF35-like molecule 1 antibody, Monoclonal[234903], available from R&D Systems.

In one embodiment, an antigen binding domain against CLEC12A is an antigen binding portion, e.g., CDRs, of the antibody Bispecific T cell Engager (BiTE) scFv-antibody and ADC described in Noordhuis et al., “Targeting of CLEC12A In Acute Myeloid Leukemia by Antibody-Drug-Conjugates and Bispecific CLL-1×CD3 BiTE Antibody” 53^{rd} ASH Annual Meeting and Exposition, Dec. 10-13, 2011, and MCLA-117 (Merus).

In one embodiment, an antigen binding domain against BST2 (also called CD317) is an antigen binding portion, e.g., CDRs, of the antibody Mouse Anti-CD317 antibody, Monoclonal[3H4], available from Antibodies-Online or Mouse Anti-CD317 antibody, Monoclonal[696739], available from R&D Systems.

In one embodiment, an antigen binding domain against EMR2 (also called CD312) is an antigen binding portion, e.g., CDRs, of the antibody Mouse Anti-CD312 antibody, Monoclonal[LS-B8033] available from Lifespan Biosciences, or Mouse Anti-CD312 antibody, Monoclonal[494025] available from R&D Systems.

In one embodiment, an antigen binding domain against LY75 is an antigen binding portion, e.g., CDRs, of the antibody Mouse Anti-Lymphocyte antigen 75 antibody, Monoclonal[HD30] available from EMD Millipore or Mouse Anti-Lymphocyte antigen 75 antibody, Monoclonal[A15797] available from Life Technologies.

In one embodiment, an antigen binding domain against GPC3 is an antigen binding portion, e.g., CDRs, of the antibody hGC33 described in Nakano K, Ishiguro T, Konishi H, et al. Generation of a humanized anti-glypican 3 antibody by CDR grafting and stability optimization. Anticancer Drugs. 2010 November; 21(10):907-916, or MDX-1414, HN3, or YP7, all three of which are described in Feng et al., “Glypican-3 antibodies: a new therapeutic target for liver cancer.” FEBS Lett. 2014 Jan. 21; 588(2):377-82.

In one embodiment, an antigen binding domain against FCRL5 is an antigen binding portion, e.g., CDRs, of the anti-FcRL5 antibody described in Elkins et al., “FcRL5 as a target of antibody-drug conjugates for the treatment of multiple myeloma” Mol Cancer Ther. 2012 October; 11(10):2222-32.

In one embodiment, an antigen binding domain against IGLL1 is an antigen binding portion, e.g., CDRs, of the antibody Mouse Anti-Immunoglobulin lambda-like polypeptide 1 antibody, Monoclonal[AT1G4] available from Lifespan Biosciences, Mouse Anti-Immunoglobulin lambda-like polypeptide 1 antibody, Monoclonal[HSL11] available from BioLegend.

In one embodiment, the antigen binding domain comprises one, two three (e.g., all three) heavy chain CDRs, HC CDR1, HC CDR2 and HC CDR3, from an antibody listed above, and/or one, two, three (e.g., all three) light chain CDRs, LC CDR1, LC CDR2 and LC CDR3, from an antibody listed above. In one embodiment, the antigen binding domain comprises a heavy chain variable region and/or a variable light chain region of an antibody listed above.

In another aspect, the antigen binding domain comprises a humanized antibody or an antibody fragment. In some aspects, a non-human antibody is humanized, where specific sequences or regions of the antibody are modified to increase similarity to an antibody naturally produced in a human or fragment thereof. In one aspect, the antigen binding domain is humanized.

Bispecific CARs

In an embodiment a multispecific antibody molecule is a bispecific antibody molecule. A bispecific antibody has specificity for no more than two antigens. A bispecific antibody molecule is characterized by a first immunoglobulin variable domain sequence which has binding specificity for a first epitope and a second immunoglobulin variable domain sequence that has binding specificity for a second epitope. In an embodiment the first and second epitopes are on the same antigen, e.g., the same protein (or subunit of a multimeric protein). In an embodiment the first and second epitopes overlap. In an embodiment the first and second epitopes do not overlap. In an embodiment the first and second epitopes are on different antigens, e.g., different proteins (or different subunits of a multimeric protein). In an embodiment a bispecific antibody molecule comprises a heavy chain variable domain sequence and a light chain variable domain sequence which have binding specificity for a first epitope and a heavy chain variable domain sequence and a light chain variable domain sequence which have binding specificity for a second epitope. In an embodiment a bispecific antibody molecule comprises a half antibody having binding specificity for a first epitope and a half antibody having binding specificity for a second epitope. In an embodiment a bispecific antibody molecule comprises a half antibody, or fragment thereof, having binding specificity for a first epitope and a half antibody, or fragment thereof, having binding specificity for a second epitope. In an embodiment a bispecific antibody molecule comprises a scFv, or fragment thereof, have binding specificity for a first epitope and a scFv, or fragment thereof, have binding specificity for a second epitope.

In certain embodiments, the antibody molecule is a multi-specific (e.g., a bispecific or a trispecific) antibody molecule. Protocols for generating bispecific or heterodimeric antibody molecules are known in the art; including but not limited to, for example, the “knob in a hole” approach described in, e.g., U.S. Pat. No. 5,731,168; the electrostatic steering Fc pairing as described in, e.g., WO 09/089004, WO 06/106905 and WO 2010/129304; Strand Exchange Engineered Domains (SEED) heterodimer formation as described in, e.g., WO 07/110205; Fab arm exchange as described in, e.g., WO 08/119353, WO 2011/131746, and WO 2013/060867; double antibody conjugate, e.g., by antibody cross-linking to generate a bi-specific structure using a heterobifunctional reagent having an amine-reactive group and a sulfhydryl reactive group as described in, e.g., U.S. Pat. No. 4,433,059; bispecific antibody determinants generated by recombining half antibodies (heavy-light chain pairs or Fabs) from different antibodies through cycle of reduction and oxidation of disulfide bonds between the two heavy chains, as described in, e.g., U.S. Pat. No. 4,444,878; trifunctional antibodies, e.g., three Fab′ fragments cross-linked through sulfhydryl reactive groups, as described in, e.g., U.S. Pat. No. 5,273,743; biosynthetic binding proteins, e.g., pair of scFvs cross-linked through C-terminal tails preferably through disulfide or amine-reactive chemical cross-linking, as described in, e.g., U.S. Pat. No. 5,534,254; bifunctional antibodies, e.g., Fab fragments with different binding specificities dimerized through leucine zippers (e.g., c-fos and c-jun) that have replaced the constant domain, as described in, e.g., U.S. Pat. No. 5,582,996; bispecific and oligospecific mono- and oligovalent receptors, e.g., VH-CH1 regions of two antibodies (two Fab fragments) linked through a polypeptide spacer between the CH1 region of one antibody and the VH region of the other antibody typically with associated light chains, as described in, e.g., U.S. Pat. No. 5,591,828; bispecific DNA-antibody conjugates, e.g., crosslinking of antibodies or Fab fragments through a double stranded piece of DNA, as described in, e.g., U.S. Pat. No. 5,635,602; bispecific fusion proteins, e.g., an expression construct containing two scFvs with a hydrophilic helical peptide linker between them and a full constant region, as described in, e.g., U.S. Pat. No. 5,637,481; multivalent and multispecific binding proteins, e.g., dimer of polypeptides having first domain with binding region of Ig heavy chain variable region, and second domain with binding region of Ig light chain variable region, generally termed diabodies (higher order structures are also encompassed creating for bispecific, trispecific, or tetraspecific molecules, as described in, e.g., U.S. Pat. No. 5,837,242; minibody constructs with linked VL and VH chains further connected with peptide spacers to an antibody hinge region and CH3 region, which can be dimerized to form bispecific/multivalent molecules, as described in, e.g., U.S. Pat. No. 5,837,821; VH and VL domains linked with a short peptide linker (e.g., 5 or 10 amino acids) or no linker at all in either orientation, which can form dimers to form bispecific diabodies; trimers and tetramers, as described in, e.g., U.S. Pat. No. 5,844,094; String of VH domains (or VL domains in family members) connected by peptide linkages with crosslinkable groups at the C-terminus further associated with VL domains to form a series of FVs (or scFvs), as described in, e.g., U.S. Pat. No. 5,864,019; and single chain binding polypeptides with both a VH and a VL domain linked through a peptide linker are combined into multivalent structures through non-covalent or chemical crosslinking to form, e.g., homobivalent, heterobivalent, trivalent, and tetravalent structures using both scFV or diabody type format, as described in, e.g., U.S. Pat. No. 5,869,620. Additional exemplary multispecific and bispecific molecules and methods of making the same are found, for example, in U.S. Pat. Nos. 5,910,573, 5,932,448, 5,959,083, 5,989,830, 6,005,079, 6,239,259, 6,294,353, 6,333,396, 6,476,198, 6,511,663, 6,670,453, 6,743,896, 6,809,185, 6,833,441, 7,129,330, 7,183,076, 7,521,056, 7,527,787, 7,534,866, 7,612,181, US2002004587A1, US2002076406A1, US2002103345A1, US2003207346A1, US2003211078A1, US2004219643A1, US2004220388A1, US2004242847A1, US2005003403A1, US2005004352A1, US2005069552A1, US2005079170A1, US2005100543A1, US2005136049A1, US2005136051A1, US2005163782A1, US2005266425A1, US2006083747A1, US2006120960A1, US2006204493A1, US2006263367A1, US2007004909A1, US2007087381A1, US2007128150A1, US2007141049A1, US2007154901A1, US2007274985A1, US2008050370A1, US2008069820A1, US2008152645A1, US2008171855A1, US2008241884A1, US2008254512A1, US2008260738A1, US2009130106A1, US2009148905A1, US2009155275A1, US2009162359A1, US2009162360A1, US2009175851A1, US2009175867A1, US2009232811A1, US2009234105A1, US2009263392A1, US2009274649A1, EP346087A2, WO0006605A2, WO02072635A2, WO04081051A1, WO06020258A2, WO2007044887A2, WO2007095338A2, WO2007137760A2, WO2008119353A1, WO2009021754A2, WO2009068630A1, WO9103493A1, WO9323537A1, WO9409131A1, WO9412625A2, WO9509917A1, WO9637621A2, WO9964460A1. The contents of the above-referenced applications are incorporated herein by reference in their entireties.

Within each antibody or antibody fragment (e.g., scFv) of a bispecific antibody molecule, the VH can be upstream or downstream of the VL. In some embodiments, the upstream antibody or antibody fragment (e.g., scFv) is arranged with its VH (VH₁) upstream of its VL (VL₁) and the downstream antibody or antibody fragment (e.g., scFv) is arranged with its VL (VL₂) upstream of its VH (VH₂), such that the overall bispecific antibody molecule has the arrangement VH₁-VL₁-VL₂-VH₂. In other embodiments, the upstream antibody or antibody fragment (e.g., scFv) is arranged with its VL (VL₁) upstream of its VH (VH₁) and the downstream antibody or antibody fragment (e.g., scFv) is arranged with its VH (VH₂) upstream of its VL (VL₂), such that the overall bispecific antibody molecule has the arrangement VL₁-VH₁-VH₂-VL₂. Optionally, a linker is disposed between the two antibodies or antibody fragments (e.g., scFvs), e.g., between VL₁ and VL₂ if the construct is arranged as VH₁-VL₁-VL₂-VH₂, or between VH₁ and VH₂ if the construct is arranged as VL₁-VH₁-VH₂-VL₂. The linker may be a linker as described herein, e.g., a (Gly4-Ser)n linker, wherein n is 1, 2, 3, 4, 5, or 6, preferably 4 (SEQ ID NO: 72). In general, the linker between the two scFvs should be long enough to avoid mispairing between the domains of the two scFvs. Optionally, a linker is disposed between the VL and VH of the first scFv. Optionally, a linker is disposed between the VL and VH of the second scFv. In constructs that have multiple linkers, any two or more of the linkers can be the same or different. Accordingly, in some embodiments, a bispecific CAR comprises VLs, VHs, and optionally one or more linkers in an arrangement as described herein.

Stability and Mutations

The stability of an antigen binding domain to a cancer associated antigen as described herein, e.g., scFv molecules (e.g., soluble scFv), can be evaluated in reference to the biophysical properties (e.g., thermal stability) of a conventional control scFv molecule or a full length antibody. In one embodiment, the humanized scFv has a thermal stability that is greater than about 0.1, about 0.25, about 0.5, about 0.75, about 1, about 1.25, about 1.5, about 1.75, about 2, about 2.5, about 3, about 3.5, about 4, about 4.5, about 5, about 5.5, about 6, about 6.5, about 7, about 7.5, about 8, about 8.5, about 9, about 9.5, about 10 degrees, about 11 degrees, about 12 degrees, about 13 degrees, about 14 degrees, or about 15 degrees Celsius than a control binding molecule (e.g. a conventional scFv molecule) in the described assays.

The improved thermal stability of the antigen binding domain to a cancer associated antigen described herein, e.g., scFv is subsequently conferred to the entire CAR construct, leading to improved therapeutic properties of the CAR construct. The thermal stability of the antigen binding domain of—a cancer associated antigen described herein, e.g., scFv, can be improved by at least about 2° C. or 3° C. as compared to a conventional antibody. In one embodiment, the antigen binding domain of—a cancer associated antigen described herein, e.g., scFv, has a 1° C. improved thermal stability as compared to a conventional antibody. In another embodiment, the antigen binding domain of a cancer associated antigen described herein, e.g., scFv, has a 2° C. improved thermal stability as compared to a conventional antibody. In another embodiment, the scFv has a 4, 5, 6, 7, 8, 9, 10, 11, 12, 13, 14, 15° C. improved thermal stability as compared to a conventional antibody. Comparisons can be made, for example, between the scFv molecules disclosed herein and scFv molecules or Fab fragments of an antibody from which the scFv VH and VL were derived. Thermal stability can be measured using methods known in the art. For example, in one embodiment, Tm can be measured. Methods for measuring Tm and other methods of determining protein stability are described in more detail below.

Mutations in scFv (arising through humanization or direct mutagenesis of the soluble scFv) can alter the stability of the scFv and improve the overall stability of the scFv and the CAR construct. Stability of the humanized scFv is compared against the murine scFv using measurements such as Tm, temperature denaturation and temperature aggregation.

The binding capacity of the mutant scFvs can be determined using assays know in the art and described herein.

In one embodiment, the antigen binding domain of—a cancer associated antigen described herein, e.g., scFv, comprises at least one mutation arising from the humanization process such that the mutated scFv confers improved stability to the CAR construct. In another embodiment, the antigen binding domain of—a cancer associated antigen described herein, e.g., scFv, comprises at least 1, 2, 3, 4, 5, 6, 7, 8, 9, 10 mutations arising from the humanization process such that the mutated scFv confers improved stability to the CAR construct.

Methods of Evaluating Protein Stability

The stability of an antigen binding domain may be assessed using, e.g., the methods described below. Such methods allow for the determination of multiple thermal unfolding transitions where the least stable domain either unfolds first or limits the overall stability threshold of a multidomain unit that unfolds cooperatively (e.g., a multidomain protein which exhibits a single unfolding transition). The least stable domain can be identified in a number of additional ways. Mutagenesis can be performed to probe which domain limits the overall stability. Additionally, protease resistance of a multidomain protein can be performed under conditions where the least stable domain is known to be intrinsically unfolded via DSC or other spectroscopic methods (Fontana, et al., (1997) Fold. Des., 2: R17-26; Dimasi et al. (2009) J. Mol. Biol. 393: 672-692). Once the least stable domain is identified, the sequence encoding this domain (or a portion thereof) may be employed as a test sequence in the methods.

a) Thermal Stability

The thermal stability of the compositions may be analyzed using a number of non-limiting biophysical or biochemical techniques known in the art. In certain embodiments, thermal stability is evaluated by analytical spectroscopy.

An exemplary analytical spectroscopy method is Differential Scanning calorimetry (DSC). DSC employs a calorimeter which is sensitive to the heat absorbances that accompany the unfolding of most proteins or protein domains (see, e.g. Sanchez-Ruiz, et al., Biochemistry, 27: 1648-52, 1988). To determine the thermal stability of a protein, a sample of the protein is inserted into the calorimeter and the temperature is raised until the Fab or scFv unfolds. The temperature at which the protein unfolds is indicative of overall protein stability.

Another exemplary analytical spectroscopy method is Circular Dichroism (CD) spectroscopy. CD spectrometry measures the optical activity of a composition as a function of increasing temperature. Circular dichroism (CD) spectroscopy measures differences in the absorption of left-handed polarized light versus right-handed polarized light which arise due to structural asymmetry. A disordered or unfolded structure results in a CD spectrum very different from that of an ordered or folded structure. The CD spectrum reflects the sensitivity of the proteins to the denaturing effects of increasing temperature and is therefore indicative of a protein's thermal stability (see van Mierlo and Steemsma, J. Biotechnol., 79(3):281-98, 2000).

Another exemplary analytical spectroscopy method for measuring thermal stability is Fluorescence Emission Spectroscopy (see van Mierlo and Steemsma, supra). Yet another exemplary analytical spectroscopy method for measuring thermal stability is Nuclear Magnetic Resonance (NMR) spectroscopy (see, e.g. van Mierlo and Steemsma, supra).

The thermal stability of a composition can be measured biochemically. An exemplary biochemical method for assessing thermal stability is a thermal challenge assay. In a “thermal challenge assay”, a composition is subjected to a range of elevated temperatures for a set period of time. For example, in one embodiment, test scFv molecules or molecules comprising scFv molecules are subject to a range of increasing temperatures, e.g., for 1-1.5 hours. The activity of the protein is then assayed by a relevant biochemical assay. For example, if the protein is a binding protein (e.g. an scFv or scFv-containing polypeptide) the binding activity of the binding protein may be determined by a functional or quantitative ELISA.

Such an assay may be done in a high-throughput format and those disclosed in the Examples using E. coli and high throughput screening. A library of antigen binding domains, e.g., that includes an antigen binding domain to—a cancer associated antigen described herein, e.g., scFv variants, may be created using methods known in the art. Antigen binding domain, e.g., to—a cancer associated antigen described herein, e.g., scFv, expression may be induced and the antigen binding domain, e.g., to—a cancer associated antigen described herein, e.g., scFv, may be subjected to thermal challenge. The challenged test samples may be assayed for binding and those antigen binding domains to—a cancer associated antigen described herein, e.g., scFvs, which are stable may be scaled up and further characterized.

Thermal stability is evaluated by measuring the melting temperature (Tm) of a composition using any of the above techniques (e.g. analytical spectroscopy techniques). The melting temperature is the temperature at the midpoint of a thermal transition curve wherein 50% of molecules of a composition are in a folded state (See e.g., Dimasi et al. (2009) J. Mol Biol. 393: 672-692). In one embodiment, Tm values for an antigen binding domain to—a cancer associated antigen described herein, e.g., scFv, are about 40° C., 41° C., 42° C., 43° C., 44° C., 45° C., 46° C., 47° C., 48° C., 49° C., 50° C., 51° C., 52° C., 53° C., 54° C., 55° C., 56° C., 57° C., 58° C., 59° C., 60° C., 61° C., 62° C., 63° C., 64° C., 65° C., 66° C., 67° C., 68° C., 69° C., 70° C., 71° C., 72° C., 73° C., 74° C., 75° C., 76° C., 77° C., 78° C., 79° C., 80° C., 81° C., 82° C., 83° C., 84° C., 85° C., 86° C., 87° C., 88° C., 89° C., 90° C., 91° C., 92° C., 93° C., 94° C., 95° C., 96° C., 97° C., 98° C., 99° C., 100° C. In one embodiment, Tm values for an IgG is about 40° C., 41° C., 42° C., 43° C., 44° C., 45° C., 46° C., 47° C., 48° C., 49° C., 50° C., 51° C., 52° C., 53° C., 54° C., 55° C., 56° C., 57° C., 58° C., 59° C., 60° C., 61° C., 62° C., 63° C., 64° C., 65° C., 66° C., 67° C., 68° C., 69° C., 70° C., 71° C., 72° C., 73° C., 74° C., 75° C., 76° C., 77° C., 78° C., 79° C., 80° C., 81° C., 82° C., 83° C., 84° C., 85° C., 86° C., 87° C., 88° C., 89° C., 90° C., 91° C., 92° C., 93° C., 94° C., 95° C., 96° C., 97° C., 98° C., 99° C., 100° C. In one embodiment, Tm values for an multivalent antibody is about 40° C., 41° C., 42° C., 43° C., 44° C., 45° C., 46° C., 47° C., 48° C., 49° C., 50° C., 51° C., 52° C., 53° C., 54° C., 55° C., 56° C., 57° C., 58° C., 59° C., 60° C., 61° C., 62° C., 63° C., 64° C., 65° C., 66° C., 67° C., 68° C., 69° C., 70° C., 71° C., 72° C., 73° C., 74° C., 75° C., 76° C., 77° C., 78° C., 79° C., 80° C., 81° C., 82° C., 83° C., 84° C., 85° C., 86° C., 87° C., 88° C., 89° C., 90° C., 91° C., 92° C., 93° C., 94° C., 95° C., 96° C., 97° C., 98° C., 99° C., 100° C.

Thermal stability is also evaluated by measuring the specific heat or heat capacity (Cp) of a composition using an analytical calorimetric technique (e.g. DSC). The specific heat of a composition is the energy (e.g. in kcal/mol) is required to rise by 1° C., the temperature of 1 mol of water. As large Cp is a hallmark of a denatured or inactive protein composition. The change in heat capacity (deltaCp) of a composition is measured by determining the specific heat of a composition before and after its thermal transition. Thermal stability may also be evaluated by measuring or determining other parameters of thermodynamic stability including Gibbs free energy of unfolding (G), enthalpy of unfolding (H), or entropy of unfolding (S). One or more of the above biochemical assays (e.g. a thermal challenge assay) are used to determine the temperature (i.e. the T_{C} value) at which 50% of the composition retains its activity (e.g. binding activity).

In addition, mutations to the antigen binding domain of a cancer associated antigen described herein, e.g., scFv, can be made to alter the thermal stability of the antigen binding domain of a cancer associated antigen described herein, e.g., scFv, as compared with the unmutated antigen binding domain of a cancer associated antigen described herein, e.g., scFv. When the humanized antigen binding domain of a cancer associated antigen described herein, e.g., scFv, is incorporated into a CAR construct, the antigen binding domain of the cancer associated antigen described herein, e.g., humanized scFv, confers thermal stability to the overall CARs of the present invention. In one embodiment, the antigen binding domain to a cancer associated antigen described herein, e.g., scFv, comprises a single mutation that confers thermal stability to the antigen binding domain of the cancer associated antigen described herein, e.g., scFv. In another embodiment, the antigen binding domain to a cancer associated antigen described herein, e.g., scFv, comprises multiple mutations that confer thermal stability to the antigen binding domain to the cancer associated antigen described herein, e.g., scFv. In one embodiment, the multiple mutations in the antigen binding domain to a cancer associated antigen described herein, e.g., scFv, have an additive effect on thermal stability of the antigen binding domain to the cancer associated antigen described herein binding domain, e.g., scFv.

b) % Aggregation

The stability of a composition can be determined by measuring its propensity to aggregate. Aggregation can be measured by a number of non-limiting biochemical or biophysical techniques. For example, the aggregation of a composition may be evaluated using chromatography, e.g. Size-Exclusion Chromatography (SEC). SEC separates molecules on the basis of size. A column is filled with semi-solid beads of a polymeric gel that will admit ions and small molecules into their interior but not large ones. When a protein composition is applied to the top of the column, the compact folded proteins (i.e. non-aggregated proteins) are distributed through a larger volume of solvent than is available to the large protein aggregates. Consequently, the large aggregates move more rapidly through the column, and in this way the mixture can be separated or fractionated into its components. Each fraction can be separately quantified (e.g. by light scattering) as it elutes from the gel. Accordingly, the % aggregation of a composition can be determined by comparing the concentration of a fraction with the total concentration of protein applied to the gel. Stable compositions elute from the column as essentially a single fraction and appear as essentially a single peak in the elution profile or chromatogram.

c) Binding Affinity

The stability of a composition can be assessed by determining its target binding affinity. A wide variety of methods for determining binding affinity are known in the art. An exemplary method for determining binding affinity employs surface plasmon resonance. Surface plasmon resonance is an optical phenomenon that allows for the analysis of real-time biospecific interactions by detection of alterations in protein concentrations within a biosensor matrix, for example using the BIAcore system (Pharmacia Biosensor AB, Uppsala, Sweden and Piscataway, N.J.). For further descriptions, see Jonsson, U., et al. (1993) Ann. Biol. Clin. 51:19-26; Jonsson, U., i (1991) Biotechniques 11:620-627; Johnsson, B., et al. (1995) J. Mol. Recognit. 8:125-131; and Johnnson, B., et al. (1991) Anal. Biochem. 198:268-277.

In one aspect, the antigen binding domain of the CAR comprises an amino acid sequence that is homologous to an antigen binding domain amino acid sequence described herein, and the antigen binding domain retains the desired functional properties of the antigen binding domain described herein.

In one specific aspect, the CAR composition of the invention comprises an antibody fragment. In a further aspect, the antibody fragment comprises an scFv.

In various aspects, the antigen binding domain of the CAR is engineered by modifying one or more amino acids within one or both variable regions (e.g., VH and/or VL), for example within one or more CDR regions and/or within one or more framework regions. In one specific aspect, the CAR composition of the invention comprises an antibody fragment. In a further aspect, the antibody fragment comprises an scFv.

It will be understood by one of ordinary skill in the art that the antibody or antibody fragment of the invention may further be modified such that they vary in amino acid sequence (e.g., from wild-type), but not in desired activity. For example, additional nucleotide substitutions leading to amino acid substitutions at “non-essential” amino acid residues may be made to the protein For example, a nonessential amino acid residue in a molecule may be replaced with another amino acid residue from the same side chain family. In another embodiment, a string of amino acids can be replaced with a structurally similar string that differs in order and/or composition of side chain family members, e.g., a conservative substitution, in which an amino acid residue is replaced with an amino acid residue having a similar side chain, may be made.

Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

Percent identity in the context of two or more nucleic acids or polypeptide sequences, refers to two or more sequences that are the same. Two sequences are “substantially identical” if two sequences have a specified percentage of amino acid residues or nucleotides that are the same (e.g., 60% identity, optionally 70%, 71%. 72%. 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identity over a specified region, or, when not specified, over the entire sequence), when compared and aligned for maximum correspondence over a comparison window, or designated region as measured using one of the following sequence comparison algorithms or by manual alignment and visual inspection. Optionally, the identity exists over a region that is at least about 50 nucleotides (or 10 amino acids) in length, or more preferably over a region that is 100 to 500 or 1000 or more nucleotides (or 20, 50, 200 or more amino acids) in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. Methods of alignment of sequences for comparison are well known in the art. Optimal alignment of sequences for comparison can be conducted, e.g., by the local homology algorithm of Smith and Waterman, (1970) Adv. Appl. Math. 2:482c, by the homology alignment algorithm of Needleman and Wunsch, (1970) J. Mol. Biol. 48:443, by the search for similarity method of Pearson and Lipman, (1988) Proc. Nat'l. Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Brent et al., (2003) Current Protocols in Molecular Biology).

Two examples of algorithms that are suitable for determining percent sequence identity and sequence similarity are the BLAST and BLAST 2.0 algorithms, which are described in Altschul et al., (1977) Nuc. Acids Res. 25:3389-3402; and Altschul et al., (1990) J. Mol. Biol. 215:403-410, respectively. Software for performing BLAST analyses is publicly available through the National Center for Biotechnology Information.

The percent identity between two amino acid sequences can also be determined using the algorithm of E. Meyers and W. Miller, (1988) Comput. Appl. Biosci. 4:11-17) which has been incorporated into the ALIGN program (version 2.0), using a PAM120 weight residue table, a gap length penalty of 12 and a gap penalty of 4. In addition, the percent identity between two amino acid sequences can be determined using the Needleman and Wunsch (1970) J. Mol. Biol. 48:444-453) algorithm which has been incorporated into the GAP program in the GCG software package (available at www.gcg.com), using either a Blossom 62 matrix or a PAM250 matrix, and a gap weight of 16, 14, 12, 10, 8, 6, or 4 and a length weight of 1, 2, 3, 4, 5, or 6.

In one aspect, the present invention contemplates modifications of the starting antibody or fragment (e.g., scFv) amino acid sequence that generate functionally equivalent molecules. For example, the VH or VL of an antigen binding domain to—a cancer associated antigen described herein, e.g., scFv, comprised in the CAR can be modified to retain at least about 70%, 71%. 72%. 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identity of the starting VH or VL framework region of the antigen binding domain to the cancer associated antigen described herein, e.g., scFv. The present invention contemplates modifications of the entire CAR construct, e.g., modifications in one or more amino acid sequences of the various domains of the CAR construct in order to generate functionally equivalent molecules. The CAR construct can be modified to retain at least about 70%, 71%. 72%. 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identity of the starting CAR construct.

Transmembrane Domain

With respect to the transmembrane domain, in various embodiments, a CAR can be designed to comprise a transmembrane domain that is attached to the extracellular domain of the CAR. A transmembrane domain can include one or more additional amino acids adjacent to the transmembrane region, e.g., one or more amino acid associated with the extracellular region of the protein from which the transmembrane was derived (e.g., 1, 2, 3, 4, 5, 6, 7, 8, 9, 10 up to 15 amino acids of the extracellular region) and/or one or more additional amino acids associated with the intracellular region of the protein from which the transmembrane protein is derived (e.g., 1, 2, 3, 4, 5, 6, 7, 8, 9, 10 up to 15 amino acids of the intracellular region). In one aspect, the transmembrane domain is one that is associated with one of the other domains of the CAR, e.g., in one embodiment, the transmembrane domain may be from the same protein that the signaling domain, costimulatory domain or the hinge domain is derived from. In another aspect, the transmembrane domain is not derived from the same protein that any other domain of the CAR is derived from. In some instances, the transmembrane domain can be selected or modified by amino acid substitution to avoid binding of such domains to the transmembrane domains of the same or different surface membrane proteins, e.g., to minimize interactions with other members of the receptor complex. In one aspect, the transmembrane domain is capable of homodimerization with another CAR on the cell surface of a CAR-expressing cell. In a different aspect the amino acid sequence of the transmembrane domain may be modified or substituted so as to minimize interactions with the binding domains of the native binding partner present in the same CAR-expressing cell.

The transmembrane domain may be derived either from a natural or from a recombinant source. Where the source is natural, the domain may be derived from any membrane-bound or transmembrane protein. In one aspect the transmembrane domain is capable of signaling to the intracellular domain(s) whenever the CAR has bound to a target. A transmembrane domain of particular use in this invention may include at least the transmembrane region(s) of e.g., the alpha, beta or zeta chain of the T-cell receptor, CD28, CD3 epsilon, CD45, CD4, CD5, CD8, CD9, CD16, CD22, CD33, CD37, CD64, CD80, CD86, CD134, CD137, CD154. In some embodiments, a transmembrane domain may include at least the transmembrane region(s) of, e.g., KIRDS2, OX40, CD2, CD27, LFA-1 (CD11a, CD18), ICOS (CD278), 4-1BB (CD137), GITR, CD40, BAFFR, HVEM (LIGHTR), SLAMF7, NKp80 (KLRF1), NKp44, NKp30, NKp46, CD160, CD19, IL2R beta, IL2R gamma, IL7Rα, ITGA1, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), SLAMF6 (NTB-A, Ly108), SLAM (SLAMF1, CD150, IPO-3), BLAME (SLAMF8), SELPLG (CD162), LTBR, PAG/Cbp, NKG2D, NKG2C, or CD19.

In some instances, the transmembrane domain can be attached to the extracellular region of the CAR, e.g., the antigen binding domain of the CAR, via a hinge, e.g., a hinge from a human protein. For example, in one embodiment, the hinge can be a human Ig (immunoglobulin) hinge, e.g., an IgG4 hinge, an IgD hinge, a GS linker (e.g., a GS linker described herein), a KIR2DS2 hinge, or a CD8a hinge. In one embodiment, the hinge or spacer comprises (e.g., consists of) the amino acid sequence of SEQ ID NO:14. In one aspect, the transmembrane domain comprises (e.g., consists of) a transmembrane domain of SEQ ID NO: 15.

In one aspect, the hinge or spacer comprises an IgG4 hinge. For example, in one embodiment, the hinge or spacer comprises a hinge of the amino acid sequence ESKYGPPCPPCPAPEFLGGPSVFLFPPKPKDTLMISRTPEVTCVVVDVSQEDPEVQFNWY VDGVEVHNAKTKPREEQFNSTYRVVSVLTVLHQDWLNGKEYKCKVSNKGLPSSIEKTI SKAKGQPREPQVYTLPPSQEEMTKNQVSLTCLVKGFYPSDIAVEWESNGQPENNYKTTP PVLDSDGSFFLYSRLTVDKSRWQEGNVFSCSVMHEALHNHYTQKSLSLSLGKM (SEQ ID NO:45). In some embodiments, the hinge or spacer comprises a hinge encoded by a nucleotide sequence of

(SEQ ID NO: 46)

GAGAGCAAGTACGGCCCTCCCTGCCCCCCTTGCCCTGCCCCCGAGTTCCT
GGGCGGACCCAGCGTGTTCCTGTTCCCCCCCAAGCCCAAGGACACCCTGA
TGATCAGCCGGACCCCCGAGGTGACCTGTGTGGTGGTGGACGTGTCCCAG
GAGGACCCCGAGGTCCAGTTCAACTGGTACGTGGACGGCGTGGAGGTGCA
CAACGCCAAGACCAAGCCCCGGGAGGAGCAGTTCAATAGCACCTACCGGG
TGGTGTCCGTGCTGACCGTGCTGCACCAGGACTGGCTGAACGGCAAGGAA
TACAAGTGTAAGGTGTCCAACAAGGGCCTGCCCAGCAGCATCGAGAAAAC
CATCAGCAAGGCCAAGGGCCAGCCTCGGGAGCCCCAGGTGTACACCCTGC
CCCCTAGCCAAGAGGAGATGACCAAGAACCAGGTGTCCCTGACCTGCCTG
GTGAAGGGCTTCTACCCCAGCGACATCGCCGTGGAGTGGGAGAGCAACGG
CCAGCCCGAGAACAACTACAAGACCACCCCCCCTGTGCTGGACAGCGACG
GCAGCTTCTTCCTGTACAGCCGGCTGACCGTGGACAAGAGCCGGTGGCAG
GAGGGCAACGTCTTTAGCTGCTCCGTGATGCACGAGGCCCTGCACAACCA
CTACACCCAGAAGAGCCTGAGCCTGTCCCTGGGCAAGATG.

In one aspect, the hinge or spacer comprises an IgD hinge. For example, in one embodiment, the hinge or spacer comprises a hinge of the amino acid sequence RWPESPKAQASSVPTAQPQAEGSLAKATTAPATTRNTGRGGEEKKKEKEKEEQEERET KTPECPSHTQPLGVYLLTPAVQDLWLRDKATFTCFVVGSDLKDAHLTWEVAGKVPTG GVEEGLLERHSNGSQSQHSRLTLPRSLWNAGTSVTCTLNHPSLPPQRLMALREPAAQAP VKLSLNLLASSDPPEAASWLLCEVSGFSPPNILLMWLEDQREVNTS GFAPARPPPQPGST TFWAWSVLRVPAPPSPQPATYTCVVSHEDSRTLLNASRSLEVSYVTDH (SEQ ID NO:47). In some embodiments, the hinge or spacer comprises a hinge encoded by a nucleotide sequence of

(SEQ ID NO: 48)

AGGTGGCCCGAAAGTCCCAAGGCCCAGGCATCTAGTGTTCCTACTGCACA
GCCCCAGGCAGAAGGCAGCCTAGCCAAAGCTACTACTGCACCTGCCACTA
CGCGCAATACTGGCCGTGGCGGGGAGGAGAAGAAAAAGGAGAAAGAGAAA
GAAGAACAGGAAGAGAGGGAGACCAAGACCCCTGAATGTCCATCCCATAC
CCAGCCGCTGGGCGTCTATCTCTTGACTCCCGCAGTACAGGACTTGTGGC
TTAGAGATAAGGCCACCTTTACATGTTTCGTCGTGGGCTCTGACCTGAAG
GATGCCCATTTGACTTGGGAGGTTGCCGGAAAGGTACCCACAGGGGGGGT
TGAGGAAGGGTTGCTGGAGCGCCATTCCAATGGCTCTCAGAGCCAGCACT
CAAGACTCACCCTTCCGAGATCCCTGTGGAACGCCGGGACCTCTGTCACA
TGTACTCTAAATCATCCTAGCCTGCCCCCACAGCGTCTGATGGCCCTTAG
AGAGCCAGCCGCCCAGGCACCAGTTAAGCTTAGCCTGAATCTGCTCGCCA
GTAGTGATCCCCCAGAGGCCGCCAGCTGGCTCTTATGCGAAGTGTCCGGC
TTTAGCCCGCCCAACATCTTGCTCATGTGGCTGGAGGACCAGCGAGAAGT
GAACACCAGCGGCTTCGCTCCAGCCCGGCCCCCACCCCAGCCGGGTTCTA
CCACATTCTGGGCCTGGAGTGTCTTAAGGGTCCCAGCACCACCTAGCCCC
CAGCCAGCCACATACACCTGTGTTGTGTCCCATGAAGATAGCAGGACCCT
GCTAAATGCTTCTAGGAGTCTGGAGGTTTCCTACGTGACTGACCATT.

In one aspect, the transmembrane domain may be recombinant, in which case it will comprise predominantly hydrophobic residues such as leucine and valine. In one aspect a triplet of phenylalanine, tryptophan and valine can be found at each end of a recombinant transmembrane domain.

Optionally, a short oligo- or polypeptide linker, between 2 and 10 amino acids in length may form the linkage between the transmembrane domain and the cytoplasmic region of the CAR. A glycine-serine doublet provides a particularly suitable linker. For example, in one aspect, the linker comprises the amino acid sequence of GGGGSGGGGS (SEQ ID NO:49). In some embodiments, the linker is encoded by a nucleotide sequence of

(SEQ ID NO: 50)

GGTGGCGGAGGTTCTGGAGGTGGAGGTTCC.

In one aspect, the hinge or spacer comprises a KIR2DS2 hinge.

Cytoplasmic Domain

The cytoplasmic domain or region of the CAR includes an intracellular signaling domain. An intracellular signaling domain is generally responsible for activation of at least one of the normal effector functions of the immune cell in which the CAR has been introduced.

Examples of intracellular signaling domains for use in the CAR of the invention include the cytoplasmic sequences of the T cell receptor (TCR) and co-receptors that act in concert to initiate signal transduction following antigen receptor engagement, as well as any derivative or variant of these sequences and any recombinant sequence that has the same functional capability.

It is known that signals generated through the TCR alone are insufficient for full activation of the T cell and that a secondary and/or costimulatory signal is also required. Thus, T cell activation can be said to be mediated by two distinct classes of cytoplasmic signaling sequences: those that initiate antigen-dependent primary activation through the TCR (primary intracellular signaling domains) and those that act in an antigen-independent manner to provide a secondary or costimulatory signal (secondary cytoplasmic domain, e.g., a costimulatory domain).

A primary signaling domain regulates primary activation of the TCR complex either in a stimulatory way, or in an inhibitory way. Primary intracellular signaling domains that act in a stimulatory manner may contain signaling motifs which are known as immunoreceptor tyrosine-based activation motifs or ITAMs.

Examples of ITAM containing primary intracellular signaling domains that are of particular use in the invention include those of CD3 zeta, common FcR gamma (FCER1G), Fc gamma RIIa, FcR beta (Fc Epsilon Rib), CD3 gamma, CD3 delta, CD3 epsilon, CD79a, CD79b, CD278 (also known as “ICOS”), FcεRI, DAP10, DAP12, and CD66d. In one embodiment, a CAR of the invention comprises an intracellular signaling domain, e.g., a primary signaling domain of CD3-zeta.

In one embodiment, a primary signaling domain comprises a modified ITAM domain, e.g., a mutated ITAM domain which has altered (e.g., increased or decreased) activity as compared to the native ITAM domain. In one embodiment, a primary signaling domain comprises a modified ITAM-containing primary intracellular signaling domain, e.g., an optimized and/or truncated ITAM-containing primary intracellular signaling domain. In an embodiment, a primary signaling domain comprises one, two, three, four or more ITAM motifs.

Further examples of molecules containing a primary intracellular signaling domain that are of particular use in the invention include those of DAP10, DAP12, and CD32.

### Costimulatory Signaling Domain

The intracellular signalling domain of the CAR can comprise the CD3-zeta signaling domain by itself or it can be combined with any other desired intracellular signaling domain(s) useful in the context of a CAR of the invention. For example, the intracellular signaling domain of the CAR can comprise a CD3 zeta chain portion and a costimulatory signaling domain. The costimulatory signaling domain refers to a portion of the CAR comprising the intracellular domain of a costimulatory molecule. In one embodiment, the intracellular domain is designed to comprise the signaling domain of CD3-zeta and the signaling domain of CD28. In one aspect, the intracellular domain is designed to comprise the signaling domain of CD3-zeta and the signaling domain of ICOS.

A costimulatory molecule can be a cell surface molecule other than an antigen receptor or its ligands that is required for an efficient response of lymphocytes to an antigen. Examples of such molecules include CD27, CD28, 4-1BB (CD137), OX40, CD30, CD40, PD1, ICOS, lymphocyte function-associated antigen-1 (LFA-1), CD2, CD7, LIGHT, NKG2C, B7-H3, and a ligand that specifically binds with CD83, and the like. For example, CD27 costimulation has been demonstrated to enhance expansion, effector function, and survival of human CART cells in vitro and augments human T cell persistence and antitumor activity in vivo (Song et al. Blood. 2012; 119(3):696-706). Further examples of such costimulatory molecules include MHC class I molecule, TNF receptor proteins, Immunoglobulin-like proteins, cytokine receptors, integrins, signaling lymphocytic activation molecules (SLAM proteins), activating NK cell receptors, BTLA, a Toll ligand receptor, OX40, CD2, CD7, CD27, CD28, CD30, CD40, CDS, ICAM-1, LFA-1 (CD11a/CD18), 4-1BB (CD137), B7-H3, CDS, ICAM-1, ICOS (CD278), GITR, BAFFR, LIGHT, HVEM (LIGHTR), KIRDS2, SLAMF7, NKp80 (KLRF1), NKp44, NKp30, NKp46, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, NKG2D, NKG2C, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, and a ligand that specifically binds with CD83.

The intracellular signaling sequences within the cytoplasmic portion of the CAR of the invention may be linked to each other in a random or specified order. Optionally, a short oligo- or polypeptide linker, for example, between 2 and 10 amino acids (e.g., 2, 3, 4, 5, 6, 7, 8, 9, or 10 amino acids) in length may form the linkage between intracellular signaling sequence. In one embodiment, a glycine-serine doublet can be used as a suitable linker. In one embodiment, a single amino acid, e.g., an alanine, a glycine, can be used as a suitable linker.

In one aspect, the intracellular signaling domain is designed to comprise two or more, e.g., 2, 3, 4, 5, or more, costimulatory signaling domains. In an embodiment, the two or more, e.g., 2, 3, 4, 5, or more, costimulatory signaling domains, are separated by a linker molecule, e.g., a linker molecule described herein. In one embodiment, the intracellular signaling domain comprises two costimulatory signaling domains. In some embodiments, the linker molecule is a glycine residue. In some embodiments, the linker is an alanine residue.

In one aspect, the intracellular signaling domain is designed to comprise the signaling domain of CD3-zeta and the signaling domain of CD28. In one aspect, the intracellular signaling domain is designed to comprise the signaling domain of CD3-zeta and the signaling domain of 4-1BB. In one aspect, the signaling domain of 4-1BB is a signaling domain of SEQ ID NO: 16. In one aspect, the signaling domain of CD3-zeta is a signaling domain of SEQ ID NO: 17.

In one aspect, the intracellular signaling domain is designed to comprise the signaling domain of CD3-zeta and the signaling domain of CD27. In one aspect, the signaling domain of CD27 comprises an amino acid sequence of

(SEQ ID NO: 51)

QRRKYRSNKGESPVEPAEPCRYSCPREEEGSTIPIQEDYRKPEPACSP.

In one aspect, the signalling domain of CD27 is encoded by a nucleic acid sequence of

(SEQ ID NO: 52)

AGGAGTAAGAGGAGCAGGCTCCTGCACAGTGACTACATGAACATGACTCC
CCGCCGCCCCGGGCCCACCCGCAAGCATTACCAGCCCTATGCCCCACCAC
GCGACTTCGCAGCCTATCGCTCC.

### Natural Killer Cell Receptor (NKR) CARs

In an embodiment, a CAR molecule described herein comprises one or more components of a natural killer cell receptor (NKR), thereby forming an NKR-CAR. The NKR component can be a transmembrane domain, a hinge domain, or a cytoplasmic domain from any of the following natural killer cell receptors: killer cell immunoglobulin-like receptor (KIR), e.g., KIR2DL1, KIR2DL2/L3, KIR2DL4, KIR2DL5A, KIR2DL5B, KIR2DS1, KIR2DS2, KIR2DS3, KIR2DS4, DIR2DS5, KIR3DL1/S1, KIR3DL2, KIR3DL3, KIR2DP1, and KIR3DP1; natural cytotoxicity receptor (NCR), e.g., NKp30, NKp44, NKp46; signaling lymphocyte activation molecule (SLAM) family of immune cell receptors, e.g., CD48, CD229, 2B4, CD84, NTB-A, CRACC, BLAME, and CD2F-10; Fc receptor (FcR), e.g., CD16, and CD64; and Ly49 receptors, e.g., LY49A, LY49C. The NKR-CAR molecules described herein may interact with an adaptor molecule or intracellular signaling domain, e.g., DAP12. Exemplary configurations and sequences of CAR molecules comprising NKR components are described in International Publication No. WO2014/145252, the contents of which are hereby incorporated by reference.

### Strategies for Regulating Chimeric Antigen Receptors

In some embodiments, a regulatable CAR (RCAR) where the CAR activity can be controlled is desirable to optimize the safety and efficacy of a CAR therapy. There are many ways CAR activities can be regulated. For example, inducing apoptosis using, e.g., a caspase fused to a dimerization domain (see, e.g., Di et al., N Engl. J. Med. 2011 Nov. 3; 365(18):1673-1683), can be used as a safety switch in the CAR therapy of the instant invention. In one embodiment, the cells (e.g., T cells or NK cells) expressing a CAR of the present invention further comprise an inducible apoptosis switch, wherein a human caspase (e.g., caspase 9) or a modified version is fused to a modification of the human FKB protein that allows conditional dimerization. In the presence of a small molecule, such as a rapalog (e.g., AP 1903, AP20187), the inducible caspase (e.g., caspase 9) is activated and leads to the rapid apoptosis and death of the cells (e.g., T cells or NK cells) expressing a CAR of the present invention. Examples of a caspase-based inducible apoptosis switch (or one or more aspects of such a switch) have been described in, e.g., US2004040047; US20110286980; US20140255360; WO1997031899; WO2014151960; WO2014164348; WO2014197638; WO2014197638; all of which are incorporated by reference herein.

In another example, CAR-expressing cells can also express an inducible Caspase-9 (iCaspase-9) molecule that, upon administration of a dimerizer drug (e.g., rimiducid (also called AP1903 (Bellicum Pharmaceuticals) or AP20187 (Ariad)) leads to activation of the Caspase-9 and apoptosis of the cells. The iCaspase-9 molecule contains a chemical inducer of dimerization (CID) binding domain that mediates dimerization in the presence of a CID. This results in inducible and selective depletion of CAR-expressing cells. In some cases, the iCaspase-9 molecule is encoded by a nucleic acid molecule separate from the CAR-encoding vector(s). In some cases, the iCaspase-9 molecule is encoded by the same nucleic acid molecule as the CAR-encoding vector. The iCaspase-9 can provide a safety switch to avoid any toxicity of CAR-expressing cells. See, e.g., Song et al. Cancer Gene Ther. 2008; 15(10):667-75; Clinical Trial Id. No. NCT02107963; and Di Stasi et al. N. Engl. J. Med. 2011; 365:1673-83.

Alternative strategies for regulating the CAR therapy of the instant invention include utilizing small molecules or antibodies that deactivate or turn off CAR activity, e.g., by deleting CAR-expressing cells, e.g., by inducing antibody dependent cell-mediated cytotoxicity (ADCC). For example, CAR-expressing cells described herein may also express an antigen that is recognized by molecules capable of inducing cell death, e.g., ADCC or complement-induced cell death. For example, CAR expressing cells described herein may also express a receptor capable of being targeted by an antibody or antibody fragment. Examples of such receptors include EpCAM, VEGFR, integrins (e.g., integrins αvβ3, α4, αI3/4β3, α4β7, α5β1, αvβ3, αv), members of the TNF receptor superfamily (e.g., TRAIL-R1, TRAIL-R2), PDGF Receptor, interferon receptor, folate receptor, GPNMB, ICAM-1, HLA-DR, CEA, CA-125, MUC1, TAG-72, IL-6 receptor, 5T4, GD2, GD3, CD2, CD3, CD4, CD5, CD11, CD11a/LFA-1, CD15, CD18/ITGB2, CD19, CD20, CD22, CD23/1gE Receptor, CD25, CD28, CD30, CD33, CD38, CD40, CD41, CD44, CD51, CD52, CD62L, CD74, CD80, CD125, CD147/basigin, CD152/CTLA-4, CD154/CD40L, CD195/CCR5, CD319/SLAMF7, and EGFR, and truncated versions thereof (e.g., versions preserving one or more extracellular epitopes but lacking one or more regions within the cytoplasmic domain).

For example, a CAR-expressing cell described herein may also express a truncated epidermal growth factor receptor (EGFR) which lacks signaling capacity but retains the epitope that is recognized by molecules capable of inducing ADCC, e.g., cetuximab (ERBITUX®), such that administration of cetuximab induces ADCC and subsequent depletion of the CAR-expressing cells (see, e.g., WO2011/056894, and Jonnalagadda et al., Gene Ther. 2013; 20(8)853-860). Another strategy includes expressing a highly compact marker/suicide gene that combines target epitopes from both CD32 and CD20 antigens in the CAR-expressing cells described herein, which binds rituximab, resulting in selective depletion of the CAR-expressing cells, e.g., by ADCC (see, e.g., Philip et al., Blood. 2014; 124(8)1277-1287). Other methods for depleting CAR-expressing cells described herein include administration of CAMPATH, a monoclonal anti-CD52 antibody that selectively binds and targets mature lymphocytes, e.g., CAR-expressing cells, for destruction, e.g., by inducing ADCC. In other embodiments, the CAR-expressing cell can be selectively targeted using a CAR ligand, e.g., an anti-idiotypic antibody. In some embodiments, the anti-idiotypic antibody can cause effector cell activity, e.g., ADCC or ADC activities, thereby reducing the number of CAR-expressing cells. In other embodiments, the CAR ligand, e.g., the anti-idiotypic antibody, can be coupled to an agent that induces cell killing, e.g., a toxin, thereby reducing the number of CAR-expressing cells. Alternatively, the CAR molecules themselves can be configured such that the activity can be regulated, e.g., turned on and off, as described below.

In other embodiments, a CAR-expressing cell described herein may also express a target protein recognized by the T cell depleting agent. In one embodiment, the target protein is CD20 and the T cell depleting agent is an anti-CD20 antibody, e.g., rituximab. In such embodiment, the T cell depleting agent is administered once it is desirable to reduce or eliminate the CAR-expressing cell, e.g., to mitigate the CAR induced toxicity. In other embodiments, the T cell depleting agent is an anti-CD52 antibody, e.g., alemtuzumab.

In an aspect, a RCAR comprises a set of polypeptides, typically two in the simplest embodiments, in which the components of a standard CAR described herein, e.g., an antigen binding domain and an intracellular signaling domain, are partitioned on separate polypeptides or members. In some embodiments, the set of polypeptides include a dimerization switch that, upon the presence of a dimerization molecule, can couple the polypeptides to one another, e.g., can couple an antigen binding domain to an intracellular signaling domain. In one embodiment, a CAR of the present invention utilizes a dimerization switch as those described in, e.g., WO2014127261, which is incorporated by reference herein. Additional description and exemplary configurations of such regulatable CARs are provided herein and in International Publication No. WO 2015/090229, hereby incorporated by reference in its entirety.

In some embodiments, an RCAR involves a switch domain, e.g., a FKBP switch domain, as set out SEQ ID NO: 122, or comprise a fragment of FKBP having the ability to bind with FRB, e.g., as set out in SEQ ID NO: 123. In some embodiments, the RCAR involves a switch domain comprising a FRB sequence, e.g., as set out in SEQ ID NO: 124, or a mutant FRB sequence, e.g., as set out in any of SEQ ID Nos. 125-130.

(SEQ ID NO: 122)

D V P D Y A S L G G P S S P K K K R K V S R G V Q
V E T I S P G D G R T F P K R G Q T C V V H Y T G
M L E D G K K F D S S R D R N K P F K F M L G K Q
E V I R G W E E G V A Q M S V G Q R A K L T I S P
D Y A Y G A T G H P G I I P P H A T L V F D V E L
L K L E T S Y

(SEQ ID NO: 123)

V Q V E T I S P G D G R T F P K R G Q T C V V H Y
T G M L E D G K K F D S S R D R N K P F K F M L G
K Q E V I R G W E E G V A Q M S V G Q R A K L T I
S P D Y A Y G A T G H P G I I P P H A T L V F D V
E L L K L E T S

(SEQ ID NO: 124)

ILWHEMWHEG LEEASRLYFG ERNVKGMFEV LEPLHAMMER
GPQTLKETSF NQAYGRDLME AQEWCRKYMK SGNVKDLTQA
WDLYYHVFRR ISK

TABLE 1
Exemplary mutant FRB having increased affinity
for a dimerization molecule.

| | | SEQ |
|---|---|---|
| | | ID |
| FRB mutant | Amino Acid Sequence | NO: |
| E2032I mutant | ILWHEMWHEGLIEASRLYFGERNVKGMFEVLEPLHAMMERGP | 125 |
| | QTLKETSFNQAYGRDLMEAQEWCRKYMKSGNVKDLTQAWD | |
| | LYYHVFRRISKTS | |
| E2032L mutant | ILWHEMWHEGLLEASRLYFGERNVKGMFEVLEPLHAMMERG | 126 |
| | PQTLKETSFNQAYGRDLMEAQEWCRKYMKSGNVKDLTQAW | |
| | DLYYHVFRRISKTS | |
| T2098L mutant | ILWHEMWHEGLEEASRLYFGERNVKGMFEVLEPLHAMMERG | 127 |
| | PQTLKETSFNQAYGRDLMEAQEWCRKYMKSGNVKDLLQAW | |
| | DLYYHVFRRISKTS | |
| E2032, T2098 | ILWHEMWHEGLXEASRLYFGERNVKGMFEVLEPLHAMMERG | 128 |
| mutant | PQTLKETSFNQAYGRDLMEAQEWCRKYMKSGNVKDLXQAW | |
| | DLYYHVFRRISKTS | |
| E2032I, T2098L | ILWHEMWHEGLIEASRLYFGERNVKGMFEVLEPLHAMMERGP | 129 |
| mutant | QTLKETSFNQAYGRDLMEAQEWCRKYMKSGNVKDLLQAWD | |
| | LYYHVFRRISKTS | |
| E2032L, | ILWHEMWHEGLLEASRLYFGERNVKGMFEVLEPLHAMMERG | 130 |
| T2098L | PQTLKETSFNQAYGRDLMEAQEWCRKYMKSGNVKDLLQAW | |
| mutant | DLYYHVFRRISKTS | |

### Split CAR

In some embodiments, the CAR-expressing cell uses a split CAR. The split CAR approach is described in more detail in publications WO2014/055442 and WO2014/055657. Briefly, a split CAR system comprises a cell expressing a first CAR having a first antigen binding domain and a costimulatory domain (e.g., 41BB), and the cell also expresses a second CAR having a second antigen binding domain and an intracellular signaling domain (e.g., CD3 zeta). When the cell encounters the first antigen, the costimulatory domain is activated, and the cell proliferates. When the cell encounters the second antigen, the intracellular signaling domain is activated and cell-killing activity begins. Thus, the CAR-expressing cell is only fully activated in the presence of both antigens.

### RNA Transfection

Disclosed herein are methods for producing an in vitro transcribed RNA CAR. The present invention also includes (among other things) a CAR encoding RNA construct that can be directly transfected into a cell. A method for generating mRNA for use in transfection can involve in vitro transcription (IVT) of a template with specially designed primers, followed by polyA addition, to produce a construct containing 3′ and 5′ untranslated sequence (“UTR”), a 5′ cap and/or Internal Ribosome Entry Site (IRES), the nucleic acid to be expressed, and a polyA tail, typically 50-2000 bases in length (SEQ ID NO:118).

In one aspect the CAR is encoded by a messenger RNA (mRNA). In one aspect the mRNA encoding the CAR is introduced into an immune effector cell, e.g., a T cell or a NK cell, for production of a CAR-expressing cell, e.g., a CART cell or a CAR NK cell. In one embodiment, the in vitro transcribed RNA CAR can be introduced to a cell as a form of transient transfection.

Additional method of RNA transfection are described on pages 192-196 of International Application WO 2016/164731, filed Apr. 8, 2016, which is incorporated by reference in its entirety.

### Non-Viral Delivery Methods

In some aspects, non-viral methods can be used to deliver a nucleic acid encoding a CAR described herein into a cell or tissue or a subject. In some embodiments, the non-viral method includes the use of a transposon (also called a transposable element). In some embodiments, a transposon is a piece of DNA that can insert itself at a location in a genome, for example, a piece of DNA that is capable of self-replicating and inserting its copy into a genome, or a piece of DNA that can be spliced out of a longer nucleic acid and inserted into another place in a genome.

Additional and exemplary transposons and non-viral delivery methods are described on pages 196-198 of International Application WO 2016/164731, filed Apr. 8, 2016, which is incorporated by reference in its entirety.

### Nucleic Acid Constructs Encoding a CAR

The present invention also provides nucleic acid molecules encoding one or more CAR constructs described herein, e.g., CD19 CAR, CD20 CAR, or CD22 CAR. In one aspect, the nucleic acid molecule is provided as a messenger RNA transcript. In one aspect, the nucleic acid molecule is provided as a DNA construct.

Accordingly, in one aspect, the invention pertains to an isolated nucleic acid molecule encoding a chimeric antigen receptor (CAR), wherein the CAR comprises a binding domain (e.g., that binds CD19, CD20, or CD22) a transmembrane domain, and an intracellular signaling domain comprising a stimulatory domain, e.g., a costimulatory signaling domain and/or a primary signaling domain, e.g., zeta chain.

In one embodiment, the binding domain is an anti-CD19 binding domain described herein, e.g., an anti-CD19 binding domain which comprises a sequence selected from a group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12 and SEQ ID NO:59, or a sequence with 95-99% identity thereof.

In one embodiment, the nucleic acid comprises CD22-encoding a nucleic acid set out in Table 6A on pages 364-403 of International Application WO 2016/164731, filed Apr. 8, 2016, which is incorporated by reference in its entirety, or a sequence with 95-99% identity thereof.

In one embodiment, the nucleic acid comprises CD20-encoding a nucleic acid set out in Table 11A on pages 422-446 of International Application WO 2016/164731, filed Apr. 8, 2016, which is incorporated by reference in its entirety, or a sequence with 95-99% identity thereof.

In one embodiment, the transmembrane domain is transmembrane domain of a protein selected from the group consisting of the alpha, beta or zeta chain of the T-cell receptor, CD28, CD3 epsilon, CD45, CD4, CD5, CD8, CD9, CD16, CD22, CD33, CD37, CD64, CD80, CD86, CD134, CD137 and CD154. In one embodiment, the transmembrane domain comprises a sequence of SEQ ID NO: 15, or a sequence with 95-99% identity thereof. In one embodiment, the anti-CD19 binding domain is connected to the transmembrane domain by a hinge region, e.g., a hinge described herein. In one embodiment, the hinge region comprises SEQ ID NO:14 or SEQ ID NO:45 or SEQ ID NO:47 or SEQ ID NO:49, or a sequence with 95-99% identity thereof. In one embodiment, the isolated nucleic acid molecule further comprises a sequence encoding a costimulatory domain. In one embodiment, the costimulatory domain is a functional signaling domain of a protein selected from the group consisting of OX40, CD27, CD28, CDS, ICAM-1, LFA-1 (CD11a/CD18), ICOS (CD278), and 4-1BB (CD137). In one embodiment, the costimulatory domain is a functional signaling domain of a protein selected from the group consisting of MHC class I molecule, TNF receptor proteins, Immunoglobulin-like proteins, cytokine receptors, integrins, signaling lymphocytic activation molecules (SLAM proteins), activating NK cell receptors, BTLA, a Toll ligand receptor, OX40, CD2, CD7, CD27, CD28, CD30, CD40, CDS, ICAM-1, LFA-1 (CD11a/CD18), 4-1BB (CD137), B7-H3, CDS, ICAM-1, ICOS (CD278), GITR, BAFFR, LIGHT, HVEM (LIGHTR), KIRDS2, SLAMF7, NKp80 (KLRF1), NKp44, NKp30, NKp46, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, NKG2D, NKG2C, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, and a ligand that specifically binds with CD83. In one embodiment, the costimulatory domain comprises a sequence of SEQ ID NO:16, or a sequence with 95-99% identity thereof. In one embodiment, the intracellular signaling domain comprises a functional signaling domain of 4-1BB and a functional signaling domain of CD3 zeta. In one embodiment, the intracellular signaling domain comprises the sequence of SEQ ID NO: 16 or SEQ ID NO:51, or a sequence with 95-99% identity thereof, and the sequence of SEQ ID NO: 17 or SEQ ID NO:43, or a sequence with 95-99% identity thereof, wherein the sequences comprising the intracellular signaling domain are expressed in the same frame and as a single polypeptide chain.

In another aspect, the invention pertains to an isolated nucleic acid molecule encoding a CAR construct comprising a leader sequence of SEQ ID NO: 13, a scFv domain having a sequence selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, and SEQ ID NO:59, (or a sequence with 95-99% identity thereof), a hinge region of SEQ ID NO:14 or SEQ ID NO:45 or SEQ ID NO:47 or SEQ ID NO:49 (or a sequence with 95-99% identity thereof), a transmembrane domain having a sequence of SEQ ID NO: 15 (or a sequence with 95-99% identity thereof), a 4-1BB costimulatory domain having a sequence of SEQ ID NO:16 or a CD27 costimulatory domain having a sequence of SEQ ID NO:51 (or a sequence with 95-99% identity thereof), and a CD3 zeta stimulatory domain having a sequence of SEQ ID NO:17 or SEQ ID NO:43 (or a sequence with 95-99% identity thereof).

In another aspect, the invention pertains to an isolated polypeptide molecule encoded by the nucleic acid molecule. In one embodiment, the isolated polypeptide molecule comprises a sequence selected from the group consisting of SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:59 or a sequence with 95-99% identity thereof.

In another aspect, the invention pertains to a nucleic acid molecule encoding a chimeric antigen receptor (CAR) molecule that comprises an anti-CD19 binding domain, a transmembrane domain, and an intracellular signaling domain comprising a stimulatory domain, and wherein said anti-CD19 binding domain comprises a sequence selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12 and SEQ ID NO:59, or a sequence with 95-99% identity thereof.

In one embodiment, the encoded CAR molecule (e.g., CD19 CAR, CD20 CAR, or CD22 CAR) further comprises a sequence encoding a costimulatory domain. In one embodiment, the costimulatory domain is a functional signaling domain of a protein selected from the group consisting of OX40, CD27, CD28, CDS, ICAM-1, LFA-1 (CD11a/CD18) and 4-1BB (CD137). In one embodiment, the costimulatory domain comprises a sequence of SEQ ID NO:16. In one embodiment, the transmembrane domain is a transmembrane domain of a protein selected from the group consisting of the alpha, beta or zeta chain of the T-cell receptor, CD28, CD3 epsilon, CD45, CD4, CD5, CD8, CD9, CD16, CD22, CD33, CD37, CD64, CD80, CD86, CD134, CD137 and CD154. In one embodiment, the transmembrane domain comprises a sequence of SEQ ID NO:15. In one embodiment, the intracellular signaling domain comprises a functional signaling domain of 4-1BB and a functional signaling domain of zeta. In one embodiment, the intracellular signaling domain comprises the sequence of SEQ ID NO: 16 and the sequence of SEQ ID NO: 17, wherein the sequences comprising the intracellular signaling domain are expressed in the same frame and as a single polypeptide chain. In one embodiment, the anti-CD19 binding domain is connected to the transmembrane domain by a hinge region. In one embodiment, the hinge region comprises SEQ ID NO:14. In one embodiment, the hinge region comprises SEQ ID NO:45 or SEQ ID NO:47 or SEQ ID NO:49.

In another aspect, the invention pertains to an encoded CAR molecule comprising a leader sequence of SEQ ID NO: 13, a scFv domain having a sequence selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, and SEQ ID NO:59, or a sequence with 95-99% identity thereof, a hinge region of SEQ ID NO:14 or SEQ ID NO:45 or SEQ ID NO:47 or SEQ ID NO:49, a transmembrane domain having a sequence of SEQ ID NO: 15, a 4-1BB costimulatory domain having a sequence of SEQ ID NO:16 or a CD27 costimulatory domain having a sequence of SEQ ID NO:51, and a CD3 zeta stimulatory domain having a sequence of SEQ ID NO:17 or SEQ ID NO:43. In one embodiment, the encoded CAR molecule comprises a sequence selected from a group consisting of SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, and SEQ ID NO:59, or a sequence with 95-99% identity thereof.

The nucleic acid sequences coding for the desired molecules can be obtained using recombinant methods known in the art, such as, for example by screening libraries from cells expressing the gene, by deriving the gene from a vector known to include the same, or by isolating directly from cells and tissues containing the same, using standard techniques. Alternatively, the gene of interest can be produced synthetically, rather than cloned.

The present invention also provides vectors in which a DNA of the present invention is inserted. Vectors derived from retroviruses such as the lentivirus are suitable tools to achieve long-term gene transfer since they allow long-term, stable integration of a transgene and its propagation in daughter cells. Lentiviral vectors have the added advantage over vectors derived from onco-retroviruses such as murine leukemia viruses in that they can transduce non-proliferating cells, such as hepatocytes. They also have the added advantage of low immunogenicity. A retroviral vector may also be, e.g., a gammaretroviral vector. A gammaretroviral vector may include, e.g., a promoter, a packaging signal (w), a primer binding site (PBS), one or more (e.g., two) long terminal repeats (LTR), and a transgene of interest, e.g., a gene encoding a CAR. A gammaretroviral vector may lack viral structural gens such as gag, pol, and env. Exemplary gammaretroviral vectors include Murine Leukemia Virus (MLV), Spleen-Focus Forming Virus (SFFV), and Myeloproliferative Sarcoma Virus (MPSV), and vectors derived therefrom. Other gammaretroviral vectors are described, e.g., in Tobias Maetzig et al., “Gammaretroviral Vectors: Biology, Technology and Application” Viruses. 2011 June; 3(6): 677-713.

In another embodiment, the vector comprising the nucleic acid encoding the desired CAR of the invention is an adenoviral vector (A5/35). In another embodiment, the expression of nucleic acids encoding CARs can be accomplished using of transposons such as sleeping beauty, crispr, CAS9, and zinc finger nucleases. See below June et al. 2009 Nature Reviews Immunology 9.10: 704-716, is incorporated herein by reference.

A vector may also include, e.g., a signal sequence to facilitate secretion, a polyadenylation signal and transcription terminator (e.g., from Bovine Growth Hormone (BGH) gene), an element allowing episomal replication and replication in prokaryotes (e.g. SV40 origin and ColE1 or others known in the art) and/or elements to allow selection (e.g., ampicillin resistance gene and/or zeocin marker).

In brief summary, the expression of natural or synthetic nucleic acids encoding CARs is typically achieved by operably linking a nucleic acid encoding the CAR polypeptide or portions thereof to a promoter, and incorporating the construct into an expression vector. The vectors can be suitable for replication and integration eukaryotes. Typical cloning vectors contain transcription and translation terminators, initiation sequences, and promoters useful for regulation of the expression of the desired nucleic acid sequence.

In some aspects, the expression constructs of the present invention may also be used for nucleic acid immunization and gene therapy, using standard gene delivery protocols. Methods for gene delivery are known in the art. See, e.g., U.S. Pat. Nos. 5,399,346, 5,580,859, 5,589,466, incorporated by reference herein in their entireties. In another embodiment, the invention provides a gene therapy vector.

The nucleic acid can be cloned into a number of types of vectors. For example, the nucleic acid can be cloned into a vector including, but not limited to a plasmid, a phagemid, a phage derivative, an animal virus, and a cosmid. Vectors of particular interest include expression vectors, replication vectors, probe generation vectors, and sequencing vectors.

Further, the expression vector may be provided to a cell in the form of a viral vector. Viral vector technology is well known in the art and is described, for example, in Sambrook et al., 2012, MOLECULAR CLONING: A LABORATORY MANUAL, volumes 1-4, Cold Spring Harbor Press, NY), and in other virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers, (e.g., WO 01/96584; WO 01/29058; and U.S. Pat. No. 6,326,193).

A number of viral based systems have been developed for gene transfer into mammalian cells. For example, retroviruses provide a convenient platform for gene delivery systems. A selected gene can be inserted into a vector and packaged in retroviral particles using techniques known in the art. The recombinant virus can then be isolated and delivered to cells of the subject either in vivo or ex vivo. A number of retroviral systems are known in the art. In some embodiments, adenovirus vectors are used. A number of adenovirus vectors are known in the art. In one embodiment, lentivirus vectors are used.

Additional promoter elements, e.g., enhancers, regulate the frequency of transcriptional initiation. Typically, these are located in the region 30-110 bp upstream of the start site, although a number of promoters have been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline. Depending on the promoter, it appears that individual elements can function either cooperatively or independently to activate transcription. Exemplary promoters include the CMV IE gene, EF-1α, ubiquitin C, or phosphoglycerokinase (PGK) promoters. In an embodiment, the promoter is a PGK promoter, e.g., a truncated PGK promoter as described herein.

An example of a promoter that is capable of expressing a CAR transgene in a mammalian T cell is the EF1a promoter. The native EF1a promoter drives expression of the alpha subunit of the elongation factor-1 complex, which is responsible for the enzymatic delivery of aminoacyl tRNAs to the ribosome. The EF1a promoter has been extensively used in mammalian expression plasmids and has been shown to be effective in driving CAR expression from transgenes cloned into a lentiviral vector. See, e.g., Milone et al., Mol. Ther. 17(8): 1453-1464 (2009). In one aspect, the EF1a promoter comprises the sequence provided as SEQ ID NO:100.

Another example of a promoter is the immediate early cytomegalovirus (CMV) promoter sequence. This promoter sequence is a strong constitutive promoter sequence capable of driving high levels of expression of any polynucleotide sequence operatively linked thereto. However, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the elongation factor-1□ promoter, the hemoglobin promoter, and the creatine kinase promoter. Further, the invention should not be limited to the use of constitutive promoters. Inducible promoters are also contemplated as part of the invention. The use of an inducible promoter provides a molecular switch capable of turning on expression of the polynucleotide sequence which it is operatively linked when such expression is desired, or turning off the expression when expression is not desired. Examples of inducible promoters include, but are not limited to a metallothionine promoter, a glucocorticoid promoter, a progesterone promoter, and a tetracycline promoter.

Another example of a promoter is the phosphoglycerate kinase (PGK) promoter. In embodiments, a truncated PGK promoter (e.g., a PGK promoter with one or more, e.g., 1, 2, 5, 10, 100, 200, 300, or 400, nucleotide deletions when compared to the wild-type PGK promoter sequence) may be desired. The nucleotide sequences of exemplary PGK promoters are provided below.

WT PGK Promoter:

(SEQ ID NO: 1323)

ACCCCTCTCTCCAGCCACTAAGCCAGTTGCTCCCTCGGCTGACGGCTGCA
CGCGAGGCCTCCGAACGTCTTACGCCTTGTGGCGCGCCCGTCCTTGTCCC
GGGTGTGATGGCGGGGTGTGGGGCGGAGGGCGTGGCGGGGAAGGGCCGGC
GACGAGAGCCGCGCGGGACGACTCGTCGGCGATAACCGGTGTCGGGTAGC
GCCAGCCGCGCGACGGTAACGAGGGACCGCGACAGGCAGACGCTCCCATG
ATCACTCTGCACGCCGAAGGCAAATAGTGCAGGCCGTGCGGCGCTTGGCG
TTCCTTGGAAGGGCTGAATCCCCGCCTCGTCCTTCGCAGCGGCCCCCCGG
GTGTTCCCATCGCCGCTTCTAGGCCCACTGCGACGCTTGCCTGCACTTCT
TACACGCTCTGGGTCCCAGCCGCGGCGACGCAAAGGGCCTTGGTGCGGGT
CTCGTCGGCGCAGGGACGCGTTTGGGTCCCGACGGAACCTTTTCCGCGTT
GGGGTTGGGGCACCATAAGCT

Exemplary truncated PGK Promoters:

PGK100:

(SEQ ID NO: 1324)

ACCCCTCTCTCCAGCCACTAAGCCAGTTGCTCCCTCGGCTGACGGCTGCA
CGCGAGGCCTCCGAACGTCTTACGCCTTGTGGCGCGCCCGTCCTTGTCCC
GGGTGTGATGGCGGGGTG
PGK200:

(SEQ ID NO: 1325)

ACCCCTCTCTCCAGCCACTAAGCCAGTTGCTCCCTCGGCTGACGGCTGCA
CGCGAGGCCTCCGAACGTCTTACGCCTTGTGGCGCGCCCGTCCTTGTCCC
GGGTGTGATGGCGGGGTGTGGGGCGGAGGGCGTGGCGGGGAAGGGCCGGC
GACGAGAGCCGCGCGGGACGACTCGTCGGCGATAACCGGTGTCGGGTAGC
GCCAGCCGCGCGACGGTAACG
PGK300:

(SEQ ID NO: 1326)

ACCCCTCTCTCCAGCCACTAAGCCAGTTGCTCCCTCGGCTGACGGCTGCA
CGCGAGGCCTCCGAACGTCTTACGCCTTGTGGCGCGCCCGTCCTTGTCCC
GGGTGTGATGGCGGGGTGTGGGGCGGAGGGCGTGGCGGGGAAGGGCCGGC
GACGAGAGCCGCGCGGGACGACTCGTCGGCGATAACCGGTGTCGGGTAGC
GCCAGCCGCGCGACGGTAACGAGGGACCGCGACAGGCAGACGCTCCCATG
ATCACTCTGCACGCCGAAGGCAAATAGTGCAGGCCGTGCGGCGCTTGGCG
TTCCTTGGAAGGGCTGAATCCCCG
PGK400:

(SEQ ID NO: 1327)

ACCCCTCTCTCCAGCCACTAAGCCAGTTGCTCCCTCGGCTGACGGCTGCA
CGCGAGGCCTCCGAACGTCTTACGCCTTGTGGCGCGCCCGTCCTTGTCCC
GGGTGTGATGGCGGGGTGTGGGGCGGAGGGCGTGGCGGGGAAGGGCCGGC
GACGAGAGCCGCGCGGGACGACTCGTCGGCGATAACCGGTGTCGGGTAGC
GCCAGCCGCGCGACGGTAACGAGGGACCGCGACAGGCAGACGCTCCCATG
ATCACTCTGCACGCCGAAGGCAAATAGTGCAGGCCGTGCGGCGCTTGGCG
TTCCTTGGAAGGGCTGAATCCCCGCCTCGTCCTTCGCAGCGGCCCCCCGG
GTGTTCCCATCGCCGCTTCTAGGCCCACTGCGACGCTTGCCTGCACTTCT
TACACGCTCTGGGTCCCAGCCG

A vector may also include, e.g., a signal sequence to facilitate secretion, a polyadenylation signal and transcription terminator (e.g., from Bovine Growth Hormone (BGH) gene), an element allowing episomal replication and replication in prokaryotes (e.g. SV40 origin and ColE1 or others known in the art) and/or elements to allow selection (e.g., ampicillin resistance gene and/or zeocin marker).

In order to assess the expression of a CAR polypeptide or portions thereof, the expression vector to be introduced into a cell can also contain either a selectable marker gene or a reporter gene or both to facilitate identification and selection of expressing cells from the population of cells sought to be transfected or infected through viral vectors. In other aspects, the selectable marker may be carried on a separate piece of DNA and used in a co-transfection procedure. Both selectable markers and reporter genes may be flanked with appropriate regulatory sequences to enable expression in the host cells. Useful selectable markers include, for example, antibiotic-resistance genes, such as neo and the like.

Reporter genes are used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assayed at a suitable time after the DNA has been introduced into the recipient cells. Suitable reporter genes may include genes encoding luciferase, beta-galactosidase, chloramphenicol acetyl transferase, secreted alkaline phosphatase, or the green fluorescent protein gene (e.g., Ui-Tei et al., 2000 FEBS Letters 479: 79-82). Suitable expression systems are well known and may be prepared using known techniques or obtained commercially. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

In embodiments, the vector may comprise two or more nucleic acid sequences encoding a CAR, e.g., a first CAR that binds to CD19 and a second CAR, e.g., an inhibitory CAR or a CAR that specifically binds to a second antigen, e.g., CD10, CD20, CD22, CD34, CD123, FLT-3, ROR1, CD79b, CD179b, or CD79a. In such embodiments, the two or more nucleic acid sequences encoding the CAR are encoded by a single nucleic molecule in the same frame and as a single polypeptide chain. In this aspect, the two or more CARs, can, e.g., be separated by one or more peptide cleavage sites. (e.g., an auto-cleavage site or a substrate for an intracellular protease). Examples of peptide cleavage sites include the following, wherein the GSG residues are optional:

T2A:

(SEQ ID NO: 1328)

(GSG)EGRGSLLTCGDVEENPGP
P2A:

(SEQ ID NO: 1329)

(GSG)ATNFSLLKQAGDVEENPGP
E2A:

(SEQ ID NO: 1330)

(GSG)QCTNYALLKLAGDVESNPGP
F2A:

(SEQ ID NO: 1331)

(GSG)VKQTLNFDLLKLAGDVESNPGP

Methods of introducing and expressing genes into a cell are known in the art. In the context of an expression vector, the vector can be readily introduced into a host cell, e.g., mammalian, bacterial, yeast, or insect cell by any method in the art. For example, the expression vector can be transferred into a host cell by physical, chemical, or biological means.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art. See, for example, Sambrook et al., 2012, MOLECULAR CLONING: A LABORATORY MANUAL, volumes 1-4, Cold Spring Harbor Press, NY). A suitable method for the introduction of a polynucleotide into a host cell is calcium phosphate transfection.

Biological methods for introducing a polynucleotide of interest into a host cell include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like. See, for example, U.S. Pat. Nos. 5,350,674 and 5,585,362.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle). Other methods of state-of-the-art targeted delivery of nucleic acids are available, such as delivery of polynucleotides with targeted nanoparticles or other suitable sub-micron sized delivery system.

In the case where a non-viral delivery system is utilized, an exemplary delivery vehicle is a liposome. The use of lipid formulations is contemplated for the introduction of the nucleic acids into a host cell (in vitro, ex vivo or in vivo). In another aspect, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

Lipids suitable for use can be obtained from commercial sources. For example, dimyristyl phosphatidylcholine (“DMPC”) can be obtained from Sigma, St. Louis, Mo.; dicetyl phosphate (“DCP”) can be obtained from K & K Laboratories (Plainview, N.Y.); cholesterol (“Choi”) can be obtained from Calbiochem-Behring; dimyristyl phosphatidylglycerol (“DMPG”) and other lipids may be obtained from Avanti Polar Lipids, Inc. (Birmingham, Ala.). Stock solutions of lipids in chloroform or chloroform/methanol can be stored at about −20° C. Chloroform is used as the only solvent since it is more readily evaporated than methanol. “Liposome” is a generic term encompassing a variety of single and multilamellar lipid vehicles formed by the generation of enclosed lipid bilayers or aggregates. Liposomes can be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh et al., 1991 Glycobiology 5: 505-10). However, compositions that have different structures in solution than the normal vesicular structure are also encompassed. For example, the lipids may assume a micellar structure or merely exist as nonuniform aggregates of lipid molecules. Also contemplated are lipofectamine-nucleic acid complexes.

Regardless of the method used to introduce exogenous nucleic acids into a host cell or otherwise expose a cell to the inhibitor of the present invention, in order to confirm the presence of the recombinant DNA sequence in the host cell, a variety of assays may be performed. Such assays include, for example, “molecular biological” assays well known to those of skill in the art, such as Southern and Northern blotting, RT-PCR and PCR; “biochemical” assays, such as detecting the presence or absence of a particular peptide, e.g., by immunological means (ELISAs and Western blots) or by assays described herein to identify agents falling within the scope of the invention.

The present invention further provides a vector comprising a CAR encoding nucleic acid molecule. In one aspect, a CAR vector can be directly transduced into a cell, e.g., a T cell. In one aspect, the vector is a cloning or expression vector, e.g., a vector including, but not limited to, one or more plasmids (e.g., expression plasmids, cloning vectors, minicircles, minivectors, double minute chromosomes), retroviral and lentiviral vector constructs. In one aspect, the vector is capable of expressing the CAR construct in mammalian T cells. In one aspect, the mammalian T cell is a human T cell.

### Immune Effector Cells Expressing a CAR

In another aspect, the present invention provides a population of CAR-expressing cells. In some embodiments, the population of CAR-expressing cells comprises a cell that expresses one or more CARs described herein. In some embodiments, the population of CAR-expressing cells comprises a mixture of cells expressing different CARs.

For example, in one embodiment, the population of CART cells can include a first cell expressing a CAR having an antigen binding domain to a tumor antigen described herein, e.g., CD19, and a second cell expressing a CAR having a different antigen binding domain, e.g., an antigen binding domain to a different tumor antigen described herein, e.g., an antigen binding domain to a tumor antigen described herein that differs from the tumor antigen bound by the antigen binding domain of the CAR expressed by the first cell, e.g., CD10, CD20, CD22, CD34, CD123, FLT-3, ROR1, CD79b, CD179b, or CD79a.

As another example, the population of CAR-expressing cells can include a first cell expressing a CAR that includes an antigen binding domain to a tumor antigen described herein, and a second cell expressing a CAR that includes an antigen binding domain to a target other than a tumor antigen as described herein. In one embodiment, the population of CAR-expressing cells includes, e.g., a first cell expressing a CAR that includes a primary intracellular signaling domain, and a second cell expressing a CAR that includes a secondary signaling domain. Either one or both of the CAR expressing cells can have a truncated PGK promoter, e.g., as described herein, operably linked to the nucleic acid encoding the CAR.

In another aspect, the present invention provides a population of cells wherein at least one cell in the population expresses a CAR having an antigen binding domain to a tumor antigen described herein, and a second cell expressing another agent, e.g., an agent which enhances the activity of a CAR-expressing cell. The CAR expressing cells of the population can have a truncated PGK promoter, e.g., as described herein, operably linked to the nucleic acid encoding the CAR. In one embodiment, the agent can be an agent which inhibits an inhibitory molecule. Inhibitory molecules, e.g., PD-1, can, in some embodiments, decrease the ability of a CAR-expressing cell to mount an immune effector response. Examples of inhibitory molecules include PD-1, PD-L1, PD-L2, CTLA4, TIM3, CEACAM (CEACAM-1, CEACAM-3, and/or CEACAM-5), LAG3, VISTA, BTLA, TIGIT, LAIR1, CD160, 2B4, CD80, CD86, B7-H3 (CD276), B7-H4 (VTCN1), HVEM (TNFRSF14 or CD270), KIR, A2aR, MHC class I, MHC class II, GALS, adenosine, and TGF (e.g., TGF beta). In one embodiment, the agent which inhibits an inhibitory molecule comprises a first polypeptide, e.g., an inhibitory molecule, associated with a second polypeptide that provides a positive signal to the cell, e.g., an intracellular signaling domain described herein. In one embodiment, the agent comprises a first polypeptide, e.g., of an inhibitory molecule such as PD1, PD-L1, CTLA4, TIM3, CEACAM (e.g., CEACAM-1, CEACAM-3, and/or CEACAM-5), LAG3, VISTA, BTLA, TIGIT, LAIR1, CD160, 2B4 or TGF beta, or a fragment of any of these, and a second polypeptide which is an intracellular signaling domain described herein (e.g., comprising a costimulatory domain (e.g., 41BB, CD27, OX40 or CD28, e.g., as described herein) and/or a primary signaling domain (e.g., a CD3 zeta signaling domain described herein). In one embodiment, the agent comprises a first polypeptide of PD-1 or a fragment thereof, and a second polypeptide of an intracellular signaling domain described herein (e.g., a CD28 signaling domain described herein and/or a CD3 zeta signaling domain described herein).

Co-Expression of CAR with Other Molecules or Agents

Co-Expression of a Second CAR

In one aspect, the CAR-expressing cell described herein can further comprise a second CAR, e.g., a second CAR that includes a different antigen binding domain, e.g., to the same target (CD19) or a different target (e.g., CD10, CD20, CD22, CD34, CD123, FLT-3, ROR1, CD79b, CD179b, or CD79a). In one embodiment, the second CAR includes an antigen binding domain to a target expressed on acute myeloid leukemia cells, such as, e.g., CD20, CD22, ROR1, CD10, CD33, CLL-1, CD34, CD123, FLT3, CD79b, CD179b, and CD79a. In one embodiment, the CAR-expressing cell comprises a first CAR that targets a first antigen and includes an intracellular signaling domain having a costimulatory signaling domain but not a primary signaling domain, and a second CAR that targets a second, different, antigen and includes an intracellular signaling domain having a primary signaling domain but not a costimulatory signaling domain. While not wishing to be bound by theory, placement of a costimulatory signaling domain, e.g., 4-1BB, CD28, CD27 or OX-40, onto the first CAR, and the primary signaling domain, e.g., CD3 zeta, on the second CAR can limit the CAR activity to cells where both targets are expressed. In one embodiment, the CAR expressing cell comprises a first CD19 CAR that includes a CD19 binding domain, a transmembrane domain and a costimulatory domain and a second CAR that targets an antigen other than CD19 (e.g., an antigen expressed on AML cells, e.g., CD22, CD20, ROR1, CD10, CD33, CLL-1, CD34, CD123, FLT3, CD79b, CD179b, or CD79a) and includes an antigen binding domain, a transmembrane domain and a primary signaling domain. In another embodiment, the CAR expressing cell comprises a first CD19 CAR that includes a CD19 binding domain, a transmembrane domain and a primary signaling domain and a second CAR that targets an antigen other than CD19 (e.g., an antigen expressed on AML cells, e.g., CD22, CD20, ROR1, CD10, CD33, CD123, CLL-1, CD34, FLT3, CD79b, CD179b, or CD79a) and includes an antigen binding domain to the antigen, a transmembrane domain and a costimulatory signaling domain.

In one aspect, the CAR-expressing cell described herein can further comprise a second CAR, e.g., a second CAR that includes a different antigen binding domain, e.g., to the same target (e.g., CD19) or a different target (e.g., a target other than CD19, e.g., CD10, CD20, CD22, CD34, CD123, FLT-3, ROR1, CD79b, CD179b, or CD79a). In one embodiment, the CAR-expressing cell comprises a first CAR that targets a first antigen and includes an intracellular signaling domain having a costimulatory signaling domain but not a primary signaling domain, and a second CAR that targets a second, different, antigen and includes an intracellular signaling domain having a primary signaling domain but not a costimulatory signaling domain. Placement of a costimulatory signaling domain, e.g., 4-1BB, CD28, CD27, OX-40 or ICOS, onto the first CAR, and the primary signaling domain, e.g., CD3 zeta, on the second CAR can limit the CAR activity to cells where both targets are expressed. In one embodiment, the CAR expressing cell comprises a first CAR that includes an antigen binding domain, a transmembrane domain and a costimulatory domain and a second CAR that targets another antigen and includes an antigen binding domain, a transmembrane domain and a primary signaling domain. In another embodiment, the CAR expressing cell comprises a first CAR that includes an antigen binding domain, a transmembrane domain and a primary signaling domain and a second CAR that targets another antigen and includes an antigen binding domain to the antigen, a transmembrane domain and a costimulatory signaling domain.

In one embodiment, the CAR-expressing cell comprises an XCAR described herein (e.g., CD19 CAR, CD20 CAR, or CD22 CAR) and an inhibitory CAR. In one embodiment, the CAR-expressing cell comprises a CD19 CAR described herein and an inhibitory CAR. In one embodiment, the inhibitory CAR comprises an antigen binding domain that binds an antigen found on normal cells but not cancer cells, e.g., normal cells that also express CD19. In one embodiment, the inhibitory CAR comprises the antigen binding domain, a transmembrane domain and an intracellular domain of an inhibitory molecule. For example, the intracellular domain of the inhibitory CAR can be an intracellular domain PD-1, PD-L1, PD-L2, CTLA4, TIM3, CEACAM (CEACAM-1, CEACAM-3, and/or CEACAM-5), LAGS, VISTA, BTLA, TIGIT, LAIR1, CD160, 2B4, CD80, CD86, B7-H3 (CD276), B7-H4 (VTCN1), HVEM (TNFRSF14 or CD270), KIR, A2aR, MHC class I, MHC class II, GAL9, adenosine, and TGF (e.g., TGF beta).

In one embodiment, when the CAR-expressing cell comprises two or more different CARs, the antigen binding domains of the different CARs can be such that the antigen binding domains do not interact with one another. For example, a cell expressing a first and second CAR can have an antigen binding domain of the first CAR, e.g., as a fragment, e.g., an scFv, that does not form an association with the antigen binding domain of the second CAR, e.g., the antigen binding domain of the second CAR is a VHH.

Co-Expression of an Agent that Enhances CAR Activity

In another aspect, the CAR-expressing cell described herein can further express another agent, e.g., an agent that enhances the activity or fitness of a CAR-expressing cell.

For example, in one embodiment, the agent can be an agent which inhibits a molecule that modulates or regulates, e.g., inhibits, T cell function. In some embodiments, the molecule that modulates or regulates T cell function is an inhibitory molecule. Inhibitory molecules, e.g., PD1, can, in some embodiments, decrease the ability of a CAR-expressing cell to mount an immune effector response. Examples of inhibitory molecules include PD1, PD-L1, PD-L2, CTLA4, TIM3, CEACAM (e.g., CEACAM-1, CEACAM-3 and/or CEACAM-5), LAGS, VISTA, BTLA, TIGIT, LAIR1, CD160, 2B4, CD160, 2B4, CD80, CD86, B7-H3 (CD276), B7-H4 (VTCN1), HVEM (TNFRSF14 or CD270), KIR, A2aR, MHC class I, MHC class II, GAL9, adenosine, and TGF (e.g., TGF beta).

In one embodiment, an inhibitory nucleic acid, e.g., an inhibitory nucleic acid, e.g., a dsRNA, e.g., an siRNA or shRNA, a clustered regularly interspaced short palindromic repeats (CRISPR), a transcription-activator like effector nuclease (TALEN), or a zinc finger endonuclease (ZFN), e.g., as described herein, can be used to inhibit expression of a molecule that modulates or regulates, e.g., inhibits, T-cell function in the CAR-expressing cell. In an embodiment the agent is an shRNA, e.g., an shRNA described herein. In an embodiment, the agent that modulates or regulates, e.g., inhibits, T-cell function is inhibited within a CAR-expressing cell. For example, a dsRNA molecule that inhibits expression of a molecule that modulates or regulates, e.g., inhibits, T-cell function is linked to the nucleic acid that encodes a component, e.g., all of the components, of the CAR.

In one embodiment, the agent which inhibits an inhibitory molecule comprises a first polypeptide, e.g., an inhibitory molecule, associated with a second polypeptide that provides a positive signal to the cell, e.g., an intracellular signaling domain described herein. In one embodiment, the agent comprises a first polypeptide, e.g., of an inhibitory molecule such as PD1, PD-L1, PD-L2, CTLA4, TIM3, CEACAM (e.g., CEACAM-1, CEACAM-3 and/or CEACAM-5), LAGS, VISTA, BTLA, TIGIT, LAIR1, CD160, 2B4, CD160, 2B4, CD80, CD86, B7-H3 (CD276), B7-H4 (VTCN1), HVEM (TNFRSF14 or CD270), KIR, A2aR, MHC class I, MHC class II, GALS, adenosine, or TGF (e.g., TGF beta), or a fragment of any of these (e.g., at least a portion of an extracellular domain of any of these), and a second polypeptide which is an intracellular signaling domain described herein (e.g., comprising a costimulatory domain (e.g., 41BB, CD27 or CD28, e.g., as described herein) and/or a primary signaling domain (e.g., a CD3 zeta signaling domain described herein). In one embodiment, the agent comprises a first polypeptide of PD1 or a fragment thereof (e.g., at least a portion of an extracellular domain of PD1), and a second polypeptide of an intracellular signaling domain described herein (e.g., a CD28 signaling domain described herein and/or a CD3 zeta signaling domain described herein). PD1 is an inhibitory member of the CD28 family of receptors that also includes CD28, CTLA-4, ICOS, and BTLA. PD-1 is expressed on activated B cells, T cells and myeloid cells (Agata et al. 1996 Int. Immunol 8:765-75). Two ligands for PD1, PD-L1 and PD-L2 have been shown to downregulate T cell activation upon binding to PD1 (Freeman et a. 2000 J Exp Med 192:1027-34; Latchman et al. 2001 Nat Immunol 2:261-8; Carter et al. 2002 Eur J Immunol 32:634-43). PD-L1 is abundant in human cancers (Dong et al. 2003 J Mol Med 81:281-7; Blank et al. 2005 Cancer Immunol. Immunother 54:307-314; Konishi et al. 2004 Clin Cancer Res 10:5094). Immune suppression can be reversed by inhibiting the local interaction of PD1 with PD-L1.

In one embodiment, the agent comprises the extracellular domain (ECD) of an inhibitory molecule, e.g., Programmed Death 1 (PD1), can be fused to a transmembrane domain and intracellular signaling domains such as 41BB and CD3 zeta (also referred to herein as a PD1 CAR). In one embodiment, the PD1 CAR, when used in combinations with a CD19 CAR described herein, improves the persistence of the T cell. In one embodiment, the CAR is a PD1 CAR comprising the extracellular domain of PD1 indicated as underlined in SEQ ID NO: 121. In one embodiment, the PD1 CAR comprises the amino acid sequence of SEQ ID NO:121.

(SEQ ID NO: 121)

Malpvtalllplalllhaarppgwfldspdrpwnpptfspallvvtegdn
atftcsfsntsesfvlnwyrmspsnqtdklaafpedrsqpgqdcrfrvtq
lpngrdfhmsvvrarrndsgtylcgaislapkaqikeslraelrvterra
evptahpspsprpagqfqtlvtttpaprpptpaptiasqplslrpeacrp
aaggavhtrgldfacdiyiwaplagtcgvlllslvitlyckrgrkkllyi
fkqpfmrpvqttqeedgcscrfpeeeeggcelrvkfsrsadapaykqgqn
qlynelnlgrreeydvldkrrgrdpemggkprrknpqeglynelqkdkma
eayseigmkgerrrgkghdglyqglstatkdtydalhmqalppr.

In one embodiment, the PD1 CAR comprises the amino acid sequence provided below (SEQ ID NO:119).

(SEQ ID NO: 119)

pgwfldspdrpwnpptfspallvvtegdnatftcsfsntsesfvlnwyrm
spsnqtdklaafpedrsqpgqdcrfrvtqlpngrdfhmsvvrarrndsgt
ylcgaislapkaqikeslraelrvterraevptahpspsprpagqfqtlv
tttpaprpptpaptiasqplslrpeacrpaaggavhtrgldfacdiyiwa
plagtcgvlllslvitlyckrgrkkllyifkqpfmrpvqttqeedgcscr
fpeeeeggcelrvkfsrsadapaykqgqnqlynelnlgrreeydvldkrr
grdpemggkprrknpqeglynelqkdkmaeayseigmkgerrrgkghdgl
yqglstatkdtydalhmqalppr.

Tin one embodiment, the agent comprises a nucleic acid sequence encoding the PD1 CAR, e.g., the PD1 CAR described herein. In one embodiment, the nucleic acid sequence for the PD1 CAR is shown below, with the PD1 ECD underlined below in SEQ ID NO: 120

(SEQ ID NO: 120)

atggccctccctgtcactgccctgcttctccccctcgcactcctgctcca
cgccgctagaccacccggatggtttctggactctccggatcgcccgtgga
atcccccaaccttctcaccggcactcttggttgtgactgagggcgataat
gcgaccttcacgtgctcgttctccaacacctccgaatcattcgtgctgaa
ctggtaccgcatgagcccgtcaaaccagaccgacaagctcgccgcgtttc
cggaagatcggtcgcaaccgggacaggattgtcggttccgcgtgactcaa
ctgccgaatggcagagacttccacatgagcgtggtccgcgctaggcgaaa
cgactccgggacctacctgtgcggagccatctcgctggcgcctaaggccc
aaatcaaagagagcttgagggccgaactgagagtgaccgagcgcagagct
gaggtgccaactgcacatccatccccatcgcctcggcctgcggggcagtt
tcagaccctggtcacgaccactccggcgccgcgcccaccgactccggccc
caactatcgcgagccagcccctgtcgctgaggccggaagcatgccgccct
gccgccggaggtgctgtgcatacccggggattggacttcgcatgcgacat
ctacatttgggctcctctcgccggaacttgtggcgtgctccttctgtccc
tggtcatcaccctgtactgcaagcggggtcggaaaaagcttctgtacatt
ttcaagcagcccttcatgaggcccgtgcaaaccacccaggaggaggacgg
ttgctcctgccggttccccgaagaggaagaaggaggttgcgagctgcgcg
tgaagttctcccggagcgccgacgcccccgcctataagcagggccagaac
cagctgtacaacgaactgaacctgggacggcgggaagagtacgatgtgct
ggacaagcggcgcggccgggaccccgaaatgggcgggaagcctagaagaa
agaaccctcaggaaggcctgtataacgagctgcagaaggacaagatggcc
gaggcctactccgaaattgggatgaagggagagcggcggaggggaaaggg
gcacgacggcctgtaccaaggactgtccaccgccaccaaggacacatacg
atgccctgcacatgcaggcccttccccctcgc.

In another example, in one embodiment, the agent which enhances the activity of a CAR-expressing cell can be a costimulatory molecule or costimulatory molecule ligand. Examples of costimulatory molecules include MHC class I molecule, BTLA and a Toll ligand receptor, as well as OX40, CD27, CD28, CDS, ICAM-1, LFA-1 (CD11a/CD18), ICOS (CD278), and 4-1BB (CD137). Further examples of such costimulatory molecules include CDS, ICAM-1, GITR, BAFFR, HVEM (LIGHTR), SLAMF7, NKp80 (KLRF1), NKp44, NKp30, NKp46, CD160, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, NKG2D, NKG2C, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, and a ligand that specifically binds with CD83, e.g., as described herein. Examples of costimulatory molecule ligands include CD80, CD86, CD40L, ICOSL, CD70, OX40L, 4-1BBL, GITRL, and LIGHT. In embodiments, the costimulatory molecule ligand is a ligand for a costimulatory molecule different from the costimulatory molecule domain of the CAR. In embodiments, the costimulatory molecule ligand is a ligand for a costimulatory molecule that is the same as the costimulatory molecule domain of the CAR. In an embodiment, the costimulatory molecule ligand is 4-1BBL. In an embodiment, the costimulatory ligand is CD80 or CD86. In an embodiment, the costimulatory molecule ligand is CD70. In embodiments, a CAR-expressing immune effector cell described herein can be further engineered to express one or more additional costimulatory molecules or costimulatory molecule ligands.

Co-Expression of CAR with a Chemokine Receptor

In embodiments, the CAR-expressing cell described herein further comprises a chemokine receptor molecule. Transgenic expression of chemokine receptors CCR2b or CXCR2 in T cells enhances trafficking to CCL2- or CXCL1-secreting solid tumors including melanoma and neuroblastoma (Craddock et al., J Immunother. 2010 October; 33(8):780-8 and Kershaw et al., Hum Gene Ther. 2002 Nov. 1; 13(16):1971-80). Thus, without wishing to be bound by theory, it is believed that chemokine receptors expressed in CAR-expressing cells that recognize chemokines secreted by tumors, e.g., solid tumors, can improve homing of the CAR-expressing cell to the tumor, facilitate the infiltration of the CAR-expressing cell to the tumor, and enhances antitumor efficacy of the CAR-expressing cell. The chemokine receptor molecule can comprise a naturally occurring or recombinant chemokine receptor or a chemokine-binding fragment thereof. A chemokine receptor molecule suitable for expression in a CAR-expressing cell described herein include a CXC chemokine receptor (e.g., CXCR1, CXCR2, CXCR3, CXCR4, CXCR5, CXCR6, or CXCR7), a CC chemokine receptor (e.g., CCR1, CCR2, CCR3, CCR4, CCR5, CCR6, CCR7, CCR8, CCR9, CCR10, or CCR11), a CX3C chemokine receptor (e.g., CX3CR1), a XC chemokine receptor (e.g., XCR1), or a chemokine-binding fragment thereof. In one embodiment, the chemokine receptor molecule to be expressed with a CAR described herein is selected based on the chemokine(s) secreted by the tumor. In one embodiment, the CAR-expressing cell described herein further comprises, e.g., expresses, a CCR2b receptor or a CXCR2 receptor. In an embodiment, the CAR described herein and the chemokine receptor molecule are on the same vector or are on two different vectors. In embodiments where the CAR described herein and the chemokine receptor molecule are on the same vector, the CAR and the chemokine receptor molecule are each under control of two different promoters or are under the control of the same promoter.

### Conditional Expression of Immune Response-Enhancing Agents

Also provided herein are compositions and methods for conditionally expressing an agent that enhances the immune response or activity of a CAR-expressing cell described herein.

In one aspect, the present disclosure features an immune effector cell that is engineered to constitutively express a CAR, also referred to herein as a nonconditional CAR. In one embodiment, a nonconditional CAR as described herein comprises an antigen binding domain that binds to a cancer associated antigen, e.g., CD19, CD10, CD20, CD22, CD34, CD123, FLT-3, or ROR1. In embodiments, the nonconditional CAR-expressing immune effector cell further comprises a conditionally-expressed agent that enhances the therapeutic efficacy, e.g., the immune response, of the CAR-expressing immune effector cell. In such embodiments, the expression of the conditionally expressed agent occurs upon activation of the nonconditional CAR-expressing immune effector cell, e.g., upon binding of the nonconditional CAR molecule to its target, e.g., a cancer associated antigen, e.g., CD19, CD10, CD20, CD22, CD34, CD123, FLT-3, or ROR1.

Immune response-enhancing agents as described herein can be characterized by one or more of the following: 1) targets or binds to a different cancer associated antigen than that targeted by the nonconditional CAR; 2) inhibits the expression or activity of an immune checkpoint or inhibitory molecule; and/or 3) activates the expression and/or secretion of a component that enhances immune response or activation of an immune effector cell. The immune response-enhancing agent can be a polypeptide or a nucleic acid, e.g., a nucleic acid that encodes a polypeptide that enhances immune response. Examples of conditionally expressed agents that enhance the immune response include, but are not limited to, an additional CAR (referred to as a conditional CAR); a TCR-based molecule (e.g., a TCR-CAR); an inhibitor of an immune checkpoint or an inhibitory molecule; and/or a cytokine. In embodiments, the conditional CAR binds to a different cancer associated antigen than that targeted by the nonconditional CAR. In embodiments, the inhibitor of an immune checkpoint or inhibitory molecule described herein is an antibody or antigen binding fragment thereof, an inhibitory nucleic acid (e.g., an siRNA or shRNA), or a small molecule that inhibits or decreases the activity of an immune checkpoint or inhibitory molecule selected from PD1, PD-L1, CTLA4, TIM3, CEACAM (e.g., CEACAM-1, CEACAM-3 and/or CEACAM-5), LAGS, VISTA, BTLA, TIGIT, LAIR1, CD160, 2B4, CD80, CD86, B7-H3 (CD276), B7-H4 (VTCN1), HVEM (TNFRSF14 or CD270), KIR, A2aR, MHC class I, MHC class II, GALS, adenosine, or TGF beta. In embodiments, the cytokine comprises IL-2, IL-7, IL-15, or IL-21, or functional fragments or derivatives thereof.

In embodiments, the immune effector cell comprises a nonconditional CAR and one or more conditional CARs, where the conditional CAR binds to a different cancer associated antigen than that targeted by the nonconditional CAR. By way of example, in one embodiment, an immune effector cell comprises a nonconditional CAR that binds to CD19 and one or more conditional CARs that bind to CD10, CD20, CD22, CD34, CD123, FLT-3, or ROR1, or a combination thereof. In another embodiment, an immune effector cell comprises a nonconditional CAR that binds to CD10, CD20, CD22, CD34, CD123, FLT-3, or ROR1 and a conditional CAR that binds to CD19.

Conditional expression of the agent that enhances the immune response upon activation of the CAR-expressing immune effector cell is achieved by operatively linking an activation-conditional control region to the agent that enhances the immune response (e.g., to a nucleic acid sequence encoding such an agent). In one embodiment, the activation conditional control region comprises a promoter sequence that initiates expression, e.g., transcription, of the operatively linked immune response enhancing agent upon activation of the immune effector cell. In one embodiment, the activation conditional control region comprises one or more regulatory sequences (e.g., a transcription factor binding sequence or site) that facilitate the initiation of expression upon activation of the immune effector cell. In embodiments, the activation-conditional control region comprises a promoter sequence and/or one or more transcription factor binding sequences from a promoter or regulatory sequence of a gene that is upregulated upon one or more of the following: immune effector cell (e.g., T cell) activation, T-cell differentiation, T-cell polarization, or helper T cell development. Examples of such genes include, but are not limited to, NFAT (nuclear factor of activated T cells), ATF2 (activating transcription factor 2), NF-□B (nuclear factor-□B), IL-2, IL-2 receptor (IL-2R), IL-3, GM-CSF, IL-4, IL-10, and IFN-γ.

In one embodiment, the activation-conditional control region comprises one or more, e.g., 1, 2, 3, 4, 5, 6, or more, NFAT binding sequences or sites. In embodiments, the NFAT-binding sequence in the promoter comprises (5′-GGAAA-3′) (SEQ ID NO: 1312), optionally situated in a longer consensus sequence of 5′ (A/T)GGAAA(A/N)(A/T/C)N 3′ (SEQ ID NO: 1313). In embodiments, the NFAT-binding sequence is a Kb-like sequence such as GGGACT (SEQ ID NO: 1314). (See, Gibson et al., The Journal of Immunology, 2007, 179: 3831-3840.)

In one embodiment, the activation-conditional control region further comprises an IL-2 promoter (or a minimal IL-2 promoter), an IL-2R promoter, an ATF2 promoter, or a NF-□B promoter, or any functional fragment or derivative thereof. In one embodiment, the activation-conditional control region comprises one or more NFAT-binding sequences, e.g., 3 or 6 NFAT-binding sequences, and an IL-2 promoter, e.g., an IL-2 minimal promoter. In one embodiment, the activation-conditional control region comprises the sequence of

(SEQ ID NO: 1315)

AGCTTGGATCCAAGAGGAAAATTTGTTTCATACAGAAGGCGTTAAGAGGA
AAATTTGTTTCATACAGAAGGCGTTAAGAGGAAAATTTGTTTCATACAGA
AGGCGTTCAAGCTTGTCGAC.

### Sources of Cells

Prior to expansion and genetic modification or other modification, a source of cells, e.g., T cells or natural killer (NK) cells, can be obtained from a subject. Examples of subjects include humans, monkeys, chimpanzees, dogs, cats, mice, rats, and transgenic species thereof. T cells can be obtained from a number of sources, including peripheral blood mononuclear cells, bone marrow, lymph node tissue, cord blood, thymus tissue, tissue from a site of infection, ascites, pleural effusion, spleen tissue, and tumors.

In embodiments, immune effector cells (e.g., a population of immune effector cells), e.g., T cells, are derived from (e.g., differentiated from) a stem cell, e.g., an embryonic stem cell or a pluripotent stem cell, e.g., an induced pluripotent stem cell (iPSC). In embodiments, the cells are autologous or allogeneic. In embodiments wherein the cells are allogeneic, the cells, e.g., derived from stem cells (e.g., iPSCs), are modified to reduce their alloreactivity. For example, the cells can be modified to reduce alloreactivity, e.g., by modifying (e.g., disrupting) their T cell receptor. In embodiments, a site specific nuclease can be used to disrupt the T cell receptor, e.g., after T-cell differentiation. In other examples, cells, e.g., T cells derived from iPSCs, can be generated from virus-specific T cells, which are less likely to cause graft-versus-host disease because of their recognition of a pathogen-derived antigen. In yet other examples, alloreactivity can be reduced, e.g., minimized, by generating iPSCs from common HLA haplotypes such that they are histocompatible with matched, unrelated recipient subjects. In yet other examples, alloreactivity can be reduced, e.g., minimized, by repressing HLA expression through genetic modification. For example, T cells derived from iPSCs can be processed as described in, e.g., Themeli et al. Nat. Biotechnol. 31.10(2013):928-35, incorporated herein by reference. In some examples, immune effector cells, e.g., T cells, derived from stem cells, can be processed/generated using methods described in WO2014/165707, incorporated herein by reference.

In certain aspects of the present disclosure, immune effector cells, e.g., T cells, can be obtained from a unit of blood collected from a subject using any number of techniques known to the skilled artisan, such as Ficoll™ separation. In one aspect, cells from the circulating blood of an individual are obtained by apheresis. The apheresis product typically contains lymphocytes, including T cells, monocytes, granulocytes, B cells, other nucleated white blood cells, red blood cells, and platelets. In one aspect, the cells collected by apheresis may be washed to remove the plasma fraction and, optionally, to place the cells in an appropriate buffer or media for subsequent processing steps. In one embodiment, the cells are washed with phosphate buffered saline (PBS). In an alternative embodiment, the wash solution lacks calcium and may lack magnesium or may lack many if not all divalent cations.

Initial activation steps in the absence of calcium can lead to magnified activation. As those of ordinary skill in the art would readily appreciate a washing step may be accomplished by methods known to those in the art, such as by using a semi-automated “flow-through” centrifuge (for example, the Cobe 2991 cell processor, the Baxter CytoMate, or the Haemonetics Cell Saver 5) according to the manufacturer's instructions. After washing, the cells may be resuspended in a variety of biocompatible buffers, such as, for example, Ca-free, Mg-free PBS, PlasmaLyte A, or other saline solution with or without buffer. Alternatively, the undesirable components of the apheresis sample may be removed and the cells directly resuspended in culture media.

It is recognized that the methods of the application can utilize culture media conditions comprising 5% or less, for example 2%, human AB serum, and employ known culture media conditions and compositions, for example those described in Smith et al., “Ex vivo expansion of human T cells for adoptive immunotherapy using the novel Xeno-free CTS Immune Cell Serum Replacement” Clinical & Translational Immunology (2015) 4, e31; doi:10.1038/cti.2014.31.

In one aspect, T cells are isolated from peripheral blood lymphocytes by lysing the red blood cells and depleting the monocytes, for example, by centrifugation through a PERCOLL™ gradient or by counterflow centrifugal elutriation.

The methods described herein can include, e.g., selection of a specific subpopulation of immune effector cells, e.g., T cells, that are a T regulatory cell-depleted population, CD25+ depleted cells, using, e.g., a negative selection technique, e.g., described herein. In some embodiments, the population of T regulatory depleted cells contains less than 30%, 25%, 20%, 15%, 10%, 5%, 4%, 3%, 2%, 1% of CD25+ cells.

In one embodiment, T regulatory cells, e.g., CD25+ T cells, are removed from the population using an anti-CD25 antibody, or fragment thereof, or a CD25-binding ligand, IL-2. In one embodiment, the anti-CD25 antibody, or fragment thereof, or CD25-binding ligand is conjugated to a substrate, e.g., a bead, or is otherwise coated on a substrate, e.g., a bead. In one embodiment, the anti-CD25 antibody, or fragment thereof, is conjugated to a substrate as described herein.

In one embodiment, the T regulatory cells, e.g., CD25+ T cells, are removed from the population using CD25 depletion reagent from Miltenyi™. In one embodiment, the ratio of cells to CD25 depletion reagent is 1e7 cells to 20 uL, or 1e7 cells to 15 uL, or 1e7 cells to 10 uL, or 1e7 cells to 5 uL, or 1e7 cells to 2.5 uL, or 1e7 cells to 1.25 uL. In one embodiment, e.g., for T regulatory cells, e.g., CD25+ depletion, greater than 500 million cells/ml is used. In a further aspect, a concentration of cells of 600, 700, 800, or 900 million cells/ml is used.

In one embodiment, the population of immune effector cells to be depleted includes about 6×10⁹ CD25+ T cells. In other aspects, the population of immune effector cells to be depleted include about 1×10⁹ to 1×10¹⁰ CD25+ T cell, and any integer value in between. In one embodiment, the resulting population T regulatory depleted cells has 2×10⁹ T regulatory cells, e.g., CD25+ cells, or less (e.g., 1×10⁹, 5×10⁸, 1×10⁸, 5×10⁷, 1×10⁷, or less CD25+ cells).

In one embodiment, the T regulatory cells, e.g., CD25+ cells, are removed from the population using the CliniMAC system with a depletion tubing set, such as, e.g., tubing 162-01. In one embodiment, the CliniMAC system is run on a depletion setting such as, e.g., DEPLETION2.1.

Without wishing to be bound by a particular theory, decreasing the level of negative regulators of immune cells (e.g., decreasing the number of unwanted immune cells, e.g., T_{REG} cells), in a subject prior to apheresis or during manufacturing of a CAR-expressing cell product significantly reduces the risk of subject relapse. For example, methods of depleting T_{REG} cells are known in the art. Methods of decreasing T_{REG} cells include, but are not limited to, cyclophosphamide, anti-GITR antibody (an anti-GITR antibody described herein), CD25-depletion, mTOR inhibitor, and combinations thereof.

In some embodiments, the manufacturing methods comprise reducing the number of (e.g., depleting) T_{REG} cells prior to manufacturing of the CAR-expressing cell. For example, manufacturing methods comprise contacting the sample, e.g., the apheresis sample, with an anti-GITR antibody and/or an anti-CD25 antibody (or fragment thereof, or a CD25-binding ligand), e.g., to deplete T_{REG} cells prior to manufacturing of the CAR-expressing cell (e.g., T cell, NK cell) product.

Without wishing to be bound by a particular theory, decreasing the level of negative regulators of immune cells (e.g., decreasing the number of unwanted immune cells, e.g., T_{REG} cells), in a subject prior to apheresis or during manufacturing of a CAR-expressing cell product can reduce the risk of a T_{REG} relapse. In an embodiment, a subject is pre-treated with one or more therapies that reduce T_{REG} cells prior to collection of cells for CAR-expressing cell product manufacturing, thereby reducing the risk of subject relapse to CAR-expressing cell treatment. In an embodiment, methods of decreasing T_{REG} cells include, but are not limited to, administration to the subject of one or more of cyclophosphamide, anti-GITR antibody, CD25-depletion, or a combination thereof. In an embodiment, methods of decreasing T_{REG} cells include, but are not limited to, administration to the subject of one or more of cyclophosphamide, anti-GITR antibody, CD25-depletion, mTOR inhibitor, or a combination thereof. Administration of one or more of cyclophosphamide, anti-GITR antibody, CD25-depletion, or a combination thereof, can occur before, during or after an infusion of the CAR-expressing cell product. Administration of one or more of cyclophosphamide, anti-GITR antibody, CD25-depletion, mTOR inhibitor, or a combination thereof, can occur before, during or after an infusion of the CAR-expressing cell product.

In some embodiments, the manufacturing methods comprise reducing the number of (e.g., depleting) T_{REG} cells prior to manufacturing of the CAR-expressing cell. For example, manufacturing methods comprise contacting the sample, e.g., the apheresis sample, with an anti-GITR antibody and/or an anti-CD25 antibody (or fragment thereof, or a CD25-binding ligand), e.g., to deplete T_{REG} cells prior to manufacturing of the CAR-expressing cell (e.g., T cell, NK cell) product.

In an embodiment, a subject is pre-treated with one or more therapies that reduce T_{REG} cells prior to collection of cells for CAR-expressing cell product manufacturing, thereby reducing the risk of subject relapse to CAR-expressing cell treatment. In an embodiment, methods of decreasing T_{REG} cells include, but are not limited to, administration to the subject of one or more of cyclophosphamide, anti-GITR antibody, CD25-depletion, or a combination thereof. Administration of one or more of cyclophosphamide, anti-GITR antibody, CD25-depletion, or a combination thereof, can occur before, during or after an infusion of the CAR-expressing cell product.

In an embodiment, a subject is pre-treated with cyclophosphamide prior to collection of cells for CAR-expressing cell product manufacturing, thereby reducing the risk of subject relapse to CAR-expressing cell treatment. In an embodiment, a subject is pre-treated with an anti-GITR antibody prior to collection of cells for CAR-expressing cell product manufacturing, thereby reducing the risk of subject relapse to CAR-expressing cell treatment.

In one embodiment, the population of cells to be removed are neither the regulatory T cells or tumor cells, but cells that otherwise negatively affect the expansion and/or function of CART cells, e.g. cells expressing CD14, CD11b, CD33, CD15, or other markers expressed by potentially immune suppressive cells. In one embodiment, such cells are envisioned to be removed concurrently with regulatory T cells and/or tumor cells, or following said depletion, or in another order.

The methods described herein can include more than one selection step, e.g., more than one depletion step. Enrichment of a T cell population by negative selection can be accomplished, e.g., with a combination of antibodies directed to surface markers unique to the negatively selected cells. One method is cell sorting and/or selection via negative magnetic immunoadherence or flow cytometry that uses a cocktail of monoclonal antibodies directed to cell surface markers present on the cells negatively selected. For example, to enrich for CD4+ cells by negative selection, a monoclonal antibody cocktail can include antibodies to CD14, CD20, CD11b, CD16, HLA-DR, and CD8.

The methods described herein can further include removing cells from the population which express a tumor antigen, e.g., a tumor antigen that does not comprise CD25, e.g., CD19, CD30, CD38, CD123, CD20, CD14 or CD11b, to thereby provide a population of T regulatory depleted, e.g., CD25+ depleted, and tumor antigen depleted cells that are suitable for expression of a CAR, e.g., a CAR described herein. In one embodiment, tumor antigen expressing cells are removed simultaneously with the T regulatory, e.g., CD25+ cells. For example, an anti-CD25 antibody, or fragment thereof, and an anti-tumor antigen antibody, or fragment thereof, can be attached to the same substrate, e.g., bead, which can be used to remove the cells or an anti-CD25 antibody, or fragment thereof, or the anti-tumor antigen antibody, or fragment thereof, can be attached to separate beads, a mixture of which can be used to remove the cells. In other embodiments, the removal of T regulatory cells, e.g., CD25+ cells, and the removal of the tumor antigen expressing cells is sequential, and can occur, e.g., in either order.

Also provided are methods that include removing cells from the population which express a check point inhibitor, e.g., a check point inhibitor described herein, e.g., one or more of PD1+ cells, LAG3+ cells, and TIM3+ cells, to thereby provide a population of T regulatory depleted, e.g., CD25+ depleted cells, and check point inhibitor depleted cells, e.g., PD1+, LAG3+ and/or TIM3+ depleted cells. Exemplary check point inhibitors include PD1, PD-L1, PD-L2, CTLA4, TIM3, CEACAM (e.g., CEACAM-1, CEACAM-3 and/or CEACAM-5), LAG3, VISTA, BTLA, TIGIT, LAIR1, CD160, 2B4, CD80, CD86, B7-H3 (CD276), B7-H4 (VTCN1), HVEM (TNFRSF14 or CD270), KIR, A2aR, MHC class I, MHC class II, GALS, adenosine, and TGF (e.g., TGF beta), e.g., as described herein. In one embodiment, check point inhibitor expressing cells are removed simultaneously with the T regulatory, e.g., CD25+ cells. For example, an anti-CD25 antibody, or fragment thereof, and an anti-check point inhibitor antibody, or fragment thereof, can be attached to the same bead which can be used to remove the cells, or an anti-CD25 antibody, or fragment thereof, and the anti-check point inhibitor antibody, or fragment thereof, can be attached to separate beads, a mixture of which can be used to remove the cells. In other embodiments, the removal of T regulatory cells, e.g., CD25+ cells, and the removal of the check point inhibitor expressing cells is sequential, and can occur, e.g., in either order.

Methods described herein can include a positive selection step. For example, T cells can be isolated by incubation with anti-CD3/anti-CD28 (e.g., 3×28)-conjugated beads, such as DYNABEADS® M-450 CD3/CD28 T, for a time period sufficient for positive selection of the desired T cells. In one aspect, the time period is about 30 minutes. In a further aspect, the time period ranges from 30 minutes to 36 hours or longer and all integer values there between. In a further aspect, the time period is at least 1, 2, 3, 4, 5, or 6 hours. In yet another aspect, the time period is 10 to 24 hours. In one aspect, the incubation time period is 24 hours. Longer incubation times may be used to isolate T cells in any situation where there are few T cells as compared to other cell types, such in isolating tumor infiltrating lymphocytes (TIL) from tumor tissue or from immunocompromised individuals. Further, use of longer incubation times can increase the efficiency of capture of CD8+ T cells. Thus, by simply shortening or lengthening the time T cells are allowed to bind to the CD3/CD28 beads and/or by increasing or decreasing the ratio of beads to T cells (as described further herein), subpopulations of T cells can be preferentially selected for or against at culture initiation or at other time points during the process. Additionally, by increasing or decreasing the ratio of anti-CD3 and/or anti-CD28 antibodies on the beads or other surface, subpopulations of T cells can be preferentially selected for or against at culture initiation or at other desired time points.

In one embodiment, a T cell population can be selected that expresses one or more of IFN-γ, TNFα, IL-17A, IL-2, IL-3, IL-4, GM-CSF, IL-10, IL-13, granzyme B, and perforin, or other appropriate molecules, e.g., other cytokines. Methods for screening for cell expression can be determined, e.g., by the methods described in PCT Publication No.: WO 2013/126712.

For isolation of a desired population of cells by positive or negative selection, the concentration of cells and surface (e.g., particles such as beads) can be varied. In certain aspects, it may be desirable to significantly decrease the volume in which beads and cells are mixed together (e.g., increase the concentration of cells), to ensure maximum contact of cells and beads. For example, in one aspect, a concentration of about 10 billion cells/ml, 9 billion/ml, 8 billion/ml, 7 billion/ml, 6 billion/ml, or 5 billion/ml is used. In one aspect, a concentration of 1 billion cells/ml is used. In one aspect, a concentration of cells from 75, 80, 85, 90, 95, or 100 million cells/ml is used. In further aspects, concentrations of 125 or 150 million cells/ml can be used.

Using high concentrations can result in increased cell yield, cell activation, and cell expansion. Further, use of high cell concentrations allows more efficient capture of cells that may weakly express target antigens of interest, such as CD28-negative T cells, or from samples where there are many tumor cells present (e.g., leukemic blood, tumor tissue, etc.). Such populations of cells may have therapeutic value and would be desirable to obtain. For example, using high concentration of cells allows more efficient selection of CD8+ T cells that normally have weaker CD28 expression.

In a related aspect, it may be desirable to use lower concentrations of cells. By significantly diluting the mixture of T cells and surface (e.g., particles such as beads), interactions between the particles and cells is minimized. This selects for cells that express high amounts of desired antigens to be bound to the particles. For example, CD4+ T cells express higher levels of CD28 and are more efficiently captured than CD8+ T cells in dilute concentrations. In one aspect, the concentration of cells used is 5×10⁶/ml. In other aspects, the concentration used can be from about 1×10⁵/ml to 1×10⁶/ml, and any integer value in between.

In other aspects, the cells may be incubated on a rotator for varying lengths of time at varying speeds at either 2-10° C. or at room temperature.

T cells for stimulation can also be frozen after a washing step. Wishing not to be bound by theory, the freeze and subsequent thaw step provides a more uniform product by removing granulocytes and to some extent monocytes in the cell population. After the washing step that removes plasma and platelets, the cells may be suspended in a freezing solution. While many freezing solutions and parameters are known in the art and will be useful in this context, one method involves using PBS containing 20% DMSO and 8% human serum albumin, or culture media containing 10% Dextran 40 and 5% Dextrose, 20% Human Serum Albumin and 7.5% DMSO, or 31.25% Plasmalyte-A, 31.25% Dextrose 5%, 0.45% NaCl, 10% Dextran 40 and 5% Dextrose, 20% Human Serum Albumin, and 7.5% DMSO or other suitable cell freezing media containing for example, Hespan and PlasmaLyte A, the cells then are frozen to −80° C. at a rate of 1° per minute and stored in the vapor phase of a liquid nitrogen storage tank. Other methods of controlled freezing may be used as well as uncontrolled freezing immediately at −20° C. or in liquid nitrogen.

In certain aspects, cryopreserved cells are thawed and washed as described herein and allowed to rest for one hour at room temperature prior to activation using the methods of the present invention.

Also contemplated in the context of the invention is the collection of blood samples or apheresis product from a subject at a time period prior to when the expanded cells as described herein might be needed. As such, the source of the cells to be expanded can be collected at any time point necessary, and desired cells, such as T cells, isolated and frozen for later use in immune effector cell therapy for any number of diseases or conditions that would benefit from immune effector cell therapy, such as those described herein. In one aspect a blood sample or an apheresis is taken from a generally healthy subject. In certain aspects, a blood sample or an apheresis is taken from a generally healthy subject who is at risk of developing a disease, but who has not yet developed a disease, and the cells of interest are isolated and frozen for later use. In certain aspects, the T cells may be expanded, frozen, and used at a later time. In certain aspects, samples are collected from a patient shortly after diagnosis of a particular disease as described herein but prior to any treatments. In a further aspect, the cells are isolated from a blood sample or an apheresis from a subject prior to any number of relevant treatment modalities, including but not limited to treatment with agents such as natalizumab, efalizumab, antiviral agents, chemotherapy, radiation, immunosuppressive agents, such as cyclosporin, azathioprine, methotrexate, mycophenolate, and FK506, antibodies, or other immunoablative agents such as CAMPATH, anti-CD3 antibodies, cytoxan, fludarabine, cyclosporin, FK506, rapamycin, mycophenolic acid, steroids, FR901228, and irradiation.

In a further aspect of the present invention, T cells are obtained from a patient directly following treatment that leaves the subject with functional T cells. In this regard, it has been observed that following certain cancer treatments, in particular treatments with drugs that damage the immune system, shortly after treatment during the period when patients would normally be recovering from the treatment, the quality of T cells obtained may be optimal or improved for their ability to expand ex vivo. Likewise, following ex vivo manipulation using the methods described herein, these cells may be in a preferred state for enhanced engraftment and in vivo expansion. Thus, it is contemplated within the context of the present invention to collect blood cells, including T cells, dendritic cells, or other cells of the hematopoietic lineage, during this recovery phase. Further, in certain aspects, mobilization (for example, mobilization with GM-CSF) and conditioning regimens can be used to create a condition in a subject wherein repopulation, recirculation, regeneration, and/or expansion of particular cell types is favored, especially during a defined window of time following therapy. Illustrative cell types include T cells, B cells, dendritic cells, and other cells of the immune system.

In one embodiment, the immune effector cells expressing a CAR molecule, e.g., a CAR molecule described herein, are obtained from a subject that has received a low, immune enhancing dose of an mTOR inhibitor. In an embodiment, the population of immune effector cells, e.g., T cells, to be engineered to express a CAR, are harvested after a sufficient time, or after sufficient dosing of the low, immune enhancing, dose of an mTOR inhibitor, such that the level of PD1 negative immune effector cells, e.g., T cells, or the ratio of PD1 negative immune effector cells, e.g., T cells/PD1 positive immune effector cells, e.g., T cells, in the subject or harvested from the subject has been, at least transiently, increased.

In other embodiments, population of immune effector cells, e.g., T cells, which have, or will be engineered to express a CAR, can be treated ex vivo by contact with an amount of an mTOR inhibitor that increases the number of PD1 negative immune effector cells, e.g., T cells or increases the ratio of PD1 negative immune effector cells, e.g., T cells/PD1 positive immune effector cells, e.g., T cells.

In one embodiment, a T cell population is diacylglycerol kinase (DGK)-deficient. DGK-deficient cells include cells that do not express DGK RNA or protein, or have reduced or inhibited DGK activity. DGK-deficient cells can be generated by genetic approaches, e.g., administering RNA-interfering agents, e.g., siRNA, shRNA, miRNA, to reduce or prevent DGK expression. Alternatively, DGK-deficient cells can be generated by treatment with DGK inhibitors described herein.

In one embodiment, a T cell population is Ikaros-deficient. Ikaros-deficient cells include cells that do not express Ikaros RNA or protein, or have reduced or inhibited Ikaros activity, Ikaros-deficient cells can be generated by genetic approaches, e.g., administering RNA-interfering agents, e.g., siRNA, shRNA, miRNA, to reduce or prevent Ikaros expression. Alternatively, Ikaros-deficient cells can be generated by treatment with Ikaros inhibitors, e.g., lenalidomide.

In embodiments, a T cell population is DGK-deficient and Ikaros-deficient, e.g., does not express DGK and Ikaros, or has reduced or inhibited DGK and Ikaros activity. Such DGK and Ikaros-deficient cells can be generated by any of the methods described herein.

In an embodiment, the NK cells are obtained from the subject. In another embodiment, the NK cells are an NK cell line, e.g., NK-92 cell line (Conkwest).

### Allogeneic CAR

In embodiments described herein, the immune effector cell can be an allogeneic immune effector cell, e.g., T cell or NK cell. For example, the cell can be an allogeneic T cell, e.g., an allogeneic T cell lacking expression of a functional T cell receptor (TCR) and/or human leukocyte antigen (HLA), e.g., HLA class I and/or HLA class II.

A T cell lacking a functional TCR can be, e.g., engineered such that it does not express any functional TCR on its surface, engineered such that it does not express one or more subunits that comprise a functional TCR (e.g., engineered such that it does not express (or exhibits reduced expression) of TCR alpha, TCR beta, TCR gamma, TCR delta, TCR epsilon, and/or TCR zeta) or engineered such that it produces very little functional TCR on its surface (e.g., engineered such that it does not express (or exhibits reduced expression) of TCR alpha, TCR beta, TCR gamma, TCR delta, TCR epsilon, and/or TCR zeta). Alternatively, the T cell can express a substantially impaired TCR, e.g., by expression of mutated or truncated forms of one or more of the subunits of the TCR. The term “substantially impaired TCR” means that this TCR will not elicit an adverse immune reaction in a host.

A T cell described herein can be, e.g., engineered such that it does not express a functional HLA on its surface. For example, a T cell described herein, can be engineered such that cell surface expression HLA, e.g., HLA class 1 and/or HLA class II, is downregulated. In some embodiments, downregulation of HLA may be accomplished by reducing or eliminating expression of beta-2 microglobulin (B2M).

In some embodiments, the T cell can lack a functional TCR and a functional HLA, e.g., HLA class I and/or HLA class II.

Modified T cells that lack expression of a functional TCR and/or HLA can be obtained by any suitable means, including a knock out or knock down of one or more subunit of TCR or HLA. For example, the T cell can include a knock down of TCR and/or HLA using siRNA, shRNA, clustered regularly interspaced short palindromic repeats (CRISPR) transcription-activator like effector nuclease (TALEN), or zinc finger endonuclease (ZFN).

In some embodiments, the allogeneic cell can be a cell which does not express or expresses at low levels an inhibitory molecule, e.g. a cell engineered by any method described herein. For example, the cell can be a cell that does not express or expresses at low levels an inhibitory molecule, e.g., that can decrease the ability of a CAR-expressing cell to mount an immune effector response. Examples of inhibitory molecules include PD1, PD-L1, PD-L2, CTLA4, TIM3, CEACAM (e.g., CEACAM-1, CEACAM-3 and/or CEACAM-5), LAGS, VISTA, BTLA, TIGIT, LAIR1, CD160, 2B4, CD80, CD86, B7-H3 (CD276), B7-H4 (VTCN1), HVEM (TNFRSF14 or CD270), KIR, A2aR, MHC class I, MHC class II, GALS, adenosine, and TGF (e.g., TGF beta). Inhibition of an inhibitory molecule, e.g., by inhibition at the DNA, RNA or protein level, can optimize a CAR-expressing cell performance. In embodiments, an inhibitory nucleic acid, e.g., an inhibitory nucleic acid, e.g., a dsRNA, e.g., an siRNA or shRNA, a clustered regularly interspaced short palindromic repeats (CRISPR), a transcription-activator like effector nuclease (TALEN), or a zinc finger endonuclease (ZFN), e.g., as described herein, can be used.

siRNA and shRNA to Inhibit TCR or HLA

In some embodiments, TCR expression and/or HLA expression can be inhibited using siRNA or shRNA that targets a nucleic acid encoding a TCR and/or HLA, and/or an inhibitory molecule described herein (e.g., PD1, PD-L1, PD-L2, CTLA4, TIM3, CEACAM (e.g., CEACAM-1, CEACAM-3 and/or CEACAM-5), LAG3, VISTA, BTLA, TIGIT, LAIR1, CD160, 2B4, CD80, CD86, B7-H3 (CD276), B7-H4 (VTCN1), HVEM (TNFRSF14 or CD270), KIR, A2aR, MHC class I, MHC class II, GAL9, adenosine, and TGF beta), in a T cell.

Expression systems for siRNA and shRNAs, and exemplary shRNAs, are described, e.g., in paragraphs 649 and 650 of International Application WO2015/142675, filed Mar. 13, 2015, which is incorporated by reference in its entirety.

### CRISPR to Inhibit TCR or HLA

“CRISPR” or “CRISPR to TCR and/or HLA” or “CRISPR to inhibit TCR and/or HLA” as used herein refers to a set of clustered regularly interspaced short palindromic repeats, or a system comprising such a set of repeats. “Cas”, as used herein, refers to a CRISPR-associated protein. A “CRISPR/Cas” system refers to a system derived from CRISPR and Cas which can be used to silence or mutate a TCR and/or HLA gene, and/or an inhibitory molecule described herein (e.g., PD1, PD-L1, PD-L2, CTLA4, TIM3, CEACAM (e.g., CEACAM-1, CEACAM-3 and/or CEACAM-5), LAG3, VISTA, BTLA, TIGIT, LAIR1, CD160, 2B4, CD80, CD86, B7-H3 (CD276), B7-H4 (VTCN1), HVEM (TNFRSF14 or CD270), KIR, A2aR, MHC class I, MHC class II, GAL9, adenosine, and TGF beta).

The CRISPR/Cas system, and uses thereof, are described, e.g., in paragraphs 651-658 of International Application WO2015/142675, filed Mar. 13, 2015, which is incorporated by reference in its entirety.

TALEN to Inhibit TCR and/or HLA

“TALEN” or “TALEN to HLA and/or TCR” or “TALEN to inhibit HLA and/or TCR” refers to a transcription activator-like effector nuclease, an artificial nuclease which can be used to edit the HLA and/or TCR gene, and/or an inhibitory molecule described herein (e.g., PD1, PD-L1, PD-L2, CTLA4, TIM3, CEACAM (e.g., CEACAM-1, CEACAM-3 and/or CEACAM-5), LAG3, VISTA, BTLA, TIGIT, LAIR1, CD160, 2B4, CD80, CD86, B7-H3 (CD276), B7-H4 (VTCN1), HVEM (TNFRSF14 or CD270), KIR, A2aR, MHC class I, MHC class II, GAL9, adenosine, and TGF beta).

TALENs, TALEs, and uses thereof, are described, e.g., in paragraphs 659-665 of International Application WO2015/142675, filed Mar. 13, 2015, which is incorporated by reference in its entirety.

Zinc Finger Nuclease to Inhibit HLA and/or TCR

“ZFN” or “Zinc Finger Nuclease” or “ZFN to HLA and/or TCR” or “ZFN to inhibit HLA and/or TCR” refer to a zinc finger nuclease, an artificial nuclease which can be used to edit the HLA and/or TCR gene, and/or an inhibitory molecule described herein (e.g., PD1, PD-L1, PD-L2, CTLA4, TIM3, CEACAM (e.g., CEACAM-1, CEACAM-3 and/or CEACAM-5), LAG3, VISTA, BTLA, TIGIT, LAIR1, CD160, 2B4, CD80, CD86, B7-H3 (CD276), B7-H4 (VTCN1), HVEM (TNFRSF14 or CD270), KIR, A2aR, MHC class I, MHC class II, GAL9, adenosine, and TGF beta).

ZFNs, and uses thereof, are described, e.g., in paragraphs 666-671 of International Application WO2015/142675, filed Mar. 13, 2015, which is incorporated by reference in its entirety.

### Telomerase Expression

While not wishing to be bound by any particular theory, in some embodiments, a therapeutic T cell has short term persistence in a patient, due to shortened telomeres in the T cell; accordingly, transfection with a telomerase gene can lengthen the telomeres of the T cell and improve persistence of the T cell in the patient. See Carl June, “Adoptive T cell therapy for cancer in the clinic”, Journal of Clinical Investigation, 117:1466-1476 (2007). Thus, in an embodiment, an immune effector cell, e.g., a T cell, ectopically expresses a telomerase subunit, e.g., the catalytic subunit of telomerase, e.g., TERT, e.g., hTERT. In some aspects, this disclosure provides a method of producing a CAR-expressing cell, comprising contacting a cell with a nucleic acid encoding a telomerase subunit, e.g., the catalytic subunit of telomerase, e.g., TERT, e.g., hTERT. The cell may be contacted with the nucleic acid before, simultaneous with, or after being contacted with a construct encoding a CAR.

In one aspect, the disclosure features a method of making a population of immune effector cells (e.g., T cells or NK cells). In an embodiment, the method comprises: providing a population of immune effector cells (e.g., T cells or NK cells), contacting the population of immune effector cells with a nucleic acid encoding a CAR; and contacting the population of immune effector cells with a nucleic acid encoding a telomerase subunit, e.g., hTERT, under conditions that allow for CAR and telomerase expression.

In an embodiment, the nucleic acid encoding the telomerase subunit is DNA. In an embodiment, the nucleic acid encoding the telomerase subunit comprises a promoter capable of driving expression of the telomerase subunit.

In an embodiment, hTERT has the amino acid sequence of GenBank Protein ID AAC51724.1 (Meyerson et al., “hEST2, the Putative Human Telomerase Catalytic Subunit Gene, Is Up-Regulated in Tumor Cells and during Immortalization” Cell Volume 90, Issue 4, 22 Aug. 1997, Pages 785-795) as disclosed on pages 233-234 of International Application WO 2016/164731, filed Apr. 8, 2016, which is incorporated by reference in its entirety.

In an embodiment, the hTERT has a sequence at least 80%, 85%, 90%, 95%, 96\hat{ }, 97%, 98%, or 99% identical to the sequence of SEQ ID NO: 1332. In an embodiment, the hTERT has a sequence of SEQ ID NO: 1332. In an embodiment, the hTERT comprises a deletion (e.g., of no more than 5, 10, 15, 20, or 30 amino acids) at the N-terminus, the C-terminus, or both. In an embodiment, the hTERT comprises a transgenic amino acid sequence (e.g., of no more than 5, 10, 15, 20, or 30 amino acids) at the N-terminus, the C-terminus, or both.

In an embodiment, the hTERT is encoded by the nucleic acid sequence of GenBank Accession No. AF018167 (Meyerson et al., “hEST2, the Putative Human Telomerase Catalytic Subunit Gene, Is Up-Regulated in Tumor Cells and during Immortalization” Cell Volume 90, Issue 4, 22 August 1997, Pages 785-795) as disclosed on pages 234-235 of International Application WO 2016/164731, filed Apr. 8, 2016, which is incorporated by reference in its entirety.

In an embodiment, the hTERT is encoded by a nucleic acid having a sequence at least 80%, 85%, 90%, 95%, 96, 97%, 98%, or 99% identical to the sequence of SEQ ID NO: 1333. In an embodiment, the hTERT is encoded by a nucleic acid of SEQ ID NO: 1333.

### Activation and Expansion of Immune Effector Cells (e.g., T Cells)

Immune effector cells such as T cells may be activated and expanded generally using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041; and U.S. Patent Application Publication No. 20060121005.

The procedure for ex vivo expansion of hematopoietic stem and progenitor cells is described in U.S. Pat. No. 5,199,942, incorporated herein by reference, can be applied to the cells of the present invention. Other suitable methods are known in the art, therefore the present invention is not limited to any particular method of ex vivo expansion of the cells. Briefly, ex vivo culture and expansion of T cells can comprise: (1) collecting CD34+ hematopoietic stem and progenitor cells from a mammal from peripheral blood harvest or bone marrow explants; and (2) expanding such cells ex vivo. In addition to the cellular growth factors described in U.S. Pat. No. 5,199,942, other factors such as flt3-L, IL-1, IL-3 and c-kit ligand, can be used for culturing and expansion of the cells.

Generally, a population of immune effector cells may be expanded by contact with a surface having attached thereto an agent that stimulates a CD3/TCR complex associated signal and a ligand that stimulates a costimulatory molecule on the surface of the T cells. In particular, T cell populations may be stimulated as described herein, such as by contact with an anti-CD3 antibody, or antigen-binding fragment thereof, or an anti-CD2 antibody immobilized on a surface, or by contact with a protein kinase C activator (e.g., bryostatin) in conjunction with a calcium ionophore. For co-stimulation of an accessory molecule on the surface of the T cells, a ligand that binds the accessory molecule is used. For example, a population of T cells can be contacted with an anti-CD3 antibody and an anti-CD28 antibody, under conditions appropriate for stimulating proliferation of the T cells. To stimulate proliferation of either CD4+ T cells or CD8+ T cells, an anti-CD3 antibody and an anti-CD28 antibody may be used. Examples of an anti-CD28 antibody include 9.3, B-T3, XR-CD28 (Diaclone, Besancon, France) can be used as can other methods commonly known in the art (Berg et al., Transplant Proc. 30(8):3975-3977, 1998; Haanen et al., J. Exp. Med. 190(9):13191328, 1999; Garland et al., J. Immunol Meth. 227(1-2):53-63, 1999).

In some embodiments, immune effector cells (such as PBMCs or T cells) are expanded and stimulated by contacting the cells to one or both of an anti-CD3 antibody and IL-2. In embodiments, the cells are expanded without anti-CD3 or anti-CD28 beads.

In certain aspects, the primary stimulatory signal and the costimulatory signal for the T cell may be provided by different protocols. For example, the agents providing each signal may be in solution or coupled to a surface. When coupled to a surface, the agents may be coupled to the same surface (i.e., in “cis” formation) or to separate surfaces (i.e., in “trans” formation). Alternatively, one agent may be coupled to a surface and the other agent in solution. In one aspect, the agent providing the costimulatory signal is bound to a cell surface and the agent providing the primary activation signal is in solution or coupled to a surface. In certain aspects, both agents can be in solution. In one aspect, the agents may be in soluble form, and then cross-linked to a surface, such as a cell expressing Fc receptors or an antibody or other binding agent which will bind to the agents. In this regard, see for example, U.S. Patent Application Publication Nos. 20040101519 and 20060034810 for artificial antigen presenting cells (aAPCs) that are contemplated for use in activating and expanding T cells in the present invention.

In one aspect, the two agents are immobilized on beads, either on the same bead, i.e., “cis,” or to separate beads, i.e., “trans.” By way of example, the agent providing the primary activation signal is an anti-CD3 antibody or an antigen-binding fragment thereof and the agent providing the costimulatory signal is an anti-CD28 antibody or antigen-binding fragment thereof; and both agents are co-immobilized to the same bead in equivalent molecular amounts. In one aspect, a 1:1 ratio of each antibody bound to the beads for CD4+ T cell expansion and T cell growth is used. In certain aspects, a ratio of anti CD3:CD28 antibodies bound to the beads is used such that an increase in T cell expansion is observed as compared to the expansion observed using a ratio of 1:1. In one particular aspect an increase of from about 1 to about 3 fold is observed as compared to the expansion observed using a ratio of 1:1. In one aspect, the ratio of CD3:CD28 antibody bound to the beads ranges from 100:1 to 1:100 and all integer values there between. In one aspect, more anti-CD28 antibody is bound to the particles than anti-CD3 antibody, i.e., the ratio of CD3:CD28 is less than one. In certain aspects, the ratio of anti CD28 antibody to anti CD3 antibody bound to the beads is greater than 2:1. In one particular aspect, a 1:100 CD3:CD28 ratio of antibody bound to beads is used. In one aspect, a 1:75 CD3:CD28 ratio of antibody bound to beads is used. In a further aspect, a 1:50 CD3:CD28 ratio of antibody bound to beads is used. In one aspect, a 1:30 CD3:CD28 ratio of antibody bound to beads is used. In one aspect, a 1:10 CD3:CD28 ratio of antibody bound to beads is used. In one aspect, a 1:3 CD3:CD28 ratio of antibody bound to the beads is used. In yet one aspect, a 3:1 CD3:CD28 ratio of antibody bound to the beads is used.

Ratios of particles to cells from 1:500 to 500:1 and any integer values in between may be used to stimulate T cells or other target cells. As those of ordinary skill in the art can readily appreciate, the ratio of particles to cells may depend on particle size relative to the target cell. For example, small sized beads could only bind a few cells, while larger beads could bind many. In certain aspects the ratio of cells to particles ranges from 1:100 to 100:1 and any integer values in-between and in further aspects the ratio comprises 1:9 to 9:1 and any integer values in between, can also be used to stimulate T cells. The ratio of anti-CD3- and anti-CD28-coupled particles to T cells that result in T cell stimulation can vary as noted above, however certain suitable values include 1:100, 1:50, 1:40, 1:30, 1:20, 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, 10:1, and 15:1 with one suitable ratio being at least 1:1 particles per T cell. In one aspect, a ratio of particles to cells of 1:1 or less is used. In one particular aspect, a suitable particle: cell ratio is 1:5. In further aspects, the ratio of particles to cells can be varied depending on the day of stimulation. For example, in one aspect, the ratio of particles to cells is from 1:1 to 10:1 on the first day and additional particles are added to the cells every day or every other day thereafter for up to 10 days, at final ratios of from 1:1 to 1:10 (based on cell counts on the day of addition). In one particular aspect, the ratio of particles to cells is 1:1 on the first day of stimulation and adjusted to 1:5 on the third and fifth days of stimulation. In one aspect, particles are added on a daily or every other day basis to a final ratio of 1:1 on the first day, and 1:5 on the third and fifth days of stimulation. In one aspect, the ratio of particles to cells is 2:1 on the first day of stimulation and adjusted to 1:10 on the third and fifth days of stimulation. In one aspect, particles are added on a daily or every other day basis to a final ratio of 1:1 on the first day, and 1:10 on the third and fifth days of stimulation. One of skill in the art will appreciate that a variety of other ratios may be suitable for use in the present invention. In particular, ratios will vary depending on particle size and on cell size and type. In one aspect, the most typical ratios for use are in the neighborhood of 1:1, 2:1 and 3:1 on the first day.

In further aspects of the present invention, the cells, such as T cells, are combined with agent-coated beads, the beads and the cells are subsequently separated, and then the cells are cultured. In an alternative aspect, prior to culture, the agent-coated beads and cells are not separated but are cultured together. In a further aspect, the beads and cells are first concentrated by application of a force, such as a magnetic force, resulting in increased ligation of cell surface markers, thereby inducing cell stimulation.

By way of example, cell surface proteins may be ligated by allowing paramagnetic beads to which anti-CD3 and anti-CD28 are attached (3×28 beads) to contact the T cells. In one aspect the cells (for example, 10⁴ to 10⁹ T cells) and beads (for example, DYNABEADS® M-450 CD3/CD28 T paramagnetic beads at a ratio of 1:1) are combined in a buffer, for example PBS (without divalent cations such as, calcium and magnesium). Again, those of ordinary skill in the art can readily appreciate any cell concentration may be used. For example, the target cell may be very rare in the sample and comprise only 0.01% of the sample or the entire sample (i.e., 100%) may comprise the target cell of interest. Accordingly, any cell number is within the context of the present invention. In certain aspects, it may be desirable to significantly decrease the volume in which particles and cells are mixed together (i.e., increase the concentration of cells), to ensure maximum contact of cells and particles. For example, in one aspect, a concentration of about 10 billion cells/ml, 9 billion/ml, 8 billion/ml, 7 billion/ml, 6 billion/ml, or 5 billion/ml or 2 billion cells/ml is used. In one aspect, greater than 100 million cells/ml is used. In a further aspect, a concentration of cells of 10, 15, 20, 25, 30, 35, 40, 45, or 50 million cells/ml is used. In yet one aspect, a concentration of cells from 75, 80, 85, 90, 95, or 100 million cells/ml is used. In further aspects, concentrations of 125 or 150 million cells/ml can be used. Using high concentrations can result in increased cell yield, cell activation, and cell expansion. Further, use of high cell concentrations allows more efficient capture of cells that may weakly express target antigens of interest, such as CD28-negative T cells. Such populations of cells may have therapeutic value and would be desirable to obtain in certain aspects. For example, using high concentration of cells allows more efficient selection of CD8+ T cells that normally have weaker CD28 expression.

In one embodiment, cells transduced with a nucleic acid encoding a CAR, e.g., a CAR described herein, are expanded, e.g., by a method described herein. In one embodiment, the cells are expanded in culture for a period of several hours (e.g., about 2, 3, 4, 5, 6, 7, 8, 9, 10, 15, 18, 21 hours) to about 14 days (e.g., 1, 2, 3, 4, 5, 6, 7, 8, 9, 10, 11, 12, 13 or 14 days). In one embodiment, the cells are expanded for a period of 4 to 9 days. In one embodiment, the cells are expanded for a period of 8 days or less, e.g., 7, 6 or 5 days. In one embodiment, the cells, e.g., a CAR cell described herein, are expanded in culture for 5 days, and the resulting cells are more potent than the same cells expanded in culture for 9 days under the same culture conditions. Potency can be defined, e.g., by various T cell functions, e.g. proliferation, target cell killing, cytokine production, activation, migration, or combinations thereof. In one embodiment, the cells, e.g., a CD19 CAR cell described herein, expanded for 5 days show at least a one, two, three or four fold increase in cells doublings upon antigen stimulation as compared to the same cells expanded in culture for 9 days under the same culture conditions. In one embodiment, the cells, e.g., the cells expressing a CAR described herein, are expanded in culture for 5 days, and the resulting cells exhibit higher proinflammatory cytokine production, e.g., IFN-γ and/or GM-CSF levels, as compared to the same cells expanded in culture for 9 days under the same culture conditions. In one embodiment, the cells, e.g., a CAR cell described herein, expanded for 5 days show at least a one, two, three, four, five, ten fold or more increase in pg/ml of proinflammatory cytokine production, e.g., IFN-γ and/or GM-CSF levels, as compared to the same cells expanded in culture for 9 days under the same culture conditions.

In one aspect of the present invention, the mixture may be cultured for several hours (about 3 hours) to about 14 days or any hourly integer value in between. In one aspect, the mixture may be cultured for 21 days. In one aspect of the invention the beads and the T cells are cultured together for about eight days. In one aspect, the beads and T cells are cultured together for 2-3 days.

Several cycles of stimulation may also be desired such that culture time of T cells can be 60 days or more. Conditions appropriate for T cell culture include an appropriate media (e.g., Minimal Essential Media or RPMI Media 1640 or, X-vivo 15, (Lonza)) that may contain factors necessary for proliferation and viability, including serum (e.g., fetal bovine or human serum), interleukin-2 (IL-2), insulin, IFN-γ, IL-4, IL-7, GM-CSF, IL-10, IL-12, IL-15, TGFβ, and TNF-α or any other additives for the growth of cells known to the skilled artisan. Other additives for the growth of cells include, but are not limited to, surfactant, plasmanate, and reducing agents such as N-acetyl-cysteine and 2-mercaptoethanol. Media can include RPMI 1640, AIM-V, DMEM, MEM, α-MEM, F-12, X-Vivo 15, and X-Vivo 20, Optimizer, with added amino acids, sodium pyruvate, and vitamins, either serum-free or supplemented with an appropriate amount of serum (or plasma) or a defined set of hormones, and/or an amount of cytokine(s) sufficient for the growth and expansion of T cells. Antibiotics, e.g., penicillin and streptomycin, are included only in experimental cultures, not in cultures of cells that are to be infused into a subject. The target cells are maintained under conditions necessary to support growth, for example, an appropriate temperature (e.g., 37° C.) and atmosphere (e.g., air plus 5% CO₂).

In one embodiment, the cells are expanded in an appropriate media (e.g., media described herein) that includes one or more interleukin that result in at least a 200-fold (e.g., 200-fold, 250-fold, 300-fold, 350-fold) increase in cells over a 14 day expansion period, e.g., as measured by a method described herein such as flow cytometry. In one embodiment, the cells are expanded in the presence IL-15 and/or IL-7 (e.g., IL-15 and IL-7).

In some embodiments a CAR-expressing cell described herein (e.g., a T cell such as a CD4+ T cell or a CD8+ T cell) is contacted with a composition comprising a interleukin-15 (IL-15) polypeptide, a interleukin-15 receptor alpha (IL-15Ra) polypeptide, or a combination of both a IL-15 polypeptide and a IL-15Ra polypeptide e.g., hetIL-15, during the manufacturing of the CAR-expressing cell, e.g., ex vivo. In embodiments, a CAR-expressing cell described herein is contacted with a composition comprising a IL-15 polypeptide during the manufacturing of the CAR-expressing cell, e.g., ex vivo. In embodiments, a CAR-expressing cell described herein is contacted with a composition comprising a combination of both a IL-15 polypeptide and a IL-15 Ra polypeptide during the manufacturing of the CAR-expressing cell, e.g., ex vivo. In embodiments, a CAR-expressing cell described herein is contacted with a composition comprising hetIL-15 during the manufacturing of the CAR-expressing cell, e.g., ex vivo.

In one embodiment the CAR-expressing cell (e.g., a T cell or NK cell) described herein is contacted with a composition comprising hetIL-15 during ex vivo expansion. In an embodiment, the CAR-expressing cell described herein is contacted with a composition comprising an IL-15 polypeptide during ex vivo expansion. In an embodiment, the CAR-expressing cell described herein is contacted with a composition comprising both an IL-15 polypeptide and an IL-15Ra polypeptide during ex vivo expansion. In one embodiment the contacting results in the survival and proliferation of a lymphocyte subpopulation, e.g., CD8+ T cells.

In an embodiment, the method of making disclosed herein further comprises contacting the population of immune effector cells (e.g., T cells or NK cells) with a nucleic acid encoding a telomerase subunit, e.g., hTERT. The nucleic acid encoding the telomerase subunit can be DNA.

T cells that have been exposed to varied stimulation times may exhibit different characteristics. For example, typical blood or apheresed peripheral blood mononuclear cell products have a helper T cell population (TH, CD4+) that is greater than the cytotoxic or suppressor T cell population (TC, CD8+). Ex vivo expansion of T cells by stimulating CD3 and CD28 receptors produces a population of T cells that prior to about days 8-9 consists predominately of TH cells, while after about days 8-9, the population of T cells comprises an increasingly greater population of TC cells. Accordingly, depending on the purpose of treatment, infusing a subject with a T cell population comprising predominately of TH cells may be advantageous. Similarly, if an antigen-specific subset of TC cells has been isolated it may be beneficial to expand this subset to a greater degree.

Further, in addition to CD4 and CD8 markers, other phenotypic markers vary significantly, but in large part, reproducibly during the course of the cell expansion process. Thus, such reproducibility enables the ability to tailor an activated T cell product for specific purposes.

Once a CAR, e.g., CD19 CAR is constructed, various assays can be used to evaluate the activity of the molecule, such as but not limited to, the ability to expand T cells following antigen stimulation, sustain T cell expansion in the absence of re-stimulation, and anti-cancer activities in appropriate in vitro and animal models. Assays to evaluate the effects of a CAR, e.g., CD19 CAR are described in further detail below.

Western blot analysis of CAR expression in primary T cells can be used to detect the presence of monomers and dimers, e.g., as described in paragraph 695 of International Application WO2015/142675, filed Mar. 13, 2015, which is herein incorporated by reference in its entirety.

In vitro expansion of CARP T cells following antigen stimulation can be measured by flow cytometry. For example, a mixture of CD4⁺ and CD8⁺ T cells are stimulated with αCD3/αCD28 beads followed by transduction with lentiviral vectors expressing GFP under the control of the promoters to be analyzed. Exemplary promoters include the CMV IE gene, EF-1α, ubiquitin C, or phosphoglycerokinase (PGK) promoters. GFP fluorescence is evaluated on day 6 of culture in the CD4⁺ and/or CD8⁺ T cell subsets by flow cytometry. See, e.g., Milone et al., Molecular Therapy 17(8): 1453-1464 (2009). Alternatively, a mixture of CD4⁺ and CD8⁺ T cells are stimulated with αCD3/αCD28 coated magnetic beads on day 0, and transduced with CAR on day 1 using a bicistronic lentiviral vector expressing CAR along with eGFP using a 2A ribosomal skipping sequence. Cultures are re-stimulated with either CD19+K562 cells (K562-CD19), wild-type K562 cells (K562 wild type) or K562 cells expressing hCD32 and 4-1BBL in the presence of anti-CD3 and anti-CD28 antibody (K562-BBL-3/28) following washing. Exogenous IL-2 is added to the cultures every other day at 100 IU/ml. GFP⁺ T cells are enumerated by flow cytometry using bead-based counting. See, e.g., Milone et al., Molecular Therapy 17(8): 1453-1464 (2009).

Sustained CAR⁺ T cell expansion in the absence of re-stimulation can also be measured. See, e.g., Milone et al., Molecular Therapy 17(8): 1453-1464 (2009). Briefly, mean T cell volume (fl) is measured on day 8 of culture using a Coulter Multisizer particle counter, a Nexcelom Cellometer Vision, or Millipore Scepter following stimulation with αCD3/αCD28 coated magnetic beads on day 0, and transduction with the indicated CAR on day 1.

Animal models can also be used to measure a CAR-expressing cell activity, e.g., as described in paragraph 698 of International Application WO2015/142675, filed Mar. 13, 2015, which is herein incorporated by reference in its entirety.

Dose dependent CAR treatment response can be evaluated, e.g., as described in paragraph 699 of International Application WO2015/142675, filed Mar. 13, 2015, which is herein incorporated by reference in its entirety. Assessment of cell proliferation and cytokine production has been previously described, e.g., as described in paragraph 700 of International Application WO2015/142675, filed Mar. 13, 2015, which is herein incorporated by reference in its entirety. Cytotoxicity can be assessed by a standard ⁵¹Cr-release assay, e.g., as described in paragraph 701 of International Application WO2015/142675, filed Mar. 13, 2015, which is herein incorporated by reference in its entirety. Imaging technologies can be used to evaluate specific trafficking and proliferation of CARs in tumor-bearing animal models, e.g., as described in paragraph 702 of International Application WO2015/142675, filed Mar. 13, 2015, which is herein incorporated by reference in its entirety.

Other assays, including those described in the Example section herein as well as those that are known in the art can also be used to evaluate the CARs described herein.

Alternatively, or in combination to the methods disclosed herein, methods and compositions for one or more of detection and/or quantification of CAR-expressing cells (e.g., in vitro or in vivo (e.g., clinical monitoring)), immune cell expansion and/or activation, and/or CAR-specific selection, that involve the use of a CAR ligand, are disclosed. In one exemplary embodiment, the CAR ligand is an antibody that binds to the CAR molecule, e.g., binds to the extracellular antigen binding domain of CAR (e.g., an antibody that binds to the antigen binding domain, e.g., an anti-idiotypic antibody; or an antibody that binds to a constant region of the extracellular binding domain). In other embodiments, the CAR ligand is a CAR antigen molecule (e.g., a CAR antigen molecule as described herein).

In one aspect, a method for detecting and/or quantifying CAR-expressing cells is disclosed. For example, the CAR ligand can be used to detect and/or quantify CAR-expressing cells in vitro or in vivo (e.g., clinical monitoring of CAR-expressing cells in a patient, or dosing a patient). The method includes:
providing the CAR ligand (optionally, a labelled CAR ligand, e.g., a CAR ligand that includes a tag, a bead, a radioactive or fluorescent label);
acquiring the CAR-expressing cell (e.g., acquiring a sample containing CAR-expressing cells, such as a manufacturing sample or a clinical sample);
contacting the CAR-expressing cell with the CAR ligand under conditions where binding occurs, thereby detecting the level (e.g., amount) of the CAR-expressing cells present. Binding of the CAR-expressing cell with the CAR ligand can be detected using standard techniques such as FACS, ELISA and the like.

In another aspect, a method of expanding and/or activating cells (e.g., immune effector cells) is disclosed. The method includes:
providing a CAR-expressing cell (e.g., a first CAR-expressing cell or a transiently expressing CAR cell);
contacting said CAR-expressing cell with a CAR ligand, e.g., a CAR ligand as described herein), under conditions where immune cell expansion and/or proliferation occurs, thereby producing the activated and/or expanded cell population.

In certain embodiments, the CAR ligand is present on (e.g., is immobilized or attached to a substrate, e.g., a non-naturally occurring substrate). In some embodiments, the substrate is a non-cellular substrate. The non-cellular substrate can be a solid support chosen from, e.g., a plate (e.g., a microtiter plate), a membrane (e.g., a nitrocellulose membrane), a matrix, a chip or a bead. In embodiments, the CAR ligand is present in the substrate (e.g., on the substrate surface). The CAR ligand can be immobilized, attached, or associated covalently or non-covalently (e.g., cross-linked) to the substrate. In one embodiment, the CAR ligand is attached (e.g., covalently attached) to a bead. In the aforesaid embodiments, the immune cell population can be expanded in vitro or ex vivo. The method can further include culturing the population of immune cells in the presence of the ligand of the CAR molecule, e.g., using any of the methods described herein.

In other embodiments, the method of expanding and/or activating the cells further comprises addition of a second stimulatory molecule, e.g., CD28. For example, the CAR ligand and the second stimulatory molecule can be immobilized to a substrate, e.g., one or more beads, thereby providing increased cell expansion and/or activation.

In other embodiments, a method for selecting or enriching for a CAR expressing cell is provided. The method includes contacting the CAR expressing cell with a CAR ligand as described herein; and selecting the cell on the basis of binding of the CAR ligand.

In yet other embodiments, a method for depleting (e.g., reducing and/or killing) a CAR expressing cell is provided. The method includes contacting the CAR expressing cell with a CAR ligand as described herein; and targeting the cell on the basis of binding of the CAR ligand thereby reducing the number, and/or killing, the CAR-expressing cell. In one embodiment, the CAR ligand is coupled to a toxic agent (e.g., a toxin or a cell ablative drug). In another embodiment, the anti-idiotypic antibody can cause effector cell activity, e.g., ADCC or ADC activities.

Exemplary anti-CAR antibodies that can be used in the methods disclosed herein are described, e.g., in WO 2014/190273 and by Jena et al., “Chimeric Antigen Receptor (CAR)-Specific Monoclonal Antibody to Detect CD19-Specific T cells in Clinical Trials”, PLOS March 2013 8:3 e57838, the contents of which are incorporated by reference. In some aspects and embodiments, the compositions and methods herein are optimized for a specific subset of T cells, e.g., as described in US Serial No. PCT/US2015/043219 filed Jul. 31, 2015, the contents of which are incorporated herein by reference in their entirety. In some embodiments, the optimized subsets of T cells display an enhanced persistence compared to a control T cell, e.g., a T cell of a different type (e.g., CD8+ or CD4+) expressing the same construct.

In some embodiments, a CD4+ T cell comprises a CAR described herein, which CAR comprises an intracellular signaling domain suitable for (e.g., optimized for, e.g., leading to enhanced persistence in) a CD4+ T cell, e.g., an ICOS domain. In some embodiments, a CD8+ T cell comprises a CAR described herein, which CAR comprises an intracellular signaling domain suitable for (e.g., optimized for, e.g., leading to enhanced persistence of) a CD8+ T cell, e.g., a 4-1BB domain, a CD28 domain, or another costimulatory domain other than an ICOS domain. In some embodiments, the CAR described herein comprises an antigen binding domain described herein, e.g., a CAR comprising an antigen binding domain.

In an aspect, described herein is a method of treating a subject, e.g., a subject having cancer. The method includes administering to said subject, an effective amount of:
1) a CD4+ T cell comprising a CAR (the CARCD4+) comprising:
an antigen binding domain, e.g., an antigen binding domain described herein;
a transmembrane domain; and
an intracellular signaling domain, e.g., a first costimulatory domain, e.g., an ICOS domain; and
2) a CD8+ T cell comprising a CAR (the CARCD8+) comprising:
an antigen binding domain, e.g., an antigen binding domain described herein;
a transmembrane domain; and
an intracellular signaling domain, e.g., a second costimulatory domain, e.g., a 4-1BB domain, a CD28 domain, or another costimulatory domain other than an ICOS domain;
wherein the CARCD4+ and the CARCD8+ differ from one another.
Optionally, the method further includes administering:
3) a second CD8+ T cell comprising a CAR (the second CARCD8+) comprising:
an antigen binding domain, e.g., an antigen binding domain described herein;
a transmembrane domain; and
an intracellular signaling domain, wherein the second CARCD8+ comprises an intracellular signaling domain, e.g., a costimulatory signaling domain, not present on the CARCD8+, and, optionally, does not comprise an ICOS signaling domain.

### Methods of Manufacture/Production

In certain aspects, the disclosure provides a method of making a cell, comprising transducing an immune effector cell, e.g., a T cell or NK cell, with a vector as described herein, e.g., a vector encoding a CAR. In certain aspects, the disclosure provides a method of making a cell, comprising introducing a nucleic acid as described herein (e.g., a nucleic acid encoding a CAR) into an immune effector cell, e.g., a T cell or NK cell. In certain aspects, the disclosure provides a method of generating a population of RNA-engineered cells comprising introducing an in vitro transcribed RNA or synthetic RNA into a cell, where the RNA comprises a nucleic acid as described herein, e.g., a nucleic acid encoding a CAR.

In some embodiments, the methods of making disclosed herein further comprise contacting the population of cells, (e.g., CD19 CAR-expressing cells, CD20 CAR-expressing cells, CD22 CAR-expressing cells, B-cell inhibitor cells, or both of CD19 CAR-expressing cells and B-cell inhibitor cells), with a nucleic acid encoding a telomerase subunit, e.g., hTERT. The nucleic acid encoding the telomerase subunit can be DNA.

In some embodiments, the method of making disclosed herein further comprises culturing the population of cells, (e.g., a population of CAR-expressing cells, e.g., CD19 CAR-expressing cells), in serum comprising 2% hAB serum.

In some aspects, the present disclosure provides a method of evaluating suitability for manufacturing, e.g., high or low suitability for manufacturing (e.g., predicting high manufacturing success or low manufacturing success, e.g., manufacturing fail) of a CAR-expressing cell product, e.g., CAR19-expressing cell product sample (e.g., CTL019 or CTL119). The method comprises:
(1) acquiring a sample comprising immune effector cells (e.g., a whole blood sample, peripheral blood sample, or apheresis sample) from a patient having a cancer, e.g., NHL; and
(2) evaluating the suitability for manufacturing by determining, from the sample, one, two, three, four, five, six, seven, eight, nine or more (e.g., all) of:
   (i) complete blood count, e.g., complete blood count with differential;
   (ii) absolute lymphocyte count (ALC);
   (iii) absolute monocyte count (AMC);
   (iv) percent or number of lymphocytes;
   (v) percent or number of neutrophils;
   (vi) percent or number of CD3+CD45+ cells;
   (vii) percent or number of monocytes;
   (viii) percent or number of CD45 dim or CD45 negative cells;
   (ix) percent or number of CD15+ and/or CXCR2+ cells; or
   (x) percent or number of suppressive non-lymphoid cell, e.g., myeloid derived suppressor cells (MDSC);
   wherein low levels of (i), (ii), (iii), (iv), or (vi) or high levels of (v), (vii), (viii), (ix) or (x) are indicative of low suitability for manufacturing, or
   wherein high levels of (i), (ii), (iii), (iv), or (vi) or low levels of (v), (vii), (viii), (ix) or (x) are indicative of high suitability for manufacturing,
      thereby evaluating the suitability for manufacturing of the CAR-expressing cell product.

In some aspects, the present disclosure provides a method of evaluating a sample, or a method of manufacturing CAR-expressing cells, comprising:
(1) acquiring a sample comprising immune effector cells (e.g., a whole blood sample, peripheral blood sample, or apheresis sample) from a patient having a cancer, e.g., NHL; and
(2) evaluating one, two, three, four, five, six, seven, eight, nine or more (e.g., all) of:
   (i) complete blood count, e.g., complete blood count with differential;
   (ii) absolute lymphocyte count;
   (iii) absolute monocyte count;
   (iv) percent or number of lymphocytes;
   (v) percent or number of neutrophils;
   (vi) percent or number of CD3+CD45+ cells;
   (vii) percent or number of monocytes;
   (viii) percent or number of CD45 dim or CD45 negative cells;
   (ix) percent or number of CD15+ and/or CXCR2+ cells; or
   (x) percent or number of suppressive non-lymphoid cell, e.g., myeloid derived suppressor cells (MDSC); and

(3) optionally contacting the cell sample with a nucleic acid encoding CAR molecule, e.g., a CAR molecule described herein, e.g., a CD19 CAR.

In embodiments of any of the manufacturing or evaluating aspects herein, low levels of (i), (ii), (iii), (iv), or (vi) or high levels of (v) or (vii), (viii), (ix) or (x) are indicative of low suitability for manufacturing.

In embodiments of any of the manufacturing or evaluating aspects herein, high levels of (i), (ii), (iii), (iv), or (vi) or low levels of (v), (vii), (viii), (ix) or (x) are indicative of high suitability for manufacturing.

In embodiments of any of the manufacturing or evaluating aspects herein, the method comprises evaluating two of (i), (ii), (iii), (iv), (v), (vi), (vii), (viii), (ix) or (x). In embodiments, the method comprises evaluating three of (i), (ii), (iii), (iv), (v), (vi), (vii), (viii), (ix) or (x). In embodiments, the method comprises evaluating four of (i), (ii), (iii), (iv), (v), (vi), (vii), (viii), (ix) or (x). In embodiments, the method comprises evaluating five of (i), (ii), (iii), (iv), (v), (vi), (vii), (viii), (ix) or (x). In embodiments, the method comprises evaluating six of (i), (ii), (iii), (iv), (v), (vi), (vii), (viii), (ix) or (x). In embodiments, the method comprises evaluating seven of (i), (ii), (iii), (iv), (v), (vi), (vii), (viii), (ix) or (x). In embodiments, the method comprises evaluating eight of (i), (ii), (iii), (iv), (v), (vi), (vii), (viii), (ix) or (x). In embodiments, the method comprises evaluating nine of (i), (ii), (iii), (iv), (v), (vi), (vii), (viii), (ix) or (x). In embodiments, the method comprises evaluating all of (i), (ii), (iii), (iv), (v), (vi), (vii), (viii), (ix) or (x).

In embodiments of any of the manufacturing or evaluating aspects herein, wherein the absolute lymphocyte count is greater than or equal to 500/ul, the sample is suitable for manufacturing, e.g., the likelihood of manufacturing success is about 93%. In embodiments, wherein the absolute lymphocyte count is <500/ul, there is a reduced suitability for manufacturing, e.g., the likelihood of manufacturing success is about 65%. In embodiments, wherein the absolute lymphocyte count is <300/ul, there is a reduced suitability for manufacturing, e.g., the likelihood of manufacturing success is about 40%. In embodiments, wherein the absolute monocyte count is <500/ul, there is a reduced suitability for manufacturing. In embodiments, wherein the percent lymphocytes is <10%, there is a reduced suitability for manufacturing. In embodiments, wherein the percent lymphocytes is <40%, there is a reduced suitability for manufacturing. In embodiments, wherein the percent neutrophils is >60%, there is a reduced suitability for manufacturing. In embodiments, wherein the percent CD3+CD45+ cells (e.g., determined by flow cytometry) is <25%, there is a reduced suitability for manufacturing. In embodiments, wherein the percent monocytes is >60%, there is a reduced suitability for manufacturing.

In embodiments of any of the manufacturing or evaluating aspects herein, a sample with high suitability for manufacturing has an at least 50%, 60%, 70%, 80%, or 90% chance of manufacturing success. In embodiments, a sample with low suitability for manufacturing has less than 50%, 40%, 30%, 20%, or 10% chance of manufacturing success. In embodiment, evaluating the likelihood of manufacturing fail comprises identifying the sample as having at least a 50%, 60%, 70%, 80%, or 90% chance of undergoing manufacturing fail. In embodiment, evaluating the likelihood of manufacturing success comprises identifying the sample as having at least a 50%, 60%, 70%, 80%, or 90% chance of undergoing manufacturing success.

In embodiments of any of the manufacturing or evaluating aspects herein, e.g., embodiments where the sample has a high suitability for manufacturing, the method further comprises manufacturing one or more CAR-expressing cells from a sample from the subject. In one embodiment, the sample is the same sample that was assayed, and in another embodiment, the sample is a different sample from the subject. In embodiments, the method further comprises contacting a cell sample from the subject with a nucleic acid encoding CAR molecule, e.g., a CAR molecule described herein, e.g., a CD19 CAR. In embodiments the method further comprises freezing and thawing the apheresis sample. In embodiments, the method further comprises determining manufacturing fail or manufacturing success, e.g., based on cell expansion, CAR expression, or transduction efficiency. In embodiments, the method further comprises administering the manufactured cells to the subject.

In embodiments of any of the manufacturing or evaluating aspects herein, (e.g., embodiments where the sample has a low suitability for manufacturing), the method further comprises performing a second apheresis collection from the subject. In embodiments (e.g., embodiments where the sample has a low suitability for manufacturing) the method further comprises performing an enrichment, e.g., a modified enrichment, on the apheresis sample, e.g., the first or second apheresis sample. In embodiments the method further comprises freezing and thawing the apheresis sample, e.g., the first or second apheresis sample. In embodiments, the method further comprises evaluating T cell enrichment and/or decrease in suppressive non-lymphoid cells, e.g., myeloid derived suppressor cells (MDSC), e.g., after the second apheresis collection, e.g., after the enrichment or freezing and thawing, of the sample. In embodiments, a decrease in the level, e.g., percent or number, of CD45 dim or CD45 negative cells, e.g., relative to a reference sample (e.g., the first apheresis collection) is indicative of high suitability for manufacturing. In other embodiments, a decrease in the level, e.g., percent or number, of CD15-positive and/or CXCR2-positive cells, e.g., relative to a reference sample (e.g., the first apheresis collection) is indicative of high suitability for manufacturing.

In embodiments, (e.g., embodiments where the sample has a low suitability for manufacturing) the method further comprises discarding the cells in the assayed sample. In embodiments, the method further comprises manufacturing one or more CAR-expressing cells from the second apheresis sample. In embodiments, the first apheresis sample underwent manufacturing fail and the second apheresis sample underwent manufacturing success. In embodiments (e.g., embodiments where the sample has a low suitability for manufacturing), the method further comprises manufacturing one or more CAR-expressing cells from a sample from the subject. In one embodiment, the sample is the same sample that was assayed, and in another embodiment, the sample is a different sample from the subject.

In embodiments of any of the manufacturing or evaluating aspects herein, the method comprises performing or determining one or more of: complete blood count, flow cytometry phenotyping, cell size, and processing pathway on an apheresis sample.

In embodiments of any of the manufacturing or evaluating aspects herein, the method can further include performing a small scale test expansion (TE) to evaluate manufacturing proliferative capacity, e.g., one or more of cell number, cell phenotype (e.g., a cell phenotype as described herein), or transduction efficiency. In embodiments wherein the absolute lymphocyte count is <500/ul, the small scale test expansion can be used to evaluate suitability for manufacturing, e.g., high or low suitability for manufacturing. Small scale test expansion can be carried out, e.g., using the experimental conditions described in Example 37. For example, an aliquot of the apheresis sample can be obtained and cultured under small scale conditions similar to large scale manufacturing conditions.

In embodiments, a complete blood count with differential is a complete blood count that identifies the numbers or percentages of different types of blood cells, e.g., white blood cells, e.g., neutrophils, lymphocytes, monocytes, eosinophils, or basophils, in a sample.

In another aspect, the invention features a method of evaluating or monitoring the suitability of a sample (e.g., an apheresis sample or a manufactured CAR-expressing cell sample) for a CAR therapy (e.g., a CD19 CAR therapy). The method includes acquiring a value of sample suitability, wherein said value is indicative of the suitability of the CAR-expressing cell sample. In embodiments, the value of sample suitability, comprises a measure of the level or activity of a Stat3 signalling mediator (e.g., IL-6, IL-17, IL-22, IL-31, or CCL20 level or activity) in the CAR-expressing cell, wherein said value is indicative of a subject's responsiveness or relapsing status to the CAR-expressing cell, thereby evaluating the sample suitability.

In another aspect, the invention features a method of evaluating the suitability of a sample (e.g., an apheresis sample) for a CAR therapy (e.g., a CD19 CAR therapy). The method includes acquiring a value of sample suitability, wherein said value is indicative of the suitability of the CAR-expressing cell sample. In embodiments, the value of the sample suitability, comprises a measure of:
a) Ki-67 and/or granzyme B level, and
b) optionally, CD8 level,
c) optionally, CD45RO level, and/or
d) optionally, CD27 level,
wherein a Ki-67 level that is lower than a reference (e.g., lower than that in a CD8+CD45RO+CD27+ cell or population of cells) is indicative that a subject will be a CR or PR_{TD} to the CAR-expressing cell, and/or
wherein a granzyme B level that is higher than a reference (e.g., lower than that in a CD8+CD45RO+CD27+ cell or population of cells) is indicative that a subject will be a CR or PR_{TD} to the CAR-expressing cell.

In another aspect, the invention features a method of evaluating the suitability of a sample (e.g., an apheresis sample or a manufactured CAR-expressing cell sample) for a CAR therapy (e.g., a CD19 CAR therapy). The method includes acquiring a value of sample suitability, wherein said value is indicative of the suitability of the CAR-expressing cell therapy.

In embodiments, the value of sample suitability, comprises a measure of the level or activity of:
(i) CAR,
(ii) CD8, and
(iii) CD27, and/or PD1,
(e.g., CAR+CD8+CD27+PD1-) immune effector cells, e.g., in a T cell population, in a sample (e.g., an apheresis sample or a manufactured CAR-expressing cell product sample).

In some aspects, the present disclosure provides a method of evaluating a subject, e.g., evaluating or monitoring the effectiveness of a CAR-expressing cell therapy (e.g., CD19 CAR, (e.g., CTL019 or CTL119)) in a subject, having a cancer, comprising determining the persistence of the CAR-expressing cell in the subject (e.g., using qPCR or flow cytometry), wherein a persistence that is greater than a reference value (e.g., the average persistence in a NR or PD population) indicates a response, e.g., a complete response.

In embodiments, persistence is calculated by an area under the curve (AUC), e.g., AUC28 or AUC84. In embodiments (e.g., involving ALL), an AUC of above about 5×10⁵ or 1×10⁶ indicates CR. In embodiments (e.g., involving CLL), an AUC of above about 5×10⁵ or 1×10⁶ indicates CR or PR, and/or an AUC of below about 1×10⁵ or 5×10⁴ indicates NR/PD.

In embodiments, persistence is measured in the peripheral blood or bone marrow.

In embodiments, the AUC is determined at a preselected time period after administration of the CAR-expressing cell therapy. In some embodiments, the AUC is determined, e.g., between day 0 and day 45, between day 10 and day 40, between day 15 and day 35, between day 20 and day 30, or between day 0 and ending at day 25, 26, 27, 28, 29, or 30, after administration of the CAR-expressing cell therapy. In some embodiments, the AUC is determined, e.g., between day 0 and day 90, between, or between day 0 and ending at day 80, 82, 84, 85, 86, after administration of the CAR-expressing cell therapy.

In some embodiments, the methods disclosed herein further include administering a T cell depleting agent after treatment with the cell (e.g., an immune effector cell as described herein, e.g., an immune effector cell expressing CAR driven by a truncated PGK1 promoter), thereby reducing (e.g., depleting) the CAR-expressing cells (e.g., the CD19CAR-expressing cells). Such T cell depleting agents can be used to effectively deplete CAR-expressing cells (e.g., CD19CAR-expressing cells) to mitigate toxicity. In some embodiments, the CAR-expressing cells were manufactured according to a method herein, e.g., assayed (e.g., before or after transfection or transduction) according to a method herein.

In some embodiments, the T cell depleting agent is administered one, two, three, four, or five weeks after administration of the cell, e.g., the population of immune effector cells, described herein.

In one embodiment, the T cell depleting agent is an agent that depletes CAR-expressing cells, e.g., by inducing antibody dependent cell-mediated cytotoxicity (ADCC) and/or complement-induced cell death. For example, CAR-expressing cells described herein may also express an antigen (e.g., a target antigen) that is recognized by molecules capable of inducing cell death, e.g., ADCC or complement-induced cell death. For example, CAR expressing cells described herein may also express a target protein (e.g., a receptor) capable of being targeted by an antibody or antibody fragment. Examples of such target proteins include, but are not limited to, EpCAM, VEGFR, integrins (e.g., integrins αvβ3, α4, αI3/4β3, α4β7, α5β1, αvβ3, αv), members of the TNF receptor superfamily (e.g., TRAIL-R1, TRAIL-R2), PDGF Receptor, interferon receptor, folate receptor, GPNMB, ICAM-1, HLA-DR, CEA, CA-125, MUC1, TAG-72, IL-6 receptor, 5T4, GD2, GD3, CD2, CD3, CD4, CD5, CD11, CD11a/LFA-1, CD15, CD18/ITGB2, CD19, CD20, CD22, CD23/1gE Receptor, CD25, CD28, CD30, CD33, CD38, CD40, CD41, CD44, CD51, CD52, CD62L, CD74, CD80, CD125, CD147/basigin, CD152/CTLA-4, CD154/CD40L, CD195/CCR5, CD319/SLAMF7, and EGFR, and truncated versions thereof (e.g., versions preserving one or more extracellular epitopes but lacking one or more regions within the cytoplasmic domain).

In some embodiments, the CAR expressing cell co-expresses the CAR and the target protein, e.g., naturally expresses the target protein or is engineered to express the target protein. For example, the cell, e.g., the population of immune effector cells, can include a nucleic acid (e.g., vector) comprising the CAR nucleic acid (e.g., a CAR nucleic acid as described herein) and a nucleic acid encoding the target protein.

In one embodiment, the T cell depleting agent is a CD52 inhibitor, e.g., an anti-CD52 antibody molecule, e.g., alemtuzumab.

In other embodiments, the cell, e.g., the population of immune effector cells, expresses a CAR molecule as described herein (e.g., CD19CAR) and the target protein recognized by the T cell depleting agent. In one embodiment, the target protein is CD20. In embodiments where the target protein is CD20, the T cell depleting agent is an anti-CD20 antibody, e.g., rituximab.

In further embodiments of any of the aforesaid methods, the methods further include transplanting a cell, e.g., a hematopoietic stem cell, or a bone marrow, into the mammal.

In another aspect, the invention features a method of conditioning a mammal prior to cell transplantation. The method includes administering to the mammal an effective amount of the cell comprising a CAR nucleic acid or polypeptide, e.g., a CD19 CAR nucleic acid or polypeptide. In some embodiments, the cell transplantation is a stem cell transplantation, e.g., a hematopoietic stem cell transplantation, or a bone marrow transplantation. In other embodiments, conditioning a subject prior to cell transplantation includes reducing the number of target-expressing cells in a subject, e.g., CD19-expressing normal cells or CD19-expressing cancer cells.

### Biopolymer Delivery Methods

In some embodiments, one or more CAR-expressing cells as disclosed herein can be administered or delivered to the subject via a biopolymer scaffold, e.g., a biopolymer implant. Biopolymer scaffolds can support or enhance the delivery, expansion, and/or dispersion of the CAR-expressing cells described herein. A biopolymer scaffold comprises a biocompatible (e.g., does not substantially induce an inflammatory or immune response) and/or a biodegradable polymer that can be naturally occurring or synthetic. Exemplary biopolymers are described, e.g., in paragraphs 1004-1006 of International Application WO2015/142675, filed Mar. 13, 2015, which is herein incorporated by reference in its entirety.

### Therapeutic Applications

CD19 Associated Diseases and/or Disorders

In one aspect, the invention provides methods for treating a disease associated with CD19 expression. In one aspect, the invention provides methods for treating a disease wherein part of the cancer is negative for CD19 and part of the cancer is positive for CD19. For example, the methods and compositions of the invention are useful for treating subjects that have undergone treatment for a disease associated with expression of CD19, wherein the subject that has undergone treatment related to CD19 expression, e.g., treatment with a CD19 CAR, exhibits a disease associated with expression of CD19.

In one aspect, the invention pertains to a vector comprising CD19 CAR operably linked to promoter for expression in mammalian cells, e.g., T cells or NK cells. In one aspect, the invention provides a recombinant cell, e.g., a T cell or NK cell, expressing the CD19 CAR for use in treating CD19-expressing cancers, wherein the recombinant T cell expressing the CD19 CAR is termed a CD19 CART. In one aspect, the CD19 CART described herein, is capable of contacting a cancer cell with at least one CD19 CAR expressed on its surface such that the CART targets the cancer cell and growth of the cancer is inhibited.

In one aspect, the invention pertains to a method of inhibiting growth of a CD19-expressing cancer cell, comprising contacting the cancer cell with a CD19 CAR expressing cell, e.g., a CD19 CART cell, described, and one or more other CAR expressing cells, e.g., as described herein, such that the CART is activated in response to the antigen and targets the cancer cell, wherein the growth of the cancer is inhibited. The CD19 CAR-expressing cell, e.g., T cell, is administered in combination with a B-cell inhibitor, e.g., a B-cell inhibitor described herein.

The invention includes (among other things) a type of cellular therapy where T cells are genetically modified to express a chimeric antigen receptor (CAR) and the CAR T cell is infused to a recipient in need thereof. The infused cell is able to kill tumor cells in the recipient. Unlike antibody therapies, CAR-modified T cells are able to replicate in vivo resulting in long-term persistence that can lead to sustained tumor control. In various aspects, the T cells administered to the patient, or their progeny, persist in the patient for at least four months, five months, six months, seven months, eight months, nine months, ten months, eleven months, twelve months, thirteen months, fourteen month, fifteen months, sixteen months, seventeen months, eighteen months, nineteen months, twenty months, twenty-one months, twenty-two months, twenty-three months, two years, three years, four years, or five years after administration of the T cell to the patient.

The invention also includes a type of cellular therapy where immune effector cells, e.g., NK cells or T cells are modified, e.g., by in vitro transcribed RNA, to transiently express a chimeric antigen receptor (CAR) and the CAR-expressing (e.g., CAR T) cell is infused to a recipient in need thereof. The infused cell is able to kill cancer cells in the recipient. Thus, in various aspects, the CAR-expressing cells, e.g., T cells, administered to the patient, is present for less than one month, e.g., three weeks, two weeks, one week, after administration of the CAR-expressing cell, e.g., T cell, to the patient.

Without wishing to be bound by any particular theory, the anti-cancer immunity response elicited by the CAR-modified T cells may be an active or a passive immune response, or alternatively may be due to a direct vs indirect immune response. In one aspect, the CAR (e.g., CD19-CAR) transduced T cells exhibit specific proinflammatory cytokine secretion and potent cytolytic activity in response to human cancer cells expressing the target antigen (e.g., CD19), resist soluble target antigen inhibition, mediate bystander killing and mediate regression of an established human cancer. For example, antigen-less cancer cells within a heterogeneous field of target antigen-expressing cancer may be susceptible to indirect destruction by target antigen-redirected T cells that has previously reacted against adjacent antigen-positive cancer cells.

In one aspect, the CAR-modified cells of the invention, e.g., fully human CAR T cells, may be a type of vaccine for ex vivo immunization and/or in vivo therapy in a mammal. In one aspect, the mammal is a human.

With respect to ex vivo immunization, at least one of the following occurs in vitro prior to administering the cell into a mammal: i) expansion of the cells, ii) introducing a nucleic acid encoding a CAR to the cells or iii) cryopreservation of the cells.

Ex vivo procedures are well known in the art and are discussed more fully below. Briefly, cells are isolated from a mammal (e.g., a human) and genetically modified (i.e., transduced or transfected in vitro) with a vector expressing a CAR disclosed herein. The CAR-modified cell can be administered to a mammalian recipient to provide a therapeutic benefit. The mammalian recipient may be a human and the CAR-modified cell can be autologous with respect to the recipient. Alternatively, the cells can be allogeneic, syngeneic or xenogeneic with respect to the recipient.

The procedure for ex vivo expansion of hematopoietic stem and progenitor cells is described in U.S. Pat. No. 5,199,942, incorporated herein by reference, can be applied to the cells of the present invention. Other suitable methods are known in the art, therefore the present invention is not limited to any particular method of ex vivo expansion of the cells. Briefly, ex vivo culture and expansion of T cells can comprise: (1) collecting CD34+ hematopoietic stem and progenitor cells from a mammal from peripheral blood harvest or bone marrow explants; and (2) expanding such cells ex vivo. In addition to the cellular growth factors described in U.S. Pat. No. 5,199,942, other factors such as flt3-L, IL-1, IL-3 and c-kit ligand, can be used for culturing and expansion of the cells.

In addition to using a cell-based vaccine in terms of ex vivo immunization, also included in the methods described herein are compositions and methods for in vivo immunization to elicit an immune response directed against an antigen in a patient.

Generally, the cells activated and expanded as described herein may be utilized in the treatment and prevention of diseases that arise in individuals who are immunocompromised. In particular, the CAR-expressing cells described herein are used in the treatment of diseases, disorders and conditions associated with expression of one or more B-cell antigen. In certain aspects, the cells are used in the treatment of patients at risk for developing diseases, disorders and conditions associated with expression of one or more B-cell antigen. Thus, the present invention provides (among other things) methods for the treatment or prevention of diseases, disorders and conditions associated with expression of a B-cell antigen comprising administering to a subject in need thereof, a therapeutically effective amount of the CD19 CAR-expressing cells described herein, in combination with one or more of B-cell inhibitor described herein.

In one embodiment, the therapy described herein (e.g., a CD19 CAR therapy, and the cells expressing a CD19 CAR molecule, e.g., a CD19 CAR molecule described herein) are administered as a first line treatment for the disease, e.g., the cancer, e.g., the cancer described herein. In another embodiment, the therapy described herein (e.g., a CD19 CAR therapy, and the cells expressing a CD19 CAR molecule, e.g., a CD19 CAR molecule described herein) are administered as a second, third, fourth line treatment for the disease, e.g., the cancer, e.g., the cancer described herein.

The present invention also provides methods for inhibiting the proliferation or reducing a CD19-expressing cell population, the methods comprising contacting a population of cells comprising a CD19-expressing cell with an anti-CD19 CAR-expressing cell described herein that binds to the CD19-expressing cell, and contacting the population of CD19-expressing cells with one or more of a B-cell inhibitor described herein. In a specific aspect, the present invention provides methods for inhibiting the proliferation or reducing the population of cancer cells expressing CD19, the methods comprising contacting the CD19-expressing cancer cell population with an anti-CD19 CAR-expressing cell described herein that binds to the CD19-expressing cell, and contacting the CD19-expressing cell with one or more B-cell described herein. In one aspect, the present invention provides methods for inhibiting the proliferation or reducing the population of cancer cells expressing CD19, the methods comprising contacting the CD19-expressing cancer cell population with an anti-CD19 CAR-expressing cell described herein that binds to the CD19-expressing cell and contacting the CD19-expressing cell with one or more B-cell described herein. In certain aspects, the combination of the anti-CD19 CAR-expressing cell described herein and one or more B-cell described herein reduces the quantity, number, amount or percentage of cells and/or cancer cells by at least 25%, at least 30%, at least 40%, at least 50%, at least 65%, at least 75%, at least 85%, at least 95%, or at least 99% in a subject with or animal model for a hematological cancer or another cancer associated with CD19-expressing cells relative to a negative control. In one aspect, the subject is a human.

The present invention also provides methods for preventing, treating and/or managing a disease associated with CD19-expressing cells (e.g., a hematologic cancer or atypical cancer expressing CD19), the methods comprising administering to a subject in need an anti-CD19 CAR-expressing cell that binds to the CD19-expressing cell. In one aspect, the subject is a human. Non-limiting examples of disorders associated with CD19-expressing cells include autoimmune disorders (such as lupus), inflammatory disorders (such as allergies and asthma) and cancers (such as hematological cancers or atypical cancers expressing CD19).

The present invention also provides methods for preventing, treating and/or managing a disease associated with CD19-expressing cells, the methods comprising administering to a subject in need an anti-CD19 CART cell of the invention that binds to the CD19-expressing cell. In one aspect, the subject is a human.

The present invention also provides methods for preventing relapse of cancer associated with CD19-expressing cells, the methods comprising administering to a subject in need thereof an anti-CD19 CART cell of the invention that binds to the CD19-expressing cell. In one aspect, the methods comprise administering to the subject in need thereof an effective amount of an anti-CD19 CART cell described herein that binds to the CD19-expressing cell in combination with an effective amount of another therapy.

In one aspect, the invention pertains to a method of treating cancer in a subject. The method comprises administering to the subject a CD19 CAR-expressing cell, e.g., T cell, described herein, such that the cancer is treated in the subject. An example of a cancer that is treatable by the methods described herein is a cancer associated with expression of CD19. In one embodiment, the disease is a solid or liquid tumor. In one embodiment, the disease is a hematologic cancer, e.g., as described herein.

Non-cancer related indications associated with expression of CD19 include, but are not limited to, e.g., autoimmune disease, (e.g., lupus), inflammatory disorders (allergy and asthma) and transplantation.

In one aspect, the CAR of the invention can be used to eradicate CD19-expressing normal cells, thereby applicable for use as a cellular conditioning therapy prior to cell transplantation. In one aspect, the CD19-expressing normal cell is a CD19-expressing normal stem cell and the cell transplantation is a stem cell transplantation, e.g., as described herein.

In some embodiments, a cancer that can be treated with the combination described herein is multiple myeloma. Multiple myeloma is a cancer of the blood, characterized by accumulation of a plasma cell clone in the bone marrow. Current therapies for multiple myeloma include, but are not limited to, treatment with lenalidomide, which is an analog of thalidomide. Lenalidomide has activities which include anti-tumor activity, angiogenesis inhibition, and immunomodulation. In some embodiments, a CD19 CAR, e.g., as described herein, may be used to target myeloma cells. In some embodiments, the combination described herein can be used with one or more additional therapies, e.g., lenalidomide treatment.

The CAR-expressing cells described herein may be administered either alone, or as a pharmaceutical composition in combination with diluents and/or with other components such as IL-2, IL-15, IL-7, IL-21 or other cytokines or cell populations.

### Hematologic Cancers

Hematological cancer conditions are the types of cancer such as leukemia, lymphoma and malignant lymphoproliferative conditions that affect blood, bone marrow and the lymphatic system.

In one embodiment, the hematologic cancer is leukemia. In one embodiment, the cancer is selected from the group consisting of one or more acute leukemias including but not limited to B-cell acute lymphoid leukemia (BALL), T-cell acute lymphoid leukemia (TALL), small lymphocytic leukemia (SLL), acute lymphoid leukemia (ALL); one or more chronic leukemias including but not limited to chronic myelogenous leukemia (CML), chronic lymphocytic leukemia (CLL); additional hematologic cancers or hematologic conditions including, but not limited to mantle cell lymphoma (MCL), B cell prolymphocytic leukemia, blastic plasmacytoid dendritic cell neoplasm, Burkitt's lymphoma, diffuse large B cell lymphoma, follicular lymphoma, hairy cell leukemia, small cell- or a large cell-follicular lymphoma, malignant lymphoproliferative conditions, MALT lymphoma, Marginal zone lymphoma, multiple myeloma, myelodysplasia and myelodysplastic syndrome, non-Hodgkin lymphoma, Hodgkin lymphoma, plasmablastic lymphoma, plasmacytoid dendritic cell neoplasm, Waldenstrom macroglobulinemia, and “preleukemia” which are a diverse collection of hematological conditions united by ineffective production (or dysplasia) of myeloid blood cells. Diseases associated with CD19, CD20, or CD22 expression include, but not limited to atypical and/or non-classical cancers, malignancies, precancerous conditions or proliferative diseases expressing CD19, CD20, or CD22; and any combination thereof.

Leukemia can be classified as acute leukemia and chronic leukemia. Acute leukemia can be further classified as acute myelogenous leukemia (AML) and acute lymphoid leukemia (ALL). Chronic leukemia includes chronic myelogenous leukemia (CML) and chronic lymphoid leukemia (CLL). Other related conditions include myelodysplastic syndromes (MDS, formerly known as “preleukemia”) which are a diverse collection of hematological conditions united by ineffective production (or dysplasia) of myeloid blood cells and risk of transformation to AML.

Lymphoma is a group of blood cell tumors that develop from lymphocytes. Exemplary lymphomas include non-Hodgkin lymphoma and Hodgkin lymphoma.

In an aspect, the invention pertains to a method of treating a mammal having Hodgkin lymphoma, comprising administering to the mammal an effective amount of the cells expressing a CD19 CAR molecule, e.g., a CD19 CAR molecule described herein and a B-cell inhibitor.

In one aspect, the compositions and CART cells or CAR expressing NK cells of the present invention are particularly useful for treating B cell malignancies, such as non-Hodgkin lymphomas, e.g., DLBCL, Follicular lymphoma, or CLL.

Non-Hodgkin lymphoma (NHL) is a group of cancers of lymphocytes, formed from either B or T cells. NHLs occur at any age and are often characterized by lymph nodes that are larger than normal, weight loss, and fever. Different types of NHLs are categorized as aggressive (fast-growing) and indolent (slow-growing) types. B-cell non-Hodgkin lymphomas include Burkitt lymphoma, chronic lymphocytic leukemia/small lymphocytic lymphoma (CLL/SLL), diffuse large B-cell lymphoma (DLBCL), follicular lymphoma, immunoblastic large cell lymphoma, precursor B-lymphoblastic lymphoma, and mantle cell lymphoma. Examples of T-cell non-Hodgkin lymphomas include mycosis fungoides, anaplastic large cell lymphoma, and precursor T-lymphoblastic lymphoma. Lymphomas that occur after bone marrow or stem cell transplantation are typically B-cell non-Hodgkin lymphomas. See, e.g., Maloney. NEJM. 366.21(2012):2008-16.

Diffuse large B-cell lymphoma (DLBCL) is a form of NHL that develops from B cells. DLBCL is an aggressive lymphoma that can arise in lymph nodes or outside of the lymphatic system, e.g., in the gastrointestinal tract, testes, thyroid, skin, breast, bone, or brain. Three variants of cellular morphology are commonly observed in DLBCL: centroblastic, immunoblastic, and anaplastic. Centroblastic morphology is most common and has the appearance of medium-to-large-sized lymphocytes with minimal cytoplasm. There are several subtypes of DLBCL. For example, primary central nervous system lymphoma is a type of DLBCL that only affects the brain is called and is treated differently than DLBCL that affects areas outside of the brain. Another type of DLBCL is primary mediastinal B-cell lymphoma, which often occurs in younger patients and grows rapidly in the chest. Symptoms of DLBCL include a painless rapid swelling in the neck, armpit, or groin, which is caused by enlarged lymph nodes. For some subjects, the swelling may be painful. Other symptoms of DLBCL include night sweats, unexplained fevers, and weight loss. Although most patients with DLBCL are adults, this disease sometimes occurs in children. Treatment for DLBCL includes chemotherapy (e.g., cyclophosphamide, doxorubicin, vincristine, prednisone, etoposide), antibodies (e.g., Rituxan), radiation, or stem cell transplants.

Follicular lymphoma a type of non-Hodgkin lymphoma and is a lymphoma of follicle center B-cells (centrocytes and centroblasts), which has at least a partially follicular pattern. Follicular lymphoma cells express the B-cell markers CD10, CD19, CD20, and CD22. Follicular lymphoma cells are commonly negative for CD5. Morphologically, a follicular lymphoma tumor is made up of follicles containing a mixture of centrocytes (also called cleaved follicle center cells or small cells) and centroblasts (also called large noncleaved follicle center cells or large cells). The follicles are surrounded by non-malignant cells, mostly T-cells. The follicles contain predominantly centrocytes with a minority of centroblasts. The World Health Organization (WHO) morphologically grades the disease as follows: grade 1 (<5 centroblasts per high-power field (hpf); grade 2 (6-15 centroblasts/hpf); grade 3 (>15 centroblasts/hpf). Grade 3 is further subdivided into the following grades: grade 3A (centrocytes still present); grade 3B (the follicles consist almost entirely of centroblasts). Treatment of follicular lymphoma includes chemotherapy, e.g., alkyating agents, nucleoside analogs, anthracycline-containing regimens, e.g., a combination therapy called CHOP—cyclophosphamide, doxorubicin, vincristine, prednisone/prednisolone, antibodies (e.g., rituximab), radioimmunotherapy, and hematopoietic stem cell transplantation.

CLL is a B-cell malignancy characterized by neoplastic cell proliferation and accumulation in bone morrow, blood, lymph nodes, and the spleen. The median age at time of diagnosis of CLL is about 65 years. Current treatments include chemotherapy, radiation therapy, biological therapy, or bone marrow transplantation. Sometimes symptoms are treated surgically (e.g., splenectomy removal of enlarged spleen) or by radiation therapy (e.g., de-bulking swollen lymph nodes). Chemotherapeutic agents to treat CLL include, e.g., fludarabine, 2-chlorodeoxyadenosine (cladribine), chlorambucil, vincristine, pentostatin, cyclophosphamide, alemtuzumab (Campath-1H), doxorubicin, and prednisone. Biological therapy for CLL includes antibodies, e.g., alemtuzumab, rituximab, and ofatumumab; as well as tyrosine kinase inhibitor therapies. A number of criteria can be used to classify stage of CLL, e.g., the Rai or Binet system. The Rai system describes CLL has having five stages: stage 0 where only lymphocytosis is present; stage I where lymphadenopathy is present; stage II where splenomegaly, lymphadenopathy, or both are present; stage III where anemia, organomegaly, or both are present (progression is defined by weight loss, fatigue, fever, massive organomegaly, and a rapidly increasing lymphocyte count); and stage IV where anemia, thrombocytopenia, organomegaly, or a combination thereof are present. Under the Binet staging system, there are three categories: stage A where lymphocytosis is present and less than three lymph nodes are enlarged (this stage is inclusive of all Rai stage 0 patients, one-half of Rai stage I patients, and one-third of Rai stage II patients); stage B where three or more lymph nodes are involved; and stage C wherein anemia or thrombocytopenia, or both are present. These classification systems can be combined with measurements of mutation of the immunoglobulin genes to provide a more accurate characterization of the state of the disease. The presence of mutated immunoglobulin genes correlates to improved prognosis.

In another embodiment, the CAR expressing cells of the present invention are used to treat cancers or leukemias, e.g., with leukemia stem cells. For example, the leukemia stem cells are CD34⁺/CD38⁻ leukemia cells.

In some embodiments, a CAR-expressing cell (e.g., CD19 CAR-expressing cell) described herein is used to deplete a B cell (e.g., a population of B cells, e.g., regulatory B cells). Without wishing to be bound by theory, it is believed that depletion of B cells, e.g., regulatory B cells, can improve the tumor microenvironment such that anti-cancer therapies (e.g., therapies described herein) can be more effective (e.g., than without depletion of the B cells). Thus, provided herein is a method for reducing, e.g., depleting, regulatory cells (e.g., regulatory B cells). The method includes administering a CAR-expressing cell (e.g., CD19 CAR-expressing cell) described herein in an amount sufficient to reduce the regulatory cells. In some embodiments, the methods can be used to modulate a tumor microenvironment, e.g., to enhance the effectiveness of a therapy described herein.

In some embodiments, a dose of CAR-expressing cells (e.g., CAR-expressing cells described herein, e.g., CD19 CAR-expressing cells described herein) comprises about 10⁴ to about 10⁹ cells/kg, e.g., about 10⁴ to about 10⁵ cells/kg, about 10⁵ to about 10⁶ cells/kg, about 10⁶ to about 10⁷ cells/kg, about 10⁷ to about 10⁸ cells/kg, or about 10⁸ to about 10⁹ cells/kg. In embodiments, the dose of CAR-expressing cells comprises about 0.6×10⁶ cells/kg to about 2×10⁷ cells/kg.

In some embodiments, a dose of CAR-expressing cells described herein (e.g., CD19 CAR-expressing cells) comprises about 2×10⁵, 1×10⁶, 1.1×10⁶, 2×10⁶, 3×10⁶, 3.6×10⁶, 5×10⁶, 1×10⁷, 1.8×10⁷, 2×10⁷, 5×10⁷, 1×10⁸, 2×10⁸, 3×10⁸, or 5×10⁸ cells/kg. In some embodiments, a dose of CAR cells (e.g., CD19 CAR-expressing cells) comprises at least about 1×10⁶, 1.1×10⁶, 2×10⁶, 3.6×10⁶, 5×10⁶, 1×10⁷, 1.8×10⁷, 2×10⁷, 5×10⁷, 1×10⁸, 2×10⁸, 3×10⁸, or 5×10⁸ cells/kg. In some embodiments, a dose of CAR cells (e.g., CD19 CAR-expressing cells) comprises up to about 1×10⁶, 1.1×10⁶, 2×10⁶, 3.6×10⁶, 5×10⁶, 1×10⁷, 1.8×10⁷, 2×10⁷, 5×10⁷, 1×10⁸, 2×10⁸, 3×10⁸, or 5×10⁸ cells/kg. In some embodiments, a dose of CAR cells (e.g., CD19 CAR-expressing cells) comprises about 1.1×10⁶-1.8×10⁷ cells/kg. In some embodiments, a dose of CAR cells (e.g., CD19 CAR-expressing cell) comprises about 1×10⁷, 2×10⁷, 5×10⁷, 1×10⁸, 2×10⁸, 3×10⁸, 5×10⁸, 1×10⁹, 2×10⁹, or 5×10⁹ cells. In some embodiments, a dose of CAR cells (e.g., e.g., CD19 CAR-expressing cells) comprises at least about 1×10⁷, 2×10⁷, 5×10⁷, 1×10⁸, 2×10⁸, 3×10⁸, 5×10⁸, 1×10⁹, 2×10⁹, or 5×10⁹ cells. In some embodiments, a dose of CAR cells (e.g., e.g., CD19 CAR-expressing cells) comprises up to about 1×10⁷, 2×10⁷, 5×10⁷, 1×10⁸, 2×10⁸, 3×10⁸, 5×10⁸, 1×10⁹, 2×10⁹, or 5×10⁹ cells.

In some embodiments, a dose of CAR cells (e.g., CD19 CAR-expressing cells) comprises up to about 1×10⁷, 1.5×10⁷, 2×10⁷, 2.5×10⁷, 3×10⁷, 3.5×10⁷, 4×10⁷, 5×10⁷, 1×10⁸, 1.5×10⁸, 2×10⁸, 2.5×10⁸, 3×10⁸, 3.5×10⁸, 4×10⁸, 5×10⁸, 1×10⁹, 2×10⁹, or 5×10⁹ cells. In some embodiments, a dose of CAR cells (e.g., CD19 CAR-expressing cell) comprises up to about 1-3×10⁷ to 1-3×10⁸ cells. In some embodiments, the subject is administered about 1-3×10⁷ of CD19 CAR-expressing cells. In other embodiments, the subject is administered about 1-3×10⁸ of CD19 CAR-expressing cells.

In some embodiments, a dose of CAR-expressing cells (e.g., CAR-expressing cells described herein, e.g., CD19 CAR-expressing cells described herein) comprises about 1×10⁶ cells/m² to about 1×10⁹ cells/m², e.g., about 1×10⁷ cells/m² to about 5×10⁸ cells/m², e.g., about 1.5×10⁷ cells/m², about 2×10⁷ cells/m², about 4.5×10⁷ cells/m², about 10⁸ cells/m², about 1.2×10⁸ cells/m², or about 2×10⁸ cells/m².

### Selected Doses and Dosage Regimens

Accordingly, in one aspect, the invention pertains to a method of treating a subject (e.g., a mammal) having a cancer, comprising administering immune effector cells comprising a CAR molecule. In one embodiment, the immune effector cells are administered as a single dose, e.g., a single dose as described herein. In other embodiments the immune effector cells are administered as a plurality of doses, e.g., a first dose, a second dose, and optionally a third dose, e.g., as described herein.

In a related aspect, the invention pertains to a method of treating a subject (e.g., an adult subject) having a cancer (e.g., acute lymphoid leukemia (ALL)), comprising administering to the subject a dose, e.g., a single dose, or a plurality of doses (e.g., a first dose, a second dose, and optionally one or more additional doses), each dose comprising immune effector cells expressing a CAR molecule, e.g., a CD19 CAR molecule, (e.g., a CAR molecule according to residues 22-486 of SEQ ID NO: 58) or a BCMA CAR molecule.

In yet another aspect, the invention pertains to a use of a single dose, or a plurality of doses (e.g., a first dose, a second dose, and optionally a third dose), of immune effector cells comprising a CAR molecule (e.g., a CD19 CAR molecule, (e.g., a CAR molecule according to residues 22-486 of SEQ ID NO: 58) or a BCMA CAR molecule), for treating a subject (e.g., an adult) having a cancer (e.g., acute lymphoid leukemia (ALL)).

In certain aspects, the invention features a method of treating a subject (e.g., a pediatric subject) having a cancer (e.g., ALL), comprising administering to the subject immune effector cells expressing a CAR molecule. The method comprises administering one of the following:
(i) administering a dose of 2-5×10⁶ viable CAR-expressing cells/kg, e.g., transduced viable T cells cells/kg, wherein the subject has a body mass of less than or equal to 50 kg;
(ii) administering a dose of 1.0-2.5×10⁸ viable CAR-expressing cells, e.g., transduced viable T cells, wherein the subject has a body mass of at least 50 kg;
(iii) administering a dose of 0.2-5×10⁶ viable CAR-expressing cells/kg, e.g., transduced viable T cells/kg, wherein the subject has a body mass of less than or equal to 50 kg; or
(iv) administering a dose of 0.1-2.5×10⁸ viable CAR-expressing cells, e.g., transduced viable T cells, wherein the subject has a body mass of at least 50 kg.

In embodiments, a single dose is administered to the subject, e.g., the pediatric subject. In embodiments, the CAR-expressing cell is a CD19 CAR-expressing cell, e.g., a cell expressing a CD19 CAR of any Tables 2 or 3 herein, e.g., CTL019 or CTL119.

In embodiments, the doses are administered on sequential days, e.g., the first dose is administered on day 1, the second dose is administered on day 2, and the optional third dose (if administered) is administered on day 3.

In embodiments, a fourth, fifth, or sixth dose, or more doses, are administered.

In embodiments, the first dose comprises about 10% of the total dose, the second dose comprises about 30% of the total dose, and the third dose comprises about 60% of the total dose, wherein the aforementioned percentages have a sum of 100%. In embodiments, the first dose comprises about 9-11%, 8-12%, 7-13%, or 5-15% of the total dose. In embodiments, the second dose comprises about 29-31%, 28-32%, 27-33%, 26-34%, 25-35%, 24-36%, 23-37%, 22-38%, 21-39%, or 20-40% of the total dose. In embodiments, the third dose comprises about 55-65%, 50-70%, 45-75%, or 40-80% of the total dose. In embodiments, the total dose refers to the total number of viable CAR-expressing cells administered over the course of 1 week, 2 weeks, 3 weeks, or 4 weeks. In some embodiments wherein two doses are administered, the total dose refers to the sum of the number of viable CAR-expressing cells administered to the subject in the first and second doses. In some embodiments wherein three doses are administered, the total dose refers to the sum of the number of viable CAR-expressing cells administered to the subject in the first, second, and third doses.

In embodiments, the dose is measured according to the number of viable CAR-expressing cells therein. CAR expression can be measured, e.g., by flow cytometry using an antibody molecule that binds the CAR molecule and a detectable label. Viability can be measured, e.g., by Cellometer.

In embodiments, the viable CAR-expressing cells are administered in ascending doses. In embodiments, the second dose is larger than the first dose, e.g., larger by 10%, 20%, 30%, or 50%. In embodiments, the second dose is twice, three times, four times, or five times the size of the first dose. In embodiments, the third dose is larger than the second dose, e.g., larger by 10%, 20%, 30%, or 50%. In embodiments, the third dose is twice, three times, four times, or five times the size of the second dose.

In certain embodiments, the method includes one, two, three, four, five, six, seven or all of a)-h) of the following:
a) the number of CAR-expressing, viable cells administered in the first dose is no more than ⅓, of the number of CAR-expressing, viable cells administered in the second dose;
b) the number of CAR-expressing, viable cells administered in the first dose is no more than 1/X, wherein X is 2, 3, 4, 5, 6, 7, 8, 9, 10, 15, 20, 30, 40 or 50, of the total number of CAR-expressing, viable cells administered;
c) the number of CAR-expressing, viable cells administered in the first dose is no more than 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, or 5×10⁸ CAR-expressing, viable cells, and the second dose is greater than the first dose;
d) the number of CAR-expressing, viable cells administered in the second dose is no more than ½, of the number of CAR-expressing, viable cells administered in the third dose;
e) the number of CAR-expressing, viable cells administered in the second dose is no more than 1/Y, wherein Y is 2, 3, 4, 5, 6, 7, 8, 9, 10, 15, 20, 30, 40 or 50, of the total number of CAR-expressing, viable cells administered;
f) the number of CAR-expressing, viable cells administered in the second dose is no more than 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, or 5×10⁸ CAR-expressing, viable cells, and the third dose is greater than the second dose;
h) the dosages and time periods of administration of the first, second, and optionally third doses are selected such that the subject experiences CRS at a level no greater than 4, 3, 2, or 1.

In embodiments, the total dose is about 5×10⁸ CAR-expressing, viable cells. In embodiments, the total dose is about 5×10⁷-5×10⁸ CAR-expressing, viable cells. In embodiments, the first dose is about 5×10⁷ (e.g., ±10%, 20%, or 30%) CAR-expressing, viable cells, the second dose is about 1.5×10⁸ (e.g., ±10%, 20%, or 30%) CAR-expressing, viable cells, and the third dose is about 3×10⁸ (e.g., ±10%, 20%, or 30%) CAR-expressing, viable cells.

In embodiments, the method comprises administering, e.g., as a single dose or as a plurality of doses as described herein, a dose of 0.02-5×10⁶ viable CAR-expressing cells/kg, e.g., transduced viable T cells/kg, e.g., a dose of 0.02-5×10⁶, 0.03-5×10⁶, 0.04-5×10⁶, 0.05-5×10⁶, 0.06-5×10⁶, 0.07-5×10⁶, 0.08-5×10⁶, 0.09-5×10⁶, 0.10-5×10⁶, 0.11-5×10⁶, 0.12-5×10⁶, 0.13-5×10⁶, 0.14-5×10⁶, 0.15-5×10⁶, 0.16-5×10⁶, 0.17-5×10⁶, 0.18-5×10⁶, 0.19-5×10⁶, or 0.20-5×10⁶, wherein the subject has a body mass of less than or equal to 50 kg.

In embodiments, the method comprises administering, e.g., as a single dose or as a plurality of doses as described herein, a dose of 0.2-5×10⁶ viable CAR-expressing cells/kg, e.g., transduced viable T cells/kg, e.g., a dose of 0.2-5×10⁶, 0.3-5×10⁶, 0.4-5×10⁶, 0.5-5×10⁶, 0.6-5×10⁶, 0.7-5×10⁶, 0.8-5×10⁶, 0.9-5×10⁶, 1.0-5×10⁶, 1.1-5×10⁶, 1.2-5×10⁶, 1.3-5×10⁶, 1.4-5×10⁶, 1.5-5×10⁶, 1.6-5×10⁶, 1.7-5×10⁶, 1.8-5×10⁶, 1.9-5×10⁶, or 2-5×10⁶, wherein the subject has a body mass of less than or equal to 50 kg.

In embodiments, the method comprises administering, e.g., as a single dose or as a plurality of doses as described herein, a dose of 2-50×10⁶ viable CAR-expressing cells/kg, e.g., transduced viable T cells/kg, e.g., a dose of 2-5×10⁶, 2-10×10⁶, 2-15×10⁶, 2-20×10⁶, 2-25×10⁶, 2-30×10⁶, 2-35×10⁶, 2-40×10⁶, 2-45×10⁶, or 2-50×10⁶, wherein the subject has a body mass of less than or equal to 50 kg.

In embodiments, the method further comprises administering, e.g., as a single dose or as a plurality of doses as described herein, a dose of 0.2-50×10⁶ viable CAR-expressing cells/kg, e.g., transduced viable T cells/kg, e.g., a dose of 0.2-5×10⁶, 0.2-10×10⁶, 0.2-15×10⁶, 0.2-20×10⁶, 0.2-25×10⁶, 0.2-30×10⁶, 0.2-35×10⁶, 0.2-40×10⁶, 0.2-45×10⁶, or 0.2-50×10⁶, wherein the subject has a body mass of less than or equal to 50 kg.

In embodiments, the method comprises administering, e.g., as a single dose or as a plurality of doses as described herein, a dose of 0.02-50×10⁶ viable CAR-expressing cells/kg, e.g., transduced viable T cells/kg, e.g, a dose of 0.02-50×10⁶, 0.03-45×10⁶, 0.05-40×10⁶, 0.1-35×10⁶, 0.2-30×10⁶, 0.3-25×10⁶, 0.4-20×10⁶, 0.5-15×10⁶, 1-10×10⁶, 2-5×10⁶, wherein the subject has a body mass of less than or equal to 50 kg.

In embodiments, the method comprises administering, e.g., as a single dose or as a plurality of doses as described herein, a dose of 0.01-2.5×10⁸ viable CAR-expressing cells, e.g., transduced viable T cells, e.g., a dose of 0.01-2.5×10⁸, 0.02-2.5×10⁸, 0.03-2.5×10⁸, 0.04-2.5×10⁸, 0.05-2.5×10⁸, 0.06-2.5×10⁸, 0.07-2.5×10⁸, 0.08-2.5×10⁸, 0.09-2.5×10⁸, or 0.10-2.5×10⁸, wherein the subject has a body mass of at least 50 kg.

In embodiments, the method comprises administering, e.g., as a single dose or as a plurality of doses as described herein, a dose of 0.1-2.5×10⁸ viable CAR-expressing cells, e.g., transduced viable T cells, e.g., a dose of 0.1-2.5×10⁸, 0.2-2.5×10⁸, 0.3-2.5×10⁸, 0.4-2.5×10⁸, 0.5-2.5×10⁸, 0.6-2.5×10⁸, 0.7-2.5×10⁸, 0.8-2.5×10⁸, 0.9-2.5×10⁸, or 1.0-2.5×10⁸, wherein the subject has a body mass of at least 50 kg.

In embodiments, the method comprises administering, e.g., as a single dose or as a plurality of doses as described herein, a dose of 1-25×10⁸ viable CAR-expressing cells, e.g., transduced viable T cells/kg, e.g., a dose of 1-2.5×10⁸, 1-5×10⁸, 1-7.5×10⁸, 1-10×10⁸, 1-12.5×10⁸, 1-15×10⁸, 1-17.5×10⁸, 1-20×10⁸, 1-22.5×10⁸, or 1-25×10⁸, wherein the subject has a body mass of at least 50 kg.

In embodiments, the method comprises administering, e.g., as a single dose or as a plurality of doses as described herein, a dose of 0.1-2.5×10⁸ viable CAR-expressing cells, e.g., transduced viable T cells/kg, e.g., a dose of 0.1-2.5×10⁸, 0.1-5×10⁸, 0.1-7.5×10⁸, 0.1-10×10⁸, 0.1-12.5×10⁸, 0.1-15×10⁸, 0.1-17.5×10⁸, 0.1-20×10⁸, 0.1-22.5×10⁸, or 0.1-25×10⁸, wherein the subject has a body mass of at least 50 kg.

In embodiments, the method comprises administering, e.g., as a single dose or as a plurality of doses as described herein, a dose of 0.01-25×10⁸ viable CAR-expressing cells, e.g., transduced viable T cells/kg, e.g., a dose of 0.01-25×10⁸, 0.05-22.5×10⁸, 0.1-20×10⁸, 0.2-17.5×10⁸, 0.5-15×10⁸, 0.6-12.5×10⁸, 0.7-10×10⁸, 0.8-7.5×10⁸, 0.9-5×10⁸, or 1-2.5×10⁸, wherein the subject has a body mass of at least 50 kg.

In any of the methods or compositions for use described herein, in some embodiments, a dose of CAR-expressing cells (e.g., CD19 CAR-expressing cells) comprises about 10⁴ to about 10⁹ cells/kg, e.g., about 10⁴ to about 10⁵ cells/kg, about 10⁵ to about 10⁶ cells/kg, about 10⁶ to about 10⁷ cells/kg, about 10⁷ to about 10⁸ cells/kg, or about 10⁸ to about 10⁹ cells/kg; or at least about one of: 1×10⁷, 1.5×10⁷, 2×10⁷, 2.5×10⁷, 3×10⁷, 3.5×10⁷, 4×10⁷, 5×10⁷, 1×10⁸, 1.5×10⁸, 2×10⁸, 2.5×10⁸, 3×10⁸, 3.5×10⁸, 4×10⁸, 5×10⁸, 1×10⁹, 2×10⁹, or 5×10⁹ cells. In some embodiments, a dose of CAR-expressing cells (e.g., CD19 CAR-expressing cells or BCMA CAR-expressing cells) comprises at least about 1-5×10⁷ to 1-5×10⁸ CAR-expressing cells. In some embodiments, the subject is administered about 1-5×10⁷ CAR-expressing cells (e.g., CD19 CAR-expressing cells or BCMA CAR-expressing cells)). In other embodiments, the subject is administered about 1-5×10⁸ CAR-expressing cells (e.g., CD19 CAR-expressing cells or BCMA CAR-expressing cells)).

Any of the dose ranges disclosed herein is intended to include the upper and lower endpoint values specified. For example, a dose range of 1-5×10⁷ CAR-expressing cells includes a dose of 1×10⁷ CAR-expressing cells and 5×10⁷ CAR-expressing cells (unless explicitly noted otherwise).

### Combination Therapies

The combination of a CAR as described herein (e.g., a CD19 CAR-expressing cell described herein) may be used in combination with other known agents and therapies.

A CAR-expressing cell described herein and/or the at least one additional therapeutic agent can be administered simultaneously, in the same or in separate compositions, or sequentially. For sequential administration, the CAR-expressing cell described herein can be administered first, and the additional agent can be administered second, or the order of administration can be reversed.

The CAR therapy and/or other therapeutic agents (such as a second CAR therapy), procedures or modalities can be administered during periods of active disorder, or during a period of remission or less active disease. The CAR therapy can be administered before the other treatment, concurrently with the treatment, post-treatment, or during remission of the disorder.

For instance, in some embodiments, CAR therapy is administered to a subject having a disease associated with CD19 expression, e.g., a cancer. The subject can be assayed for indicators of responsiveness or relapse. In some embodiments, when the subject shows one or more signs of relapse, e.g., a frameshift and/or premature stop codon in CD19, an additional therapy is administered. In embodiments, the additional therapy is a B-cell inhibitor. The CD19 therapy may be continued (for instance, when there are still some CD19-expressing cancer cells detectable in the subject) or may be discontinued (for instance, when a risk-benefit analysis favors discontinuing the therapy).

When administered in combination, the CAR therapy and the additional agent (e.g., second or third agent), or all, can be administered in an amount or dose that is higher, lower or the same than the amount or dosage of each agent used individually, e.g., as a monotherapy. In certain embodiments, the administered amount or dosage of the CAR therapy, the additional agent (e.g., second or third agent), or all, is lower (e.g., at least 20%, at least 30%, at least 40%, or at least 50%) than the amount or dosage of each agent used individually, e.g., as a monotherapy. In other embodiments, the amount or dosage of the CAR therapy, the additional agent (e.g., second or third agent), or all, that results in a desired effect (e.g., treatment of cancer) is lower (e.g., at least 20%, at least 30%, at least 40%, or at least 50% lower) than the amount or dosage of each agent used individually, e.g., as a monotherapy, required to achieve the same therapeutic effect.

The one or more therapies described herein can be administered to the subject substantially at the same time or in any order. For instance, a CD19 inhibitor, e.g., a CD19 CAR-expressing cell described herein, and/or optionally the at least one additional therapeutic agent can be administered simultaneously, in the same or in separate compositions, or sequentially. Additional timings of administration, e.g., sequence of administration, are described in pages 4-15 of International Application WO 2016/164731, filed Apr. 8, 2016, which is incorporated by reference in its entirety

In embodiments, one or more of the therapeutics in the combination therapy is an antibody molecule. Cancer antigens can be targeted with monoclonal antibody therapy. Monoclonal antibody (mAb) therapy has been shown to exert powerful antitumor effects by multiple mechanisms, including complement-dependent cytotoxicity (CDC), antibody-dependent cellular cytotoxicity (ADCC) and direct cell inhibition or apoptosis-inducing effects on tumor cells that over-express the target molecules.

In further aspects, the combination of the CAR-expressing cell described herein may be used in a treatment regimen in combination with surgery, chemotherapy, radiation, an mTOR pathway inhibitor, immunosuppressive agents, such as cyclosporin, azathioprine, methotrexate, mycophenolate, and FK506, antibodies, or other immunoablative agents such as CAMPATH, anti-CD3 antibodies or other antibody therapies, cytoxin, fludarabine, cyclosporin, FK506, rapamycin, mycophenolic acid, steroids, FR901228, cytokines, and irradiation. peptide vaccine, such as that described in Izumoto et al. 2008 J Neurosurg 108:963-971.

In one embodiment, the CAR-expressing cell described herein (optionally in combination with a B-cell inhibitor) can be used in combination with a chemotherapeutic agent. Exemplary chemotherapeutic agents include an anthracycline (e.g., doxorubicin (e.g., liposomal doxorubicin)); a vinca alkaloid (e.g., vinblastine, vincristine, vindesine, vinorelbine); an alkylating agent (e.g., cyclophosphamide, decarbazine, melphalan, ifosfamide, temozolomide); an immune cell antibody (e.g., alemtuzamab, gemtuzumab, rituximab, tositumomab); an antimetabolite (including, e.g., folic acid antagonists, pyrimidine analogs, purine analogs and adenosine deaminase inhibitors (e.g., fludarabine)); a TNFR glucocorticoid induced TNFR related protein (GITR) agonist; a proteasome inhibitor (e.g., aclacinomycin A, gliotoxin or bortezomib); an immunomodulator such as thalidomide or a thalidomide derivative (e.g., lenalidomide).

General Chemotherapeutic agents considered for use in combination therapies include anastrozole (Arimidex®), bicalutamide (Casodex®), bleomycin sulfate (Blenoxane®), busulfan (Myleran®), busulfan injection (Busulfex®), capecitabine (Xeloda®), N4-pentoxycarbonyl-5-deoxy-5-fluorocytidine, carboplatin (Paraplatin®), carmustine (BiCNU®), chlorambucil (Leukeran®), cisplatin (Platinol®), cladribine (Leustatin®), cyclophosphamide (Cytoxan® or Neosar®), cytarabine, cytosine arabinoside (Cytosar-U®), cytarabine liposome injection (DepoCyt®), dacarbazine (DTIC-Dome®), dactinomycin (Actinomycin D, Cosmegan), daunorubicin hydrochloride (Cerubidine®), daunorubicin citrate liposome injection (DaunoXome®), dexamethasone, docetaxel (Taxotere®), doxorubicin hydrochloride (Adriamycin®, Rubex®), etoposide (Vepesid®), fludarabine phosphate (Fludara®), 5-fluorouracil (Adrucil®, Efudex®), flutamide (Eulexin®), tezacitibine, gemcitabine (difluorodeoxycitidine), hydroxyurea (Hydrea®), Idarubicin (Idamycin®), ifosfamide (IFEX®), irinotecan (Camptosar®), L-asparaginase (ELSPAR®), leucovorin calcium, melphalan (Alkeran®), 6-mercaptopurine (Purinethol®), methotrexate (Folex®), mitoxantrone (Novantrone®), mylotarg, paclitaxel (Taxol®), nab-paclitaxel (Abraxane®), phoenix (Yttrium90/MX-DTPA), pentostatin, polifeprosan 20 with carmustine implant (Gliadel®), tamoxifen citrate (Nolvadex®), teniposide (Vumon®), 6-thioguanine, thiotepa, tirapazamine (Tirazone®), topotecan hydrochloride for injection (Hycamptin®), vinblastine (Velban®), vincristine (Oncovin®), and vinorelbine (Navelbine®).

Treatment with a combination of a chemotherapeutic agent and a cell expressing a CAR molecule described herein can be used to treat a hematologic cancer described herein, e.g., AML. In embodiments, the combination of a chemotherapeutic agent and a CAR-expressing cell is useful for targeting, e.g., killing, cancer stem cells, e.g., leukemic stem cells, e.g., in subjects with AML. In embodiments, the combination of a chemotherapeutic agent and a CAR-expressing cell is useful for treating minimal residual disease (MRD). MRD refers to the small number of cancer cells that remain in a subject during treatment, e.g., chemotherapy, or after treatment. MRD is often a major cause for relapse. The present invention provides a method for treating cancer, e.g., MRD, comprising administering a chemotherapeutic agent in combination with a CAR-expressing cell, e.g., as described herein.

In an embodiment, the chemotherapeutic agent is administered prior to administration of the cell expressing a CAR molecule, e.g., a CAR molecule described herein. In chemotherapeutic regimens where more than one administration of the chemotherapeutic agent is desired, the chemotherapeutic regimen is initiated or completed prior to administration of a cell expressing a CAR molecule, e.g., a CAR molecule described herein. In embodiments, the chemotherapeutic agent is administered at least 1 day, 2 days, 3 days, 4 days, 5 days, 6 days, 7 days, 8 days, 9 days, 10 days, 11 days, 12 days, 13 days, 14 days, 15 days, 20 days, 25 days, or 30 days prior to administration of the cell expressing the CAR molecule. In embodiments, the chemotherapeutic regimen is initiated or completed at least 1 day, 2 days, 3 days, 4 days, 5 days, 6 days, 7 days, 8 days, 9 days, 10 days, 11 days, 12 days, 13 days, 14 days, 15 days, 20 days, 25 days, or 30 days prior to administration of the cell expressing the CAR molecule.

Anti-cancer agents of particular interest for combinations with the compounds of the present invention include: antimetabolites; drugs that inhibit either the calcium dependent phosphatase calcineurin or the p70S6 kinase FK506) or inhibit the p70S6 kinase; alkylating agents; mTOR inhibitors; immunomodulators; anthracyclines; vinca alkaloids; proteosome inhibitors; GITR agonists; protein tyrosine phosphatase inhibitors; a CDK4 kinase inhibitor; a BTK kinase inhibitor; a MKN kinase inhibitor; a DGK kinase inhibitor; or an oncolytic virus.

Exemplary antimetabolites include, without limitation, folic acid antagonists (also referred to herein as antifolates), pyrimidine analogs, purine analogs and adenosine deaminase inhibitors): methotrexate (Rheumatrex®, Trexall®), 5-fluorouracil (Adrucil®, Efudex®, Fluoroplex®), floxuridine (FUDF®), cytarabine (Cytosar-U®, Tarabine PFS), 6-mercaptopurine (Puri-Nethol®)), 6-thioguanine (Thioguanine Tabloid®), fludarabine phosphate (Fludara®), pentostatin (Nipent®), pemetrexed (Alimta®), raltitrexed (Tomudex®), cladribine (Leustatin®), clofarabine (Clofarex®, Clolar®), mercaptopurine (Puri-Nethol®), capecitabine (Xeloda®), nelarabine (Arranon®), azacitidine (Vidaza®) and gemcitabine (Gemzar®). Preferred antimetabolites include, e.g., 5-fluorouracil (Adrucil®, Efudex®, Fluoroplex®), floxuridine (FUDF®), capecitabine (Xeloda®), pemetrexed (Alimta®), raltitrexed (Tomudex®) and gemcitabine (Gemzar®).

Exemplary alkylating agents include, without limitation, nitrogen mustards, ethylenimine derivatives, alkyl sulfonates, nitrosoureas and triazenes): uracil mustard (Aminouracil Mustard®, Chlorethaminacil®, Demethyldopan®, Desmethyldopan®, Haemanthamine®, Nordopan®, Uracil nitrogen Mustard®, Uracillost®, Uracilmostaza®, Uramustin®, Uramustine®), chlormethine (Mustargen®), cyclophosphamide (Cytoxan®, Neosar®, Clafen®, Endoxan®, Procytox®, Revimmune™), ifosfamide (Mitoxana®), melphalan (Alkeran®), Chlorambucil (Leukeran®), pipobroman (Amedel®, Vercyte®), triethylenemelamine (Hemel®, Hexalen®, Hexastat®), triethylenethiophosphoramine, Temozolomide (Temodar®), thiotepa (Thioplex®), busulfan (Busilvex®, Myleran®), carmustine (BiCNU®), lomustine (CeeNU®), streptozocin (Zanosar®), and Dacarbazine (DTIC-Dome®). Additional exemplary alkylating agents include, without limitation, Oxaliplatin (Eloxatin®); Temozolomide (Temodar® and Temodal®); Dactinomycin (also known as actinomycin-D, Cosmegen®); Melphalan (also known as L-PAM, L-sarcolysin, and phenylalanine mustard, Alkeran®); Altretamine (also known as hexamethylmelamine (HMM), Hexalen®); Carmustine (BiCNU®); Bendamustine (Treanda®); Busulfan (Busulfex® and Myleran®); Carboplatin (Paraplatin®); Lomustine (also known as CCNU, CeeNU®); Cisplatin (also known as CDDP, Platinol® and Platinol®-AQ); Chlorambucil (Leukeran®); Cyclophosphamide (Cytoxan® and Neosar®); Dacarbazine (also known as DTIC, DIC and imidazole carboxamide, DTIC-Dome®); Altretamine (also known as hexamethylmelamine (HMM), Hexalen®); Ifosfamide (Ifex®); Prednumustine; Procarbazine (Matulane®); Mechlorethamine (also known as nitrogen mustard, mustine and mechloroethamine hydrochloride, Mustargen®); Streptozocin (Zanosar®); Thiotepa (also known as thiophosphoamide, TESPA and TSPA, Thioplex®); Cyclophosphamide (Endoxan®, Cytoxan®, Neosar®, Procytox®, Revimmune®); and Bendamustine HCl (Treanda®).

In embodiments, a CAR-expressing cell described herein is administered to a subject in combination with fludarabine, cyclophosphamide, and/or rituximab. In embodiments, a CAR-expressing cell described herein is administered to a subject in combination with fludarabine, cyclophosphamide, and rituximab (FCR). In embodiments, the subject has CLL. For example, the subject has a deletion in the short arm of chromosome 17 (del(17p), e.g., in a leukemic cell). In other examples, the subject does not have a del(17p). In embodiments, the subject comprises a leukemic cell comprising a mutation in the immunoglobulin heavy-chain variable-region (IgV_{H}) gene. In other embodiments, the subject does not comprise a leukemic cell comprising a mutation in the immunoglobulin heavy-chain variable-region (IgV_{H}) gene. In embodiments, the fludarabine is administered at a dosage of about 10-50 mg/m² (e.g., about 10-15, 15-20, 20-25, 25-30, 30-35, 35-40, 40-45, or 45-50 mg/m²), e.g., intravenously. In embodiments, the cyclophosphamide is administered at a dosage of about 200-300 mg/m² (e.g., about 200-225, 225-250, 250-275, or 275-300 mg/m²), e.g., intravenously. In embodiments, the rituximab is administered at a dosage of about 400-600 mg/m2 (e.g., 400-450, 450-500, 500-550, or 550-600 mg/m²), e.g., intravenously.

In embodiments, a CAR-expressing cell described herein is administered to a subject in combination with bendamustine and rituximab. In embodiments, the subject has CLL. For example, the subject has a deletion in the short arm of chromosome 17 (del(17p), e.g., in a leukemic cell). In other examples, the subject does not have a del(17p). In embodiments, the subject comprises a leukemic cell comprising a mutation in the immunoglobulin heavy-chain variable-region (IgV_{H}) gene. In other embodiments, the subject does not comprise a leukemic cell comprising a mutation in the immunoglobulin heavy-chain variable-region (IgV_{H}) gene. In embodiments, the bendamustine is administered at a dosage of about 70-110 mg/m2 (e.g., 70-80, 80-90, 90-100, or 100-110 mg/m2), e.g., intravenously. In embodiments, the rituximab is administered at a dosage of about 400-600 mg/m2 (e.g., 400-450, 450-500, 500-550, or 550-600 mg/m²), e.g., intravenously.

In embodiments, a CAR-expressing cell described herein is administered to a subject in combination with rituximab, cyclophosphamide, doxorubicine, vincristine, and/or a corticosteroid (e.g., prednisone). In embodiments, a CAR-expressing cell described herein is administered to a subject in combination with rituximab, cyclophosphamide, doxorubicine, vincristine, and prednisone (R-CHOP). In embodiments, the subject has diffuse large B-cell lymphoma (DLBCL). In embodiments, the subject has nonbulky limited-stage DLBCL (e.g., comprises a tumor having a size/diameter of less than 7 cm). In embodiments, the subject is treated with radiation in combination with the R-CHOP. For example, the subject is administered R-CHOP (e.g., 1-6 cycles, e.g., 1, 2, 3, 4, 5, or 6 cycles of R-CHOP), followed by radiation. In some cases, the subject is administered R-CHOP (e.g., 1-6 cycles, e.g., 1, 2, 3, 4, 5, or 6 cycles of R-CHOP) following radiation.

In embodiments, a CAR-expressing cell described herein is administered to a subject in combination with etoposide, prednisone, vincristine, cyclophosphamide, doxorubicin, and/or rituximab. In embodiments, a CAR-expressing cell described herein is administered to a subject in combination with etoposide, prednisone, vincristine, cyclophosphamide, doxorubicin, and rituximab (EPOCH-R). In embodiments, a CAR-expressing cell described herein is administered to a subject in combination with dose-adjusted EPOCH-R (DA-EPOCH-R). In embodiments, the subject has a B cell lymphoma, e.g., a Myc-rearranged aggressive B cell lymphoma.

In embodiments, a CAR-expressing cell described herein is administered to a subject in combination with rituximab and/or lenalidomide. Lenalidomide ((RS)-3-(4-Amino-1-oxo 1,3-dihydro-2H-isoindol-2-yl)piperidine-2,6-dione) is an immunomodulator. In embodiments, a CAR-expressing cell described herein is administered to a subject in combination with rituximab and lenalidomide. In embodiments, the subject has follicular lymphoma (FL) or mantle cell lymphoma (MCL). In embodiments, the subject has FL and has not previously been treated with a cancer therapy. In embodiments, lenalidomide is administered at a dosage of about 10-20 mg (e.g., 10-15 or 15-20 mg), e.g., daily. In embodiments, rituximab is administered at a dosage of about 350-550 mg/m² (e.g., 350-375, 375-400, 400-425, 425-450, 450-475, or 475-500 mg/m²), e.g., intravenously.

Exemplary mTOR inhibitors include, e.g., temsirolimus; ridaforolimus (formally known as deferolimus, (1R,2R,4S)-4-[(2R)-2 [(1R,9S,12S,15R,16E,18R,19R,21R,23S,24E,26E,28Z,30S,32S,35R)-1,18-dihydroxy-19,30-dimethoxy-15,17,21,23,29,35-hexamethyl-2,3,10,14,20-pentaoxo-11,36-dioxa-4-azatricyclo[30.3.1.0^{4,9}] hexatriaconta-16,24,26,28-tetraen-12-yl]propyl]-2-methoxycyclohexyl dimethylphosphinate, also known as AP23573 and MK8669, and described in PCT Publication No. WO 03/064383); everolimus (Afinitor® or RAD001); rapamycin (AY22989, Sirolimus®); simapimod (CAS 164301-51-3); emsirolimus, (5-{2,4-Bis[(3S)-3-methylmorpholin-4-yl]pyrido[2,3-d]pyrimidin-7-yl}-2-methoxyphenyl)methanol (AZD8055); 2-Amino-8-[trans-4-(2-hydroxyethoxy)cyclohexyl]-6-(6-methoxy-3-pyridinyl)-4-methyl-pyrido[2,3-d]pyrimidin-7(8H)-one (PF04691502, CAS 1013101-36-4); and N²-[1,4-dioxo-4-[[4-(4-oxo-8-phenyl-4H-1-benzopyran-2-yl)morpholinium-4-yl]methoxy]butyl]-L-arginylglycyl-L-α-aspartylL-serine-, inner salt (SF1126, CAS 936487-67-1) (SEQ ID NO: 1316), and XL765.

Exemplary immunomodulators include, e.g., afutuzumab (available from Roche®); pegfilgrastim (Neulasta®); lenalidomide (CC-5013, Revlimid®); thalidomide (Thalomid®), actimid (CC4047); and IRX-2 (mixture of human cytokines including interleukin 1, interleukin 2, and interferon γ, CAS 951209-71-5, available from IRX Therapeutics).

Exemplary anthracyclines include, e.g., doxorubicin (Adriamycin® and Rubex®); bleomycin (Lenoxane®); daunorubicin (dauorubicin hydrochloride, daunomycin, and rubidomycin hydrochloride, Cerubidine®); daunorubicin liposomal (daunorubicin citrate liposome, DaunoXome®); mitoxantrone (DHAD, Novantrone®); epirubicin (Ellence™); idarubicin (Idamycin®, Idamycin PFS®); mitomycin C (Mutamycin®); geldanamycin; herbimycin; ravidomycin; and desacetylravidomycin.

Exemplary vinca alkaloids include, e.g., vinorelbine tartrate (Navelbine®), Vincristine (Oncovin®), and Vindesine (Eldisine®)); vinblastine (also known as vinblastine sulfate, vincaleukoblastine and VLB, Alkaban-AQ® and Velban®); and vinorelbine (Navelbine®).

Exemplary proteosome inhibitors include bortezomib (Velcade®); carfilzomib (PX-171-007, (S)-4-Methyl-N—((S)-1-(((S)-4-methyl-1-((R)-2-methyloxiran-2-yl)-1-oxopentan-2-yl)amino)-1-oxo-3-phenylpropan-2-yl)-2-((S)-2-(2-morpholinoacetamido)-4-phenylbutanamido)-pentanamide); marizomib (NPI-0052); ixazomib citrate (MLN-9708); delanzomib (CEP-18770); and O-Methyl-N-[(2-methyl-5-thiazolyl)carbonyl]-L-seryl-O-methyl-N-[(1S)-2-[(2R)-2-methyl-2-oxiranyl]-2-oxo-1-(phenylmethyl)ethyl]-L-serinamide (ONX-0912).

In embodiments, a CAR-expressing cell described herein is administered to a subject in combination with brentuximab. Brentuximab is an antibody-drug conjugate of anti-CD30 antibody and monomethyl auristatin E. In embodiments, the subject has Hodgkin's lymphoma (HL), e.g., relapsed or refractory HL. In embodiments, the subject comprises CD30+HL. In embodiments, the subject has undergone an autologous stem cell transplant (ASCT). In embodiments, the subject has not undergone an ASCT. In embodiments, brentuximab is administered at a dosage of about 1-3 mg/kg (e.g., about 1-1.5, 1.5-2, 2-2.5, or 2.5-3 mg/kg), e.g., intravenously, e.g., every 3 weeks.

In embodiments, a CAR-expressing cell described herein is administered to a subject in combination with brentuximab and dacarbazine or in combination with brentuximab and bendamustine. Dacarbazine is an alkylating agent with a chemical name of 5-(3,3-Dimethyl-1-triazenyl)imidazole-4-carboxamide. Bendamustine is an alkylating agent with a chemical name of 4-[5-[Bis(2-chloroethyl)amino]-1-methylbenzimidazol-2-yl]butanoic acid. In embodiments, the subject has Hodgkin's lymphoma (HL). In embodiments, the subject has not previously been treated with a cancer therapy. In embodiments, the subject is at least 60 years of age, e.g., 60, 65, 70, 75, 80, 85, or older. In embodiments, dacarbazine is administered at a dosage of about 300-450 mg/m² (e.g., about 300-325, 325-350, 350-375, 375-400, 400-425, or 425-450 mg/m²), e.g., intravenously. In embodiments, bendamustine is administered at a dosage of about 75-125 mg/m2 (e.g., 75-100 or 100-125 mg/m², e.g., about 90 mg/m²), e.g., intravenously. In embodiments, brentuximab is administered at a dosage of about 1-3 mg/kg (e.g., about 1-1.5, 1.5-2, 2-2.5, or 2.5-3 mg/kg), e.g., intravenously, e.g., every 3 weeks.

In some embodiments, a CAR-expressing cell described herein is administered to a subject in combination with a CD20 inhibitor, e.g., an anti-CD20 antibody (e.g., an anti-CD20 mono- or bispecific antibody) or a fragment thereof. Exemplary anti-CD20 antibodies include but are not limited to rituximab, ofatumumab, ocrelizumab, veltuzumab, obinutuzumab, TRU-015 (Trubion Pharmaceuticals), ocaratuzumab, and Pro131921 (Genentech). See, e.g., Lim et al. Haematologica. 95.1 (2010): 135-43.

In some embodiments, the anti-CD20 antibody comprises rituximab. Rituximab is a chimeric mouse/human monoclonal antibody IgG1 kappa that binds to CD20 and causes cytolysis of a CD20 expressing cell, e.g., as described in www.accessdata.fda.gov/drugsatfda_docs/label/2010/103705s5311lbl.pdf. In embodiments, a CAR-expressing cell described herein is administered to a subject in combination with rituximab. In embodiments, the subject has CLL or SLL.

In some embodiments, rituximab is administered intravenously, e.g., as an intravenous infusion. For example, each infusion provides about 500-2000 mg (e.g., about 500-550, 550-600, 600-650, 650-700, 700-750, 750-800, 800-850, 850-900, 900-950, 950-1000, 1000-1100, 1100-1200, 1200-1300, 1300-1400, 1400-1500, 1500-1600, 1600-1700, 1700-1800, 1800-1900, or 1900-2000 mg) of rituximab. In some embodiments, rituximab is administered at a dose of 150 mg/m² to 750 mg/m², e.g., about 150-175 mg/m², 175-200 mg/m², 200-225 mg/m², 225-250 mg/m², 250-300 mg/m², 300-325 mg/m², 325-350 mg/m², 350-375 mg/m², 375-400 mg/m², 400-425 mg/m², 425-450 mg/m², 450-475 mg/m², 475-500 mg/m², 500-525 mg/m², 525-550 mg/m², 550-575 mg/m², 575-600 mg/m², 600-625 mg/m², 625-650 mg/m², 650-675 mg/m², or 675-700 mg/m², where m² indicates the body surface area of the subject. In some embodiments, rituximab is administered at a dosing interval of at least 4 days, e.g., 4, 7, 14, 21, 28, 35 days, or more. For example, rituximab is administered at a dosing interval of at least 0.5 weeks, e.g., 0.5, 1, 2, 3, 4, 5, 6, 7, 8 weeks, or more. In some embodiments, rituximab is administered at a dose and dosing interval described herein for a period of time, e.g., at least 2 weeks, e.g., at least 2, 3, 4, 5, 6, 7, 8, 9, 10, 11, 12, 13, 14, 15, 16, 17, 18, 19, 20 weeks, or greater. For example, rituximab is administered at a dose and dosing interval described herein for a total of at least 4 doses per treatment cycle (e.g., at least 4, 5, 6, 7, 8, 9, 10, 11, 12, 13, 14, 15, 16, or more doses per treatment cycle).

In some embodiments, the anti-CD20 antibody comprises ofatumumab. Ofatumumab is an anti-CD20 IgG1κ human monoclonal antibody with a molecular weight of approximately 149 kDa. For example, ofatumumab is generated using transgenic mouse and hybridoma technology and is expressed and purified from a recombinant murine cell line (NS0). See, e.g., www.accessdata.fda.gov/drugsatfda_docs/label/2009/125326lbl.pdf; and Clinical Trial Identifier number NCT01363128, NCT01515176, NCT01626352, and NCT01397591. In embodiments, a CAR-expressing cell described herein is administered to a subject in combination with ofatumumab. In embodiments, the subject has CLL or SLL.

In some embodiments, ofatumumab is administered as an intravenous infusion. For example, each infusion provides about 150-3000 mg (e.g., about 150-200, 200-250, 250-300, 300-350, 350-400, 400-450, 450-500, 500-550, 550-600, 600-650, 650-700, 700-750, 750-800, 800-850, 850-900, 900-950, 950-1000, 1000-1200, 1200-1400, 1400-1600, 1600-1800, 1800-2000, 2000-2200, 2200-2400, 2400-2600, 2600-2800, or 2800-3000 mg) of ofatumumab. In embodiments, ofatumumab is administered at a starting dosage of about 300 mg, followed by 2000 mg, e.g., for about 11 doses, e.g., for 24 weeks. In some embodiments, ofatumumab is administered at a dosing interval of at least 4 days, e.g., 4, 7, 14, 21, 28, 35 days, or more. For example, ofatumumab is administered at a dosing interval of at least 1 week, e.g., 1, 2, 3, 4, 5, 6, 7, 8, 9, 10, 11, 12, 24, 26, 28, 20, 22, 24, 26, 28, 30 weeks, or more. In some embodiments, ofatumumab is administered at a dose and dosing interval described herein for a period of time, e.g., at least 1 week, e.g., 1, 2, 3, 4, 5, 6, 7, 8, 9, 10, 11, 12, 13, 14, 15, 16, 17, 18, 19, 20, 22, 24, 26, 28, 30, 40, 50, 60 weeks or greater, or 1, 2, 3, 4, 5, 6, 7, 8, 9, 10, 11, 12 months or greater, or 1, 2, 3, 4, 5 years or greater. For example, ofatumumab is administered at a dose and dosing interval described herein for a total of at least 2 doses per treatment cycle (e.g., at least 2, 3, 4, 5, 6, 7, 8, 9, 10, 11, 12, 13, 14, 15, 16, 18, 20, or more doses per treatment cycle).

In some cases, the anti-CD20 antibody comprises ocrelizumab. Ocrelizumab is a humanized anti-CD20 monoclonal antibody, e.g., as described in Clinical Trials Identifier Nos. NCT00077870, NCT01412333, NCT00779220, NCT00673920, NCT01194570, and Kappos et al. Lancet. 19.378(2011):1779-87.

In some cases, the anti-CD20 antibody comprises veltuzumab. Veltuzumab is a humanized monoclonal antibody against CD20. See, e.g., Clinical Trial Identifier No. NCT00547066, NCT00546793, NCT01101581, and Goldenberg et al. Leuk Lymphoma. 51(5)(2010):747-55.

In some cases, the anti-CD20 antibody comprises GA101. GA101 (also called obinutuzumab or R05072759) is a humanized and glyco-engineered anti-CD20 monoclonal antibody. See, e.g., Robak. Curr. Opin. Investig. Drugs. 10.6(2009):588-96; Clinical Trial Identifier Numbers: NCT01995669, NCT01889797, NCT02229422, and NCT01414205; and www.accessdata.fda.gov/drugsatfda_docs/label/2013/125486s000lbl.pdf.

In some cases, the anti-CD20 antibody comprises AME-133v. AME-133v (also called LY2469298 or ocaratuzumab) is a humanized IgG1 monoclonal antibody against CD20 with increased affinity for the FcγRIIIa receptor and an enhanced antibody dependent cellular cytotoxicity (ADCC) activity compared with rituximab. See, e.g., Robak et al. BioDrugs 25.1(2011):13-25; and Forero-Torres et al. Clin Cancer Res. 18.5(2012):1395-403.

In some cases, the anti-CD20 antibody comprises PRO131921. PRO131921 is a humanized anti-CD20 monoclonal antibody engineered to have better binding to FcγRIIIa and enhanced ADCC compared with rituximab. See, e.g., Robak et al. BioDrugs 25.1(2011):13-25; and Casulo et al. Clin Immunol. 154.1(2014):37-46; and Clinical Trial Identifier No. NCT00452127.

In some cases, the anti-CD20 antibody comprises TRU-015. TRU-015 is an anti-CD20 fusion protein derived from domains of an antibody against CD20. TRU-015 is smaller than monoclonal antibodies, but retains Fc-mediated effector functions. See, e.g., Robak et al. BioDrugs 25.1(2011):13-25. TRU-015 contains an anti-CD20 single-chain variable fragment (scFv) linked to human IgG1 hinge, CH2, and CH3 domains but lacks CH1 and CL domains.

In some embodiments, an anti-CD20 antibody described herein is conjugated or otherwise bound to a therapeutic agent, e.g., a chemotherapeutic agent (e.g., cytoxan, fludarabine, histone deacetylase inhibitor, demethylating agent, peptide vaccine, anti-tumor antibiotic, tyrosine kinase inhibitor, alkylating agent, anti-microtubule or anti-mitotic agent), anti-allergic agent, anti-nausea agent (or anti-emetic), pain reliever, or cytoprotective agent described herein.

In embodiments, a CAR-expressing cell described herein is administered to a subject in combination with a B-cell lymphoma 2 (BCL-2) inhibitor (e.g., venetoclax, also called ABT-199 or GDC-0199) and/or rituximab. In embodiments, a CAR-expressing cell described herein is administered to a subject in combination with venetoclax and rituximab. Venetoclax is a small molecule that inhibits the anti-apoptotic protein, BCL-2. Venetoclax has the following chemical name (4-(4-{[2-(4-chlorophenyl)-4,4-dimethylcyclohex-1-en-1-yl]methyl}piperazin-1-yl)-N-({3-nitro-4-[(tetrahydro-2H-pyran-4-ylmethyl)amino]phenyl}sulfonyl)-2-(1H-pyrrolo[2,3-b]pyridin-5-yloxy)benzamide).

In embodiments, the subject has CLL. In embodiments, the subject has relapsed CLL, e.g., the subject has previously been administered a cancer therapy. In embodiments, venetoclax is administered at a dosage of about 15-600 mg (e.g., 15-20, 20-50, 50-75, 75-100, 100-200, 200-300, 300-400, 400-500, or 500-600 mg), e.g., daily. In embodiments, rituximab is administered at a dosage of about 350-550 mg/m2 (e.g., 350-375, 375-400, 400-425, 425-450, 450-475, or 475-500 mg/m2), e.g., intravenously, e.g., monthly.

In some embodiments, one or more CAR-expressing cells described herein is administered in combination with an oncolytic virus. In embodiments, oncolytic viruses are capable of selectively replicating in and triggering the death of or slowing the growth of a cancer cell. In some cases, oncolytic viruses have no effect or a minimal effect on non-cancer cells. An oncolytic virus includes but is not limited to an oncolytic adenovirus, oncolytic Herpes Simplex Viruses, oncolytic retrovirus, oncolytic parvovirus, oncolytic vaccinia virus, oncolytic Sinbis virus, oncolytic influenza virus, or oncolytic RNA virus (e.g., oncolytic reovirus, oncolytic Newcastle Disease Virus (NDV), oncolytic measles virus, or oncolytic vesicular stomatitis virus (VSV)).

In some embodiments, the oncolytic virus is a virus, e.g., recombinant oncolytic virus, described in US2010/0178684 A1, which is incorporated herein by reference in its entirety. In some embodiments, a recombinant oncolytic virus comprises a nucleic acid sequence (e.g., heterologous nucleic acid sequence) encoding an inhibitor of an immune or inflammatory response, e.g., as described in US2010/0178684 A1, incorporated herein by reference in its entirety. In embodiments, the recombinant oncolytic virus, e.g., oncolytic NDV, comprises a pro-apoptotic protein (e.g., apoptin), a cytokine (e.g., GM-CSF, interferon-gamma, interleukin-2 (IL-2), tumor necrosis factor-alpha), an immunoglobulin (e.g., an antibody against ED-B firbonectin), tumor associated antigen, a bispecific adapter protein (e.g., bispecific antibody or antibody fragment directed against NDV HN protein and a T cell co-stimulatory receptor, such as CD3 or CD28; or fusion protein between human IL-2 and single chain antibody directed against NDV HN protein). See, e.g., Zamarin et al. Future Microbiol. 7.3(2012):347-67, incorporated herein by reference in its entirety. In some embodiments, the oncolytic virus is a chimeric oncolytic NDV described in U.S. Pat. No. 8,591,881 B2, US 2012/0122185 A1, or US 2014/0271677 A1, each of which is incorporated herein by reference in their entireties.

In some embodiments, the oncolytic virus comprises a conditionally replicative adenovirus (CRAd), which is designed to replicate exclusively in cancer cells. See, e.g., Alemany et al. Nature Biotechnol. 18(2000):723-27. In some embodiments, an oncolytic adenovirus comprises one described in Table 1 on page 725 of Alemany et al., incorporated herein by reference in its entirety.

Exemplary oncolytic viruses include but are not limited to the following:

Group B Oncolytic Adenovirus (ColoAd1) (PsiOxus Therapeutics Ltd.) (see, e.g., Clinical Trial Identifier: NCT02053220);

ONCOS-102 (previously called CGTG-102), which is an adenovirus comprising granulocyte-macrophage colony stimulating factor (GM-CSF) (Oncos Therapeutics) (see, e.g., Clinical Trial Identifier: NCT01598129);

VCN-01, which is a genetically modified oncolytic human adenovirus encoding human PH20 hyaluronidase (VCN Biosciences, S.L.) (see, e.g., Clinical Trial Identifiers: NCT02045602 and NCT02045589);

Conditionally Replicative Adenovirus ICOVIR-5, which is a virus derived from wild-type human adenovirus serotype 5 (Had5) that has been modified to selectively replicate in cancer cells with a deregulated retinoblastoma/E2F pathway (Institut Català d'Oncologia) (see, e.g., Clinical Trial Identifier: NCT01864759);

Celyvir, which comprises bone marrow-derived autologous mesenchymal stem cells (MSCs) infected with ICOVIR5, an oncolytic adenovirus (Hospital Infantil Universitario Niño Jesús, Madrid, Spain/Ramon Alemany) (see, e.g., Clinical Trial Identifier: NCT01844661);

CG0070, which is a conditionally replicating oncolytic serotype 5 adenovirus (Ad5) in which human E2F-1 promoter drives expression of the essential E1a viral genes, thereby restricting viral replication and cytotoxicity to Rb pathway-defective tumor cells (Cold Genesys, Inc.) (see, e.g., Clinical Trial Identifier: NCT02143804); or

DNX-2401 (formerly named Delta-24-RGD), which is an adenovirus that has been engineered to replicate selectively in retinoblastoma (Rb)-pathway deficient cells and to infect cells that express certain RGD-binding integrins more efficiently (Clinica Universidad de Navarra, Universidad de Navarra/DNAtrix, Inc.) (see, e.g., Clinical Trial Identifier: NCT01956734).

In some embodiments, an oncolytic virus described herein is administering by injection, e.g., subcutaneous, intra-arterial, intravenous, intramuscular, intrathecal, or intraperitoneal injection. In embodiments, an oncolytic virus described herein is administered intratumorally, transdermally, transmucosally, orally, intranasally, or via pulmonary administration.

In an embodiment, cells expressing a CAR described herein are administered to a subject in combination with a molecule that decreases the Treg cell population. Methods that decrease the number of (e.g., deplete) Treg cells are known in the art and include, e.g., CD25 depletion, cyclophosphamide administration, modulating GITR function. Without wishing to be bound by theory, it is believed that reducing the number of Treg cells in a subject prior to apheresis or prior to administration of a CAR-expressing cell described herein reduces the number of unwanted immune cells (e.g., Tregs) in the tumor microenvironment and reduces the subject's risk of relapse.

In an embodiment, a CAR-expressing cell described herein is administered to a subject in combination with a molecule that decreases the Treg cell population. Methods that decrease the number of (e.g., deplete) Treg cells are known in the art and include, e.g., CD25 depletion, cyclophosphamide administration, and modulating GITR function. Without wishing to be bound by theory, it is believed that reducing the number of Treg cells in a subject prior to apheresis or prior to administration of a CAR-expressing cell described herein reduces the number of unwanted immune cells (e.g., Tregs) in the tumor microenvironment and reduces the subject's risk of relapse. In one embodiment, CAR-expressing cells described herein are administered to a subject in combination with a molecule targeting GITR and/or modulating GITR functions, such as a GITR agonist and/or a GITR antibody that depletes regulatory T cells (Tregs). In one embodiment, CAR-expressing cells described herein are administered to a subject in combination with cyclophosphamide. In one embodiment, the GITR binding molecule and/or molecule modulating GITR function (e.g., GITR agonist and/or Treg depleting GITR antibodies) is administered prior to the CAR-expressing cells. For example, in one embodiment, the GITR agonist can be administered prior to apheresis of the cells. In embodiments, cyclophosphamide is administered to the subject prior to administration (e.g., infusion or re-infusion) of the CAR-expressing cell or prior to apheresis of the cells. In embodiments, cyclophosphamide and an anti-GITR antibody are administered to the subject prior to administration (e.g., infusion or re-infusion) of the CAR-expressing cell or prior to apheresis of the cells. In one embodiment, the subject has cancer (e.g., a solid cancer or a hematological cancer such as ALL or CLL). In one embodiment, the subject has CLL. In embodiments, the subject has a solid cancer, e.g., a solid cancer described herein.

In one embodiment, the combination of a CD19 CAR expressing cell described herein is administered to a subject in combination with a GITR agonist, e.g., a GITR agonist described herein. In one embodiment, the GITR agonist is administered prior to the CAR-expressing cell, e.g., CD19 CAR-expressing cells. For example, in one embodiment, the GITR agonist can be administered prior to apheresis of the cells. In one embodiment, the subject has CLL.

In one embodiment, a CAR expressing cell described herein is administered to a subject in combination with a GITR agonist, e.g., a GITR agonist described herein. In one embodiment, the GITR agonist is administered prior to the CAR-expressing cell. For example, in one embodiment, the GITR agonist can be administered prior to apheresis of the cells.

Exemplary GITR agonists include, e.g., GITR fusion proteins and anti-GITR antibodies (e.g., bivalent anti-GITR antibodies) such as, e.g., a GITR fusion protein described in U.S. Pat. No. 6,111,090, European Patent No.: 090505B1, U.S. Pat. No. 8,586,023, PCT Publication Nos.: WO 2010/003118 and 2011/090754, or an anti-GITR antibody described, e.g., in U.S. Pat. No. 7,025,962, European Patent No.: 1947183B1, U.S. Pat. Nos. 7,812,135, 8,388,967, 8,591,886, European Patent No.: EP 1866339, PCT Publication No.: WO 2011/028683, PCT Publication No.: WO 2013/039954, PCT Publication No.: WO2005/007190, PCT Publication No.: WO 2007/133822, PCT Publication No.: WO2005/055808, PCT Publication No.: WO 99/40196, PCT Publication No.: WO 2001/03720, PCT Publication No.: WO99/20758, PCT Publication No.: WO2006/083289, PCT Publication No.: WO 2005/115451, U.S. Pat. No. 7,618,632, and PCT Publication No.: WO 2011/051726.

In one embodiment, a CAR expressing cell described herein is administered to a subject in combination with a protein tyrosine phosphatase inhibitor, e.g., a protein tyrosine phosphatase inhibitor described herein. In one embodiment, the protein tyrosine phosphatase inhibitor is an SHP-1 inhibitor, e.g., an SHP-1 inhibitor described herein, such as, e.g., sodium stibogluconate. In one embodiment, the protein tyrosine phosphatase inhibitor is an SHP-2 inhibitor, e.g., an SHP-2 inhibitor described herein.

In one embodiment, a CAR-expressing cell described herein can be used in combination with a kinase inhibitor. In one embodiment, the kinase inhibitor is a CDK4 inhibitor, e.g., a CDK4 inhibitor described herein, e.g., a CD4/6 inhibitor, such as, e.g., 6-Acetyl-8-cyclopentyl-5-methyl-2-(5-piperazin-1-yl-pyridin-2-ylamino)-8H-pyrido[2,3-d]pyrimidin-7-one, hydrochloride (also referred to as palbociclib or PD0332991). In one embodiment, the kinase inhibitor is a BTK inhibitor, e.g., a BTK inhibitor described herein, such as, e.g., ibrutinib. In one embodiment, the kinase inhibitor is an mTOR inhibitor, e.g., an mTOR inhibitor described herein, such as, e.g., rapamycin, a rapamycin analog, OSI-027. The mTOR inhibitor can be, e.g., an mTORC1 inhibitor and/or an mTORC2 inhibitor, e.g., an mTORC1 inhibitor and/or mTORC2 inhibitor described herein. In one embodiment, the kinase inhibitor is a MNK inhibitor, e.g., a MNK inhibitor described herein, such as, e.g., 4-amino-5-(4-fluoroanilino)-pyrazolo[3,4-d] pyrimidine. The MNK inhibitor can be, e.g., a MNK1a, MNK1b, MNK2a and/or MNK2b inhibitor.

In one embodiment, the kinase inhibitor is a CDK4 inhibitor selected from aloisine A; flavopiridol or HMR-1275, 2-(2-chlorophenyl)-5,7-dihydroxy-8-[(3S,4R)-3-hydroxy-1-methyl-4-piperidinyl]-4-chromenone; crizotinib (PF-02341066; 2-(2-Chlorophenyl)-5,7-dihydroxy-8-[(2R,3S)-2-(hydroxymethyl)-1-methyl-3-pyrrolidinyl]-4H-1-benzopyran-4-one, hydrochloride (P276-00); 1-methyl-5-[[2-[5-(trifluoromethyl)-1H-imidazol-2-yl]-4-pyridinyl]oxy]-N-[4-(trifluoromethyl)phenyl]-1H-benzimidazol-2-amine (RAF265); indisulam (E7070); roscovitine (CYC202); palbociclib (PD0332991); dinaciclib (SCH727965); N-[5-[[(5-tert-butyloxazol-2-yl)methyl]thio]thiazol-2-yl]piperidine-4-carboxamide (BMS 387032); 4-[[9-chloro-7-(2,6-difluorophenyl)-5H-pyrimido[5,4-d][2]benzazepin-2-yl]amino]-benzoic acid (MLN8054); 5-[3-(4,6-difluoro-1H-benzimidazol-2-yl)-1H-indazol-5-yl]-N-ethyl-4-methyl-3-pyridinemethanamine (AG-024322); 4-(2,6-dichlorobenzoylamino)-1H-pyrazole-3-carboxylic acid N-(piperidin-4-yl)amide (AT7519); 4-[2-methyl-1-(1-methylethyl)-1H-imidazol-5-yl]-N-[4-(methylsulfonyl)phenyl]-2-pyrimidinamine (AZD5438); and XL281 (BMS908662).

In one embodiment, the kinase inhibitor is a CDK4 inhibitor, e.g., palbociclib (PD0332991), and the palbociclib is administered at a dose of about 50 mg, 60 mg, 70 mg, 75 mg, 80 mg, 90 mg, 100 mg, 105 mg, 110 mg, 115 mg, 120 mg, 125 mg, 130 mg, 135 mg (e.g., 75 mg, 100 mg or 125 mg) daily for a period of time, e.g., daily for 14-21 days of a 28 day cycle, or daily for 7-12 days of a 21 day cycle. In one embodiment, 1, 2, 3, 4, 5, 6, 7, 8, 9, 10, 11, 12 or more cycles of palbociclib are administered.

In embodiments, a CAR-expressing cell described herein is administered to a subject in combination with a cyclin-dependent kinase (CDK) 4 or 6 inhibitor, e.g., a CDK4 inhibitor or a CDK6 inhibitor described herein. In embodiments, a CAR-expressing cell described herein is administered to a subject in combination with a CDK4/6 inhibitor (e.g., an inhibitor that targets both CDK4 and CDK6), e.g., a CDK4/6 inhibitor described herein. In an embodiment, the subject has MCL. MCL is an aggressive cancer that is poorly responsive to currently available therapies, i.e., essentially incurable. In many cases of MCL, cyclin D1 (a regulator of CDK4/6) is expressed (e.g., due to chromosomal translocation involving immunoglobulin and Cyclin D1 genes) in MCL cells. Thus, without being bound by theory, it is thought that MCL cells are highly sensitive to CDK4/6 inhibition with high specificity (i.e., minimal effect on normal immune cells). CDK4/6 inhibitors alone have had some efficacy in treating MCL, but have only achieved partial remission with a high relapse rate. An exemplary CDK4/6 inhibitor is LEE011 (also called ribociclib).

Without being bound by theory, it is believed that administration of a CAR-expressing cell described herein with a CDK4/6 inhibitor (e.g., LEE011 or other CDK4/6 inhibitor described herein) can achieve higher responsiveness, e.g., with higher remission rates and/or lower relapse rates, e.g., compared to a CDK4/6 inhibitor alone.

In one embodiment, the kinase inhibitor is a BTK inhibitor selected from ibrutinib (PCI-32765); GDC-0834; RN-486; CGI-560; CGI-1764; HM-71224; CC-292; ONO-4059; CNX-774; and LFM-A13. In an embodiment, the BTK inhibitor does not reduce or inhibit the kinase activity of interleukin-2-inducible kinase (ITK), and is selected from GDC-0834; RN-486; CGI-560; CGI-1764; HM-71224; CC-292; ONO-4059; CNX-774; and LFM-A13.

In one embodiment, the kinase inhibitor is a BTK inhibitor, e.g., ibrutinib (PCI-32765). In embodiments, a CAR-expressing cell described herein is administered to a subject in combination with a BTK inhibitor (e.g., ibrutinib). In embodiments, a CAR-expressing cell described herein is administered to a subject in combination with ibrutinib (also called PCI-32765). The chemical name of ibrutinib is (1-[(3R)-3-[4-Amino-3-(4-phenoxyphenyl)-1H-pyrazolo[3,4-d]pyrimidin-1-yl]piperidin-1-yl]prop-2-en-1-one).

In embodiments, the subject has CLL, mantle cell lymphoma (MCL), or small lymphocytic lymphoma (SLL). For example, the subject has a deletion in the short arm of chromosome 17 (del(17p), e.g., in a leukemic cell). In other examples, the subject does not have a del(17p). In embodiments, the subject has relapsed CLL or SLL, e.g., the subject has previously been administered a cancer therapy (e.g., previously been administered one, two, three, or four prior cancer therapies). In embodiments, the subject has refractory CLL or SLL. In other embodiments, the subject has follicular lymphoma, e.g., relapse or refractory follicular lymphoma. In one embodiment, the kinase inhibitor is a BTK inhibitor, e.g., ibrutinib (PCI-32765), and the ibrutinib is administered at a dose of about 250 mg, 300 mg, 350 mg, 400 mg, 420 mg, 440 mg, 460 mg, 480 mg, 500 mg, 520 mg, 540 mg, 560 mg, 580 mg, 600 mg (e.g., 250 mg, 420 mg or 560 mg) daily for a period of time, e.g., daily for 21 day cycle, or daily for 28 day cycle. In one embodiment, 1, 2, 3, 4, 5, 6, 7, 8, 9, 10, 11, 12 or more cycles of ibrutinib are administered. In some embodiments, ibrutinib is administered in combination with rituximab. See, e.g., Burger et al. (2013) Ibrutinib In Combination With Rituximab (iR) Is Well Tolerated and Induces a High Rate Of Durable Remissions In Patients With High-Risk Chronic Lymphocytic Leukemia (CLL): New, Updated Results Of a Phase II Trial In 40 Patients, Abstract 675 presented at 55^{th} ASH Annual Meeting and Exposition, New Orleans, La. 7-10 December Without being bound by theory, it is thought that the addition of ibrutinib enhances the T cell proliferative response and may shift T cells from a T-helper-2 (Th2) to T-helper-1 (Th1) phenotype. Th1 and Th2 are phenotypes of helper T cells, with Th1 versus Th2 directing different immune response pathways. A Th1 phenotype is associated with proinflammatory responses, e.g., for killing cells, such as intracellular pathogens/viruses or cancerous cells, or perpetuating autoimmune responses. A Th2 phenotype is associated with eosinophil accumulation and anti-inflammatory responses. In some embodiments of the methods, uses, and compositions herein, the BTK inhibitor is a BTK inhibitor described in International Application WO/2015/079417, which is herein incorporated by reference in its entirety. For instance, in some embodiments, the BTK inhibitor is a compound of formula (I) or a pharmaceutically acceptable salt thereof;

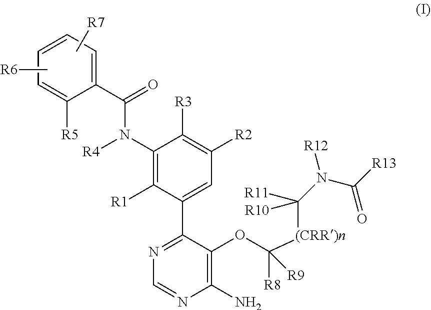

wherein,
R1 is hydrogen, C1-C6 alkyl optionally substituted by hydroxy;
R2 is hydrogen or halogen;
R3 is hydrogen or halogen;
R4 is hydrogen;
R5 is hydrogen or halogen;
or R4 and R5 are attached to each other and stand for a bond, —CH2-, —CH2-CH2-, —CH═CH—, —CH═CH—CH2-; —CH2-CH═CH—; or —CH2-CH2-CH2-;
R6 and R7 stand independently from each other for H, C1-C6 alkyl optionally substituted by hydroxyl, C3-C6 cycloalkyl optionally substituted by halogen or hydroxy, or halogen;
R8, R9, R, R′, R10 and R11 independently from each other stand for H, or C1-C6 alkyl optionally substituted by C1-C6 alkoxy; or any two of R8, R9, R, R′, R10 and R11 together with the carbon atom to which they are bound may form a 3-6 membered saturated carbocyclic ring;
R12 is hydrogen or C1-C6 alkyl optionally substituted by halogen or C1-C6 alkoxy;
or R12 and any one of R8, R9, R, R′, R10 or R11 together with the atoms to which they are bound may form a 4, 5, 6 or 7 membered azacyclic ring, which ring may optionally be substituted by halogen, cyano, hydroxyl, C1-C6 alkyl or C1-C6 alkoxy;
n is 0 or 1; and
R13 is C2-C6 alkenyl optionally substituted by C1-C6 alkyl, C1-C6 alkoxy or N,N-di-C1-C6 alkyl amino; C2-C6 alkynyl optionally substituted by C1-C6 alkyl or C1-C6 alkoxy; or C2-C6 alkylenyl oxide optionally substituted by C1-C6 alkyl.

In some embodiments, the BTK inhibitor of Formula I is chosen from: N-(3-(5-((1-Acryloylazetidin-3-yl)oxy)-6-aminopyrimidin-4-yl)-5-fluoro-2-methylphenyl)-4-cyclopropyl-2-fluorobenzamide; (E)-N-(3-(6-Amino-5-((1-(but-2-enoyl)azetidin-3-yl)oxy)pyrimidin-4-yl)-5-fluoro-2-methylphenyl)-4-cyclopropyl-2-fluorobenzamide; N-(3-(6-Amino-5-((1-propioloylazetidin-3-yl)oxy)pyrimidin-4-yl)-5-fluoro-2-methylphenyl)-4-cyclopropyl-2-fluorobenzamide; N-(3-(6-Amino-5-((1-(but-2-ynoyl)azetidin-3-yl)oxy)pyrimidin-4-yl)-5-fluoro-2-methylphenyl)-4-cyclopropyl-2-fluorobenzamide; N-(3-(5-((1-Acryloylpiperidin-4-yl)oxy)-6-aminopyrimidin-4-yl)-5-fluoro-2-methylphenyl)-4-cyclopropyl-2-fluorobenzamide; N-(3-(6-Amino-5-(2-(N-methylacrylamido)ethoxy)pyrimidin-4-yl)-5-fluoro-2-methylphenyl)-4-cyclopropyl-2-fluorobenzamide; (E)-N-(3-(6-Amino-5-(2-(N-methylbut-2-enamido)ethoxy)pyrimidin-4-yl)-5-fluoro-2-methylphenyl)-4-cyclopropyl-2-fluorobenzamide; N-(3-(6-Amino-5-(2-(N-methylpropiolamido)ethoxy)pyrimidin-4-yl)-5-fluoro-2-methylphenyl)-4-cyclopropyl-2-fluorobenzamide; (E)-N-(3-(6-Amino-5-(2-(4-methoxy-N-methylbut-2-enamido)ethoxy)pyrimidin-4-yl)-5-fluoro-2-methylphenyl)-4-cyclopropyl-2-fluorobenzamide; N-(3-(6-Amino-5-(2-(N-methylbut-2-ynamido)ethoxy)pyrimidin-4-yl)-5-fluoro-2-methylphenyl)-4-cyclopropyl-2-fluorobenzamide; N-(2-((4-Amino-6-(3-(4-cyclopropyl-2-fluorobenzamido)-5-fluoro-2-methylphenyl)pyrimidin-5-yl)oxy)ethyl)-N-methyloxirane-2-carboxamide; N-(2-((4-Amino-6-(3-(6-cyclopropyl-8-fluoro-1-oxoisoquinolin-2(1H)-yl)phenyl)pyrimidin-5-yl)oxy)ethyl)-N-methylacrylamide; N-(3-(5-(2-Acrylamidoethoxy)-6-aminopyrimidin-4-yl)-5-fluoro-2-methylphenyl)-4-cyclopropyl-2-fluorobenzamide; N-(3-(6-Amino-5-(2-(N-ethylacrylamido)ethoxy)pyrimidin-4-yl)-5-fluoro-2-methylphenyl)-4-cyclopropyl-2-fluorobenzamide; N-(3-(6-Amino-5-(2-(N-(2-fluoroethyl)acrylamido)ethoxy)pyrimidin-4-yl)-5-fluoro-2-methylphenyl)-4-cyclopropyl-2-fluorobenzamide; N-(3-(5-((1-Acrylamidocyclopropyl)methoxy)-6-aminopyrimidin-4-yl)-5-fluoro-2-methylphenyl)-4-cyclopropyl-2-fluorobenzamide; (S)—N-(3-(5-(2-Acrylamidopropoxy)-6-aminopyrimidin-4-yl)-5-fluoro-2-methylphenyl)-4-cyclopropyl-2-fluorobenzamide; (S)—N-(3-(6-Amino-5-(2-(but-2-ynamido)propoxy)pyrimidin-4-yl)-5-fluoro-2-methylphenyl)-4-cyclopropyl-2-fluorobenzamide; (S)—N-(3-(6-Amino-5-(2-(N-methylacrylamido)propoxy)pyrimidin-4-yl)-5-fluoro-2-methylphenyl)-4-cyclopropyl-2-fluorobenzamide; (S)—N-(3-(6-Amino-5-(2-(N-methylbut-2-ynamido)propoxy)pyrimidin-4-yl)-5-fluoro-2-methylphenyl)-4-cyclopropyl-2-fluorobenzamide; N-(3-(6-Amino-5-(3-(N-methylacrylamido)propoxy)pyrimidin-4-yl)-5-fluoro-2-methylphenyl)-4-cyclopropyl-2-fluorobenzamide; (S)—N-(3-(5-((1-Acryloylpyrrolidin-2-yl)methoxy)-6-aminopyrimidin-4-yl)-5-fluoro-2-methylphenyl)-4-cyclopropyl-2-fluorobenzamide; (S)—N-(3-(6-Amino-5-((1-(but-2-ynoyl)pyrrolidin-2-yl)methoxy)pyrimidin-4-yl)-5-fluoro-2-methylphenyl)-4-cyclopropyl-2-fluorobenzamide; (S)-2-(3-(5-((1-Acryloylpyrrolidin-2-yl)methoxy)-6-aminopyrimidin-4-yl)-5-fluoro-2-(hydroxymethyl)phenyl)-6-cyclopropyl-3,4-dihydroisoquinolin-1(2H)-one; N-(2-((4-Amino-6-(3-(6-cyclopropyl-1-oxo-3,4-dihydroisoquinolin-2(1H)-yl)-5-fluoro-2-(hydroxymethyl)phenyl)pyrimidin-5-yl)oxy)ethyl)-N-methylacrylamide; N-(3-(5-(((2S,4R)-1-Acryloyl-4-methoxypyrrolidin-2-yl)methoxy)-6-aminopyrimidin-4-yl)-5-fluoro-2-methylphenyl)-4-cyclopropyl-2-fluorobenzamide; N-(3-(6-Amino-5-(((2S,4R)-1-(but-2-ynoyl)-4-methoxypyrrolidin-2-yl)methoxy)pyrimidin-4-yl)-5-fluoro-2-methylphenyl)-4-cyclopropyl-2-fluorobenzamide; 2-(3-(5-(((2S,4R)-1-Acryloyl-4-methoxypyrrolidin-2-yl)methoxy)-6-aminopyrimidin-4-yl)-5-fluoro-2-(hydroxymethyl)phenyl)-6-cyclopropyl-3,4-dihydroisoquinolin-1(2H)-one; N-(3-(5-(((2S,4S)-1-Acryloyl-4-methoxypyrrolidin-2-yl)methoxy)-6-aminopyrimidin-4-yl)-5-fluoro-2-methylphenyl)-4-cyclopropyl-2-fluorobenzamide; N-(3-(6-Amino-5-(((2S,4S)-1-(but-2-ynoyl)-4-methoxypyrrolidin-2-yl)methoxy)pyrimidin-4-yl)-5-fluoro-2-methylphenyl)-4-cyclopropyl-2-fluorobenzamide; N-(3-(5-(((2S,4R)-1-Acryloyl-4-fluoropyrrolidin-2-yl)methoxy)-6-aminopyrimidin-4-yl)-5-fluoro-2-methylphenyl)-4-cyclopropyl-2-fluorobenzamide; N-(3-(6-Amino-5-(((2S,4R)-1-(but-2-ynoyl)-4-fluoropyrrolidin-2-yl)methoxy)pyrimidin-4-yl)-5-fluoro-2-methylphenyl)-4-cyclopropyl-2-fluorobenzamide; (S)—N-(3-(5-((1-Acryloylazetidin-2-yl)methoxy)-6-aminopyrimidin-4-yl)-5-fluoro-2-methylphenyl)-4-cyclopropyl-2-fluorobenzamide; (S)—N-(3-(6-Amino-5-((1-propioloylazetidin-2-yl)methoxy)pyrimidin-4-yl)-5-fluoro-2-methylphenyl)-4-cyclopropyl-2-fluorobenzamide; (S)-2-(3-(5-((1-Acryloylazetidin-2-yl)methoxy)-6-aminopyrimidin-4-yl)-5-fluoro-2-(hydroxymethyl)phenyl)-6-cyclopropyl-3,4-dihydroisoquinolin-1(2H)-one; (R)—N-(3-(5-((1-Acryloylazetidin-2-yl)methoxy)-6-aminopyrimidin-4-yl)-5-fluoro-2-methylphenyl)-4-cyclopropyl-2-fluorobenzamide; (R)—N-(3-(5-((1-Acryloylpiperidin-3-yl)methoxy)-6-aminopyrimidin-4-yl)-5-fluoro-2-methylphenyl)-4-cyclopropyl-2-fluorobenzamide; N-(3-(5-(((2R,3S)-1-Acryloyl-3-methoxypyrrolidin-2-yl)methoxy)-6-aminopyrimidin-4-yl)-5-fluoro-2-methylphenyl)-4-cyclopropyl-2-fluorobenzamide; N-(3-(5-(((2S,4R)-1-Acryloyl-4-cyanopyrrolidin-2-yl)methoxy)-6-aminopyrimidin-4-yl)-5-fluoro-2-methylphenyl)-4-cyclopropyl-2-fluorobenzamide; or N-(3-(5-(((2S,4S)-1-Acryloyl-4-cyanopyrrolidin-2-yl)methoxy)-6-aminopyrimidin-4-yl)-5-fluoro-2-methylphenyl)-4-cyclopropyl-2-fluorobenzamide.

Unless otherwise provided, the chemical terms used above in describing the BTK inhibitor of Formula I are used according to their meanings as set out in International Application WO/2015/079417, which is herein incorporated by reference in its entirety.

In one embodiment, the kinase inhibitor is an mTOR inhibitor selected from temsirolimus; ridaforolimus (1R,2R,4S)-4-[(2R)-2 [(1R,9S,12S,15R,16E,18R,19R,21R,23S,24E,26E,28Z,30S,32S,35R)-1,18-dihydroxy-19,30-dimethoxy-15,17,21,23,29,35-hexamethyl-2,3,10,14,20-pentaoxo-11,36-dioxa-4-azatricyclo[30.3.1.0^{4,9}] hexatriaconta-16,24,26,28-tetraen-12-yl]propyl]-2-methoxycyclohexyl dimethylphosphinate, also known as AP23573 and MK8669; everolimus (RAD001); rapamycin (AY22989); simapimod; (5-{2,4-bis[(3S)-3-methylmorpholin-4-yl]pyrido[2,3-d]pyrimidin-7-yl}-2-methoxyphenyl)methanol (AZD8055); 2-amino-8-[trans-4-(2-hydroxyethoxy)cyclohexyl]-6-(6-methoxy-3-pyridinyl)-4-methyl-pyrido[2,3-d]pyrimidin-7(8H)-one (PF04691502); and N²-[1,4-dioxo-4-[[4-(4-oxo-8-phenyl-4H-1-benzopyran-2-yl)morpholinium-4-yl]methoxy]butyl]-L-arginylglycyl-L-α-aspartylL-serine-, inner salt (SF1126) (SEQ ID NO: 1316); and XL765.

In one embodiment, the kinase inhibitor is an mTOR inhibitor, e.g., rapamycin, and the rapamycin is administered at a dose of about 3 mg, 4 mg, 5 mg, 6 mg, 7 mg, 8 mg, 9 mg, 10 mg (e.g., 6 mg) daily for a period of time, e.g., daily for 21 day cycle, or daily for 28 day cycle. In one embodiment, 1, 2, 3, 4, 5, 6, 7, 8, 9, 10, 11, 12 or more cycles of rapamycin are administered. In one embodiment, the kinase inhibitor is an mTOR inhibitor, e.g., everolimus and the everolimus is administered at a dose of about 2 mg, 2.5 mg, 3 mg, 4 mg, 5 mg, 6 mg, 7 mg, 8 mg, 9 mg, 10 mg, 11 mg, 12 mg, 13 mg, 14 mg, 15 mg (e.g., 10 mg) daily for a period of time, e.g., daily for 28 day cycle. In one embodiment, 1, 2, 3, 4, 5, 6, 7, 8, 9, 10, 11, 12 or more cycles of everolimus are administered.

In one embodiment, the kinase inhibitor is an MNK inhibitor selected from CGP052088; 4-amino-3-(p-fluorophenylamino)-pyrazolo[3,4-d] pyrimidine (CGP57380); cercosporamide; ETC-1780445-2; and 4-amino-5-(4-fluoroanilino)-pyrazolo[3,4-d] pyrimidine.

In embodiments, a CAR-expressing cell described herein is administered to a subject in combination with a phosphoinositide 3-kinase (PI3K) inhibitor (e.g., a PI3K inhibitor described herein, e.g., idelalisib or duvelisib) and/or rituximab. In embodiments, a CAR-expressing cell described herein is administered to a subject in combination with idelalisib and rituximab. In embodiments, a CAR-expressing cell described herein is administered to a subject in combination with duvelisib and rituximab. Idelalisib (also called GS-1101 or CAL-101; Gilead) is a small molecule that blocks the delta isoform of PI3K. The chemical name for idelalisib is (5-Fluoro-3-phenyl-2-[(1S)-1-(7H-purin-6-ylamino)propyl]-4(3H)-quinazolinone).

Duvelisib (also called IPI-145; Infinity Pharmaceuticals and Abbvie) is a small molecule that blocks PI3K-δ,γ. The chemical name for duvelisib is (8-Chloro-2-phenyl-3-[(1S)-1-(9H-purin-6-ylamino)ethyl]-1(2H)-isoquinolinone).

In embodiments, the subject has CLL. In embodiments, the subject has relapsed CLL, e.g., the subject has previously been administered a cancer therapy (e.g., previously been administered an anti-CD20 antibody or previously been administered ibrutinib). For example, the subject has a deletion in the short arm of chromosome 17 (del(17p), e.g., in a leukemic cell). In other examples, the subject does not have a del(17p). In embodiments, the subject comprises a leukemic cell comprising a mutation in the immunoglobulin heavy-chain variable-region (IgV_{H}) gene. In other embodiments, the subject does not comprise a leukemic cell comprising a mutation in the immunoglobulin heavy-chain variable-region (IgV_{H}) gene. In embodiments, the subject has a deletion in the long arm of chromosome 11 (del(11q)). In other embodiments, the subject does not have a del(11q). In embodiments, idelalisib is administered at a dosage of about 100-400 mg (e.g., 100-125, 125-150, 150-175, 175-200, 200-225, 225-250, 250-275, 275-300, 325-350, 350-375, or 375-400 mg), e.g., BID. In embodiments, duvelisib is administered at a dosage of about 15-100 mg (e.g., about 15-25, 25-50, 50-75, or 75-100 mg), e.g., twice a day. In embodiments, rituximab is administered at a dosage of about 350-550 mg/m² (e.g., 350-375, 375-400, 400-425, 425-450, 450-475, or 475-500 mg/m²), e.g., intravenously.

In one embodiment, the kinase inhibitor is a dual phosphatidylinositol 3-kinase (PI3K) and mTOR inhibitor selected from 2-Amino-8-[trans-4-(2-hydroxyethoxy)cyclohexyl]-6-(6-methoxy-3-pyridinyl)-4-methyl-pyrido[2,3-d]pyrimidin-7(8H)-one (PF-04691502); N-[4-[[4-(Dimethylamino)-1-piperidinyl]carbonyl]phenyl]-IV-[4-(4,6-di-4-morpholinyl-1,3,5-triazin-2-yl)phenyl]urea (PF-05212384, PKI-587); 2-Methyl-2-{4-[3-methyl-2-oxo-8-(quinolin-3-yl)-2,3-dihydro-1H-imidazo[4,5-c]quinolin-1-yl]phenyl}propanenitrile (BEZ-235); apitolisib (GDC-0980, RG7422); 2,4-Difluoro-N-{2-(methyloxy)-5-[4-(4-pyridazinyl)-6-quinolinyl]-3-pyridinyl}benzenesulfonamide (GSK2126458); 8-(6-methoxypyridin-3-yl)-3-methyl-1-(4-(piperazin-1-yl)-3-(trifluoromethyl)phenyl)-1H-imidazo[4,5-c]quinolin-2(3H)-one Maleic acid (NVP-BGT226); 3-[4-(4-Morpholinylpyrido[3′,2′:4,5]furo[3,2-d]pyrimidin-2-yl]phenol (PI-103); 5-(9-isopropyl-8-methyl-2-morpholino-9H-purin-6-yl)pyrimidin-2-amine (VS-5584, SB2343); and N-[2-[(3,5-Dimethoxyphenyl)amino]quinoxalin-3-yl]-4-[(4-methyl-3-methoxyphenyl)carbonyl]aminophenylsulfonamide (XL765).

In embodiments, a CAR-expressing cell described herein is administered to a subject in combination with an anaplastic lymphoma kinase (ALK) inhibitor. Exemplary ALK kinases include but are not limited to crizotinib (Pfizer), ceritinib (Novartis), alectinib (Chugai), brigatinib (also called AP26113; Ariad), entrectinib (Ignyta), PF-06463922 (Pfizer), TSR-011 (Tesaro) (see, e.g., Clinical Trial Identifier No. NCT02048488), CEP-37440 (Teva), and X-396 (Xcovery). In some embodiments, the subject has a solid cancer, e.g., a solid cancer described herein, e.g., lung cancer.

The chemical name of crizotinib is 3-[(1R)-1-(2,6-dichloro-3-fluorophenyl)ethoxy]-5-(1-piperidin-4-ylpyrazol-4-yl)pyridin-2-amine. The chemical name of ceritinib is 5-Chloro-N²-[2-isopropoxy-5-methyl-4-(4-piperidinyl)phenyl]-N⁴-[2-(isopropylsulfonyl)phenyl]-2,4-pyrimidinediamine. The chemical name of alectinib is 9-ethyl-6,6-dimethyl-8-(4-morpholinopiperidin-1-yl)-11-oxo-6,11-dihydro-5H-benzo[b]carbazole-3-carbonitrile. The chemical name of brigatinib is 5-Chloro-N²-{4-[4-(dimethylamino)-1-piperidinyl]-2-methoxyphenyl}-N⁴-[2-(dimethylphosphoryl)phenyl]-2,4-pyrimidinediamine. The chemical name of entrectinib is N-(5-(3,5-difluorobenzyl)-1H-indazol-3-yl)-4-(4-methylpiperazin-1-yl)-2-((tetrahydro-2H-pyran-4-yl)amino)benzamide. The chemical name of PF-06463922 is (10R)-7-Amino-12-fluoro-2,10,16-trimethyl-15-oxo-10,15,16,17-tetrahydro-2H-8,4-(metheno)pyrazolo[4,3-h][2,5,11]-benzoxadiazacyclotetradecine-3-carbonitrile. The chemical structure of CEP-37440 is (S)-2-((5-chloro-2-((6-(4-(2-hydroxyethyl)piperazin-1-yl)-1-methoxy-6,7,8,9-tetrahydro-5H-benzo[7]annulen-2-yl)amino)pyrimidin-4-yl)amino)-N-methylbenzamide. The chemical name of X-396 is (R)-6-amino-5-(1-(2,6-dichloro-3-fluorophenyl)ethoxy)-N-(4-(4-methylpiperazine-1-carbonyl)phenyl)pyridazine-3-carboxamide.

In one embodiment, the kinase inhibitor is an ITK inhibitor selected from ibrutinib; N-(5-(5-(4-Acetylpiperazine-1-carbonyl)-4-methoxy-2-methylphenylthio)thiazol-2-yl)-4-((3,3-dimethylbutan-2-ylamino)methyl)benzamide (BMS-509744); 7-benzyl-1-(3-(piperidin-1-yl)propyl)-2-(4-(pyridin-4-yl)phenyl)-1H-imidazo[4,5-g]quinoxalin-6(5H)-one (CTA056); R)-3-(1-(1-Acryloylpiperidin-3-yl)-4-amino-1H-pyrazolo[3,4-d]pyrimidin-3-yl)-N-(3-methyl-4-(1-methylethyl))benzamide (PF-06465469).

Drugs that inhibit either the calcium dependent phosphatase calcineurin (cyclosporine and FK506) or inhibit the p70S6 kinase that is important for growth factor induced signaling (rapamycin). (Liu et al., Cell 66:807-815, 1991; Henderson et al., Immun. 73:316-321, 1991; Bierer et al., Curr. Opin. Immun. 5:763-773, 1993) can also be used. In a further aspect, the cell compositions of the present invention may be administered to a patient in conjunction with (e.g., before, simultaneously or following) bone marrow transplantation, T cell ablative therapy using chemotherapy agents such as, fludarabine, external-beam radiation therapy (XRT), cyclophosphamide, and/or antibodies such as OKT3 or CAMPATH. In one aspect, the cell compositions of the present invention are administered following B-cell ablative therapy such as agents that react with CD20, e.g., Rituxan. For example, in one embodiment, subjects may undergo standard treatment with high dose chemotherapy followed by peripheral blood stem cell transplantation. In certain embodiments, following the transplant, subjects receive an infusion of the expanded immune cells of the present invention. In an additional embodiment, expanded cells are administered before or following surgery.

In embodiments, a CAR-expressing cell described herein is administered to a subject in combination with an indoleamine 2,3-dioxygenase (IDO) inhibitor. IDO is an enzyme that catalyzes the degradation of the amino acid, L-tryptophan, to kynurenine. Many cancers overexpress IDO, e.g., prostatic, colorectal, pancreatic, cervical, gastric, ovarian, head, and lung cancer. pDCs, macrophages, and dendritic cells (DCs) can express IDO. Without being bound by theory, it is thought that a decrease in L-tryptophan (e.g., catalyzed by IDO) results in an immunosuppressive milieu by inducing T-cell anergy and apoptosis. Thus, without being bound by theory, it is thought that an IDO inhibitor can enhance the efficacy of a CAR-expressing cell described herein, e.g., by decreasing the suppression or death of a CAR-expressing immune cell. In embodiments, the subject has a solid tumor, e.g., a solid tumor described herein, e.g., prostatic, colorectal, pancreatic, cervical, gastric, ovarian, head, or lung cancer. Exemplary inhibitors of IDO include but are not limited to 1-methyl-tryptophan, indoximod (NewLink Genetics) (see, e.g., Clinical Trial Identifier Nos. NCT01191216; NCT01792050), and INCB024360 (Incyte Corp.) (see, e.g., Clinical Trial Identifier Nos. NCT01604889; NCT01685255)

In embodiments, a CAR-expressing cell described herein is administered to a subject in combination with a modulator of myeloid-derived suppressor cells (MDSCs). MDSCs accumulate in the periphery and at the tumor site of many solid tumors. These cells suppress T cell responses, thereby hindering the efficacy of CAR-expressing cell therapy. Without being bound by theory, it is thought that administration of a MDSC modulator enhances the efficacy of a CAR-expressing cell described herein. In an embodiment, the subject has a solid tumor, e.g., a solid tumor described herein, e.g., glioblastoma. Exemplary modulators of MDSCs include but are not limited to MCS110 and BLZ945. MCS110 is a monoclonal antibody (mAb) against macrophage colony-stimulating factor (M-CSF). See, e.g., Clinical Trial Identifier No. NCT00757757. BLZ945 is a small molecule inhibitor of colony stimulating factor 1 receptor (CSF1R). See, e.g., Pyonteck et al. Nat. Med. 19(2013):1264-72.

In embodiments, a CAR-expressing cell described herein is administered to a subject in combination with a CD19 CART cell (e.g., CTL019, e.g., as described in WO2012/079000, incorporated herein by reference). In embodiments, the subject has acute myeloid leukemia (AML), e.g., a CD19 positive AML or a CD19 negative AML. In embodiments, the subject has a CD19+ lymphoma, e.g., a CD19+ Non-Hodgkin's Lymphoma (NHL), a CD19+FL, or a CD19+ DLBCL. In embodiments, the subject has a relapsed or refractory CD19+ lymphoma. In embodiments, a lymphodepleting therapy is administered to the subject prior to, concurrently with, or after administration (e.g., infusion) of CD19 CART cells. In an example, the lymphodepleting chemotherapy is administered to the subject prior to administration of CD19 CART cells. For example, the lymphodepleting chemotherapy ends 1-4 days (e.g., 1, 2, 3, or 4 days) prior to CD19 CART cell infusion. In embodiments, multiple doses of CD19 CART cells are administered, e.g., as described herein. For example, a single dose comprises about 5×10⁸ CD19 CART cells. In embodiments, a lymphodepleting chemotherapy is administered to the subject prior to, concurrently with, or after administration (e.g., infusion) of a CAR-expressing cell described herein, e.g., a non-CD19 CAR-expressing cell. In embodiments, a CD19 CART is administered to the subject prior to, concurrently with, or after administration (e.g., infusion) of a non-CD19 CAR-expressing cell, e.g., a non-CD19 CAR-expressing cell described herein.

In some embodiments, a CAR-expressing cell described herein is administered to a subject in combination with a CD19 CAR-expressing cell, e.g., CTL019, e.g., as described in WO2012/079000, incorporated herein by reference, for treatment of a disease associated with the expression of CLL-1, e.g., a cancer described herein. Without being bound by theory, it is believed that administering a CD19 CAR-expressing cell in combination with a CAR-expressing cell improves the efficacy of a CAR-expressing cell described herein by targeting early lineage cancer cells, e.g., cancer stem cells, modulating the immune response, depleting regulatory B cells, and/or improving the tumor microenvironment. For example, a CD19 CAR-expressing cell targets cancer cells that express early lineage markers, e.g., cancer stem cells and CD19-expressing cells, while the CAR-expressing cell described herein targets cancer cells that express later lineage markers, e.g., CLL-1. This preconditioning approach can improve the efficacy of the CAR-expressing cell described herein. In such embodiments, the CD19 CAR-expressing cell is administered prior to, concurrently with, or after administration (e.g., infusion) of a CAR-expressing cell described herein.

In embodiments, a CAR-expressing cell described herein also expresses a CAR targeting CD19, e.g., a CD19 CAR. In an embodiment, the cell expressing a CAR described herein and a CD19 CAR is administered to a subject for treatment of a cancer described herein, e.g., AML. In an embodiment, the configurations of one or both of the CAR molecules comprise a primary intracellular signaling domain and a costimulatory signaling domain. In another embodiment, the configurations of one or both of the CAR molecules comprise a primary intracellular signaling domain and two or more, e.g., 2, 3, 4, or 5 or more, costimulatory signaling domains. In such embodiments, the CAR molecule described herein and the CD19 CAR may have the same or a different primary intracellular signaling domain, the same or different costimulatory signaling domains, or the same number or a different number of costimulatory signaling domains. Alternatively, the CAR described herein and the CD19 CAR are configured as a split CAR, in which one of the CAR molecules comprises an antigen binding domain and a costimulatory domain (e.g., 4-1BB), while the other CAR molecule comprises an antigen binding domain and a primary intracellular signaling domain (e.g., CD3 zeta).

In some embodiments, a CAR-expressing cell described herein is administered to a subject in combination with a interleukin-15 (IL-15) polypeptide, a interleukin-15 receptor alpha (IL-15Ra) polypeptide, or a combination of both a IL-15 polypeptide and a IL-15Ra polypeptide e.g., hetIL-15 (Admune Therapeutics, LLC). hetIL-15 is a heterodimeric non-covalent complex of IL-15 and IL-15Ra. hetIL-15 is described in, e.g., U.S. Pat. No. 8,124,084, U.S. 2012/0177598, U.S. 2009/0082299, U.S. 2012/0141413, and U.S. 2011/0081311, incorporated herein by reference. In embodiments, het-IL-15 is administered subcutaneously. In embodiments, the subject has a cancer, e.g., as described herein. In some embodiments the cancer is a hematological cancer (e.g., as described herein) or, a solid cancer. In some embodiments, the solid cancer is, e.g., melanoma or colon cancer. In embodiments, the subject has a metastatic cancer.

In embodiments, a subject having a disease described herein, e.g., a hematological disorder, e.g., AML or MDS, is administered a CAR-expressing cell described herein in combination with an agent, e.g., cytotoxic or chemotherapy agent, a biologic therapy (e.g., antibody, e.g., monoclonal antibody, or cellular therapy), or an inhibitor (e.g., kinase inhibitor). In embodiments, the subject is administered a CAR-expressing cell described herein in combination with a cytotoxic agent, e.g., CPX-351 (Celator Pharmaceuticals), cytarabine, daunorubicin, vosaroxin (Sunesis Pharmaceuticals), sapacitabine (Cyclacel Pharmaceuticals), idarubicin, or mitoxantrone. CPX-351 is a liposomal formulation comprising cytarabine and daunorubicin at a 5:1 molar ratio. In embodiments, the subject is administered a CAR-expressing cell described herein in combination with a hypomethylating agent, e.g., a DNA methyltransferase inhibitor, e.g., azacitidine or decitabine. In embodiments, the subject is administered a CAR-expressing cell described herein in combination with a biologic therapy, e.g., an antibody or cellular therapy, e.g., 225Ac-lintuzumab (Actimab-A; Actinium Pharmaceuticals), IPH2102 (Innate Pharma/Bristol Myers Squibb), SGN-CD33A (Seattle Genetics), or gemtuzumab ozogamicin (Mylotarg; Pfizer). SGN-CD33A is an antibody-drug conjugate (ADC) comprising a pyrrolobenzodiazepine dimer that is attached to an anti-CD33 antibody. Actimab-A is an anti-CD33 antibody (lintuzumab) labeled with actinium. IPH2102 is a monoclonal antibody that targets killer immunoglobulin-like receptors (KIRs). In embodiments, the subject is administered a CAR-expressing cell described herein in combination a FLT3 inhibitor, e.g., sorafenib (Bayer), midostaurin (Novartis), quizartinib (Daiichi Sankyo), crenolanib (Arog Pharmaceuticals), PLX3397 (Daiichi Sankyo), AKN-028 (Akinion Pharmaceuticals), or ASP2215 (Astellas). In embodiments, the subject is administered a CAR-expressing cell described herein in combination with an isocitrate dehydrogenase (IDH) inhibitor, e.g., AG-221 (Celgene/Agios) or AG-120 (Agios/Celgene). In embodiments, the subject is administered a CAR-expressing cell described herein in combination with a cell cycle regulator, e.g., inhibitor of polo-like kinase 1 (Plk1), e.g., volasertib (Boehringer Ingelheim); or an inhibitor of cyclin-dependent kinase 9 (Cdk9), e.g., alvocidib (Tolero Pharmaceuticals/Sanofi Aventis). In embodiments, the subject is administered a CAR-expressing cell described herein in combination with a B cell receptor signaling network inhibitor, e.g., an inhibitor of B-cell lymphoma 2 (Bcl-2), e.g., venetoclax (Abbvie/Roche); or an inhibitor of Bruton's tyrosine kinase (Btk), e.g., ibrutinib (Pharmacyclics/Johnson & Johnson Janssen Pharmaceutical). In embodiments, the subject is administered a CAR-expressing cell described herein in combination with an inhibitor of M1 aminopeptidase, e.g., tosedostat (CTI BioPharmaNernalis); an inhibitor of histone deacetylase (HDAC), e.g., pracinostat (MEI Pharma); a multi-kinase inhibitor, e.g., rigosertib (Onconova Therapeutics/Baxter/SymBio); or a peptidic CXCR4 inverse agonist, e.g., BL-8040 (BioLineRx).

In another embodiment, the subjects receive an infusion of the CAR expressing cell, e.g., CD19 CAR-expressing cell, compositions of the present invention prior to transplantation, e.g., allogeneic stem cell transplant or autologous stem cell transplant, of cells. In some embodiments, CAR expressing cells transiently express the CAR, e.g., by electroporation of an mRNA CAR, whereby the expression of the antigen targeted by the CAR, e.g., CD19 is terminated prior to infusion of donor stem cells to avoid engraftment failure. In one embodiment, the subject can be administered an agent which reduces or ameliorates a side effect associated with the administration of a CAR-expressing cell. Side effects associated with the administration of a CAR-expressing cell include, but are not limited to CRS, and hemophagocytic lymphohistiocytosis (HLH), also termed Macrophage Activation Syndrome (MAS). Symptoms of CRS include high fevers, nausea, transient hypotension, hypoxia, and the like. Accordingly, the methods described herein can comprise administering a CAR-expressing cell described herein to a subject and further administering an agent to manage elevated levels of a soluble factor resulting from treatment with a CAR-expressing cell. In one embodiment, the soluble factor elevated in the subject is one or more of IFN-γ, TNFα, IL-2 and IL-6. Therefore, an agent administered to treat this side effect can be an agent that neutralizes one or more of these soluble factors. Examples of such agents include, but are not limited to a steroid (e.g., corticosteroid), an inhibitor of TNFα, and an inhibitor of IL-6. An example of a TNFα inhibitor is an anti-TNFα antibody molecule such as, infliximab, adalimumab, certolizumab pegol, and golimumab. Another example of a TNFα inhibitor is a fusion protein such as entanercept. Small molecule inhibitor of TNFα include, but are not limited to, xanthine derivatives (e.g. pentoxifylline) and bupropion. An example of an IL-6 inhibitor is an anti-IL-6 antibody molecule such as tocilizumab (toc), sarilumab, elsilimomab, CNTO 328, ALD518/BMS-945429, CNTO 136, CPSI-2364, CDP6038, VX30, ARGX-109, FE301, and FM101. In one embodiment, the anti-IL-6 antibody molecule is tocilizumab. An example of an IL-1R based inhibitor is anakinra.

### Lymphodepletion

In embodiments, lymphodepletion is performed on a subject, e.g., prior to administering one or more cells that express a CAR described herein. In embodiments, the lymphodepletion comprises administering one or more of melphalan, cytoxan, bendamustine, cyclophosphamide, and fludarabine.

In embodiments, the lymphodepletion comprises administering cyclophosphamide. In embodiments, cyclophosphamide is administered daily, e.g., for 2 or 3 days, at a dosage of about 200-700 mg/m² (e.g., 200-300, 400-600, or 450-550 mg/m², e.g., about 250 mg/m2 or 500 mg/m²), e.g., intravenously. In some embodiments, cyclophosphamide is administered at a dosage of about 250 mg/m2 per day, for 3 days. In some embodiments, cyclophosphamide is administered at a dosage of about 500 mg/m2 per day, for 2 days.

In embodiments, the lymphodepletion comprises administering fludarabine. In embodiments, fludarabine is administered daily, e.g., for 3 or 4 days, at a dosage of about 10-50 mg/m2 (e.g., 20-30, 25-40 or 25-35 mg/m2, e.g., about 25 mg/m2 or 30 mg/m2), e.g., intravenously. In some embodiments, fludarabine is administered at a dosage of about 30 mg/m2 per day, for 4 days. In some embodiments, fludarabine is administered at a dosage of about 25 mg/m2 per day, for 3 days.

In embodiments, the lymphodepletion comprises administering cyclophosphamide and fludarabine. In some embodiments, the lymphodepletion comprises administering 500 mg/m2 cyclophosphamide daily for 2 days and 30 mg/m2 fludarabine daily for 3 days. In some embodiments, the lymphodepletion comprises administering 250 mg/m2 cyclophosphamide daily for 3 days, and 25 mg/m2 fludarabine daily for 3 days. In some embodiments, the lymphodepletion begins with the administration of the first dose of fludarabine. In some embodiments, cyclophosphamide and fludarabine are administered on the same day. In some embodiments, cyclophosphamide and fludarabine are not administered on the same day. In some embodiments, the daily dosages are administered on consecutive days.

In embodiments, the lymphodepletion comprises administering bendamustine. In some embodiments, bendamustine is administered daily, e.g., twice daily, at a dosage of about 75-125 mg/m2 (e.g., 75-100 or 100-125 mg/m², e.g., about 90 mg/m²), e.g., intravenously. In some embodiments, bendamustine is administered at dosage of 90 mg/m² daily, e.g., for 2 days. In some embodiments, the subject has a cancer, e.g., a hematological cancer as described herein.

In embodiments, the lymphodepletion comprises administering bendamustine (e.g., at about 90 mg/m², e.g., daily x 2), cyclophosphamide and fludarabine (e.g., at about 200 mg/m² cyclophosphamide and about 20 mg/m² fludarabine, e.g., daily x 3), XRT and cyclophosphamide (e.g., at about 400 cGy XRT and about 1 g/m² cyclophosphamide), cyclophosphamide (e.g., about 1 g/m² or 1.2 g/m² cyclophosphamide, e.g., over 4 days), carboplatin and gemcitabine, or modified EPOCH.

In embodiments, a lymphodepleting therapy is administered to the subject prior to, concurrently with, or after administration (e.g., infusion) of CAR cells, e.g., cells described herein. In an example, the lymphodepleting therapy is administered to the subject prior to administration of CAR cells. For example, the lymphodepleting therapy ends 1-4 days (e.g., 1, 2, 3, or 4 days) prior to CAR cell infusion. In embodiments, multiple doses of CAR cells are administered, e.g., as described herein. For example, a single dose comprises about 5×10⁸ CAR cells. In embodiments, a lymphodepleting therapy is administered to the subject prior to, concurrently with, or after administration (e.g., infusion) of a CAR-expressing cell described herein.

In some embodiments, CAR-expressing cells described herein are administered to a subject in combination with a CD19 CAR-expressing cell, e.g., CTL019, e.g., as described in WO2012/079000, incorporated herein by reference, for treatment of a disease associated with the expression of cancer antigen, e.g., a cancer described herein. Without being bound by theory, it is believed that administering a CD19 CAR-expressing cell in combination with another CAR-expressing cell improves the efficacy of a CAR-expressing cell described herein by targeting early lineage cancer cells, e.g., cancer stem cells, modulating the immune response, depleting regulatory B cells, and/or improving the tumor microenvironment. For example, a CD19 CAR-expressing cell targets cancer cells that express early lineage markers, e.g., cancer stem cells and CD19-expressing cells, while some other CAR-expressing cells described herein target cancer cells that express later lineage markers. This preconditioning approach can improve the efficacy of the CAR-expressing cell described herein. In such embodiments, the CD19 CAR-expressing cell is administered prior to, concurrently with, or after administration (e.g., infusion) of the second CAR-expressing cell.

In embodiments, a CAR-expressing cell which expresses a CAR targeting a cancer antigen other than CD19 also expresses a CAR targeting CD19, e.g., a CD19 CAR. In an embodiment, the cell expressing a non-CD19 CAR and a CD19 CAR is administered to a subject for treatment of a cancer described herein, e.g., AML. In an embodiment, the configurations of one or both of the CAR molecules comprise a primary intracellular signaling domain and a costimulatory signaling domain. In another embodiment, the configurations of one or both of the CAR molecules comprise a primary intracellular signaling domain and two or more, e.g., 2, 3, 4, or 5 or more, costimulatory signaling domains. In such embodiments, the non-CD19 CAR molecule and the CD19 CAR may have the same or a different primary intracellular signaling domain, the same or different costimulatory signaling domains, or the same number or a different number of costimulatory signaling domains. Alternatively, the non-CD19 CAR and the CD19 CAR are configured as a split CAR, in which one of the CAR molecules comprises an antigen binding domain and a costimulatory domain (e.g., 4-1BB), while the other CAR molecule comprises an antigen binding domain and a primary intracellular signaling domain (e.g., CD3 zeta).

### Inhibitory Molecule Inhibitors/Checkpoint Inhibitors

In one embodiment, the subject can be administered an agent which enhances the activity of a CAR-expressing cell. For example, in one embodiment, the agent can be an agent which inhibits an inhibitory molecule, e.g., the agent is a checkpoint inhibitor. Inhibitory or checkpoint molecules, e.g., Programmed Death 1 (PD1), can, in some embodiments, decrease the ability of a CAR-expressing cell to mount an immune effector response. Examples of inhibitory molecules include PD1, PD-L1, PD-L2, CTLA4, TIM3, CEACAM (e.g., CEACAM-1, CEACAM-3 and/or CEACAM-5), LAGS, VISTA, BTLA, TIGIT, LAIR1, CD160, 2B4, CD160, 2B4, CD80, CD86, B7-H3 (CD276), B7-H4 (VTCN1), HVEM (TNFRSF14 or CD270), KIR, A2aR, MHC class I, MHC class II, GALS, adenosine, and TGF (e.g., TGF beta). In embodiments, the CAR-expressing cell described herein comprises a switch costimulatory receptor, e.g., as described in WO 2013/019615, which is incorporated herein by reference in its entirety.

The methods described herein can include administration of a CAR-expressing cell in combination with a checkpoint inhibitor. In one embodiment, the subject is a complete responder. In another embodiment, the subject is a partial responder or non-responder, and, e.g., in some embodiments, the checkpoint inhibitor is administered prior to the CAR-expressing cell, e.g., two weeks, 12 days, 10 days, 8 days, one week, 6 days, 5 days, 4 days, 3 days, 2 days or 1 day before administration of the CAR-expressing cell. In some embodiments, the checkpoint inhibitor is administered concurrently with the CAR-expressing cell.

Inhibition of an inhibitory molecule, e.g., by inhibition at the DNA, RNA or protein level, can optimize a CAR-expressing cell performance. In embodiments, an inhibitory nucleic acid, e.g., an inhibitory nucleic acid, e.g., a dsRNA, e.g., an siRNA or shRNA, or a clustered regularly interspaced short palindromic repeats (CRISPR), a transcription-activator like effector nuclease (TALEN), or a zinc finger endonuclease (ZFN), can be used to inhibit expression of an inhibitory molecule in the CAR-expressing cell. In an embodiment the inhibitor is an shRNA. In an embodiment, the inhibitory molecule is inhibited within a CAR-expressing cell. In these embodiments, a dsRNA molecule that inhibits expression of the inhibitory molecule is linked to the nucleic acid that encodes a component, e.g., all of the components, of the CAR.

In an embodiment, a nucleic acid molecule that encodes a dsRNA molecule that inhibits expression of the molecule that modulates or regulates, e.g., inhibits, T-cell function is operably linked to a promoter, e.g., a H1- or a U6-derived promoter such that the dsRNA molecule that inhibits expression of the molecule that modulates or regulates, e.g., inhibits, T-cell function is expressed, e.g., is expressed within a CAR-expressing cell. See e.g., Tiscornia G., “Development of Lentiviral Vectors Expressing siRNA,” Chapter 3, in Gene Transfer: Delivery and Expression of DNA and RNA (eds. Friedmann and Rossi). Cold Spring Harbor Laboratory Press, Cold Spring Harbor, N.Y., USA, 2007; Brummelkamp T R, et al. (2002) Science 296: 550-553; Miyagishi M, et al. (2002) Nat. Biotechnol. 19: 497-500. In an embodiment the nucleic acid molecule that encodes a dsRNA molecule that inhibits expression of the molecule that modulates or regulates, e.g., inhibits, T-cell function is present on the same vector, e.g., a lentiviral vector, that comprises a nucleic acid molecule that encodes a component, e.g., all of the components, of the CAR. In such an embodiment, the nucleic acid molecule that encodes a dsRNA molecule that inhibits expression of the molecule that modulates or regulates, e.g., inhibits, T-cell function is located on the vector, e.g., the lentiviral vector, 5′- or 3′- to the nucleic acid that encodes a component, e.g., all of the components, of the CAR. The nucleic acid molecule that encodes a dsRNA molecule that inhibits expression of the molecule that modulates or regulates, e.g., inhibits, T-cell function can be transcribed in the same or different direction as the nucleic acid that encodes a component, e.g., all of the components, of the CAR. In an embodiment the nucleic acid molecule that encodes a dsRNA molecule that inhibits expression of the molecule that modulates or regulates, e.g., inhibits, T-cell function is present on a vector other than the vector that comprises a nucleic acid molecule that encodes a component, e.g., all of the components, of the CAR. In an embodiment, the nucleic acid molecule that encodes a dsRNA molecule that inhibits expression of the molecule that modulates or regulates, e.g., inhibits, T-cell function it transiently expressed within a CAR-expressing cell. In an embodiment, the nucleic acid molecule that encodes a dsRNA molecule that inhibits expression of the molecule that modulates or regulates, e.g., inhibits, T-cell function is stably integrated into the genome of a CAR-expressing cell. In an embodiment, the molecule that modulates or regulates, e.g., inhibits, T-cell function is PD-1.

In one embodiment, the inhibitor of an inhibitory signal can be, e.g., an antibody or antibody fragment that binds to an inhibitory molecule. For example, the agent can be an antibody or antibody fragment that binds to PD1, PD-L1, PD-L2 or CTLA4 (e.g., ipilimumab (also referred to as MDX-010 and MDX-101, and marketed as Yervoy®; Bristol-Myers Squibb; Tremelimumab (IgG2 monoclonal antibody available from Pfizer, formerly known as ticilimumab, CP-675,206)). In an embodiment, the agent is an antibody or antibody fragment that binds to TIM3. In an embodiment, the agent is an antibody or antibody fragment that binds to LAG3. In an embodiment, the agent is an antibody or antibody fragment that binds to CEACAM (e.g., CEACAM-1, CEACAM-3 and/or CEACAM-5). In embodiments, the agent that enhances the activity of a CAR-expressing cell, e.g., inhibitor of an inhibitory molecule, is administered in combination with an allogeneic CAR, e.g., an allogeneic CAR described herein (e.g., described in the Allogeneic CAR section herein).

PD1 is an inhibitory member of the CD28 family of receptors that also includes CD28, CTLA-4, ICOS, and BTLA. PD1 is expressed on activated B cells, T cells and myeloid cells (Agata et al. 1996 Int. Immunol 8:765-75). Two ligands for PD1, PD-L1 and PD-L2 have been shown to downregulate T cell activation upon binding to PD1 (Freeman et a. 2000 J Exp Med 192:1027-34; Latchman et al. 2001 Nat Immunol 2:261-8; Carter et al. 2002 Eur J Immunol 32:634-43). PD-L1 is abundant in human cancers (Dong et al. 2003 J Mol Med 81:281-7; Blank et al. 2005 Cancer Immunol. Immunother 54:307-314; Konishi et al. 2004 Clin Cancer Res 10:5094). Immune suppression can be reversed by inhibiting the local interaction of PD1 with PD-L1.

Antibodies, antibody fragments, and other inhibitors of PD1, PD-L1 and PD-L2 are available in the art and may be used combination with a CD19 CAR described herein. For example, nivolumab (also referred to as BMS-936558 or MDX1106; Bristol-Myers Squibb) is a fully human IgG4 monoclonal antibody which specifically blocks PD1. Nivolumab (clone 5C4) and other human monoclonal antibodies that specifically bind to PD1 are disclosed in U.S. Pat. No. 8,008,449 and WO2006/121168. Pidilizumab (CT-011; Cure Tech) is a humanized IgG1k monoclonal antibody that binds to PD1. Pidilizumab and other humanized anti-PD1 monoclonal antibodies are disclosed in WO2009/101611. Pembrolizumab (formerly known as lambrolizumab, and also referred to as Keytruda, MK03475; Merck) is a humanized IgG4 monoclonal antibody that binds to PD1. Pembrolizumab and other humanized anti-PD1 antibodies are disclosed in U.S. Pat. No. 8,354,509 and WO2009/114335. MEDI4736 (Medimmune) is a human monoclonal antibody that binds to PDL1, and inhibits interaction of the ligand with PD1. MDPL3280A (Genentech/Roche) is a human Fc optimized IgG1 monoclonal antibody that binds to PD-L1. MDPL3280A and other human monoclonal antibodies to PD-L1 are disclosed in U.S. Pat. No. 7,943,743 and U.S Publication No.: 20120039906. Other anti-PD-L1 binding agents include YW243.55.S70 (heavy and light chain variable regions are shown in SEQ ID NOs 20 and 21 in WO2010/077634) and MDX-1 105 (also referred to as BMS-936559, and, e.g., anti-PD-L1 binding agents disclosed in WO2007/005874). AMP-224 (B7-DCIg; Amplimmune; e.g., disclosed in WO2010/027827 and WO2011/066342), is a PD-L2 Fc fusion soluble receptor that blocks the interaction between PD1 and B7-H1. Other anti-PD1 antibodies include AMP 514 (Amplimmune), among others, e.g., anti-PD1 antibodies disclosed in U.S. Pat. No. 8,609,089, US 2010028330, and/or US 20120114649.

In some embodiments, a PD1 inhibitor described herein (e.g., a PD1 antibody, e.g., a PD1 antibody described herein) is used combination with a CD19 CAR described herein to treat a disease associated with expression of CD19. In some embodiments, a PD-L1 inhibitor described herein (e.g., a PD-L1 antibody, e.g., a PD-L1 antibody described herein) is used combination with a CD19 CAR described herein to treat a disease associated with expression of CD19. In some embodiments, the CD19 CAR therapy is administered prior to, simultaneously with or after the PD-1 inhibitor. In one embodiment, the CD19 CAR therapy is administered prior to the PD-1 inhibitor. For example, one or more doses of the PD-1 inhibitor can be administered post-CD19 CAR therapy (e.g., starting 5 days to 4 months, e.g., 10 day to 3 months, e.g., 14 days to 2 months post-CD19 CAR therapy). In some embodiments, the combination of the CD19 CAR therapy and PD-1 inhibitor therapy is repeated.

The disease may be, e.g., a lymphoma such as DLBCL including primary DLBCL or secondary DLBCL. In some embodiments, the subject has, or is identified as having, at least 5%, 6%, 7%, 8%, 9%, 10%, 20%, 30%, 40%, 50%, 60%, 70%, 80%, or 90% of cancer cells, e.g., DLBCL cells, which are CD3+/PD1+. In some embodiments, the subject has, or is identified as having, substantially non-overlapping populations of CD19+ cells and PD-L1+ cells in a cancer, e.g., the cancer microenvironment. For instance, in some embodiments, less than 20%, 10%, 9%, 8%, 7%, 6%, 5%, 4%, 3%, 2%, or 1% of cells in the cancer, e.g., cancer microenvironment, are double positive for CD19 and PD-L1.

In embodiments of the CD19 CAR therapy-PD1 inhibitor therapy, the CD19 CAR therapy comprises one or more treatments with cells that express a murine CAR molecule described herein, e.g., a murine CD19 CAR molecule of Tables 3, 4 and 5 on pages 359-363 of International Application WO 2016/164731, filed Apr. 8, 2016, which is incorporated by reference in its entirety.

In another embodiment of the CD19 CAR therapy-PD1 inhibitor therapy, the CD19 CAR therapy comprises one or more treatments with cells that express a humanized CD19 CAR, e.g., a humanized CD19 CAR according to Tables 2, 4 and 5 on page 339-363 of International Application WO 2016/164731, filed Apr. 8, 2016, which is incorporated by reference in its entirety.

In some embodiments, the subject is treated with a combination of a CD19 CAR, a PD1 inhibitor, and a PD-L1 inhibitor. In some embodiments, the subject is treated with a combination of a CD19 CAR, a PD1 inhibitor, and a CD3 inhibitor. In some embodiments, the subject is treated with a combination of a CD19 CAR, a PD1 inhibitor, a PD-L1 inhibitor, and a CD3 inhibitor. Optionally, the subject has, or is identified as having, at least 5%, 6%, 7%, 8%, 9%, 10%, 20%, 30%, 40%, 50%, 60%, 70%, 80%, or 90% of cancer cells, e.g., DLBCL cells, which are CD3+/PD1+.

In some embodiments, the methods herein include a step of assaying cells in a biological sample, e.g., a sample comprising DLBCL cells, for CD3 and/or PD-1 (e.g., CD3 and/or PD-1 expression). In some embodiments, the methods include a step of assaying cells in a biological sample, e.g., a sample comprising DLBCL cells, for CD19 and/or PD-L1 (e.g., CD19 and/or PD-L1 expression). In some embodiments, the methods include, e.g., providing a sample comprising cancer cells and performing a detection step, e.g., by immunohistochemistry, for one or more of CD3, PD-1, CD19, or PD-L1. The methods may comprise a further step of recommending or administering a treatment, e.g., a treatment comprising a CD19 CAR.

In one embodiment, the anti-PD-1 antibody or fragment thereof is an anti-PD-1 antibody molecule as described in US 2015/0210769, entitled “Antibody Molecules to PD-1 and Uses Thereof,” incorporated by reference in its entirety. In one embodiment, the anti-PD-1 antibody molecule includes at least one, two, three, four, five or six CDRs (or collectively all of the CDRs) from a heavy and light chain variable region from an antibody chosen from any of BAP049-hum01, BAP049-hum02, BAP049-hum03, BAP049-hum04, BAP049-hum05, BAP049-hum06, BAP049-hum07, BAP049-hum08, BAP049-hum09, BAP049-hum10, BAP049-hum11, BAP049-hum12, BAP049-hum13, BAP049-hum14, BAP049-hum15, BAP049-hum16, BAP049-Clone-A, BAP049-Clone-B, BAP049-Clone-C, BAP049-Clone-D, or BAP049-Clone-E; or as described in Table 1 of US 2015/0210769, or encoded by the nucleotide sequence in Table 1, or a sequence substantially identical (e.g., at least 80%, 85%, 90%, 92%, 95%, 97%, 98%, 99% or higher identical) to any of the aforesaid sequences; or closely related CDRs, e.g., CDRs which are identical or which have at least one amino acid alteration, but not more than two, three or four alterations (e.g., substitutions, deletions, or insertions, e.g., conservative substitutions).

In yet another embodiment, the anti-PD-1 antibody molecule comprises at least one, two, three or four variable regions from an antibody described herein, e.g., an antibody chosen from any of BAP049-hum01, BAP049-hum02, BAP049-hum03, BAP049-hum04, BAP049-hum05, BAP049-hum06, BAP049-hum07, BAP049-hum08, BAP049-hum09, BAP049-hum10, BAP049-hum11, BAP049-hum12, BAP049-hum13, BAP049-hum14, BAP049-hum15, BAP049-hum16, BAP049-Clone-A, BAP049-Clone-B, BAP049-Clone-C, BAP049-Clone-D, or BAP049-Clone-E; or as described in Table 1 of US 2015/0210769, or encoded by the nucleotide sequence in Table 1; or a sequence substantially identical (e.g., at least 80%, 85%, 90%, 92%, 95%, 97%, 98%, 99% or higher identical) to any of the aforesaid sequences.

In one embodiment of the CD19 CAR therapy-PD1 inhibitor therapy, the PD-1 inhibitor, e.g., pembrolizumab, is administered post-CD19 CAR therapy (e.g., starting 5 days to 4 months, e.g., 10 day to 3 months, e.g., 14 days to 2 months post-CTL019 or post-CTL119 therapy, or post-a combination of CTL019 and CTL119 therapies). In embodiments, administration of the therapy is to a subject with B-ALL, e.g., relapsed or refractory B-ALL.

In yet another embodiment of the CD19 CAR therapy-PD1 inhibitor therapy, the hematologic cancer is B-ALL, e.g., relapsed or refractory B-ALL.

In one embodiment, the subject has a hematologic malignancy, e.g., B-ALL, and may not respond to the CAR T therapy or may relapse, e.g., due to poor CAR T cell persistence.

In one embodiment of the CD19 CAR therapy-PD1 inhibitor therapy, the subject shows an improved therapeutic outcome, e.g., the subject achieves one or more of partial remission, complete remission, or prolonged CAR T cell persistence, in response to the CD19 CAR therapy-PD1 inhibitor therapy, e.g., one or more cycles of the CD19 CAR therapy-PD1 inhibitor therapy.

In one embodiment of the CD19 CAR therapy-PD1 inhibitor therapy, prior to administration of the PD-1 inhibitor, the subject has relapsed or refractory B-ALL to a prior treatment with a CD19 CAR therapy, e.g., a prior treatment with one or both of CTL019 and CTL119. In some embodiments, the subject shows decreased or poor CAR T cell persistence. In some embodiments, the subject shows CD19+ relapse.

In some embodiments, the subject, e.g., a subject showing CD19+ relapse after a CD19CAR therapy, is administered a further CD19 CAR therapy, in combination with the PD-1 inhibitor, e.g., pembrolizumab. In embodiments, the further administration of the combination therapy results in an improved therapeutic outcome, e.g., the subject achieves one or more of partial remission, complete remission, or a prolonged CAR T cell persistence.

TIM3 (T cell immunoglobulin-3) also negatively regulates T cell function, particularly in IFN-g-secreting CD4+T helper 1 and CD8+T cytotoxic 1 cells, and plays a critical role in T cell exhaustion. Inhibition of the interaction between TIM3 and its ligands, e.g., galectin-9 (Gal9), phosphatidylserine (PS), and HMGB1, can increase immune response. Antibodies, antibody fragments, and other inhibitors of TIM3 and its ligands are available in the art and may be used combination with a CD19 CAR described herein. For example, antibodies, antibody fragments, small molecules, or peptide inhibitors that target TIM3 binds to the IgV domain of TIM3 to inhibit interaction with its ligands. Antibodies and peptides that inhibit TIM3 are disclosed in WO2013/006490 and US20100247521. Other anti-TIM3 antibodies include humanized versions of RMT3-23 (disclosed in Ngiow et al., 2011, Cancer Res, 71:3540-3551), and clone 8B.2C12 (disclosed in Monney et al., 2002, Nature, 415:536-541). Bi-specific antibodies that inhibit TIM3 and PD-1 are disclosed in US20130156774.

In one embodiment, the anti-TIM3 antibody or fragment thereof is an anti-TIM3 antibody molecule as described in US 2015/0218274, entitled “Antibody Molecules to TIM3 and Uses Thereof,” incorporated by reference in its entirety. In one embodiment, the anti-TIM3 antibody molecule includes at least one, two, three, four, five or six CDRs (or collectively all of the CDRs) from a heavy and light chain variable region from an antibody chosen from any of ABTIM3, ABTIM3-hum01, ABTIM3-hum02, ABTIM3-hum03, ABTIM3-hum04, ABTIM3-hum05, ABTIM3-hum06, ABTIM3-hum07, ABTIM3-hum08, ABTIM3-hum09, ABTIM3-hum10, ABTIM3-hum11, ABTIM3-hum12, ABTIM3-hum13, ABTIM3-hum14, ABTIM3-hum15, ABTIM3-hum16, ABTIM3-hum17, ABTIM3-hum18, ABTIM3-hum19, ABTIM3-hum20, ABTIM3-hum21, ABTIM3-hum22, ABTIM3-hum23; or as described in Tables 1-4 of US 2015/0218274; or encoded by the nucleotide sequence in Tables 1-4; or a sequence substantially identical (e.g., at least 80%, 85%, 90%, 92%, 95%, 97%, 98%, 99% or higher identical) to any of the aforesaid sequences, or closely related CDRs, e.g., CDRs which are identical or which have at least one amino acid alteration, but not more than two, three or four alterations (e.g., substitutions, deletions, or insertions, e.g., conservative substitutions).

In yet another embodiment, the anti-TIM3 antibody molecule comprises at least one, two, three or four variable regions from an antibody described herein, e.g., an antibody chosen from any of ABTIM3, ABTIM3-hum01, ABTIM3-hum02, ABTIM3-hum03, ABTIM3-hum04, ABTIM3-hum05, ABTIM3-hum06, ABTIM3-hum07, ABTIM3-hum08, ABTIM3-hum09, ABTIM3-hum10, ABTIM3-hum11, ABTIM3-hum12, ABTIM3-hum13, ABTIM3-hum14, ABTIM3-hum15, ABTIM3-hum16, ABTIM3-hum17, ABTIM3-hum18, ABTIM3-hum19, ABTIM3-hum20, ABTIM3-hum21, ABTIM3-hum22, ABTIM3-hum23; or as described in Tables 1-4 of US 2015/0218274; or encoded by the nucleotide sequence in Tables 1-4; or a sequence substantially identical (e.g., at least 80%, 85%, 90%, 92%, 95%, 97%, 98%, 99% or higher identical) to any of the aforesaid sequences.

In other embodiments, the agent which enhances the activity of a CAR-expressing cell is a CEACAM inhibitor (e.g., CEACAM-1, CEACAM-3, and/or CEACAM-5 inhibitor). In one embodiment, the inhibitor of CEACAM is an anti-CEACAM antibody molecule. Exemplary anti-CEACAM-1 antibodies are described in WO 2010/125571, WO 2013/082366 WO 2014/059251 and WO 2014/022332, e.g., a monoclonal antibody 34B1, 26H7, and 5F4; or a recombinant form thereof, as described in, e.g., US 2004/0047858, U.S. Pat. No. 7,132,255 and WO 99/052552. In other embodiments, the anti-CEACAM antibody binds to CEACAM-5 as described in, e.g., Zheng et al. PLoS One. 2010 Sep. 2; 5(9). pii: e12529 (DOI:10:1371/journal.pone.0021146), or crossreacts with CEACAM-1 and CEACAM-5 as described in, e.g., WO 2013/054331 and US 2014/0271618.

Without wishing to be bound by theory, carcinoembryonic antigen cell adhesion molecules (CEACAM), such as CEACAM-1 and CEACAM-5, are believed to mediate, at least in part, inhibition of an anti-tumor immune response (see e.g., Markel et al. J Immunol. 2002 Mar. 15; 168(6):2803-10; Markel et al. J Immunol. 2006 Nov. 1; 177(9):6062-71; Markel et al. Immunology. 2009 February; 126(2):186-200; Markel et al. Cancer Immunol Immunother. 2010 February; 59(2):215-30; Ortenberg et al. Mol Cancer Ther. 2012 June; 11(6):1300-10; Stern et al. J Immunol. 2005 Jun. 1; 174(11):6692-701; Zheng et al. PLoS One. 2010 Sep. 2; 5(9). pii: e12529). For example, CEACAM-1 has been described as a heterophilic ligand for TIM-3 and as playing a role in TIM-3-mediated T cell tolerance and exhaustion (see e.g., WO 2014/022332; Huang, et al. (2014) Nature doi:10.1038/nature13848). In embodiments, co-blockade of CEACAM-1 and TIM-3 has been shown to enhance an anti-tumor immune response in xenograft colorectal cancer models (see e.g., WO 2014/022332; Huang, et al. (2014), supra). In other embodiments, co-blockade of CEACAM-1 and PD-1 reduce T cell tolerance as described, e.g., in WO 2014/059251. Thus, CEACAM inhibitors can be used with the other immunomodulators described herein (e.g., anti-PD-1 and/or anti-TIM-3 inhibitors) to enhance an immune response against a cancer, e.g., a melanoma, a lung cancer (e.g., NSCLC), a bladder cancer, a colon cancer, an ovarian cancer, and other cancers as described herein.

LAG3 (lymphocyte activation gene-3 or CD223) is a cell surface molecule expressed on activated T cells and B cells that has been shown to play a role in CD8+ T cell exhaustion. Antibodies, antibody fragments, and other inhibitors of LAG3 and its ligands are available in the art and may be used combination with a CD19 CAR described herein. For example, BMS-986016 (Bristol-Myers Squib) is a monoclonal antibody that targets LAG3. IMP701 (Immutep) is an antagonist LAG3 antibody and IMP731 (Immutep and GlaxoSmithKline) is a depleting LAG3 antibody. Other LAG3 inhibitors include IMP321 (Immutep), which is a recombinant fusion protein of a soluble portion of LAG3 and Ig that binds to MHC class II molecules and activates antigen presenting cells (APC). Other antibodies are disclosed, e.g., in WO2010/019570.

In one embodiment, the anti-LAG3 antibody or fragment thereof is an anti-LAG3 antibody molecule as described in US 2015/0259420, entitled “Antibody Molecules to LAG3 and Uses Thereof,” incorporated by reference in its entirety. In one embodiment, the anti-LAG3 antibody molecule includes at least one, two, three, four, five or six CDRs (or collectively all of the CDRs) from a heavy and light chain variable region from an antibody chosen from any of BAP050-hum01, BAP050-hum02, BAP050-hum03, BAP050-hum04, BAP050-hum05, BAP050-hum06, BAP050-hum07, BAP050-hum08, BAP050-hum09, BAP050-hum10, BAP050-hum11, BAP050-hum12, BAP050-hum13, BAP050-hum14, BAP050-hum15, BAP050-hum16, BAP050-hum17, BAP050-hum18, BAP050-hum19, BAP050-hum20, huBAP050(Ser) (e.g., BAP050-hum01-Ser, BAP050-hum02-Ser, BAP050-hum03-Ser, BAP050-hum04-Ser, BAP050-hum05-Ser, BAP050-hum06-Ser, BAP050-hum07-Ser, BAP050-hum08-Ser, BAP050-hum09-Ser, BAP050-hum10-Ser, BAP050-hum11-Ser, BAP050-hum12-Ser, BAP050-hum13-Ser, BAP050-hum14-Ser, BAP050-hum15-Ser, BAP050-hum18-Ser, BAP050-hum19-Ser, or BAP050-hum20-Ser), BAP050-Clone-F, BAP050-Clone-G, BAP050-Clone-H, BAP050-Clone-I, or BAP050-Clone-J; or as described in Table 1 of US 2015/0259420; or encoded by the nucleotide sequence in Table 1; or a sequence substantially identical (e.g., at least 80%, 85%, 90%, 92%, 95%, 97%, 98%, 99% or higher identical) to any of the aforesaid sequences, or closely related CDRs, e.g., CDRs which are identical or which have at least one amino acid alteration, but not more than two, three or four alterations (e.g., substitutions, deletions, or insertions, e.g., conservative substitutions).

In yet another embodiment, the anti-LAG3 antibody molecule comprises at least one, two, three or four variable regions from an antibody described herein, e.g., an antibody chosen from any of BAP050-hum01, BAP050-hum02, BAP050-hum03, BAP050-hum04, BAP050-hum05, BAP050-hum06, BAP050-hum07, BAP050-hum08, BAP050-hum09, BAP050-hum10, BAP050-hum11, BAP050-hum12, BAP050-hum13, BAP050-hum14, BAP050-hum15, BAP050-hum16, BAP050-hum17, BAP050-hum18, BAP050-hum19, BAP050-hum20, huBAP050(Ser) (e.g., BAP050-hum01-Ser, BAP050-hum02-Ser, BAP050-hum03-Ser, BAP050-hum04-Ser, BAP050-hum05-Ser, BAP050-hum06-Ser, BAP050-hum07-Ser, BAP050-hum08-Ser, BAP050-hum09-Ser, BAP050-hum10-Ser, BAP050-hum11-Ser, BAP050-hum12-Ser, BAP050-hum13-Ser, BAP050-hum14-Ser, BAP050-hum15-Ser, BAP050-hum18-Ser, BAP050-hum19-Ser, or BAP050-hum20-Ser), BAP050-Clone-F, BAP050-Clone-G, BAP050-Clone-H, BAP050-Clone-I, or BAP050-Clone-J; or as described in Table 1 of US 2015/0259420; or encoded by the nucleotide sequence in Tables 1; or a sequence substantially identical (e.g., at least 80%, 85%, 90%, 92%, 95%, 97%, 98%, 99% or higher identical) to any of the aforesaid sequences.

In embodiments, the subject is administered an additional agent (in further combination with a CAR-expressing cell, e.g., a CD19 CAR-expressing cell), where the additional agent is an inhibitor of an inhibitory molecule, e.g., checkpoint molecule, e.g., PD-1, PD-L1, CTLA4, TIM3, CEACAM (e.g., CEACAM-1, CEACAM-3 and/or CEACAM-5), LAGS, VISTA, BTLA, TIGIT, LAIR1, CD160, 2B4, CD80, CD86, B7-H3 (CD276), B7-H4 (VTCN1), HVEM (TNFRSF14 or CD270), KIR, A2aR, MHC class I, MHC class II, GALS, adenosine, or TGF beta. In embodiments, the additional agent is an inhibitor of PD-L1, e.g., FAZ053 (a hIgG4 humanized anti-PD-L1 monoclonal antibody), MPDL3280A, durvalumab (DEMI-4736), avelumab (MSB-0010718C), or BMS-936559. In embodiments, the additional agent is an additional inhibitor of PD-1, e.g., pembrolizumab, nivolumab, PDR001, MEDI-0680 (AMP-514), AMP-224, REGN-2810, or BGB-A317. In embodiments, the additional agent is an inhibitor of CTLA-4, e.g., ipilimumab. In embodiments, the additional agent is an inhibitor of LAG-3, e.g., LAG525 (a hIgG4 humanized anti-LAG-3 monoclonal antibody). In embodiments, the additional agent is an inhibitor of TIM-3, e.g., MBG453 (a hIgG4 humanized anti-TIM-3 monoclonal antibody). In embodiments, the additional agent is an inhibitor of the enzyme, B-Raf, e.g., dabrafenib (GSK2118436; N-{3-[5-(2-aminopyrimidin-4-yl)-2-tert-butyl-1,3-thiazol-4-yl]-2-fluorophenyl}-2,6-difluorobenzenesulfonamide). In embodiments, the additional agent is an inhibitor of MEK1 and/or MEK2, e.g., trametinib (N-(3-{3-Cyclopropyl-5-[(2-fluoro-4-iodophenyl)amino]-6,8-dimethyl-2,4,7-trioxo-3,4,6,7-tetrahydropyrido[4,3-d]pyrimidin-1(2H)-yl}phenyl)acetamide). In embodiments, the additional agent comprises dabrafenib and trametinib. In embodiments, the additional agent is an inhibitor of GITR, e.g., GWN323. In embodiments, the additional agent is an agonist of STING (Stimulator of Interferon Genes), e.g., MIW815. In embodiments, the additional agent is an IL-15 agonist, e.g., NIZ985. In embodiments, the additional agent an inhibitor of adenosine receptor, e.g., NIR178. In embodiments, the additional agent is an inhibitor of macrophage colony stimulating factor (CSF-1), e.g., MCS110. In embodiments, the additional agent is an inhibitor of cMet, e.g., INC280. In embodiments, the additional agent is an inhibitor of porcupine (PORCN), e.g., WNT974. In embodiments, the additional agent is a histone deacetylase inhibitor, e.g., panobinost. In embodiments, the additional agent is an mTOR inhibitor, e.g., everolimus. In embodiments, the additional agent is a second mitochondrial-derived activator of caspases (SMAC) mimetic and/or an inhibitor of IAP (inhibiotor of apoptosis protein) family of proteins, e.g., LCL161. In embodiments, the additional agent is an inhibitor epidermal growth factor receptor (EGFR), e.g., EGF816. In embodiments, the additional agent is an inhibitor of IL-17, e.g., CJM112. In embodiments, the additional agent is an inhibitor of IL-1beta, e.g., ILARIS.

While not wishing to be bound by theory, in some embodiments, a tumor microenvironment is not conducive to CART cells attacking cancer cells, due to direct or indirect inhibitory effects exerted by the presence of PD-L1+ expressing cells or PD1+ T cells within the tumor microenvironment. More specifically, a tumor microenvironment can comprise tumor cells (which are generally CD19+), immune effector cells (which can be CD3+ T cells and can be PD1+ or PD1−, and which can be endogenous cells or CAR-expressing cells), and activated myeloid cells (which are generally PD-L1+). PD1+ T cells can create a “barrier” around the tumor microenvironment by preventing entry of CART cells the tumor. According to the non-limiting theory herein, pre-administration of a PD1 inhibitor and/or PD-L1 inhibitor makes the tumor microenvironment more favorable to entry of CAR-expressing cells into the tumor microenvironment and effectively clear the target positive cancer cells.

Accordingly, in certain aspects, the present disclosure provides methods of combination therapy comprising administering to a subject a cell that expresses a CAR molecule that binds CD19, e.g., a CD19 CAR, in combination with a PD1 inhibitor, a PD-L1 inhibitor, or both. In some embodiments, the PD1 inhibitor and/or PD-L1 inhibitor is administered before the CAR therapy. In other embodiments, the PD1 inhibitor and/or PD-L1 inhibitor is administered concurrently with or after the CAR therapy. In some aspects, the subject is a subject having a disease associated with expression of CD19, e.g., a hematologic malignancy, e.g., a leukemia or lymphoma, e.g., DLBCL, e.g. primary DLBCL. In some embodiments, the patient has, or is identified as having, elevated levels of PD1, PDL1, or CD3, or any combination thereof. In some embodiments, the patient has, or is identified as having, or at least 5%, 6%, 7%, 8%, 9%, 10%, 20%, 30%, 40%, 50%, 60%, 70%, 80%, or 90% of DLBCL cells which are positive for CD3 and PD1.

Also provided herein are methods for monitoring the efficacy of a CAR therapy, e.g., a CD19 CAR therapy. CAR-expressing cells can be administered to a patient's bloodstream with the intent that the cells home to a tumor cell, e.g., infiltrate a tumor. Accordingly, in some embodiments, the method comprises assaying a tumor sample for the presence of CAR-expressing cells. In embodiments, the method comprises detecting a tumor marker, e.g., CD19. In embodiments, the method comprises detecting a marker of a CAR-expressing cell, e.g., a CAR construct or nucleic acid encoding the CAR construct. In embodiments, the method further comprises detecting a T cell marker, e.g., CD3. In some aspects, the subject is a subject having a disease associated with expression of CD19, e.g., a hematologic malignancy, e.g., a leukemia or lymphoma, e.g., DLBCL, e.g. primary DLBCL. In some embodiments, if the CAR-expressing cells show poor infiltration of the tumor, the subject is identified as at an elevated risk of relapse compared to a subject with good infiltration of the tumor. In some embodiments, if the CAR-expressing cells show poor infiltration of the tumor, the subject is administered a PD1 inhibitor and/or PD-L1 inhibitor, e.g., in combination with a second dose of CAR-expressing cells.

In some embodiments, the agent which enhances the activity of a CAR-expressing cell can be, e.g., a fusion protein comprising a first domain and a second domain, wherein the first domain is an inhibitory molecule, or fragment thereof, and the second domain is a polypeptide that is associated with a positive signal, e.g., a polypeptide comprising an intracellular signaling domain as described herein. In some embodiments, the polypeptide that is associated with a positive signal can include a costimulatory domain of CD28, CD27, ICOS, e.g., an intracellular signaling domain of CD28, CD27 and/or ICOS, and/or a primary signaling domain, e.g., of CD3 zeta, e.g., described herein. In one embodiment, the fusion protein is expressed by the same cell that expressed the CAR. In another embodiment, the fusion protein is expressed by a cell, e.g., a T cell that does not express an anti-CD19 CAR.

In an embodiment, the method further comprises administering a checkpoint inhibitor. In embodiments, the subject receives a pre-treatment of with an agent, e.g., an mTOR inhibitor, and/or a checkpoint inhibitor, prior to the initiation of a CART therapy. In embodiments, the subject receives concurrent treatment with an agent, e.g., an mTOR inhibitor, and/or a checkpoint inhibitor. In embodiments, the subject receives treatment with an agent, e.g., an mTOR inhibitor, and/or a checkpoint inhibitor, post-CART therapy.

In embodiments, the determined level or determined characteristic is acquired before, at the same time, or during a course of CART therapy.

In one embodiment, the agent which enhances activity of a CAR-expressing cell described herein is miR-17-92.

In one embodiment, the agent which enhances activity of a CAR-described herein is a cytokine. Cytokines have important functions related to T cell expansion, differentiation, survival, and homeostasis. Cytokines that can be administered to the subject receiving a CAR-expressing cell described herein include: IL-2, IL-4, IL-7, IL-9, IL-15, IL-18, and IL-21, or a combination thereof. In embodiments, the cytokine administered is IL-7, IL-15, or IL-21, or a combination thereof. The cytokine can be administered once a day or more than once a day, e.g., twice a day, three times a day, or four times a day. The cytokine can be administered for more than one day, e.g. the cytokine is administered for 2 days, 3 days, 4 days, 5 days, 6 days, 1 week, 2 weeks, 3 weeks, or 4 weeks. For example, the cytokine is administered once a day for 7 days.

In embodiments, the cytokine is administered in combination with CAR-expressing cells. The cytokine can be administered simultaneously or concurrently with the CAR-expressing cells, e.g., administered on the same day. The cytokine may be prepared in the same pharmaceutical composition as the CAR-expressing cells, or may be prepared in a separate pharmaceutical composition. Alternatively, the cytokine can be administered shortly after administration of the CAR-expressing T cells, e.g., 1 day, 2 days, 3 days, 4 days, 5 days, 6 days, or 7 days after administration of the CAR-expressing cells. In embodiments where the cytokine is administered in a dosing regimen that occurs over more than one day, the first day of the cytokine dosing regimen can be on the same day as administration with the CAR-expressing cells, or the first day of the cytokine dosing regimen can be 1 day, 2 days, 3 days, 4 days, 5 days, 6 days, or 7 days after administration of the CAR-expressing T cells. In one embodiment, on the first day, the CAR-expressing cells are administered to the subject, and on the second day, a cytokine is administered once a day for the next 7 days. In an embodiment, the cytokine to be administered in combination with the CAR-expressing cells is IL-7, IL-15, and/or IL-21.

In other embodiments, the cytokine is administered a sufficient period of time after administration of the CAR-expressing cells, e.g., at least 2 weeks, 3 weeks, 4 weeks, 6 weeks, 8 weeks, 10 weeks, 12 weeks, 4 months, 5 months, 6 months, 7 months, 8 months, 9 months, 10 months, 11 months, or 1 year or more after administration of CAR-expressing cells. In one embodiment, the cytokine is administered after assessment of the subject's response to the CAR-expressing cells. For example, the subject is administered CAR-expressing cells according to the dosage and regimens described herein. The response of the subject to CART therapy is assessed at 2 weeks, 3 weeks, 4 weeks, 6 weeks, 8 weeks, 10 weeks, 12 weeks, 4 months, 5 months, 6 months, 7 months, 8 months, 9 months, 10 months, 11 months, or 1 year or more after administration of CAR-expressing cells, using any of the methods described herein, including inhibition of tumor growth, reduction of circulating tumor cells, or tumor regression. Subjects that do not exhibit a sufficient response to CART therapy can be administered a cytokine. Administration of the cytokine to the subject that has sub-optimal response to the CART therapy improves CART efficacy and/or anti-tumor activity. In an embodiment, the cytokine administered after administration of CAR-expressing cells is IL-7.

The structure of the active compounds identified by code numbers, generic or trade names may be taken from the actual edition of the standard compendium “The Merck Index” or from databases, e.g. Patents International (e.g. IMS World Publications).

The above-mentioned compounds, which can be used in combination with a compound of the present invention, can be prepared and administered as described in the art, such as in the documents cited above.

In one embodiment, the present invention provides pharmaceutical compositions comprising at least one compound of the present invention (e.g., a compound of the present invention) or a pharmaceutically acceptable salt thereof together with a pharmaceutically acceptable carrier suitable for administration to a human or animal subject, either alone or together with other anti-cancer agents.

In one embodiment, the present invention provides methods of treating human or animal subjects suffering from a cellular proliferative disease, such as cancer. The present invention provides methods of treating a human or animal subject in need of such treatment, comprising administering to the subject a therapeutically effective amount of a compound of the present invention (e.g., a compound of the present invention) or a pharmaceutically acceptable salt thereof, either alone or in combination with other anti-cancer agents.

In particular, compositions will either be formulated together as a combination therapeutic or administered separately.

In combination therapy, the compound of the present invention and other anti-cancer agent(s) may be administered either simultaneously, concurrently or sequentially with no specific time limits, wherein such administration provides therapeutically effective levels of the two compounds in the body of the patient.

In a embodiment, the compound of the present invention and the other anti-cancer agent(s) is generally administered sequentially in any order by infusion or orally. The dosing regimen may vary depending upon the stage of the disease, physical fitness of the patient, safety profiles of the individual drugs, and tolerance of the individual drugs, as well as other criteria well-known to the attending physician and medical practitioner(s) administering the combination. The compound of the present invention and other anti-cancer agent(s) may be administered within minutes of each other, hours, days, or even weeks apart depending upon the particular cycle being used for treatment. In addition, the cycle could include administration of one drug more often than the other during the treatment cycle and at different doses per administration of the drug.

In another aspect of the present invention, kits that include one or more compound of the present invention and a combination partner as disclosed herein are provided. Representative kits include (a) a compound of the present invention or a pharmaceutically acceptable salt thereof, (b) at least one combination partner, e.g., as indicated above, whereby such kit may comprise a package insert or other labeling including directions for administration.

A compound of the present invention may also be used to advantage in combination with known therapeutic processes, for example, the administration of hormones or especially radiation. A compound of the present invention may in particular be used as a radiosensitizer, especially for the treatment of tumors which exhibit poor sensitivity to radiotherapy.

Combination with a Low, Immune Enhancing, Dose of an mTOR Inhibitor

Methods described herein use low, immune enhancing, doses of mTOR inhibitors, e.g., allosteric mTOR inhibitors, including rapalogs such as RAD001. Administration of a low, immune enhancing, dose of an mTOR inhibitor (e.g., a dose that is insufficient to completely suppress the immune system, but sufficient to improve immune function) can optimize the performance of immune effector cells, e.g., T cells or CAR-expressing cells, in the subject. Methods for measuring mTOR inhibition, dosages, treatment regimens, and suitable pharmaceutical compositions are described in U.S. Patent Application No. 2015/0140036, hereby incorporated by reference.

### Methods and Biomarkers for Evaluating CAR-Effectiveness or Sample Suitability

The present disclosure provides, among other things, gene signatures that indicate whether a cancer patient treated with a CAR therapy is likely to relapse, or has relapsed. Without wishing to be bound by theory, an experimental basis for this gene signature is set out in Example 12 on pages 528-532 of International Application WO 2016/164731, filed Apr. 8, 2016, which is incorporated by reference in its entirety.

In an embodiment, novel transcriptional gene signatures described e.g., in Table 29 (on page 530 International Application WO 2016/164731, filed Apr. 8, 2016, which is incorporated by reference in its entirety), are used to enable manufactured product improvements, thereby reducing the likelihood of patient relapse. In an embodiment, gene signatures described herein are used to modify therapeutic application of manufactured product, thereby reducing the likelihood of patient relapse.

In an embodiment, gene signatures described e.g., in Table 29 (on page 530 International Application WO 2016/164731, filed Apr. 8, 2016, which is incorporated by reference in its entirety) are identified in a subject prior to treatment with a CAR-expressing cell, e.g., CART treatment (e.g., a CART19 treatment, e.g., CTL019 therapy) that predict relapse to CAR treatment. In an embodiment, gene signatures described herein are identified in an apheresis sample or bone marrow sample. In an embodiment, gene signatures described herein are identified in a manufactured CAR-expressing cell product, e.g., CART product (e.g., a CART19 product, e.g., CTL019) prior to infusion.

In embodiments, a method of using the compositions described herein comprises assaying a gene signature that indicates whether a subject treated with the cell is likely to relapse, or has relapsed. In embodiments, the method comprises assaying the gene signature in the cell prior to infusion into the subject. In embodiments, the method further comprises decreasing the T_{REG} signature of a population of cells comprising the transduced cell. In embodiments, decreasing the T_{REG} signature comprises performing CD25-depletion on the population of cells.

In embodiments, a method comprises assaying a gene signature that indicates whether the subject is likely to relapse, or has relapsed. In embodiments, the method comprises assaying a gene signature in a subject prior to treatment with a CAR-expressing cell, e.g., CART treatment (e.g., a CART19 treatment, e.g., CTL019 therapy) that predicts relapse to CAR treatment. In embodiments, the level of one or more markers is the level of at least 2, 3, 4, 5, 6, 7, 8, 9, or 10 markers listed in Table 29 (on page 530 International Application WO 2016/164731, filed Apr. 8, 2016, which is incorporated by reference in its entirety). In embodiments, the level of the marker comprises an mRNA level or a level of a soluble protein.

This disclosure also provides evidence, for instance in Example 12, on pages 528-532 of International Application WO 2016/164731, filed Apr. 8, 2016, which is incorporated by reference in its entirety, that (without wishing to be bound by theory) decreasing the T_{REG} signature in the patient prior to apheresis or during manufacturing of the CART product reduces the risk of patient relapse.

In an embodiment, a patient is pre-treated with one or more therapies that reduce T_{REG} cells prior to collection of cells for CAR product manufacturing, e.g., CART product manufacturing, thereby reducing the risk of patient relapse to CAR-expressing cell treatment (e.g., CTL019 treatment). Methods of decreasing T_{REG} cells include, but are not limited to, cyclophosphamide, anti-GITR antibody, CD25-depletion, and combinations thereof.

In an embodiment, a patient is pre-treated with cyclophosphamide or an anti-GITR antibody prior to collection of cells for CAR-expressing cell product manufacturing, thereby reducing the risk of patient relapse to CAR-expressing cell treatment (e.g., CTL019 treatment).

In an embodiment, the CAR-expressing cell manufacturing process is modified to deplete T_{REG} cells prior to manufacturing of the CAR-expressing cell product (e.g., a CTL019 product). In an embodiment, CD25-depletion is used to deplete T_{REG} cells prior to manufacturing of the CAR-expressing cell product (e.g., a CTL019 product).

In an embodiment, after treating a patient or a CAR-expressing cell product with a treatment that reduces T_{REG} cells, the patient is treated with a combination therapy. The combination therapy may comprise, e.g., a CD19 inhibitor such as a CD19 CAR-expressing cell.

In an embodiment, a patient is assayed for the level of T_{REG} cells in a patient sample, e.g., a sample comprising cancer cells and/or a sample representing a tumor microenvironment. In an embodiment, this information is used to determine a course of treatment for the patient. For instance, in an embodiment, if the patient is identified as having elevated levels of T_{REG} cells compared to a control, the therapy comprises administering a treatment other than a CAR-expressing cell. For instance, the therapy may comprise administration of an antibody molecule, administration of a small molecule therapeutic, surgery, or radiation therapy, or any combination thereof. This therapy may target one or more B-cell antigens.

In embodiments, the characteristic of CD19 is a mutation in exon 2, e.g., a mutation causing a frameshift or a premature stop codon or both. In embodiments, the level of T_{REG} cells is determined by staining a sample for a marker expressed by T_{REG} cells. In embodiments, the level of T_{REG} cells is the level of Treg cells in a relevant location in the subject's body, e.g., in a cancer microenvironment.

In an embodiment, a relapser is a patient having, or who is identified as having, an increased level of expression (e.g., increase in RNA levels) of one or more of (e.g., 2, 3, 4, or all of) the following genes, compared to non relapsers: MIR199A1, MIR1203, uc021ovp, ITM2C, and HLA-DQB1 and/or a decreased levels of expression (e.g., decrease in RNA levels) of one or more of (e.g., 2, 3, 4, 5, 6, 7, 8, 9, 10, 11, or all of) the following genes, compared to non relapsers: PPIAL4D, TTTY10, TXLNG2P, MIR4650-1, KDM5D, USP9Y, PRKY, RPS4Y2, RPS4Y1, NCRNA00185, SULT1E1, and EIF1AY.

In another aspect, the invention features a method of evaluating or monitoring the effectiveness of a CAR-expressing cell therapy, in a subject (e.g., a subject having a cancer), or the suitability of a sample (e.g., an apheresis sample) for a CAR therapy, e.g., therapy including administration of a low, immune-enhancing dose of an mTOR inhibitor. The method includes acquiring a value of effectiveness to the CAR therapy, or sample suitability, wherein said value is indicative of the effectiveness or suitability of the CAR-expressing cell therapy.

In embodiments, the value of effectiveness to the CAR therapy, or sample suitability, comprises a measure of one, two, three, four, five, six or more (all) of the following:
(i) the level or activity of one, two, three, or more (e.g., all) of resting T_{EFF} cells, resting T_{REG} cells, younger T cells (e.g., younger CD4 or CD8 cells, or gamma/delta T cells), or early memory T cells, or a combination thereof, in a sample (e.g., an apheresis sample or a manufactured CAR-expressing cell product sample);
(ii) the level or activity of one, two, three, or more (e.g., all) of activated T_{EFF} cells, activated T_{REG} cells, older T cells (e.g., older CD4 or CD8 cells), or late memory T cells, or a combination thereof, in a sample (e.g., an apheresis sample or a manufactured CAR-expressing cell product sample);
(iii) the level or activity of an immune cell exhaustion marker, e.g., one, two or more immune checkpoint inhibitors (e.g., PD-1, PD-L1, TIM-3 and/or LAG-3) in a sample (e.g., an apheresis sample or a manufactured CAR-expressing cell product sample). In one embodiment, an immune cell has an exhausted phenotype, e.g., co-expresses at least two exhaustion markers, e.g., co-expresses PD-1 and TIM-3. In other embodiments, an immune cell has an exhausted phenotype, e.g., co-expresses at least two exhaustion markers, e.g., co-expresses PD-1 and LAG-3;
(iv) the level or activity of CD27 and/or CD45RO− (e.g., CD27+CD45RO−) immune effector cells, e.g., in a CD4+ or a CD8+ T cell population, in a sample (e.g., an apheresis sample or a manufactured CAR-expressing cell product sample);
(v) the level or activity of one, two, three, four, five, ten, twelve or more of the biomarkers chosen from CCL20, IL-17a and/or IL-6, PD-1, PD-L1, LAG-3, TIM-3, CD57, CD27, CD122, CD62L, KLRG1;
(vi) a cytokine level or activity (e.g., quality of cytokine repertoire) in a CAR-expressing cell product sample; or
(vii) a transduction efficiency of a CAR-expressing cell in a manufactured CAR-expressing cell product sample.

In some embodiments of any of the methods disclosed herein, the CAR-expressing cell therapy comprises a plurality (e.g., a population) of CAR-expressing immune effector cells, e.g., a plurality (e.g., a population) of T cells or NK cells, or a combination thereof. In one embodiment, the CAR-expressing cell therapy includes administration of a low, immune-enhancing dose of an mTOR inhibitor.

In some embodiments of any of the methods disclosed herein, the measure of one or more of (i)-(vii) is obtained from an apheresis sample acquired from the subject. The apheresis sample can be evaluated prior to infusion or re-infusion.

In some embodiments of any of the methods disclosed herein, the measure of one or more of (i)-(vii) is obtained from a manufactured CAR-expressing cell product sample. The manufactured CAR-expressing cell product can be evaluated prior to infusion or re-infusion.

In some embodiments of any of the methods disclosed herein, the subject is evaluated prior to receiving, during, or after receiving, the CAR-expressing cell therapy.

In some embodiments of any of the methods disclosed herein, the measure of one or more of (i)-(vii) evaluates a profile for one or more of gene expression, flow cytometry or protein expression.

In some embodiments of any of the methods disclosed herein, the method further comprises identifying the subject as a responder, a non-responder, a relapser or a non-relapser, based on a measure of one or more of (i)-(vii).

In some embodiments of any of the methods disclosed herein, a responder (e.g., a complete responder) has, or is identified as having, a greater level or activity of one, two, or more (all) of GZMK, PPF1BP2, or naïve T cells as compared to a non-responder.

In some embodiments of any of the methods disclosed herein, a non-responder has, or is identified as having, a greater level or activity of one, two, three, four, five, six, seven, or more (e.g., all) of IL22, IL-2RA, IL-21, IRF8, IL8, CCL17, CCL22, effector T cells, or regulatory T cells, as compared to a responder.

In an embodiment, a relapser is a patient having, or who is identified as having, an increased level of expression of one or more of (e.g., 2, 3, 4, or all of) the following genes, compared to non relapsers: MIR199A1, MIR1203, uc021ovp, ITM2C, and HLA-DQB1 and/or a decreased levels of expression of one or more of (e.g., 2, 3, 4, 5, 6, 7, 8, 9, 10, 11, or all of) the following genes, compared to non relapsers: PPIAL4D, TTTY10, TXLNG2P, MIR4650-1, KDM5D, USP9Y, PRKY, RPS4Y2, RPS4Y1, NCRNA00185, SULT1E1, and EIF1AY.

In some embodiments of any of the methods disclosed herein, a complete responder has, or is identified as having, a greater, e.g., a statistically significant greater, percentage of CD8+ T cells compared to a reference value, e.g., a non-responder percentage of CD8+ T cells.

In some embodiments of any of the methods disclosed herein, a complete responder has, or is identified as having, a greater percentage of CD27+CD45RO− immune effector cells, e.g., in the CD8+ population, compared to a reference value, e.g., a non-responder number of CD27+CD45RO− immune effector cells.

In some embodiments of any of the methods disclosed herein, a complete responder or a partial responder has, or is identified as having, a greater, e.g., a statistically significant greater, percentage of CD4+ T cells compared to a reference value, e.g., a non-responder percentage of CD4+ T cells.

In some embodiments of any of the methods disclosed herein, a complete responder has, or is identified as having, a greater percentage of one, two, three, or more (e.g., all) of resting T_{EFF} cells, resting T_{REG} cells, younger T cells (e.g., younger CD4 or CD8 cells, or gamma/delta T cells), or early memory T cells, or a combination thereof, compared to a reference value, e.g., a non-responder number of resting T_{EFF} cells, resting T_{REG} cells, younger T cells (e.g., younger CD4 or CD8 cells), or early memory T cells.

In some embodiments of any of the methods disclosed herein, a non-responder has, or is identified as having, a greater percentage of one, two, three, or more (e.g., all) of activated T_{EFF} cells, activated T_{REG} cells, older T cells (e.g., older CD4 or CD8 cells), or late memory T cells, or a combination thereof, compared to a reference value, e.g., a responder number of activated T_{EFF} cells, activated T_{REG} cells, older T cells (e.g., older CD4 or CD8 cells), or late memory T cells.

In some embodiments of any of the methods disclosed herein, a non-responder has, or is identified as having, a greater percentage of an immune cell exhaustion marker, e.g., one, two or more immune checkpoint inhibitors (e.g., PD-1, PD-L1, TIM-3 and/or LAG-3). In one embodiment, a non-responder has, or is identified as having, a greater percentage of PD-1, PD-L1, or LAG-3 expressing immune effector cells (e.g., CD4+ T cells and/or CD8+ T cells) (e.g., CAR-expressing CD4+ cells and/or CD8+ T cells) compared to the percentage of PD-1 or LAG-3 expressing immune effector cells from a responder.

In one embodiment, a non-responder has, or is identified as having, a greater percentage of immune cells having an exhausted phenotype, e.g., immune cells that co-express at least two exhaustion markers, e.g., co-expresses PD-1, PD-L1 and/or TIM-3. In other embodiments, a non-responder has, or is identified as having, a greater percentage of immune cells having an exhausted phenotype, e.g., immune cells that co-express at least two exhaustion markers, e.g., co-expresses PD-1 and LAG-3.

In some embodiments of any of the methods disclosed herein, a non-responder has, or is identified as having, a greater percentage of PD-1/PD-L1+/LAG-3+ cells in the CAR-expressing cell population compared to a responder (e.g., a complete responder) to the CAR-expressing cell therapy.

In some embodiments of any of the methods disclosed herein, a partial responder has, or is identified as having, a higher percentages of PD-1/PD-L1+/LAG-3+ cells, than a responder, in the CAR-expressing cell population.

In some embodiments of any of the methods disclosed herein, a non-responder has, or is identified as having, an exhausted phenotype of PD1/PD-L1+ CAR+ and co-expression of LAG3 in the CAR-expressing cell population.

In some embodiments of any of the methods disclosed herein, a non-responder has, or is identified as having, a greater percentage of PD-1/PD-L1+/TIM-3+ cells in the CAR-expressing cell population compared to the responder (e.g., a complete responder).

In some embodiments of any of the methods disclosed herein, a partial responders has, or is identified as having, a higher percentage of PD-1/PD-L1+/TIM-3+ cells, than responders, in the CAR-expressing cell population.

In some embodiments of any of the methods disclosed herein, the presence of CD8+CD27+CD45RO− T cells in an apheresis sample is a positive predictor of the subject response to a CAR-expressing cell therapy.

In some embodiments of any of the methods disclosed herein, a high percentage of PD1+ CAR+ and LAG3+ or TIM3+ T cells in an apheresis sample is a poor prognostic predictor of the subject response to a CAR-expressing cell therapy.

In some embodiments of any of the methods disclosed herein, the responder (e.g., the complete or partial responder) has one, two, three or more (or all) of the following profile:
(i) has a greater number of CD27+ immune effector cells compared to a reference value, e.g., a non-responder number of CD27+ immune effector cells;
(ii) has a greater number of CD8+ T cells compared to a reference value, e.g., a non-responder number of CD8+ T cells;
(iii) has a lower number of immune cells expressing one or more checkpoint inhibitors, e.g., a checkpoint inhibitor chosen from PD-1, PD-L1, LAG-3, TIM-3, or KLRG-1, or a combination, compared to a reference value, e.g., a non-responder number of cells expressing one or more checkpoint inhibitors; or
(iv) has a greater number of one, two, three, four or more (all) of resting T_{EFF} cells, resting T_{REG} cells, naïve CD4 cells, unstimulated memory cells or early memory T cells, or a combination thereof, compared to a reference value, e.g., a non-responder number of resting T_{EFF} cells, resting T_{REG} cells, naïve CD4 cells, unstimulated memory cells or early memory T cells.

In some embodiments of any of the methods disclosed herein, the cytokine level or activity of (vi) is chosen from one, two, three, four, five, six, seven, eight, or more (or all) of cytokine CCL20/MIP3a, IL17A, IL6, GM-CSF, IFNγ, IL10, IL13, IL2, IL21, IL4, IL5, IL9 or TNFα, or a combination thereof. The cytokine can be chosen from one, two, three, four or more (all) of IL-17a, CCL20, IL2, IL6, or TNFα. In one embodiment, an increased level or activity of a cytokine is chosen from one or both of IL-17a and CCL20, is indicative of increased responsiveness or decreased relapse.

In some embodiments of any of the methods disclosed herein, a transduction efficiency of 15% or higher in (vii) is indicative of increased responsiveness or decreased relapse.

In some embodiments of any of the methods disclosed herein, a transduction efficiency of less than 15% in (vii) is indicative of decreased responsiveness or increased relapse.

In embodiments, the responder, a non-responder, a relapser or a non-relapser identified by the methods herein can be further evaluated according to clinical criteria. For example, a complete responder has, or is identified as, a subject having a disease, e.g., a cancer, who exhibits a complete response, e.g., a complete remission, to a treatment. A complete response may be identified, e.g., using the NCCN Guidelines® (which are incorporated by reference herein in their entireties), as described herein. A partial responder has, or is identified as, a subject having a disease, e.g., a cancer, who exhibits a partial response, e.g., a partial remission, to a treatment. A partial response may be identified, e.g., using the NCCN Guidelines®, as described herein. A non-responder has, or is identified as, a subject having a disease, e.g., a cancer, who does not exhibit a response to a treatment, e.g., the patient has stable disease or progressive disease. A non-responder may be identified, e.g., using the NCCN Guidelines®, as described herein.

Alternatively, or in combination with the methods disclosed herein, responsive to said value, performing one, two, three, four or more of:
administering e.g., to a responder or a non-relapser, a CAR-expressing cell therapy;
administered an altered dosing of a CAR-expressing cell therapy;
altering the schedule or time course of a CAR-expressing cell therapy;
administering, e.g., to a non-responder or a partial responder, an additional agent in combination with a CAR-expressing cell therapy, e.g., a checkpoint inhibitor, e.g., a checkpoint inhibitor described herein;
administering to a non-responder or partial responder a therapy that increases the number of younger T cells in the subject prior to treatment with a CAR-expressing cell therapy;
modifying a manufacturing process of a CAR-expressing cell therapy, e.g., enriching for younger T cells prior to introducing a nucleic acid encoding a CAR, or increasing the transduction efficiency, e.g., for a subject identified as a non-responder or a partial responder;
administering an alternative therapy, e.g., for a non-responder or partial responder or relapser; or
if the subject is, or is identified as, a non-responder or a relapser, decreasing the T_{REG} cell population and/or T_{REG} gene signature, e.g., by one or more of CD25 depletion, administration of cyclophosphamide, anti-GITR antibody, or a combination thereof.

In certain embodiments, the subject is pre-treated with an anti-GITR antibody. In certain embodiment, the subject is treated with an anti-GITR antibody prior to infusion or re-infusion.

In some embodiments of the methods described herein, imaging with FDG-PET/CT (PET/CT) is performed on a subject who has been treated with a CAR therapy. This measurement can predict response to the therapy. For instance, in embodiments, metabolically active tumor volume (MTV) and/or [11F]-2-fluoro-2-deoxy-D-glucose (FDG) uptake are measured. In embodiments, a decrease in MTV is indicative of response, e.g., CR (complete response) or PR (partial response), e.g., a post-treatment MTV value of about 0 is indicative of CR, while an increase in MTV is indicative of PD (progressive disease). In embodiments, a decrease in FDG uptake is indicative of response, e.g., CR or PR, while an increase in FDG uptake is indicative of PD. In embodiments, the imaging is performed after administration of the CAR therapy, e.g., about 1 week, 2 weeks, 3 weeks, 4 weeks, 1 month, 2 months, 3 months, 4 months, 5 months, or 6 months after administration of the CAR therapy. In embodiments, the imaging is performed on a subject who does not have symptoms of CRS (cytokine release syndrome), e.g., a patient who suffered from CRS and whose symptoms resolved prior to imaging. In embodiments, the imaging is performed on a subject who has symptoms of CRS. In embodiments, imaging is performed prior to CAR therapy, and the pre-therapy image is compared to a post-therapy image. In embodiments, the subject has a cancer, e.g., lymphoma, e.g., diffuse large B-cell lymphoma (DLBCL) or follicular lymphoma (FL). In some embodiments, the CAR therapy comprises a CAR19-expressing cell, e.g., murine CTL019 or humanized CTL119 as described herein, e.g., Tables 2-3. In some embodiments, the CAR therapy comprises a CAR therapy described herein, e.g., a CAR20-expressing cell, a CAR22-expressing cell, or a CAR19-expressing cell, optionally in combination with a B-cell therapy.

### Personalized Medicine (Theranostics)

CD19 Characteristics, e.g. Mutations

Without wishing to be bound by theory, some cancer patients show an initial response to a CD19 inhibitor such as a CD19 CAR-expressing cell, and then relapse. In some embodiments, the relapse is caused (at least in part) by a frameshift and/or premature stop codon in CD19 in the cancer cells, or other change in the expression (including expression levels) of CD19 which reduces the ability of a CD19 CAR-expressing cell to target the cancer cells. Such a mutation can reduce the effectiveness of the CD19 therapy and contribute to the patient's relapse.

This application discloses, among other things, methods for treating a subject having cancer comprising one or more of: (1) determining if a subject has a difference, e.g., statistically significant difference, in a characteristic of CD19 relative to a reference characteristic, and (2) if there is a difference between the determined characteristic and reference characteristic, administering to the subject a therapeutically effective dose of a CAR therapy, e.g., CART, thereby treating the subject. The patient may be, e.g., a patient who has relapsed after treatment with a CD19 inhibitor, e.g., a CD19 CAR expressing cell. The patient may be a patient who has received or is receiving a CD19 CAR therapy and is at risk of relapse. The patient may be a non-responder to a CD19 CAR therapy.

In embodiments, the subject has or is identified as having a difference, e.g., a statistically significant difference, between a determined level compared to a reference level of one or more markers listed in Table 29 (on page 530 of International Application WO 2016/164731, filed Apr. 8, 2016, which is incorporated by reference in its entirety) in a biological sample.

In embodiments, the subject has or is identified as having a difference between a determined characteristic compared to a reference characteristic, in a characteristic of CD19, e.g., a mutation causing a frameshift or a premature stop codon or both, in a biological sample.

In embodiments, the subject has or is identified as having a difference, e.g., a statistically significant difference, between a determined level compared to a reference level of Treg cells in a biological sample.

Additional characteristics that can be measured to determine a therapeutically effectice dose of CAR therapy are described in pages 8-13, and 64-65 of International Application WO 2016/164731, filed Apr. 8, 2016, which is incorporated by reference in its entirety.

The characteristic can be, e.g., a CD19 sequence, e.g., protein or nucleic acid sequence. The sequence can be determined, e.g., as described in the Examples, by high throughput nucleic acid sequencing, or by mass spectrometry of proteins. As described in the Example herein, a patient may relapse after CD19 CART therapy because of mutations in CD19, e.g., in exon 2 of CD19, e.g., a mutation that causes a frameshift and a premature stop codon in CD19. In embodiments, the insertion or deletion does not cause one or both of a frameshift and a premature stop codon. The mutation may be, e.g., an insertion, a deletion, a substitution, a translocation, or a combination of any of the foregoing. The insertion, deletion, or substitution may involve, e.g., at least 1, 2, 3, 4, 5, 10, 15, 20, 20, or 50 nucleotides. The insertion, deletion, or substitution may involve, e.g., at most 2, 3, 4, 5, 10, 15, 20, 20, 50, or 100 nucleotides. In some cases, a population of cells will comprise more than one mutation. In such cases, the mutations can be in overlapping or non-overlapping sub-populations of cells.

In some cases a patient is identified as having a CD19 characteristic that reduces CD19's ability to engage with a CD19 inhibitor such as a CD19 CAR expressing cell. Such a characteristic may be, e.g., a frameshift mutation, a premature stop codon, an alteration in nucleic acid sequence or an alteration in the structure of the primary mRNA transcript. The characteristic may be, e.g., a departure from normal production of CD19 that occurs earlier than splicing. The characteristic may be, e.g., a characteristic other than exon skipping. Such patients may be treated with an inhibitor of another target, e.g., a B-cell inhibitor, for example a CAR expressing cell directed against another epitope, e.g., an epitope within one or more of CD10, CD20, CD22, CD34, CD123, FLT-3, or ROR1.

In some cases, a patient is identified as having a CD19 characteristic that reduces CD19's ability to engage with a CD19 inhibitor, such as a CD19 CAR expressing cell, but does not reduce or abrogate CD19's ability to engage with a second CD19 inhibitor, such as a CD19 inhibitor that binds to a different region on CD19. Such a characteristic may be, e.g., a mutation that does not cause one or both of a frameshift mutation or a premature stop codon. Such a characteristic may be, e.g., an alteration in nucleic acid sequence or an alteration in the structure of the primary mRNA transcript, a departure from normal production of CD19 that occurs earlier than splicing, or a characteristic other than exon skipping. Such patients may be treated with an inhibitor of CD19, e.g., a B-cell inhibitor directed against an intact region of CD19, e.g., a wild-type portion of CD19. For instance, if a mutation is present in exon 2, the second CD19 inhibitor may bind to an exon other than exon 2, or a part of exon 2 that lacks the mutation. The second CD19 inhibitor may be, e.g., a CD19 inhibitor described herein.

T_{EFF} and T_{REG} Signatures

Methods herein can include steps of determining a T_{REG} signature or determining the levels of T_{EFF} cells or T_{REG} cells, e.g., in a patient or in a population of cells e.g., immune cells. Methods herein can also include steps of reducing the level of T_{REG} cells, or decreasing a T_{REG} signature, in a patient or in a population of cells. In some embodiments, a T_{EFF} is a cell with upregulated expression of one or more (e.g., at least 10, 20, 30, 40, 50, 60, 70, 80, or all) of the following genes: AIM2, ALAS1, B4GALT5, BATF, C3orf26, C4orf43, CCL3, CCL4, CCT3, CCT7, CD40LG, CHAC2, CSF2, CTNNA1, EBNA1BP2, EDARADD, EEF1E1, EIF2B3, EIF2S1, FABP5, FAM40B, FKBP4, FOSL1, GFOD1, GLRX2, HSPD1, HSPE1, IFNG, IL15RA, IL21, IL2RA, IL3, KCNK5, KIAA0020, LARP4, LRP8, LTA, MANF, MIR1182, MIR155, MIR155HG, MTCH2, MYOF, NDUFAF1, NLN, NME1, NME1-NME2, OTUD7B, PAM, PDIA6, PEA15, PFKM, PGAM1, PGAM4, PPIL1, PRDX4, PRSS23, PSMD1, PSMD11, PSMD14, PTRH2, PUS7, RBBP8, RPF2, RPP25, SFXN1, SLC27A2, SLC39A14, SLC43A3, SORD, SPR, SRXN1, STIP1, STT3A, TBX21, TMCC2, TMEM165, TNFRSF9, TXN, TXNDCS, UCK2, VDR, WDR12, YWHAG, and ZDHHC16. In some embodiments, a T_{REG} cell is a cell with upregulated expression of one or more (e.g., at least 10, 20, 30, 40, 50, 60, 70, or all) of the following genes: AIM2, ALAS1, BATF, C5orf32, CCL17, CD40LG, CHAC2, CSF1, CTSL1, EBNA1BP2, EDARADD, EMP1, EPAS1, FABP5, FAM40B, FKBP4, FOSL1, GCLM, GK, GPR56, HMOX1, HSPD1, HSPE1, IKBIP, IL10, IL13, IL15RA, IL1RN, IL2RA, IL3, IL4, IL5, IL9, KCNK5, LTA, MANF, MIR1182, MIR155, MIR155HG, MYOF, NDUFAF1, NLN, NME1, NME1-NME2, PANX2, PDIA6, PGAM4, PPIL1, PPPDE2, PRDX4, PRKAR1B, PSMD1, PSMD11, PUS7, RBBP8, SLC27A2, SLC39A14, SLC43A3, SRXN1, STIP1, STT3A, TBX21, TNFRSF11A, TNFRSF1B, TNFRSF8, TNFRSF9, TXN, UCK2, VDR, VTRNA1-3, WDR12, YWHAG, ZDHHC16, and ZNF282. The upregulated expression may be, e.g., measured 16 hours after stimulation. The upregulated expression may be determined, e.g., by measuring RNA levels for the indicated genes.

In embodiments, the method comprises decreasing the T_{REG} signature in the subject prior to apheresis. In embodiments, the method further comprises decreasing the T_{REG} signature in the subject, e.g., by administering cyclophosphamide, an anti-GITR antibody, or both to the subject. In embodiments, the method comprises pre-treating a subject with cyclophosphamide, an anti-GITR antibody, or both, prior to collection of cells for CAR-expressing cell product manufacturing. In embodiments, the method further comprises obtaining a sample from the subject, wherein the sample comprises a cellular fraction (e.g., which comprises blood), a tissue fraction, an apheresis sample, or a bone marrow sample.

### Pharmaceutical Compositions and Treatments

Pharmaceutical compositions of the present invention may comprise, in some aspects, a CAR-expressing cell, e.g., a plurality of CAR-expressing cells, as described herein, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions of the present invention are in one aspect formulated for intravenous administration.

Pharmaceutical compositions of the present invention may be administered in a manner appropriate to the disease to be treated (or prevented). The quantity and frequency of administration will be determined by such factors as the condition of the patient, and the type and severity of the patient's disease, although appropriate dosages may be determined by clinical trials.

In one embodiment, the pharmaceutical composition is substantially free of, e.g., there are no detectable levels of a contaminant, e.g., selected from the group consisting of endotoxin, mycoplasma, replication competent lentivirus (RCL), p24, VSV-G nucleic acid, HIV gag, residual anti-CD3/anti-CD28 coated beads, mouse antibodies, pooled human serum, bovine serum albumin, bovine serum, culture media components, vector packaging cell or plasmid components, a bacterium and a fungus. In one embodiment, the bacterium is at least one selected from the group consisting of Alcaligenes faecalis, Candida albicans, Escherichia coli, Haemophilus influenza, Neisseria meningitides, Pseudomonas aeruginosa, Staphylococcus aureus, Streptococcus pneumonia, and Streptococcus pyogenes group A.

When “an immunologically effective amount,” “an anti-tumor effective amount,” “a tumor-inhibiting effective amount,” or “therapeutic amount” is indicated, the precise amount of the compositions of the present invention to be administered can be determined by a physician with consideration of individual differences in age, weight, tumor size, extent of infection or metastasis, and condition of the patient (subject). In some embodiments, a pharmaceutical composition comprising the cells, e.g., T cells described herein may be administered at a dosage of 10⁴ to 10⁹ cells/kg body weight, in some instances 10⁵ to 10⁶ cells/kg body weight, including all integer values within those ranges. In some embodiments, the cells, e.g., T cells described herein may be administered at 3×10⁴, 1×10⁶, 3×10⁶, or 1×10⁷ cells/kg body weight. The cell compositions may also be administered multiple times at these dosages. The cells can be administered by using infusion techniques that are commonly known in immunotherapy (see, e.g., Rosenberg et al., New Eng. J. of Med. 319:1676, 1988).

In some embodiments, a dose of CAR cells (e.g., CD19 or BCMA CAR cells) comprises about 1×10⁵, 2×10⁵, 5×10⁵, 1×10⁶, 1.1×10⁶, 2×10⁶, 3.6×10⁶, 5×10⁶, 1×10⁷, 1.8×10⁷, 2×10⁷, 5×10⁷, 1×10⁸, 2×10⁸, or 5×10⁸ cells/kg. In some embodiments, a dose of CAR cells (e.g., CD19 or BCMA CAR cells) comprises at least about 1×10⁵, 2×10⁵, 5×10⁵, 1×10⁶, 1.1×10⁶, 2×10⁶, 3.6×10⁶, 5×10⁶, 1×10⁷, 1.8×10⁷, 2×10⁷, 5×10⁷, 1×10⁸, 2×10⁸, or 5×10⁸ cells/kg. In some embodiments, a dose of CAR cells (e.g., CD19 or BCMA CAR cells) comprises up to about 1×10⁵, 2×10⁵, 5×10⁵, 1×10⁶, 1.1×10⁶, 2×10⁶, 3.6×10⁶, 5×10⁶, 1×10⁷, 1.8×10⁷, 2×10⁷, 5×10⁷, 1×10⁸, 2×10⁸, or 5×10⁸ cells/kg. In some embodiments, a dose of CAR cells (e.g., CD19 or BCMA CAR cells) comprises about 1.1×10⁶-1.8×10⁷ cells/kg or about 8×10⁵-1.5×10⁶ cells/kg. In some embodiments, a dose of CAR cells (e.g., CD19 or BCMA CAR cells) comprises about 1×10⁷, 2×10⁷, 5×10⁷, 1×10⁸, 2×10⁸, 5×10⁸, 1×10⁹, 2×10⁹, or 5×10⁹ cells. In some embodiments, a dose of CAR cells (e.g., CD19 or BCMA CAR cells) comprises at least about 1×10⁷, 2×10⁷, 5×10⁷, 1×10⁸, 2×10⁸, 5×10⁸, 1×10⁹, 2×10⁹, or 5×10⁹ cells. In some embodiments, a dose of CAR cells (e.g., CD19 or BCMA CAR cells) comprises up to about 1×10⁷, 2×10⁷, 5×10⁷, 1×10⁸, 2×10⁸, 5×10⁸, 1×10⁹, 2×10⁹, or 5×10⁹ cells.

In certain aspects, it may be desired to administer activated cells, e.g., T cells or NK cells, to a subject and then subsequently redraw blood (or have an apheresis performed), activate the cells therefrom according to the present invention, and reinfuse the patient with these activated and expanded cells. This process can be carried out multiple times every few weeks. In certain aspects, cells, e.g., T cells or NK cells, can be activated from blood draws of from 10 cc to 400 cc. In certain aspects, cells, e.g., T cells or NK cells, are activated from blood draws of 20 cc, 30 cc, 40 cc, 50 cc, 60 cc, 70 cc, 80 cc, 90 cc, or 100 cc.

The administration of the subject compositions may be carried out in any convenient manner, including by aerosol inhalation, injection, ingestion, transfusion, implantation or transplantation. The compositions described herein may be administered to a patient trans arterially, subcutaneously, intradermally, intratumorally, intranodally, intramedullary, intramuscularly, by intravenous (i.v.) injection, or intraperitoneally. In one aspect, the cell compositions, e.g., T cell or NK cell compositions, of the present invention are administered to a patient by intradermal or subcutaneous injection. In one aspect, the cell compositions e.g., T cell or NK cell compositions, of the present invention are administered by i.v. injection. The compositions of cells e.g., T cell or NK cell compositions, may be injected directly into a tumor, lymph node, or site of infection.

In an aspect, subjects may undergo leukapheresis, wherein leukocytes are collected, enriched, or depleted ex vivo to select and/or isolate the cells of interest, e.g., T cells. These cell isolates, e.g., T cell or NK cell isolates, may be expanded by methods known in the art and treated such that one or more CAR constructs of the invention may be introduced, thereby creating a CAR-expressing cell, e.g., CAR T cell of the invention. Subjects in need thereof may subsequently undergo standard treatment with high dose chemotherapy followed by peripheral blood stem cell transplantation. In certain aspects, following or concurrent with the transplant, subjects receive an infusion of the expanded CAR-expressing cells of the present invention. In an additional aspect, expanded cells are administered before or following surgery.

The dosage of the above treatments to be administered to a patient will vary with the precise nature of the condition being treated and the recipient of the treatment. The scaling of dosages for human administration can be performed according to art-accepted practices. The dose for a therapeutic, e.g., an antibody, e.g., CAMPATH, for example, may be, e.g., in the range 1 to about 100 mg for an adult patient, e.g., administered daily for a period between 1 and 30 days. A suitable daily dose is 1 to 10 mg per day although in some instances larger doses of up to 40 mg per day may be used (described in U.S. Pat. No. 6,120,766).

In one embodiment, the CAR is introduced into cells, e.g., T cells or NK cells, e.g., using in vitro transcription, and the subject (e.g., human) receives an initial administration of CAR-expressing cells, e.g., CAR T cells of the invention, and one or more subsequent administrations of the CAR-expressing cells, e.g., CAR T cells of the invention, wherein the one or more subsequent administrations are administered less than 15 days, e.g., 14, 13, 12, 11, 10, 9, 8, 7, 6, 5, 4, 3, or 2 days after the previous administration. In one embodiment, more than one administration of the CAR-expressing cells, e.g., CAR T cells of the invention are administered to the subject (e.g., human) per week, e.g., 2, 3, or 4 administrations of the CAR-expressing cells, e.g., CAR T cells of the invention are administered per week. In one embodiment, the subject (e.g., human subject) receives more than one administration of the CAR-expressing cells, e.g., CAR T cells per week (e.g., 2, 3 or 4 administrations per week) (also referred to herein as a cycle), followed by a week of no CAR-expressing cells, e.g., CAR T cells administrations, and then one or more additional administration of the CAR-expressing cells, e.g., CAR T cells (e.g., more than one administration of the CAR-expressing cells, e.g., CAR T cells per week) is administered to the subject. In another embodiment, the subject (e.g., human subject) receives more than one cycle of CAR-expressing cells, e.g., CAR T cells, and the time between each cycle is less than 10, 9, 8, 7, 6, 5, 4, or 3 days. In one embodiment, the CAR-expressing cells, e.g., CAR T cells are administered every other day for 3 administrations per week. In one embodiment, the CAR-expressing cells, e.g., CAR T cells of the invention are administered for at least two, three, four, five, six, seven, eight or more weeks.

In some embodiments, subjects may be adult subjects (i.e., 18 years of age and older). In certain embodiments, subjects may be between 1 and 30 years of age. In some embodiments, the subjects are 16 years of age or older. In certain embodiments, the subjects are between 16 and 30 years of age. In some embodiments, the subjects are child subjects (i.e., between 1 and 18 years of age).

In one aspect, CAR-expressing cells, e.g., CARTs are generated using lentiviral viral vectors, such as lentivirus. CAR-expressing cells, e.g., CARTs generated that way will have stable CAR expression.

In one aspect, CAR-expressing cells, e.g., CARTs, are generated using a viral vector such as a gammaretroviral vector, e.g., a gammaretroviral vector described herein. CARTs generated using these vectors can have stable CAR expression.

In one aspect, CAR-expressing cells, e.g., CARTs transiently express CAR vectors for 4, 5, 6, 7, 8, 9, 10, 11, 12, 13, 14, 15 days after transduction. Transient expression of CARs can be effected by RNA CAR vector delivery. In one aspect, the CAR RNA is transduced into the cell, e.g., NK cell or T cell, by electroporation.

A potential issue that can arise in patients being treated using transiently expressing CAR T cells (particularly with murine scFv bearing CARTs) is anaphylaxis after multiple treatments.

Without being bound by this theory, it is believed that such an anaphylactic response might be caused by a patient developing humoral anti-CAR response, i.e., anti-CAR antibodies having an anti-IgE isotype. It is thought that a patient's antibody producing cells undergo a class switch from IgG isotype (that does not cause anaphylaxis) to IgE isotype when there is a ten to fourteen day break in exposure to antigen.

If a patient is at high risk of generating an anti-CAR antibody response during the course of transient CAR therapy (such as those generated by RNA transductions), CART infusion breaks should not last more than ten to fourteen days.

### CAR22

Design, function and sequences of CAR22 constructs, and exemplary CAR22 constructs, are described, e.g., in pages 363-422 of International Application WO 2016/164731, filed Apr. 8, 2016, which is incorporated by reference in its entirety.

### CAR20 Constructs

Design, function and sequences of CAR20 constructs, and exemplary CAR20 constructs, are described, e.g., in pages 422-454 of International Application WO 2016/164731, filed Apr. 8, 2016, which is incorporated by reference in its entirety.

### CAR123 Constructs

Design, function and sequences of CAR123 constructs, and exemplary CAR123 constructs, are described, e.g., in pages 454-501 of International Application WO 2016/164731, filed Apr. 8, 2016, which is incorporated by reference in its entirety.

### Bispecific CAR19/CAR22 Constructs and Function Thereof

The production and function of bispecific CAR19/CAR22 constructs is described, e.g., on pages 501-506 of International Application WO 2016/164731, filed Apr. 8, 2016, which is incorporated by reference in its entirety. The anti-CD19 base molecule is a humanized anti-CD19 sequence, provided as construct ID 104876 of Table 2, which uses the LH orientation.

The nucleotide and amino acid sequences of CAR19/CAR22 constructs, are provided in Table 28 on pages 501-508 of International Application WO 2016/164731.

### EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples specifically point out various aspects of the present invention, and are not to be construed as limiting in any way the remainder of the disclosure.

### Example 1: Tandem Induction Radiation and Chimeric Antigen Receptor T Cell Therapy in Patients with Relapsed or Refractory Non-Hodgkin Lymphoma

### Introduction

Chimeric antigen receptor-modified T cell (CAR-T) therapies targeting lymphoma-specific epitopes have demonstrated efficacy in relapsed/refractory non-Hodgkin lymphoma. Radiotherapy (RT) can be used as bridging therapy for symptomatic progression, and/or as lymphodepletion prior to CAR-T infusion. The tandem sequencing of RT and CAR-T as disclosed in this Example has not been previously disclosed.

### Methods

Patients enrolled in a phase IIA study evaluating autologous T cells engineered to express CD19-directed CAR (NCT02030834) were divided into no RT or RT prior to infusion groups, including: induction RT (RT administered <30 days prior to CAR-T), prior RT (RT administered >30 days but <12 months prior to CAR-T), and remote RT (RT administered >12 months prior to CAR-T). As described in this Example, CAR-T in vivo expansion, side effects, and outcomes were determined. Median follow-up was defined as time from CAR-T infusion to event or last follow-up (f/u). Descriptive statistics were used to summarize data with Kaplan-Meier analysis for progression-free (PFS) and overall survival (OS).

### Results

Forty-one patients were evaluable and had the following characteristics: 18 no RT, 5 induction RT, 7 prior RT, and 11 remote RT. Patients included those with diffuse large B cell lymphoma (61%), follicular lymphoma (34%), and mantle cell lymphoma (5%). Induction RT was used to manage symptoms and was incorporated into the lymphodepleting regimen in those patients. Patients in the induction RT group began RT after T cell collection, e.g., on average 12 days prior to re-infusion (range 7-19); those in the prior RT group received RT e.g., on average 146 days prior to T cell collection (range 99-263). Median follow-up time was 674 days for all patients. One-year PFS and OS for each group were: 44% and 65% for theno RT group, 78% and 100% for the induction RT group, 0% and 86% for the prior RT group, and 61% and 90% for the remote RT group, respectively. Cytokine release syndrome (CRS), e.g., of more than grade 3, occurred in 10 of 41 patients overall (24%), but in no patient in the induction RT group (0 of 5). CAR-T expansion and day of peak CAR-T were not affected by RT given at any interval prior to T cell collection or re-infusion.

### Conclusions

This Example shows that induction RT prior to CAR-T infusion, e.g., does not impact the efficacy of CAR-T therapy, and may be associated with, e.g., a lower incidence of CRS. Given that RT can both palliate symptoms and be used in lymphodepletion regimens, this tandem approach warrants further exploration.

### Example 2: Chimeric Antigen Receptor T-Cell Therapy in Pediatric/Adolescent/Young Adult Patients with Acute Lymphoblastic Leukemia in First Relapse after Allogeneic Stem Cell Transplant in CR1

Background:

Patients with B-cell acute lymphoblastic leukemia (ALL) who relapse after first allogeneic stem cell transplant (alloSCT) have a poor prognosis. A recent study using the CIBMTR database reported a median survival of 7.4 months (95% CI, 6.0-9.6 months) (Crotta A., et al., (2017) Current Medical Research and Opinion 34(3):435-440). ELIANA and ENSIGN trials of the chimeric antigen receptor (CAR) T-cell therapy tisagenlecleucel (CAR19) in pediatric and adolescent/young adult (AYA) patients with relapsed/refractory (R/R) ALL included patients who relapsed after alloSCT (54%). Most relapsed patients were in a 2nd relapse or more, e.g., 3^{rd} relapse, 4^{th} relapse or 5^{th} relapse. Here we evaluate the outcomes of patients who received tisagenlecleucel following their first relapse after alloSCT performed in first complete remission (CR1).

Objective:

This Example describes the results of a study designed to examine efficacy and safety outcomes in this subset of patients who received tisagenlecleucel for first relapse following alloSCT in CR1.

Methods:

Pooled data from 2 single-arm, multicenter, phase 2 trials (ELIANA, N=75 [NCT02435849] and ENSIGN, N=58 [NCT02228096]) evaluated the efficacy and safety of tisagenlecleucel in pediatric and AYA patients with R/R ALL. Five patients received alloSCT as part of their initial therapy in CR1. After relapsing after alloSCT, they received tisagenlecleucel and were thus considered to be in their first relapse.

Results:

All 5 patients who received tisagenlecleucel in first relapse underwent chemotherapy after initial diagnosis followed by alloSCT in CR1. One patient received a transplant from a matched unrelated donor while the other 4 received transplants from matched siblings. Patients relapsed 4.5, 5.8, 12.4, 17.5 and 18.6 months following alloSCT with myeloablative conditioning. Blast counts at enrollment were 7%, 29%, 34%, 73%, and 79%; in the pooled ELIANA/ENSIGN cohort (N=133), and bone marrow blasts ranged from 5% to 99%. Four patients were male—aged 4, 7, 9 and 10 years at enrollment. One patient was female—aged 18 years at enrollment. Infusion with tisagenlecleucel took place 1.3, 1.5, 2.0, 3.1, and 4.4 months after relapse, a range comparable to that in the ELIANA/ENSIGN cohort (1-14 months). Four of 5 patients achieved minimal residual disease (MRD)-negative CRs (overall remission rate, 80%). One patient had unknown overall response because cerebral spinal fluid assessment was not done, although MRD-negative bone marrow remission was observed. Durations of remission in patients who achieved complete response (CR) starting from onset of remission were 3.2+ months (proceeded to SCT while in remission), 7.5+ months (non-SCT new therapy while in remission), 14.2+ months (ongoing remission), and 29.6+ months (non-SCT new therapy for secondary malignancy). All patients were alive at the end of the analysis. Overall survival was 1.8+, 10.8+, 15.1+, 18.8+, and 33.3+ months after infusion. Only 1 patient experienced cytokine release syndrome (CRS) (grade 3), and no patients experienced neurological toxicity. In the pooled cohort, 79% of patients experienced CRS (41% grade 3 or 4), and 37% experienced neurological events of any grade. The data from all 5 patients is summarized in Table 6.

TABLE 6
Characteristics of Patients Treated with Tisagenlecleucel
in First Relapse Following AlloSCT in CR1.

| | | | | | Patient |
|---|---|---|---|---|---|
| | Patient 1 | Patient 2 | Patient 3^{a} | Patient 4 | 5 |
| Age, years | 7 | 10 | 9 | 4 | 18 |
| Transplant | MUD | MSD | MSD | MSD | MSD |
| Time from | 4.5 | 12.4 | 18.6 | 17.5 | 5.8 |
| transplant to | | | | | |
| relapse, months | | | | | |
| Time from | 4.4 | 1.5 | 2.0 | 1.3 | 3.1 |
| relapse to | | | | | |
| infusion, | | | | | |
| months | | | | | |
| Best overall | CR | CR | CR | CR | UKN^{b} |
| response | | | | | |
| MRD status | Negative | Negative | Negative | Negative | — |
| Overall | 10.8+ | 18.8+ | 15.1+ | 33.3+ | 1.8+ |
| survival, | | | | | |
| months | | | | | |
| Maximum | None | Grade 3 | None | None | None |
| grade CRS | | | | | |
| Neurotoxicity | None | None | None | None | None |

^{a}Patient had Philadelphia chromosome-positive ALL.
^{b}Bone marrow remission was observed but cerebral spinal fluid assessment was not made.
AE, adverse event;
CR, complete remission;
CRS, cytokine release syndrome;
MRD, minimal residual disease;
MSD, matched sibling donor;
MUD, matched unrelated donor;
UKN, unknown.

### Example 3: Analysis of Patients with Relapsed or Refractory Non-Hodgkin Lymphoma Who Received Tandem Induction Radiation and Chimeric Antigen Receptor T Cell Therapy

This Example describes the exploratory analysis of patients in the NCT02030834 trial who were treated with CAR19-expressing cell therapy. Patients were treated with lymphodepleting therapy, e.g., chemotherapy or radiation therapy, or a combination thereof, prior to administration of CAR19-expressing cell therapy.

Patients who had been administered radiation therapy (RT) as part of the lymphodepleting therapy were evaluated as described in Table 7. In some embodiments, patients had no RT, remote RT (e.g., more than 12 months prior to administration of CAR19-expressing cell therapy), prior RT (e.g., more than 30 day but less than 12 months prior to administration of CAR19-expressing cell therapy, e.g., recent radiotherapy, e.g., prior to apheresis), or tandem/induction RT (e.g., less than 30 days prior to administration of CAR19-expressing cell therapy, e.g., after apheresis). In some embodiments, tandem/induction RT is also used as a lymphodepleting chemotherapy.

TABLE 7
Patient demographics

| | |
|---|---|
| Age, median (range) | 56 (24-77) |
| Sex, n (%) | |
| Male | 25 (61) |
| Female | 16 (39) |
| Previoustherapies, median (range) | 5 (1-10) |
| Pathologic subtype, n (%) | |
| DLBCL | 25 (61) |
| Follicular | 14 (34) |
| Mantle | 2 (5) |
| Prior radiotherapy, n (%) | |
| Yes | 23 (56) |
| No | 18 (44)) |
| Timing of radiotherapy, n (%) | |
| NoRT | 18 (44) |
| Remote RT (>1 year) | 11 (27) |
| Prior RT (>30 days) | 7 (17) |
| Tandem/Bridging (<30 days) | 5 (12) |
| Indications for bridging therapy | |
| Symptomatic progression | 4 |
| Pain | 1 |
| Fractionation | |
| 40 Gy, 2Gy/fraction | 2 |
| 4 Gy, 2Gy/fraction | 2 |
| 22 Gy, 2,2 Gy/fraction | 1 |

### Results:

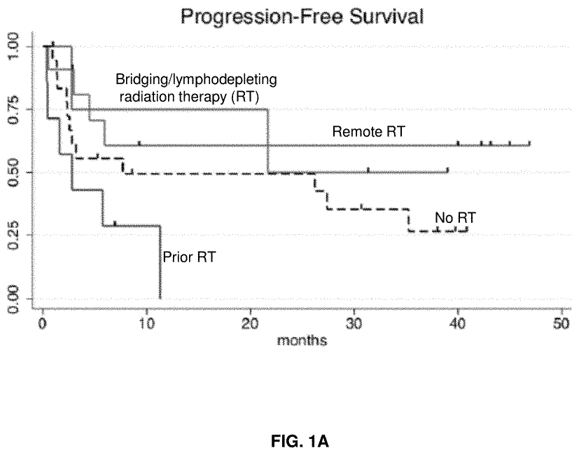

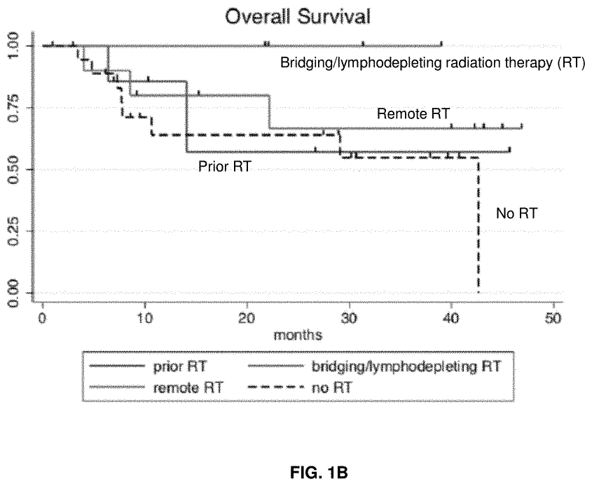

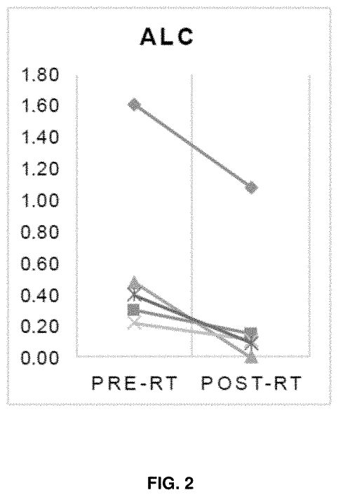

Patients who received tandem/induction RT prior to CAR19 therapy, had improved progression free survival (PFS) and overall survival (OS) compared to patients who received no RT, remote RT or prior RT (FIGS. 1A-1B and Table 8). In patients who had tandem/induction RT (e.g., bridging radiotherapy), the absolute lymphocyte count (ALC) was lower post-RT as compared to levels pre-RT (FIG. 2). The pre-RT ALC median was about 0.4, e.g., in the range of about 0.22 to 1.61. The post-RT ALC median was about 0.13, e.g., in the range of about 0.09-1.08.

With respect to cytokine release syndrome and neurotoxicity, patients who received tandem/induction RT, e.g., bridging radiotherapy, demonstrated, e.g., no or low CRS, e.g., grade 3 or higher CRS (Table 9). Patients who received tandem/induction RT, e.g., bridging radiotherapy, also demonstrated low levels of neurotoxicity.

TABLE 8
Survival analysis in patients receiving RT

| | PFS (days) | OS (days) |
|---|---|---|
| No RT | 196 (28-1239) | 578 (105-1296) |
| RemoteRT (>1 year) | 279 (15-1422) | 674 (87-1422) |
| Prior RT (>30 days) | 85 (11-344) | 315 (195-1386) |
| Tandem/Bridging | 659 (26-1183) | 669 (26-1183) |

TABLE 9
CRS in patients receiving RT

| Grade 3+ CRS | n | % |
|---|---|---|
| No RT | 5 | 28% |
| RemoteRT (>1 | 3 | 27% |
| year) | | |
| Prior RT (>30 days) | 2 | 29% |
| Tandem/bridging | 0 | 0% |

This example demonstrates, e.g., that radiation therapy (RT) as a bridging therapy in tandem with CAR-expressing cell therapy, e.g., CAR19-expressing cell therapy, is safe. In some embodiments, radiation therapy does not affect the expansion and/or proliferation of CAR-expressing cells. The exploratory analysis documented in this example illustrates that, e.g., patients treated with RT as a tandem/induction RT, e.g., bridging therapy, did not demonstrate CRS, e.g., severe CRS, e.g., grade 3 or higher CRS, after demonstration of a CAR-expressing cell therapy, e.g., CD19 CAR-expressing cell therapy.

### EQUIVALENTS

The disclosures of each and every patent, patent application, and publication cited herein are hereby incorporated herein by reference in their entirety. While this invention has been disclosed with reference to specific aspects, it is apparent that other aspects and variations of this invention may be devised by others skilled in the art without departing from the true spirit and scope of the invention. The appended claims are intended to be construed to include all such aspects and equivalent variations.

1. A method of treating a subject comprising administering to the subject a CAR-expressing cell therapy, e.g., a CAR19 expressing cell therapy, wherein the CAR-expressing cell therapy is administered less than 30 days, e.g., less than 29, 28, 27, 26, 25, 24, 23, 22, 21, 20, 19, 18, 17, 16, 15, 14, 13, 12, 11, 10, 9, 8, 7, 6, 5, 4, 3, 2, or 1 days, after administration of a lymphodepleting therapy comprising radiotherapy.

2. A method of treating, e.g., preventing, cytokine release syndrome (CRS) with a CAR-expressing cell therapy, e.g., a CAR19 expressing cell therapy, in a subject in need thereof, comprising administering to the subject a lymphodepleting therapy comprising radiotherapy, thereby preventing CRS in the subject.

3. The method of claim 2, wherein the radiotherapy is administered less than 30 days, e.g., less than 29, 28, 27, 26, 25, 24, 23, 22, 21, 20, 19, 18, 17, 16, 15, 14, 13, 12, 11, 10, 9, 8, 7, 6, 5, 4, 3, 2, or 1 days, prior to the administration of the CAR-expressing cell therapy.

4. The method of claim 2, wherein the subject (i) is at risk of developing, has, or is diagnosed with CRS; (ii) is identified or has previously been identified as being at risk for CRS; and/or (iii) has been, is being, or will be administered a CAR therapy, e.g., a CD19 CAR-expressing cell.

5. The method of claim 2, wherein the subject is selected based on
(i) risk of developing CRS, and/or
(ii) whether the subject has been, is being, or will be administered a CAR therapy (e.g., CD19 CAR-expressing cell).

6. The method of claim 2, wherein the subject is selected for administration of radiotherapy (i) if the subject is at risk of developing CRS or (ii) if the subject will be administered a CAR therapy, e.g., a CD19 CAR-expressing cell.

7. (canceled)

8. The method of claim 2, wherein the CRS is (i) a severe CRS, e.g., grade 4 or 5 CRS or (ii) a less than severe CRS, e.g., grade 1, 2, or 3 CRS.

9. (canceled)

10. A method of treating a subject comprising administering to the subject a CAR-expressing cell therapy, e.g., a CAR19 expressing cell therapy, wherein the CAR-expressing cell therapy is administered after stem cell therapy (SCT), e.g., autologous SCT or allogeneic SCT, wherein the subject has not responded, e.g., relapsed, to the SCT therapy, thereby treating the subject.

11. The method of claim 10, wherein the CAR-expressing cell therapy is administered after relapse from SCT, e.g., about 1-6 months (e.g., about 1.1-1.5, 1.5-2.0, 2.0-2.5, 2.5-3, 3-3.5, 3.5-4, 4-4.5. 4.5-5, 5-5.5, or 5.5-6 months) after relapse.

12. The method of claim 10, wherein the subject has a response, e.g., remission, a complete response, or a partial response, to the CAR-expressing cell therapy; optionally wherein the subject in remission has a minimal residual disease (MRD) negative remission, e.g., MRD negative bone marrow remission.

13. (canceled)

14. The method of claim 10, wherein:
(i) the SCT is allogeneic SCT; or
(ii) the SCT is administered as a first-line therapy or second-line therapy.

15. (canceled)

16. The method of claim 10, wherein the subject is administered SCT, e.g., alloSCT, in first complete remission (CR1); optionally wherein the subject is in (i) first relapse after SCT or (ii) a 2nd relapse or more, e.g., 3^{rd}, 4^{th} or 5^{th} relapse.

17. (canceled)

18. (canceled)

19. The method of claim 10, wherein the subject
(i) had been previously administered a chemotherapy, e.g., as described herein;
(ii) is a pediatric patient e.g., aged about 18 years of age or younger (e.g., 18, 17, 16, 15, 14, 13, 12, 11, 10, 9, 8, 7, 6, 5, 4, 3, 2, 1 or younger (e.g., 12 months, 6 months, 3 months or less));
(iii) is a young adult (e.g., aged about 18-35 years); or
(iv) is an adolescent, e.g., aged about 10-19 years, e.g., about 10, 11, 12, 13, 14, 15, 16, 17, 18 or 19 years);
optionally wherein the subject is a mammal, e.g., a human.

20-22. (canceled)

23. The method of claim 10, wherein no response to, or relapse from SCT is determined by evaluating the presence, e.g., reappearance, of cancer cells in the subject, e.g., in the blood or bone marrow; optionally wherein the presence, e.g., reappearance, of cancer cells comprises detection of the cancer cells at or above a threshold, e.g., above 20%, 1%, 10%, 5%, 4%, 3%, 2%, or 1%.

24. (canceled)

25. The method of claim 1, wherein:
(i) the CAR-expressing cell therapy, e.g., CAR19 expressing cell therapy, comprises a plurality of cells;
(ii) the CAR-expressing cell therapy, e.g., CAR19 expressing cell therapy, is administered in a single infusion or a split-dose infusion; or
(iii) the CAR19-expressing cell therapy is administered at a dosage of about 1×10⁸, 2×10⁸, 3×10⁸, 4×10⁸, 5×10⁸, 6×10⁸, 7×10⁸, 8×10⁸, or 9×10⁸ cells, e.g., about 5×10⁸ cells, e.g., about 5×10⁸ cells in a single infusion.

26-28. (canceled)

29. The method of claim 1, wherein the CAR19-expressing cell therapy comprises:
(a) a cell (e.g., a population of cells) expressing a murine CAR molecule that binds to CD19 comprising:
(i) one or more of (e.g., all three of) heavy chain complementary determining region 1 (HCDR1), HCDR2, and HCDR3 of any CD19 scFv domain amino acid sequence listed in Table 3 and one or more of (e.g., all three of) light chain complementary determining region 1 (LCDR1), LCDR2, and LCDR3 of any CD19 scFv domain amino acid sequence listed in Table 3;
(ii) a heavy chain variable region (VH) of any CD19 scFv domain amino acid sequence listed in Table 3 and a light chain variable region (VL) of any CD19 scFv domain amino acid sequence listed in Table 3;
(iii) a CD19 scFv domain amino acid sequence listed in Table 3 (e.g., SEQ ID NO: 59, 109, 111, or 114); or
(iv) a full-length CD19 CAR amino acid sequence listed in Table 3 (e.g., SEQ ID NO: 110, 112, 113, or 115, or residues 22-486 of SEQ ID NO: 58); or
(b) a cell expressing a humanized CAR molecule that binds to CD19 comprising:
(i) one or more of (e.g., all three of) heavy chain complementary determining region 1 (HCDR1), HCDR2, and HCDR3 of any CD19 scFv domain amino acid sequence listed in Table 2 and one or more of (e.g., all three of) light chain complementary determining region 1 (LCDR1), LCDR2, and LCDR3 of any CD19 scFv domain amino acid sequence listed in Table 2;
(ii) a heavy chain variable region (VH) of any CD19 scFv domain amino acid sequence listed in Table 2 and a light chain variable region (VL) of any CD19 scFv domain amino acid sequence listed in Table 2;
(iii) a CD19 scFv domain amino acid sequence listed in Table 2 (e.g., any one of SEQ ID NOs: 1-12); or
(iv) a full-length CD19 CAR amino acid sequence listed in Table 2 (e.g., residues 22-486 of any one of SEQ ID NOs: 31-34 or 42, or residues 22-491 of any one of SEQ ID NOs: 35-41).

30. (canceled)

31. The method of claim 29, wherein the CAR molecule comprises:
(i) a scFv;
(ii) a transmembrane domain that comprises a transmembrane domain of a protein selected from the group consisting of the alpha, beta or zeta chain of the T-cell receptor, CD28, CD3 epsilon, CD45, CD4, CD5, CD8, CD9, CD16, CD22, CD33, CD37, CD64, CD80, CD86, CD134, CD137 and CD154;
(iii) a hinge region comprising SEQ ID NO:14, or a sequence with 95-99% identity thereof;
(iv) a costimulatory domain that is a functional signaling domain obtained from a protein selected from the group consisting of OX40, CD2, CD27, CD28, CDS, ICAM-1, LFA-1 (CD11a/CD18), ICOS (CD278), and 4-1BB (CD137), wherein optionally the costimulatory domain comprises the amino acid sequence of SEQ ID NO:16 or 51;
(v) an intracellular signaling domain comprising a functional signaling domain of 4-1BB and/or a functional signaling domain of CD3 zeta; e.g., an intracellular signaling domain comprising the sequence of SEQ ID NO: 16 and/or the sequence of SEQ ID NO:17 or 43; or
(vi) a leader sequence, optionally wherein the leader sequence comprises the amino acid sequence of SEQ ID NO: 13.

32. The method of claim 1, wherein the cell comprising a CAR comprises a nucleic acid encoding the CAR; optionally wherein the nucleic acid encoding the CAR is a lentiviral vector or an RNA, e.g., an in vitro transcribed RNA; and/or optionally wherein the nucleic acid encoding the CAR is introduced into the cells by lentiviral transduction or by electroporation.

33-36. (canceled)

37. The method of claim 1, wherein the cell (e.g., population of cells) is a T cell or NK cell; optionally wherein the T cell is an autologous or allogeneic T cell.

38. (canceled)

39. (canceled)

40. The method of claim 1, wherein the subject has a cancer, e.g., a solid tumor or a hematological cancer, e.g., a lymphoma or a leukemia; optionally wherein the cancer is a hematological cancer chosen from acute leukemia, B-cell acute lymphoid leukemia (B-ALL), T-cell acute lymphoid leukemia (T-ALL), small lymphocytic leukemia (SLL), acute lymphoid leukemia (ALL), chronic leukemia, chronic myelogenous leukemia (CML), chronic lymphocytic leukemia (CLL), non-Hodgkin lymphoma (NHL), e.g., relapsed/refractory NHL, or multiple myeloma.

41-43. (canceled)
